SEQUENCE LISTING (6 sequences)

SEQ ID NO: 1 - DNA, 16 bp, Synthetic construct
  source 1..16: mol_type = other DNA
  modified_base 1: mod_base = OTHER; note = 2'-O,4'-C-methylene guanosine 3'-phosphorothioate
  modified_base 2: mod_base = OTHER; note = 2'-O,4'-C-methylene 5-methylcytidine 3'-phosphorothioate
  modified_base 3: mod_base = OTHER; note = 2'-O,4'-C-methylene adenosine 3'-phosphorothioate
  modified_base 4: mod_base = OTHER; note = 2'-deoxythymidine 3'-phosphorothioate
  modified_base 5: mod_base = OTHER; note = 2'-deoxythymidine 3'-phosphorothioate
  modified_base 6: mod_base = OTHER; note = 2'-deoxy 5-methylcytidine 3'-phosphorothioate
  modified_base 7: mod_base = OTHER; note = 2'-deoxythymidine 3'-phosphorothioate
  modified_base 10: mod_base = OTHER; note = 2'-deoxythymidine 3'-phosphorothioate
  modified_base 8: mod_base = OTHER; note = 2'-deoxyadenosine 3'-phosphorothioate
  modified_base 9: mod_base = OTHER; note = 2'-deoxyadenosine 3'-phosphorothioate
  modified_base 11: mod_base = OTHER; note = 2'-deoxyadenosine 3'-phosphorothioate
  modified_base 12: mod_base = OTHER; note = 2'-deoxyguanosine 3'-phosphorothioate
  modified_base 13: mod_base = OTHER; note = 2'-deoxy 5-methylcytidine 3'-phosphorothioate
  modified_base 14: mod_base = OTHER; note = 2'-O,4'-C-methylene adenosine 3'-phosphorothioate
  modified_base 15: mod_base = OTHER; note = 2'-O,4'-C-methylene guanosine 3'-phosphorothioate
  modified_base 16: mod_base = OTHER; note = 2'-O,4'-C-methylene 5-methylcytidine
gcattctaatagcagc

SEQ ID NO: 2 - RNA, 23 nt, Synthetic construct
  source 1..23: mol_type = other RNA
  modified_base 2: mod_base = OTHER; note = 2'-O-methylguanosine 5'-phosphorothioate
  modified_base 3: mod_base = cm
  modified_base 5: mod_base = cm
  modified_base 8: mod_base = cm
  modified_base 18: mod_base = cm
  modified_base 4: mod_base = um
  modified_base 13: mod_base = um
  modified_base 14: mod_base = um
  modified_base 17: mod_base = um
  modified_base 21: mod_base = um
  modified_base 6: mod_base = OTHER; note = 2'-O-methyladenosine
  modified_base 7: mod_base = OTHER; note = 2'-O-methyladenosine
  modified_base 9: mod_base = OTHER; note = 2'-O-methyladenosine
  modified_base 16: mod_base = OTHER; note = 2'-O-methyladenosine
  modified_base 19: mod_base = OTHER; note = 2'-O-methyladenosine
  modified_base 22: mod_base = OTHER; note = 2'-O-methyladenosine 3'-phosphorothioate
  modified_base 15: mod_base = gm
  modified_base 20: mod_base = gm
  modified_base 11: mod_base = OTHER; note = 2'-fluoroadenosine
  modified_base 10: mod_base = OTHER; note = 2'-fluorouridine
  modified_base 12: mod_base = OTHER; note = 2'-fluorouridine
  misc_feature 1: note = The 3' end of the inverted abasic site is bonded to a phosphate group which is itself bonded to a hexylamine end cap
  modified_base 1: mod_base = OTHER; note = Inverted abasic site
  modified_base 23: mod_base = OTHER; note = Inverted abasic site
ngctcaacatatttgatcagtan

SEQ ID NO: 3 - RNA, 21 nt, Synthetic construct
  source 1..21: mol_type = other RNA
  modified_base 1: mod_base = OTHER; note = 2'-O-methyluridine 3'-phosphorothioate
  modified_base 4: mod_base = OTHER; note = 2'-fluorouridine
  modified_base 12: mod_base = OTHER; note = 2'-fluorouridine
  modified_base 14: mod_base = OTHER; note = 2'-fluorouridine
  modified_base 16: mod_base = OTHER; note = 2'-fluorouridine
  modified_base 17: mod_base = um
  modified_base 2: mod_base = OTHER; note = 2'-fluoroadenosine 3'-phosphorothioate
  modified_base 6: mod_base = OTHER; note = 2'-fluoroadenosine
  modified_base 10: mod_base = OTHER; note = 2'-fluoroadenosine
  modified_base 9: mod_base = OTHER; note = 2'-O-methyladenosine
  modified_base 11: mod_base = OTHER; note = 2'-O-methyladenosine
  modified_base 13: mod_base = OTHER; note = 2'-O-methyladenosine
  modified_base 19: mod_base = OTHER; note = 2'-O-methyladenosine
  modified_base 18: mod_base = OTHER; note = 2'-fluoroguanosine
  modified_base 20: mod_base = OTHER; note = 2'-fluoroguanosine 3'-phosphorothioate
  modified_base 8: mod_base = OTHER; note = 2'-fluorocytidine
  modified_base 3: mod_base = OTHER; note = 2'-O-methylcytidine 3'-phosphorothioate
  modified_base 5: mod_base = gm
  modified_base 15: mod_base = gm
  modified_base 21: mod_base = cm
  modified_base 7: mod_base = um
tactgatcaaatatgttgagc

SEQ ID NO: 4 - RNA, 18 nt, Synthetic construct
  source 1..18: mol_type = other RNA
  modified_base 1: mod_base = OTHER; note = 2'-O-methylcytidine 3'-phosphorothioate
  modified_base 11: mod_base = OTHER; note = 2'-O-methylcytidine
  modified_base 2: mod_base = OTHER; note = 2'-fluoroadenosine 3'-phosphorothioate
  modified_base 6: mod_base = OTHER; note = 2'-fluoroadenosine
  modified_base 12: mod_base = OTHER; note = 2'-fluoroadenosine
  modified_base 18: mod_base = OTHER; note = 2'-fluoroadenosine
  modified_base 3: mod_base = gm
  modified_base 15: mod_base = gm
  modified_base 17: mod_base = OTHER; note = 2'-O-methylguanosine 3'-phosphorothioate
  modified_base 4: mod_base = OTHER; note = 2'-fluorocytidine
  modified_base 10: mod_base = OTHER; note = 2'-fluorocytidine
  modified_base 14: mod_base = OTHER; note = 2'-fluorocytidine
  modified_base 5: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 16: mod_base = OTHER; note = 2'-fluorouridine 3'-phosphorothioate
  modified_base 9: mod_base = um
  modified_base 13: mod_base = um
cagcaaattccatcgtga

SEQ ID NO: 5 - RNA, 20 nt, Synthetic construct
  source 1..20: mol_type = other RNA
  modified_base 6: mod_base = OTHER; note = 2'-fluorocytidine
  modified_base 12: mod_base = OTHER; note = 2'-fluorocytidine
  modified_base 16: mod_base = OTHER; note = 2'-fluorocytidine
  modified_base 2: mod_base = OTHER; note = 2'-fluoroadenosine 3'-phosphorothioate
  modified_base 4: mod_base = OTHER; note = 2'-fluoroadenosine
  modified_base 8: mod_base = OTHER; note = 2'-fluoroadenosine
  modified_base 14: mod_base = OTHER; note = 2'-fluoroadenosine
  modified_base 20: mod_base = OTHER; note = 2'-fluoroadenosine
  modified_base 10: mod_base = OTHER; note = 2'-fluorouridine
  modified_base 18: mod_base = OTHER; note = 2'-fluorouridine 3'-phosphorothioate
  modified_base 3: mod_base = OTHER; note = 2'-O-methylcytidine
  modified_base 13: mod_base = OTHER; note = 2'-O-methylcytidine
  modified_base 5: mod_base = gm
  modified_base 17: mod_base = gm
  modified_base 19: mod_base = OTHER; note = 2'-O-methylguanosine 3'-phosphorothioate
  modified_base 11: mod_base = um
  modified_base 15: mod_base = um
  modified_base 1: mod_base = OTHER; note = 2'-O-methyladenosine 3'-phosphorothioate
  modified_base 7: mod_base = OTHER; note = 2'-O-methyladenosine
  modified_base 9: mod_base = OTHER; note = 2'-O-methyladenosine
aacagcaaattccatcgtga

SEQ ID NO: 6 - RNA, 20 nt, Synthetic construct
  source 1..20: mol_type = other RNA
  modified_base 1: mod_base = OTHER; note = 5'-phosphate 2'-O-methyluridine 3'-phosphorothioate
  modified_base 19: mod_base = OTHER; note = 2'-O-methyluridine 3'-phosphorothioate
  modified_base 17: mod_base = um
  modified_base 13: mod_base = um
  modified_base 7: mod_base = um
  modified_base 2: mod_base = OTHER; note = 2'-fluorocytidine 3'-phosphorothioate
  modified_base 4: mod_base = OTHER; note = 2'-fluorocytidine
  modified_base 16: mod_base = OTHER; note = 2'-fluorocytidine
  modified_base 6: mod_base = OTHER; note = 2'-fluoroadenosine
  modified_base 10: mod_base = OTHER; note = 2'-fluoroadenosine
  modified_base 3: mod_base = OTHER; note = 2'-O-methyladenosine
  modified_base 11: mod_base = OTHER; note = 2'-O-methyladenosine
  modified_base 5: mod_base = gm
  modified_base 9: mod_base = gm
  modified_base 15: mod_base = gm
  modified_base 8: mod_base = OTHER; note = 2'-fluoroguanosine
  modified_base 18: mod_base = OTHER; note = 2'-fluoroguanosine 3'-phosphorothioate
  modified_base 12: mod_base = OTHER; note = 2'-fluorouridine
  modified_base 14: mod_base = OTHER; note = 2'-fluorouridine
  modified_base 20: mod_base = OTHER; note = 2'-fluorouridine
tcacgatggaatttgctgtt

(19) United States
(12) Patent Application Publication
(10) Pub. No.: US 2025/0064944 A1
(43) Pub. Date: Feb. 27, 2025

(54) MULTIVALENT CARGO-CARRYING COMPLEXES AND USES THEREOF

(71) Applicant: ASTRAZENECA AB, Södertälje (SE)

(72) Inventors: Prasad WAKCHAURE, Gothenburg (SE); Lars Anders DAHLÉN, Gothenburg (SE); Daniel Laurent KNERR, Gothenburg (SE); Josephus Johannes WETERINGS, Gothenburg (SE); Shalini ANDERSSON, Gothenburg (SE)

(21) Appl. No.: 18/780,813

(22) Filed: Jul. 23, 2024

(30) Foreign Application Priority Data
Jul. 24, 2023 (GB) 2311334.3

(51) Int. Cl. A61K 47/54; C12N 15/113

(52) U.S. Cl. CPC A61K 47/549; A61K 47/548; C12N 15/113; C12N 2310/11; C12N 2310/14; C12N 2310/3511

(57) ABSTRACT

Provided are multivalent saccharide-containing compounds which are bonded to nucleic acids. These compounds contain a shikimic acid-derived core and are useful for delivering nucleic acids to cells or tissues, e.g. for use in therapeutic treatments. Also provided are pharmaceutical compositions comprising the aforementioned compounds and medical uses of the same, including their use in treating or preventing conditions such as liver diseases.

Multivalent saccharide-containing compounds are provided which are bonded to nucleic acids. These compounds are useful for delivering nucleic acids to cells or tissues, e.g. for use in therapeutic treatments. In particular, the compounds comprise specific monosaccharides which are capable of binding to asialoglycoprotein receptors (ASGPRs). Such compounds may be used, for example, in the targeted delivery of therapeutic oligonucleotides to cells such as liver cells. Also provided are pharmaceutical compositions comprising the aforementioned compounds and medical uses of the same, including their use in treating or preventing conditions such as liver diseases.

### CROSS REFERENCE TO RELATED APPLICATIONS

This specification claims the benefit of priority to GB Patent Application No. 2311334.3 (filed 24 Jul. 2023). The entire text of the above-referenced patent application is incorporated by reference into this specification.

### SEQUENCE LISTING

The present application is being filed along with a Sequence Listing in electronic format. The Sequence Listing is provided as a file entitled P75225WO ASG-104-WO-PCT Sequence listing .xml, created on Jul. 19, 2024, which is 66,807 bytes in size. The information in the electronic format of the sequence listing is incorporated by reference in its entirety.

### SUMMARY

There are numerous ways to target the delivery of cargo moieties, including therapeutic nucleic acids, to cells. One approach is to tether the cargo to one or more saccharide ligands to take advantage of saccharide-binding proteins associated with the target cells. Exemplary saccharide-binding proteins include cell surface receptors such as glycoprotein receptors, including asialoglycoprotein receptors (ASGPRs). ASGPRs have been shown to be highly expressed on the surface of mammalian hepatocytes, as well as carcinoma cell lines; they are also expressed on other cell types, albeit generally at a lower level. As such, ASGPRs represent a promising target for hepatic delivery of cargo, including therapeutic nucleic acid agents (see, e.g., D'Souza et al., J. Control Release, (2015) 203:126-139). Ligands which can be used for targeting ASGPRs include monosaccharides such as N-acetyl galactosamine (GalNAc).

Target-binding complexes often contain more than one ligand and/or more than one cargo moiety. This functionality can be achieved by the use of branched core structures with linkers being used to couple the ligands and/or the cargo moieties to the core. In this way, a complex can be prepared which has the appropriate number and arrangement of functional parts for the desired use. Examples of multivalent, e.g. branched, complexes are described in International Patent Publications: WO 2014/179620 (Isis Pharmaceuticals, Inc.); WO 2015/177668 A1 (Pfizer Inc.); WO 2009/073809 A2 (Alnylam Pharmaceuticals, Inc.); WO 2012/083046 A2 (Arrowhead Research Corporation); WO 2017/156012 A1 (Arrowhead Pharmaceuticals, Inc.); WO 2016/100401 A1 (Dicerna Pharmaceuticals, Inc.); WO 2017/174657 A1 (Silence Therapeutics GmbH); and WO 2019/092280 A1 (Silence Therapeutics GmbH). Complexes of the aforementioned types make use of different coupling chemistries to link the ligands and cargo moieties to the branched core structure, and also make use of different core structures. There is, however, a need for alternative chemical functionalities to couple cargo moieties and ligands in order to prepare complexes useful in delivering cargo moieties to cells. The present disclosure seeks to address this need by providing novel compounds and complexes, e.g. for use in targeting therapeutic nucleic acids to cells such as liver cells.

In brief, the compounds and complexes of the present disclosure contain one or more monosaccharide ligands and one or more nucleic acids. These are joined together using a multidentate ‘splitter’ which is derived from, or chemically related to, shikimic acid. The use of shikimic acid, and analogues thereof, in preparing the branching unit provides a novel way of coupling targeting and cargo moieties which may have advantages over known methods, including for example the use of simple, natural precursors. The compounds of the invention exhibit useful properties such as, e.g., improved targeting, increased activity and/or improved pharmacokinetic properties in vivo.

### SUMMARY OF THE DISCLOSURE

The present disclosure includes the following aspects and embodiments which are presented as numbered clauses 1 to 50:
1. A compound having the structure of Formula (I):

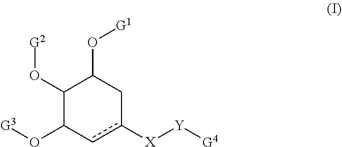

or a pharmaceutically acceptable salt thereof, wherein:
X is selected from a covalent bond, —CH₂—, and —C(O)—;
Y is —O— or —NR—,
wherein R is selected from —H and —(C₁-C₆)alkyl;
[symbol] represents a carbon-carbon single bond or double bond, with the proviso that when X is a covalent bond [symbol] represents a carbon-carbon single bond; and
G¹ to G⁴ each independently represents a -[spacer]-[ligand] moiety or a -[tether]-[linker]-[cargo] moiety, wherein each ligand is independently a monosaccharide and each cargo is independently a nucleic acid, with the proviso that at least one of G¹ to G⁴ represents a -[spacer]-[ligand] moiety and at least one of G¹ to G⁴ represents a -[tether]-[linker]-[cargo] moiety.

2. The compound or pharmaceutically acceptable salt thereof of clause 1, wherein X is —C(O)—.

3. The compound or pharmaceutically acceptable salt thereof of clause 1 or clause 2, wherein Y is —NR—, e.g., wherein Y is —NH— or —N(CH₃)—.

4. The compound or pharmaceutically acceptable salt thereof of any one of clauses 1-3, wherein [symbol] represents a single bond.

5. The compound or pharmaceutically acceptable salt thereof of any one of clauses 1-4, wherein each ligand is N-acetyl galactosamine (GalNAc).

6. The compound or pharmaceutically acceptable salt thereof of any one of clauses 1-5, wherein each cargo is independently selected from an antisense oligonucleotide (ASO), an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide and a G-quadruplex.

7. The compound or pharmaceutically acceptable salt thereof of any one of clauses 1-6, wherein each spacer independently comprises a chain of 2-20 atoms selected from C, N, O, S and P, e.g., wherein each spacer is independently selected from a linear alkylene (which may optionally be interrupted by one or more amide and/or phosphate groups) and a polyethylene glycol.

8. The compound or pharmaceutically acceptable salt thereof of any one of clauses 1-7, wherein [linker-tether]together in each case independently represents a moiety comprising a linear chain of 8 to 30 atoms.

9. The compound or pharmaceutically acceptable salt thereof of any one of clauses 1-8, wherein the compound has the structure of Formula (Ia) or Formula (Ib):

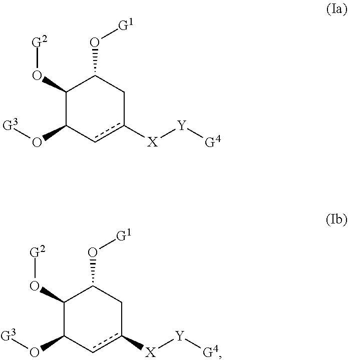

wherein X, Y, G¹, G², G³ and G⁴ are as defined in any of the preceding clauses, wherein [symbol] in Formula (Ib) represents a carbon-carbon single bond.

10. The compound or pharmaceutically acceptable salt thereof of any one of clauses 1-3 and 5-8, wherein the compound has the structure of Formula (Id):

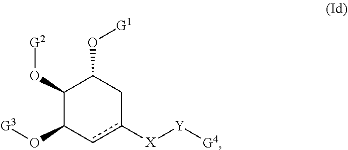

wherein X, Y, G¹, G², G³ and G⁴ are as defined in any of the preceding clauses, and [symbol] represents a double bond.

11. A compound having the structure of Formula (II):

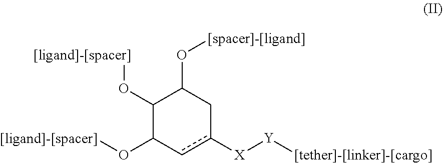

or a pharmaceutically acceptable salt thereof, wherein:
X is selected from a covalent bond, —CH₂—, and —C(O)—;
Y is —O— or —NR—,
   wherein R is selected from —H and —(C₁-C₆)alkyl;
[symbol] represents a carbon-carbon single bond or double bond, with the proviso that when X is a covalent bond [symbol] represents a carbon-carbon single bond;
spacer in each case independently represents a moiety comprising a linear chain of 6 to 20 atoms (e.g., selected from C, N, O, S and P) which attaches the ligand to the rest of the molecule;
ligand in each case is independently a monosaccharide;
linker and tether taken together represent a moiety which attaches the cargo to the rest of the molecule, e.g. comprising a linear chain of 8 to 30 atoms; and
cargo is a nucleic acid.

12. The compound or pharmaceutically acceptable salt thereof of clause 11, wherein X is —C(O)— and Y is —NR— (e.g., wherein R is —H or —CH₃).

13. The comp d or pharmaceutically acceptable salt thereof of clause 11 or clause 12, wherein [symbol] represents a single bond.

14. The compound or pharmaceutically acceptable salt thereof of any one of clauses 11-13, wherein the compound has the structure of Formula (IIa) or Formula (IIb):

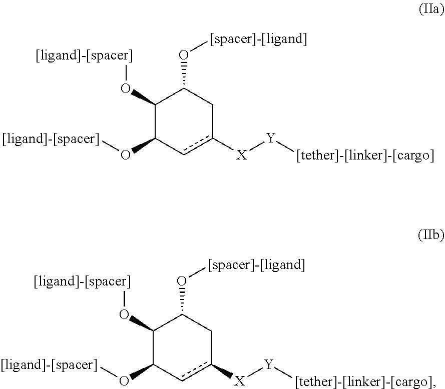

wherein X, Y, ligand, spacer, tether, linker and cargo are as defined in any of the preceding clauses, wherein [symbol] in Formula (IIb) represents a carbon-carbon single bond.

15. The compound or pharmaceutically acceptable salt thereof of clause 11 or clause 12, wherein the compound has the structure of Formula (IId):

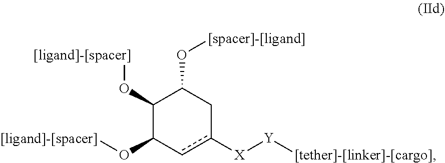

wherein X, Y, ligand, spacer, tether, linker and cargo are as defined in any of the preceding clauses, and [symbol] represents a double bond.

16. A compound having the structure of Formula (III):

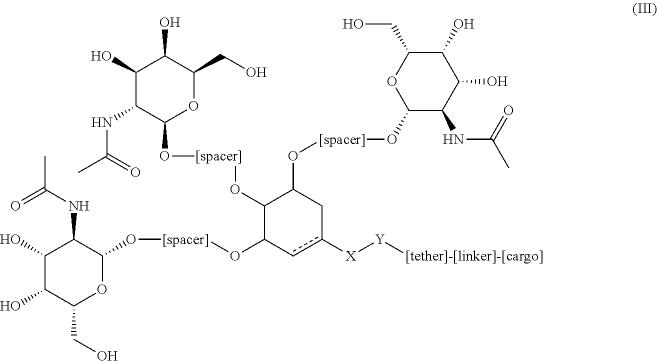

or a pharmaceutically acceptable salt thereof, wherein X, Y, spacer, tether, linker and cargo are as defined in any of the preceding clauses, and [symbol] represents a carbon-carbon single bond or double bond, with the proviso that when X is a covalent bond [symbol] represents a carbon-carbon single bond.

17. The compound or pharmaceutically acceptable salt thereof of clause 16, wherein:
X is —C(O)—;
Y is —O— or —NR—, wherein R is selected from —H and —(C₁₋₆)alkyl;
[symbol] represents a single bond;
spacer in each case independently represents a moiety comprising a linear chain of 6 to 20 atoms selected from C, N, O, S and P;
linker and tether taken together represent a moiety comprising a linear chain of 8 to 30 atoms; and
cargo is a nucleic acid.

18. The compound or pharmaceutically acceptable salt thereof of clause 16 or clause 17, wherein the compound has the structure of Formula (IIIa) or Formula (IIIb):

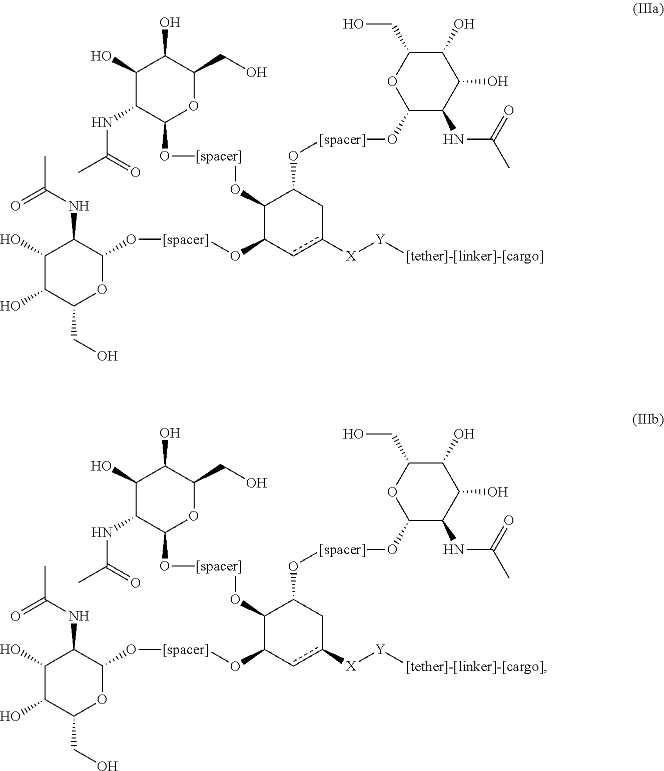

wherein X, Y, spacer, tether, linker and cargo are as defined in any of the preceding clauses, wherein [symbol] in Formula (IIIb) represents a carbon-carbon single bond.

[symbol]

19. The compound or pharmaceutically acceptable salt thereof of clause 16, wherein the compound has the structure of Formula (IIId):

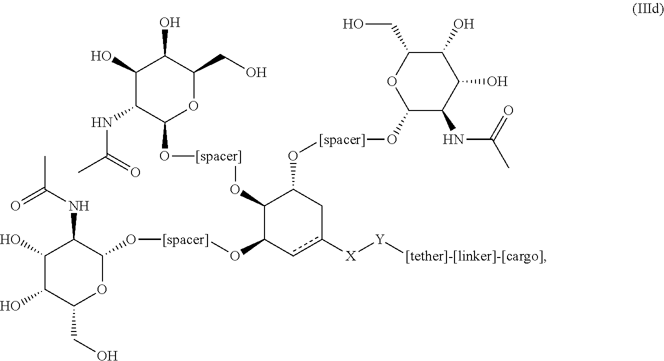

wherein X, Y, spacer, tether, linker and cargo are as defined in any of the preceding clauses, and [symbol] represents a double bond.

20. A compound having the structure of Formula (IV):

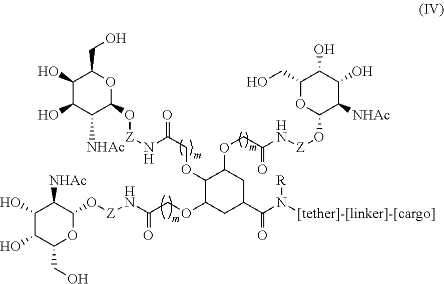

or a pharmaceutically acceptable salt thereof, wherein:
Z in each case independently represents a moiety comprising a linear chain of 1 to 16 atoms (e.g., selected from C, N, O, S and P);
m in each case is independently selected from the integers from 1 to 6;
R is selected from —H and —(C₁-C₆)alkyl; and
tether, linker and cargo are as defined in any of the preceding clauses.

21. The compound or pharmaceutically acceptable salt thereof of clause 20, wherein Z in each case is independently selected from *—(C₁-C₁₆)alkylene-, *—(C₂-C₁₆)alkenylene-, *—(C₁-C₁₂)alkylene-C(O)—, *—(C₂-C₁₂)alkenylene-C(O)—, *—(C₁-C₅)alkylene-C(O)NR′—(C₁-C₆)alkylene-, *—(C₂-C₈)alkenylene-C(O)NR′—(C₁-C₆)alkylene-, *—(C₁-C₈)alkylene-C(O)NR′—(C₂-C₆)alkenylene- and *—(C₂-C₈)alkenylene-C(O)NR′—(C₂-C₆)alkenylene- (where * in each case denotes the point of attachment to the GalNac oxygen atom), wherein R′ in each case is independently selected from —H and —(C₁-C₆)alkyl.

22. The compound or pharmaceutically acceptable salt thereof of clause 20 or clause 21, wherein each Z is independently selected from *—(C₁-C₆)alkylene-C(O)NR′—(C₁-C₄)alkylene- (wherein R′ in each case is independently selected from —H and —(C₁-C₆)alkyl), e.g. wherein Z is*—(C₄)alkylene-C(O)NH—(C₃)alkylene-.

23. The compound or pharmaceutically acceptable salt thereof of any one of clauses 20-22, wherein m in each case is independently selected from 1, 2, 3 and 4.

24. The compound or pharmaceutically acceptable salt thereof of any one of clauses 20-23, wherein the compound has the structure of Formula (IVa) or Formula (IVb):

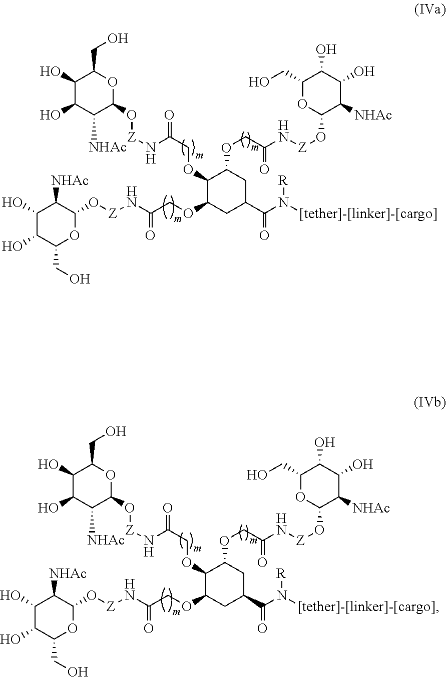

wherein Z, m, R, tether, linker and cargo are as defined in any of the preceding clauses.

25. A compound having the structure of Formula (V):

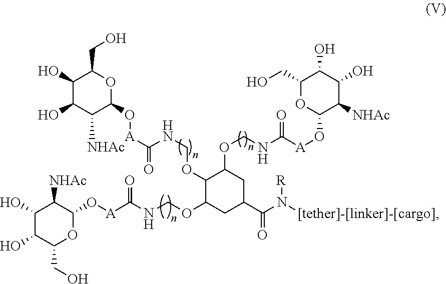

or a pharmaceutically acceptable salt thereof, wherein:
A in each case independently represents a moiety comprising a linear chain of 1 to 16 atoms (e.g., selected from C, N, O, S and P);
n in each case is independently selected from the integers from 1 to 6;
R is selected from —H and —(C₁-C₆)alkyl; and
tether, linker and cargo are as defined in any of the preceding clauses.

26. The compound or pharmaceutically acceptable salt thereof of clause 25, wherein A in each case is independently selected from *—(C₁-C₁₆)alkylene-, and *—(C₂-C₁₆)alkenylene- (where * in each case denotes the point of attachment to the GalNac oxygen atom).

27. The compound or pharmaceutically acceptable salt thereof of clause 25 or clause 26, wherein n in each case is independently selected from 1, 2, 3 and 4.

28. The compound or pharmaceutically acceptable salt thereof of any one of clauses 25-27, wherein the compound has the structure of Formula (Va) or Formula (Vb):

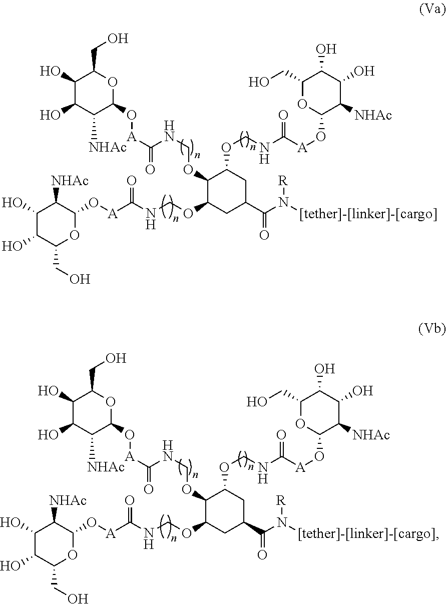

wherein A, n, R, tether, linker and cargo are as defined in any of the preceding clauses.

29. A compound having the structure of Formula (IX):

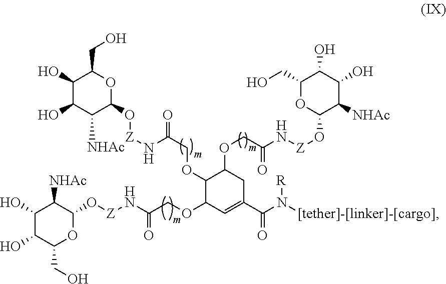

or a pharmaceutically acceptable salt thereof, wherein:
Z in each case independently represents a moiety comprising a linear chain of 1 to 16 atoms (e.g., selected from C, N, O, S and P) which attaches the GalNAc ligand to the rest of the molecule;
m in each case is independently selected from the integers from 1 to 6;
R is selected from —H and —(C₁-C₆)alkyl; and
tether, linker and cargo are as defined in any of the preceding clauses.

30. A compound having the structure of Formula (XI):

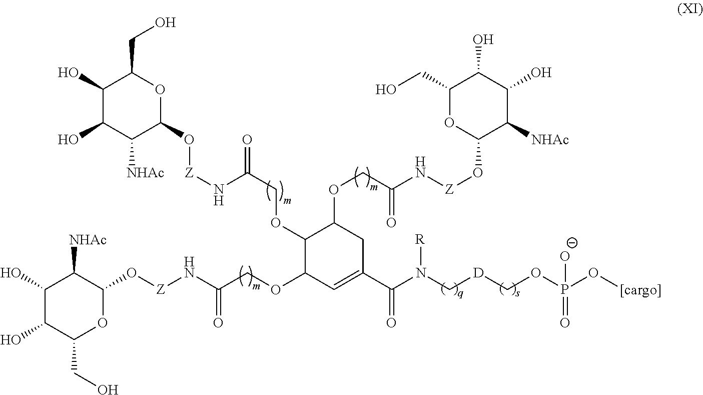

wherein:
Z in each case is independently selected from *—(C₁-C₁₂)alkylene- and *—(C₁-C₆)alkylene-C(O)NR′—(C₁-C₄)alkylene-, wherein R′ in each case is independently selected from —H and —(C₁-C₆)alkyl (where * in each case denotes the point of attachment to the GalNac oxygen atom);
m in each case is independently selected from the integers from 1 to 6;
R is selected from —H and —(C₁-C₆)alkyl;
q is an integer from 1 to 12;
D is selected from (i) —C(O)NH—, and (ii) —NHC(O)—(CH₂)—C(O)NH— (wherein t is an integer from 1 to 6);
s is an integer from 0 to 12; and cargo is as defined herein (e.g., an ASO or siRNA).

31. A compound selected from:

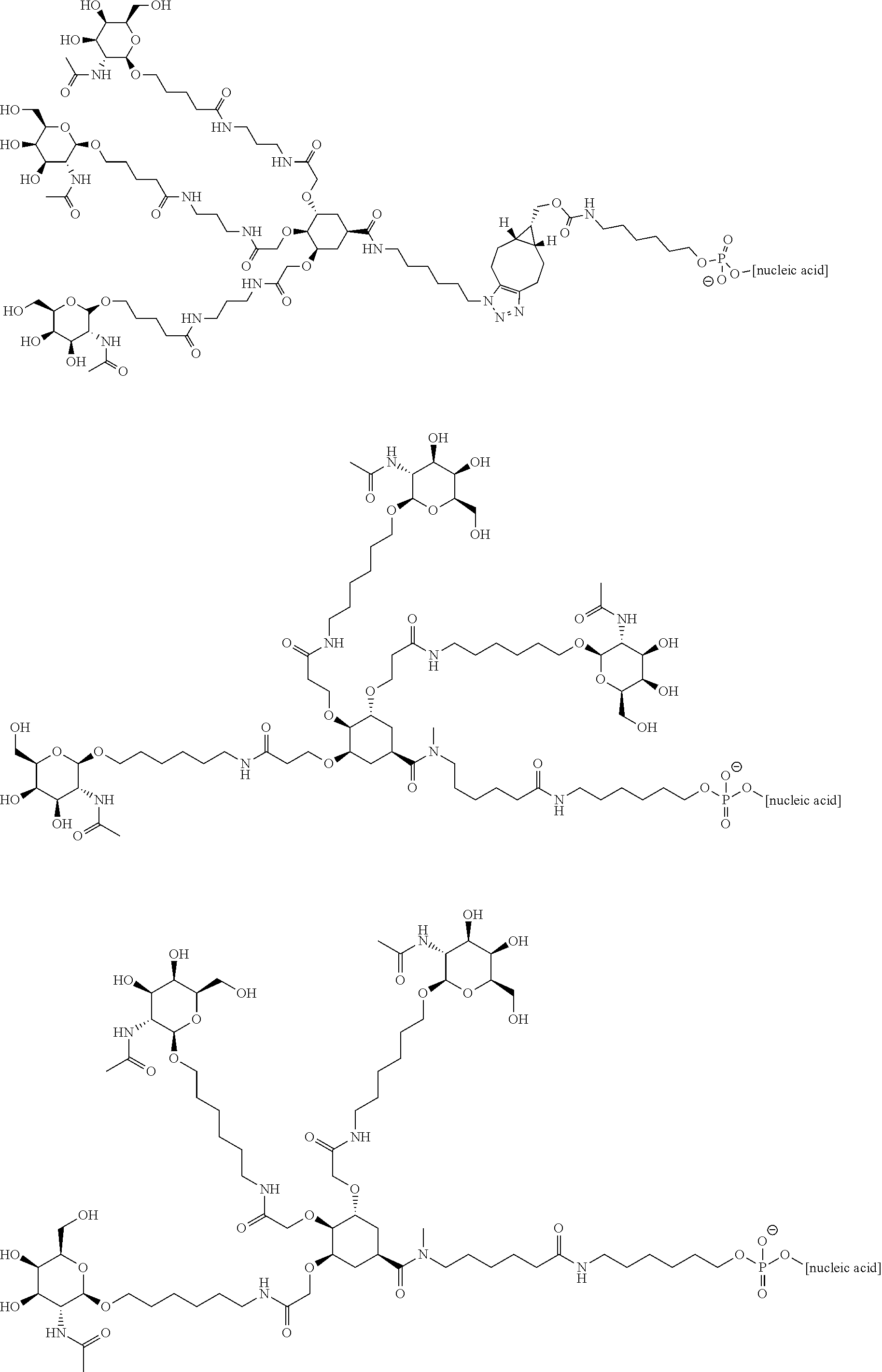

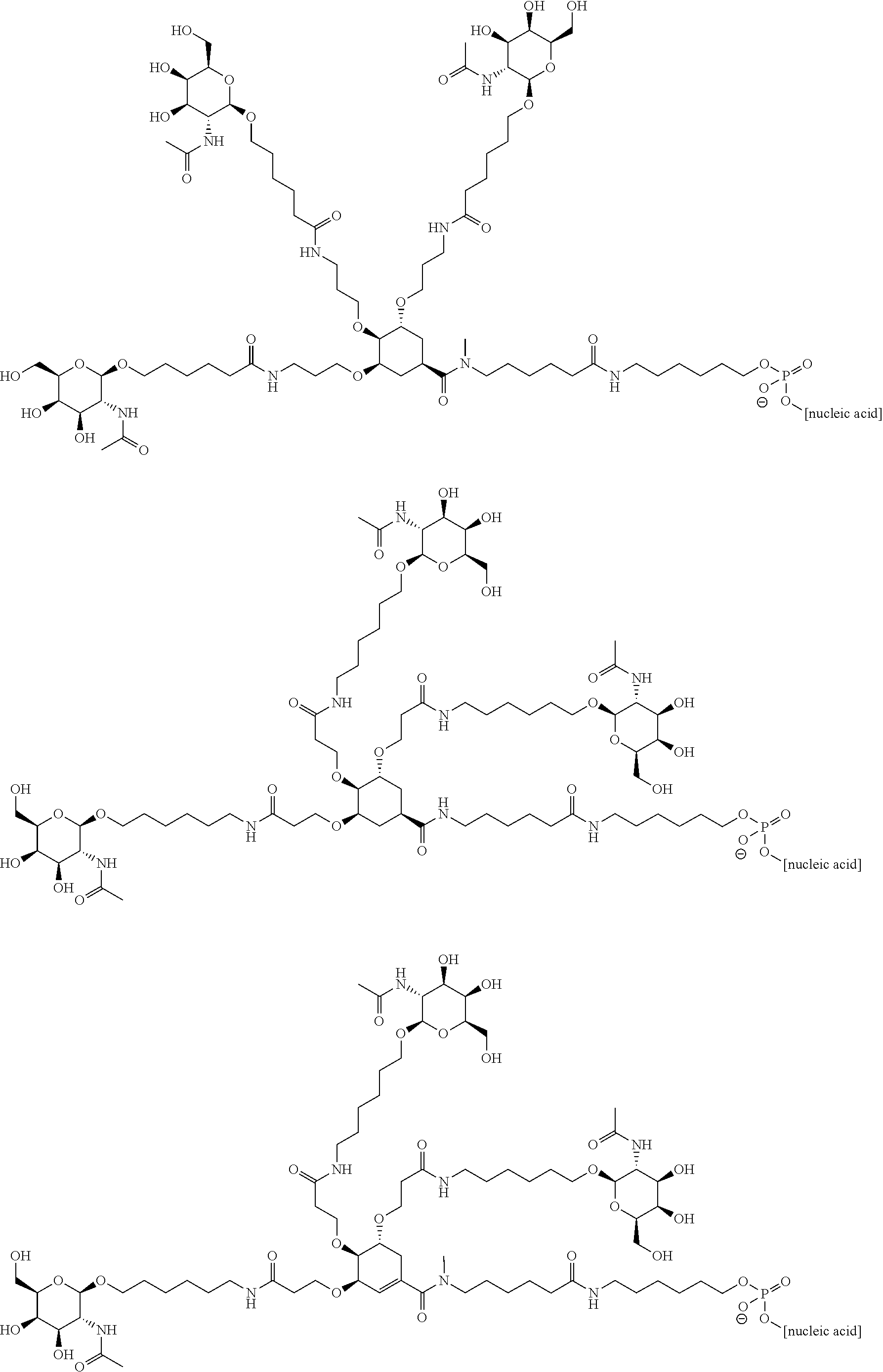

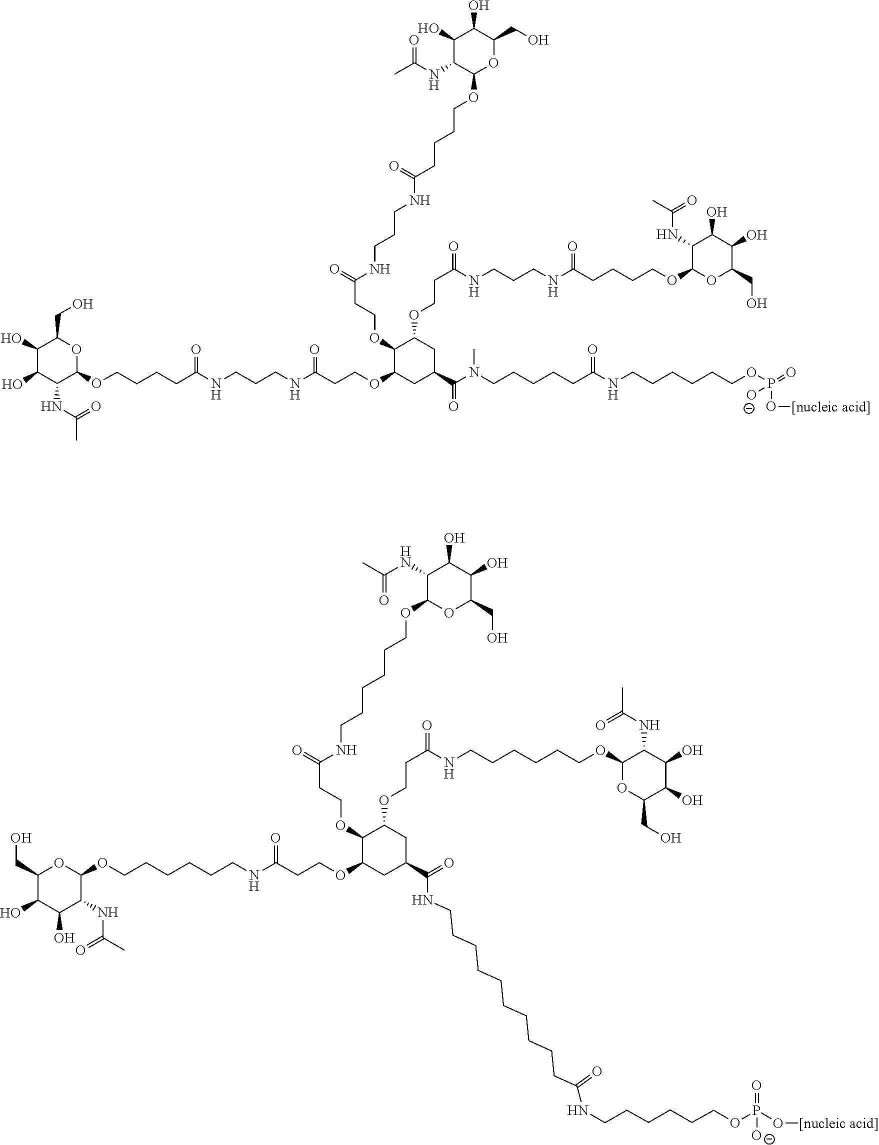

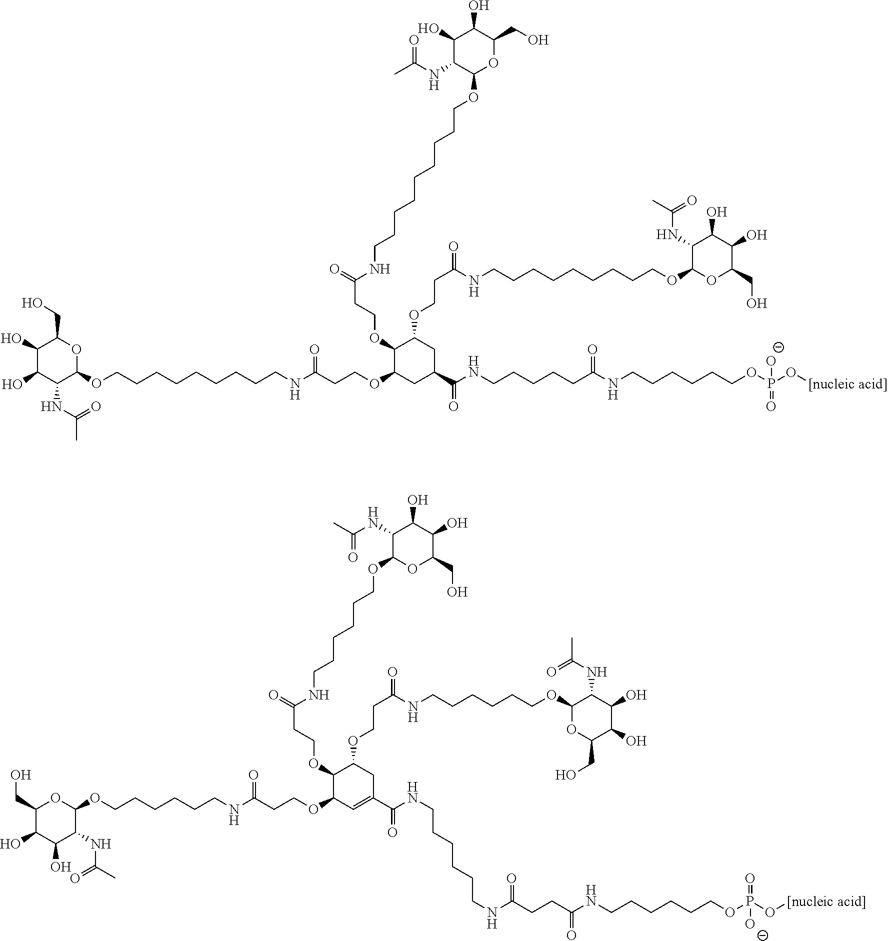

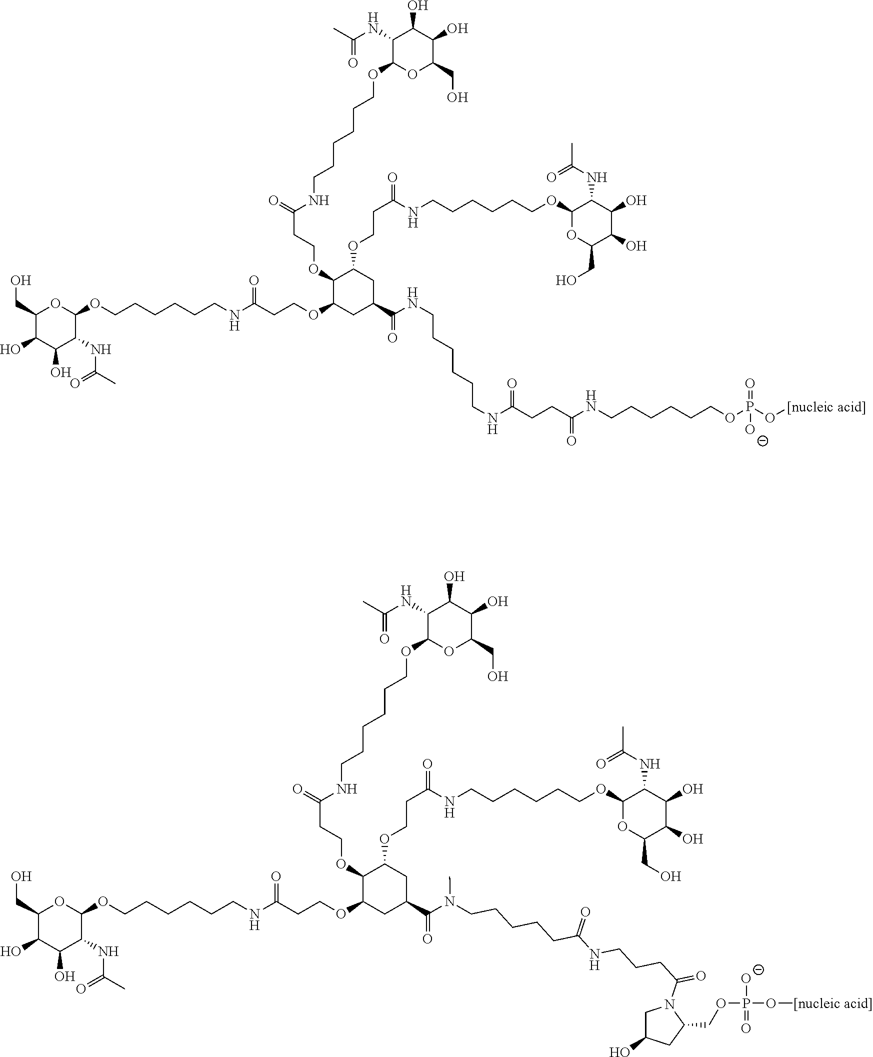

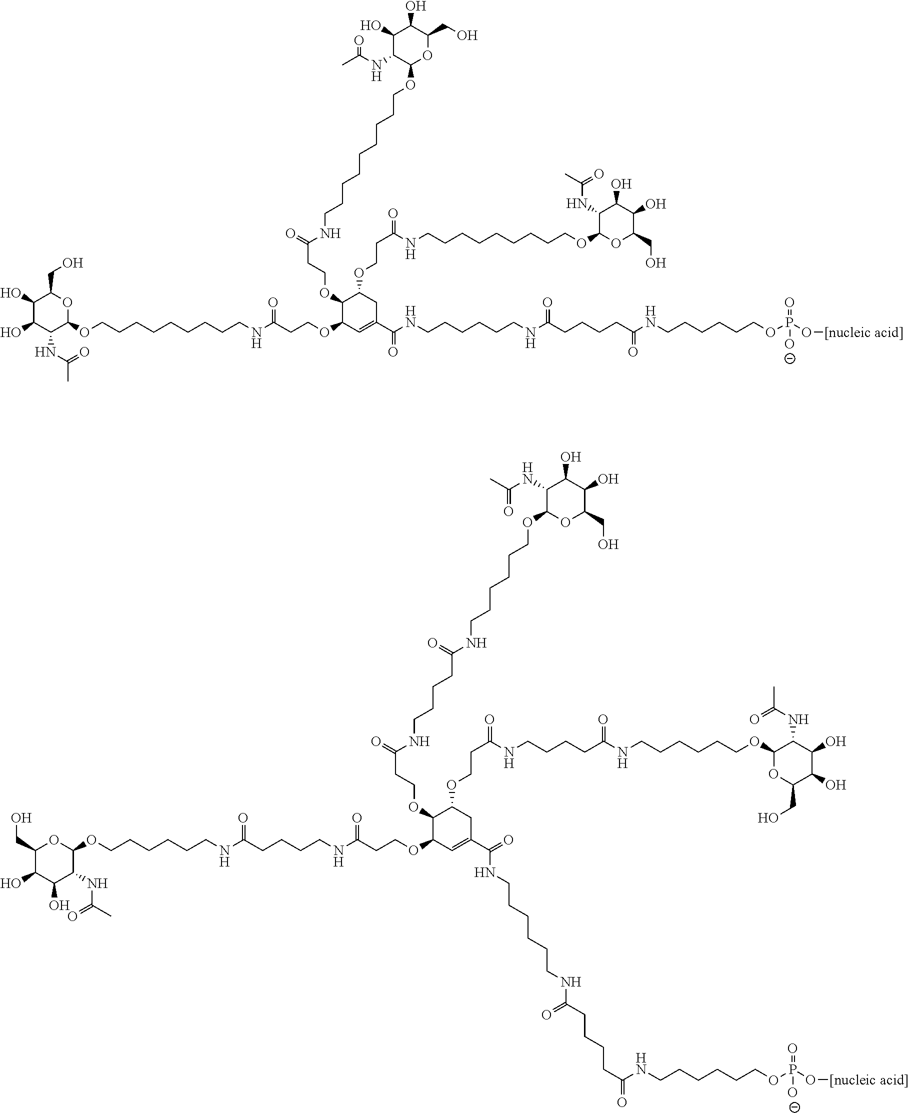

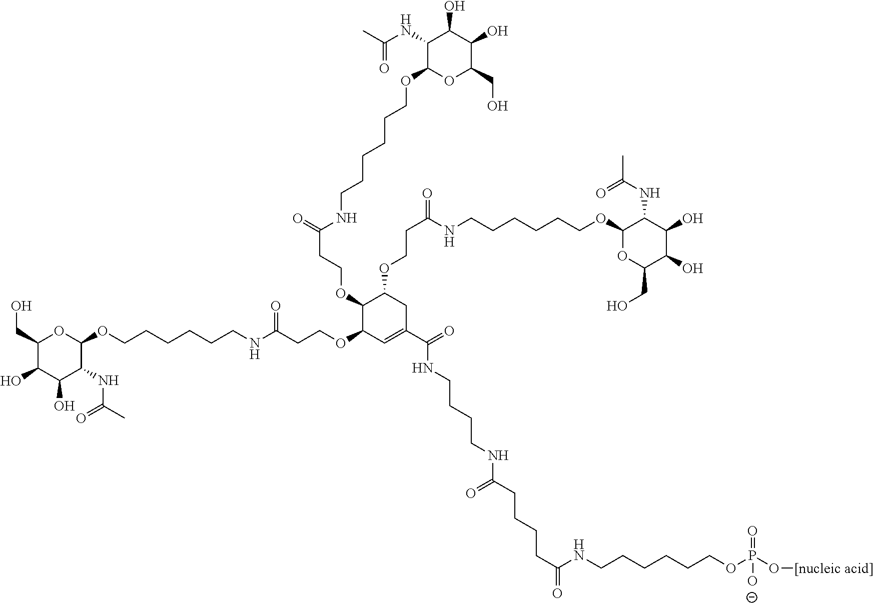

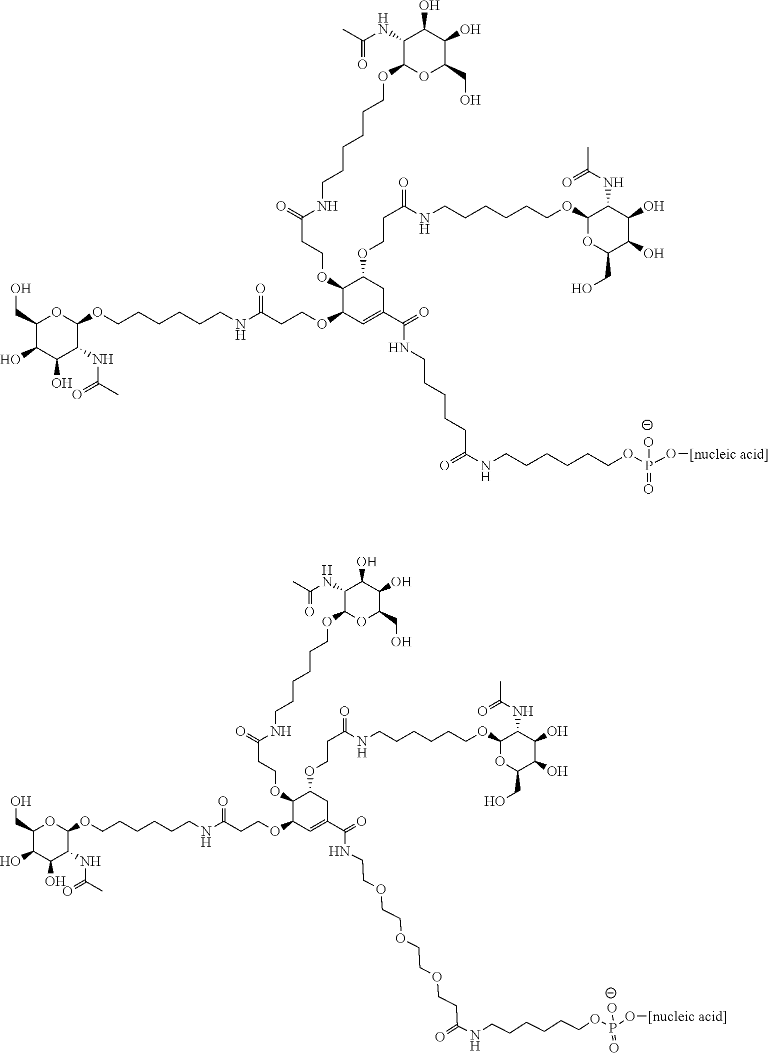

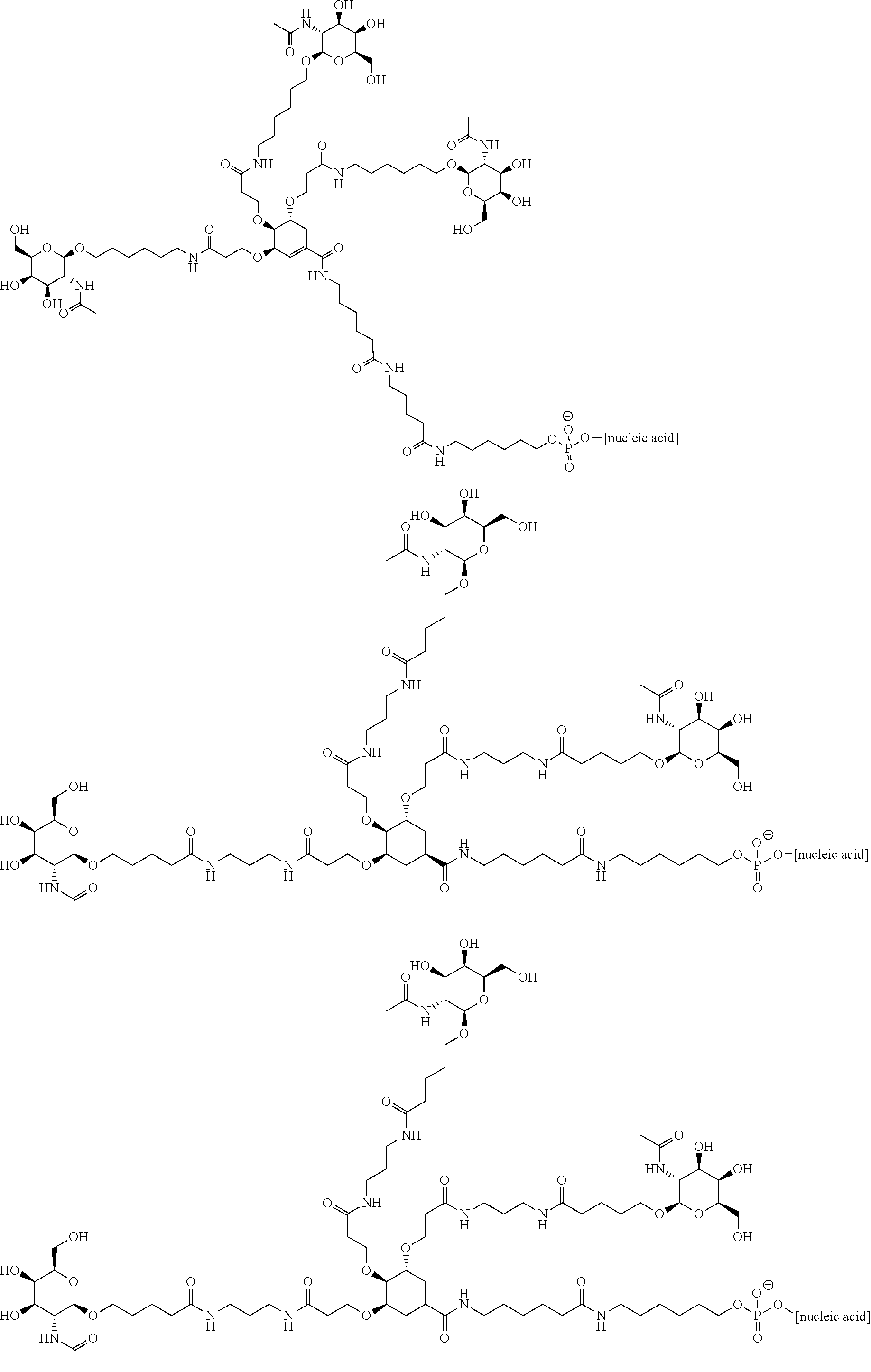

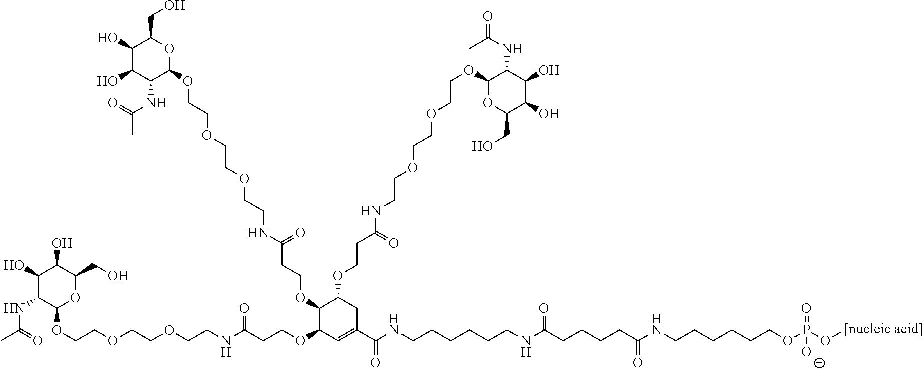

and the pharmaceutically acceptable salts thereof, wherein nucleic acid denotes a cargo as defined in any of the preceding clauses.

32. The compound or pharmaceutically acceptable salt thereof of clause 31, wherein nucleic acid denotes an ASO or siRNA.

33. A compound as defined in any one of clauses 1-32, or a pharmaceutically acceptable salt thereof, having a binding affinity for ASGPR which is characterised by an IC₅₀ value of less than about 20 nM (e.g., an IC₅₀ value of less than about 10 nM, 9 nM, 8 nM, 7 nM, 6 nM, 5 nM, 4 nM, 3 nM, or 2 nM).

34. A compound as defined in any one of clauses 1-32, or a pharmaceutically acceptable salt thereof, having binding kinetics for ASGPR characterised by a Kd value of less than about 8 nM (e.g., a Kd value of less than about 6 nM, 5 nM, 4 nM, 3 nM, 2 nM, 1 nM, or 0.5 nM).

35. A compound as defined in any one of clauses 1-32, or a pharmaceutically acceptable salt thereof, having an activity in knocking down gene expression in HEK293 cells which is characterised by an IC₅₀ value of less than about 50 nM (e.g., an IC₅₀ value of less than about 20 nM, 10 nM, or 5 nM).

36. A compound as defined in any one of clauses 1-32, or a pharmaceutically acceptable salt thereof, having an activity in knocking down gene expression in tissues such as liver in vivo, wherein the compound can knockdown levels of the target mRNA in said tissue by at least about 40% (e.g., by at least about 45%, 50%, 55%, 60%, 65%, or 70%).

37. A pharmaceutical composition comprising a compound as defined in any one of clauses 1-36, or a pharmaceutically acceptable salt thereof, and at least one pharmaceutically acceptable excipient or carrier.

38. A compound or pharmaceutically acceptable salt thereof according to any one of clauses 1-36, or a pharmaceutical composition according to clause 37, for use in therapy.

39. A compound or pharmaceutically acceptable salt thereof according to any one of clauses 1-36, or a pharmaceutical composition according to clause 37, for use in the treatment of a condition selected from liver disease (e.g., liver cancer), genetic disease, haemophilia and bleeding disorders, liver fibrosis, non-alcoholic steatohepatitis (NASH), non-alcoholic fatty liver disease (NAFLD), viral hepatitis, rare diseases (e.g. acromegaly), metabolic disease, cardiovascular disease, obesity, thalassemia, liver injury (e.g., drug induced liver injury), hemochromatosis, alcoholic liver disease, alcohol dependence, anaemia, and anaemia of chronic disease.

40. The compound, pharmaceutically acceptable salt or pharmaceutical composition for use of clause 39, wherein the condition is selected from NASH, NAFLD, a metabolic disease and a cardiovascular disease.

41. The compound, pharmaceutically acceptable salt or pharmaceutical composition for use of clause 39, wherein the condition is NASH.

42. The compound, pharmaceutically acceptable salt or pharmaceutical composition for use of clause 39, wherein the condition is a metabolic disease selected from hypercholesterolemia, dyslipidaemia, and hypertriglyceridemia.

43. A compound having the structure of Formula (VI):

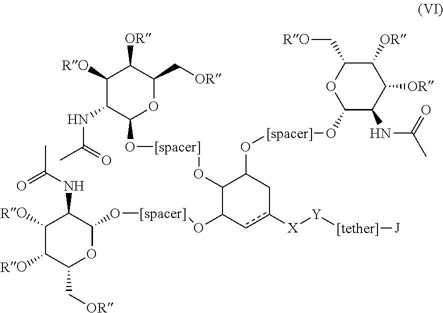

or a pharmaceutically acceptable salt thereof, wherein X, Y, spacer and tether are as defined in any of the preceding clauses; [symbol] represents a carbon-carbon single bond or double bond, with the proviso that when X is a covalent bond [symbol] represents a carbon-carbon single bond; R″ is acyl, —C(O)aryl or —H; and J is —CO₂H, —OH, —C(O)O-(pentafluorophenyl), —N₃ or a phosphoramidite.

44. A process for the preparation of a compound of Formula (III) as defined in any one of clauses 16-19, characterised in that a compound of Formula (VI) as defined in clause 43 is reacted with a compound having the structure

Q-[cargo]

wherein cargo is as defined in any of the preceding clauses, and Q denotes a group which is reactive to group J as defined in clause 43.

45. The process of clause 44, wherein Q denotes a BCN-containing group and J is —N₃.

46. A compound having the structure of Formula (X):

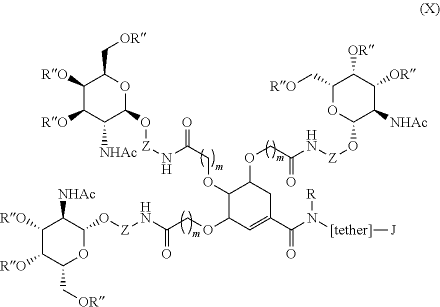

or a pharmaceutically acceptable salt thereof, wherein Z, R, m, and tether are as defined in any of the preceding clauses; R″ is acyl, —C(O)aryl or —H; and J is —CO₂H, —OH, —C(O)O-(pentafluorophenyl), —N₃ or a phosphoramidite.

47. A process for the preparation of a compound of Formula (IX) as defined in clause 29, characterised in that a compound of Formula (X) as defined in clause 46 is reacted with a compound having the structure

Q-[cargo]

wherein cargo is as defined in any of the preceding clauses, and Q denotes a group which is reactive to group J as defined in clause 46.

48. A compound having the structure of Formula (XII):

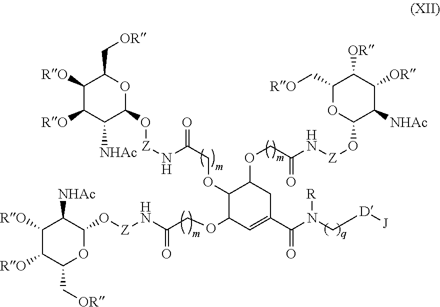

or a pharmaceutically acceptable salt thereof, wherein Z, R, m, and q are as defined in any of the preceding clauses; D′ is selected from (i) a bond, and (ii) —NHC(O)—(CH₂)ₜ— (wherein t is as described herein), R″ is acyl, —C(O)aryl or —H; and J is —CO₂H, —OH, —C(O)O-(pentafluorophenyl), —N₃ or a phosphoramidite.

49. The compound of clause 48, wherein J is —CO₂H or —C(O)O-(pentafluorophenyl).

50. A process for the preparation of a compound of Formula (XI) as defined in clause 30, characterised in that a compound of Formula (XII) as defined in clause 48 or clause 49 is reacted with a compound having the structure

Q-[cargo]

wherein cargo is as defined in any of the preceding clauses, and Q denotes a group which is reactive to group J as defined in clause 48 or clause 49.

### BRIEF DESCRIPTION OF THE FIGURES

FIG. 1 shows a schematic example of compounds of the disclosure. The cargo nucleic acid is attached to the rest of the molecule via a linker and tether. The splitter moiety is bound to the tether on one side and to multiple ligands on the other, in each case with a spacer in between the ligand and the splitter. The complex shown in the figure carries three ligands, but other numbers of ligands are possible (e.g., by employing different splitters). In operation, the ligands (which may be, e.g., GalNAc moieties) typically bind to a receptor or other biomolecule which is associated with a cell or tissue; this facilitates association with and/or uptake into the cell or tissue where the cargo (e.g., a siRNA) can exert a biological effect.

FIG. 2 shows the results of an in vivo assay assessing knockdown of MALAT-1 mRNA in liver cells after a single dose of 50 nmol/kg (Example 3). The relative level of MALAT-1 mRNA in liver is shown compared to the PBS control (as 100%, left-hand column). The middle column shows mRNA levels after treatment with a naked MALAT-1 ASO. The right-hand column shows mRNA levels after treatment with Compound 1 of Example 1.

FIG. 3 shows the pharmacokinetics of ASO exposure after a single dose of 50 nmol/kg (Example 3). The left-hand column shows exposure in liver (nmol/kg) after treatment with a naked MALAT-1 ASO. The right-hand column exposure in liver after treatment with Compound 1 of Example 1.

FIG. 4 shows the results of an in vivo assay assessing knockdown of MALAT-1 mRNA in kidney cells after a single dose of 50 nmol/kg (Example 3). The relative level of MALAT-1 mRNA in kidney is shown compared to the PBS control (as 100%, left-hand column). The middle column shows mRNA levels after treatment with a naked MALAT-1 ASO. The right-hand column shows mRNA levels after treatment with Compound 1 of Example 1.

FIG. 5 shows the pharmacokinetics of ASO exposure after a single dose of 50 nmol/kg (Example 3). The left-hand column shows exposure in kidney (nmol/kg) after treatment with a naked MALAT-1 ASO. The right-hand column exposure in kidney after treatment with Compound 1 of Example 1.

FIG. 6 shows the ANGPTL3 protein concentration measured pre-subcutaneous injection in the in vivo assay of Example 3. The level of ANGPTL3 in ng/ml is shown for the PBS control (column 1, left side), the Control siRNA cluster of Example 1 (column 2, middle), and Compound 2 (column 3, right side). No statistical significance is observed between groups.

FIG. 7 shows the ANGPTL3 protein concentration in plasma at Day 28 (Example 3). Column 1 (left hand side) shows results after administration of PBS control. Columns 2 and 3 show exposure after treatment with the Control siRNA cluster of Example 1 and Compound 2, respectively. Two way ANOVA is used for statistical analysis.

FIG. 8 shows the ANGPTL3 protein concentration in plasma corrected to PBS control over time (Example 3). The black line and circles (top) show the PBS control values. The light grey line and circles (middle) show the results for the Control siRNA cluster of Example 1, and the dark grey line and circles (bottom) show the results for Compound 2.

FIG. 9 shows hepatic Angpt/3 mRNA levels from the in vivo study of Example 3. Values are normalised to PBS control (column 1, left side). Columns 2 and 3 show relative exposure after treatment with the Control siRNA cluster of Example 1 and with Compound 2, respectively.

FIG. 10 shows the relative levels of MALAT1 mRNA in liver (as compared to RPS16 mRNA) from the in vivo study of Example 3. Values are normalised to PBS control (Column 1, 100%). Column 2 shows the knock-down of MALAT1 using the Control ASO cluster of Example 1 (reference), while columns 3-8 show knock-down using Compounds 1, 2c, 3, 5, 6 and 7, respectively.

FIG. 11 shows the relative levels of PPIB mRNA in liver (as compared to RPS16 mRNA) from the in vivo study of Example 3. Values are normalised to PBS control (Column 1, 100%). Column 2 shows the knock-down of PPIB using the Control ASO cluster of Example 1 (reference), while columns 3 and 4 show knock-down using Compounds 2b and 12, respectively.

FIG. 12 shows the relative levels of MALAT1 mRNA in liver (as compared to RPS16 mRNA) from the in vivo study of Example 3. Values are normalised to PBS control (Column 1, 100%). Column 2 shows the knock-down of MALAT1 using the Control ASO cluster of Example 1 (reference), while columns 3-6 show knock-down using Compounds 8-11, respectively.

FIG. 13 shows the relative levels of MALAT1 mRNA in liver (FIG. 13A) and kidney (FIG. 13B) from the in vivo study of Example 3. Values are normalised to PBS control (Column 1, 1.0 in each case). Columns 2-4 shows the knock-down of MALAT1 using Compounds 2c, 16a and 15a, respectively.

### DETAILED DESCRIPTION

Although specific embodiments of the present disclosure will now be described with reference to the description and examples, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of chemical synthesis, tissue culture, immunology, molecular biology, microbiology, cell biology, recombinant DNA, etc., which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (TRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular forms “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, without excluding other elements. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

“GalNAc” refers to 2-(acetylamino)-2-deoxy-D-galactopyranose, commonly referred to in the literature as N-acetyl galactosamine. Reference to “GalNAc” or “N-acetyl galactosamine” herein denotes the R form, i.e., 2-(acetylamino)-2-deoxy-β-D-galactopyranose.

The term “cargo” as used herein refers to a chemical or biological entity which is suitable for targeting to and/or delivery to a cell or tissue, e.g. as part of a compound of the disclosure.

The cargo in accordance with the present disclosure is a nucleic acid.

The term “nucleic acid” as used herein includes nucleic acids selected from the group consisting of DNA, RNA, peptide nucleic acid (PNA), and locked nucleic acid (LNA). The nucleic acid may be a functional nucleic acid, e.g., whereby the functional nucleic acid is selected from the group consisting of mRNA, micro-RNA, shRNA, combinations of RNA and DNA, siRNA, siNA, antisense nucleic acid (e.g., antisense oligonucleotide (ASO)), ribozymes, aptamers and spiegelmers. A “peptide nucleic acid” is a polymer which is similar to DNA or RNA in which the backbone is composed of repeating amino acid (typically N-(2-aminoethyl)-glycine) units linked by peptide bonds. The various purine and pyrimidine bases are linked to the backbone by a methylene bridge and a carbonyl group. A “locked nucleic acid” is a nucleic acid in which the 2′ hydroxyl is connected, e.g., by a methylene bridge, to the 4′ carbon of the same ribose sugar.

By “duplex region” is meant the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 nucleotides on each strand are complementary or substantially complementary, such that the “duplex region” consists of 19 base pairs. The remaining base pairs may exist as 5′ and 3′ overhangs, or as single stranded regions. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well known in the art. Alternatively, two strands can be synthesised and added together under biological conditions to determine if they anneal to one another. The portion of the first strand and second strand that form at least one duplex region may be fully complementary and are at least partially complementary to each other.

The term “abasic” as used herein in connection with nucleotides, refers to moieties lacking a base or having other chemical groups in place of a base at the 1′ position, for example a 3′,3′-linked or 5′,5′-linked deoxyabasic ribose derivative.

The term “alternating” as used herein in connection with nucleic acids means to occur one after another in a regular way. In other words, alternating means to occur in turn repeatedly. For example if one nucleotide is modified, the next contiguous nucleotide is not modified and the following contiguous nucleotide is modified and so on.

As used herein, the term “inhibit”, “down-regulate”, or “reduce” with respect to gene expression means the expression of the gene, or level of RNA molecules or equivalent RNA molecules encoding one or more proteins or protein subunits (e.g., mRNA), or activity of one or more proteins or protein subunits, is reduced below that observed in the absence of a nucleic acid of the disclosure; for example the expression may be reduced to 90%, 80%, 70%, 60%, 50%, 40%, 30%, 20%, 10%, 5% or less than that observed in the absence of an inhibitor.

An “overhang” as used herein has its normal and customary meaning in the art, i.e. a single stranded portion of a nucleic acid that extends beyond the terminal nucleotide of a complementary strand in a double strand nucleic acid. The term “blunt end” includes double stranded nucleic acid whereby both strands terminate at the same position, regardless of whether the terminal nucleotide(s) are base paired.

A “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions of the disclosure is contemplated.

A “subject,” “individual”, or “patient” is used interchangeably herein, and typically refers to a vertebrate, such as a mammal. Mammals include, but are not limited to, rodents, farm animals, sport animals, pets, and primates; for example murines, rats, rabbit, simians, bovines, ovines, porcines, canines, felines, equines, and humans. In a particular embodiment, the mammal is a human.

“Administering” is defined herein as a means of providing an agent or a composition containing the agent to a subject in a manner that results in the agent being contacted with (e.g., being inside) the subject's body. Such an administration can be by any route including, without limitation, oral, transdermal (e.g., by the vagina, rectum, or oral mucosa), by injection (e.g., subcutaneous, intravenous, parenteral, intraperitoneal, or into the central nervous system), or by inhalation (e.g., oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms. A patient or individual may be predisposed to the disease because of the presence of genetic mutations associated with the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including, for example, the activity of the specific compound employed, the age, body weight, general health, sex, and diet of the subject, the time of administration, the rate of excretion, the drug combination, the severity of the particular disorder being treated and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with the condition. The total daily dose may be administered in single or divided doses and may, at the physician's discretion, fall outside of the typical range given herein.

The term “delivering” when used in connection with the nucleic acid-containing compounds and compositions of the disclosure typically denotes some active targeting of the nucleic acid to the target cell or tissue. Thus, delivery of a nucleic acid using the compounds and compositions of the disclosure typically results in exposure of the target cell or tissue to the nucleic acid at a level which is greater than the exposure following administration of the same nucleic acid without the rest of the complex (e.g., when administered as ‘naked’ nucleic acid).

As used herein, the term “pharmaceutically acceptable excipient” or “pharmaceutically acceptable carrier” encompasses any of the standard pharmaceutical ingredients, for example as described in Remington's Pharmaceutical Sciences (20^{th} ed., Mack Publishing Co. 2000). Such excipients include carriers such as a phosphate buffered saline solution, water, and emulsions, such as an oil/water or water/oil emulsion, and various types of wetting agents. Pharmaceutical compositions also can include stabilizers, preservatives, adjuvants, fillers, binders, lubricants, and the like.

As used herein, the term “alkyl” means a saturated linear or branched free radical consisting essentially of carbon atoms and a corresponding number of hydrogen atoms. The term “alkylene” has the corresponding meaning in connection with the divalent free radical. Exemplary alkyl groups include methyl, ethyl, n-propyl, isopropyl, n-butyl, isobutyl, etc. Other alkyl groups will be readily apparent to those of skill in the art given the benefit of the present disclosure. The terms “(C₁-C₃)alkyl”, “(C₁-C₆)alkyl”, etc., have equivalent meanings, i.e., a saturated linear or branched free radical consisting essentially of 1 to 3 (or 1 to 6) carbon atoms and a corresponding number of hydrogen atoms. The definition of “alkyl” also applies in the context of other groups which comprise alkyl groups, such as “—O(C₁-C₃)alkyl”. The term “haloalkyl” means an alkyl group which is substituted by one or more halogens. Exemplary haloalkyl groups include trifluoromethyl, trifluoroethyl, difluoroethyl, pentafluoroethyl, chloromethyl, etc. One or more carbon atoms in the backbone of the alkyl group may be substituted by (or bonded to) a heteroatom by a multiple bond (e.g., a double bond); for example, a carbon atom of the alkyl group may be bonded to oxygen via a double bond (i.e., substituted by oxo to provide a carbonyl function). The presence of such a substituent does not prevent the carbon backbone of the free radical being considered as an alkyl group. In embodiments, the alkyl group is linear. Alkyl groups of the present disclosure may be substituted with one or more optional substituents as defined herein.

As used herein, the term “alkenyl” means an unsaturated linear or branched free radical consisting essentially of carbon atoms and a corresponding number of hydrogen atoms, which free radical comprises at least one carbon-carbon double bond. The term “alkenylene” has the corresponding meaning in connection with the divalent free radical. Exemplary alkenyl groups include ethenyl, prop-1-enyl, prop-2-enyl, isopropenyl, but-1-enyl, 2-methyl-prop-1-enyl, and 2-methyl-prop-2-enyl. The terms “(C₂-C₆)alkenyl”, etc., have equivalent meanings, i.e., an unsaturated linear or branched free radical consisting essentially of 2 to 6 carbon atoms and a corresponding number of hydrogen atoms. Alkenyl groups of the present disclosure may be substituted with one or more optional substituents as defined herein.

As used herein, the term “aryl” means an aromatic free radical having at least 6 carbon atoms (i.e., ring atoms) that form a ring. It will be appreciated that the aryl group may be monocyclic or multicyclic (e.g., fused). In the case of multicyclic aryl groups, there are further rings, e.g. 1 or more further rings, all of which contain at least 3 carbon atoms (i.e., ring atoms). Examples of aryl groups include phenyl and naphthalenyl. The aryl group may contain from 6 to 10 carbon atoms in the ring portion of the group, which may be monocyclic or multicyclic (e.g., fused). In embodiments, aryl is phenyl. Aryl groups of the present disclosure may be substituted with one or more optional substituents as defined herein.

As used herein, the term “cycloalkyl” means a saturated free radical having at least 3 to 9 carbon atoms (i.e., ring atoms) that form a ring. Exemplary cycloalkyl groups include cyclopropyl, cyclobutyl, cyclopentyl and cyclohexyl. It will be appreciated that the cycloalkyl group may be monocyclic or multicyclic (e.g., fused, bridged, or spirocyclic). In the case of multicyclic cycloalkyl groups, there are further rings, e.g. 1 or more further rings, all of which contain from 3 to 7 carbon atoms (i.e., ring atoms). Exemplary cycloalkyl groups having such further rings include bicyclo[1.1.1]pentanyl. One or more ring atoms of the cycloalkyl group may be substituted by (i.e., bonded to) a heteroatom by a double bond (e.g., cycloalkyl substituted by oxo). The presence of such a substituent does not prevent the carbon backbone of the free radical being considered as a cycloalkyl group. Cycloalkyl groups of the present disclosure may be substituted with one or more optional substituents as defined herein.

As used herein, the term “heterocycloalkyl” means a saturated free radical having at least 3 to 10 atoms (i.e., ring atoms) that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen. Heterocycloalkyl rings may have oxo substituents, typically adjacent to a heteroatom (e.g., 2-oxopyrrolidinyl), but the oxygen atom does not form part of the ring and is excluded from the number of ring atoms. The presence of such a substituent does not prevent the ring (or rings) of the free radical being considered as a heterocycloalkyl group. Exemplary heterocycloalkyl groups include tetrahydrofuranyl, piperidinyl, morpholinyl and piperazinyl. In the case of multicyclic heterocyclic groups, there are further rings, e.g. 1 or more further rings, all of which contain from 3 to 7 ring atoms selected from carbon, nitrogen, sulphur, and oxygen. The further rings may be saturated, or partially or fully unsaturated (e.g., having aromatic character). Multicyclic heterocyclic groups include fused, bridged and spirocyclic ring systems. Where a multicyclic heterocycloalkyl group contains an unsaturated fused ring, the group is typically not bonded to the rest of the molecule via that fused ring. Heterocycloalkyl groups of the present disclosure may be substituted with one or more optional substituents as defined herein.

The term “phosphate” is typically used herein to denote a radical (or diradical) group which comprises a central phosphorus atom bonded to four oxygen atoms (one via a double bond). The oxygen atoms which are bonded to phosphorus via single bonds may represent points of attachment to the rest of the molecule, or may carry hydrogen atoms, or may be considered to carry a negative charge (e.g., in the case of salts). The term “thiophosphate” is typically used herein to denote a phosphate analogue in which at least one oxygen atom is replaced by sulphur; such groups are also called “phosphorothioates” herein.

An “optional substituent” is a group which is covalently attached to a moiety (generally via a carbon atom of the moiety, and typically in place of a hydrogen atom on said carbon atom). The optional substituent may be chosen to be a group which does not significantly alter the steric and/or electronic properties of the molecule. In embodiments, each optional substituent is independently selected from the group consisting of: halogen (e.g., —F, —Cl, —Br or —I); —OH; —SH; —NH₂; —NHMe; —NMe₂; —(C₁-C₃)alkyl (e.g., -Me or -Et); and 3- or 4-membered cycloalkyl or heterocycloalkyl group (e.g., cyclopropyl or epoxide), which may optionally be substituted with one or more halo. A group defined as “optionally substituted” may be either unsubstituted, or substituted with one or more substituents, e.g. 1, 2, 3, 4, 5, 6, or more substituents. In embodiments, a substituted group has 1 to 4 substituents, e.g. 1, 2, or 3 substituents. In embodiments, a substituted group has 1 or 2 substituents. In embodiments, a substituted group has 3 substituents.

As used herein, the terms “halo” and “halogen” mean fluorine, chlorine, bromine, or iodine. These terms are used interchangeably and may refer to a halogen free radical group or to a halogen atom as such. Those of skill in the art will readily be able to ascertain the identification of which in view of the context in which this term is used in the present disclosure. In embodiments, the halogen is fluorine.

The compounds of the present disclosure are described, inter alia, by way of structural formulae. It will be appreciated that these formulae typically show only one form (e.g., resonance form, tautomeric form, etc.) of the compound, whereas certain compounds may exist in more than one such form. This will be readily apparent to the skilled reader. The present disclosure includes all possible tautomers of the compounds characterised by the structural formulae herein, including as single tautomers, or as any mixture of tautomers in any ratio. It will also be appreciated that certain of the present compounds may exist in one or more isomeric (e.g., stereoisomeric) forms. The present disclosure includes all possible stereoisomers, enantiomers, diastereomers, etc. of the compounds described hereinbefore and below, as well as cis- and trans- forms and conformers of the same. The purification and the separation of isomers may be accomplished by methods described hereinafter, as well as by techniques known in the art. For example, optical isomers of the compounds can be obtained by resolution of the racemic mixture of diastereoisomeric salts thereof (e.g., using an optically active acid or base, or by the formation of covalent diastereomers). A different process for separation of optical isomers involves the use of chiral chromatography (e.g., HPLC columns using a chiral phase), with or without conventional derivatization. Enzymatic separation, with or without derivatisation, may also be useful, and optically active compounds of the present disclosure can likewise be obtained by chiral syntheses utilizing optically active starting materials. The present disclosure includes all possible stereoisomers of the compounds described herein as single stereoisomers, or as any mixture of said stereoisomers, e.g. (R)- or (S)- isomers, in any ratio.

The compounds of the disclosure may exist in the form of free acids or bases, or may exist as addition salts with suitable acids or bases. Methods for forming salts are described below and are also known in the art (see, e.g., Berge et al., J Pharm Sci. (1977) 66:1-19). As used herein, the term “pharmaceutically acceptable” when used in connection with salts means a salt of a currently disclosed compound that may be administered without any resultant substantial undesirable biological effect(s) or any resultant deleterious interaction(s) with any other component of a pharmaceutical composition in which it may be contained.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Compounds, compositions and methods provided herein may be combined with one or more of any of the other compounds, compositions and methods provided herein.

The following abbreviations and empirical formulae are used herein:
Ac acetyl
ACN acetonitrile
ANGPTL3 angiopoietin-like 3
anti-miR anti-microRNA
Appp 5′-adenosine cap
ASGPR asialoglycoprotein receptor
ASO antisense oligonucleotide
BCN bicyclo[6.1.0]non-4-yne group
BOP benzotriazole-1-yl-oxy-tris-(dimethylamino)
Cbz carboxybenzyl
cDNA complementary DNA
cEt constrained ethyl
CRISPR clustered regularly interspaced short palindromic repeats
dA deoxyadenosine
dC deoxycytidine
DCE dichloroethane
DCM dichloromethane
dG 2′-deoxyguanosine
DIEA diethylamine
DMAP 4-dimethylaminopyridine
DMEM Dulbecco's modified eagle medium
DMSO dimethylsulfoxide
DMTr 4,4′-dimethoxytrityl
DNA deoxyribonucleic acid
Ds 7-(2-thienyl)-imidazo[4,5-b]pyridine
dT thymidine
EDC 1-ethyl-3-(3-dimethylaminopropyl)carbodiimide hydrochloride
ELISA enzyme-linked immunosorbent assay
ESI electrospray ionization
GalNAc 2-(acetylamino)-2-deoxy-D-galactopyranose
HBTU N,N,N′,N′-Tetramethyl-O-(1H-benzotriazol-1-yl)uronium hexafluorophosphate
HEK293 human embryonic kidney 293
HPLC high performance liquid chromatography
invAb inverted abasic deoxyribose
LCMS liquid chromatography mass spectrometry
LNA locked nucleic acid
m/z mass to charge ratio
m6A N6-methyladenosine
MALAT1 metastasis associated lung adenocarcinoma transcript 1
mC 5′-methylcytidine
MMT 5′-monomethoxytrityl
MOE methoxyethyl
MPEG poly(ethylene glycol) methyl ether
MPS mononuclear phagocyte system
mQ Milli-Q®
mRNA messenger RNA
MS mass spectrometry
NAFLD non-alcoholic fatty liver disease
NASH non-alcoholic steatohepatitis
Pa pyrrole 2-carbaldehyde
PBS phosphate-buffered saline
PCR polymerase chain reaction
PEG polyethyleneglycol
PFP pentafluorophenyl
PNA peptide nucleic acid
PO phosphate
pol III RNA polymerase III
PHH primary human hepatocytes
PPIB peptidyl-prolyl cis-trans isomerase B
PS thiophosphate (phosphorothioate)
qPCR real-time polymerase chain reaction
RES reticuloendothelial system
RISC RNA-induced silencing complex
RNA ribonucleic acid
RNAi RNA interference
saRNA small activating ribonucleic acid
SEQ ID sequence identifier
shRNA short hairpin RNA
siNA small interfering nucleic acid
siRNA small interfering RNA
TAMRA carboxytetramethylrhodamine
TBAF tetrabutylammonium fluoride
TBS tert-butyldimethylsilyl
TFA trifluoroacetic acid
THE tetrahydrofuran
TMS-OTf Trimethylsilyl trifluoromethanesulfonate (also TMs-OTf)
TMS-I Trimethylsilyl iodide
TOF time of flight
Tos toluenesulfonyl
TPPC4 trafficking protein particle complex subunit 4
U uracil
UV ultraviolet

### Compounds

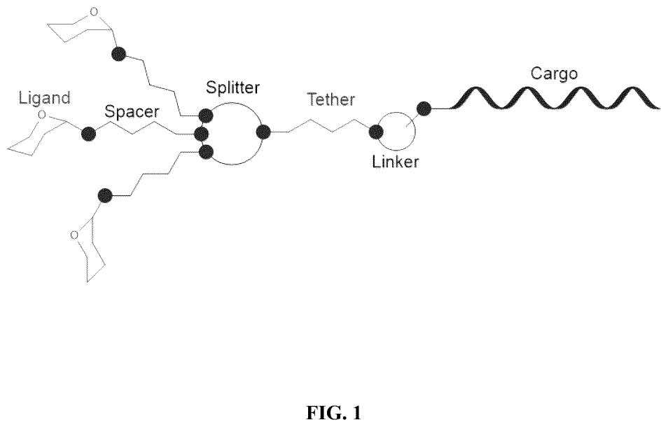

The present disclosure provides monosaccharide-containing compounds which are useful, inter alia, for targeting nucleic acid cargo moieties to specific locations (e.g., cell and/or tissue types) in vivo. As illustrated schematically in FIG. 1, the compounds have several ‘arms’ which carry the targeting ligands and cargo moieties. These, in turn, are typically bound together by a ‘splitter’ moiety (also called a “branching unit”), which has the appropriate functionality to join the arms of the compound together. The present disclosure relates, in particular, to compounds in which the splitter is derived from, or chemically related to, shikimic acid, in which the ligand-containing arms may be attached to the hydroxyl groups on the cyclohexyl ring. The use of shikimic acid and analogues thereof in preparing the branching unit provides a novel way of coupling targeting and cargo moieties which may have advantages over known methods, including for example the use of simple, natural precursors. The compounds of the invention exhibit useful properties such as, e.g. improved targeting, increased activity and/or improved pharmacokinetic properties in vivo.

Thus, viewed from a first aspect the present disclosure provides a compound, or a pharmaceutically acceptable salt thereof, the compound having the structure of Formula (I):

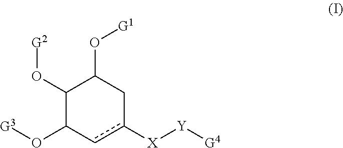

wherein:
X is selected from a covalent bond, —CH₂—, and —C(O)—;
Y is —O— or —NR—,
   wherein R is selected from —H and —(C₁-C₆)alkyl;
[symbol] represents a carbon-carbon single bond or double bond, with the proviso that when X is a covalent bond [symbol] represents a carbon-carbon single bond; and
G¹ to G⁴ each independently represents a -[spacer]-[ligand] moiety or a -[tether]-[linker]-[cargo] moiety, wherein each ligand is independently a monosaccharide and each cargo is independently a nucleic acid, with the proviso that at least one of G¹ to G⁴ represents a -[spacer]-[ligand] moiety and at least one of G¹ to G⁴ represents a -[tether]-[linker]-[cargo] moiety.

In embodiments, X is —C(O)—. In other embodiments, X is a covalent bond.

In embodiments, Y is —NR—, e.g., wherein R is selected from —H and —(C₁-C₃)alkyl. In embodiments, R is —H. In embodiments, R is —CH₃. In embodiments, Y is —NH—. In embodiments, Y is —N(CH₃)—.

In embodiments, X is —C(O)— and Y is —NR—, e.g., wherein R is selected from —H and —(C₁-C₃)alkyl. In embodiments, X is —C(O)— and Y is —NH—. In embodiments, X is —C(O)— and Y is —N(CH₃)—. In other embodiments, X is a covalent bond and Y is —NR—, e.g., wherein R is selected from —H and —(C₁-C₃)alkyl.

In embodiments, [symbol] represents a single bond. In other embodiments, =represents a double bond.

In embodiments, the compound is a compound having the structure of Formula (Ia):

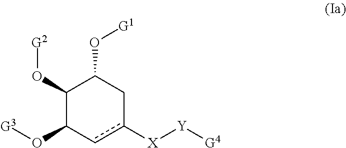

or a pharmaceutically acceptable salt thereof, wherein X, Y, G¹, G², G³ and G⁴ are as defined herein.

In embodiments, the compound is a compound having the structure of Formula (Ib):

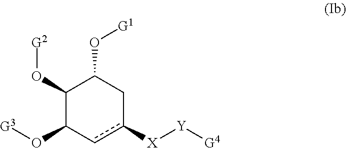

or a pharmaceutically acceptable salt thereof, wherein X, Y, G¹, G², G³ and G⁴ are as defined herein, and [symbol] represents a single bond.

In other embodiments, the compound is a compound having the structure of Formula (Ic):

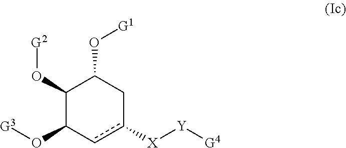

or a pharmaceutically acceptable salt thereof, wherein X, Y, G¹, G², G³ and G⁴ are as defined herein, and [symbol] represents a single bond.

In other embodiments, the compound is a compound having the structure of Formula (Id):

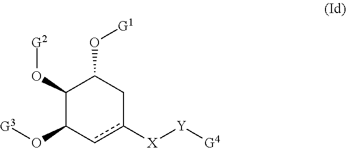

or a pharmaceutically acceptable salt thereof, wherein X, Y, G¹, G², G³ and G⁴ are as defined herein, and [symbol] represents a double bond.

Each “ligand” which is present in the compounds of the disclosure is a monosaccharide. The monosaccharide may be selected to have an affinity for the hepatic asialoglycoprotein receptor (ASGPR). In embodiments, the target ASGPR is on the surface of a mammalian liver cell. In embodiments, the ligand is GalNAc. In embodiments, the compounds of the disclosure have 2, 3 or 4 (e.g., 2 or 3) ligands which are all GalNAc.

The “tether” and “linker” groups taken together act to join the cargo moiety (or moieties) to the rest of the molecule, i.e. to the splitter. Suitable tethers and linkers will be apparent to the skilled person based on the current description and its Examples. In embodiments, the [tether]-[linker] moiety is obtained by reacting a tether precursor (which may be bonded to the rest of the molecule, i.e. to the spacer/ligand construct) with a linker precursor (which may be bonded to the cargo) to join the two together. Suitable methods for reacting the tether and linker precursors are described in the following Examples and would be known to the skilled person. Examples of such methods include ‘click’ chemistry reactions such as copper catalysed cycloadditions between an azide and an alkyne (see, e.g., Fantoni et al., Chem. Rev. (2021) 121(12):7122-7154). In other embodiments, the [tether]-[linker] moiety is introduced into the complex without the cargo attached and the cargo is subsequently bound to the linker. The complex may conveniently be coupled with a cargo in a final step of a solid-phase synthesis, e.g. where the [tether]-[linker] moiety is initially introduced with a phosphate group at its distal end that can be reacted with a cargo nucleic acid on solid phase to yield the final compound (see, e.g., Beaucage, Curr Opin Drug Discov Dev (2008) 11(2):203-216). Other examples of tethers and/or linkers which might be used in accordance with the present disclosure are found, e.g., in international patent publications WO 2014/179620, WO 2015/177668, WO 2009/073809, WO 2012/083046, WO 2017/156012, WO 2016/100401, WO 2017/174657 and WO 2019/092280, the contents of each of which are incorporated herein in their entirety.

In embodiments, the tether and linker together represent a moiety comprising a linear chain of 8 to 30 atoms which links the cargo to the rest of the molecule. In embodiments, the tether and linker together represent an optionally substituted linear chain of 8 to 30 atoms selected from C, N, O, S and P, e.g. a chain of 9 to 24 atoms, 10 to 20 atoms, or 12 to 18 atoms. In embodiments, the tether and linker together comprise one or more groups selected from an amide (e.g., obtained by reaction of a carboxylic acid or acid derivative with an amine), an ester (e.g., obtained by reaction of a carboxylic acid or acid derivative with an alcohol, such as an aliphatic or aromatic alcohol), or a 1,2,3-triazole (e.g., obtained by reaction of an azide with an alkyne). In embodiments, the tether and linker together comprise (e.g., consist of) a group represented by:

—(CH₂)_{q}-D-(CH₂)ₛ—*

wherein q is an integer from 1 to 12 (e.g., from 2 to 8, such as 6); D is selected from (i) a direct bond, (ii) —C(O)NH—, (iii) a 1,2,3-triazole containing group, and (iv) —NHC(O)—(CH₂)ₜ—C(O)NH—, wherein t is an integer from 0 to 6 (e.g., from 1 to 4, such as 2); and s is an integer from 0 to 12 (e.g., from 2 to 8, such as 6). The group may be optionally substituted and the * denotes the point of attachment to the cargo. It will be appreciated that the [tether]-[linker] group illustrated above will typically be connected to the cargo via a phosphate group. Thus, in embodiments tether and linker together comprise a group as shown above which further comprises a phosphate group (e.g., —OP(═O)(OH)O—) at the position marked with *. In embodiments, q is an integer from 1 to 12 (e.g., from 2 to 8, such as 6); D is selected from (i) a direct bond, (ii) —C(O)NH—, and (iii) a 1,2,3-triazole containing group; and s is an integer from 0 to 12 (e.g., from 2 to 8, such as 6).

In embodiments, D is a direct bond. The tether and linker together may thus comprise (e.g., consist of) a group represented by —(CH₂)_{q}(CH₂)ₛ—*, wherein q is an integer from 1 to 12 (e.g., 6), and s is an integer from 0 to 12 (e.g., 6). In embodiments, s is 0, in which case the tether and linker together comprise (e.g., consist of) a group represented by —(CH₂)_{q}— wherein q is an integer from 1 to 12 (e.g., 6).

In other embodiments, D is —C(O)NH—. The tether and linker together may thus comprise (e.g., consist of) a group represented by —(CH₂)_{q}—C(O)NH—(CH₂)ₛ—*, wherein q is an integer from 1 to 12 (e.g., 6), and s is an integer from 0 to 12 (e.g., 6). In embodiments, q is 6 and s is 6.

In yet further embodiments, D is a 1,2,3-triazole containing group, e.g. a group which is obtained by the reaction of BCN with an azide. Thus, in embodiments D comprises (e.g., consists of) a group

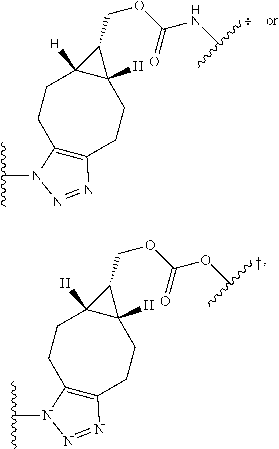 [SMILES: *NC(=O)OC[C@@H]1[C@@H]2CCc3nnn(*)c3CC[C@@H]21 ; *OC(=O)OC[C@@H]1[C@@H]2CCc3nnn(*)c3CC[C@@H]21]

wherein † denotes the point of attachment to —(CH₂)ₛ—. In embodiments, tether and linker together comprise (e.g., consist of) a group represented by

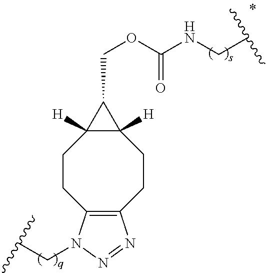

wherein q and s are defined herein (e.g., wherein q is 6 and s is 6).

In yet further embodiments, D is —NHC(O)—(CH₂)—C(O)NH—, and t is an integer from 0 to 6 (e.g., from 1 to 4, such as 2). In embodiments, tether and linker together comprise (e.g., consist of) a group represented by —(CH₂)_{q}—NHC(O)—(CH₂)ₜ-C(O)NH—(CH₂)ₛ—*, wherein q and s are defined herein (e.g., wherein q is 6 and s is 6).

In other embodiments, the tether and linker together comprise (e.g., consist of) a group represented by:

—(CH₂)ᵤ—C(O)NH—(CH₂)ᵥ-E-*

wherein u is an integer from 0 to 11 (e.g., from 1 to 7, such as 5); v is an integer from 1 to 10 (e.g., from 2 to 5, such as 3); and E is a pyrrolidine containing group. The group may be optionally substituted and the * denotes the point of attachment to the cargo. It will be appreciated that the [tether]-[linker] group illustrated above will typically be connected to the cargo via a phosphate group. Thus, in embodiments tether and linker together comprise a group as shown above which further comprises a phosphate group (e.g., —OP(═O)(OH)O—) at the position marked with *.

In embodiments, E comprises (e.g., consists of) a group represented by

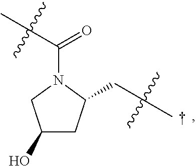 [SMILES: *C[C@@H]1C[C@@H](O)CN1C(*)=O]

wherein † denotes the point of attachment to the cargo. In embodiments, the tether and linker together comprise (e.g., consist of) a group represented by

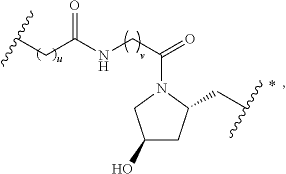

wherein u and v are defined herein (e.g., wherein u is 5 and v is 3).

As will be appreciated, the [tether]-[linker] moiety can conceptually be ‘split’ in a number of ways to yield the individual tether and linker components. In embodiments, the [tether]-[linker] moiety is generated by the reaction of a cargo-containing moiety (that comprises the linker, or a portion thereof) with a ligand-containing precursor (that comprises the tether, or a portion thereof). Thus, in embodiments the tether is represented by a group —(CH₂)_{q}— or —(CH₂)_{q}—C(O)—, wherein q is as defined herein. Likewise, in embodiments the linker is represented by a group -D-(CH₂)ₛ—* or —NH—(CH₂)ₛ—*, wherein D and s are as defined herein. In embodiments, the tether is —(CH₂)_{q}— wherein q is 6, and the linker is -D-(CH₂)ₛ—* wherein D is a direct bond and s is 0. In other embodiments, the tether is —(CH₂)_{q}—C(O)— wherein q is 6, and the linker is —NH—(CH₂)ₛ—* wherein s is 6. In other embodiments, the tether is —(CH₂)_{q}—NH— wherein q is 6, and the linker is —C(O)—(CH₂)ₜ—C(O)NH—(CH₂)ₛ—* wherein t is 2 and s is 6. In other embodiments, the tether is —(CH₂)_{q}—NHC(O)—(CH₂)—C(O)— wherein q is 6 and t is 2, and the linker is —NH—(CH₂)ₛ—* wherein s is 6. In yet further embodiments, the tether is —(CH₂)_{q}— wherein q is 6, and the linker is -D-(CH₂)ₛ—* wherein D is a group

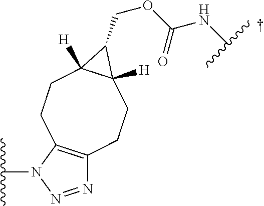 [SMILES: *NC(=O)OC[C@@H]1[C@@H]2CCc3nnn(*)c3CC[C@@H]21]

and s is 6. In still further embodiments, the tether is —(CH₂)ᵤ—C(O)— (e.g., wherein u is 5), and the linker is —NH—(CH₂)ᵥ-E* (e.g., wherein v is 3 and E is a pyrrolidine containing group as defined herein).

The “cargo” groups of the compounds disclosed herein are nucleic acids. These may be single stranded or double stranded. In embodiments, the cargo is an oligonucleotide. In embodiments, the cargo is selected from an antisense oligonucleotide (ASO), an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide and a G-quadruplex. In one embodiment, the cargo is an antisense oligonucleotide (ASO). In another embodiment, the cargo is a siRNA.

The “spacer” comprises a chain of atoms, typically carbon atoms with one or more heteroatoms (e.g. selected independently from N, O, S, and P) optionally intervening that serves to link the ligand to the splitter moiety (i.e., the shikimic acid-derived core). In embodiments, the spacer comprises a chain of 2-20 atoms selected from C, N, O, S and P (e.g., a chain of 7-14 atoms). Exemplary spacers include linear alkylenes (which may optionally be interrupted by one or more amide and/or phosphate groups) and polyethylene glycols. Suitable spacers will be apparent to the skilled person based on the current description and its Examples. Other examples of spacers which might be used in accordance with the present disclosure are found, e.g., in international patent publications WO 2014/179620, WO 2015/177668, WO 2009/073809, WO 2012/083046, WO 2017/156012, WO 2016/100401, WO 2017/174657 and WO 2019/092280, the contents of each of which are incorporated herein in their entirety.

In embodiments, a spacer (e.g., each spacer) has the formula *—Z—NH—C(O)—(CH₂)ₘ-†, wherein: Z in each case independently represents a moiety comprising a linear chain of 1 to 16 atoms (e.g., selected from C, N, O, S and P); and m in each case is independently selected from the integers from 1 to 6, wherein * denotes the point of attachment of the spacer to the ligand and † denotes the point of attachment to the splitter moiety (i.e., the shikimic acid-derived core). In embodiments, * denotes the point of attachment to an oxygen atom of the ligand (e.g., a GalNac oxygen atom).

In embodiments, Z in each case is independently selected from *—(C₁-C₁₆)alkylene-, *—(C₂-C₁₆)alkenylene-, *—(C₁-C₁₂)alkylene-C(O)—, *—(C₂-C₁₂)alkenylene-C(O)—, *—(C₁-C₅)alkylene-C(O)NR′—(C₁-C₆)alkylene-, *—(C₂-C₈)alkenylene-C(O)NR′—(C₁-C₆)alkylene-, *—(C₁-C₈)alkylene-C(O)NR′—(C₂-C₆)alkenylene- and *—(C₂-C₈)alkenylene-C(O)NR′—(C₂-C₆)alkenylene- (where * in each case denotes the point of attachment to the GalNac oxygen atom), wherein R′ in each case is independently selected from —H and —(C₁-C₆)alkyl. In embodiments, Z in each case is independently selected from *—(C₁-C₁₂)alkylene- and *—(C₁-C₆)alkylene-C(O)NR′—(C₁-C₄)alkylene-, wherein R′ in each case is independently selected from —H and —(C₁-C₆)alkyl. In embodiments, each Z is independently selected from *—(C₁-C₁₂)alkylene-, such as *—(C₁-C₁₀)alkylene-, *—(C₂-C₈)alkylene- or *—(C₄-C₇)alkylene-, e.g. wherein Z is n-hexylene. In other embodiments, each Z is independently selected from *—(C₁-C₆)alkylene-C(O)NR′—(C₁-C₄)alkylene- (wherein R′ in each case is independently selected from —H and —(C₁-C₆)alkyl), such as *—(C₂-C₅)alkylene-C(O)NR′—(C₂-C₄)alkylene-, e.g. wherein Z is*—(C₄)alkylene-C(O)NH—(C₃)alkylene-. In embodiments, each Z is the same.

In embodiments, m in each case is independently selected from the integers from 1 to 5. In embodiments, m in each case is independently selected from 1, 2, 3 and 4. In embodiments, m in each case is independently selected from 1, 2, and 3. In embodiments, each m is the same. In embodiments, m is 1. In other embodiments, m is 2. In other embodiments, m is 3.

In embodiments, m is independently selected from the integers from 1 to 4, e.g. from 1 to 3.

In embodiments, m is independently 1 or 2. In embodiments, m is 2. In embodiments, Z is n-hexylene and m is 2.

In embodiments, G¹ to G³ independently represent a -[spacer]-[ligand] moiety (e.g., as defined herein), and G⁴ represents a -[tether]-[linker]-[cargo] moiety (e.g., as described herein). Viewed from this aspect, the disclosure provides a compound, or a pharmaceutically acceptable salt thereof, the compound having the structure of Formula (II):

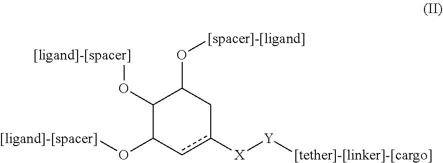

wherein:
X is selected from a covalent bond, —CH₂—, and —C(O)—;
Y is —O— or —NR—,
   wherein R is selected from —H and —(C₁-C₆)alkyl;
[symbol] represents a carbon-carbon single bond or double bond, with the proviso that when X is a covalent bond [symbol] represents a carbon-carbon single bond;
spacer in each case independently represents a moiety comprising a linear chain of 6 to 20 atoms (e.g., selected from C, N, O, S and P) which attaches the ligand to the rest of the molecule;
ligand in each case is independently a monosaccharide (e.g., GalNAc);
linker and tether taken together represent a moiety which attaches the cargo to the rest of the molecule, e.g. comprising a linear chain of 8 to 30 atoms; and
cargo is a nucleic acid.

In embodiments, X is —C(O)—. In other embodiments, X is a covalent bond.

In embodiments, Y is —NR—, e.g., wherein R is selected from —H and —(C₁-C₃)alkyl. In embodiments, R is —H. In embodiments, R is —CH₃. In embodiments, Y is —NH—. In embodiments, Y is —N(CH₃)—.

In embodiments, X is —C(O)— and Y is —NR—, e.g., wherein R is selected from —H and —(C₁-C₃)alkyl. In embodiments, X is —C(O)— and Y is —NH—. In embodiments, X is —C(O)— and Y is —N(CH₃)—. In other embodiments, X is a covalent bond and Y is —NR—, e.g., wherein R is selected from —H and —(C₁-C₃)alkyl.

In embodiments, [symbol] represents a single bond. In other embodiments, [symbol] represents a double bond.

In embodiments, the compound is a compound having the structure of Formula (Ha):

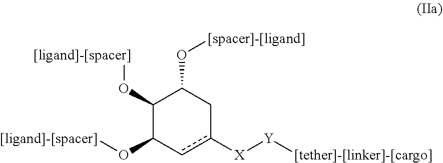

or a pharmaceutically acceptable salt thereof, wherein X, Y, ligand, spacer, tether, linker and cargo are as defined herein.

In embodiments, the compound is a compound having the structure of Formula (IIb):

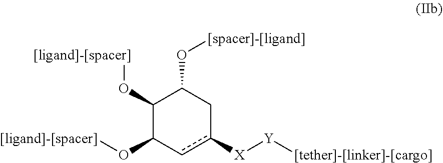

or a pharmaceutically acceptable salt thereof, wherein X, Y, ligand, spacer, tether, linker and cargo are as defined herein, and [symbol] represents a single bond.

In other embodiments, the compound is a compound having the structure of Formula (IIc):

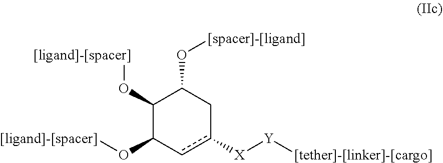

or a pharmaceutically acceptable salt thereof, wherein X, Y, ligand, spacer, tether, linker and cargo are as defined herein, and [symbol] represents a single bond.

In other embodiments, the compound is a compound having the structure of Formula (IId):

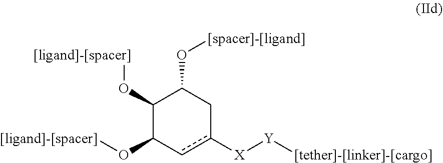

or a pharmaceutically acceptable salt thereof, wherein X, Y, ligand, spacer, tether, linker and cargo are as defined herein, and [symbol] represents a double bond.

In particular embodiments, the ligand in each case is N-acetylglucosamine (“GalNAc”) which may facilitate or promote binding of the compound to ASGPRs. This may target the delivery of cargo molecules to cells and tissues which express such receptors. Viewed from this aspect, the disclosure provides a compound, or a pharmaceutically acceptable salt thereof, the compound having the structure of Formula (III):

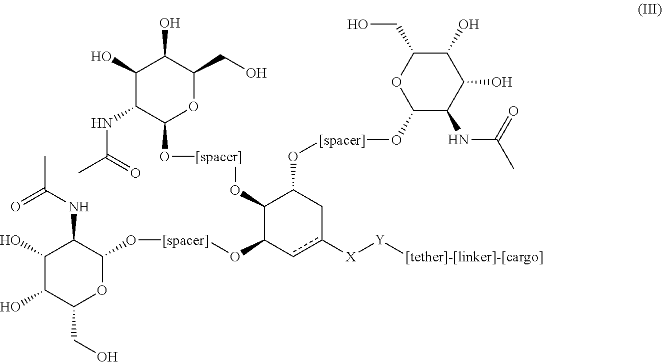

wherein X, Y, spacer, tether, linker and cargo are as defined herein, and [symbol] represents a carbon-carbon single bond or double bond, with the proviso that when X is a covalent bond [symbol] represents a carbon-carbon single bond.

In embodiments: X is —C(O)—; Y is —O— or —NR—, wherein R is selected from —H and —(C₁₋₆)alkyl; [symbol] represents a single bond; spacer in each case independently represents a moiety comprising a linear chain of 6 to 20 atoms (e.g., selected from C, N, O, S and P) which attaches the ligand to the rest of the molecule; linker and tether taken together represent a moiety which attaches the cargo to the rest of the molecule, e.g. comprising a linear chain of 8 to 30 atoms; and cargo is a nucleic acid.

In embodiments, the compound is a compound having the structure of Formula (IIIa):

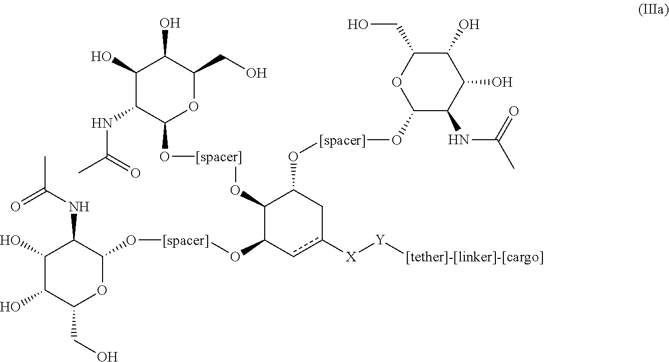

wherein X, Y, spacer, tether, linker and cargo are as defined herein. In embodiments, -represents a single bond.

In embodiments, the compound is a compound having the structure of Formula (IIIb):

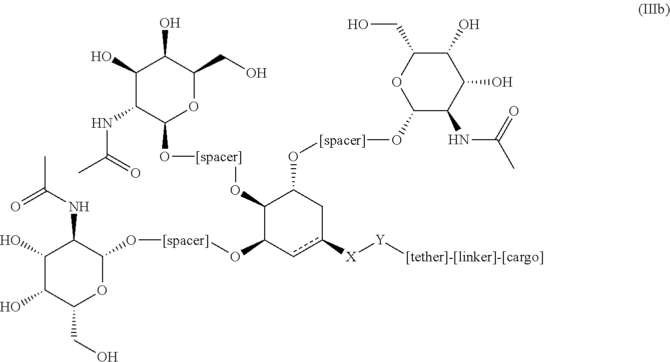

wherein X, Y, spacer, tether, linker and cargo are as defined herein, and [symbol] represents a single bond.

In other embodiments, the compound is a compound having the structure of Formula (IIIc):

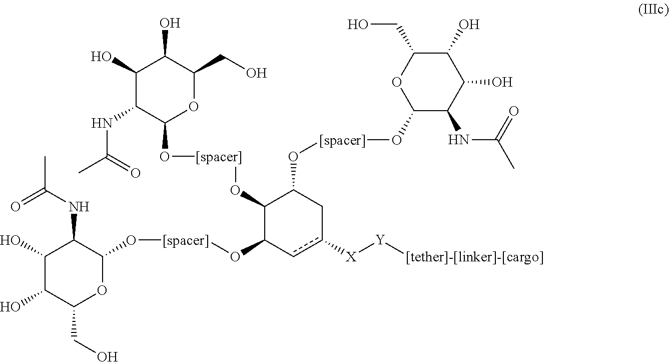

wherein X, Y, spacer, tether, linker and cargo are as defined herein, and [symbol] represents a single bond.

In other embodiments, the compound is a compound having the structure of Formula (IIId):

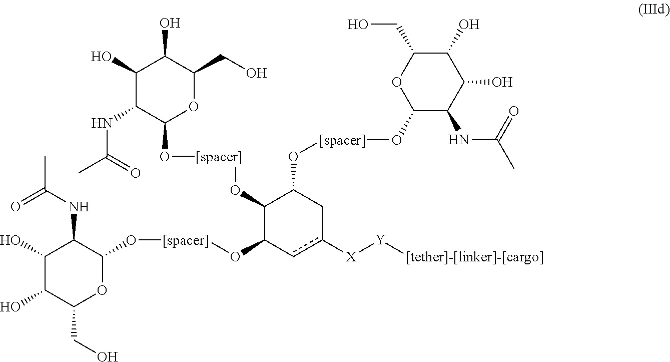

wherein X, Y, spacer, tether, linker and cargo are as defined herein, and [symbol] represents a double bond.

In particular embodiments, the cargo-containing arm is attached to the splitter via an amide, and the spacer includes an amide. Viewed from this aspect, the disclosure provides a compound, or a pharmaceutically acceptable salt thereof, the compound having the structure of Formula (IV):

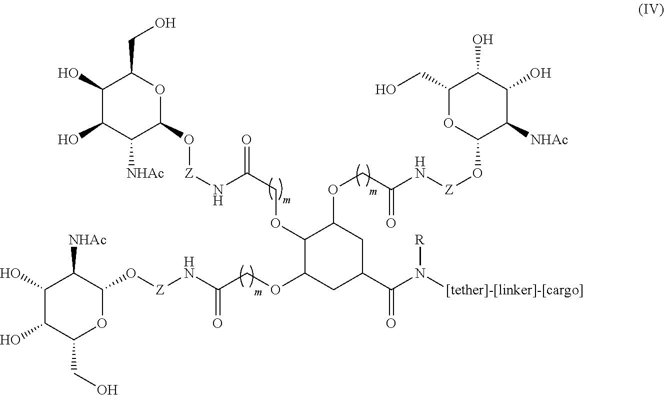

wherein:
Z in each case independently represents a moiety comprising a linear chain of 1 to 16 atoms (e.g., selected from C, N, O, S and P) which attaches the GalNAc ligand to the rest of the molecule;
m in each case is independently selected from the integers from 1 to 6;
R is selected from —H and —(C₁-C₆)alkyl; and
tether, linker and cargo are as defined herein.

In embodiments, Z in each case is independently selected from *—(C₁-C₁₆)alkylene-, *—(C₂-C₁₆)alkenylene-, *—(C₁-C₁₂)alkylene-C(O)—, *—(C₂-C₁₂)alkenylene-C(O)—, *—(C₁-C₅)alkylene-C(O)NR′—(C₁-C₆)alkylene-, *—(C₂-C₈)alkenylene-C(O)NR′—(C₁-C₆)alkylene-, *—(C₁-C₈)alkylene-C(O)NR′—(C₂-C₆)alkenylene- and —(C₂-C₈)alkenylene-C(O)NR′—(C₂-C₆)alkenylene-(where * in each case denotes the point of attachment to the GalNac oxygen atom), wherein R′ in each case is independently selected from —H and —(C₁-C₆)alkyl. In embodiments, Z in each case is independently selected from *—(C₁-C₁₆)alkylene-, *—(C₂-C₁₆)alkenylene-, *—(C₁-C₁₂)alkylene-C(O)—, *—(C₂-C₁₂)alkenylene-C(O)—, *—(C₁-C₅)alkylene-C(O)NR′—(C₁-C₆)alkylene-, *—(C₂-C₈)alkenylene-C(O)NR′—(C₁-C₆)alkylene-, *—(C₁-C₈)alkylene-C(O)NR′—(C₂-C₆)alkenylene- and *—(C₂-C₈)alkenylene-C(O)NR′—(C₂-C₆)alkenylene- (where * in each case denotes the point of attachment to the GalNac oxygen atom), wherein R′ in each case is independently selected from —H and —(C₁-C₆)alkyl. In embodiments, Z in each case is independently selected from *—(C₁-C₁₂)alkylene- and *—(C₁-C₆)alkylene-C(O)NR′—(C₁-C₄)alkylene-, wherein R′ in each case is independently selected from —H and —(C₁-C₆)alkyl. In embodiments, each Z is independently selected from *—(C₁-C₁₂)alkylene-, such as *—(C₁-C₁₀)alkylene-, *—(C₂-C₈)alkylene- or *—(C₄-C₇)alkylene-, e.g. wherein Z is n-hexylene. In other embodiments, each Z is independently selected from *—(C₁-C₆)alkylene-C(O)NR′—(C₁-C₄)alkylene- (wherein R′ in each case is independently selected from —H and —(C₁-C₆)alkyl), such as *—(C₂-C₅)alkylene-C(O)NR′—(C₂-C₄)alkylene-, e.g. wherein Z is*—(C₄)alkylene-C(O)NH—(C₃)alkylene-. In embodiments, each Z is the same.

In embodiments, m in each case is independently selected from the integers from 1 to 5. In embodiments, m in each case is independently selected from 1, 2, 3 and 4. In embodiments, m in each case is independently selected from 1, 2, and 3. In embodiments, each m is the same. In embodiments, m is 1. In other embodiments, m is 2. In other embodiments, m is 3.

In embodiments, R is —H. In embodiments, R is —CH₃.

In embodiments, the compound is a compound having the structure of Formula (IVa):

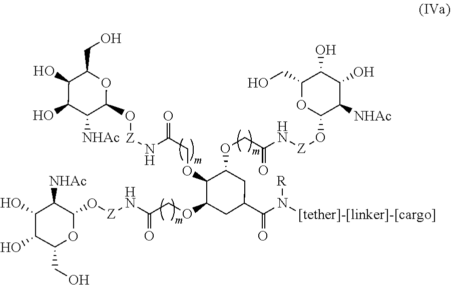

wherein Z, m, R, tether, linker and cargo are as defined herein.

In embodiments, the compound is a compound having the structure of Formula (IVb):

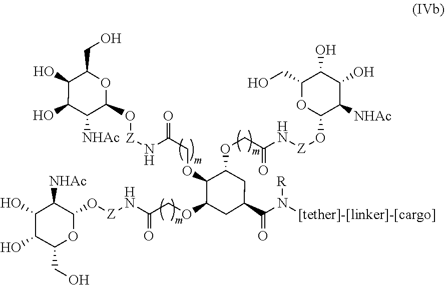

wherein Z, m, R, tether, linker and cargo are as defined herein.

In other embodiments, the compound is a compound having the structure of Formula (IVc):

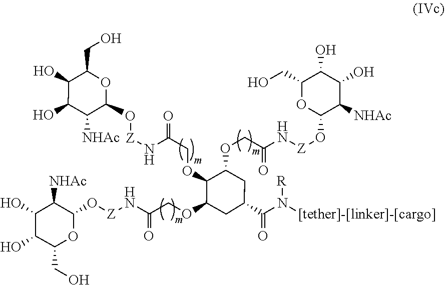

wherein Z, m, R, tether, linker and cargo are as defined herein. In another aspect, the cargo-containing arm is attached to the splitter via an amide, and the spacer includes a reverse amide. Viewed from this aspect, the disclosure provides a compound, or a pharmaceutically acceptable salt thereof, the compound having the structure of Formula (V):

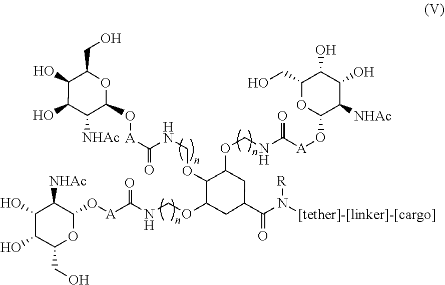

wherein:
A in each case independently represents a moiety comprising a linear chain of 1 to 16 atoms (e.g., selected from C, N, O, S and P) which attaches the GalNAc ligand to the rest of the molecule;
n in each case is independently selected from the integers from 1 to 6;
R is selected from —H and —(C₁-C₆)alkyl; and
tether, linker and cargo are as defined herein.

In embodiments, A in each case is independently selected from *—(C₁-C₁₆)alkylene-, and *—(C₂-C₁₆)alkenylene- (where * in each case denotes the point of attachment to the GalNac oxygen atom). In embodiments, A in each case is independently selected from *—(C₁-C₁₂)alkylene-, such as *—(C₂-C₁₀)alkylene- or *—(C₄-C₈)alkylene-, e.g. wherein A is n-pentylene. In embodiments, each A is the same.

In embodiments, n in each case is independently selected from the integers from 1 to 5. In embodiments, n in each case is independently selected from 1, 2, 3 and 4. In embodiments, n in each case is independently selected from 2, 3, and 4. In embodiments, each n is the same.

In embodiments, n is 3.

In embodiments, R is —H. In embodiments, R is —CH₃.

In embodiments, the compound is a compound having the structure of Formula (Va):

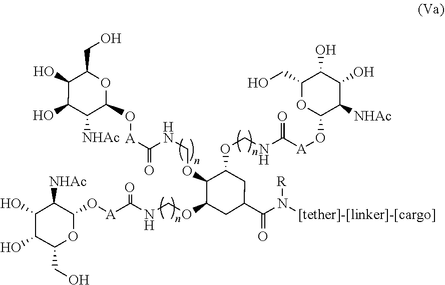

wherein A, n, R, tether, linker and cargo are as defined herein.

In embodiments, the compound is a compound having the structure of Formula (Vb):

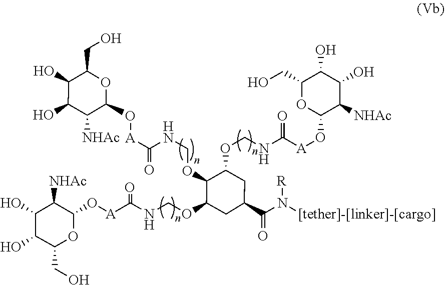

wherein A, n, R, tether, linker and cargo are as defined herein.

In other embodiments, the compound is a compound having the structure of Formula (Vc):

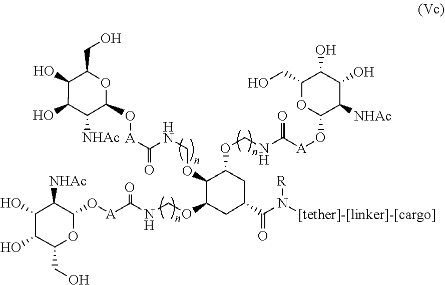

wherein A, n, R, tether, linker and cargo are as defined herein.

In particular embodiments, the shikimic acid core is not fully saturated. Viewed from this aspect, the disclosure provides a compound, or a pharmaceutically acceptable salt thereof, the compound having the structure of Formula (IX):

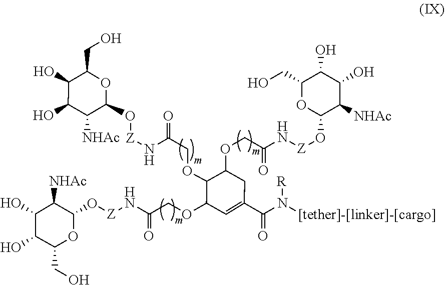

wherein:
Z in each case independently represents a moiety comprising a linear chain of 1 to 16 atoms (e.g., selected from C, N, O, S and P) which attaches the GalNAc ligand to the rest of the molecule;
m in each case is independently selected from the integers from 1 to 6;
R is selected from —H and —(C₁-C₆)alkyl; and
tether, linker and cargo are as defined herein.

In embodiments, each Z is independently selected from *—(C₁-C₁₂)alkylene-, such as *—(C₂-C₈)alkylene- or *—(C₄-C₇)alkylene-, e.g. wherein Z is n-hexylene (where * in each case denotes the point of attachment to the GalNAc oxygen atom). In embodiments, each Z is the same.

In embodiments, m in each case is independently selected from the integers 1, 2, and 3. In embodiments, each m is the same. In embodiments, m is 1. In other embodiments, m is 2. In other embodiments, m is 3.

In embodiments, R is —H. In embodiments, R is —CH₃.

In embodiments, the compound is a compound having the structure of Formula (IXa):

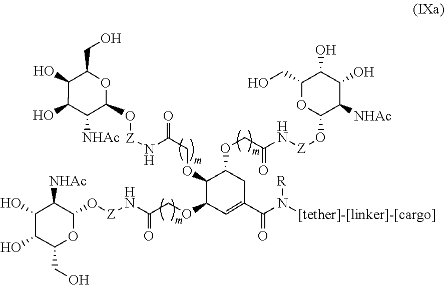

wherein Z, m, R, tether, linker and cargo are as defined herein.

In particular embodiments, the spacer and linker-tether components comprise alkylene chains and amide groups, e.g. as defined herein. Viewed from this aspect, the disclosure provides a compound, or a pharmaceutically acceptable salt thereof, the compound having the structure of Formula (XI):

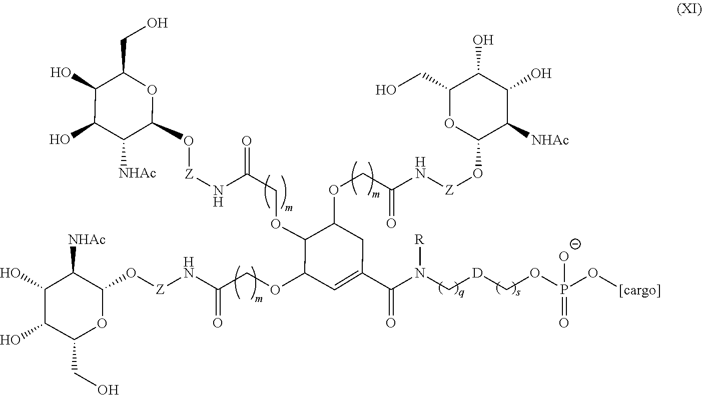

wherein Z, m, R, q, D, s and [cargo] are as defined herein.

In embodiments:
Z in each case is independently selected from *—(C₁-C₁₂)alkylene- and *—(C₁-C₆)alkylene-C(O)NR′—(C₁-C₄)alkylene-, wherein R′ in each case is independently selected from —H and —(C₁-C₆)alkyl (where * in each case denotes the point of attachment to the GalNac oxygen atom);
m in each case is independently selected from the integers from 1 to 6;
R is selected from —H and —(C₁-C₆)alkyl;
q is an integer from 1 to 12; D is selected from (i) —C(O)NH—, and (ii) —NHC(O)—(CH₂)—C(O)NH— (wherein t is an integer from 1 to 6);
s is an integer from 0 to 12; and cargo is as defined herein (e.g., an ASO or siRNA).

In embodiments, each Z is independently selected from *—(C₁-C₁₂)alkylene-. In embodiments, each Z is n-hexylene. In embodiments, each Z is n-hexylene and each m is 2. In other embodiments, each Z is independently selected from *—(C₁-C₆)alkylene-C(O)NR′—(C₁-C₄)alkylene- (wherein R′ in each case is independently selected from —H and —(C₁-C₆)alkyl). In embodiments, each Z is *—(CH₂)₄-C(O)NH—(CH₂)₃-.

In embodiments, each Z is *—(CH₂)₄-C(O)NH—(CH₂)₃— and each m is 2.

In embodiments, R is hydrogen.

In embodiments, D is —C(O)NH—, q is an integer from 1 to 8, and s is an integer from 1 to 8.

In embodiments, D is —C(O)NH—, q is 5, and s is 6. In other embodiments, D is —NHC(O)—(CH₂)ₜ-C(O)NH— (wherein t is an integer from 1 to 6), q is an integer from 1 to 8, and s is an integer from 1 to 8. In embodiments, D is —NHC(O)—(CH₂)ₜ-C(O)NH— (wherein t is 4), q is 4, and s is 6.

In embodiments, the compound is a compound having the structure of Formula (XIa):

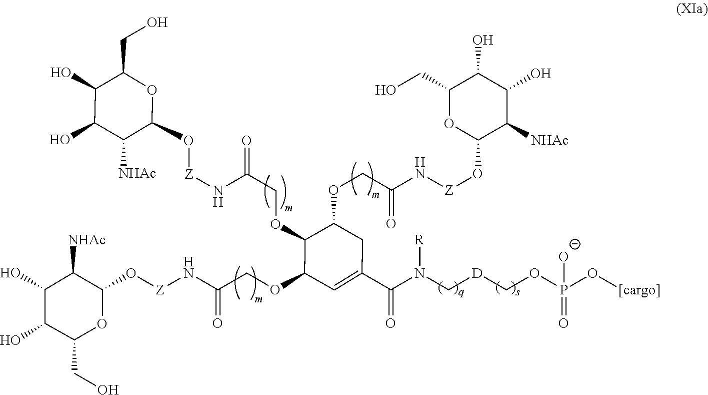

wherein Z, m, R, q, D, s and [cargo] are as defined herein.

In particular embodiments, the spacer and linker-tether components comprise specific alkylene chains and amide groups. Viewed from this aspect, the disclosure provides a compound, or a pharmaceutically acceptable salt thereof, the compound having the structure of Formula (XIII):

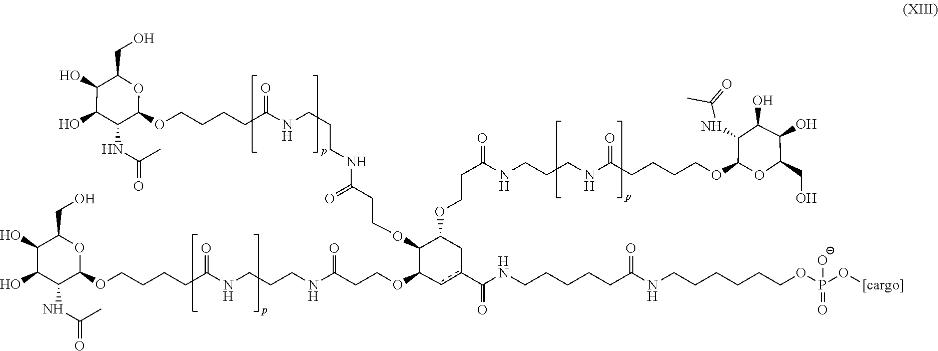

wherein: each p is independently selected from 0 and 1; [symbol] represents a carbon-carbon single bond or double bond; and cargo is as defined herein (e.g., an ASO or siRNA).

In embodiments, each p is 1. In embodiments, each p is 0. In embodiments, [symbol] represents a carbon-carbon single bond. In embodiments, [symbol] represents a carbon-carbon double bond. In embodiments, each p is 1 and [symbol] represents a carbon-carbon double bond. In other embodiments, each p is 1 and [symbol] represents a carbon-carbon single bond. In other embodiments, each p is 0 and [symbol] represents a carbon-carbon double bond. In other embodiments, each p is 0 and [symbol] represents a carbon-carbon single bond. In embodiments, the carbon atom on the shikimic acid core to which the carbonyl group is attached is in the (S) configuration.

In embodiments, the compound is a compound as illustrated in any one of the Examples described below, e.g. a compound obtainable by (or obtained by) a process as described in Example 1. Viewed from this aspect the disclosure provides a compound selected from:

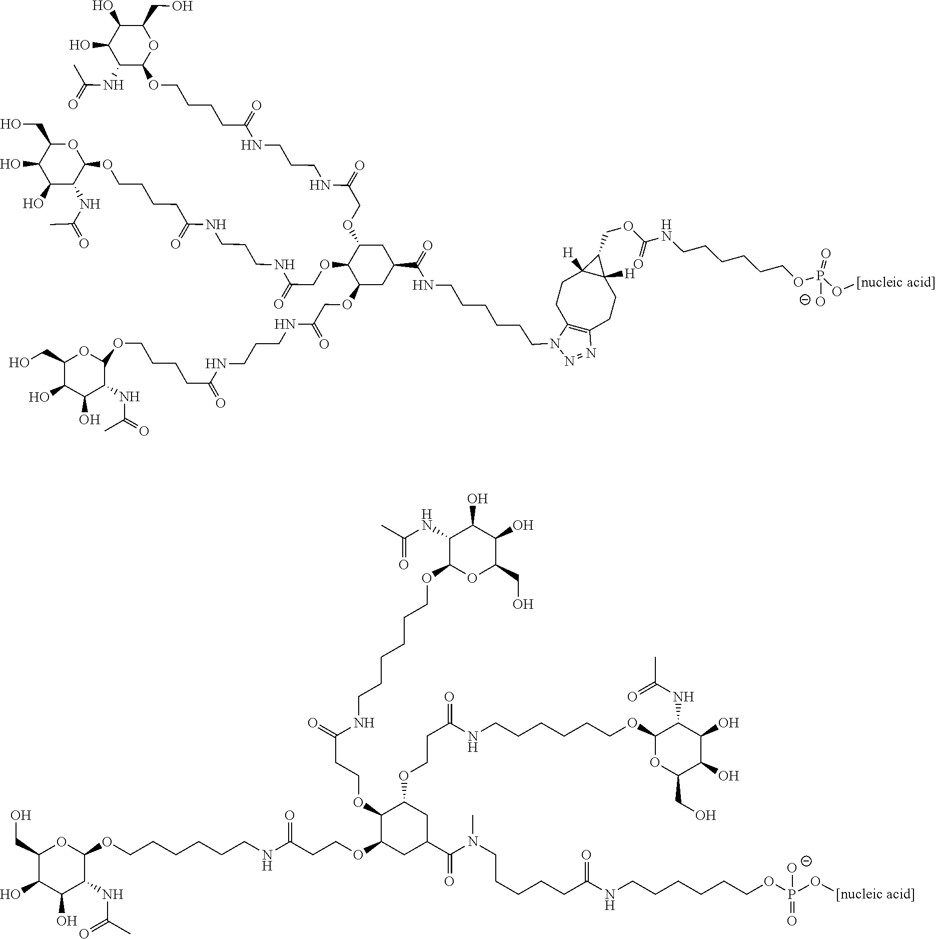

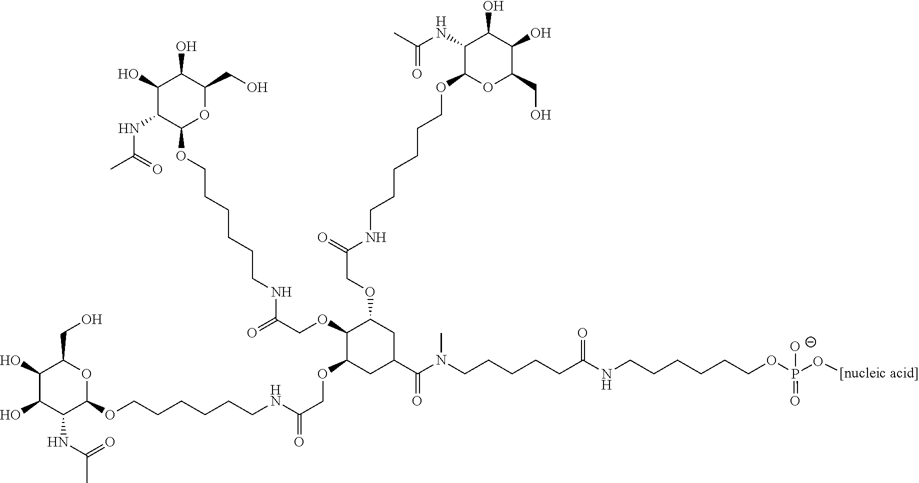

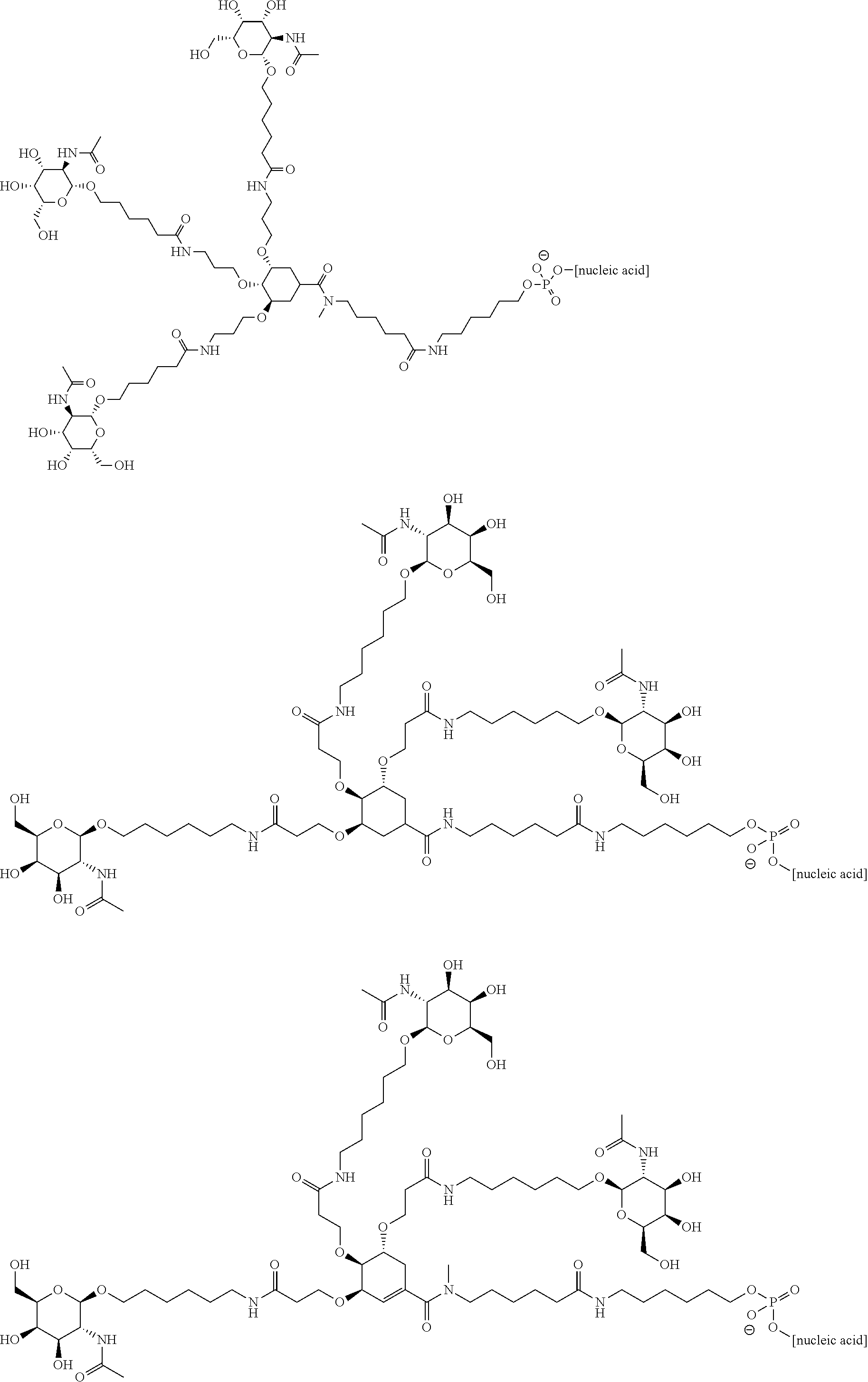

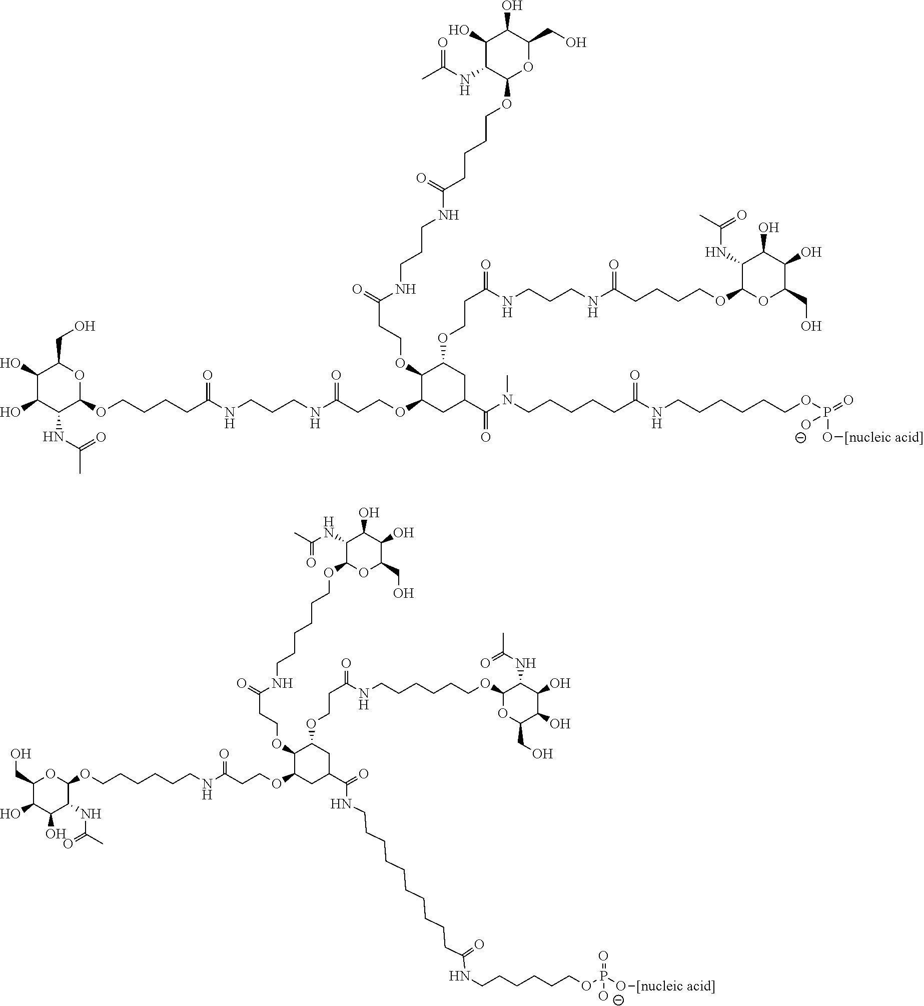

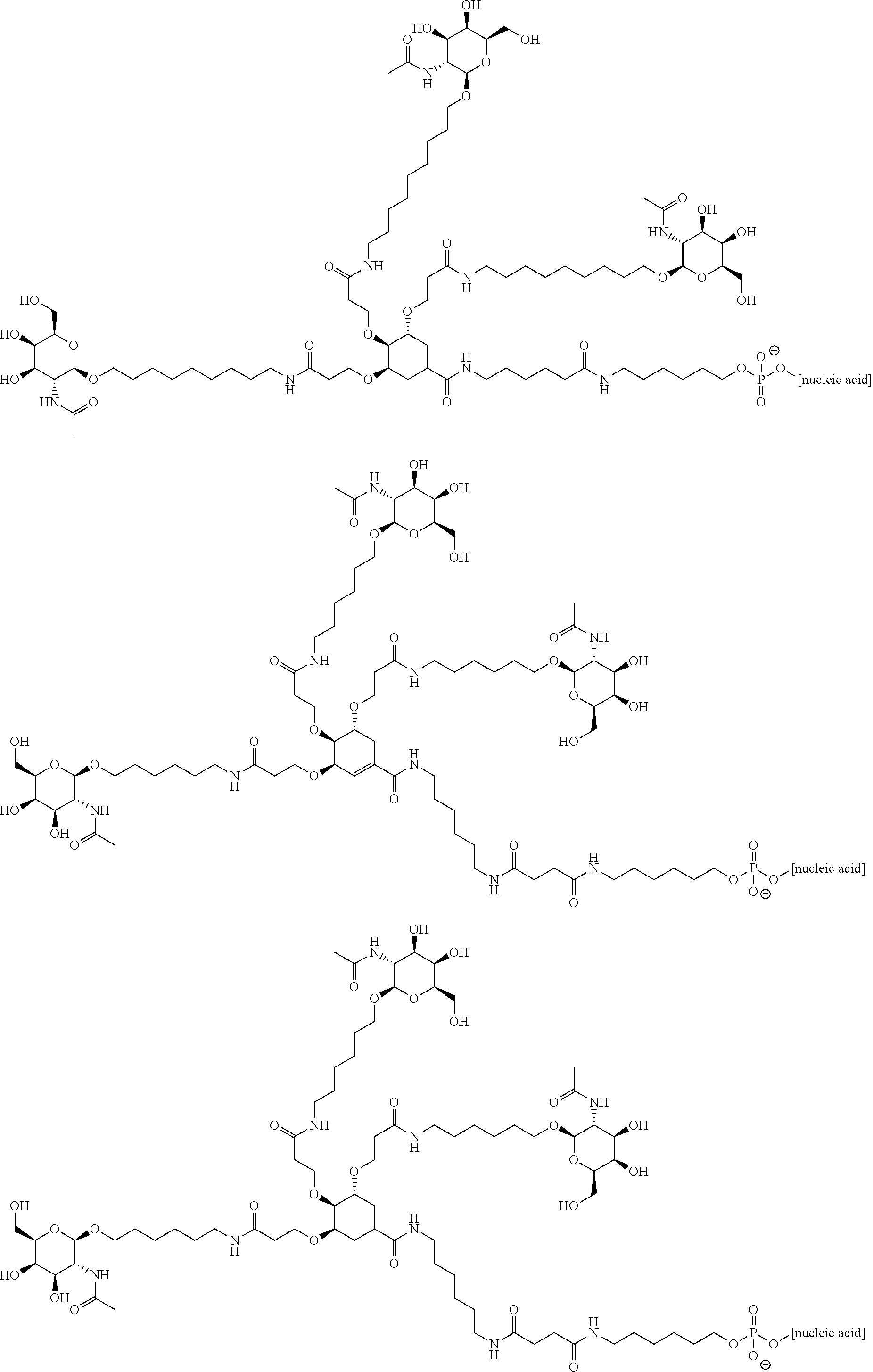

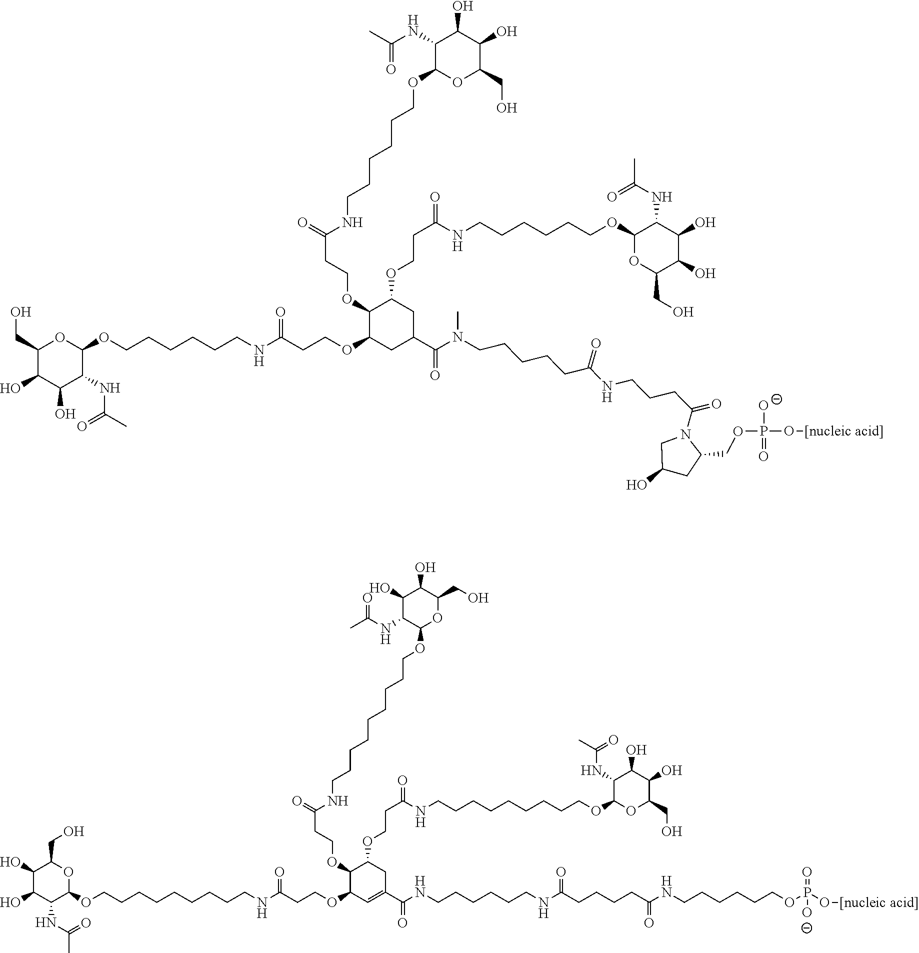

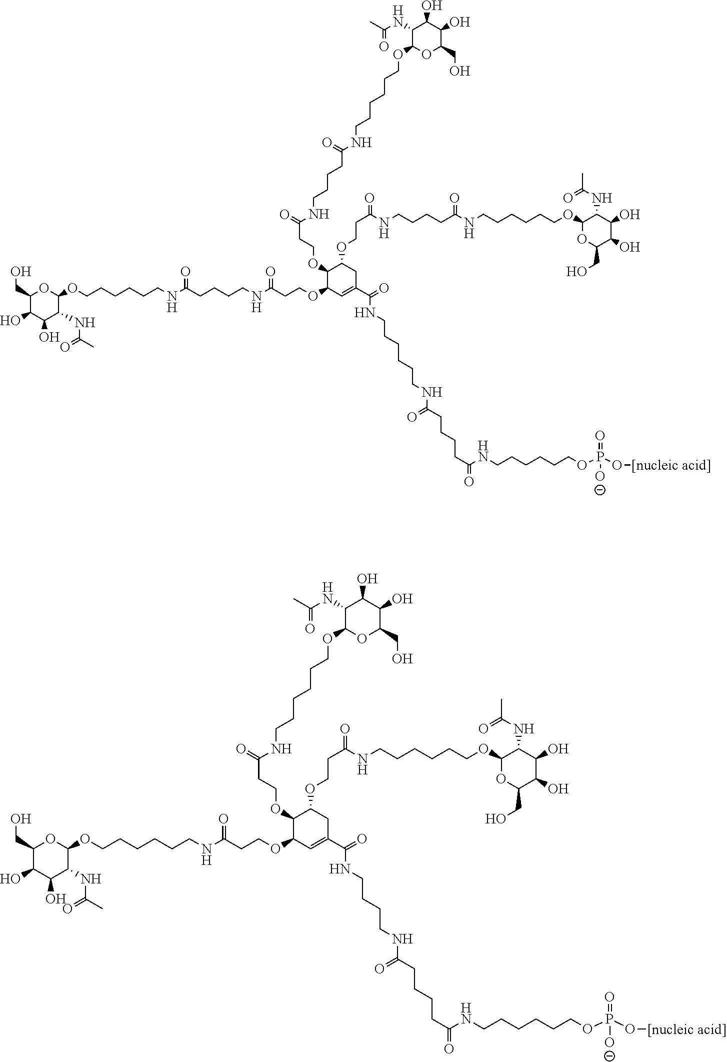

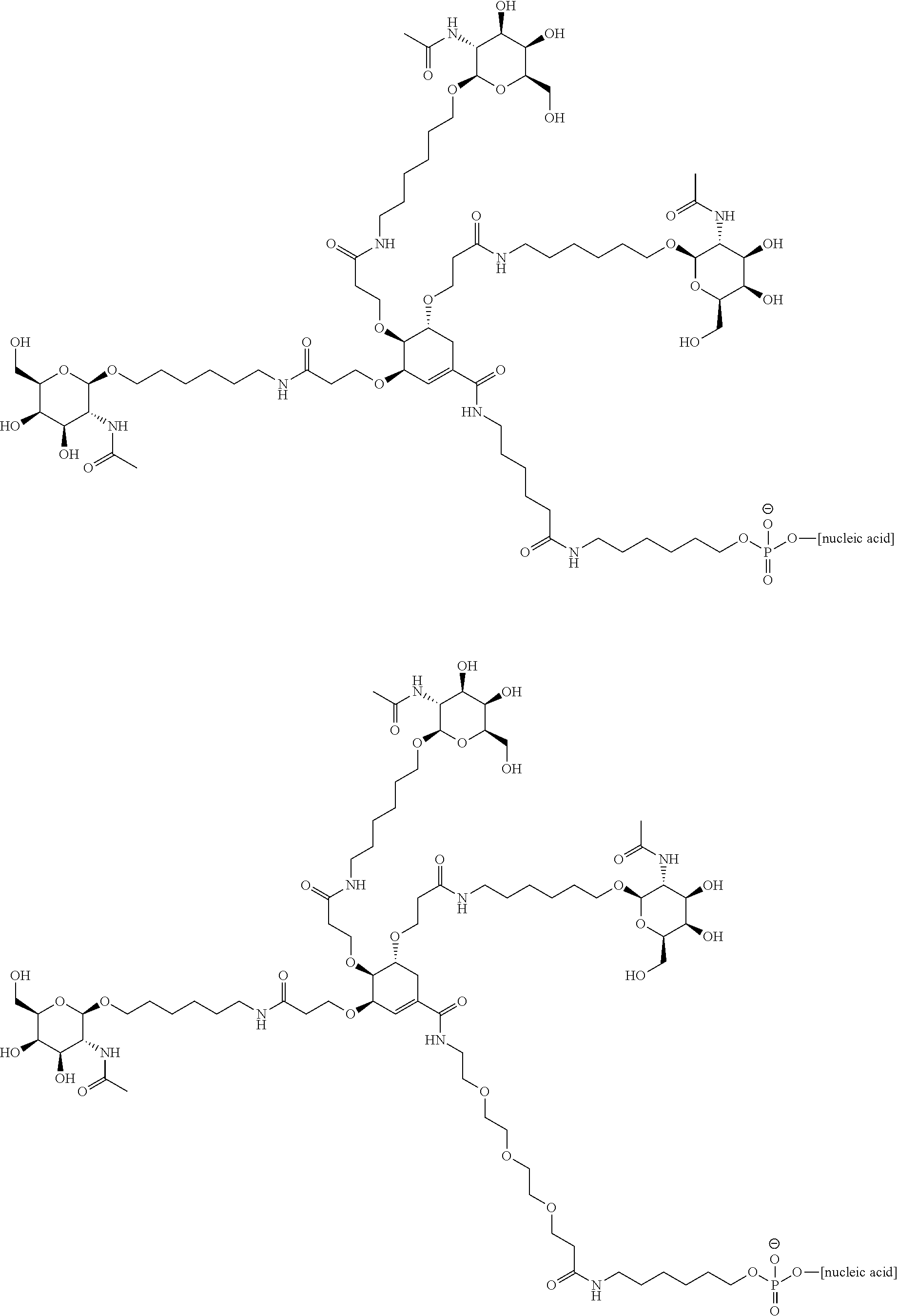

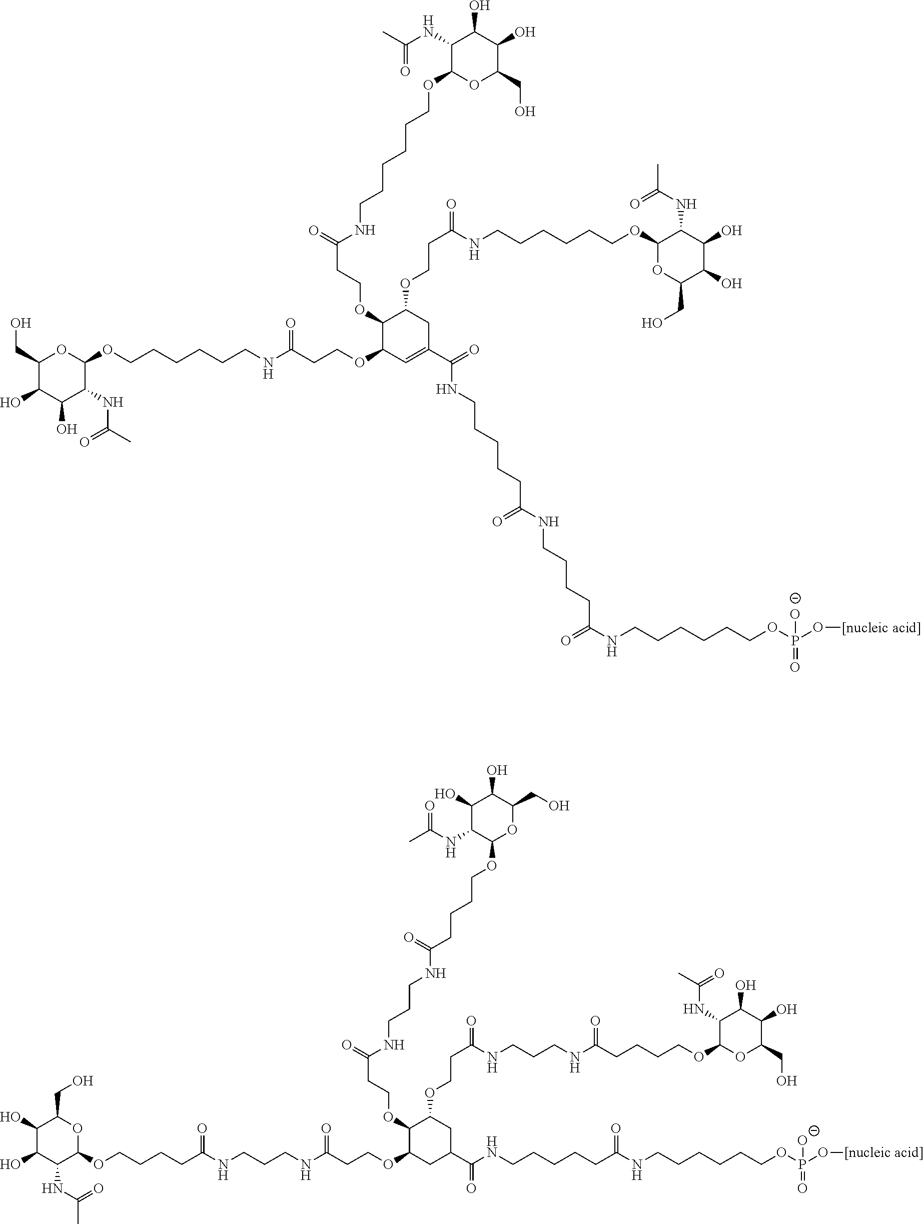

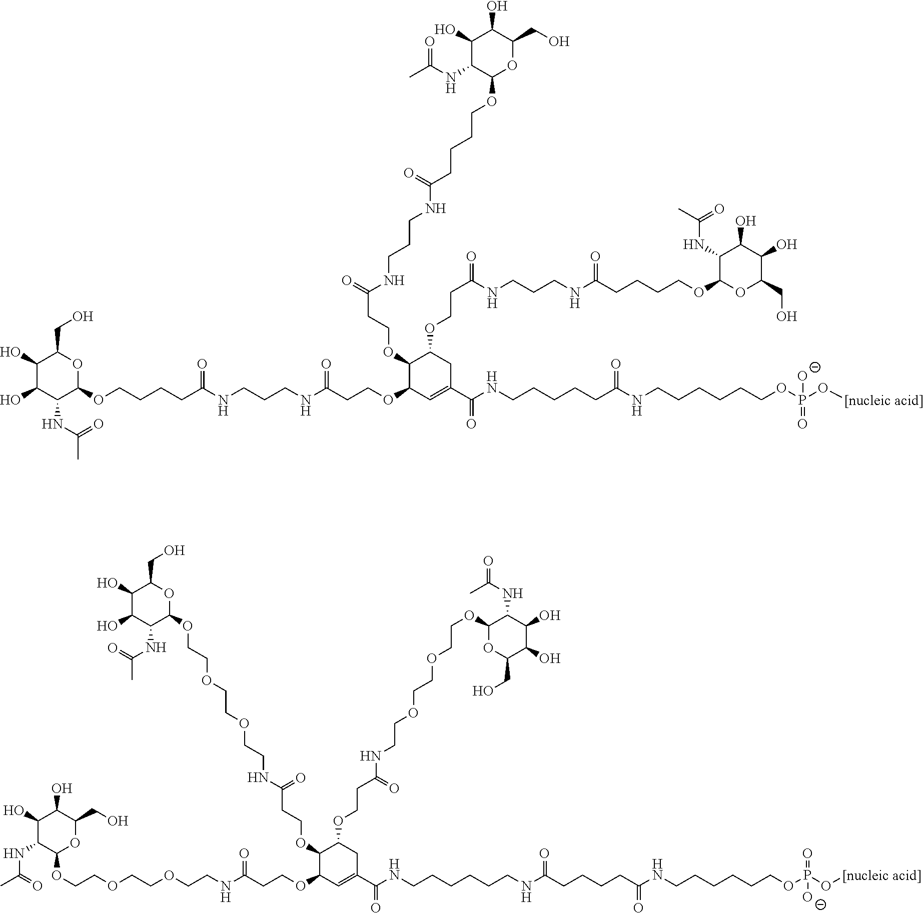

and the pharmaceutically acceptable salts thereof, wherein “nucleic acid” denotes a cargo nucleic acid as defined herein.

In embodiments, the compound is selected from:

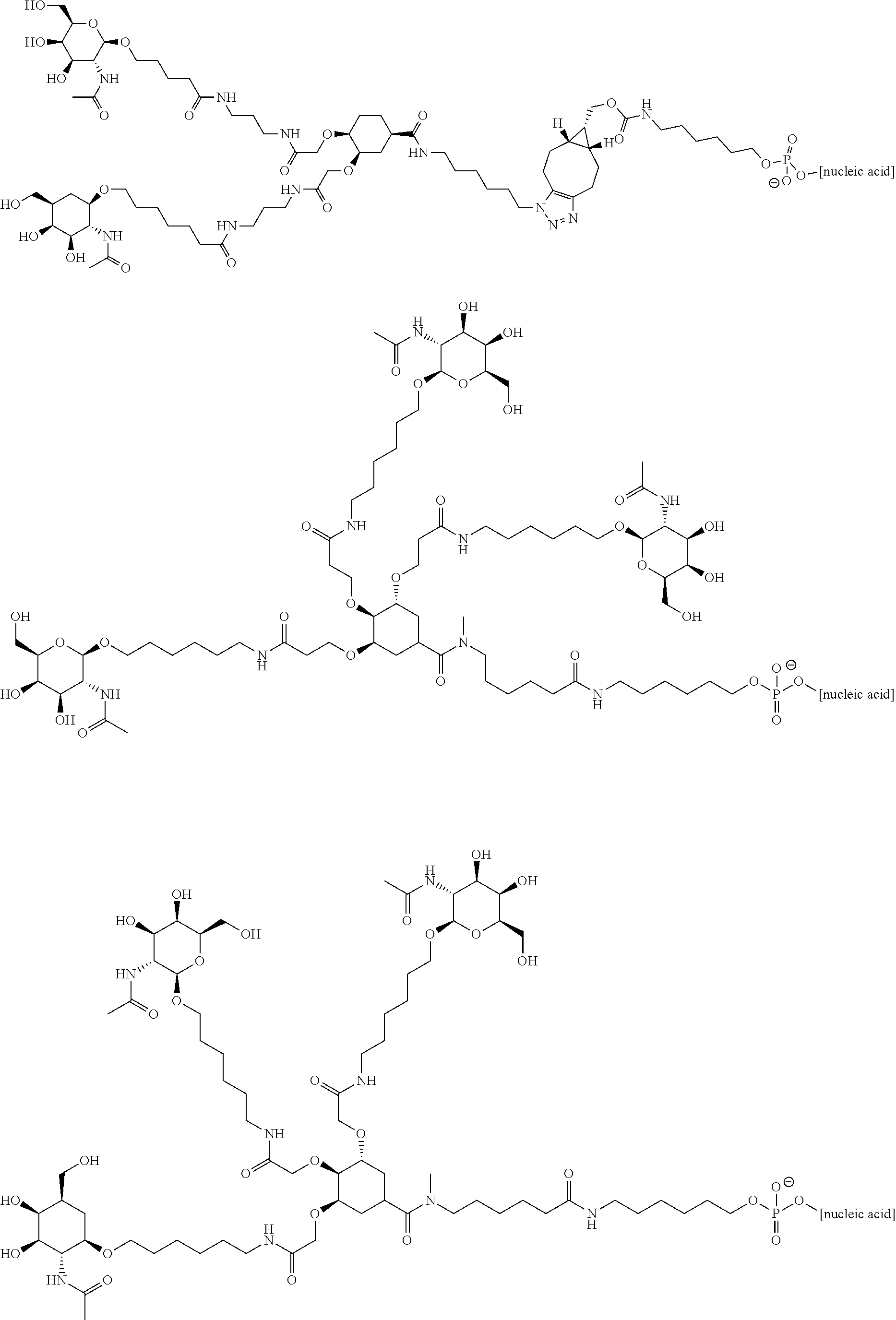

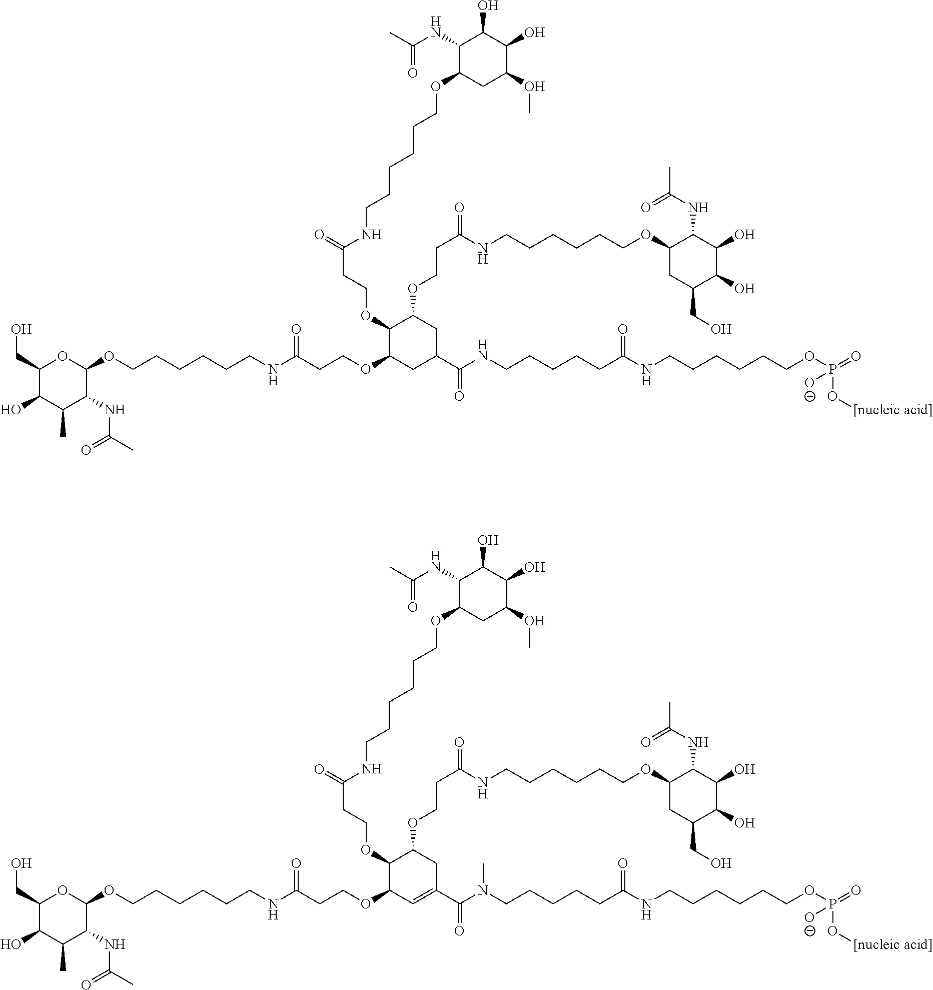

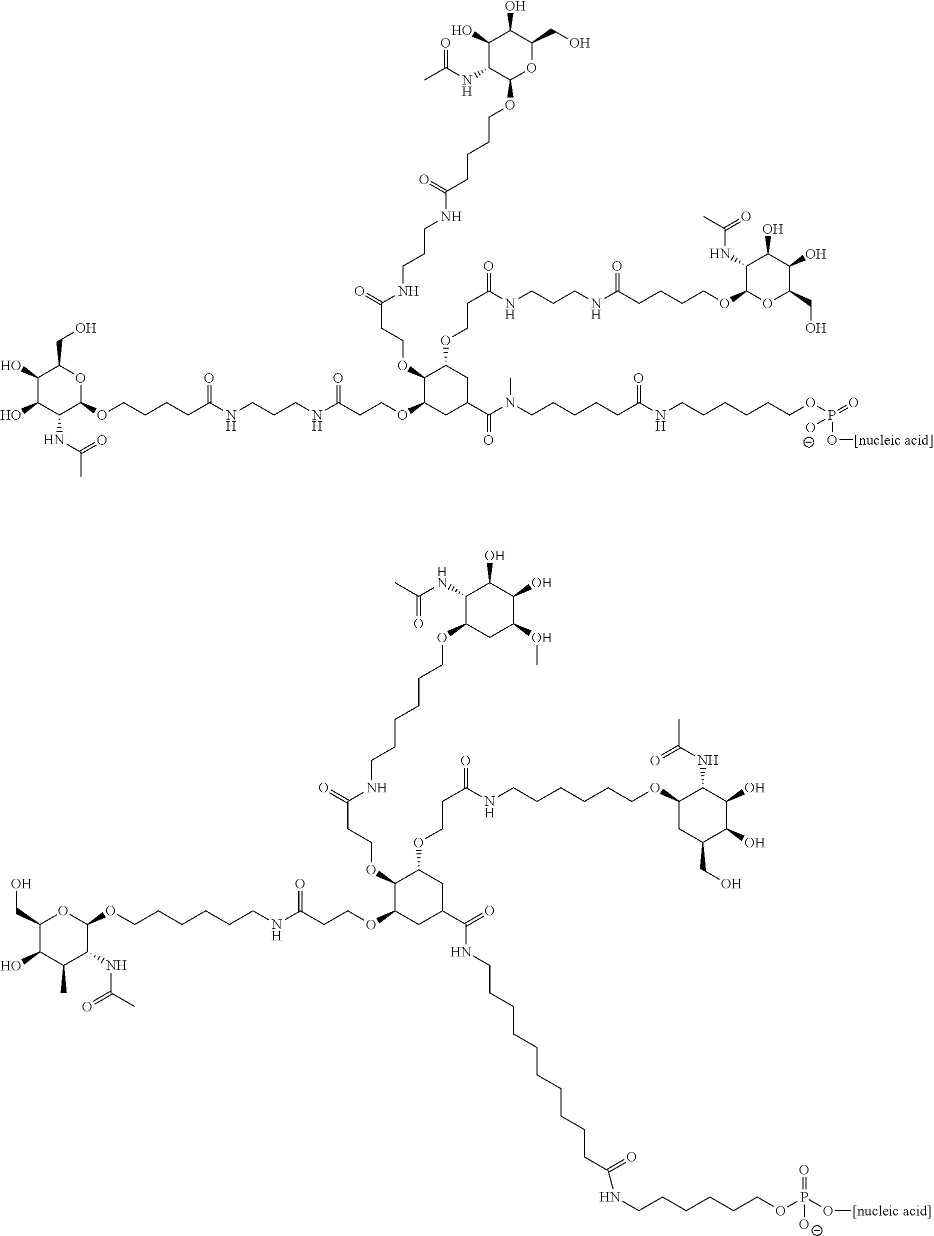

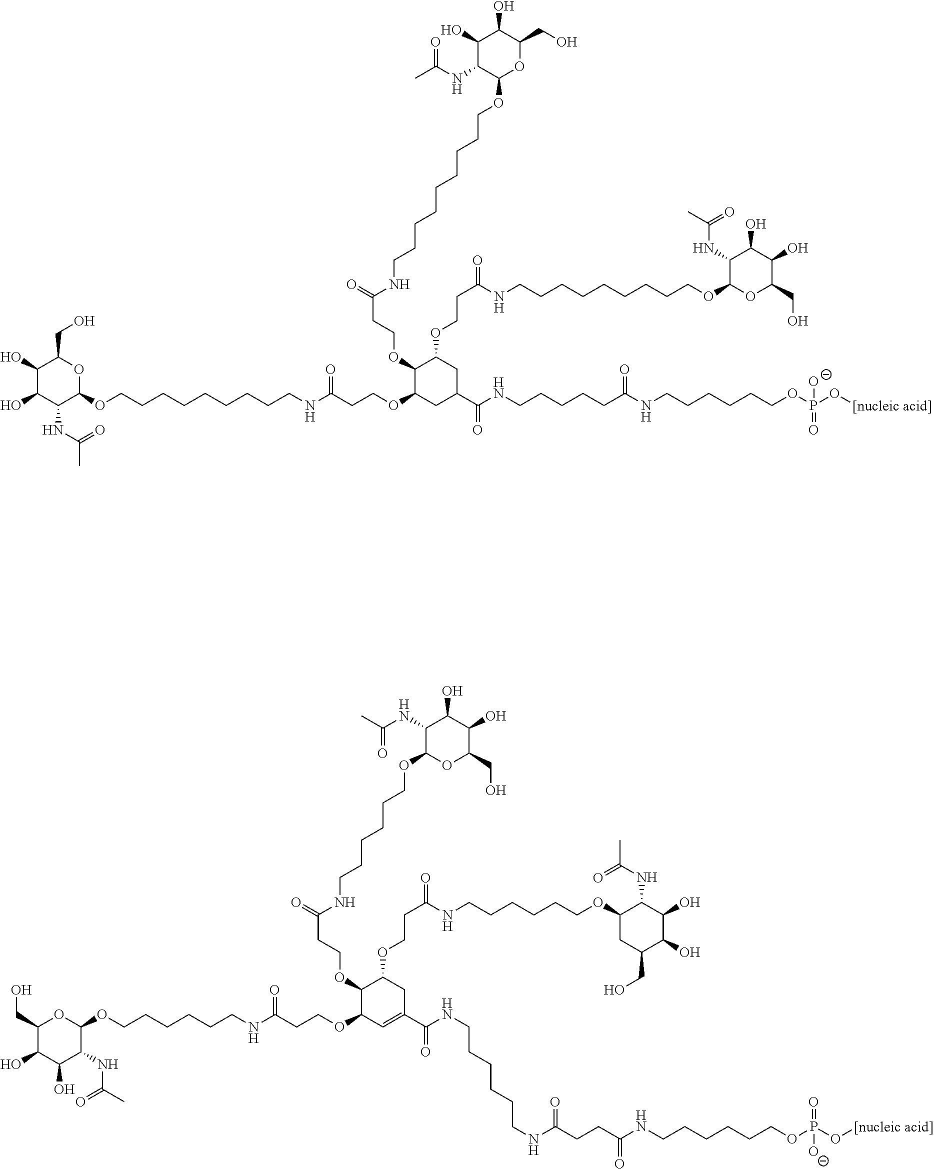

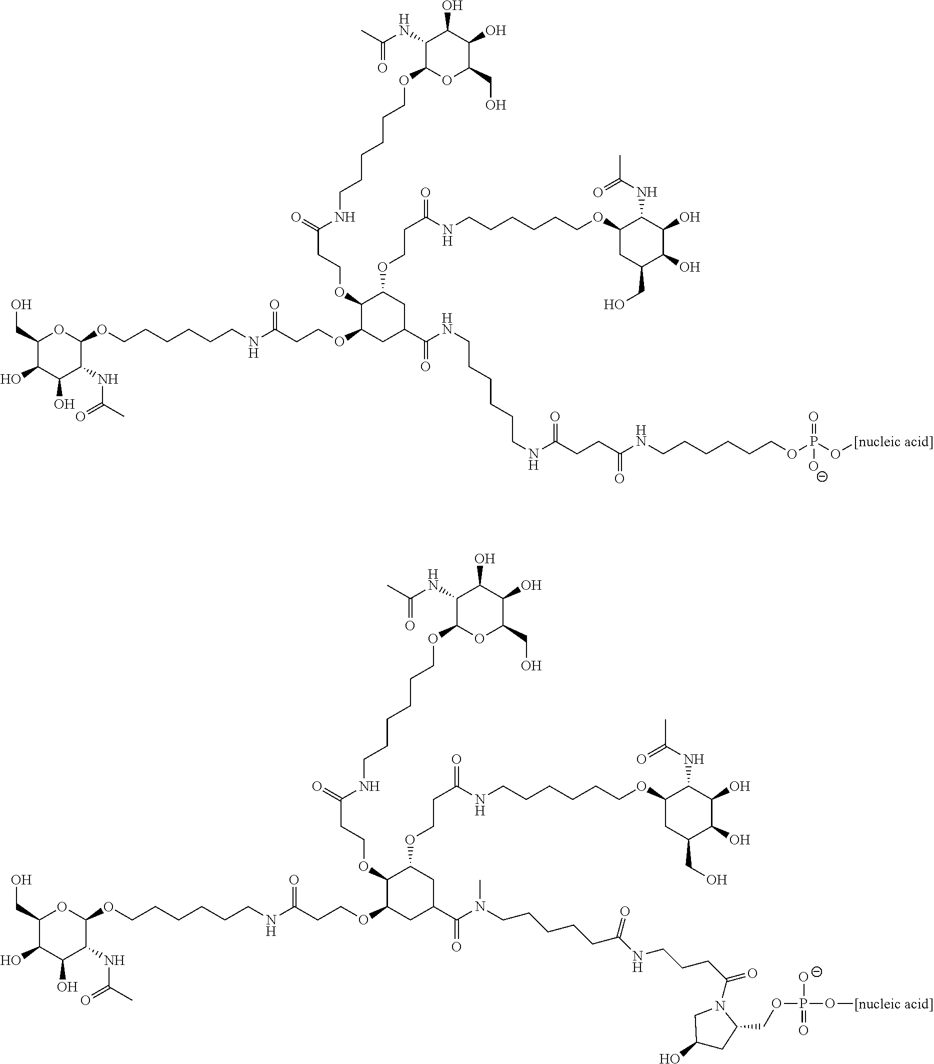

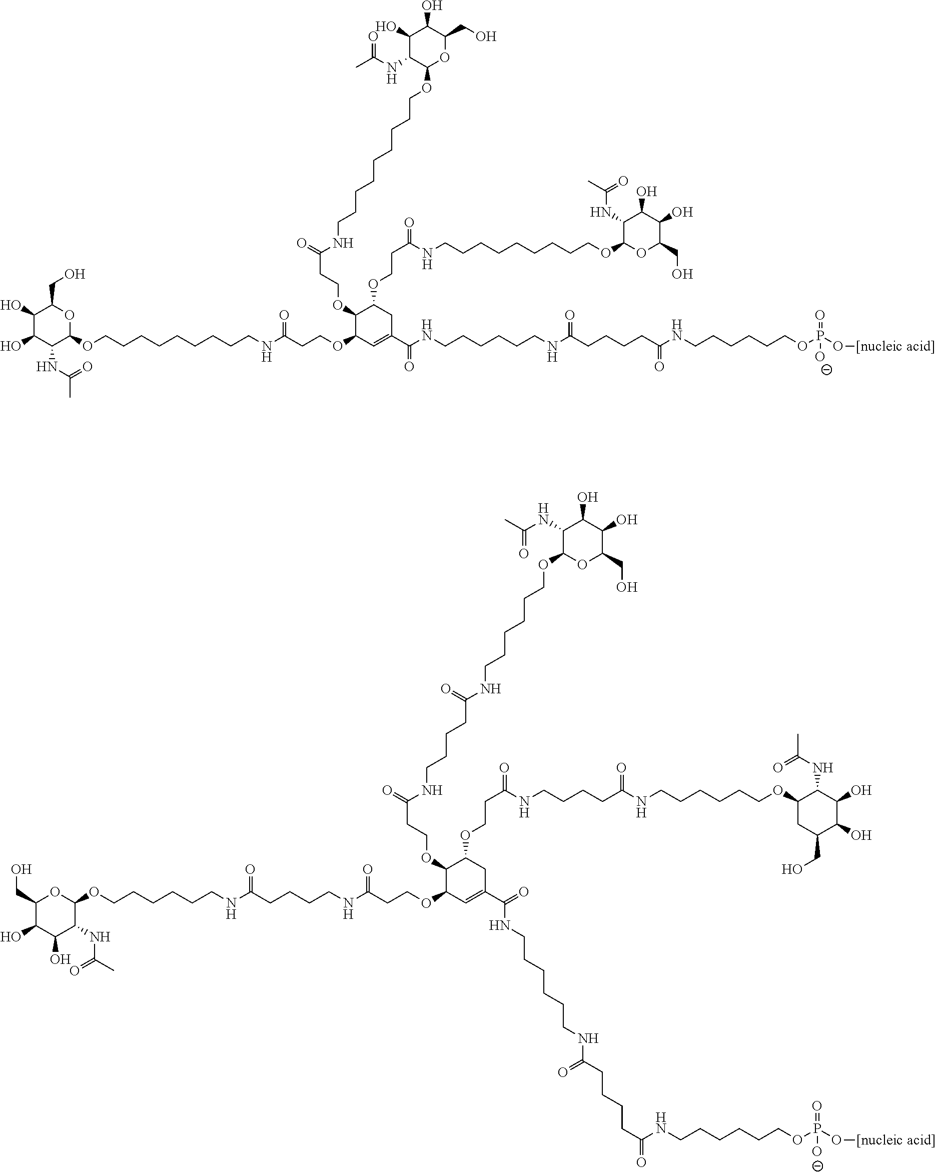

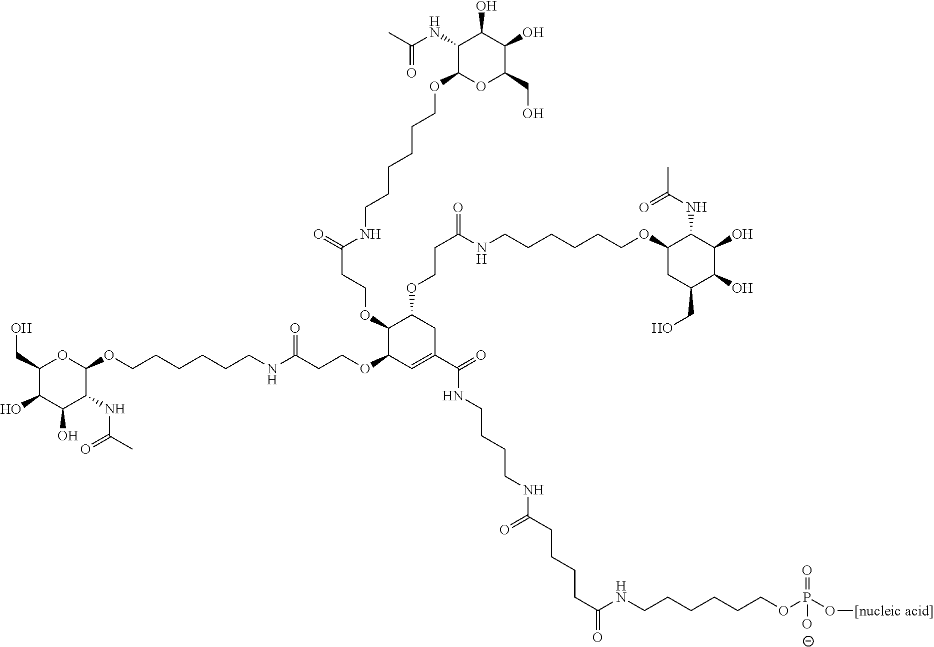

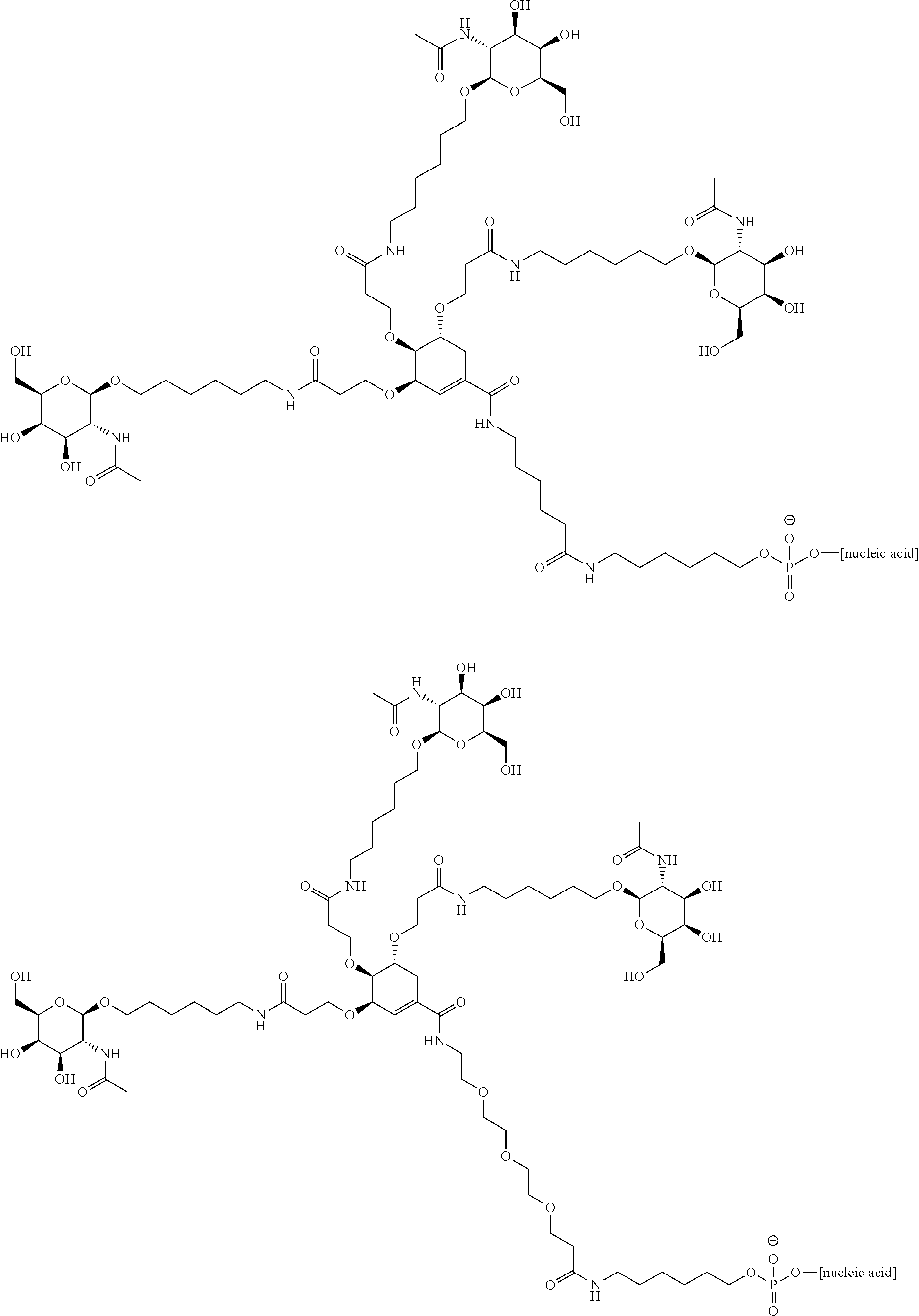

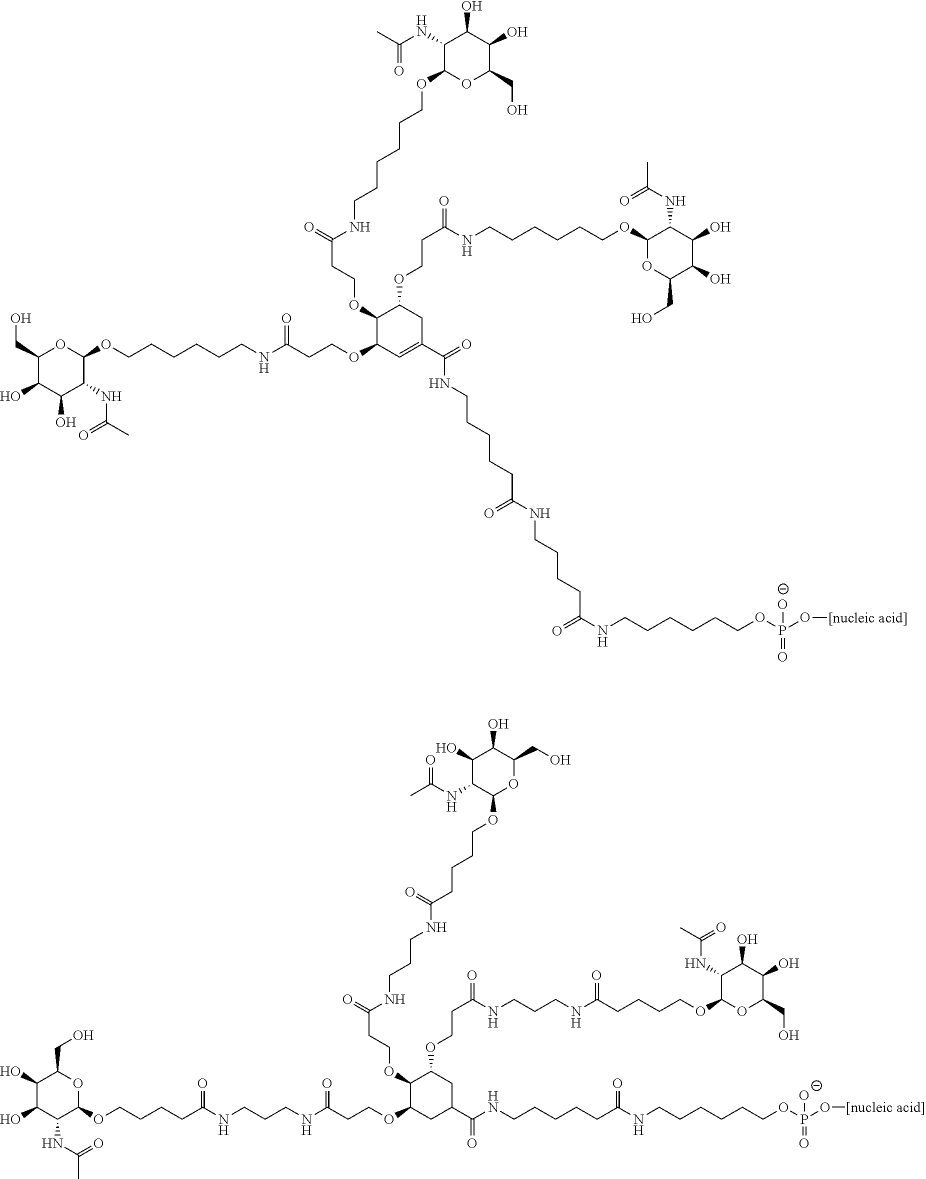

and the pharmaceutically acceptable salts thereof, wherein “nucleic acid” denotes a cargo nucleic acid as defined herein.

In embodiments, the compound is selected from:

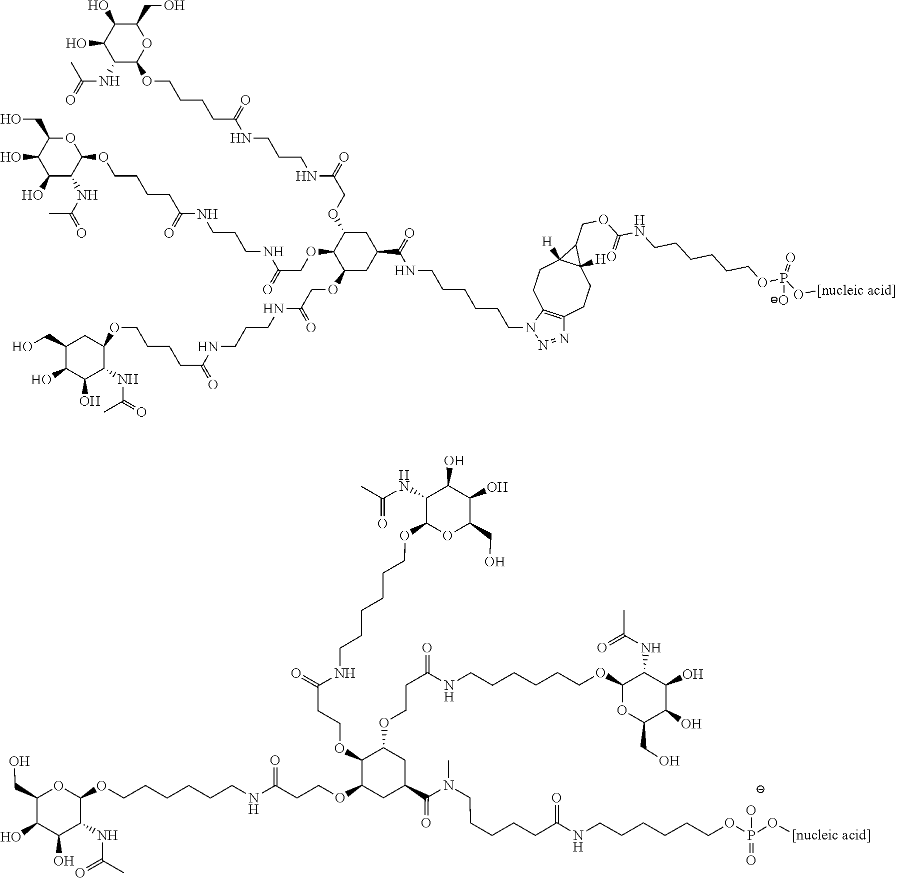

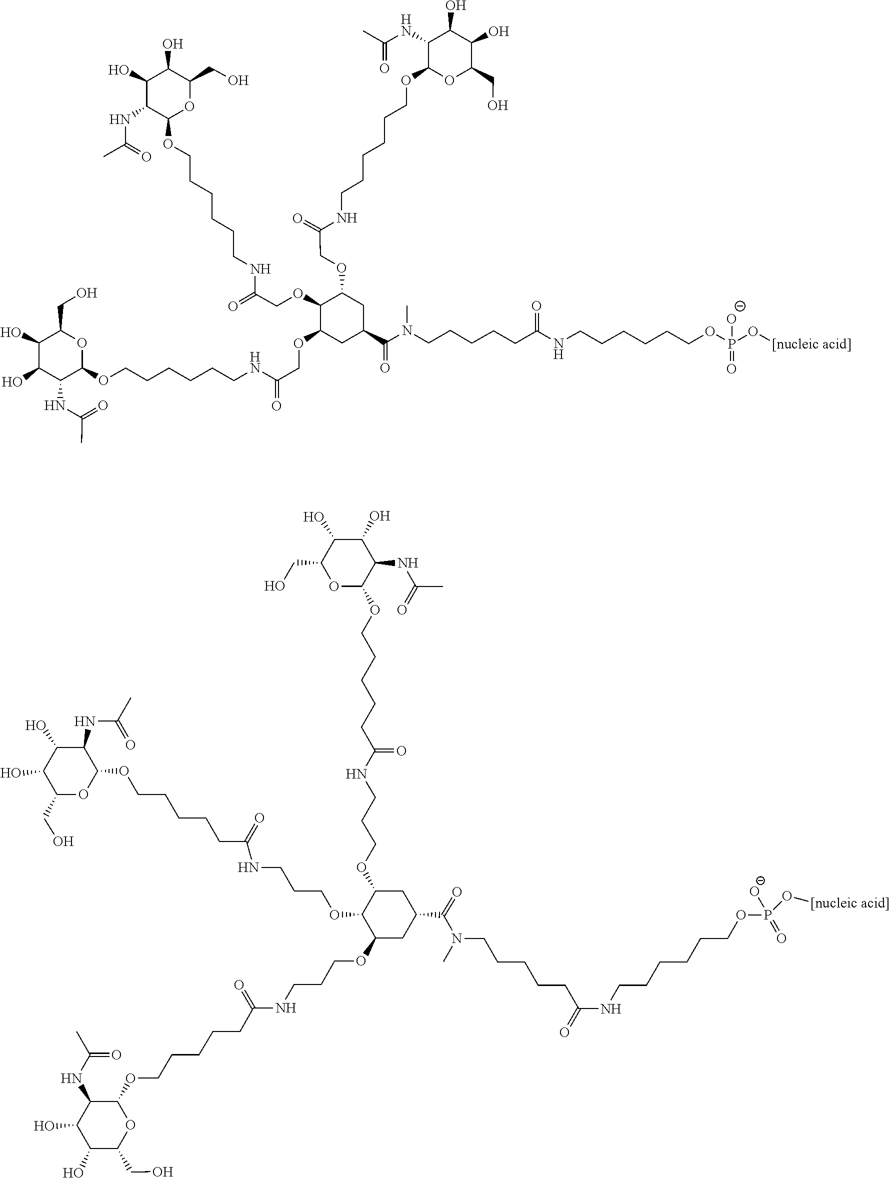

and the pharmaceutically acceptable salts thereof, wherein “nucleic acid” denotes a cargo nucleic acid as defined herein.

In embodiments, the nucleic acid is an ASO (e.g., a MALAT1 ASO having a sequence comprising SEQ ID NO: 1). In other embodiments, the nucleic acid is a siRNA (e.g., an ANGPTL3 siRNA having a sequence comprising SEQ ID NO: 2 and/or SEQ ID NO: 3). In embodiments, the siRNA is a PPTB siRNA (e.g., having a sequence comprising SEQ ID NO: 4, 5 and/or 6, for example comprising SEQ ID NOs: 4 and 6, or comprising SEQ ID NOs: 5 and 6).

In embodiments, the compound is selected from:

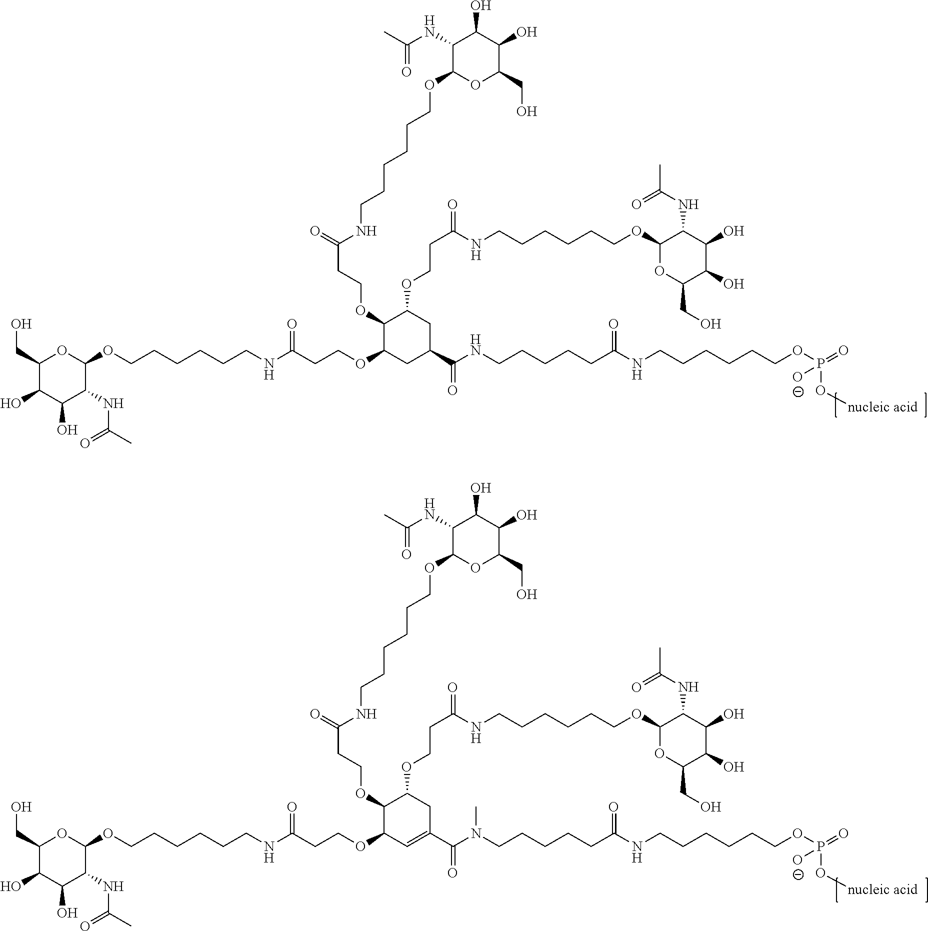

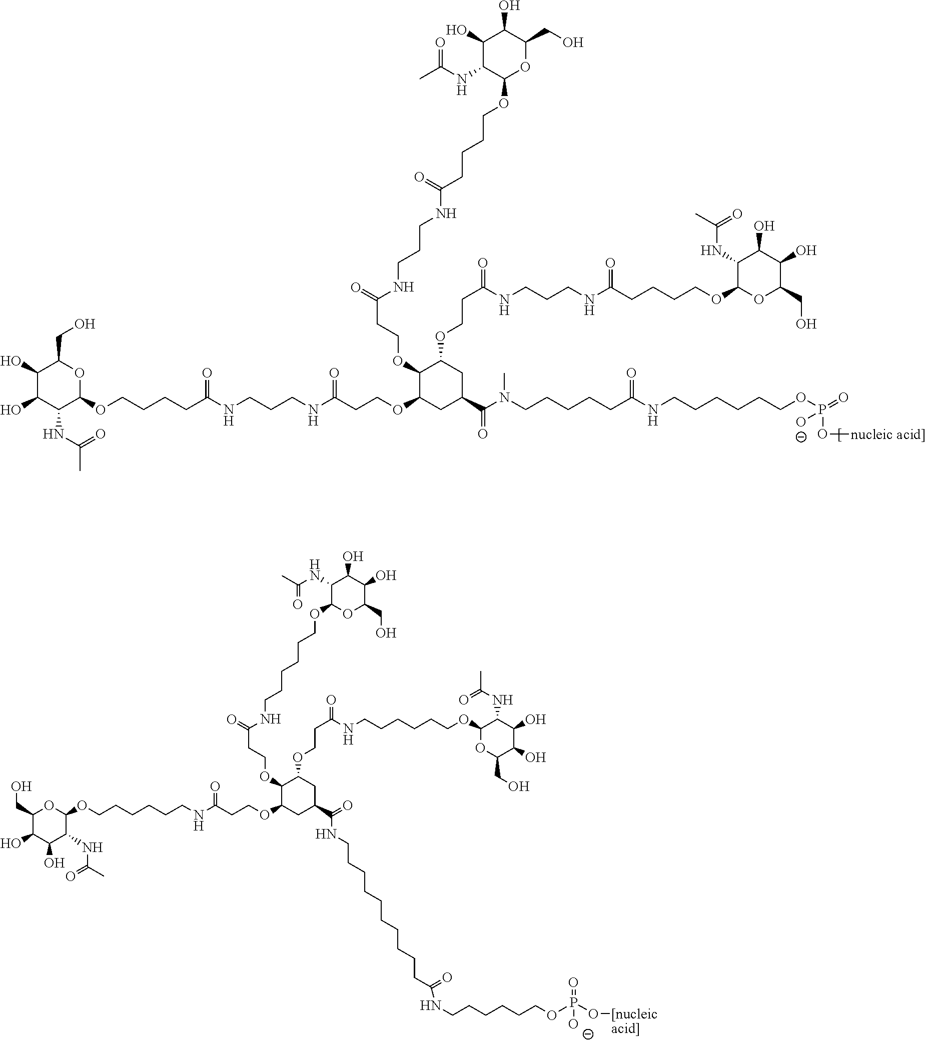

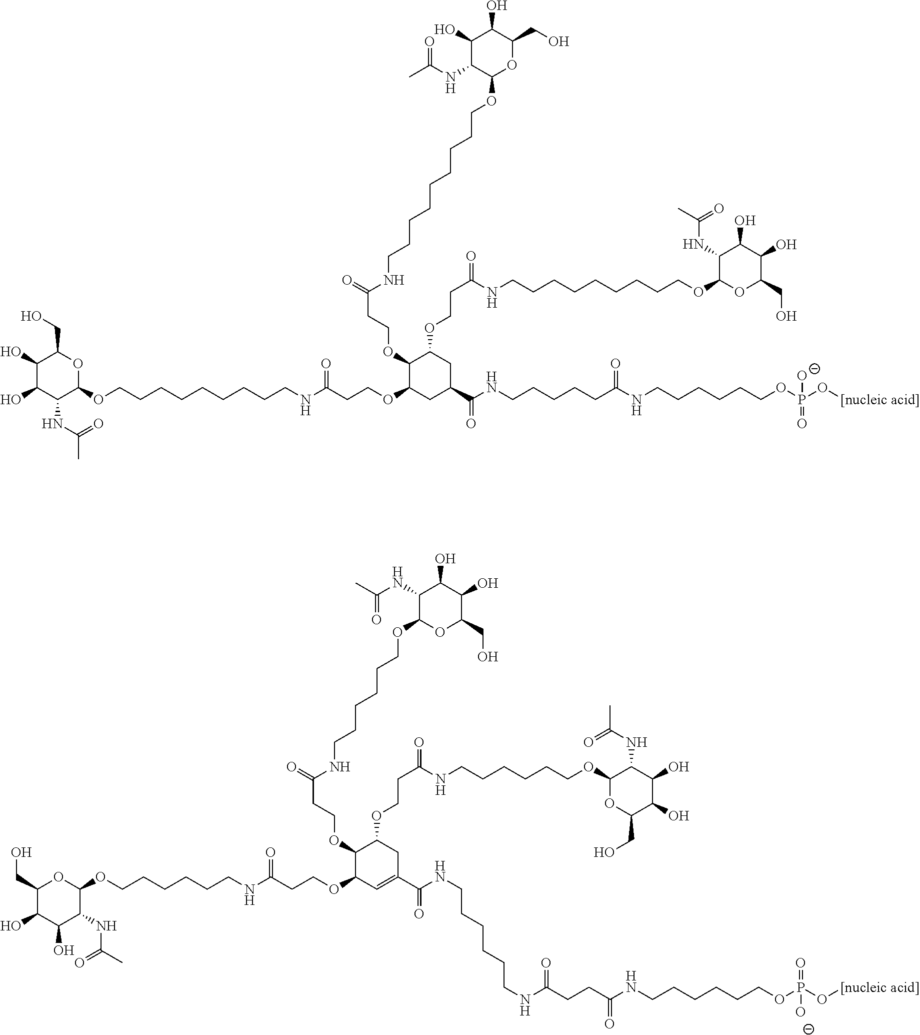

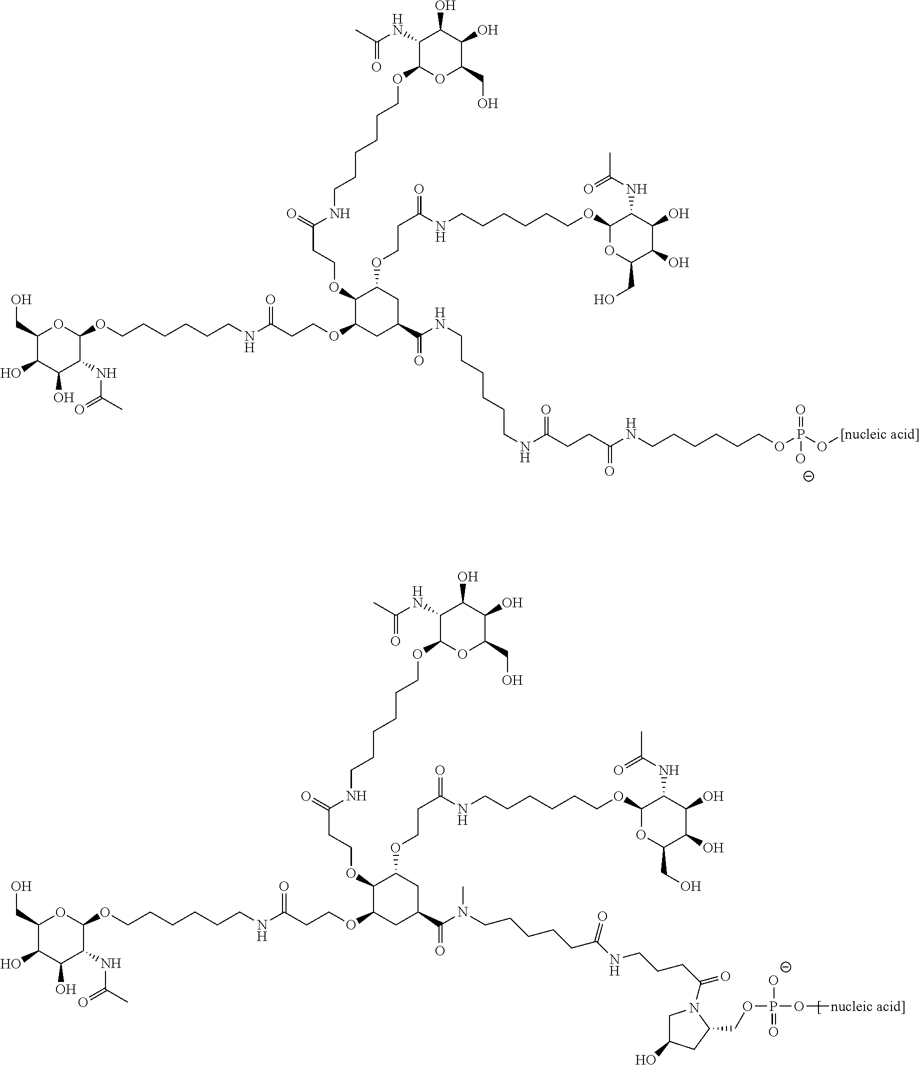

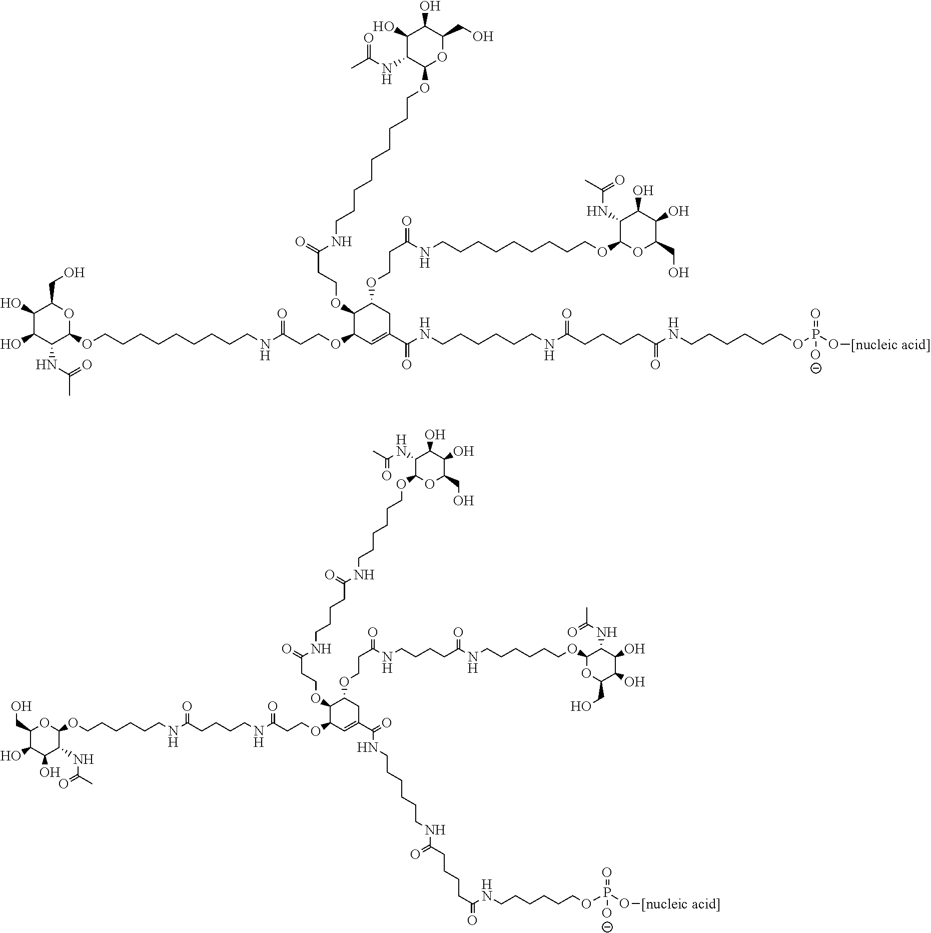

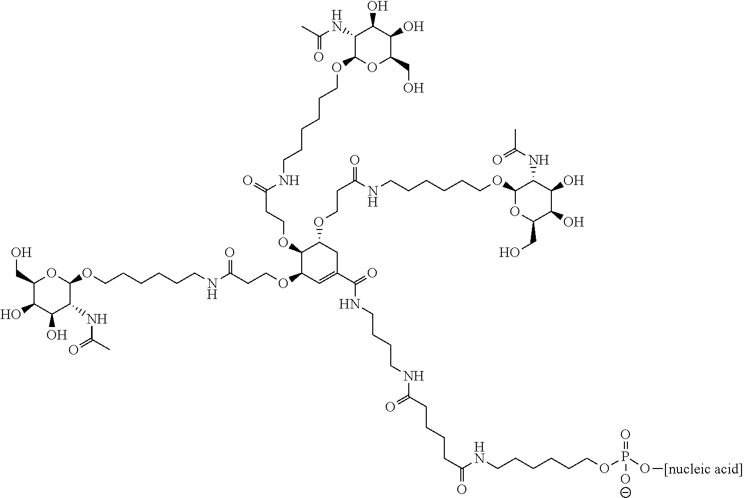

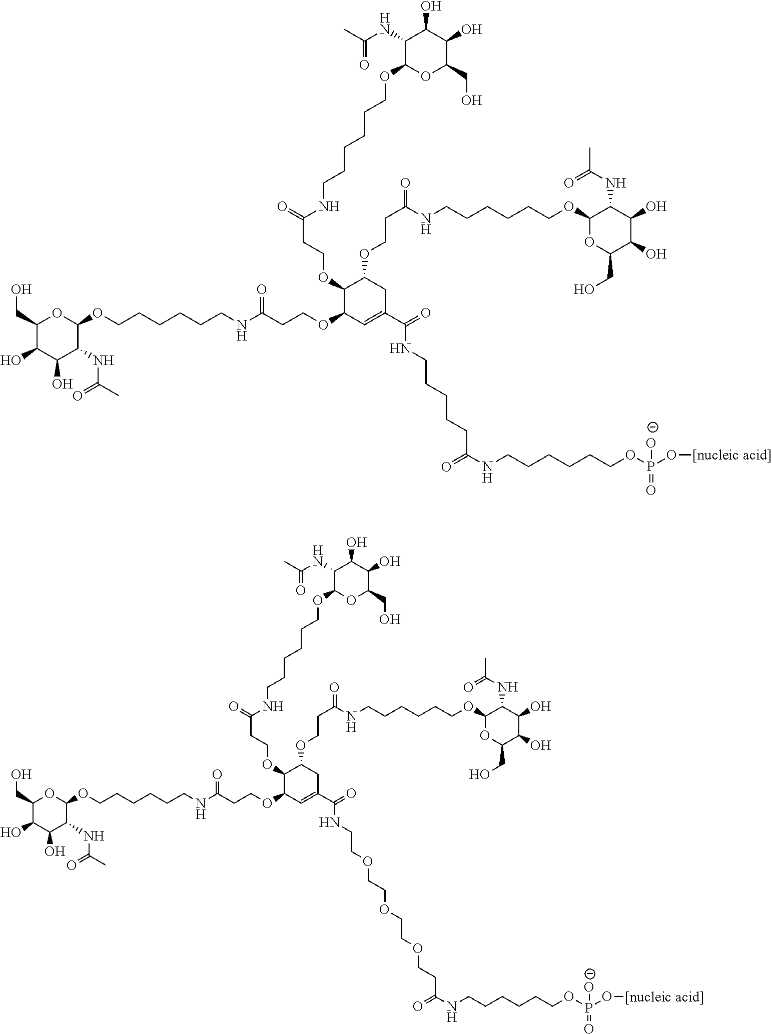

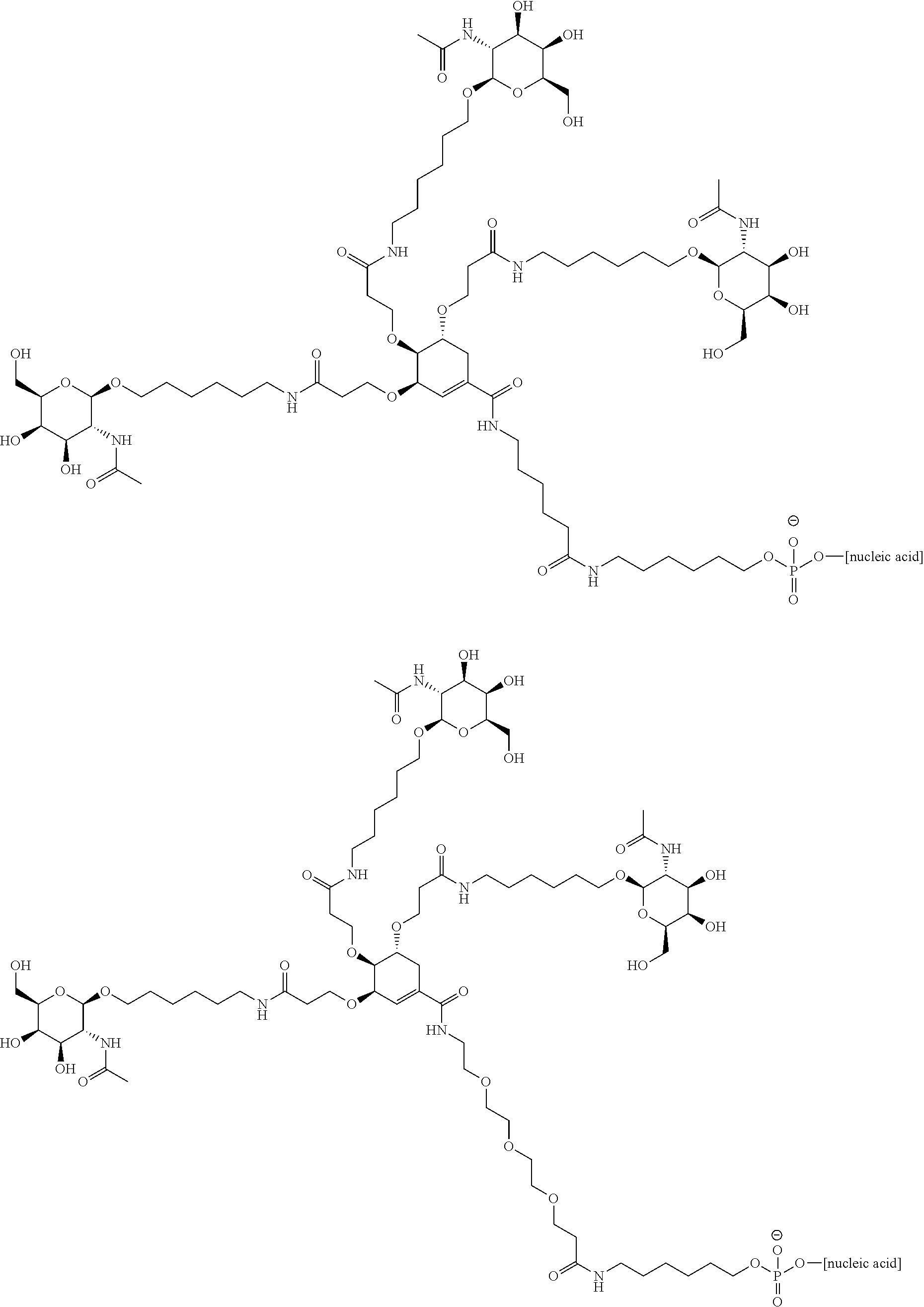

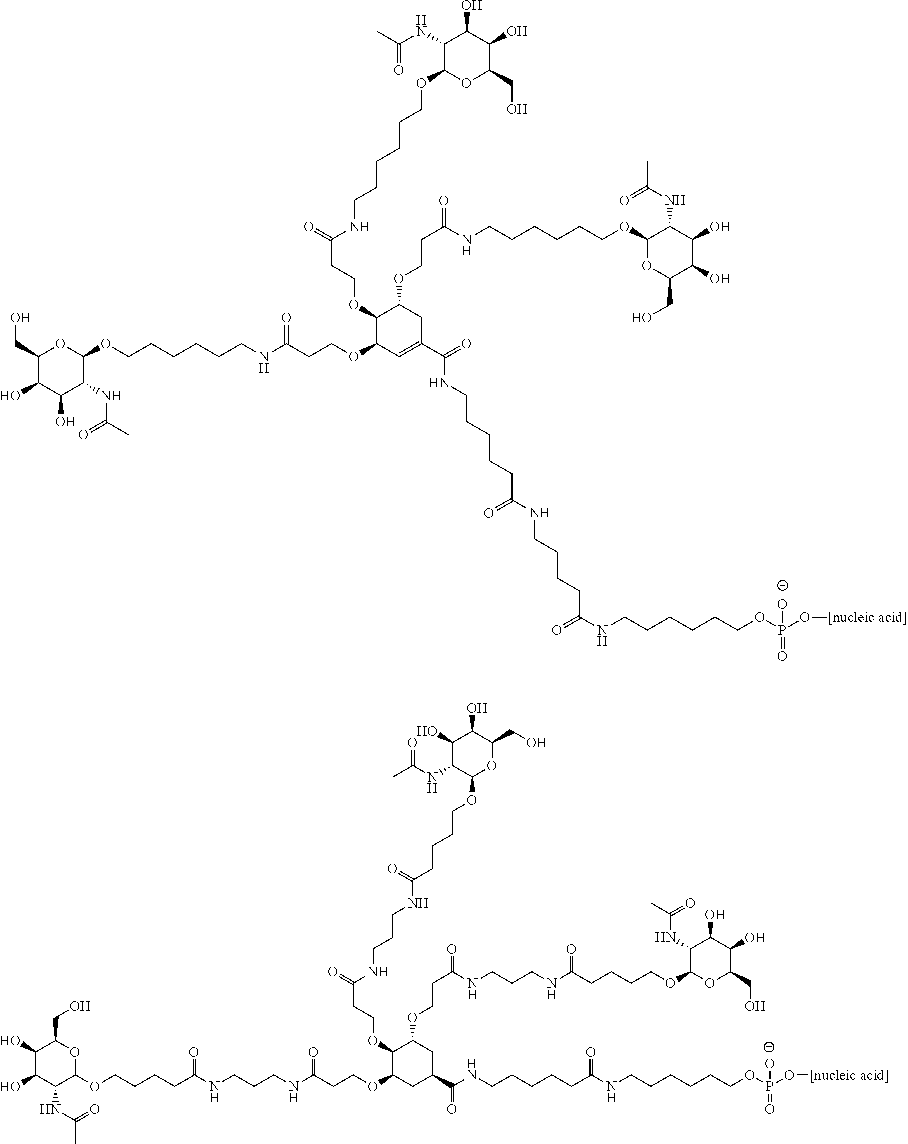

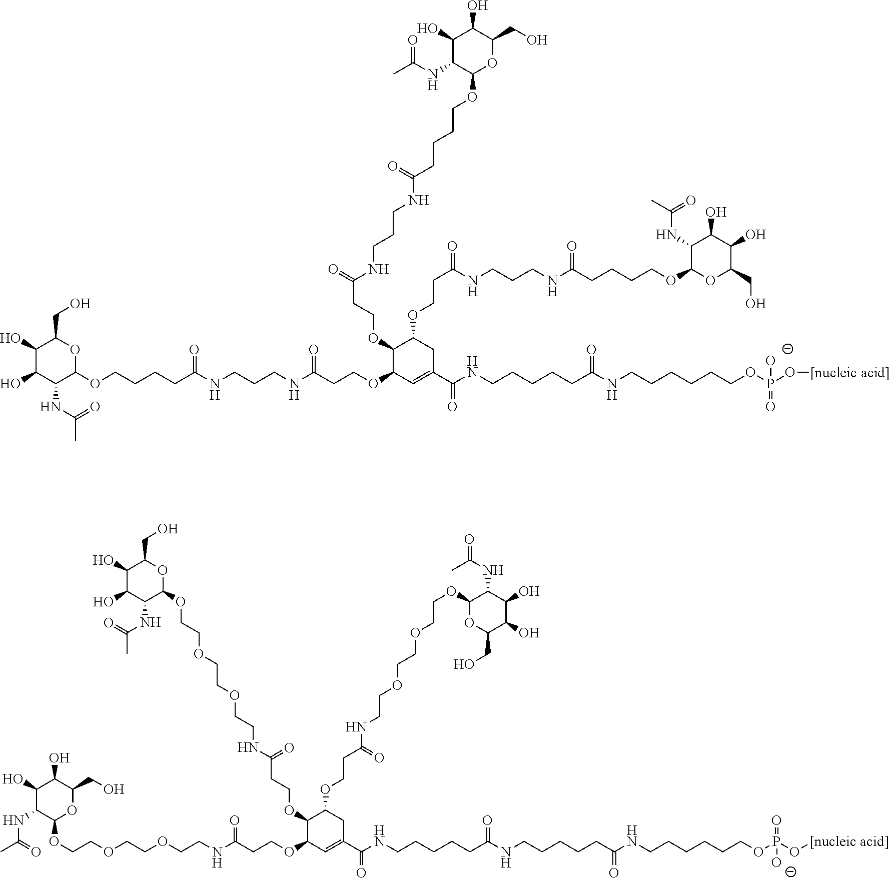

and the pharmaceutically acceptable salts thereof, wherein “nucleic acid” denotes a cargo nucleic acid as defined herein. In embodiments, the nucleic acid is an ASO (e.g., a MALAT1 ASO having a sequence comprising SEQ ID NO: 1). In other embodiments, the nucleic acid is a siRNA. In embodiments, the siRNA is an ANGPTL3 siRNA (e.g., having a sequence comprising SEQ ID NO: 2 and/or SEQ ID NO: 3). In other embodiments, the siRNA is a PPTB siRNA (e.g., having a sequence comprising SEQ ID NO: 4, 5 and/or 6, for example comprising SEQ ID NOs: 4 and 6, or comprising SEQ ID NOs: 5 and 6).

In embodiments, the compound is Compound 1 as defined hereinafter, or a pharmaceutically acceptable salt thereof. In embodiments, the compound is Compound 2a as defined hereinafter, or a pharmaceutically acceptable salt thereof.

In embodiments, the compound is selected from Compounds 1, 2a, 2b, 2c, 3, 4, 5, 6, 7, 8, 9, 10, 11, 12, 13, 14, 15a, 15b, 16a, 16b, 17, 18, 19, 20 and 21 as defined hereinafter, and the pharmaceutically acceptable salts thereof. In embodiments, the compound is selected from Compounds 1, 2a, 2b, 2c, 3, 5, 6, 7, 8, 9, 10, 11, 12, 13, 14, 15a, 15b, 16a, 16b, 17, 18 and 19 as defined hereinafter, and the pharmaceutically acceptable salts thereof.

In embodiments, the compound of the disclosure is characterised according to its binding affinity for ASGPR, e.g., human ASGPR (e.g., as measured according to the FRET assay described in Example 2 below). In embodiments, the compound has an IC₅₀ value of less than about 10 nM, e.g., an IC₅₀ value of less than about 9 nM, 8 nM, 7 nM, 6 nM, 5 nM, 4 nM, 3 nM, or 2 nM. In embodiments, the compound has an IC₅₀ value of less than about 2.0 nM, e.g. less than about 1.5 nM, 1.0 nM or 0.5 nM.

In embodiments, the compound of the disclosure is characterised according to its binding kinetics for ASGPR, e.g., human ASGPR (e.g., as measured according to the SPR assay described in Example 2 below). In embodiments, the compound has a Kd value of less than about 8 nM, e.g., a Kd value of less than about 6 nM, 5 nM, 4 nM, 3 nM, 2 nM, or 1 nM. In embodiments, the compound has a Kd value of less than about 1.5 nM, e.g. less than about 1.0 nM or 0.5 nM. In embodiments, the compound has a Kd value of less than about 20 nM, e.g., a Kd value of less than about 16 nM, 12 nM, or 10 nM.

In embodiments, the compound of the disclosure is characterised according to its binding affinity for native ASGPR, e.g., murine ASGPR (e.g., as measured according to the fluorescence polarization assay described in Example 2 below). In embodiments, the compound has an IC₅₀ value of less than about 10 nM, e.g., an IC₅₀ value of less than about 9 nM, 8 nM, 7 nM, 6 nM, 5 nM, 4 nM, 3 nM, or 2 nM. In embodiments, the compound has an IC₅₀ value of less than about 2.0 nM, e.g. less than about 1.5 nM or 1.0 nM. In embodiments, the compound has an IC₅₀ value of less than about 0.5 nM.

In embodiments, the compound of the disclosure is characterised according to its activity in knocking down gene expression in HEK293 cells, e.g. in HEK293 cells overexpressing ASGPR (e.g., as measured according to the qPCR described Example 2 below). In embodiments, the compound has an IC₅₀ value of less than about 1000 nM, e.g., an IC₅₀ value of less than about 800 nM, 700 nM, 600 nM or 500 nM. In other embodiments, the compound has an IC₅₀ value of less than about 50 nM, e.g. an IC₅₀ value of less than about 20 nM or 10 nM. In embodiments, the compound has an IC₅₀ value of less than about 5 nM.

In other embodiments, the compound of the disclosure is characterised according to its activity in knocking down gene expression in PHH cells, e.g. in PHH cells overexpressing ASGPR (e.g., as measured according to the qPCR described Example 2 below). In embodiments, the compound has an IC₅₀ value of less than about 100 nM, e.g., an IC₅₀ value of less than about 80 nM, 70 nM, 60 nM or 50 nM. In other embodiments, the compound has an IC₅₀ value of less than about 50 nM, e.g. an IC₅₀ value of less than about 20 nM or 10 nM.

In embodiments, the compound has an IC₅₀ value of less than about 5 nM.

In embodiments, the compound of the disclosure is characterised according to its activity in knocking down gene expression in tissues such as liver in vivo (e.g., as measured according to the assay described Example 3 below). In embodiments, the compound can knockdown levels of the target mRNA by at least about 40%, e.g. by at least about 45%, 50%, or 55%. In embodiments, the compound can knockdown levels of the target mRNA by at least about 60%, e.g. by at least about 65%, or 70%. In embodiments, the knockdown of at least about 40% is achieved in liver but not in one or more other tissues (e.g., not in kidney).

### Nucleic Acids

As defined herein, the compounds of the disclosure are particularly suitable for delivering nucleic acids to cells and tissues. The “cargo” groups of the compounds disclosed herein are thus nucleic acids. The term “nucleic acid” as used herein includes nucleic acids selected from the group consisting of DNA, RNA, PNA and LNA. The nucleic acid may be a functional nucleic acid, e.g., whereby the functional nucleic acid is selected from the group consisting of mRNA, micro-RNA, shRNA, combinations of RNA and DNA, siRNA, siNA, antisense nucleic acid (e.g., antisense oligonucleotide (ASO)), ribozymes, aptamers and spiegelmers. In embodiments, the nucleic acid is selected from siRNA and ASO. In embodiments, the nucleic acid is siRNA. In other embodiments, the nucleic acid is ASO.

The nucleic acids may be of any length and can have any number of nucleotides such that they are effective for the intended purpose (e.g., RNAi). In embodiments, a siRNA ranges from 15 to 30 nucleotides. The duplex region of a double stranded RNA may range from 15 to 30 nucleotide base pairs using the Watson-crick base pairing. The duplex region may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 base pairs. In embodiments, the nucleic acid has 19 to 23 base pairs. For example, the nucleic acid may be 19, 20, 21, 22 or 23 base pairs in length. A double stranded RNAi may be blunt ended at one end or on both ends. A double stranded RNAi may have overhangs of 1 or more nucleotides one or both strands at one or both ends. The overhangs may be 1, 2, 3, 4, 5, 6, 7, 8, 9, or 10 nucleotides in length.

For any of the above aspects, the nucleic acid may be a modified nucleic acid. The modification may be selected from substitutions or insertions with analogues of nucleic acids or bases and chemical modification of the base, sugar or phosphate moieties. For example, the nucleic acid may: a) be blunt ended at both ends; b) have an overhang at one end and a blunt end at the other; or c) have an overhang at both ends. One or more nucleotides on the first and/or second strand may be modified, to form modified nucleotides. One or more of the odd numbered nucleotides of the first strand may be modified. One or more of the even numbered nucleotides of the first strand may be modified by at least a second modification, wherein the at least second modification is different from the modification on the one or more add nucleotides. At least one of the one or more modified even numbered nucleotides may be adjacent to at least one of the one or more modified odd numbered nucleotides. The nucleic acid of the disclosure may have the modified nucleotides of the first strand shifted by at least one nucleotide relative to the unmodified or differently modified nucleotides of the second strand.

A nucleic acid of the disclosure may comprise two strands comprising nucleotides, that is able to interfere with gene expression. Inhibition may be complete or partial and can result in downregulation of gene expression in a targeted manner. The nucleic acid may comprise two separate polynucleotide strands; the first strand, which may also be a guide strand; and a second strand, which may also be a passenger strand. The first strand and the second strand may be part of the same polynucleotide molecule that is self-complementary which ‘folds’ to form a double stranded molecule. The nucleic acid may be an siRNA molecule. The first strand may also be referred to as an antisense strand. The second strand may also be referred to as a sense strand.

The nucleic acid may comprise ribonucleotides, modified ribonucleotides, deoxynucleotides, deoxyribonucleotides, or nucleotide analogous. The nucleic acid may further comprise a double stranded nucleic acid portion or duplex region formed by all or a portion of the first strand (also known in the art as a guide strand) and all or a portion of the second strand (also known in the art as a passenger strand). The duplex region is defined as beginning with the first base pair formed between the first strand and the second strand and ending with the last base pair formed between the first strand and the second strand, inclusive.

Depending on the length of a nucleic acid, a perfect match in terms of base complementarity between the first strand and second strand is not necessarily required. However, the first and second strands must be able to hybridise under physiological conditions. The complementarity between the first strand and second strand in the at least one duplex region may be perfect in that there are no nucleotide mismatches or additional/deleted nucleotides in either strand. Alternatively, the complementarity may not be perfect. The complementarity may be at least 70%, 75%, 80%, 85%, 90% or 95%. The first strand and the second strand may each comprise a region of complementarity which comprises at least 15 contiguous nucleotides differing by no more than 3 nucleotides.

Unmodified polynucleotides, particularly ribonucleotides, may be prone to degradation by cellular nucleases, and, as such, modification and/or modified nucleotides may be included in the nucleic acid of the disclosure. One or more nucleotides on the second and/or first strand of the nucleic acid of the disclosure may be modified. Modifications of the nucleic acid of the present disclosure generally provide a powerful tool in overcoming potential limitations including, but not limited to, in vitro and in vivo stability and bioavailability inherent to native RNA molecules. The nucleic acid according to the disclosure may be modified by chemical modifications. Modified nucleic acid can also minimise the possibility of inducing interferon activity in humans. Modification can further enhance the functional delivery of a nucleic acid to a target cell. The modified nucleic acid of the present disclosure may comprise one or more chemically modified ribonucleotides of either or both of the first strand or the second strand. A ribonucleotide may comprise a chemical modification of the base, sugar or phosphate moieties. The ribonucleic acid may be modified by substitution or insertion with analogues of nucleic acids or bases.

One or more nucleotides of a nucleic acid of the present disclosure may be modified. The nucleic acid may comprise at least one modified nucleotide. The modified nucleotide may be on the first strand. The modified nucleotide may be in the second strand. The modified nucleotide may be in the duplex region. The modified nucleotide may be outside the duplex region, i.e., in a single stranded region. The modified nucleotide may be on the first strand and may be outside the duplex region. The modified nucleotide may be on the second strand and may be outside the duplex region. The 3′-terminal nucleotide of the first strand may be a modified nucleotide. The 3′-terminal nucleotide of the second strand may be a modified nucleotide. The 5′-terminal nucleotide of the first strand may be a modified nucleotide. The 5′-terminal nucleotide of the second strand may be a modified nucleotide.

A nucleic acid of the disclosure may have 1 modified nucleotide or a nucleic acid of the disclosure may have about 2-4 modified nucleotides, or a nucleic acid may have about 4-6 modified nucleotides, about 6-8 modified nucleotides, about 8-10 modified nucleotides, about 10-12 modified nucleotides, about 12-14 modified nucleotides, about 14-16 modified nucleotides about 16-18 modified nucleotides, about 18-20 modified nucleotides, about 20-22 modified nucleotides, about 22-24 modified nucleotides, 24-26 modified nucleotides or about 26-28 modified nucleotides. In each case the nucleic acid comprising said modified nucleotides retains at least 50% of its activity as compared to the same nucleic acid but without said modified nucleotides. The nucleic acid may retain 55%, 60%, 65%, 70%, 75%, 80%, 85%, 90%, 95% or 100% or above of its activity as compared to the same nucleic acid but without said modified nucleotides

The modified nucleotide may be a purine or a pyrimidine. At least half of the purines may be modified. At least half of the pyrimidines may be modified. All of the purines may be modified. All of the pyrimidines may be modified. The modified nucleotides may be selected from the group consisting of a 3′ terminal deoxy thymine (dT) nucleotide, a 2′ O methyl modified nucleotide, a 2′ modified nucleotide, a 2′ deoxy modified nucleotide, a locked nucleotide, an abasic nucleotide, a 2′ amino modified nucleotide, a 2′ alkyl modified nucleotide, a morpholino nucleotide, a phosphoramidate, a non-natural base comprising nucleotide, a nucleotide comprising a 5′-phosphorothioate group, a nucleotide comprising a 5′ phosphate or 5′ phosphate mimic and a terminal nucleotide linked to a cholesteryl derivative or a dodecanoic acid bisdecylamide group.

The nucleic acid may comprise a modified nucleotide, wherein the base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methyl cytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetyl cytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N⁶-methyladenosine (m⁶A), 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N⁶-isopentenyladenosine, beta-D-mannosylqueosine, uridine-5-oxyacetic acid and 2-thiocytidine.

Nucleic acids discussed herein include unmodified RNA as well as RNA which has been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. Modified nucleotide as used herein refers to a nucleotide in which one or more of the components of the nucleic acid, namely sugars, bases, and phosphate moieties, are different from that which occur in nature. While they are referred to as modified nucleotides they will of course, because of the modification, include molecules which are not nucleotides, for example a polynucleotide molecule in which the ribophosphate backbone is replaced with a non-ribophosphate construct that allows hybridisation between strands, i.e., the modified nucleotides mimic the ribophosphate backbone.

Many of the modifications described herein that occur within a nucleic acid will be repeated within a polynucleotide molecule, such as a modification of a base, or a phosphate moiety, or the a non-linking oxygen of a phosphate moiety. In some cases the modification will occur at all of the possible positions/nucleotides in the polynucleotide but in many cases it will not. A modification may only occur at a 3′ or 5′ terminal position, may only occur in terminal regions, such as at a position on a terminal nucleotide or in the last 2, 3, 4, 5, or 10 nucleotides of a strand. A modification may occur in a double strand region, a single strand region, or in both. A modification may occur only in the double strand region of a nucleic acid of the disclosure or may only occur in a single strand region of a nucleic acid of the disclosure. A phosphorothioate modification at a non-linking oxygen position may only occur at one or both termini, may only occur in a terminal region, e.g., at a position on a terminal nucleotide or in the last 2, 3, 4 or 5 nucleotides of a strand, or may occur in duplex and/or in single strand regions, particularly at termini. The 5′ end or 3′ ends may be phosphorylated. Stability of a nucleic acid of the disclosure may be increased by including particular bases in overhangs, or to include modified nucleotides, in single strand overhangs, e.g., in a 5′ or 3′ overhang, or in both. Purine nucleotides may be included in overhangs. All or some of the bases in a 3′ or 5′ overhang may be modified. Modifications can include the use of modifications at the 2′ OH group of the ribose sugar, the use of deoxyribonucleotides, instead of ribonucleotides, and modifications in the phosphate group, such as phosphorothioate modifications. Overhangs need not be homologous with the target sequence.

Nucleases can hydrolyse nucleic acid phosphodiester bonds. However, chemical modifications to nucleic acids can confer improved properties, and can render oligoribonucleotides more stable to nucleases. Modified nucleic acids, as used herein, can include one or more of:
(i) alteration, e.g., replacement, of one or both of the non-linking phosphate oxygens and/or of one or more of the linking phosphate oxygens (referred to as linking even if at the 5′ and 3′ terminus of the nucleic acid of the disclosure);
(ii) alteration, e.g., replacement, of a constituent of the ribose sugar, e.g., of the 2′ hydroxyl on the ribose sugar;
(iii) replacement of the phosphate moiety with “dephospho” linkers;
(iv) modification or replacement of a naturally occurring base;
(v) replacement or modification of the ribose-phosphate backbone;
(vi) modification of the 3′ end or 5′ end of the RNA, e.g., removal, modification or replacement of a terminal phosphate group or conjugation of a moiety, e.g., a fluorescently labelled moiety, to either the 3′ or 5′ end of RNA.

The terms replacement, modification, alteration, indicate a difference from a naturally occurring molecule.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. One, each or both non-linking oxygens in the phosphate group can be independently any one of S, Se, B, C, H, N, or OR (R is alkyl or aryl). The phosphate linker can also be modified by replacement of a linking oxygen with nitrogen (bridged phosphoroamidates), sulphur (bridged phosphorothioates) and carbon (bridged methylenephosphonates). The replacement can occur at a terminal oxygen. Replacement of the non-linking oxygens with nitrogen is possible.

A modified nucleotide can include modification of the sugar groups. The 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)˜CH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). “Deoxy” modifications include hydrogen halo; amino (e.g., NH₂; alkylamino, dialkylamino, heterocyclyl, arylamino, diaryl amino, heteroaryl amino, diheteroaryl amino, or amino acid); NH(CH₂CH₂NH)˜CH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Other substituents of certain embodiments include 2′-methoxyethyl, 2′-OCH3, 2′—O-allyl, 2′-C-allyl, and 2′-fluoro.

The sugar group can also contain one or more carbons that possess the opposite stereochemical configuration than that of the corresponding carbon in ribose. Thus, a modified nucleotides may contain a sugar such as arabinose. Modified nucleotides can also include “abasic” sugars, which lack a nucleobase at C-I′. These abasic sugars can further contain modifications at one or more of the constituent sugar atoms.

The 2′ modifications may be used in combination with one or more phosphate linker modifications (e.g., phosphorothioate). The phosphate group can be replaced by non-phosphorus containing connectors. Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In certain embodiments, replacements may include the methylenecarbonylamino and methylenemethylimino groups.

The phosphate linker and ribose sugar may be replaced by nuclease resistant nucleotides. Examples include the mophilino, cyclobutyl, pyrrolidine and peptide nucleic acid (PNA) nucleoside surrogates. In certain embodiments, PNA surrogates may be used.

The 3′ and 5′ ends of an oligonucleotide can be modified. Such modifications can be at the 3′ end or the 5′ end or both ends of the molecule. They can include modification or replacement of an entire terminal phosphate or of one or more of the atoms of the phosphate group. For example, the 3′ and 5′ ends of an oligonucleotide can be conjugated to other functional molecular entities such as labelling moieties, e.g., fluorophores (e.g., pyrene, TAMRA, fluorescein, Cy3 or Cy5 dyes) or protecting groups (based, e.g., on sulphur, silicon, boron or an ester). The functional molecular entities can be attached to the sugar through a phosphate group and/or a linker. The terminal atom of the linker can connect to or replace the linking atom of the phosphate group or the C-3′ or C-5′ O, N, S or C group of the sugar.

Alternatively, the linker can connect to or replace the terminal atom of a nucleotide surrogate (e.g., PNAs). These spacers or linkers can include, e.g., —(CH₂)ₙ-, —(CH₂)—NH—, —(CH₂)˜O-, —(CH₂)˜S-, O(CH₂CH₂O)˜CH₂CH₂OH (e.g., where n=3 or 6), abasic sugars, amide, carboxy, amine, oxyamine, oxyimine, thioether, disulfide, thiourea, sulfonamide, or morpholino, or biotin and fluorescein reagents. The 3′ end can be an —OH group.

Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabelled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including to modulate activity or to modulate resistance to degradation. Terminal modifications useful for modulating activity include modification of the 5′ end with phosphate or phosphate analogs. Nucleic acids of the disclosure, on the first or second strand, may be 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate); (HO)(HS)(S)P—O-5′), 5′-phosphorothiolate ((HO)₂(O)P—S-5′); any additional combination of oxygen/sulphur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (OH)₂(O)P-5′-CH₂—), 5′-vinylphosphonate, 5′-alkyletherphosphonates (R=alkylether=methoxymethyl (MeOCH₂—), ethoxymethyl, etc., e.g., RP(OH)(O)—O-5′-).

The nucleic acid of the present disclosure may include one or more phosphorothioate modifications on one or more of the terminal ends of the first and/or the second strand. Optionally, each or either end of the first strand may comprise one or two or three phosphorothioate modified nucleotides. Optionally, each or either end of the second strand may comprise one or two or three phosphorothioate modified nucleotides. Optionally, both ends of the first strand and the 5′ end of the second strand may comprise two phosphorothioate modified nucleotides. By phosphorothioate modified nucleotide it is meant that the linkage between the nucleotide and the adjacent nucleotide comprises a phosphorothioate group instead of a standard phosphate group.

Terminal modifications can also be useful for monitoring distribution, and in such cases the groups to be added may include fluorophores, e.g., fluorescein or an Alexa dye. Terminal modifications can also be useful for enhancing uptake, useful modifications for this include cholesterol. Terminal modifications can also be useful for cross-linking an RNA agent to another moiety.

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrrolidines, 6-azapyrimidines and N-2, N-6 and 0-6 substituted purines, including 2-aminopropyladenine, 5-propynyluracil and 5-propynyl cytosine, dihydrouracil, 3-deaza-5-azacytosine, 2-aminopurine, 5-alkyluracil, 7-alkylguanine, 5-alkyl cytosine, 7-deazaadenine, N⁶,N⁶-dimethyladenine, 2,6-diaminopurine, 5-amino-allyl-uracil, N³-methyl uracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N<4>-acetyl cytosine, 2-thiocytosine, N⁶-methyladenine, N⁶-isopentyladenine, 2-methylthio-N⁶-isopentenyladenine, N-methylguanines, or O-alkylated bases.

Certain moieties may be linked to the 5′ terminus of the first strand or the second strand and includes abasic ribose moiety, abasic deoxyribose moiety, modifications abasic ribose and abasic deoxyribose moieties including 2′ O-alkyl modifications; inverted abasic ribose and abasic deoxyribose moieties and modifications thereof, C6-imino-Pi; a mirror nucleotide including L-DNA and L-RNA; 5′ OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non-bridging methylphosphonate and 5′-mercapto moieties. The nucleic acids of the disclosure may include one or more inverted nucleotides, for example inverted thymidine or inverted adenine (see, e.g., Takei et al., J Biol Chem (2002) 277(26):23800-23806).

The nucleic acid of the present disclosure may comprise an abasic nucleotide. The nucleic acid may comprise one or more nucleotides on the second and/or first strands that are modified. Alternating nucleotides may be modified, to form modified nucleotides. In alternating nucleotides, one nucleotide may be modified with a first modification, the next contiguous nucleotide may be modified with a second modification and the following contiguous nucleotide is modified with the first modification and so on, where the first and second modifications are different.

### RNA Modifications

Modifications of the siRNA molecules of the present disclosure generally provides a powerful tool in overcoming potential limitations including, but not limited to, in vitro and in vivo stability and bioavailability inherent to native RNA molecules. The siRNA according to the disclosure may be modified by chemical modifications. Modified siRNA can also minimize the possibility of activating interferon activity in humans. Modification can further enhance the functional delivery of a siRNA to a target cell. The modified siRNA of the present disclosure may comprise one or more chemically modified ribonucleotides of either or both of the antisense strand or the sense strand. A ribonucleotide may comprise a chemical modification of the base, sugar or phosphate moieties. The ribonucleic acid may be modified by substitution or insertion with analogues of nucleic acids or bases.

One or more nucleotides of a siRNA of the present disclosure may comprise a modified base. In one aspect, the siRNA comprises at least one nucleotide comprising a modified base. In one embodiment, the modified base in on the antisense strand. In another embodiment, the modified base in on the sense strand. In another embodiment, the modified base is in the duplex region. In another embodiment, the modified base is outside the duplex region, i.e., in a single stranded region. In another embodiment, the modified base is on the antisense strand and is outside the duplex region. In another embodiment, the modified base is on the sense strand and is outside the duplex region. In another embodiment, the 3′-terminal nucleotide of the antisense strand is a nucleotide with a modified base. In another embodiment, the 3′-terminal nucleotide of the sense strand is nucleotide with a modified base. In another embodiment, the 5′-terminal nucleotide of the antisense strand is nucleotide with a modified base. In another embodiment, the 5′-terminal nucleotide of the sense strand is nucleotide with a modified base.

The modified base may be a purine or a pyrimidine. In another embodiment, at least half of the purines are modified. In another embodiment, at least half of the pyrimidines are modified. In another embodiment, all of the purines are modified. In another embodiment, all of the pyrimidines are modified. In another embodiment, the siRNA may comprise a nucleotide comprising a modified base, wherein the base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid and 2-thiocytidine.

In another aspect, a siRNA of the present disclosure comprises an abasic nucleotide. As used herein, a nucleotide with a modified base does not include abasic nucleotides.

### Modifications to Sugar Moiety

Another aspect relates to modifications to a sugar moiety. One or more nucleotides of a siRNA of the present disclosure may comprise a modified ribose moiety. Modifications at the 2′-position where the 2′-OH is substituted include the non-limiting examples selected from alkyl, substituted alkyl, alkaryl-, arylalkyl-, —F, —Cl, —Br, —CN, —CF₃, —OCF₃, —OCN, —O-alkyl, —S-alkyl, HS-alkyl-O, —O-alkenyl, —S-alkenyl, —N-alkenyl, —SO-alkyl, -alkyl-OSH, -alkyl-OH, —O-alkyl-OH, —O-alkyl-SH, —S-alkyl-OH, —S-alkyl-SH, -alkyl-S-alkyl, -alkyl-O-alkyl, —ONO₂, —NO₂, —N₃, —NH₂, alkylamino, dialkylamino-, aminoalkyl-, aminoalkoxy, aminoacid, aminoacyl-, —ONH₂, —O-aminoalkyl, —O-aminoacid, —O-aminoacyl, heterocycloalkyl-, heterocycloalkaryl-, aminoalkylamino-, polyalklylamino-, substituted silyl-, methoxyethyl-(MOE), alkenyl and alkynyl. “Locked” nucleic acids (LNA) in which the 2′ hydroxyl is connected, e.g., by a methylene bridge, to the 4′ carbon of the same ribose sugar is further included as a 2′ modification of the present disclosure. In embodiments, substituents are 2′-methoxyethyl, 2′-O—CH₃, 2′—O-allyl, 2′-C-allyl, and 2′-fluoro (2′-F).

### Modified Groups

In one aspect, the antisense duplex region comprises a plurality of groups of modified nucleotides, referred to herein as “modified groups”, wherein each modified group consists of one or more identically modified nucleotides, wherein each modified group is flanked on one or both sides by a second group of nucleotides, referred to herein as “flanking groups”, wherein each said flanking group consists of one or more nucleotides that are either unmodified or modified in a manner different from the nucleotides of said modified group. In one embodiment, each modified group in the antisense duplex region is identical, i.e., each modified group consists of an equal number of identically modified nucleotides. In another embodiment, each flanking group has an equal number of nucleotides. In another embodiment, each flanking group is identical. In another embodiment, the nucleotides of said modified groups in the antisense duplex region comprise a modified base. In another embodiment, the nucleotides of said modified groups comprise a modified phosphate backbone. In another embodiment, the nucleotides of said modified groups comprise a modified 2′ position.

In another aspect, the sense duplex region comprises a plurality of groups of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the sense duplex region is identical. In another embodiment, each flanking group has an equal number of nucleotides. In another embodiment, each flanking group is identical. In another embodiment, the nucleotides of said modified groups in the sense duplex region comprise a modified base. In another embodiment, the nucleotides of said modified groups comprise a modified phosphate backbone. In another embodiment, the nucleotides of said modified groups comprise a modified 2′ position.

In another aspect, the antisense duplex region and the sense duplex region each comprise a plurality of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the antisense duplex region and the sense duplex region are identical. In another embodiment, each flanking group in the antisense duplex region and the sense duplex region each have an equal number of nucleotides. In another embodiment, each flanking group in the antisense duplex region and in the sense duplex region are identical. In another embodiment, the nucleotides of said modified groups in the antisense duplex region and the sense duplex region each comprise the same modified groups and the same flanking groups. In another embodiment, the nucleotides of said modified groups in the antisense duplex region and the sense duplex region each comprise a modified base. In another embodiment, the nucleotides of said modified groups in the antisense duplex region and the sense duplex region each comprise a modified phosphate backbone. In another embodiment, the nucleotides of said modified groups in the antisense duplex region and the sense duplex region each comprise a modified 2′ position.

### Modifications to the Phosphate Backbone

Another aspect relates to modifications to a phosphate backbone. All or a portion of the nucleotides of the siRNA of the disclosure may be linked through phosphodiester bonds, as found in unmodified nucleic acid. A siRNA of the present disclosure however, may comprise a modified phosphodiester linkage. The phosphodiester linkages of either the antisense stand or the sense strand may be modified to independently include at least one heteroatom selected from nitrogen and sulphur. In one embodiment, a phosphoester group connecting a ribonucleotide to an adjacent ribonucleotide is replaced by a modified group. In one embodiment, the modified group replacing the phosphoester group is selected from phosphorothioate, methylphosphonate, phosphorodithioate or phosphoramidate group.

### 5′ and 3′ End Modifications

The siRNA of the present disclosure may include nucleic acid molecules comprising one or more modified nucleotides, abasic nucleotides, acyclic or deoxyribonucleotide at the terminal 5′- or 3′-end on either or both of the sense or antisense strands. The 5′-end nucleotide of the antisense and/or strand may be phosphorylated. In another embodiment, the 5′-end nucleotide of the antisense strand is phosphorylated and the 5′-end nucleotide of the sense strand has a free hydroxyl group (5′-OH). In another embodiment, the 5′-end nucleotide of the antisense strand is phosphorylated and the 5′-end nucleotide of the sense strand is modified. In another embodiment the 5′-end nucleotide of the antisense strand carries a 5′E vinylphosphonate.

Modifications to the 5′- and 3′-end nucleotides are not limited to the 5′ and 3′ positions on these terminal nucleotides. Examples of modifications to end nucleotides include, but are not limited to, biotin, inverted (deoxy) abasics, amino, fluoro, chloro, bromo, CN, CF, methoxy, imidazole, carboxylate, thioate, C₁-C₁₀ lower alkyl, substituted lower alkyl, alkaryl or arylalkyl, OCF₃, OCN, O—, S—, or N-alkyl; O—, S—, or N-alkenyl; SO—CH₃; SO₂CH₃; ONO₂; NO₂, N₃; heterocycloalkyl; heterocycloalkaryl; aminoalkylamino; polyalkylamino or substituted silyl, as, among others, described, e.g., in PCT patent publication WO 99/54459, or in European patent EP 0 586 520 B1 or EP 0 618 925 B1, each of which is incorporated by reference in its entirety.

In another embodiment, the terminal 3′ nucleotide or two terminal 3′-nucleotides on either or both of the antisense strand or sense strand is a 2′-deoxynucleotide. In another embodiment, the 2′-deoxynucleotide is a 2′-deoxy-pyrimidine. In another embodiment, the 2′-deoxynucleotide is a 2′ deoxy-thymidine.

shRNA (short hairpin loop RNA) and linked siRNA

Another aspect relates to shRNA and linked siRNA. The antisense strand and the sense strand may be covalently linked to each other. Such linkage may occur between any of the nucleotides forming the antisense strand and sense strand, respectively and can be formed by covalent or non-covalent linkages. Covalent linkage may be formed by linking both strands one or several times and at one or several positions, respectively, by a compound selected from the group comprising methylene blue and bifunctional groups. In embodiments, bifunctional groups are selected from the group comprising bis(2-chloroethyl)amine, N-acetyl-N′-(p-glyoxylbenzoyl)cystamine, 4-thiouracil and psoralene.

Further, the antisense strand and the sense strand may be linked by a loop structure. The loop structure may be comprised of a non-nucleic acid polymer such as polyethylene glycol. The 5′-end of the antisense strand may be linked to the 3′-terminus of the sense strand or the 3′-end of the antisense strand may be linked to the 5′-end of the sense strand. The loop may consist of a nucleic acid, a locked nucleic acid (LNA), a peptide nucleic acid (PNA), or the loop may be formed by polymers. The length of the loop may be sufficient for linking the two strands covalently in a manner that a back folding can occur through a loop structure or similar structure.

The ribonucleic acid constructs may be incorporated into suitable vector systems. In embodiments, the vector comprises a promoter for the expression of RNAi. The promoter may be selected from any known in the art such as, e.g., pol III, U6, H1 or 7SK.

The nucleic acids according to the disclosure may comprise one or more phosphorothioate internucleotide linkages. The phosphorothioate internucleotide linkages may be distributed across the entire nucleotide sequences and may occur in any number at any position. The nucleic acids can comprise between one to ten phosphorothioate internucleotide linkages. The antisense strand may have at least 1 phosphorothioate modification at each end. The antisense strand may have 1-3 phosphorothioate modifications at each end. For example, the antisense strand may have 2 phosphorothioate modifications at each end. The sense strand may have at least 1 phosphorothioate modification at the 3′ end. The sense strand may have 1-3 phosphorothioate modification at the 3′ end. For example, the sense strand may have 2 phosphorothioate modifications at the 3′ end.

siRNA with overhangs

An overhang at the 3′-end or 5′ end of the sense strand or the antisense strand may be selected from the group consisting of 1, 2, 3, 4 and 5 nucleotides in length. Alternatively, the siRNA molecule may be blunt-ended on both ends and may have a length of 16 to 29 consecutive nucleotides. In one embodiment, the siRNA molecule is blunt-ended on one end and the double stranded or duplex portion of the siRNA molecule has a length selected from 16 to 29 consecutive nucleotides. In one embodiment, the siRNA molecule has overhangs on both ends on either strand and the double stranded or duplex portion of the siRNA molecule has a length of 16 to 29 consecutive nucleotides. The overhang may comprise at least one deoxyribonucleotides and/or a TT dinucleotide.

It will be appreciated by one skilled in the art that the modification, modifications of the sugar moiety, pattern, 5′ and 3′ end modifications, overhangs, formulations, delivery, dosage and routes of delivery as described above may equally be applied to any type of RNAi molecule and is not limited to siRNAs.

The nucleic acid of the present disclosure can be produced using routine methods in the art including chemically synthesis or expressing the nucleic acid either in vitro (e.g., run off transcription) or in vivo. For example, using solid phase chemical synthesis or using an expression vector. In one embodiment, the expression vector can produce the nucleic acid of the disclosure in a target cell. Methods for the synthesis and purification of the nucleic acid molecules described herein are known to persons skilled in the art.

### Pharmaceutical Compositions

In one aspect, the disclosure provides a pharmaceutical composition comprising a compound described herein (e.g., a compound of Formula (I)) or a pharmaceutically acceptable salt thereof, and at least one pharmaceutically acceptable excipient or carrier.

In embodiments, the pharmaceutical composition comprises a compound of Formula (I) (e.g., a compound of Formula (Ia) or Formula (Ib)) or a pharmaceutically acceptable salt thereof. In embodiments, the pharmaceutical composition comprises a compound of Formula (II) (e.g., a compound of Formula (IIa) or Formula (IIb)) or a pharmaceutically acceptable salt thereof. In embodiments, the pharmaceutical composition comprises a compound of Formula (III) (e.g., a compound of Formula (IIIa) or Formula (IIIb)) or a pharmaceutically acceptable salt thereof. In embodiments, the pharmaceutical composition comprises a compound of Formula (IV) (e.g., a compound of Formula (IVa) or Formula (IVb)) or a pharmaceutically acceptable salt thereof. In embodiments, the pharmaceutical composition comprises a compound of Formula (V) (e.g., a compound of Formula (Va) or Formula (Vb)) or a pharmaceutically acceptable salt thereof. In embodiments, the pharmaceutical composition comprises a compound of Formula (IX) (e.g., a compound of Formula (IXa)) or a pharmaceutically acceptable salt thereof. In embodiments, the pharmaceutical composition comprises a compound of Formula (XI) (e.g., a compound of Formula (XIa)) or a pharmaceutically acceptable salt thereof. In embodiments, the pharmaceutical composition comprises a compound of Formula (XIII).

Nucleic acids and conjugated compounds can be delivered to cells, both in vitro and in vivo, by a variety of methods known to those skilled in the art, including direct contact with cells or by combination with one or more agents that facilitate targeting and/or delivery into cells. Such agents and methods include lipoplexes, liposomes, iontophoresis, hydrogels, cyclodextrins, nanocapsules, micro- and nanospheres and proteinaceous vectors. The nucleic acid/vehicle combination may be locally delivered in vivo by direct injection or by use of an infusion pump.

The composition of the disclosure may comprise surface-modified liposomes containing poly(ethylene glycol) lipids (PEG-modified, or long-circulating liposomes or stealth liposomes). These formulations offer a method for increasing stability of a liposome or lipoplex solutions by preventing their aggregation and fusion. The formulations also have the added benefit in vivo of resisting opsonization and elimination by the mononuclear phagocytic system (MPS or RES), thereby enabling longer blood circulation times and enhanced tissue exposure for the encapsulated drug. Such liposomes may accumulate selectively in tumors, presumably by extravasation and capture in the neovascularized target tissues. The long-circulating liposomes enhance the pharmacokinetics and pharmacodynamics of DNA and RNA, particularly compared to conventional cationic liposomes which are known to accumulate in tissues of the MPS (see, e.g., Liu et al, J. Biol. Chem. (1995) 42:24864-24780; and PCT publication Nos. WO 96/10391; WO 96/10390; and WO 96/10392, the contents of each of which are incorporated herein in their entirety). Long-circulating liposomes may also protect siRNA from nuclease degradation.

The pharmaceutical compositions of the present disclosure may be used as medicaments or as diagnostic agents. For example, one or more compounds (e.g., siRNA conjugates) of the disclosure can be combined with a delivery vehicle (e.g., liposomes) and excipients, such as carriers or diluents. Other agents such as preservatives and stabilizers can also be added. Methods for the delivery of nucleic acid-containing molecules are known in the art and within the knowledge of the person skilled in the art. The compounds (e.g., siRNA conjugates) of the present disclosure can also be administered in combination with other therapeutic compounds, either administrated separately or simultaneously, e.g., as a combined unit dose.

The pharmaceutical composition may be a sterile injectable aqueous suspension or solution, or in a lyophilized form. In one embodiment, the pharmaceutical composition comprises lyophilized lipoplexes or an aqueous suspension of lipoplexes. In embodiments, the lipoplexes comprise a compound of the disclosure. Such lipoplexes may be used to deliver the compounds of the disclosure to a target cell either in vitro or in vivo.

The pharmaceutical compositions and medicaments of the present disclosure may be administered to a subject (e.g., a mammal) in a pharmaceutically effective dose. The mammal may be selected from humans, dogs, cats, horses, cattle, pig, goat, sheep, mouse, rat, hamster and guinea pig. In embodiments, the subject is human.

A compound or composition of the disclosure (e.g., a composition that includes a double stranded siRNA) can be delivered to a subject by a variety of routes. Exemplary routes include: subcutaneous, intramuscular, intradermal, intravenous, topical, rectal, anal, vaginal, nasal, pulmonary, and ocular. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration. In embodiments, a compound of the disclosure is delivered in vivo by means selected from intravenous, subcutaneous, intramuscular or intradermal injection, or by inhalation. In one embodiment, the compound of the disclosure is delivered by intravenous injection or infusion.

The route and/or site of administration may be chosen to enhance targeting. For example, to target muscle cells, intramuscular injection into the muscles of interest would be a logical choice. Lung cells might be targeted by administering the composition in aerosol form. The vascular endothelial cells could be targeted by coating a balloon catheter with the compound or composition and mechanically introducing the nucleic acid.

The pharmaceutical compositions of the disclosure may be formulated for administration in solid or liquid form, e.g., using conventional carriers or excipients. Compositions may be adapted for, e.g., oral administration (e.g., as a solution, suspension, tablet, or capsule), parenteral administration (e.g., as a solution, dispersion, suspension, or emulsion, or as a dry powder for reconstitution), or topical application (e.g., as a cream, ointment, patch, or spray to be applied to the skin) using techniques known in the art.

### Medical Uses

Compounds of the present disclosure act as modulators of nucleic acids in vivo, which gives them utility in the treatment of numerous disorders and conditions.

Viewed from this aspect, the disclosure provides a compound of the disclosure (or a pharmaceutically acceptable salt thereof, or a pharmaceutical composition comprising the compound or salt) for use in therapy. In a related aspect is provided the use of a compound of the disclosure (or a pharmaceutically acceptable salt thereof, or a pharmaceutical composition comprising the compound or salt) as a medicament. In another related aspect is provided a method of treating a subject in need thereof, the method comprising administering an effective amount of a compound of the disclosure (or a pharmaceutically acceptable salt thereof, or a pharmaceutical composition comprising the compound or salt) to the subject. In another related aspect is provided the use of a compound of the disclosure (or a pharmaceutically acceptable salt thereof, or a pharmaceutical composition comprising the compound or salt) in the manufacture of a medicament.

In particular, compounds of the disclosure can target cells and/or tissues which carry an ASGP receptor and are useful in the treatment of such cells and tissues by delivery of therapeutic nucleic acids. View from this aspect, the disclosure provides a compound of the disclosure (or a pharmaceutically acceptable salt thereof, or a pharmaceutical composition comprising the compound or salt) for use in the treatment of a condition in a subject, which condition is treatable by delivery of a therapeutic nucleic acid to cells and/or tissues of the subject which express ASGPR. In a related aspect, the disclosure provides a method for improving the therapeutic activity of a therapeutic nucleic acid in treating a condition in a subject, which condition is treatable by delivery of the therapeutic nucleic acid to cells and/or tissues of the subject which express ASGPR, the method comprising coupling the therapeutic nucleic acid with one or more molecules to form a compound of the present disclosure (or a pharmaceutically acceptable salt thereof, or a pharmaceutical composition comprising the compound or salt). In another related aspect, the disclosure provides a method for improving the treatment of a condition in a subject, which condition is treatable by delivery of a therapeutic nucleic acid to cells and/or tissues of the subject which express ASGPR, wherein the method comprises delivering the therapeutic nucleic acid as part of a compound of the present disclosure (or a pharmaceutically acceptable salt thereof, or a pharmaceutical composition comprising the compound or salt).

Examples of conditions which may be treatable by delivery of a compound of the disclosure (or a pharmaceutically acceptable salt thereof, or a pharmaceutical composition comprising the compound or salt) include liver diseases (e.g., liver cancer, such as hepatocellular carcinoma), genetic diseases, haemophilia and bleeding disorders, liver fibrosis, non-alcoholic steatohepatitis (NASH), non-alcoholic fatty liver disease (NAFLD), viral hepatitis, rare diseases (e.g., acromegaly), metabolic diseases (e.g. hypercholesterolemia, dyslipidaemia, hypertriglyceridemia), cardiovascular diseases, obesity, thalassemia, liver injury (e.g., drug induced liver injury), hemochromatosis, alcoholic liver diseases, alcohol dependence, anaemia, and anaemia of chronic diseases. In embodiments, the condition is selected from NASH, NAFLD, a metabolic disease and a cardiovascular disease. In embodiments, the condition is NASH.

Viewed from another aspect, the disclosure provides a compound of the disclosure (or a pharmaceutically acceptable salt thereof, or a pharmaceutical composition comprising the compound or salt) for use in the treatment of a condition as defined herein. In a related aspect is provided the use of a compound of the disclosure (or a pharmaceutically acceptable salt thereof, or a pharmaceutical composition comprising the compound or salt) in the treatment of a condition as defined herein. In another related aspect is provided a method of treating a condition in a subject in need thereof, the method comprising administering an effective amount of a compound of the disclosure (or a pharmaceutically acceptable salt thereof, or a pharmaceutical composition comprising the compound or salt) to the subject, wherein the condition is as defined herein. In a further related aspect is provided the use of a compound of the disclosure (or a pharmaceutically acceptable salt thereof, or a pharmaceutical composition comprising the compound or salt) in the manufacture of a medicament for the treatment of a condition as defined herein. In embodiments of the above aspects, the condition is selected from NASH, NAFLD, a metabolic disease (e.g., selected from hypercholesterolemia, dyslipidaemia, and hypertriglyceridemia) and a cardiovascular disease. In embodiments, the condition is NASH.

### Delivery of Nucleic Acids to Cells

A further aspect provides a method of delivering a nucleic acid to a cell using a compound according to the present disclosure (or a pharmaceutically acceptable salt thereof), wherein the cell carries (e.g., expresses) a binding partner for a ligand of the compound. The method comprises the steps of contacting the cell with the compound. The method may be used in vitro (e.g., for diagnostic or research purposes) or in vivo (e.g., for diagnostic or therapeutic purposes). In embodiments, the method is an in vitro method.

In embodiments, the binding partner is ASGPR (e.g., human ASGPR) and the compound carries at least one monosaccharide ligand (e.g., the compound carries at least one GalNAc moiety). In embodiments, the cell is a hepatocyte, e.g. a mammalian hepatocyte such as a human hepatocyte. In embodiments, the cell is a malignant hepatocyte (e.g., a hepatocellular carcinoma cell).

In embodiments, the method comprises contacting a cell which carries (e.g., expresses) ASGPR (e.g., a hepatocyte cell) with a compound which carries at least one GalNAc moiety, e.g. wherein the compound is a nucleic acid delivery agent comprising Formula (II), or wherein the compound is a compound of Formula (V), Formula (VI), Formula (VII) or Formula (VIII), or a pharmaceutically acceptable salt of the foregoing. In embodiments, the method comprises contacting a cell which carries (e.g., expresses) ASGPR (e.g., a hepatocyte cell) with a compound which carries at least one GalNAc moiety, e.g. wherein the compound is a compound of Formula (I), Formula (II), Formula (III), Formula (IV), Formula (V), Formula (IX), Formula (XI), or Formula (XIII), or a pharmaceutically acceptable salt of the foregoing.

### Processes and Intermediates

The disclosure also provides process for the preparation of compounds as described herein, as well as intermediates useful in the preparation of said compounds.

Viewed from this aspect, the disclosure provides a compound having the structure of Formula (VI)

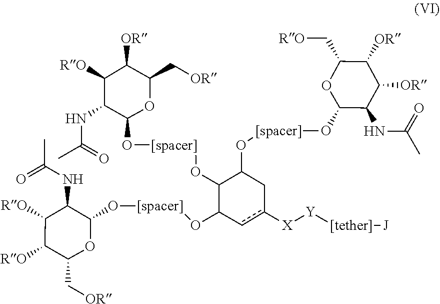

or a pharmaceutically acceptable salt thereof, wherein X, Y, spacer and tether are as defined herein; [symbol] represents a carbon-carbon single bond or double bond, with the proviso that when X is a covalent bond [symbol] represents a carbon-carbon single bond; R″ is acyl, —C(O)aryl or —H; and J is —CO₂H, —OH, —C(O)O-(pentafluorophenyl), —N₃ or a phosphoramidite.

In embodiments, the compound is a compound having the structure of Formula (VIa):

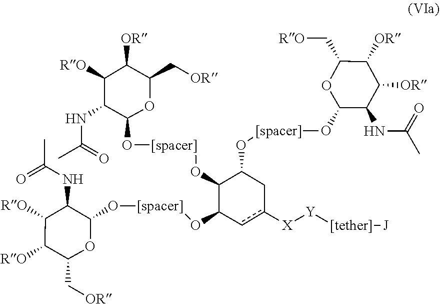

or a pharmaceutically acceptable salt thereof, wherein X, Y, R″, spacer, tether and J are as defined herein, and [symbol] represents a carbon-carbon single bond or double bond, with the proviso that when X is a covalent bond [symbol] represents a carbon-carbon single bond.

In embodiments, the compound is a compound having the structure of Formula (VIb):

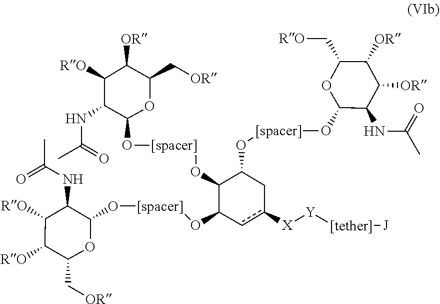

or a pharmaceutically acceptable salt thereof, wherein X, Y, R″, spacer, tether and J are as defined herein, and [symbol] represents a carbon-carbon single bond.

In embodiments, the compound is a compound having the structure of Formula (VIc):

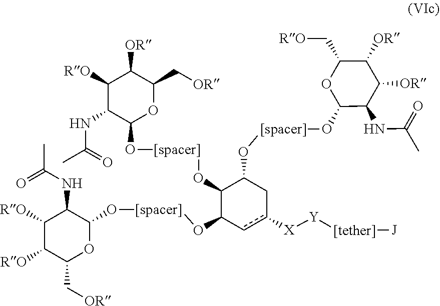

or a pharmaceutically acceptable salt thereof, wherein X, Y, R″, spacer, tether and J are as defined herein, and [symbol] represents a carbon-carbon single bond.

In embodiments, the compound is a compound having the structure of Formula (VId):

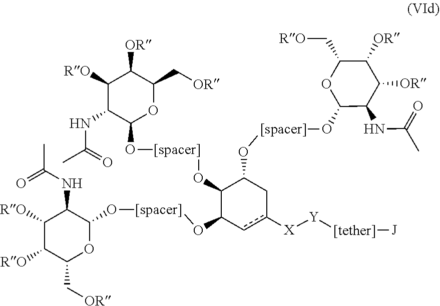

or a pharmaceutically acceptable salt thereof, wherein X, Y, R″, spacer, tether and J are as defined herein, and [symbol] represents a carbon-carbon double bond.

In another aspect, the disclosure provides a compound having the structure of Formula (VII):

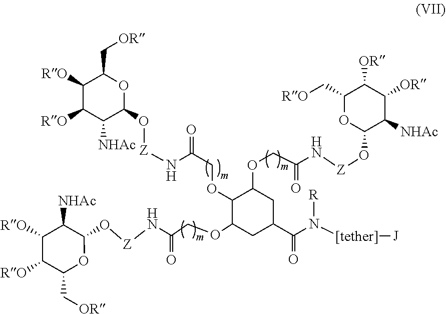

or a pharmaceutically acceptable salt thereof, wherein Z, R, R″, m, tether and J are as defined herein.

In embodiments, the compound is a compound having the structure of Formula (VIIa):

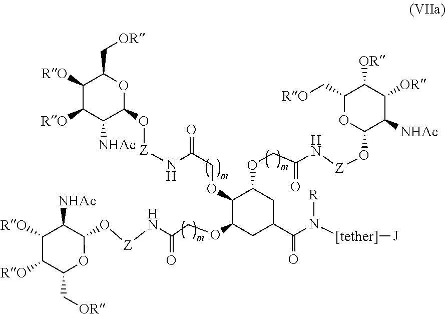

or a pharmaceutically acceptable salt thereof, wherein Z, R, R″, m, tether and J are as defined herein.

In embodiments, the compound is a compound having the structure of Formula (VIIb):

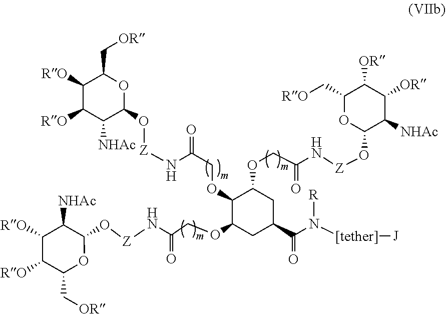

or a pharmaceutically acceptable salt thereof, wherein Z, R, R″, m, tether and J are as defined herein.

In embodiments, the compound is a compound having the structure of Formula (VIIc):

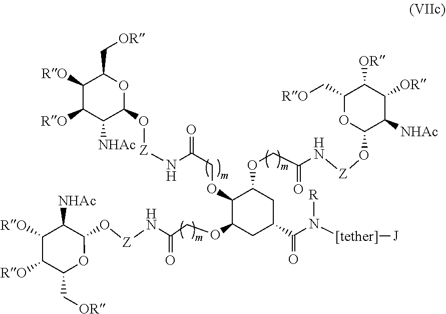

or a pharmaceutically acceptable salt thereof, wherein Z, R, R″, m, tether and J are as defined herein.

In another aspect, the disclosure provides a compound having the structure of Formula (VIII):

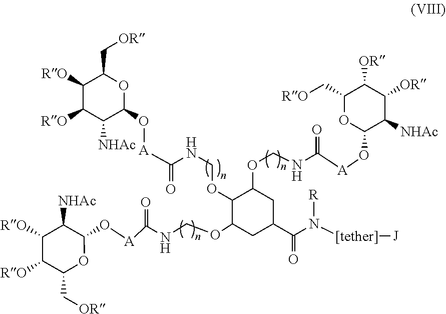

or a pharmaceutically acceptable salt thereof, wherein A, R, R″, n, tether and J are as defined herein.

In embodiments, the compound is a compound having the structure of Formula (VIIIa):

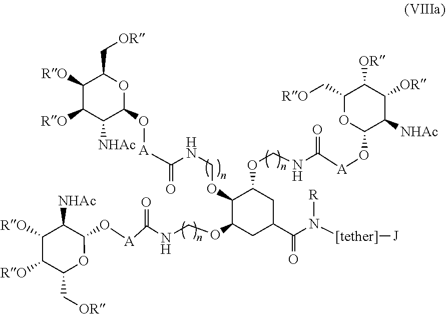

or a pharmaceutically acceptable salt thereof, wherein A, R, R″, n, tether and J are as defined herein.

In embodiments, the compound is a compound having the structure of Formula (VIIIb):

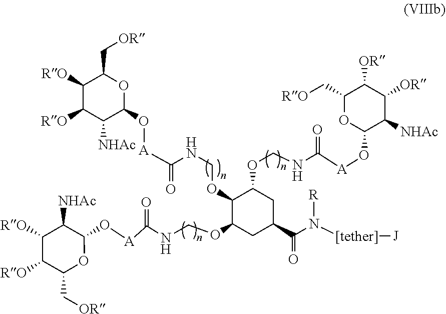

or a pharmaceutically acceptable salt thereof, wherein A, R, R″, n, tether and J are as defined herein.

In embodiments, the compound is a compound having the structure of Formula (VIIIc):

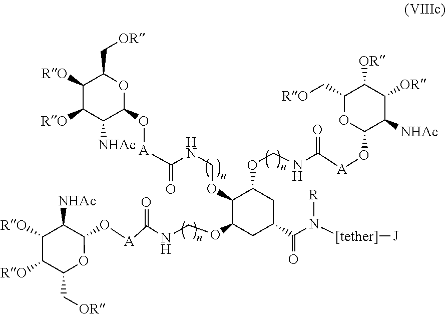

or a pharmaceutically acceptable salt thereof, wherein A, R, R″, n, tether and J are as defined herein.

In another aspect, the disclosure provides a compound having the structure of Formula (X):

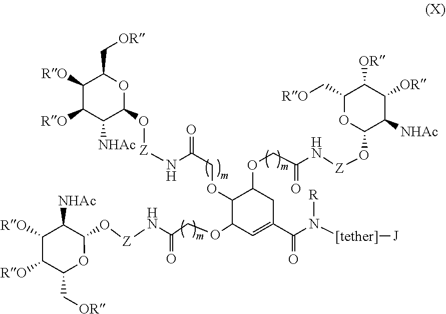

or a pharmaceutically acceptable salt thereof, wherein Z, R, R″, m, tether and J are as defined herein.

In embodiments, the compound is a compound having the structure of Formula (Xa):

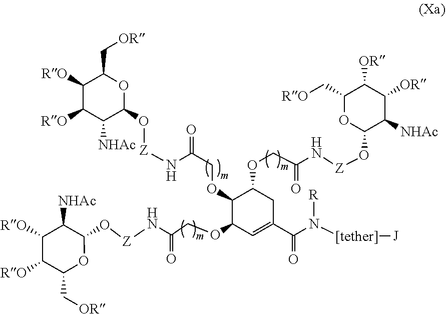

or a pharmaceutically acceptable salt thereof, wherein Z, R, R″, m, tether and J are as defined herein.

In another aspect, the disclosure provides a compound having the structure of Formula (XII):

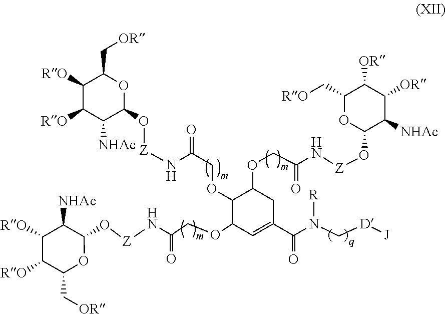

or a pharmaceutically acceptable salt thereof, wherein Z, R, R″, m, q and J are as defined herein, and wherein D′ is selected from (i) a bond, and (ii) —NHC(O)—(CH₂)ₜ— (wherein t is as described herein). In embodiments, D′ is a bond and J is —CO₂H or —C(O)O-(pentafluorophenyl). In other embodiments, D′ is —NHC(O)—(CH₂)ₜ— (e.g., wherein t is 6) and J is —CO₂H or —C(O)O-(pentafluorophenyl).

In embodiments, the compound is a compound having the structure of Formula (XIIa):

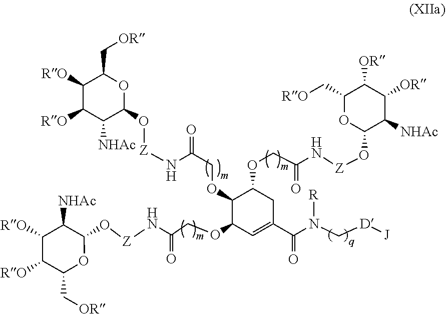

or a pharmaceutically acceptable salt thereof, wherein Z, R, R″, m, q, D′ and J are as defined herein.

In another aspect, the disclosure provides a compound having the structure of Formula (XIV):

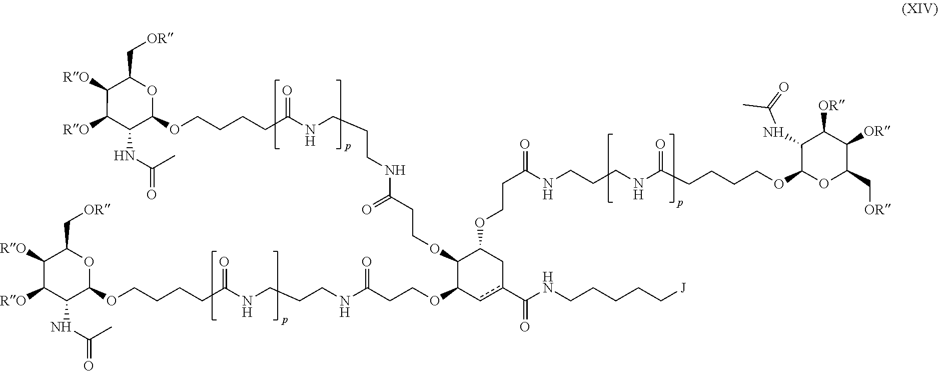

or a pharmaceutically acceptable salt thereof, wherein R″, p and J are as defined herein, and [symbol] represents a carbon-carbon single bond or double bond. In embodiments, R″ is acyl. In embodiments, J is —CO₂H, —OH, or —C(O)O-(pentafluorophenyl). In embodiments, R″ is acyl, and J is —C(O)O-(pentafluorophenyl).

In a related aspect is provided a process for the preparation of a compound of Formula (III), or a pharmaceutically acceptable salt thereof, characterised in that a compound of Formula (VI) is reacted with a compound having the structure

Q-[cargo]

wherein cargo is as defined herein, and Q denotes a group which is reactive to group J as defined herein (e.g., wherein Q denotes a BCN-containing group). In embodiments, the compound of Formula (III) is a compound of Formula (IIIa) and the compound of Formula (VI) is a compound of Formula (VIa). In embodiments, the compound of Formula (III) is a compound of Formula (IIIb) and the compound of Formula (VI) is a compound of Formula (VIb). In embodiments, the compound of Formula (III) is a compound of Formula (IIIc) and the compound of Formula (VI) is a compound of Formula (VIc). In embodiments, the compound of Formula (III) is a compound of Formula (IIId) and the compound of Formula (VI) is a compound of Formula (VId).

In another related aspect is provided a process for the preparation of a compound of Formula (IV), or a pharmaceutically acceptable salt thereof, characterised in that a compound of Formula (VII) is reacted with a compound having the structure

Q-[cargo]

wherein cargo and Q are as defined herein. In embodiments, the compound of Formula (IV) is a compound of Formula (IVa) and the compound of Formula (VII) is a compound of Formula (VIIa). In embodiments, the compound of Formula (IV) is a compound of Formula (IVb) and the compound of Formula (VII) is a compound of Formula (VIIb). In embodiments, the compound of Formula (IV) is a compound of Formula (IVc) and the compound of Formula (VII) is a compound of Formula (VIIc).

In another related aspect is provided a process for the preparation of a compound of Formula (V), or a pharmaceutically acceptable salt thereof, characterised in that a compound of Formula (VIII) is reacted with a compound having the structure

Q-[cargo]

wherein cargo and Q are as defined herein. In embodiments, the compound of Formula (V) is a compound of Formula (Va) and the compound of Formula (VIII) is a compound of Formula (VIIIa). In embodiments, the compound of Formula (V) is a compound of Formula (Vb) and the compound of Formula (VIII) is a compound of Formula (VIIIb). In embodiments, the compound of Formula (V) is a compound of Formula (Vc) and the compound of Formula (VIII) is a compound of Formula (VIIIc).

In another related aspect is provided a process for the preparation of a compound of Formula (IX), or a pharmaceutically acceptable salt thereof, characterised in that a compound of Formula (X) is reacted with a compound having the structure

Q-[cargo]

wherein cargo and Q are as defined herein. In embodiments, the compound of Formula (IX) is a compound of Formula (IXa) and the compound of Formula (X) is a compound of Formula (Xa).

In another related aspect is provided a process for the preparation of a compound of Formula (XI), or a pharmaceutically acceptable salt thereof, characterised in that a compound of Formula (XII) is reacted with a compound having the structure

Q-[cargo]

wherein cargo and Q are as defined herein. In embodiments, the compound of Formula (XI) is a compound of Formula (XIa) and the compound of Formula (XII) is a compound of Formula (XIIa).

In another related aspect is provided a process for the preparation of a compound of Formula (XIII), or a pharmaceutically acceptable salt thereof, characterised in that a compound of Formula (XIV) is reacted with a compound having the structure

Q-[cargo]

wherein cargo and Q are as defined herein.

In embodiments of the above processes, Q denotes a BCN-containing group and J is —N₃. In other embodiments of the above processes, Q denotes an amide-containing group (e.g., a hexyl amine containing group) and J is —CO₂H or —C(O)O-(pentafluorophenyl) (e.g., —C(O)O-(pentafluorophenyl)).

Having been generally described herein, the following non-limiting examples are provided to further illustrate this disclosure.

### Examples

### General Synthetic Schemes

The following scheme, Scheme 1, illustrates an exemplary way of preparing compounds in accordance with the present disclosure and examples:

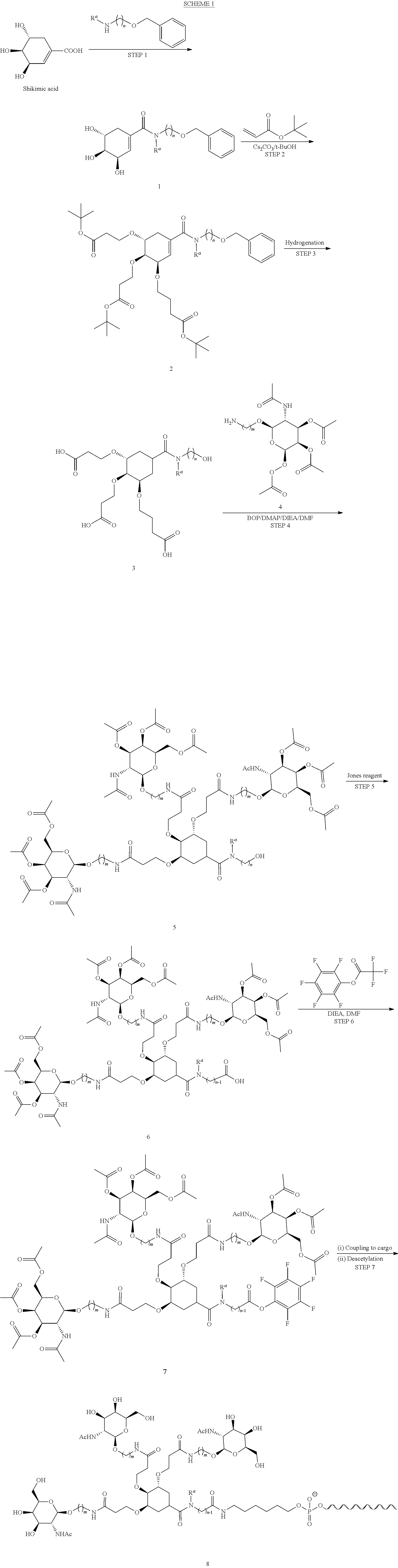

According to Scheme 1, shikimic acid is reacted with an exemplary tether (in which n may be, e.g., 4, 5 or 6, and R^{a} denotes a nitrogen protecting group or N-alkyl group such as, e.g., methyl) in step 1 to form Compound 1. In step 2, Compound 1 is reacted with ^{t}Bu acrylate to form Compound 2. In a third step, the shikimic acid core of Compound 2 is reduced, e.g. using Pd—C/MeOH followed by treatment with HCl, to produce the tri-carboxylic acid Compound 3. In step 4, Compound 3 is reacted with Compound 4 (containing an acetylated GalNac group attached to an alkylamine chain in which m may be, e.g., 4, 5 or 6) to produce Compound 5. In step 5, the free alcohol of the tether group is oxidized to a carboxylic acid which is then reacted with pentafluorophenyl trifluoroacetate in step 6 to yield Compound 7. In step 7, Compound 7 is treated to remove the O-acetyl groups on the GalNAc ligands, and then reacted with a cargo-containing compound (shown with an exemplary hexylamino linker in Scheme 1) followed by treatment with, e.g., aqueous ammonia to form Compound 8.

The following scheme, Scheme 2, illustrates another exemplary way of preparing compounds in accordance with the present disclosure and examples:

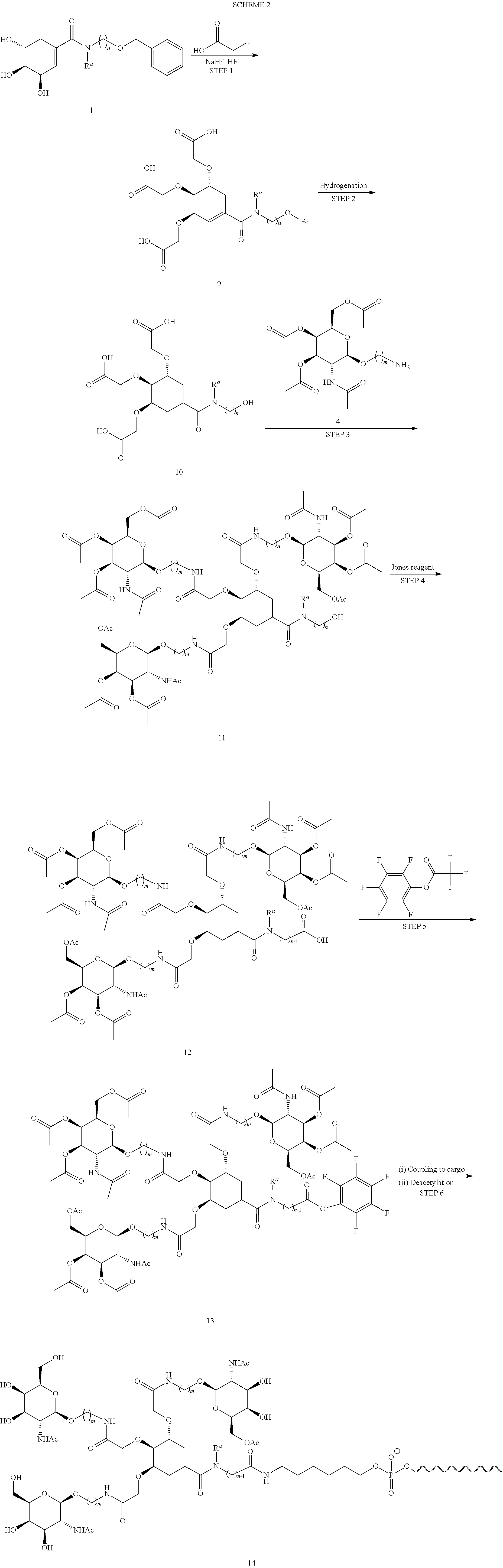

According to Scheme 2, Compound 1 (in which n may be, e.g., 4, 5 or 6, and R^{a} denotes a nitrogen protecting group or N-alkyl group such as, e.g., methyl) is reacted with iodoacetic acid in step 1 to yield Compound 9. In step 2, the shikimic acid core of Compound 9 is reduced, e.g. using Pd—C/MeOH followed by treatment with HCl, to produce the tri-carboxylic acid Compound 10. In step 3, Compound 10 is reacted with Compound 4 (containing an acetylated GalNac group attached to an alkylamine chain in which m may be, e.g., 4, 5 or 6) to produce Compound 11. In step 4, the free alcohol of the tether group is oxidized to a carboxylic acid which is then reacted with pentafluorophenyl trifluoroacetate in step 5 to yield Compound 13. In step 6, Compound 13 is treated to remove the 0-acetyl groups on the GalNAc ligands, and then reacted with a cargo-containing compound (shown with an exemplary hexylamino linker in Scheme 2) followed by treatment with, e.g., aqueous ammonia to form Compound 14.

The following scheme, Scheme 3, illustrates another exemplary way of preparing compounds in accordance with the present disclosure and examples:

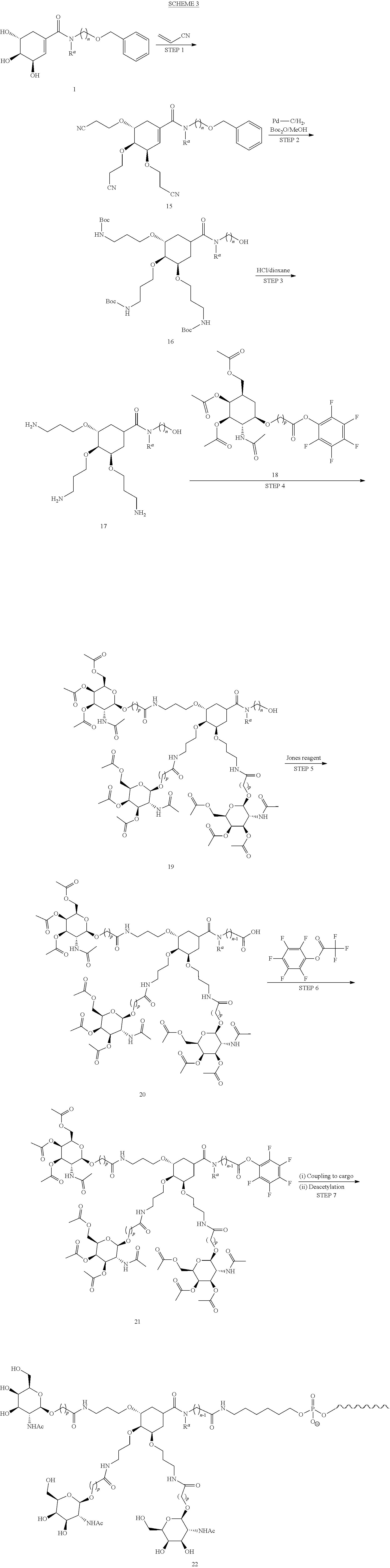

According to Scheme 3, Compound 1 (in which n may be, e.g., 4, 5 or 6, and R^{a} denotes a nitrogen protecting group or N-alkyl group such as, e.g., methyl) is reacted with acrylonitrile in step 1 to yield Compound 15. In step 2, the shikimic acid core of Compound 9 is reduced (illustrated with Pd—C in Scheme 3), and the compound treated with Boc₂O to yield Compound 16. Protecting groups are removed with HCl in step 3 to yield the triamine Compound 17. In step 4, Compound 17 is reacted with Compound 18 (containing an acetylated GalNac group attached to a functionalized alkylester in which p may be, e.g., 3, 4 or 5) to produce Compound 19. In step 5, the free alcohol is oxidized to a carboxylic acid (Compound 20) which is then reacted with pentafluorophenyl trifluoroacetate in step 6 to yield Compound 21. In step 7, Compound 21 is treated to remove the O-acetyl groups on the GalNAc ligands, and then reacted with a cargo-containing compound (shown with an exemplary hexylamino linker in Scheme 3) followed by treatment with, e.g., aqueous ammonia to form Compound 22.

An alternative conjugation method for attaching the cargo-containing moiety to the rest of the molecule is shown in Scheme 4 below:

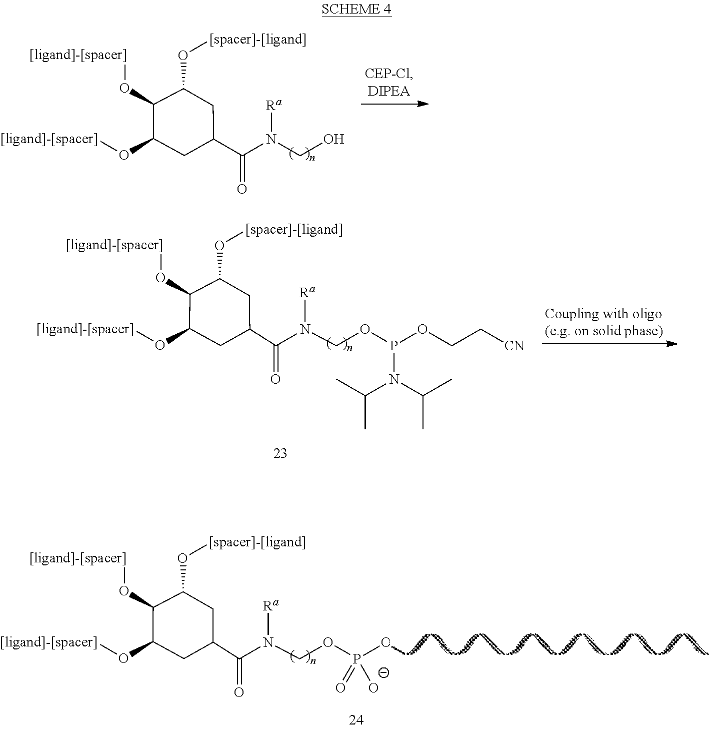

Scheme 4 illustrates, in a general way, how a ligand-containing precursor (e.g., Compound 5, Compound 11, or Compound 19) can be reacted with an agent such as, e.g., 2-cyanoethyl N,N-diisopropylchlorophosphoramidite (CEP-Cl) to yield a phosphoramidite derivative which can be coupled with nucleotides or oligonucleotides to form a compound of the present disclosure. The coupling step can be carried out, for example on solid phase.

In the above general schemes, no specific stereochemistry is indicated for the reduced shikimate ring at the position which is alpha to the amide, e.g. following the step of catalytic hydrogenation. In the synthetic protocols which follow, that position is shown in a configuration having the amide on the same side of the cyclohexane ring as the oxygen atom which is para to that group (i.e., those substituents being in a cis-1,4 relationship). The step of catalytic hydrogenation in the protocols described herein typically produces a single stereoisomer (as assessed by, e.g., HPLC) which is expected to be the cis isomer based on steric considerations. Other synthetic methods may be used to prepare the trans isomer.

### Experimental Techniques

¹H NMR Spectra at 400 and 500 MHz were performed on a Bruker Avance DRX-400 and Bruker Avance DPX-500 spectrometer, respectively, with the chemical shifts (6 in ppm) in the solvent dimethyl sulfoxide-d₆ (DMSO-d₆) referenced at 2.5 ppm at the quoted temperatures. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210<1<400 nm and column Acquity UPLC CSH C18 1.7 μm, dimension 2.1×30 mm, mobile phase H₂O+0.1% HCO₂H/CH₃CN+0.1% HCO₂H.

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich).

### Synthesis of Precursors and Cargo Molecules

### Oligonucleotide Synthesis

Oligonucleotides were synthesized on a 10 μmol scale on a K&A system using CUTAG CPG support (Sigma-Aldrich, 25-35 mol/g). Oligonucleotides were also synthesised on an AKTA OligoPilot Plus 10 synthesizer (Cytiva), on a 32 μmol scale, using a standard synthesis protocol. Nucleotide phosphoramidites were purchased from Sigma-Aldrich or WuXi. Linker phosphoramidites were purchased from Glen Research or WuXi. All cEt phosphoramidites were obtained from Pharmaron. The 5′-amino-modifier C6 was obtained from GlenResearch. Polystyrene solid support (Primer support 5G) was purchased from Cytiva. UV purities were determined using ion-pairing LCMS and are stated at 260 nm. Yields are given based on the initial resin loading and oligonucleotide content of the final product, as calculated from UV absorption.

The sequences of the synthesized oligonucleotides representing an ASO (MALAT1) cargo or an siRNA (ANGPTL3) cargo are shown in the table below:

| | |
|---|---|
| ASO | 5′-GmCATTmCTAATAGmCAGmC-3′ |
| (MALAT1) | (SEQ ID NO: 1) |
| | (5′ PO to HA, all PS backbone) |
| siRNA | Passenger strand: |
| (ANGPTL3) | HA-invAb*gcucaacaUAUuuga |
| | ucagua*invAb (SEQ ID NO: 2) |
| | Guide strand: u* A*c*UgAuCaA |
| | aUaUgUuGaG*c (SEQ ID NO: 3) |

mC denotes 5′-methylcytidine
HA denotes hexylamine
InvAb denotes inverted abasic
* denotes a phosphorothioate linkage
CAPS denote 2′-F in SEQ ID NOs: 2 and 3, and denotes DNA in SEQ ID NO: 1
BOLD denotes 2′-O,4′-C-methylene locked nucleic acid
lower case denotes 2′-OMe

The sequences of the synthesized oligonucleotides representing a siRNA (PPIB) cargo are shown in the table below:

| | |
|---|---|
| siRNA | Passenger strand (18 Mer): |
| (PPIB) | mC*fA*mGfCmAfAmAfUmUfCmCf |
| | AmUfCmGfU*mG*fA (SEQ ID NO: 4) |
| | Passenger strand (20 Mer): |
| | mA*fA*mCfAmGfCmAfAmAfUmUf |
| | CmCfAmUfCmGfU*mG*fA |
| | (SEQ ID NO: 5) |
| | Guide strand (20 Mer): |
| | P-mU*fC*mAfCmGfAmUfGmGfAmAf |
| | UmUfUmGfCmUfG*mU*fU (SEQ ID NO: 6) |

P denotes phosphate
f denotes 2′-fluoro
m denotes 2′-OMe (mC denotes 2′-O-methylcytidine in SEQ ID NOs: 4-6)
* denotes a phosphorothioate linkage

### General Synthetic Procedure

This procedure was used unless otherwise indicated.

Phosphoramidites were dissolved to a final concentration to 0.1 M (3 equivalents) in DNA-grade acetonitrile (ACN) prior to use. Detritylation was performed using 3 vol-% dichloroacetic acid in DCM (contact time 5×35 s). Di- and tri-antennary linkers were deprotected using double detritylation. Activator 42 was used as activating agent (0.25 M in ACN) for the couplings. Recirculation times of phosphoramidites were 4 min for DNA building blocks, 10 min for all 2′-modified building blocks. Triple 10 min coupling was used for linker phosphoramidites and quadruple coupling for GalNAc phosphoramidite. Xanthane hydride was dissolved in pyridine (0.2 M) and used as thiolation reagent with a contact time of 5 min. Oxidizer solution was purchased from Sigma-Aldrich and used as such with a contact time of 9 s. Equal volumes of Cap A (9.1 vol-% acetic anhydride in tetrahydrofuran (THF)) and Cap B (THF/N-methylimidazole/pyridine 80:10:10 vol-%) were mixed in situ for capping (contact time 50 s). Cyanoethyl backbone removal was performed with 20 vol % diethylamine in ACN (contact time 7×1 min) after a final 5′-detritylation where required.

Oligonucleotides were cleaved from the solid support and further deprotected by treatment with methanolic ammonia (3 M) at 55° C. for 15-20 h and were subsequently purified using ion-pairing HPLC on reverse phase columns. For siRNA compounds, single strands were separately prepared as mQ water solutions of equal concentrations, mixed in equal volumes, warmed to 95° C. for 5 min and then allowed to cool down to room temperature over 1 h.

Oligonucleotide was synthesized following general procedure with the exception that 5′-amino-modifier C6 was introduced as last coupling (0.2M, 10 min contact time, double coupling) as last step. The oligonucleotide was purified with the 5′-monomethoxytrityl (MMT) protecting group on. After removal of the MMT group in aqueous acetic acid solution (pH 4.5), the free 5′-NH₂ was reacted with the pentafluorophenyl ester of the GalNAc moiety. Three equivalents of ester were dissolved in ACN and added to a solution of the oligonucleotide in borate buffer (pH 9). Final product was subsequently purified using normal phase HPLC.

For siRNA synthesis, passenger strand (PO to HA) was synthesized using standard oligonucleotide synthesis procedure. Guide strand was synthesized following general synthetic protocol. For annealing, passenger and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. Product solution was adjusted to 1 mM in PBS buffer.

For preparing complexes containing siRNA, passenger strand containing 5′-hexyl amine after MMT deprotection was reacted with the pentafluorophenyl ester (PFP-ester) of the GalNAc moiety. PFP-ester were dissolved in ACN/THF/DMF and added to a solution of the oligonucleotide containing hexyl amine in borate buffer (pH 9). Conjugate products were subsequently purified using HPLC. Once passenger strand containing GalNAc conjugate and guide strands were synthesized, standard annealing procedure was followed for siRNA synthesis. Strand solutions in PBS buffer were mixed and heated for 5 min at 95° C., then slowly cooled down to 20° C. (1h) with gentle shaking. The QC gel was performed, concentrations were adjusted and reannealed if needed. Compounds were stored as 1 mm PBS buffer solutions.

Synthesis of BCN modified hexylamine MALAT1 ASO:

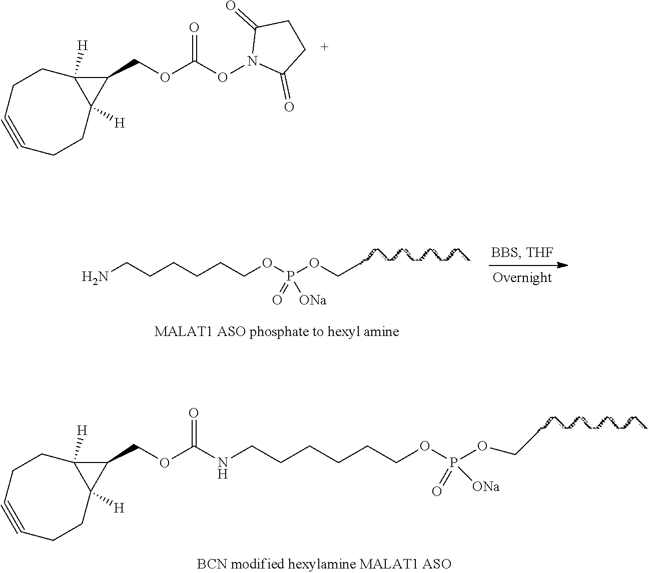

MALAT1-hexylamine modified ASO (50 mg) (all PS, PO to hexyl amine) was first dissolved in 500 μL BBS solvent. NHS-activated BCN (1.5 eq, 4 mg) was dissolved separately in 200 μL THF and added to MALAT1 solution. Reaction mixture became turbid. Reaction mixture was sonicated for couple of minutes and kept under stirring. LCMS showed conversion after 2h. Continued stirring for overnight. Oligonucleotide was precipitated by adding 2 mL of Absolute EtOH and 0.2 mL 3M Sodium Acetate (9:1 V/V). The white precipitate was centrifuged for 15 minutes at 4° C. Removed supernatant layer and repeated washing once more time by 2 mL ethanol. The precipitate after solubilization in 5 mL water was lyophilized overnight to furnish white solid. The obtained product was dissolved in 1 mL deionized water and directly used in the next step. LCMS m/z ES⁻ Expected 5681.704, observed 1894.9 (z=3), 1420.7 (z=4), 1136.34 (z=5).

### Synthesis of Precursor (1)

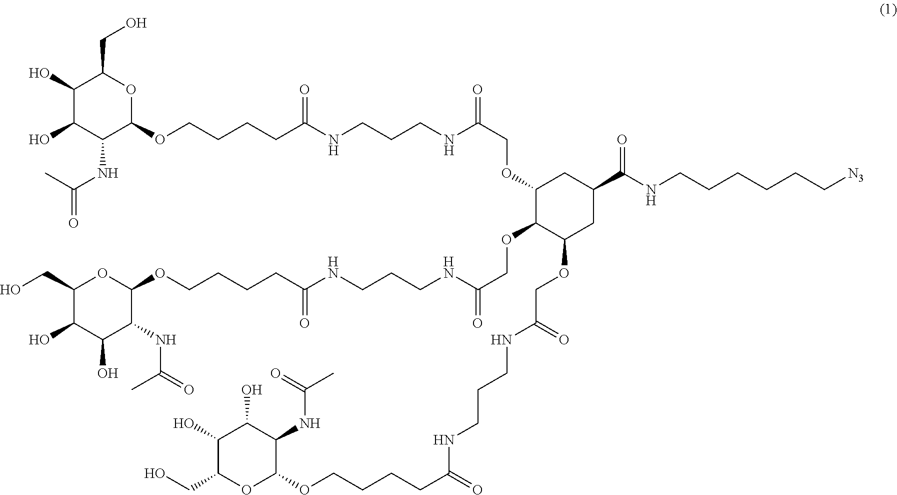 [SMILES: CC(=O)N[C@H]1[C@H](OCCCCC(=O)NCCCNC(=O)CO[C@@H]2C[C@H](C(=O)NCCCCCCN=[N+]=[N-])C[C@@H](OCC(=O)NCCCNC(=O)CCCCO[C@@H]3O[C@H](CO)[C@H](O)[C@H](O)[C@H]3NC(C)=O)[C@@H]2OCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

### Synthetic Scheme:

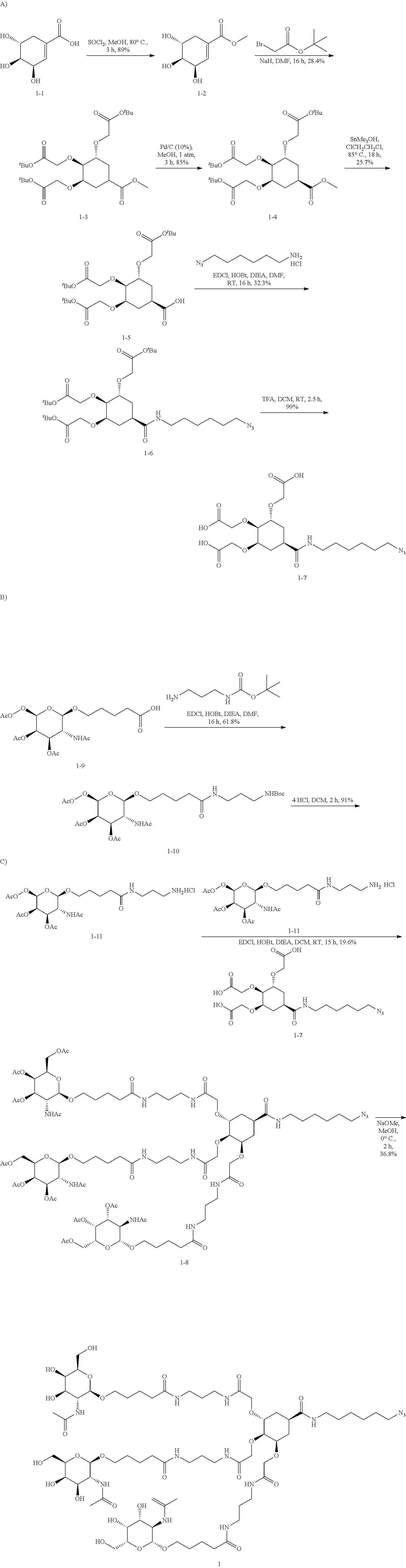

In part (A), SOCl₂ (10.48 ml, 143.55 mmol) was added dropwise to (3R,4S,5R)-3,4,5-trihydroxycyclohex-1-ene-1-carboxylic acid (1-1) (50 g, 287.11 mmol) in MeOH (1000 ml) at 0° C. under argon. The resulting mixture was stirred at 60° C. for 3 hours. The solvent was removed under reduced pressure to afford the crude product. The crude solid was triturated with EtOAc (500 ml)/hexane (500 ml), stirred for 2 h, to give a solid which was collected by filtration and dried under vacuum to give methyl (3R,4S,5R)-3,4,5-trihydroxycyclohex-1-ene-1-carboxylate (1-2) (48.0 g, 89%) as a white solid.

1H-NMR (300 MHz, DMSO, 20° C.) δ 1.99-2.13 (m, 1H), 2.39 (dt, J=4.7, 2.5 Hz, 1H), 2.42-2.49 (m, 1H), 2.51 (q, J=1.9 Hz, 1H), 3.5-3.62 (m, 2H), 3.67 (s, 3H), 3.86 (dt, J=5.8, 4.1 Hz, 1H), 4.18-4.26 (m, 1H), 6.62 (d, J=2.7 Hz, 1H). LCMS m/z ESI Expected 188.0, Observed 187.1 ([M−H]—).

Sodium hydride (7.01 g, 175.36 mmol) was added to (1-2) (10 g, 53.14 mmol) in DMF (100 ml) at 0° C. After stirring for 45 min, tert-butyl 2-bromoacetate (124 g, 635.71 mmol) was added to the mixture reaction. The reaction mixture was warmed to RT and stirred for 16 h.

The reaction mixture was quenched with ice water (100 ml), extracted with EtOAc (200 ml), and washed sequentially with saturated brine (2×100 ml). The organic layer was dried over Na2SO4, filtered and evaporated to afford crude product. The crude product was purified by flash silica chromatography, elution gradient 0 to 40% EtOAc in petroleum ether. Pure fractions were evaporated to dryness to afford tri-tert-butyl 2,2′,2″-(((1R,2S,3R)-5-(methoxycarbonyl)cyclohex-4-ene-1,2,3-triyl)tris(oxy))triacetate (1-3) (8.00 g, 28.4%) as a colourless oil.

1H-NMR (400 MHz, DMSO, 27° C.) δ 1.43 (d, J=5.8 Hz, 27H), 2.16-2.33 (m, 1H), 2.52-2.61 (m, 1H), 3.70 (s, 3H), 3.82-3.94 (m, 2H), 4-4.12 (m, 3H), 4.13-4.28 (m, 4H), 6.77 (d, J=2.8 Hz, 1H). LCMS m/z ESI Expected 530.2, Observed 548.2 ([M+NH₄]⁺).

(1-3) (8 g, 15.08 mmol) and Pd—C(10%) (1.604 g, 1.51 mmol) in MeOH (90 ml) was stirred under an atmosphere of hydrogen at 1 atm and RT for 3 hours. The mixture was filtered through a Celite pad. The solvent was removed under reduced pressure to afford tri-tert-butyl 2,2′,2″-(((1R,3R)-5-(methoxycarbonyl)cyclohexane-1,2,3-triyl)tris(oxy))triacetate (1-4) (6.80 g, 85%) as a yellow oil. The product was used in the next step directly without further purification. Compound (1-4) was isolated as a single diastereoisomer. The stereocenter alpha to the amide is depicted as (S), on the expectation that hydrogenation occurred on the least hindered face of the alkene.

1H-NMR (400 MHz, DMSO, 27° C.) δ 1.42 (d, J=2.0 Hz, 27H), 1.56-1.99 (m, 4H), 3.17 (d, J=5.2 Hz, 1H), 3.55-3.63 (m, 3H), 3.75-3.89 (m, 3H), 4-4.12 (m, 4H), 4.18 (d, J=5.5 Hz, 2H). LCMS m/z ESI Expected 532.2, Observed 550.2 ([M+NH₄]).

Trimethyl tin hydroxide (11.54 g, 63.83 mmol) was added to (1-4) (6.8 g, 12.77 mmol) in DCE (200 ml) at RT under nitrogen. The resulting mixture was stirred at 85° C. for 16 hours.

The reaction mixture was filtered through celite. The solvent was removed under reduced pressure to afford (3R,5R)-3,4,5-tris(2-(tert-butoxy)-2-oxoethoxy)cyclohexane-1-carboxylic acid (crude). The crude product was purified by flash silica chromatography, elution gradient 10 to 80% EtOAc in petroleum ether. Pure fractions were evaporated to dryness to afford (3R,5R)-3,4,5-tris(2-(tert-butoxy)-2-oxoethoxy)cyclohexane-1-carboxylic acid (1-5) (1.700 g, 25.7%) as a colourless oil.

1H-NMR (300 MHz, DMSO, 22° C.) δ 1.42 (d, J=1.5 Hz, 27H), 1.61 (td, J=14.6, 13.6, 9.3 Hz, 2H), 1.86 (dd, J=24.3, 13.2 Hz, 2H), 3.17 (d, J=4.7 Hz, 2H), 3.82 (tt, J=6.6, 3.2 Hz, 2H), 4.03 (t, J=3.1 Hz, 3H), 4.10 (dd, J=4.9, 2.1 Hz, 1H), 4.18 (s, 2H), 12.21 (brs, 1H). LCMS m/z ESI Expected 518.2, Observed 536.3 ([M+H₂O]⁺).

DIEA (2.53 ml, 14.46 mmol) was added to (1-5) (1.5 g, 2.89 mmol), 6-azidohexan-1-amine hydrochloride (1.034 g, 5.78 mmol), HOBt (0.886 g, 5.78 mmol) and EDC (1.109 g, 5.78 mmol) in DMF (25 ml) at RT under nitrogen. The resulting mixture was stirred at RT for 16 hours. The reaction was quenched with water (10 ml), then the solvent was removed under reduced pressure. The crude product was purified by flash silica chromatography, elution gradient 0 to 30% EtOAc in petroleum ether. Pure fractions were evaporated to dryness to afford tri-tert-butyl 2,2′,2″-(((1R,3R)-5-((6-azidohexyl)carbamoyl)cyclohexane-1,2,3-triyl)tris(oxy))triacetate (1-6) (0.600 g, 32.3%) as a yellow oil.

1H-NMR (300 MHz, DMSO, 22° C.) δ 1.23-1.34 (m, 6H), 1.43 (d, J=1.2 Hz, 27H), 1.48-1.74 (m, 7H), 3.02 (dd, J=12.2, 5.9 Hz, 2H), 3.31 (s, 4H), 3.76-3.87 (m, 2H), 4.02 (dd, J=9.0, 5.0 Hz, 3H), 4.18 (s, 2H), 7.7-7.82 (m, 1H). LCMS m/z ESI Expected 642.3, Observed 643.3 ([M+H]⁺).

TFA (6.59 ml, 85.56 mmol) was added to (1-6) (550 mg, 0.86 mmol) in DCM (10 ml) at RT under nitrogen. The resulting mixture was stirred at RT for 2.5 hours. The solvent was removed under reduced pressure to afford 2,2′,2″-(((1R,3R)-5-((6-azidohexyl)carbamoyl) cyclohexane-1,2,3-triyl)tris(oxy))triacetic acid (1-7) (400 mg, 99%) as a yellow oil. The product was used in the next step directly without further purification.

1H-NMR (300 MHz, DMSO, 25° C.) δ 1.16-1.44 (m, 10H), 1.52 (p, J=6.9 Hz, 3H), 1.59-1.71 (m, 3H), 2.79 (d, J=5.0 Hz, 1H), 3.00 (q, J=6.4 Hz, 2H), 3.51-3.67 (m, 2H), 3.84 (d, J=9.4 Hz, 2H), 4.07 (dd, J=5.3, 2.6 Hz, 3H), 7.76 (t, J=5.7 Hz, 1H). Three protons have been exchanged. LCMS m/z ESI Expected 474.4, Observed 475.2 ([M+H]⁺).

In part (B), DIEA (5.86 ml, 33.52 mmol) was added to 5-(((2R,3R,4R,5R,6R)-3-acetamido-4,5-diacetoxy-6-(acetoxymethyl)tetrahydro-2H-pyran-2-yl)oxy)pentanoic acid (1-9) (3 g, 6.70 mmol), EDC (2.57 g, 13.41 mmol), HOBt (1.848 g, 12.07 mmol) and tert-butyl (3-aminopropyl)carbamate (1.752 g, 10.06 mmol) in DMF (60 ml) at RT under nitrogen. The resulting mixture was stirred at RT for 16 hours. The reaction mixture was poured into water (250 ml), extracted with EtOAc (300 ml), and washed with saturated brine (3×250 ml). The organic layer was dried over Na2SO4, filtered and evaporated to afford crude product. The crude product was purified by flash silica chromatography, elution gradient 0 to 40% EtOAc in petroleum ether. Pure fractions were evaporated to dryness to afford (2R,3R,4R,5R,6R)-5-acetamido-2-(acetoxymethyl)-6-((5-((3-((tert-butoxycarbonyl)amino)propyl)amino)-5-oxopentyl)oxy)tetrahydro-2H-pyran-3,4-diyl diacetate (1-10) (2.500 g, 61.8%) as a yellow oil.

1H-NMR (300 MHz, DMSO, 26° C.) δ 1.38 (s, 14H), 1.78 (s, 3H), 1.90 (s, 3H), 2.01 (s, 4H), 2.11 (s, 3H), 2.91 (q, J=6.7 Hz, 2H), 3.02 (q, J=6.7 Hz, 2H), 3.36-3.49 (m, 1H), 3.56-3.65 (m, 1H), 3.66-3.79 (m, 1H), 3.88 (q, J=9.3 Hz, 1H), 4.03 (s, 3H), 4.49 (d, J=8.4 Hz, 1H), 4.98 (dd, J=11.2, 3.4 Hz, 1H), 5.22 (d, J=3.4 Hz, 1H), 6.75 (s, 1H), 7.57-8.1 (m, 2H). LCMS m/z ESI Expected 603.3, Observed 604.1 ([M+H]⁺).

4 M hydrochloric acid in dioxane (20.71 ml, 82.83 mmol) was added to (1-10) (2.5 g, 4.14 mmol) in DCM (20 ml) at RT under nitrogen. The resulting mixture was stirred at RT for 2 hours. The solvent was removed under reduced pressure to afford (2R,3R,4R,5R,6R)-5-acetamido-2-(acetoxymethyl)-6-((5-((3-aminopropyl)amino)-5-oxopentyl)oxy)tetrahydro-2H-pyran-3,4-diyl diacetate (1-11) (1.890 g, 91%) as a yellow oil. The product was used in the next step directly without further purification.

1H-NMR (300 MHz, DMSO, 26° C.) δ 1.50 (s, 6H), 1.78 (d, J=2.9 Hz, 3H), 1.90 (s, 3H), 2.00 (d, J=3.5 Hz, 4H), 2.11 (s, 3H), 2.76 (d, J=7.4 Hz, 3H), 3.01-3.15 (m, 3H), 3.40 (dt, J=12.8, 5.9 Hz, 2H), 3.83-3.93 (m, 1H), 3.96-4.06 (m, 3H), 4.52 (d, J=8.5 Hz, 1H), 4.98 (dd, J=11.2, 3.3 Hz, 1H), 5.22 (d, J=3.4 Hz, 1H), 7.64 (s, 3H). LCMS m/z ESI Expected 503.2, Observed 502.2 (ES-, [M−H]⁺).

In part (C), DIEA (1.988 ml, 11.38 mmol) was added to (1-7) (450 mg, 0.95 mmol), (1-11) (1672 mg, 3.32 mmol), EDC (909 mg, 4.74 mmol) and HOBt (654 mg, 4.27 mmol) in DCM (10 ml) at RT under nitrogen. The resulting mixture was stirred at RT for 15 hours. The solvent was removed under reduced pressure to afford the crude product. The crude product was purified by flash silica chromatography, elution gradient 0 to 25% MeOH in DCM. Pure fractions were evaporated to dryness then re-purified by flash silica chromatography, elution gradient 0 to 20% MeOH in DCM. Pure fractions were evaporated to dryness to afford (2R,2′R,3R,3′R,4R,4′R,5R,5′R,6R,6′R)-((((((2,2′-(((1R,2S,3R,5S)-3-(2-((3-(5-(((2S,3S,4S,5S,6S)-3-acetamido-4,5-diacetoxy-6-(acetoxymethyl)tetrahydro-2H-pyran-2-yl)oxy)pentanamido)propyl)amino)-2-oxoethoxy)-5-((6-azidohexyl)carbamoyl)cyclohexane-1,2-diyl)bis(oxy))bis(acetyl))bis(azanediyl))bis(propane-3,1-diyl))bis(azanediyl))bis(5-oxopentane-5,1-diyl))bis(oxy))bis(5-acetamido-2-(acetoxymethyl)tetrahydro-2H-pyran-6,3,4-triyl) tetraacetate (1-8) (360 mg, 19.66%) as a brown oil.

1H-NMR (400 MHz, DMSO, 24° C.) δ 1.19 (t, J=7.3 Hz, 46H), 1.38-1.62 (m, 7H), 1.78 (s, 3H), 1.89 (s, 3H), 2.00 (s, 3H), 2.11 (s, 5H), 3.05 (q, J=7.3 Hz, 36H), 3.25-3.52 (m, 8H), 3.62-4.12 (m, 11H), 4.50 (d, J=8.5 Hz, 1H), 4.97 (dd, J=11.3, 3.5 Hz, 1H), 5.22 (d, J=3.4 Hz, 1H), 7.31 (dddd, J=27.1, 8.1, 6.8, 1.1 Hz, 4H), 7.55 (dt, J=8.3, 1.1 Hz, 2H), 7.8-7.91 (m, 4H). LCMS m/z ESI⁺ Expected 1929.9, Observed 966.3 (z=2).

Sodium methoxide (210 mg, 1.17 mmol) was added to (1-8) (250 mg, 0.13 mmol) in MeOH (10 ml) at 0° C. under nitrogen. The resulting mixture was stirred at RT for 2 hours. AcOH (0.067 ml, 1.17 mmol) was added the reaction mixture. The solvent was removed under reduced pressure to afford the crude product. The crude product was purified by flash C18-flash chromatography, elution gradient 0 to 25% MeCN in water (0.05% TFA). Pure fractions were evaporated to dryness to afford N,N′-(((2,2′-(((1R,2S,3R,5S)-3-(2-((3-(5-(((2S,3S,4S,5S,6S)-3-acetamido-4,5-dihydroxy-6-(hydroxymethyl)tetrahydro-2H-pyran-2-yl)oxy)pentanamido)propyl)amino)-2-oxoethoxy)-5-((6-azidohexyl)carbamoyl)cyclohexane-1,2-diyl)bis(oxy))bis(acetyl))bis(azanediyl))bis(propane-3,1-diyl))bis(5-(((2R,3R,4R,5R,6R)-3-acetamido-4,5-dihydroxy-6-(hydroxymethyl)tetrahydro-2H-pyran-2-yl)oxy)pentanamide) (1) (74.0 mg, 36.8%) as a white solid.

1H-NMR (400 MHz, DMSO, 25° C.) δ 1.21-1.59 (m, 26H), 1.78 (d, J=17.0 Hz, 14H), 1.97-2.18 (m, 6H), 2.26-2.51 (m, 1H), 3.08 (dtd, J=26.7, 13.2, 12.7, 6.7 Hz, 13H), 3.30 (q, J=6.9 Hz, 6H), 3.4-3.45 (m, 3H), 3.53 (ddd, J=13.9, 11.0, 5.6 Hz, 5H), 3.64-3.77 (m, 8H), 3.86 (d, J=14.5 Hz, 2H), 3.94-4.12 (m, 4H), 4.22 (d, J=8.4 Hz, 2H), 4.48-4.77 (m, 8H), 6.68 (s, 1H), 6.98 (dddd, J=17.3, 7.7, 6.6, 1.2 Hz, 4H), 7.41 (d, J=8.1 Hz, 3H), 7.51 (d, J=8.2 Hz, 2H), 7.69 (dd, J=9.3, 3.5 Hz, 2H), 7.76-7.97 (m, 7H). LCMS m/z ESI⁺ Expected 1551.8, Observed 777.0 (z=2).

### Synthesis of Precursor (2)

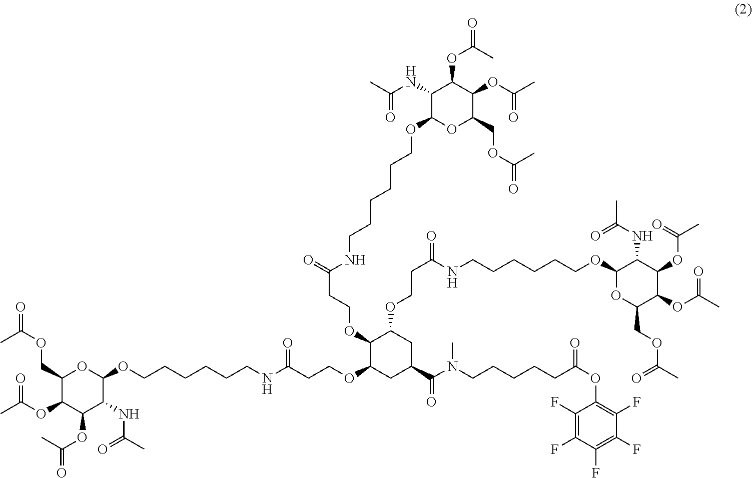 [SMILES: CC(=O)N[C@H]1[C@H](OCCCCCCNC(=O)CCO[C@@H]2C[C@H](C(=O)N(C)CCCCCC(=O)Oc3c(F)c(F)c(F)c(F)c3F)C[C@@H](OCCC(=O)NCCCCCCO[C@@H]3O[C@H](COC(C)=O)[C@H](OC(C)=O)[C@H](OC(C)=O)[C@H]3NC(C)=O)[C@@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

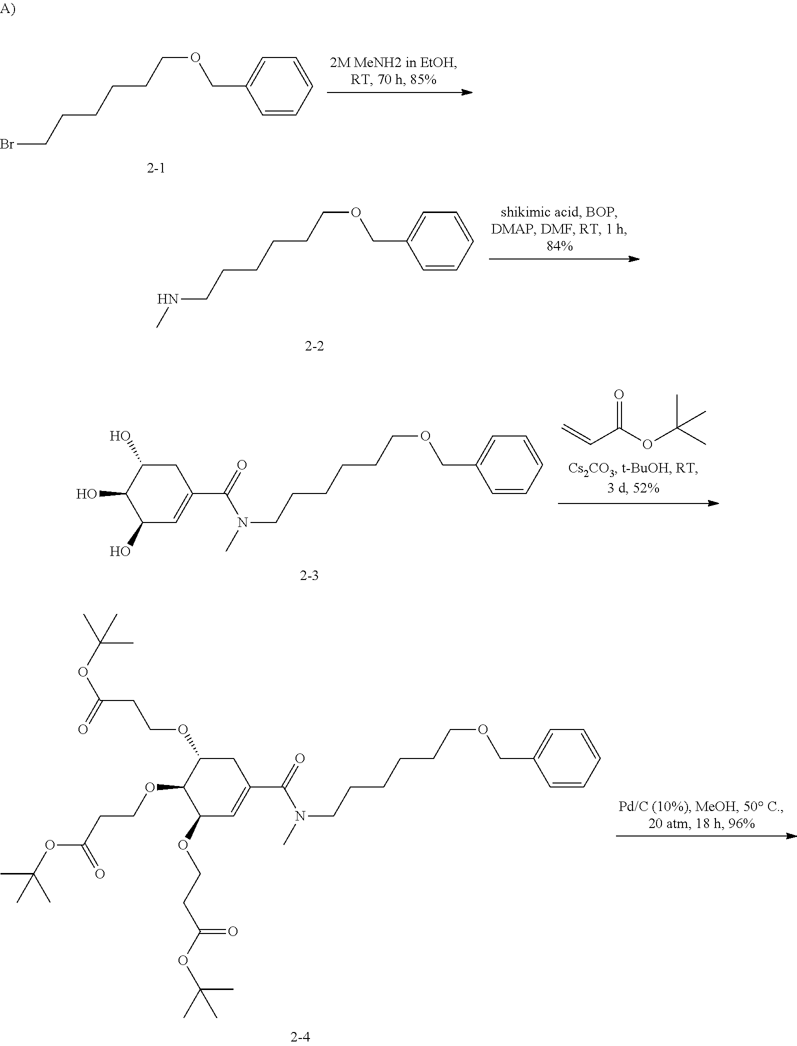

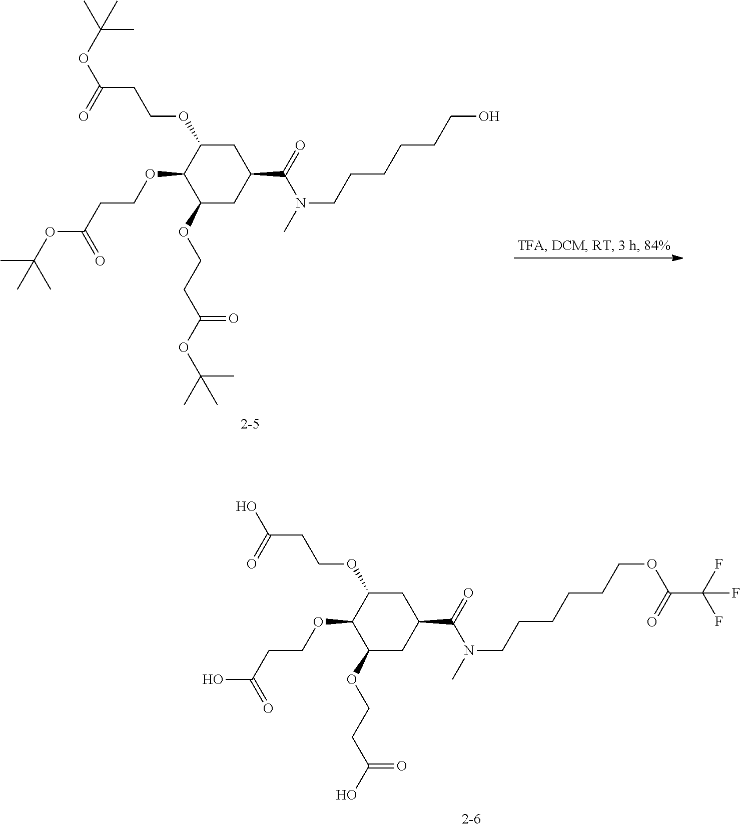 [SMILES: CN(CCCCCCO)C(=O)[C@H]1C[C@@H](OCCC(=O)OC(C)(C)C)[C@@H](OCCC(=O)OC(C)(C)C)[C@H](OCCC(=O)OC(C)(C)C)C1 ; CN(CCCCCCOC(=O)C(F)(F)F)C(=O)[C@H]1C[C@@H](OCCC(=O)O)[C@@H](OCCC(=O)O)[C@H](OCCC(=O)O)C1]

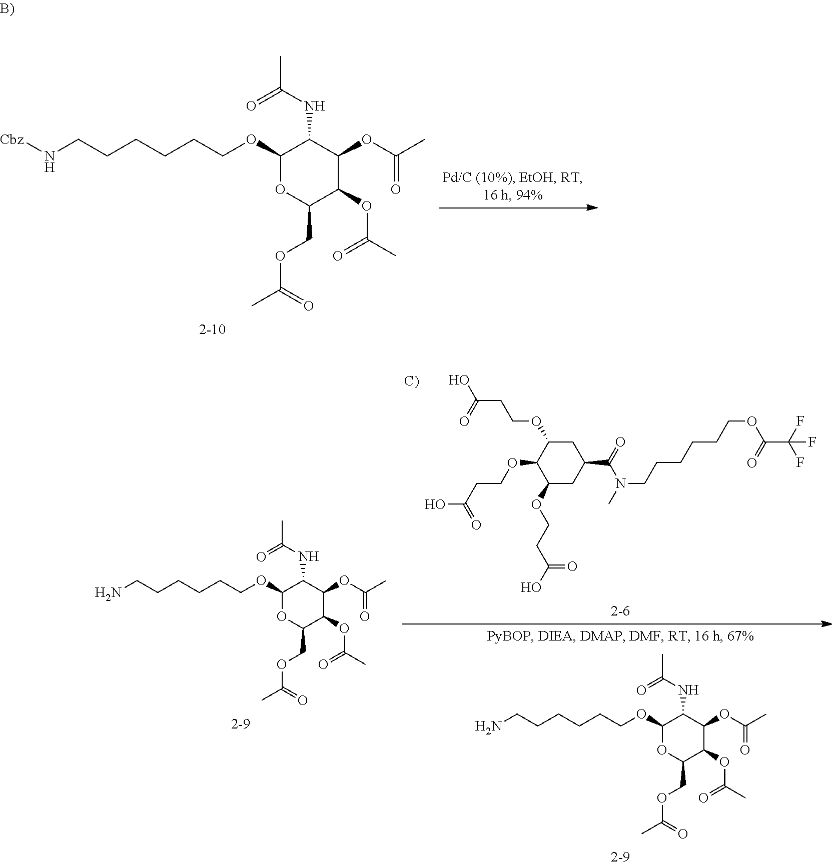 [SMILES: CC(=O)N[C@H]1[C@H](OCCCCCCNC(=O)OCc2ccccc2)O[C@H](COC(C)=O)[C@H](OC(C)=O)[C@@H]1OC(C)=O ; CC(=O)N[C@H]1[C@H](OCCCCCCN)O[C@H](COC(C)=O)[C@H](OC(C)=O)[C@@H]1OC(C)=O ; CN(CCCCCCOC(=O)C(F)(F)F)C(=O)[C@H]1C[C@@H](OCCC(=O)O)[C@@H](OCCC(=O)O)[C@H](OCCC(=O)O)C1 ; CC(=O)N[C@H]1[C@H](OCCCCCCN)O[C@H](COC(C)=O)[C@H](OC(C)=O)[C@@H]1OC(C)=O]

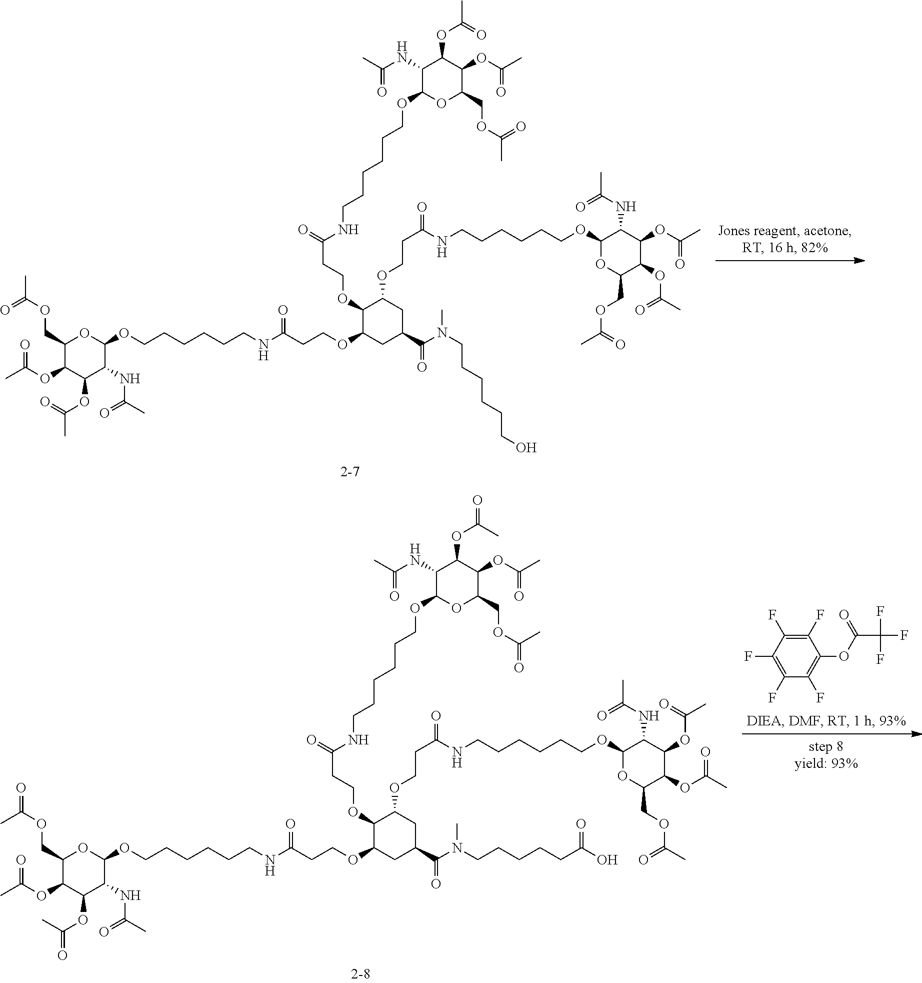

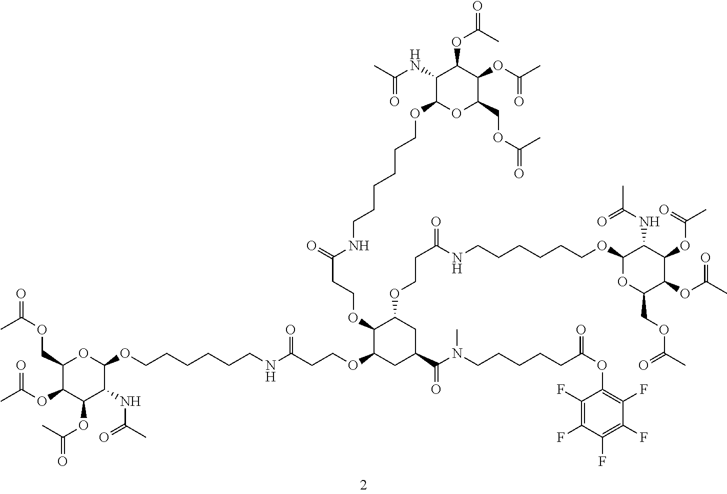 [SMILES: CC(=O)N[C@H]1[C@H](OCCCCCCNC(=O)CCO[C@@H]2C[C@H](C(=O)N(C)CCCCCC(=O)Oc3c(F)c(F)c(F)c(F)c3F)C[C@@H](OCCC(=O)NCCCCCCO[C@@H]3O[C@H](COC(C)=O)[C@H](OC(C)=O)[C@H](OC(C)=O)[C@H]3NC(C)=O)[C@@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

In part (A), a solution of (((6-bromohexyl)oxy)methyl)benzene (2-1) (100 g, 184.37 mmol) in 2M MeNH₂ in ethanol (3000 mL) was stirred at RT for 70 hours. The solvent was removed under reduced pressure to afford 6-(benzyloxy)-N-methylhexan-1-amine (2-2) (70.0 g, 85%) as a white solid. The product was used in the next step directly without further purification.

1H-NMR (500 MHz, DMSO, 24° C.) δ 1.32 (dp, J=11.1, 7.0, 5.6 Hz, 4H), 1.55 (dq, J=13.3, 7.0, 6.5 Hz, 4H), 2.54 (s, 3H), 2.77-2.92 (m, 2H), 3.42 (t, J=6.5 Hz, 2H), 4.45 (s, 2H), 7.18-7.43 (m, 5H), 8.38 (s, 1H). LCMS m/z ESI Expected 221.3, Observed 222.2 ([M+H]⁺).

(2-2) (35 g, 158.12 mmol) was added to N-ethyl-N-isopropylpropan-2-amine (83 mL, 474.37 mmol), shikimic acid (30.3 g, 173.94 mmol), BOP (105 g, 237.19 mmol) and DMAP (19.32 g, 158.12 mmol) in DMF (700 mL). The resulting mixture was stirred at RT for 1 hour. The reaction mixture was poured into water (1 L), extracted with EtOAc (2×1 L), the organic layer was washed with saturated brine (2×1 L), the organic layer was dried over Na2SO4, filtered and evaporated to afford brown gum. The crude product was purified by flash silica chromatography, elution gradient 0 to 20% MeOH in DCM. Pure fractions were evaporated to dryness to afford (3R,4S,5R)—N-(6-(benzyloxy)hexyl)-3,4,5-trihydroxy-N-methylcyclohex-1-ene-1-carboxamide (2-3) (50.0 g, 84%) as a yellow oil.

1H-NMR (500 MHz, DMSO, 26° C.) δ 1.21-1.25 (m, 2H), 1.33 (s, 2H), 1.41-1.56 (m, 4H), 1.88-1.97 (m, 1H), 2.33-2.42 (m, 1H), 2.81 (s, 2H), 2.92 (s, 1H), 3.25 (d, J=26.1 Hz, 3H), 3.41 (t, J=6.5 Hz, 2H), 3.50 (dd, J=7.1, 4.0 Hz, 1H), 3.83 (dt, J=7.1, 5.1 Hz, 1H), 4.13 (s, 1H), 4.45 (s, 2H), 5.48 (t, J=2.2 Hz, 1H), 6.73-6.74 (m, 1H), 7.25-7.38 (m, 5H), 8.15 (d, J=5.2 Hz, 1H). LCMS m/z ESI Expected 377.2, Observed 378.2 ([M+H]⁺).

Cs₂CO₃ (85 g, 262.26 mmol) was added to (2-3) (30 g, 79.47 mmol) and tert-butyl acrylate (698 mL, 4768.45 mmol) in t-BuOH (1500 mL). The resulting mixture was stirred by mechanical stirring at RT for 3 days. The reaction mixture was diluted with EtOAc (3000 ml). The solutions was filtered through celite. The solvent was removed under reduced pressure to afford the crude product. The crude product was purified by flash silica chromatography, elution gradient 0 to 45% EtOAc in petroleum ether. Pure fractions were evaporated to dryness to afford tri-tert-butyl 3,3′,3″-(((1R,2S,3R)-5-((6-(benzyloxy)hexyl)(methyl)carbamoyl)cyclohex-4-ene-1,2,3-triyl)tris(oxy))tripropionate. (45 g, crude 2, contain di-product). The crude product 2 was purified by flash silica (330 g silicone column) chromatography, elution gradient 0 to 35.5% EtOAc in petroleum ether. Pure fractions were evaporated to dryness to afford tri-tert-butyl 3,3′,3″-(((1R,2S,3R)-5-((6-(benzyloxy)hexyl)(methyl)carbamoyl)cyclohex-4-ene-1,2,3-triyl)tris(oxy))tripropionate (2-4) (31.5 g, 52.0%) as a colourless oil.

1H-NMR (300 MHz, DMSO, 22° C.) δ 1.39 (d, J=1.8 Hz, 34H), 1.99 (s, 2H), 2.40 (dq, J=7.4, 4.5, 3.9 Hz, 7H), 2.83 (d, J=24.1 Hz, 3H), 3.25 (s, 2H), 3.41 (t, J=6.4 Hz, 2H), 3.58-3.83 (m, 8H), 3.97-4.06 (m, 1H), 4.44 (s, 2H), 5.51 (s, 1H), 7.21-7.41 (m, 5H). LCMS m/z ESI Expected 761.4, Observed 762.6 ([M+H]⁺).

Pd/C (10%, 50 w/w % water) (2.79 g, 2.62 mmol) was added to (2-4) in MeOH (200 mL), the mixture was stirred under an atmosphere of hydrogen (20 atm) at 50° C. for 18 hours. The reaction mixture was filtered through the Buchner funnel. The solvent was removed under reduced pressure, the resulting oil was dissolved in acetonitrile then concentrated to dryness to afford tri-tert-butyl 3,3′,3″-(((1R,3R)-5-((6-hydroxyhexyl)(methyl)carbamoyl)cyclohexane-1,2,3-triyl)tris(oxy))tripropionate (2-5) (8.50 g, 96%) as a colourless oil. The product was used in the next step directly without further purification. Compound (2-5) was isolated as a single diastereoisomer. The stereocenter alpha to the amide is depicted as (S), on the expectation that hydrogenation occurred on the least hindered face of the alkene.

1H-NMR (300 MHz, DMSO, 23° C.) δ 1.09-1.29 (m, 4H), 1.34-1.45 (m, 27H), 1.56 (t, J=11.5 Hz, 7H), 2.21-2.47 (m, 6H), 2.75 (d, J=9.4 Hz, 2H), 2.95 (s, 2H), 3.25 (q, J=8.3, 7.2 Hz, 2H), 3.33-3.51 (m, 4H), 3.65 (dt, J=16.2, 9.7 Hz, 6H), 3.79 (dt, J=10.9, 5.6 Hz, 2H), 4.33 (td, J=5.2, 2.2 Hz, 1H). LCMS m/z ESI Expected 673.4, Observed 674.4 ([M+H]⁺).

TFA (22.87 mL, 296.79 mmol) was added to (2-5) (2.0 g, 2.97 mmol) in DCM (20 ml) at RT. The resulting mixture was stirred at RT for 3 hours. The solvent was removed under reduced pressure to afford 3,3′,3″-(((1R,2S,3R,5S)-5-(N-methyl(6-(2,2,2-trifluoroacetoxy)hexyl) carbamoyl)cyclohexane-1,2,3-triyl)tris(oxy))tripropionic acid (2-6) (1.500 g, 84%) as a colourless oil. The product was used in the next step directly without further purification.

1H-NMR (300 MHz, DMSO, 25° C.) δ 1.29-1.39 (m, 4H), 1.54 (d, J=8.8 Hz, 4H), 1.68 (p, J=6.6 Hz, 4H), 2.43 (d, J=6.3 Hz, 6H), 2.95 (s, 3H), 3.23 (t, J=7.4 Hz, 3H), 3.63 (dqt, J=15.5, 9.6, 4.9 Hz, 11H). LCMS m/z ESI Expected 601.2, Observed 602.3 ([M+H]⁺).

In part (B), Pd—C(10%, 50 w/w % water) (0.641 g, 0.60 mmol) was added to (2R,3R,4R,5R,6R)-5-acetamido-2-(acetoxymethyl)-6-((6-(((benzyloxy)carbonyl)amino) hexyl)oxy)tetrahydro-2H-pyran-3,4-diyl diacetate (2-10) (5 g, 8.61 mmol) (12) in EtOH (100 mL) at RT. The resulting solution was stirred under an atmosphere of hydrogen at RT for 16 hours. The reaction mixture was filtered through celite to afford crude product, washed with MeOH (3×150 ml). The solvent was removed under reduced pressure to afford (2R,3R,4R,5R,6R)-5-acetamido-2-(acetoxymethyl)-6-((6-aminohexyl)oxy)tetrahydro-2H-pyran-3,4-diyl diacetate (2-9) (3.60 g, 94%) as a colourless oil, which was used without further purification.

1H-NMR (300 MHz, DMSO, 24° C.) δ 1.29 (d, J=16.4 Hz, 6H), 1.46 (s, 2H), 1.78 (d, J=1.2 Hz, 3H), 1.90 (s, 3H), 2.00 (s, 3H), 2.09 (d, J=9.1 Hz, 8H), 3.34-3.49 (m, 1H), 3.70 (dt, J=9.7, 6.1 Hz, 1H), 3.78-3.93 (m, 1H), 4.03 (s, 2H), 4.49 (d, J=8.5 Hz, 1H), 4.97 (dd, J=11.2, 3.4 Hz, 1H), 5.22 (d, J=3.4 Hz, 1H), 7.83 (d, J=9.2 Hz, 1H). LCMS m/z ESI Expected 446.2, Observed 447.2 ([M+H]⁺).

(2-6) (0.5 g, 0.83 mmol) was added to N-ethyl-N-isopropylpropan-2-amine (1.452 mL, 8.31 mmol), PyBOP (1.838 g, 4.16 mmol), (2-9) (1.299 g, 2.91 mmol) and DMAP (0.305 g, 2.49 mmol) in DMF (25 mL). The resulting mixture was stirred at RT for 16 hours. The solvent was removed under reduced pressure to afford the crude product. The crude product was purified by flash C18-flash chromatography, elution gradient 0 to 38% MeCN in water (0.1% FA). Pure fractions were evaporated to dryness to afford a yellow oil. The oil was dissolved in acetonitrile and water then dried by lyophilization to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-[6-hydroxyhexyl(methyl)carbamoyl]cyclohexoxy]propanoylamino]hexoxy]tetrahydropyran-2-yl]methyl acetate (2-7) (1.000 g, 67.2%) as a yellow solid.

1H-NMR (300 MHz, DMSO, 25° C.) δ 1.11-1.31 (m, 17H), 1.32-1.58 (m, 21H), 1.77 (s, 9H), 1.90 (s, 8H), 2.00 (s, 9H), 2.11 (s, 8H), 2.17-2.38 (m, 6H), 2.75 (d, J=10.8 Hz, 3H), 2.97 (d, J=9.9 Hz, 2H), 3-3.13 (m, 5H), 3.22 (d, J=10.4 Hz, 3H), 3.35-3.42 (m, 5H), 3.5-3.78 (m, 12H), 3.87 (dt, J=11.2, 8.8 Hz, 3H), 4.03 (s, 8H), 4.35 (dt, J=15.4, 5.3 Hz, 1H), 4.49 (d, J=8.4 Hz, 3H), 4.97 (dd, J=11.2, 3.4 Hz, 3H), 5.22 (d, J=3.4 Hz, 3H), 7.45-8.24 (m, 6H). LCMS m/z ESI Expected 1789.9, Observed 1790.7 ([M+H]⁺).

Chromosulfuric acid (1.228 mL, 2.46 mmol) was added to (2-7) (2.2 g, 1.23 mmol) in acetone (25 mL) at 0° C. The resulting mixture was stirred at RT for 16 hours. The reaction was quenched with i-PrOH (50 ml), the solvent was removed under reduced pressure. The crude product was diluted by DCM (100 ml), washed sequentially with water (100 mL), saturated NaHCO₃ (2×100 mL), and saturated brine (100 mL). The organic layer was dried over Na2SO4, filtered and evaporated to afford the crude product. The crude product was purified by flash C18-flash chromatography, elution gradient 0 to 40% MeCN in water (0.1% FA). Pure fractions were evaporated to dryness to afford a colourless oil, which was dried by lyophilization to afford 6-((3R,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)-N-methylcyclohexane-1-carboxamido)hexanoic acid (2-8) (2.300 g, 82%) as a white solid.

1H-NMR (500 MHz, DMSO, 25° C.) δ 1.17-1.31 (m, 15H), 1.32-1.35 (m, 7H), 1.37-1.41 (m, 7H), 1.49-1.67 (m, 7H), 1.77 (s, 9H), 1.90 (s, 8H), 2.00 (s, 8H), 2.11 (s, 8H), 2.20 (dt, J=15.0, 7.5 Hz, 2H), 2.25-2.34 (m, 5H), 2.66-2.84 (m, 3H), 2.95 (s, 2H), 2.98-3.14 (m, 6H), 3.17-3.33 (m, 3H), 3.33-3.46 (m, 4H), 3.5-3.59 (m, 5H), 3.6-3.79 (m, 7H), 3.87 (dt, J=11.5, 8.9 Hz, 3H), 4.03 (q, J=4.0 Hz, 9H), 4.49 (dd, J=8.4, 1.4 Hz, 3H), 4.97 (dd, J=11.2, 3.4 Hz, 3H), 5.22 (d, J=3.4 Hz, 3H), 7.6-8.04 (m, 6H). LCMS m/z ESI⁺ Expected 1803.8, Observed 903.4 (z=2).

Perfluorophenyl 2,2,2-trifluoroacetate (0.286 mL, 1.66 mmol) was added to (2-8) (1 g, 0.55 mmol) and DIEA (0.290 mL, 1.66 mmol) in DMF (20 mL) at RT. The resulting mixture was stirred at RT for 1 hour. The reaction mixture was diluted with EtOAc (50 mL), and washed sequentially with 1M NaHSO4 (4×50 mL), saturated NaHCO₃ (2×50 ml), and saturated brine (50 mL). The organic layer was dried over Na2SO4, filtered and evaporated to dryness, then dried by lyophilization to afford (2,3,4,5,6-pentafluorophenyl) 6-[methyl-[(3R,5R)-3,4,5-tris[3-[6-[(2R,3R,4R,5R,6R)-3-acetamido-4,5-diacetoxy-6-(acetoxymethyl)tetrahydropyran-2-yl]oxyhexylamino]-3-oxo-propoxy]cyclohexanecarbonyl]amino]hexanoate (2) (1.02 g, 93%) as a yellow solid.

1H-NMR (300 MHz, DMSO, 22° C.) δ 0.95-1.3 (m, 15H), 1.40 (d, J=27.5 Hz, 19H), 1.62-1.72 (m, 2H), 1.77 (s, 8H), 1.89 (s, 8H), 1.99 (s, 9H), 2.10 (s, 8H), 2.17-2.38 (m, 6H), 2.78 (dt, J=10.2, 3.6 Hz, 4H), 2.88-3.11 (m, 8H), 3.17-3.36 (m, 3H), 3.37-3.46 (m, 5H), 3.49-3.79 (m, 11H), 3.87 (q, J=9.2 Hz, 3H), 4.01 (d, J=3.4 Hz, 8H), 4.49 (d, J=8.4 Hz, 3H), 4.97 (dd, J=11.2, 3.4 Hz, 3H), 5.21 (d, J=3.4 Hz, 3H), 7.65-7.92 (m, 5H), 11.63 (s, 1H). LCMS m/z ESI Expected 1969.8, Observed 1971.2 ([M+H]⁺).

### Synthesis of Precursor (3)

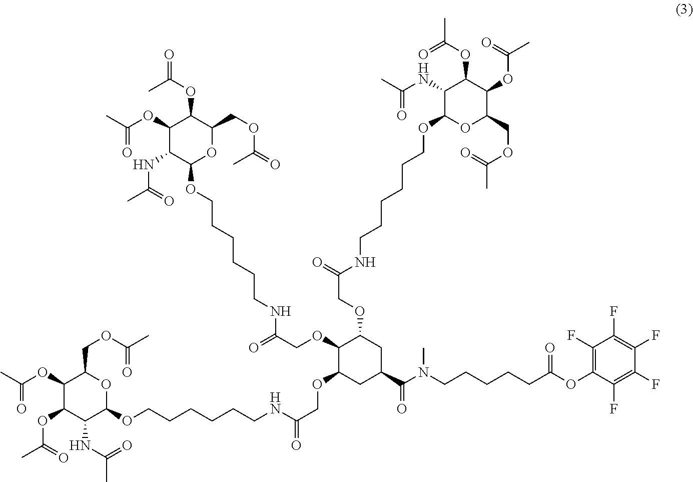 [SMILES: CC(=O)N[C@H]1[C@H](OCCCCCCNC(=O)CO[C@@H]2C[C@H](C(=O)N(C)CCCCCC(=O)Oc3c(F)c(F)c(F)c(F)c3F)C[C@@H](OCC(=O)NCCCCCCO[C@@H]3O[C@H](COC(C)=O)[C@H](OC(C)=O)[C@H](OC(C)=O)[C@H]3NC(C)=O)[C@@H]2OCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

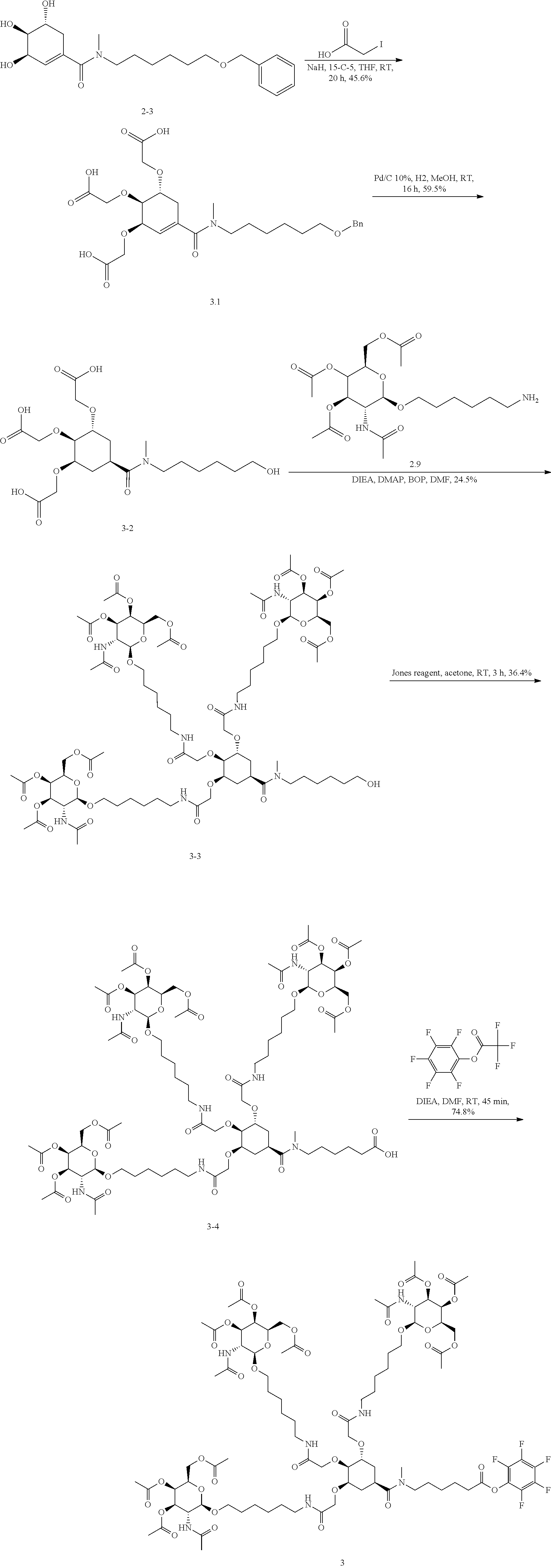

Sodium hydride (2.96 g, 73.91 mmol) was added to (3R,4S,5R)—N-(6-(benzyloxy)hexyl)-3,4,5-trihydroxy-N-methylcyclohex-1-ene-1-carboxamide (2-3) (3 g, 7.95 mmol) in THF (100 mL) at 0° C. under nitrogen. The mixture was stirred at 0° C. for 2 hours, 15-Crown-5 (0.875 g, 3.97 mmol) and 2-iodoacetic acid (8.87 g, 47.68 mmol) were added to the mixture. The resulting mixture was stirred at RT for 20 hours. The reaction was quenched with saturated NH₄Cl (15 ml), then the solvent was removed under reduced. The crude product was purified by flash C18-flash chromatography, elution gradient 0 to 50% MeCN in water (0.05% TFA). Pure fractions were evaporated to dryness to afford 2,2′,2″-(((1R,2S,3R)-5-((6-(benzyloxy)hexyl)(methyl)carbamoyl)cyclohex-4-ene-1,2,3-triyl)tris(oxy))triacetic acid (3-1) (2.000 g, 45.6%) as a yellow gum.

1H-NMR (400 MHz, DMSO, 23° C.) δ 1.12-1.39 (m, 4H), 1.54 (d, J=12.8 Hz, 4H), 2.21 (ddd, J=40.1, 16.3, 8.7 Hz, 1H), 2.86 (d, J=43.7 Hz, 3H), 3.42 (s, 5H), 3.82 (d, J=19.2 Hz, 3H), 4.03-4.39 (m, 6H), 4.44 (s, 2H), 5.80 (d, J=71.3 Hz, 1H), 7.31 (dp, J=19.2, 7.2, 6.8 Hz, 5H). Three protons has been exchanged. LCMS m/z ESI Expected 551.2, Observed 552.2 ([M+H]⁺).

Pd/C (10%) (1.929 g, 1.81 mmol) was added to (3-1) (1 g, 1.81 mmol) in MeOH (25 mL). The resulting mixture was stirred at rt for 16 hours under hydrogen. The reaction mixture was filtered through celite, and washed with methanol (200 ml). The solvent was removed under reduced pressure, dried under vacuum to afford 2,2′,2″-(((1R,2S,3R)-5-((6-hydroxyhexyl) (methyl)carbamoyl)cyclohexane-1,2,3-triyl)tris(oxy))triacetic acid (3-2) (0.500 g, 59.5%) as a colourless oil, which was used without further purification. Compound (3-2) was isolated as a single diastereoisomer. The stereocenter alpha to the amide is depicted as (S), on the expectation that hydrogenation occurred on the least hindered face of the alkene.

1H-NMR (400 MHz, DMSO, 23° C.) δ 1.16-1.32 (m, 4H), 1.34-1.47 (m, 4H), 1.51 (s, 2H), 1.71 (d, J=39.1 Hz, 2H), 2.08 (s, 1H), 2.94-3.03 (m, 2H), 3.37 (q, J=6.1 Hz, 5H), 3.68-4.1 (m, 9H). LCMS m/z ESI Expected 463.2, Observed 464.2 ([M+H]⁺).

DMAP (211 mg, 1.73 mmol) was added to (3-2) (400 mg, 0.86 mmol), (2R,3R,4R,5R,6R)-5-acetamido-2-(acetoxymethyl)-6-((6-aminohexyl)oxy)tetrahydro-2H-pyran-3,4-diyl diacetate (2-9) (1349 mg, 3.02 mmol), BOP (763 mg, 1.73 mmol) and DIEA (0.452 mL, 2.59 mmol) in DMF (10 mL). The resulting mixture was stirred at RT for 2 hours. The reaction was quenched with water (5 ml), the solvent was removed under reduced pressure. The crude product was purified by flash silica chromatography, elution gradient 7 to 10% MeOH in DCM. Pure fractions were evaporated to dryness to afford (2R,2′R,3R,3′R,4R,4′R,5R,5′R,6R,6′R)-((((2,2′-(((1R,2S,3R,5S)-3-(2-((6-(((2S,3S,4R,5R,6R)-3-acetamido-4,5-diacetoxy-6-(acetoxymethyl)tetrahydro-2H-pyran-2-yl)oxy)hexyl)amino)-2-oxoethoxy)-5-((6-hydroxyhexyl)(methyl)carbamoyl)cyclohexane-1,2-diyl)bis(oxy))bis(acetyl))bis(azanediyl))bis(hexane-6,1-diyl))bis(oxy))bis(5-acetamido-2-(acetoxymethyl)tetrahydro-2H-pyran-6,3,4-triyl) tetraacetate (3-3) (370 mg, 24.51%) as a yellow gum.

1H-NMR (400 MHz, DMSO, 25° C.) δ 1.22-1.26 (m, 14H), 1.45 (s, 14H), 1.71 (d, J=11.1 Hz, 2H), 1.77-1.78 (m, 11H), 1.89 (s, 9H), 1.99 (s, 9H), 2.10 (s, 9H), 2.78 (s, 1H), 2.93-3.03 (m, 7H), 3.42 (s, 10H), 3.66-3.69 (m, 4H), 3.89 (dd, J=11.7, 2.9 Hz, 3H), 3.92-3.99 (m, 6H), 4.02 (d, J=2.0 Hz, 8H), 4.97 (dd, J=11.2, 3.4 Hz, 3H), 5.03-5.12 (m, 3H), 5.21 (d, J=3.4 Hz, 3H), 6.95-7.01 (m, 1H), 7.70 (dd, J=9.3, 3.4 Hz, 6H), 7.75-7.87 (m, 5H), 8.19-8.24 (m, 1H). LCMS m/z Expected 1747.8, Observed 1749.6 ([M+H]⁺).

Chromosulfuric acid (0.172 mL, 0.34 mmol) was added to (3-3) (300 mg, 0.17 mmol) in acetone (10 mL) at 0° C. The resulting mixture was stirred at RT for 3 hours. The reaction was quenched with isopropyl alcohol (15 mL), the mixture was poured into ice water (20 mL). The solvent was removed under reduced pressure. The crude product was purified by flash C18-flash chromatography, elution gradient 0 to 60% MeCN in water (0.1% TFA). Pure fractions were evaporated to dryness to afford 6-((1S,3R,4S,5R)-3,4-bis(2-((6-(((2R,3R,4R,5R,6R)-3-acetamido-4,5-diacetoxy-6-(acetoxymethyl)tetrahydro-2H-pyran-2-yl)oxy)hexyl)amino)-2-oxoethoxy)-5-(2-((6-(((2S,3S,4R,5R,6R)-3-acetamido-4,5-diacetoxy-6-(acetoxymethyl)tetrahydro-2H-pyran-2-yl)oxy)hexyl)amino)-2-oxoethoxy)-N-methylcyclohexane-1-carboxamido)hexanoic acid (3-4) (110 mg, 36.4%) as a green solid.

1H-NMR (400 MHz, DMSO, 23° C.) δ 1.24 (s, 15H), 1.37-1.5 (m, 15H), 1.62-1.74 (m, 4H), 1.77 (s, 9H), 1.89 (s, 9H), 1.99 (s, 9H), 2.10 (s, 9H), 2.19 (t, J=6.1 Hz, 2H), 2.78 (s, 1H), 2.97 (s, 3H), 3.08 (s, 6H), 3.24 (s, 2H), 3.39 (dd, J=9.9, 6.6 Hz, 6H), 3.88 (ddd, J=19.4, 9.8, 6.8 Hz, 9H), 3.94-3.98 (m, 3H), 3.99-4.06 (m, 10H), 4.48 (d, J=8.5 Hz, 3H), 4.96 (dd, J=11.3, 3.4 Hz, 3H), 5.21 (d, J=3.4 Hz, 3H), 7.64-7.86 (m, 6H). LCMS m/z ESI⁺ Expected 1761.8, Observed 882.1 (z=2).

Perfluorophenyl 2,2,2-trifluoroacetate (0.054 mL, 0.31 mmol) was added to (3-4) (110 mg, 0.06 mmol) and DIEA (0.065 mL, 0.37 mmol) in DMF (8 mL) at RT. The resulting mixture was stirred at RT for 45 minutes. The reaction mixture was diluted with EtOAc (50 mL), and washed sequentially with 1M NaHSO4 (3×50 ml), saturated NaHCO₃ (2×50 ml), and saturated brine (2×50 ml). The organic layer was dried over Na2SO4, filtered and evaporated to dryness. The residue was dried by lyophilization to give (2R,2′R,3R,3′R,4R,4′R,5R,5′R,6R,6′R)-((((2,2′-(((1R,2S,3R,5S)-3-(2-((6-(((2S,3S,4R,5R,6R)-3-acetamido-4,5-diacetoxy-6-(acetoxymethyl)tetrahydro-2H-pyran-2-yl)oxy)hexyl)amino)-2-oxoethoxy)-5-(methyl(6-oxo-6-(perfluorophenoxy)hexyl)carbamoyl)cyclohexane-1,2-diyl)bis(oxy))bis(acetyl))bis(azanediyl))bis(hexane-6,1-diyl))bis(oxy))bis(5-acetamido-2-(acetoxymethyl)tetrahydro-2H-pyran-6,3,4-triyl) tetraacetate (3) (90 mg, 74.8%) as a white solid.

1H-NMR (400 MHz, DMSO, 23° C.) δ 1.14-1.31 (m, 22H), 1.34-1.55 (m, 16H), 1.68 (dt, J=14.0, 7.5 Hz, 6H), 1.77 (s, 8H), 1.89 (s, 7H), 1.99 (s, 8H), 2.10 (s, 8H), 2.78 (dt, J=7.4, 4.4 Hz, 3H), 2.98 (d, J=5.4 Hz, 2H), 3.02-3.17 (m, 5H), 3.24-3.31 (m, 2H), 3.41 (dd, J=10.1, 6.5 Hz, 3H), 3.53-3.78 (m, 5H), 3.79-3.98 (m, 8H), 3.99-4.27 (m, 8H), 4.3-4.61 (m, 3H), 4.79-5.08 (m, 3H), 5.21 (d, J=3.4 Hz, 3H), 7.59-8.01 (m, 5H), 11.45 (s, 1H). LCMS m/z ESI Expected 1927.8, Observed 1929.1 ([M+H]⁺).

### Synthesis of Precursor (4)

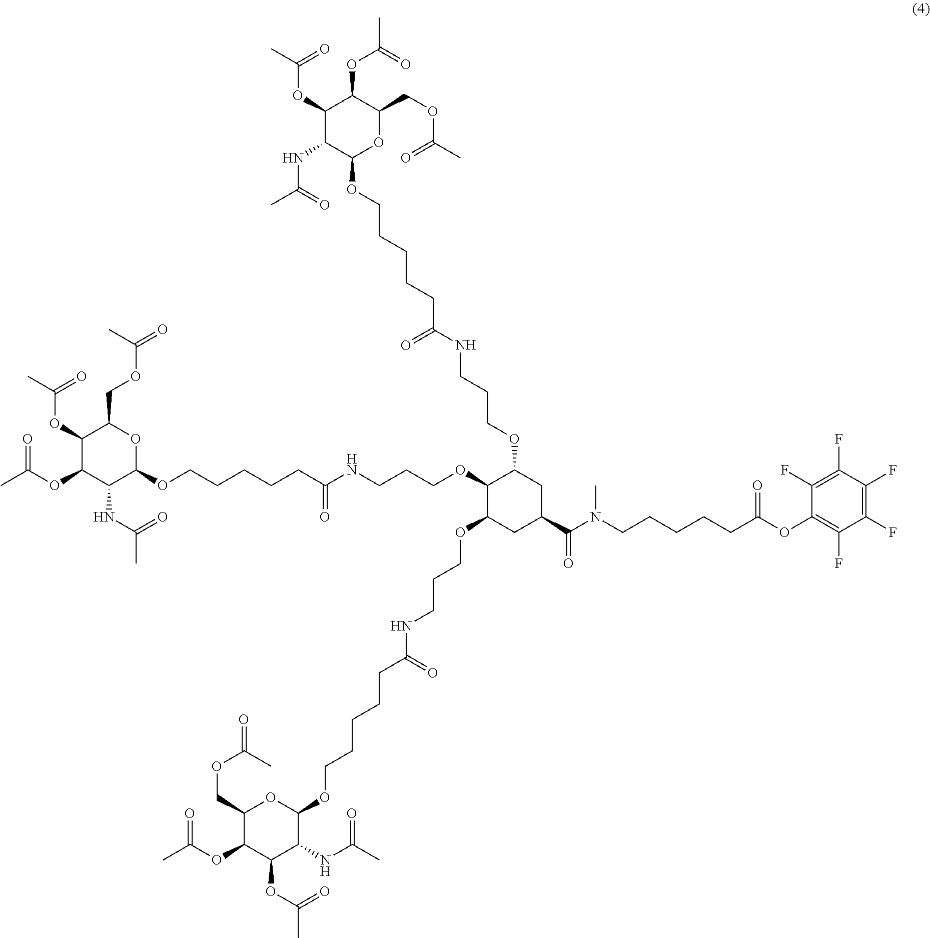 [SMILES: CC(=O)N[C@H]1[C@H](OCCCCCC(=O)NCCCO[C@@H]2C[C@H](C(=O)N(C)CCCCCC(=O)Oc3c(F)c(F)c(F)c(F)c3F)C[C@@H](OCCCNC(=O)CCCCCO[C@@H]3O[C@H](COC(C)=O)[C@H](OC(C)=O)[C@H](OC(C)=O)[C@H]3NC(C)=O)[C@@H]2OCCCNC(=O)CCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

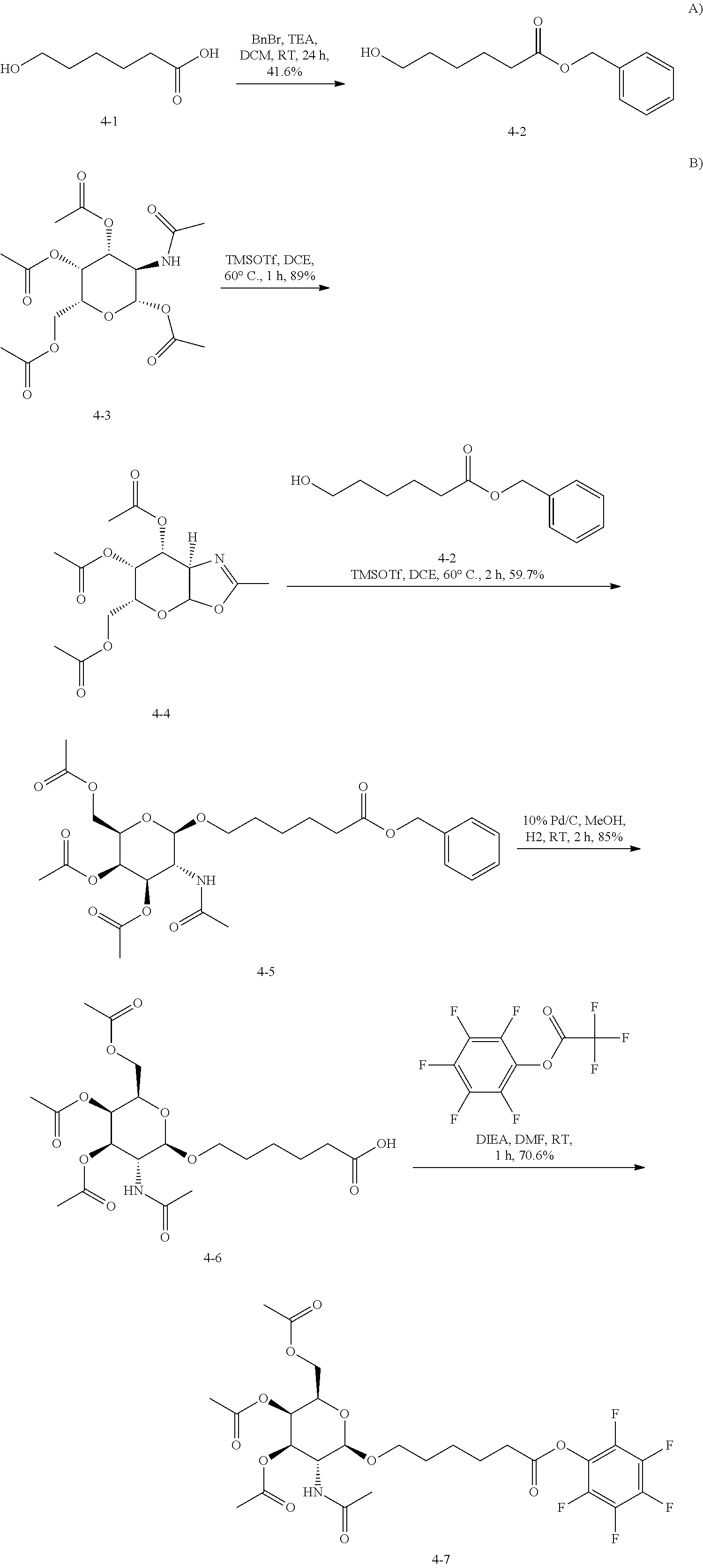

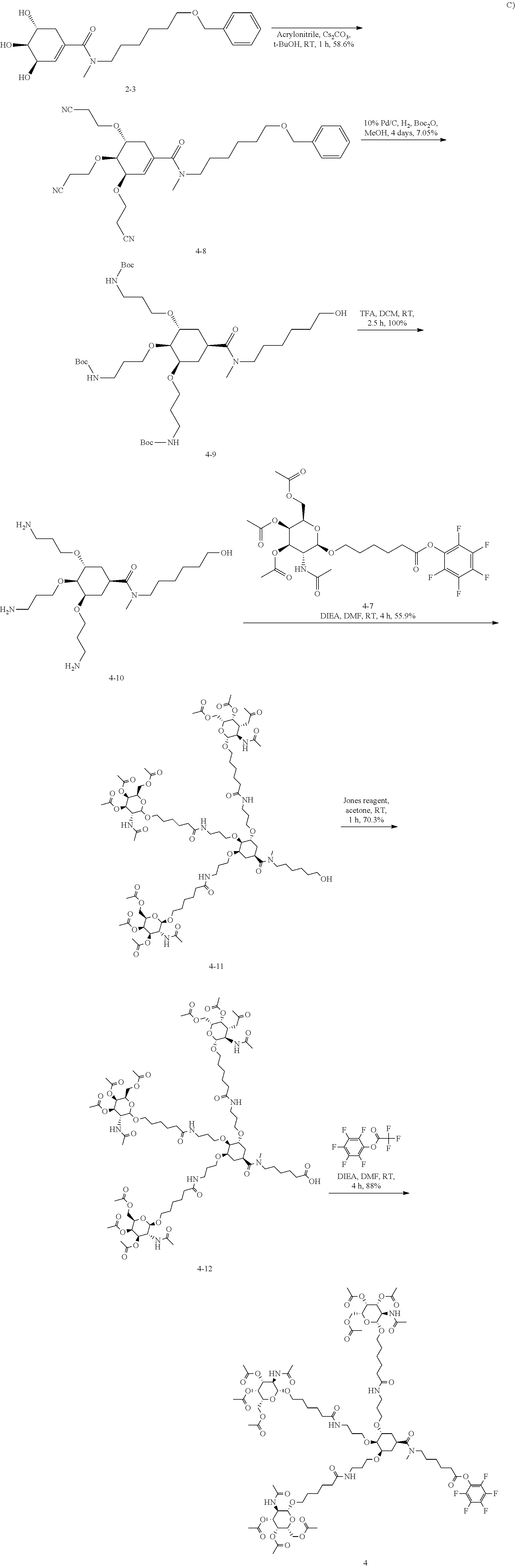

In part (A), (bromomethyl)benzene (10.78 mL, 90.80 mmol) was added to 6-hydroxyhexanoic acid (4-1) (10 g, 75.67 mmol) and TEA (31.6 mL, 227.00 mmol) in DCM (200 mL) at RT. The resulting mixture was stirred at RT for 24 hours. The reaction mixture was poured into water (250 ml), extracted with DCM (2×250 mL), the organic layer was dried over Na2SO4, filtered and evaporated to dryness. The crude product was purified by flash silica chromatography, elution gradient 10 to 70% EtOAc in petroleum ether. Pure fractions were evaporated to dryness to afford benzyl 6-hydroxyhexanoate (4-2) (7.00 g, 41.6%) as a colourless oil.

1H-NMR (300 MHz, DMSO, 24° C.) δ 1.22-1.34 (m, 2H), 1.34-1.47 (m, 2H), 1.55 (p, J=7.4 Hz, 2H), 2.35 (t, J=7.4 Hz, 2H), 3.37 (t, J=6.4 Hz, 2H), 4.35 (brs, 1H), 5.09 (s, 2H), 7.23-7.52 (m, 5H). LCMS m/z ESI Expected 222.1, Observed 223.2 ([M+H]⁺).

In part (B), TMs-OTf (5.10 mL, 28.25 mmol) was added to (2S,3R,4R,5R,6R)-3-acetamido-6-(acetoxymethyl)tetrahydro-2H-pyran-2,4,5-triyl triacetate (4-3) (10 g, 25.68 mmol) in DCE (100 mL) at RT. The resulting mixture was stirred at 60° C. for 1 hours. After cooled to RT, TEA (5.37 mL, 38.53 mmol) was added dropwise to the mixture and stirred for 10 minutes. The reaction mixture was diluted with DCM (50 mL), and washed sequentially with saturated NaHCO₃ (100 mL), saturated brine (100 mL). The organic layer was dried over Na2SO4, filtered and evaporated to dryness to afford (5R,6R,7R,7aR)-5-(acetoxymethyl)-2-methyl-3a,6,7,7a-tetrahydro-5H-pyrano[3,2-d]oxazole-6,7-diyl diacetate (4-4) (7.50 g, 89%) as a pale yellow oil. The product was used in the next step directly without further purification.

1H-NMR (300 MHz, DMSO, 25° C.) δ 1.96 (d, J=1.5 Hz, 3H), 2.02 (d, J=2.2 Hz, 6H), 2.07 (s, 3H), 3.92-4 (m, 1H), 4-4.17 (m, 2H), 4.27 (ddd, J=7.5, 5.1, 2.8 Hz, 1H), 4.89 (dd, J=6.8, 3.9 Hz, 1H), 5.25 (dd, J=3.9, 2.9 Hz, 1H), 6.06 (d, J=7.0 Hz, 1H). LCMS m/z ESI Expected 329.1, Observed 330.2 ([M+H]⁺).

(4-2) (5.57 g, 25.05 mmol) was added to (4-4) (7.5 g, 22.78 mmol) and 4 A molecular sieves (3.6 g, 0.00 mmol) in DCE (75 mL) at RT. TMs-OTf (4.12 mL, 22.78 mmol) was added after the reaction had been stirred for 30 minutes at 60° C. The resulting mixture was stirred at 60° C. for further 2 hours. The reaction mixture was poured into saturated NaHCO₃ (200 mL), extracted with DCM (2×200 mL), the organic layer was dried over Na2SO4, filtered and evaporated to dryness to afford (2R,3R,4R,5R)-5-acetamido-2-(acetoxymethyl)-6-((6-(benzyloxy)-6-oxohexyl)oxy)tetrahydro-2H-pyran-3,4-diyl diacetate (4-5) (7.50 g, 59.7%) as a yellow oil. The product was used in the next step directly without further purification.

1H-NMR (300 MHz, DMSO, 24° C.) δ 1.29 (q, J=8.6 Hz, 2H), 1.49 (dq, J=20.3, 7.0, 6.4 Hz, 4H), 1.90 (s, 3H), 2.00 (d, J=1.1 Hz, 6H), 2.10 (s, 3H), 2.34 (t, J=7.3 Hz, 2H), 3.41 (dt, J=9.9, 6.5 Hz, 1H), 3.69 (dt, J=11.6, 6.1 Hz, 1H), 3.8-3.9 (m, 1H), 4.03 (d, J=7.1 Hz, 4H), 4.19-4.31 (m, 1H), 4.49 (d, J=8.5 Hz, 1H), 4.97 (dd, J=11.3, 3.4 Hz, 1H), 5.23 (dd, J=10.7, 3.4 Hz, 1H), 7.36 (d, J=3.8 Hz, 5H), 7.81 (d, J=9.2 Hz, 1H). LCMS m/z ESI Expected 551.2, Observed 552.4 ([M+H]⁺).

(4-5) (7.0 g, 12.69 mmol) and Pd/C (10%, 50 w/w % water) (1.351 g, 1.27 mmol) in MeOH (80 mL) was stirred under an atmosphere of hydrogen at 1 atm and RT for 2 hours. The reaction mixture was filtered through celite. The solvent was removed under reduced pressure to give 6-(((2R,3R,4R,5R,6R)-3-acetamido-4,5-diacetoxy-6-(acetoxymethyl)tetrahydro-2H-pyran-2-yl)oxy)hexanoic acid (4-6) (5.00 g, 85%) as a yellow oil. The product was used in the next step directly without further purification.

1H-NMR (300 MHz, DMSO, 22° C.) δ 1.23-1.32 (m, 2H), 1.48 (t, J=7.7 Hz, 4H), 1.89 (s, 3H), 2.00 (s, 3H), 2.07 (s, 3H), 2.10 (s, 3H), 2.18 (t, J=7.4 Hz, 2H), 3.3-3.47 (m, 2H), 3.69 (dt, J=9.8, 6.1 Hz, 1H), 3.87 (dt, J=11.3, 8.8 Hz, 1H), 4.14-4.29 (m, 1H), 4.49 (d, J=8.5 Hz, 1H), 4.88-5.12 (m, 2H), 5.21 (d, J=3.4 Hz, 1H), 7.81 (d, J=9.2 Hz, 1H), 11.96 (brs, 1H). LCMS m/z ESI Expected 461.1, Observed 484.2 ([M+Na]f).

Perfluorophenyl 2,2,2-trifluoroacetate (1.821 g, 6.50 mmol) was added to (4-6) (2.5 g, 5.42 mmol) and DIEA (1.183 mL, 6.77 mmol) in DMF (50 mL) at RT. The resulting mixture was stirred at RT for 1 hour. The reaction mixture was diluted with DCM (200 mL), and washed sequentially with 1M NaHSO4 (3×100 mL), saturated NaHCO₃ (3×100 mL), and saturated brine (2×100 mL). The organic layer was dried over Na2SO4, filtered and evaporated to dryness to afford (2R,3R,4R,5R,6R)-5-acetamido-2-(acetoxymethyl)-6-((6-oxo-6-(perfluorophenoxy)hexyl)oxy)tetrahydro-2H-pyran-3,4-diyl diacetate (4-7) (2.400 g, 70.6%) as a pale yellow oil which solidified on standing. The product was used in the next step directly without further purification.

1H-NMR (500 MHz, DMSO, 25° C.) δ 1.32-1.45 (m, 2H), 1.53 (p, J=6.7 Hz, 2H), 1.6-1.71 (m, 2H), 1.77 (d, J=3.4 Hz, 3H), 1.90 (s, 3H), 2.00 (s, 3H), 2.11 (s, 3H), 2.72-2.85 (m, 2H), 3.41-3.77 (m, 2H), 3.88 (dq, J=10.8, 8.4 Hz, 1H), 4.04 (s, 2H), 4.09-4.28 (m, 1H), 4.50 (t, J=7.2 Hz, 1H), 4.87-5.01 (m, 1H), 5.22 (t, J=2.9 Hz, 1H), 7.82 (dd, J=9.2, 4.8 Hz, 1H). LCMS m/z ESI Expected 627.1, Observed 650.2 ([M+Na]⁺).

In part (C), acrylonitrile (126 mL, 1907.38 mmol) was added to (3R,4S,5R)—N-(6-(benzyloxy)hexyl)-3,4,5-trihydroxy-N-methylcyclohex-1-ene-1-carboxamide (2-3) (12 g, 31.79 mmol) and Cs₂CO₃ (31.1 g, 95.37 mmol) in tert-butanol (200 mL) under argon. The resulting mixture was stirred at RT for 1 hour. The reaction mixture was diluted with EtOAc, the solid was filtered out and the filtration was evaporated to dryness. The crude product was purified by flash silica chromatography, elution gradient 0 to 100% EtOAc in petroleum ether then 0 to 20% MeOH in EtOAc. Pure fractions were evaporated to dryness to afford (3R,4S,5R)—N-(6-(benzyloxy)hexyl)-3,4,5-tris(2-cyanoethoxy)-N-methylcyclohex-1-ene-1-carboxamide (4-8) (10.00 g, 58.6%) as a yellow oil.

1H-NMR (400 MHz, DMSO, 17° C.) δ 1.22 (h, J=6.1 Hz, 2H), 1.33 (t, J=7.6 Hz, 2H), 1.53 (td, J=13.7, 6.8 Hz, 4H), 2.08 (s, 3H), 2.52-2.61 (m, 1H), 2.76 (q, J=9.6, 6.0 Hz, 7H), 2.93 (s, 2H), 3.2-3.36 (m, 2H), 3.41 (t, J=6.5 Hz, 2H), 3.67-3.89 (m, 6H), 4.18 (t, J=3.7 Hz, 1H), 4.45 (s, 2H), 5.62-5.67 (m, 1H), 7.25-7.4 (m, 5H). LCMS m/z ESI Expected 536.3, Observed 537.4 ([M+H]⁺).

(4-8) (2 g, 3.73 mmol), 10% Pd/C (7.93 g, 7.45 mmol) and Boc-anhydride (4.33 mL, 18.63 mmol) in MeOH (130 mL) were stirred under an atmosphere of hydrogen at 20 atm at 50° C. for 2 days. The reaction mixture was filtered and the filtrate was concentrated to dryness, the residue was dissolved in MeOH (200 mL), the same amount of Pd/C (10%) and Boc-anhydride were added, stirred under an atmosphere of hydrogen at 20 atm at 50° C. for further 2 days. The reaction mixture was filtered through celite. The filtration was concentrated to dryness. The crude product was purified by flash C18-flash chromatography, elution gradient 0 to 60% MeCN in water (0.1% NH4HCO3). Pure fractions were evaporated to dryness to afford di-tert-butyl ((((1R,3R)-2-(3-((tert-butoxycarbonyl)amino)propoxy)-5-((6-hydroxyhexyl)(methyl)carbamoyl)cyclohexane-1,3-diyl)bis(oxy))bis(propane-3,1-diyl))dicarbamate (4-9) (0.200 g, 7.05%) as a colorless liquid.

1H-NMR (300 MHz, DMSO, 24° C.) δ 1.20 (d, J=24.3 Hz, 6H), 1.37 (s, 29H), 1.58 (s, 10H), 2.52 (s, 1H), 2.77 (s, 3H), 2.98 (d, J=7.8 Hz, 6H), 3.23 (t, J=7.3 Hz, 2H), 3.35-3.54 (m, 8H), 3.54-3.68 (m, 3H), 4.33 (t, J=5.2 Hz, 1H), 6.61-6.82 (m, 2H). One proton has been exchanged. LCMS m/z ESI Expected 760.5, Observed 761.3 ([M+H]⁺).

The solution of the (4-9) (181 mg, 0.24 mmol) in DCM/TFA=1:1 (12 mL) was stirred 2.5 hours at RT. The solvent was removed under reduced pressure to afford (1S,3R,4S,5R)-3,4,5-tris(3-aminopropoxy)-N-(6-hydroxyhexyl)-N-methylcyclohexane-1-carboxamide (4-10) (110 mg, 100%) as a yellow oil. The product was used in the next step directly without further purification.

1H-NMR (300 MHz, DMSO, 24° C.) δ 1.24 (d, J=2.7 Hz, 3H), 1.31-1.44 (m, 3H), 1.57 (q, J=11.1, 9.8 Hz, 4H), 1.75 (dq, J=17.2, 7.8, 7.1 Hz, 9H), 2.53-2.63 (m, 1H), 2.78 (s, 2H), 2.87 (p, J=7.6 Hz, 5H), 2.96 (s, 2H), 3.24 (t, J=7.2 Hz, 2H), 3.50 (q, J=6.1 Hz, 5H), 3.58-3.77 (m, 4H), 4.38 (td, J=6.6, 2.9 Hz, 2H), 7.80 (brs, 6H). LCMS m/z ESI Expected 460.3, Observed 461.3 ([M+H]⁺).

DIEA (0.279 mL, 1.60 mmol) was added to (4-10) and (4-7) (627 mg, 0.80 mmol) in DMF (25 mL) at RT. The resulting mixture was stirred at RT for 4 hours. The solvent was removed under reduced pressure. The crude product was purified by flash C18-flash chromatography, elution gradient 0 to 50% MeCN in water (0.1% FA). Pure fractions were evaporated to dryness to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexanoylamino]propoxy]-5-[6-hydroxyhexyl(methyl)carbamoyl]cyclohexoxy]propylamino]-6-oxo-hexoxy]tetrahydropyran-2-yl]methyl acetate (4-11) (200 mg, 55.9%) as a yellow solid.

1H-NMR (300 MHz, DMSO, 24° C.) δ 1.17-1.32 (m, 14H), 1.32-1.61 (m, 1), 1.77 (s, 9H), 1.90 (s, 9H), 2.00 (s, 9H), 2.11 (s, 8H), 2.16-2.33 (m, 8H), 2.71 (s, 1H), 2.86 (d, J=94.2 Hz, 3H), 3.03 (s, 6H), 3.24 (s, 3H), 3.36-3.46 (m, 4H), 3.55-3.76 (m, 14H), 3.87 (q, J=9.4 Hz, 3H), 4.03 (q, J=4.0 Hz, 8H), 4.49 (d, J=8.5 Hz, 3H), 4.97 (dd, J=11.2, 3.4 Hz, 3H), 5.22 (d, J=3.5 Hz, 3H), 7.79 (dd, J=30.8, 7.3 Hz, 6H), 11.99 (brs, 1H). LCMS m/z ESI Expected 1789.9, Observed 1790.8 ([M+H]⁺).

Chromosulfuric acid in H₂SO₄ (0.098 mL, 0.20 mmol) was added to (4-11) (175 mg, 0.10 mmol) in acetone (20 mL) at 0° C. The resulting mixture was stirred at RT for 1 hour. The reaction mixture was quenched with i-PrOH (10 ml), the solvent was removed under reduced pressure. The crude product was diluted with DCM (50 ml), washed sequentially with water (50 mL), saturated NaHCO₃ (2×50 mL), and saturated brine (50 mL). The organic layer was dried over Na2SO4, filtered and evaporated to dryness. The crude product was purified by flash C18-flash chromatography, elution gradient 0 to 40% MeCN in water (0.1% FA). Pure fractions were evaporated to dryness to afford 6-((3R,5R)-3,4,5-tris(3-(6-(((2R,3R,4R,5R,6R)-3-acetamido-4,5-diacetoxy-6-(acetoxymethyl)tetrahydro-2H-pyran-2-yl)oxy)hexanamido)propoxy)-N-methylcyclohexane-1-carboxamido)hexanoic acid (4-12) (124 mg, 70.3%) as a yellow solid.

1H-NMR (500 MHz, DMSO, 25° C.) δ 1.17-1.32 (m, 14H), 1.32-1.61 (m, 1), 1.77 (s, 9H), 1.90 (s, 9H), 2.00 (s, 9H), 2.11 (s, 8H), 2.16-2.33 (m, 8H), 2.71 (s, 1H), 2.86 (d, J=94.2 Hz, 3H), 3.03 (s, 6H), 3.24 (s, 3H), 3.36-3.46 (m, 4H), 3.55-3.76 (m, 11H), 3.87 (q, J=9.4 Hz, 3H), 4.03 (q, J=4.0 Hz, 8H), 4.49 (d, J=8.5 Hz, 3H), 4.97 (dd, J=11.2, 3.4 Hz, 3H), 5.22 (d, J=3.5 Hz, 3H), 7.79 (dd, J=30.8, 7.3 Hz, 6H), 11.99 (brs, 1H). LCMS m/z ESI Expected 1803.8, Observed 1804.6 ([M+H]⁺).

Perfluorophenyl 2,2,2-trifluoroacetate (0.023 mL, 0.13 mmol) was added to (4-12) (80 mg, 0.04 mmol) and DIEA (0.023 mL, 0.13 mmol) in DMF (50 mL) at RT. The resulting mixture was stirred at RT for 20 minutes. The reaction mixture was diluted with EtOAc (50 mL), washed sequentially with 1M NaHSO4 (4×50 mL), saturated NaHCO₃ (2×50 mL), and saturated brine (50 mL). The organic layer was dried over Na2SO4, filtered and evaporated to dryness. The residue was dried by lyophilization to give (2,3,4,5,6-pentafluorophenyl) 6-[methyl-[(3R,5R)-3,4,5-tris[3-[6-[(2R,3R,4R,5R,6R)-3-acetamido-4,5-diacetoxy-6-(acetoxymethyl)tetrahydropyran-2-yl]oxyhexanoylamino]propoxy]cyclohexanecarbonyl]amino]hexanoate (4) (67.0 mg, 77%) as a gray solid.

1H-NMR (400 MHz, DMSO, 21C) 1.19-1.31 (m, 14H), 1.39-1.5 (m, 13H), 1.52-1.67 (m, 13H), 1.77 (s, 8H), 1.89 (s, 8H), 1.97-2.06 (m, 13H), 2.10 (s, 8H), 2.69-2.85 (m, 5H), 2.88-3 (m, 7H), 3.03-3.16 (m, 6H), 3.53-3.76 (m, 10H), 3.79-3.94 (m, 3H), 3.96-4.07 (m, 9H), 4.48 (d, J=8.5 Hz, 3H), 4.96 (dd, J=11.2, 3.4 Hz, 3H), 5.21 (d, J=3.4 Hz, 3H), 7.69-7.87 (m, 5H). One proton has been exchanged. LCMS m/z ESI⁺ Expected 1969.8, Observed 986.2 (z=2).

Alternatively, perfluorophenyl 2,2,2-trifluoroacetate (0.029 mL, 0.18 mmol) was added to (4-12) (100 mg, 0.06 mmol) and DIEA (0.048 mL, 0.28 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (4) (108.0 mg, 88%) as beige solid. The product was used in the next reaction without any further purification. LCMS m/z Expected 1969.863, Observed 986.8 [M+H]+(z=2).

### Synthesis of Precursor (5)

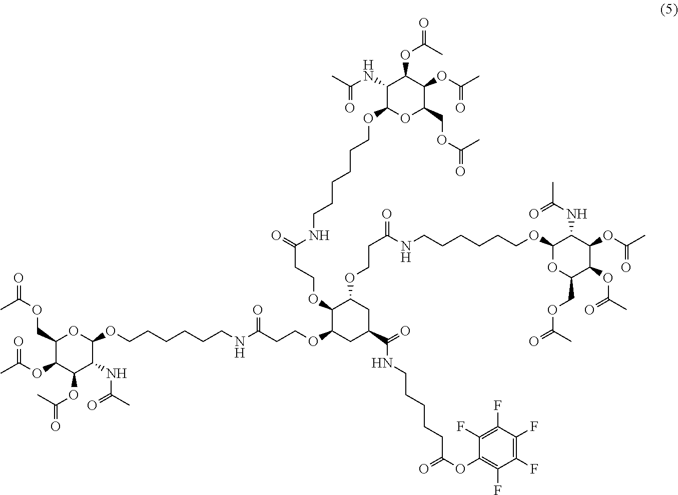 [SMILES: CC(=O)N[C@H]1[C@H](OCCCCCCNC(=O)CCO[C@@H]2C[C@H](C(=O)NCCCCCC(=O)Oc3c(F)c(F)c(F)c(F)c3F)C[C@@H](OCCC(=O)NCCCCCCO[C@@H]3O[C@H](COC(C)=O)[C@H](OC(C)=O)[C@H](OC(C)=O)[C@H]3NC(C)=O)[C@@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

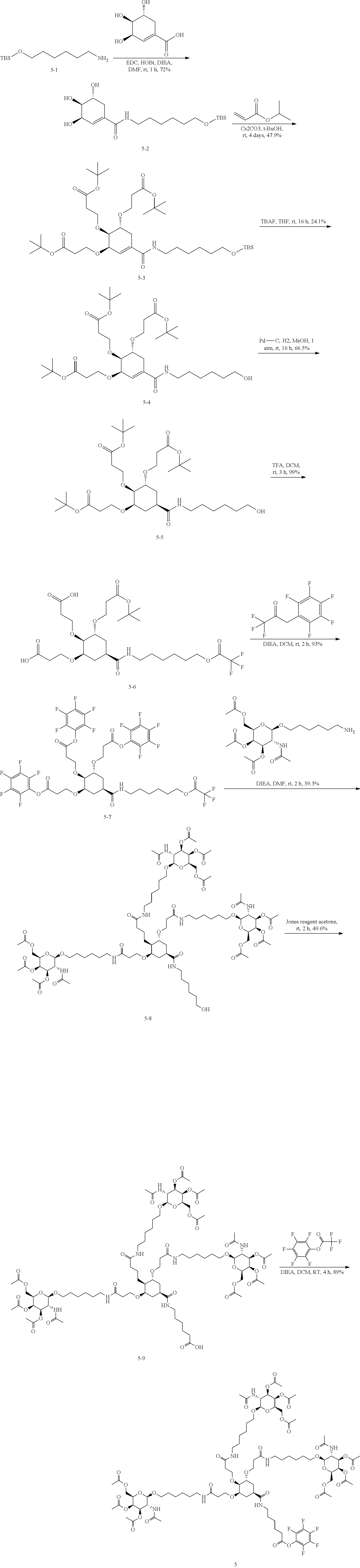

6-((tert-butyldimethylsilyl)oxy)hexan-1-amine (5 g, 21.60 mmol) was added to (3R,4S,5R)-3,4,5-trihydroxycyclohex-1-ene-1-carboxylic acid (4.14 g, 23.76 mmol), EDC (8.70 g, 45.37 mmol), HOBt (6.62 g, 43.20 mmol) and DIEA (11.32 mL, 64.81 mmol) in DMF (50 mL). The resulting mixture was stirred at rt for 1 hour. The solvent was removed under reduced pressure to afford the crude product. The crude product was purified by flash silica chromatography, elution gradient 0 to 5% MeOH in DCM. Pure fractions were evaporated to dryness to afford (3R,4S,5R)—N-(6-((tert-butyldimethylsilyl)oxy)hexyl)-3,4,5-trihydroxycyclohex-1-ene-1-carboxamide (5-2) (4.40 g, 72%) as a yellow liquid.

1H-NMR (400 MHz, DMSO, 24° C.) δ 0.10 (d, J=1.7 Hz, 6H), 0.85 (d, J=6.3 Hz, 9H), 1.24 (s, 2H), 1.33-1.49 (m, 6H), 1.94-2.02 (m, 2H), 3.07-3.09 (m, 2H), 3.56 (m, 4H), 4.03 (q, J=7.1 Hz, 1H), 4.17 (d, J=3.8 Hz, 1H), 6.27 (dd, J=3.4, 1.7 Hz, 1H), 7.23-7.32 (m, 2H), 7.80 (q, J=1.8, 1.4 Hz, 1H). LCMS m/z Expected 387.2, Observed 388.4.

Cs₂CO₃ (24.13 g, 74.07 mmol) was added to tert-Butyl acrylate (197 mL, 1346.78 mmol) and (3R,4S,5R)—N-(6-((tert-butyldimethylsilyl)oxy)hexyl)-3,4,5-trihydroxycyclohex-1-ene-1-carboxamide (5-2) (8.7 g, 22.45 mmol) in t-BuOH (800 mL). The resulting mixture was stirred at RT for 3 days. The reaction mixture was diluted with EtOAc (1 L), the mixture was filtered through a Celite pad. The solvent was removed under reduced pressure to afford the crude product. The crude product was purified by flash silica chromatography, elution gradient 0 to 50% EtOAc in petroleum ether. Pure fractions were evaporated to dryness to afford tri-tert-butyl 3,3′,3″-(((1R,2S,3R)-5-((6-((tert-butyldimethylsilyl)oxy)hexyl)carbamoyl)cyclohex-4-ene-1,2,3-triyl)tris(oxy))tripropionate (5-3) (8.30 g, 47.9%) as a yellow oil. The crude product was used in the next step directly without further purification.

1H-NMR (400 MHz, DMSO, 24° C.) δ 0.86 (s, 9H), 1.12 (s, 2H), 1.40 (dd, J=4.3, 1.3 Hz, 31H), 1.47-1.58 (m, 2H), 2.36-2.43 (m, 8H), 3.06 (d, J=6.6 Hz, 2H), 3.49 (t, J=6.2 Hz, 1H), 3.65-3.77 (m, 9H), 3.92 (dt, J=21.1, 4.6 Hz, 1H), 6.26-6.31 (m, 1H), 7.87 (tdd, J=8.2, 5.6, 2.8 Hz, 1H). Three protons were exchanged. LCMS m/z Expected 771.5, Observed 772.4.

TBAF (1M in THF) (9.45 mL, 9.45 mmol) was added to tri-tert-butyl 3,3′,3″-(((1R,2S,3R)-5-((6-((tert-butyldimethylsilyl)oxy)hexyl)carbamoyl)cyclohex-4-ene-1,2,3-triyl)tris(oxy))tripropionate (5-3) (7.3 g, 9.45 mmol) in THE (80 mL). The resulting mixture was stirred at RT for 16 hours. The solvent was removed under reduced pressure to afford the crude product. The crude product was purified by flash silica chromatography, elution gradient 0 to 80% EtOAc in petroleum ether. Pure fractions were evaporated to dryness to afford tri-tert-butyl 3,3′,3″-(((1R,2S,3R)-5-((6-hydroxyhexyl)carbamoyl)cyclohex-4-ene-1,2,3-triyl)tris(oxy))tripropionate (5-4) (1.500 g, 24.12%) as a colorless oil.

1H-NMR (400 MHz, DMSO, 23° C.) δ 1.18-1.31 (m, 4H), 1.37-1.45 (m, 29H), 2.16 (dd, J=18.2, 3.6 Hz, 1H), 2.32-2.47 (m, 7H), 3.06 (q, J=6.6 Hz, 2H), 3.17 (d, J=5.2 Hz, 3H), 3.37 (m, 2H), 3.73 (m, 6H), 4.10 (q, J=5.2 Hz, 1H), 4.33 (t, J=5.1 Hz, 1H), 6.29 (d, J=2.5 Hz, 1H), 7.87 (q, J=5.8, 4.5 Hz, 1H). One proton was exchanged. LCMS m/z Expected 657.4, Observed 658.5.

Tri-tert-butyl 3,3′,3″-(((1R,2S,3R)-5-((6-hydroxyhexyl)carbamoyl)cyclohex-4-ene-1,2,3-triyl)tris(oxy))tripropionate (5-4) (1.5 g, 2.28 mmol) and Pd—C(10%) (0.485 g, 0.46 mmol) in MeOH (30 mL) was stirred under an atmosphere of hydrogen at 1 atm and rt for 16 hours.

The reaction mixture was filtered through celite. The solvent was removed under reduced pressure to afford crude product. The crude product was purified by flash silica chromatography, elution gradient 10 to 70% EtOAc in petroleum ether. Pure fractions were evaporated to dryness to afford tri-tert-butyl 3,3′,3″-(((1R,2S,3R,5S)-5-((6-hydroxyhexyl)carbamoyl)cyclohexane-1,2,3-triyl)tris(oxy))tripropionate (5-5) (1.000 g, 66.5%) as a colorless oil.

1H-NMR (400 MHz, DMSO, 21° C.) δ 1.05-1.3 (m, 5H), 1.35-1.45 (m, 30H), 1.47-1.67 (m, 4H), 2.18-2.47 (m, 7H), 2.99 (q, J=6.5 Hz, 2H), 3.35-3.45 (m, 3H), 3.47-3.72 (m, 7H), 3.78 (m, 1H), 4.33 (t, J=5.2 Hz, 1H), 7.69 (q, J=6.5, 5.6 Hz, 1H). LCMS m/z Expected 659.4, Observed 660.2.

TFA (3.15 mL, 40.92 mmol) was added to tri-tert-butyl 3,3′,3″-(((1R,2S,3R,5S)-5-((6-hydroxyhexyl)carbamoyl)cyclohexane-1,2,3-triyl)tris(oxy))tripropionate (5-5) (0.9 g, 1.36 mmol) in DCM (10 mL) at rt. The resulting mixture was stirred at rt for 3 hours. The solvent was removed under reduced pressure to afford 3,3′,3″-(((1R,2S,3R,5S)-5-((6-(2,2,2-trifluoroacetoxy)hexyl)carbamoyl)cyclohexane-1,2,3-triyl)tris(oxy))tripropionic acid (5-6) (0.790 g, 99%) as a yellow oil. The product was used in the next step directly without further purification.

1H-NMR (300 MHz, DMSO, 23° C.) δ 1.26-1.4 (m, 6H), 1.46-1.63 (m, 4H), 1.64-1.72 (m, 2H), 2.29 (p, J=6.1 Hz, 1H), 2.35-2.49 (m, 7H), 2.99 (q, J=6.4 Hz, 2H), 3.41 (d, J=8.3 Hz, 1H), 3.59-3.67 (m, 5H), 3.72-3.83 (m, 2H), 4.37 (t, J=6.6 Hz, 2H), 7.70 (t, J=5.5 Hz, 1H), 12.61 (s, 3H). LCMS m/z Expected 587.2, Observed 588.2.

Perfluorophenyl 2,2,2-trifluoroacetate (0.921 mL, 5.36 mmol) was added to 3,3′,3″-(((1R,2S,3R,5S)-5-((6-(2,2,2-trifluoroacetoxy)hexyl)carbamoyl)cyclohexane-1,2,3-triyl)tris(oxy))tripropionic acid (5-6) (0.7 g, 1.19 mmol) and DIEA (1.353 mL, 7.74 mmol) in DCM (20 mL) at rt. The resulting mixture was stirred at rt for 2 hours. The reaction mixture was diluted with DCM (20 ml) and washed sequentially with 1 M NaHSO4 (2×25 ml), saturated NaHCO₃ (2×15 ml), and saturated brine (30 ml). The organic layer was dried over Na2SO4, filtered and evaporated to afford tris(perfluorophenyl) 3,3′,3″-(((1R,2S,3R,5S)-5-((6-(2,2,2-trifluoroacetoxy)hexyl)carbamoyl)cyclohexane-1,2,3-triyl)tris(oxy))tripropionate (5-7) (1.200 g, 93%) as a yellow oil. The product was used in the next step directly without further purification.

1H-NMR (300 MHz, DMSO, 23° C.) δ 1.27-1.43 (m, 6H), 1.53-1.72 (m, 6H), 2.32 (d, J=10.1 Hz, 1H), 2.87-3.11 (m, 11H), 3.74-3.92 (m, 5H), 3.98 (m, 1H), 4.36 (t, J=6.6 Hz, 2H), 7.71 (t, J=5.6 Hz, 1H). LCMS m/z Expected 1085.1, Observed 1086.0.

N-ethyl-N-isopropylpropan-2-amine (1.126 mL, 6.45 mmol) was added to tris(perfluorophenyl) 3,3′,3″-(((1R,2S,3R,5S)-5-((6-(2,2,2-trifluoroacetoxy)hexyl)carbamoyl)cyclohexane-1,2,3-triyl)tris(oxy))tripropionate (5-7) (700 mg, 0.64 mmol) and (2R,3R,4R,5R)-5-acetamido-2-(acetoxymethyl)-6-((6-aminohexyl)oxy)tetrahydro-2H-pyran-3,4-diyl diacetate (1152 mg, 2.58 mmol) in DMF (10 mL). The resulting mixture was stirred at rt for 2 hours. The reaction mixture was diluted with water (15 ml). The solvent was removed under reduced pressure to afford the crude product. The crude product was purified by flash C18-flash chromatography, elution gradient 0 to 50% MeCN in water (0.1% FA). Pure fractions were evaporated to dryness to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(6-hydroxyhexylcarbamoyl)cyclohexoxy]propanoylamino]hexoxy]tetrahydropyran-2-yl]methyl acetate (5-8) (450 mg, 39.3%) as a yellow solid.

1H NMR (300 MHz, DMSO, 23° C.) δ 1.25 (s, 16H), 1.47 (t, J=26.6 Hz, 17H), 1.78 (s, 9H), 1.90 (s, 9H), 2.00 (s, 10H), 2.10 (d, J=8.4 Hz, 12H), 2.29 (d, J=7.7 Hz, 5H), 3.02 (d, J=6.4 Hz, 5H), 3.17 (q, J=6.5 Hz, 3H), 3.41 (d, J=9.6 Hz, 10H), 3.53-3.79 (m, 9H), 3.87 (q, J=9.3 Hz, 3H), 4.03 (s, 9H), 4.49 (d, J=8.4 Hz, 3H), 4.97 (dd, J=11.2, 3.4 Hz, 3H), 5.22 (d, J=3.4 Hz, 3H), 7.48-8.16 (m, 6H), 9.41 (s, 1H). LCMS m/z Expected 1775.8, Observed 1776.8 ([M+H]⁺).

Chromosulfuric acid (0.225 mL, 0.45 mmol) was added to [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(6-hydroxyhexylcarbamoyl)cyclohexoxy]propanoylamino]hexoxy]tetrahydropyran-2-yl]methyl acetate (5-8) (400 mg, 0.23 mmol) in acetone (10 mL) at 0° C. The resulting mixture was stirred at rt for 2 hours. The reaction mixture was quenched with i-PrOH (20 ml), the mixture was diluted with DCM (100 ml) and washed sequentially with saturated NaHCO₃ (2×120 ml) and saturated brine (2×120 ml). The organic layer was dried over Na2SO4, filtered, and evaporated to afford crude product. The crude product was purified by flash C18-flash chromatography, elution gradient 0 to 40% MeCN in water (0.1% FA). Pure fractions were evaporated to dryness to afford 6-((3R,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohexane-1-carboxamido)hexanoic acid (5-9) (200 mg, 49.6%) as a yellow solid.

1H NMR (500 MHz, DMSO, 23° C.) δ 1.25 (d, J=5.6 Hz, 15H), 1.37 (q, J=7.0 Hz, 8H), 1.46 (p, J=6.9, 6.5 Hz, 8H), 1.53 (d, J=24.7 Hz, 4H), 1.78 (s, 9H), 1.90 (s, 9H), 2.00 (s, 9H), 2.11 (s, 10H), 2.27 (ddt, J=19.8, 14.7, 6.9 Hz, 7H), 3.01 (dp, J=18.6, 6.4, 5.9 Hz, 8H), 3.41 (dd, J=10.1, 6.3 Hz, 4H), 3.51-3.75 (m, 11H), 3.87 (q, J=9.5 Hz, 3H), 4.02 (q, J=3.9 Hz, 9H), 4.50 (dd, J=8.5, 3.4 Hz, 3H), 4.98 (dt, J=11.4, 2.9 Hz, 3H), 5.22 (d, J=3.4 Hz, 3H), 7.6-8.04 (m, 7H). LCMS m/z Expected 1789.8, Observed 1791.8 ([M+H]⁺).

Perfluorophenyl 2,2,2-trifluoroacetate (0.032 mL, 0.18 mmol) was added to (5-9) (110 mg, 0.06 mmol) and DIEA (0.054 mL, 0.31 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (5) (107.0 mg, 89%) as beige solid. The product was used in the next reaction without any further purification. LCMS m/z Expected 1955.8, Observed 979.4 [M+H]+(z=2).

### Synthesis of Precursor (6)

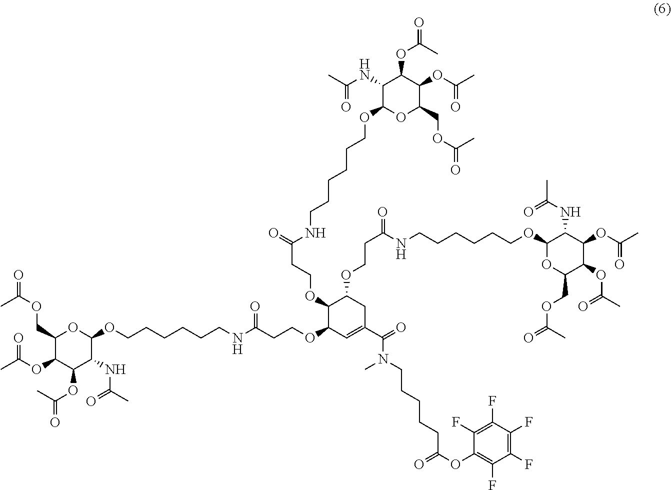 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)N(C)CCCCCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Synthetic Scheme

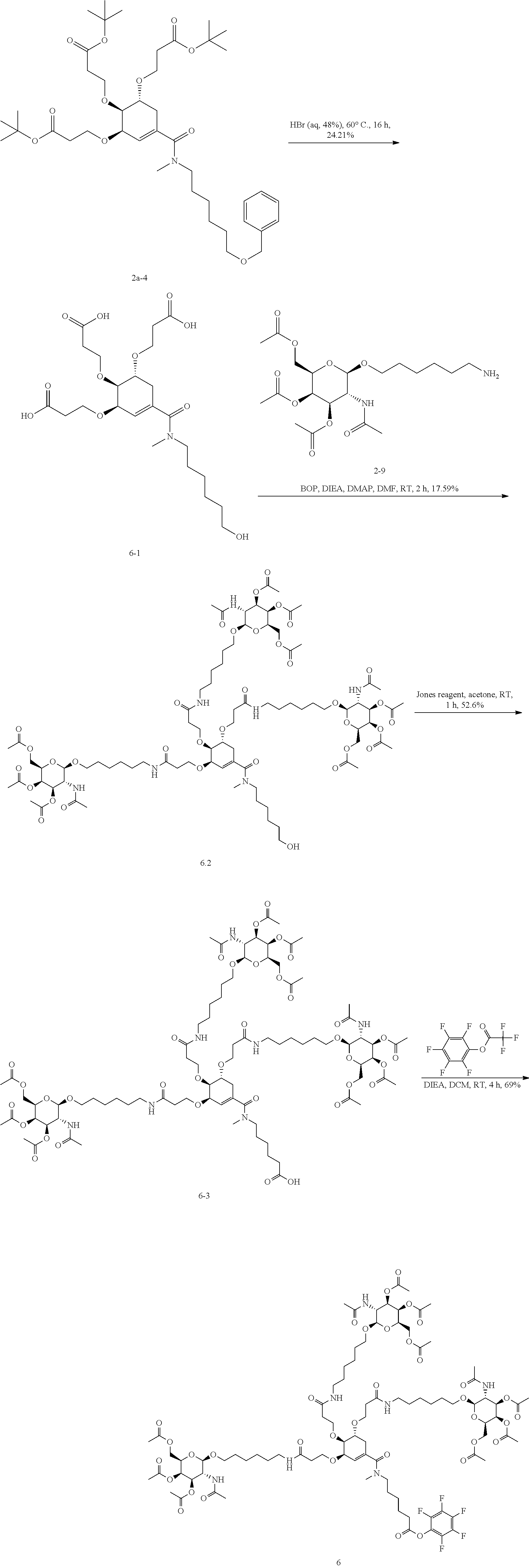

Tri-tert-butyl 3,3′,3″-(((1R,2S,3R)-5-((6-(benzyloxy)hexyl)(methyl)carbamoyl)cyclohex-4-ene-1,2,3-triyl)tris(oxy))tripropionate (2a-4) (1 g, 1.31 mmol) was added in HBr (aq, 48%, 25 mL) at rt. The resulting mixture was stirred at rt for 1 hour. The resulting mixture was stirred at 60° C. for 16 hours. The solvent was removed under reduced pressure. The crude product was purified by flash C18-flash chromatography, elution gradient 0 to 25% MeCN in water (0.1% TFA). Pure fractions were evaporated to dryness to afford 3,3′,3″-(((1R,2S,3R)-5-((6-hydroxyhexyl)(methyl)carbamoyl)cyclohex-4-ene-1,2,3-triyl)tris(oxy))tripropionic acid (6-1) (0.160 g, 24.21%) as a pale yellow gum.

1H-NMR (400 MHz, DMSO, 21° C.) δ 1.08-1.74 (m, 9H), 2.01 (d, 1H), 2.35-2.48 (m, 6H), 2.83 (d, J=40.9 Hz, 3H), 3.25 (s, 2H), 3.38 (t, J=6.5 Hz, 1H), 3.63-3.83 (m, 8H), 4.04 (s, 2H), 4.37 (t, J=6.6 Hz, 1H), 5.50 (s, 1H), 12.19 (brs, 2H). LCMS m/z Expected 503.2, Observed 504.3 ([M+H]⁺). 3,3′,3″-(((1R,2S,3R)-5-((6-hydroxyhexyl)(methyl)carbamoyl)cyclohex-4-ene-1,2,3-triyl)tris(oxy))tripropionic acid (6-1) (160 mg, 0.32 mmol) was added to N-ethyl-N-isopropylpropan-2-amine (1.110 mL, 6.35 mmol), ((1H-benzo[d][1,2,3]triazol-1-yl)oxy)tris(dimethylamino)phosphonium hexafluorophosphate(V) (703 mg, 1.59 mmol), (2R,3R,4R,5R,6R)-5-acetamido-2-(acetoxymethyl)-6-((6-aminohexyl)oxy)tetrahydro-2H-pyran-3,4-diyl diacetate (2-9) (709 mg, 1.59 mmol) and DMAP (116 mg, 0.95 mmol) in DMF (8 mL). The resulting mixture was stirred at rt for 2 hours. The solvent was removed under reduced pressure. The crude product was purified by flash C18-flash chromatography, elution gradient 0 to 40% MeCN in water (0.1% FA). Pure fractions were evaporated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[6-hydroxyhexyl(methyl)carbamoyl]cyclohex-3-en-1-yl]oxypropanoylamino]hexoxy]tetrahydropyran-2-yl]methyl acetate (6-2) (100 mg, 17.59%) as a pale yellow solid.

1H NMR (300 MHz, DMSO, 28° C.) δ 1.2-1.46 (m, 32H), 1.77 (s, 9H), 1.90 (s, 8H), 2.00 (s, 11H), 2.11 (s, 9H), 2.22-2.39 (m, 7H), 2.65-2.93 (m, 6H), 2.94-3.08 (m, 6H), 3.6-3.79 (m, 13H), 3.8-3.93 (m, 4H), 4.03 (s, 11H), 4.49 (d, J=8.4 Hz, 3H), 4.97 (dd, J=11.2, 3.4 Hz, 3H), 5.22 (d, J=3.3 Hz, 3H), 5.49 (s, 1H), 7.76-7.94 (m, 5H). LCMS m/z Expected 1787.8, Observed 895.5 ([M+H]⁺) (z=2).

Chromosulfuric acid (0.056 mL, 0.11 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[6-hydroxyhexyl(methyl)carbamoyl]cyclohex-3-en-1-yl]oxypropanoylamino]hexoxy]tetrahydropyran-2-yl]methyl acetate (6-2) (100 mg, 0.06 mmol) in acetone (10 mL) at 0° C. The resulting mixture was stirred at rt for 1 hour. The reaction mixture was quenched with isopropyl alcohol (5 mL) and diluted with DCM (20 mL). The reaction mixture was poured into water (20 mL), extracted with DCM (2×20 mL).

The organic layer was washed with NaHCO₃ (2×20 mL), saturated brine (20 mL), dried over Na2SO4, filtered and evaporated to afford white solid. The crude product was purified by flash C18-flash chromatography, elution gradient 0 to 40% MeCN in water (0.1% FA). Pure fractions were evaporated to dryness to afford 6-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)-N-methylcyclohex-1-ene-1-carboxamido)hexanoic acid (6-3) (53.0 mg, 52.6%) as a white solid.

1H NMR (400 MHz, DMSO, 20° C.) δ 1.24 (s, 17H), 1.32-1.53 (m, 16H), 1.77 (s, 9H), 1.90 (s, 9H), 2.00 (s, 10H), 2.11 (s, 10H), 2.23-2.41 (m, 7H), 2.74-2.9 (m, 3H), 2.96-3.08 (m, 6H), 3.24 (s, 3H), 3.59-3.81 (m, 11H), 3.82-3.92 (m, 3H), 3.98-4.11 (m, 10H), 4.49 (d, J=8.4 Hz, 3H), 4.97 (dd, J=11.2, 3.2 Hz, 3H), 5.22 (d, J=3.3 Hz, 3H), 5.49 (s, 1H), 7.7-8.03 (m, 6H). LCMS m/z Expected 1081.8, Observed 902.3 ([M+H]⁺) (z=2).

Perfluorophenyl 2,2,2-trifluoroacetate (0.025 mL, 0.15 mmol) was added to (6-3) (53 mg, 0.03 mmol) and DIEA (0.051 mL, 0.29 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness. The residue was diluted with MeCN/water (1:1) 5 mL and dried by lyophilization to give (6) (40.0 mg, 69%) as light brown solid. The product was used in the next reaction without any further purification.

### Synthesis of Precursor (7)

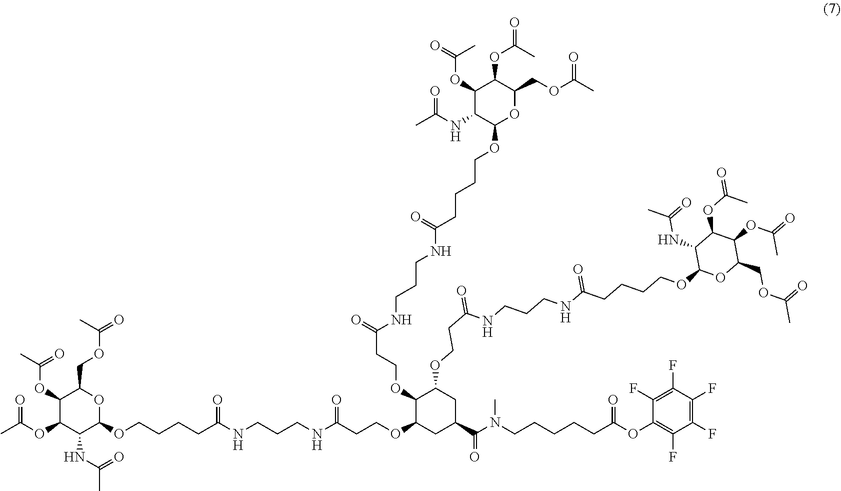 [SMILES: CC(=O)N[C@H]1[C@H](OCCCCC(=O)NCCCNC(=O)CCO[C@@H]2C[C@H](C(=O)N(C)CCCCCC(=O)Oc3c(F)c(F)c(F)c(F)c3F)C[C@@H](OCCC(=O)NCCCNC(=O)CCCCO[C@@H]3O[C@H](COC(C)=O)[C@H](OC(C)=O)[C@H](OC(C)=O)[C@H]3NC(C)=O)[C@@H]2OCCC(=O)NCCCNC(=O)CCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

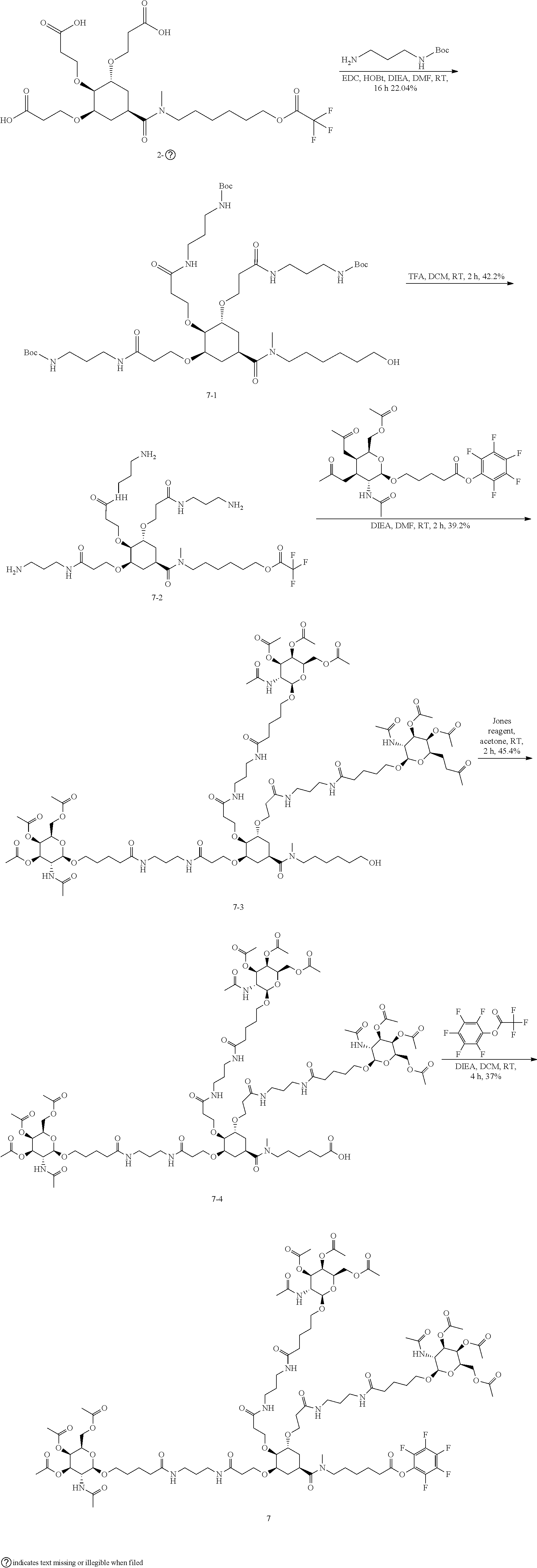

DIEA (2.93 mL, 16.77 mmol) was added to 3,3′,3″-(((1R,2S,3R,5S)-5-(methyl(6-(2,2,2-trifluoroacetoxy)hexyl)carbamoyl)cyclohexane-1,2,3-triyl)tris(oxy))tripropionic acid, Trifluoroacetic acid (1 g, 1.40 mmol), tert-butyl (3-aminopropyl)carbamate (2-6) (1.461 g, 8.38 mmol), EDC (2.68 g, 13.97 mmol) and HOBt (2.140 g, 13.97 mmol) in DMIF (10 mL). The resulting mixture was stirred at rt for 16 hours. The solvent was removed under reduced pressure to afford the crude product. The crude product was purified by flash C18-flash chromatography, elution gradient 0 to 1000% MeCN in water (0.1% NaHCO₃). Pure fractions were evaporated to dryness to afford di-tert-butyl (((3,3′-(((1R,2S,3R,5S)-2-(3-((3-((tert-butoxycarbonyl)amino)propyl)amino)-3-oxopropoxy)-5-((6-hydroxyhexyl)(methyl)carbamoyl)cyclohexane-1,3-diyl)bis(oxy))bis(propanoyl))bis(azanediyl))bis(propane-3,1-diyl))dicarbamate (7-1) (0.300 g, 22.04%) as a yellow oil.

1H NMR (300 MHz, DMSO, 23° C.) δ 1.37 (s, 38H), 1.48 (dd, J=12.7, 6.0 Hz, 11H), 2.08-2.33 (m, 8H), 2.77 (s, 2H), 2.85-2.96 (m, 10H), 2.97-3.11 (m, 8H), 3.5-3.8 (in, 9H). LCMS m/z Expected 973.6, Observed 974.6 ([M+H]).

TFA (3.08 mL, 40.03 mmol) was added to tert-butyl (3-(3-(((1S,2R,4S,6R)-2,6-bis(3-((3-((tert-butoxycarbonyl)amino)propyl)amino)-3-oxopropoxy)-4-((6-hydroxyhexyl)(methyl)carbamoyl)cyclohexyl)oxy)propanamido)propyl)carbamate (7-1) (300 mg, 0.31 mmol) in DCM (3 mL) at 0° C. The resulting mixture was stirred at 0° C. for 2 hours. The solvent was removed under reduced pressure to afford 6-((1S,3R,4S,5R)-3,4,5-tris(3-((3-aminopropyl)amino)-3-oxopropoxy)-N-methylcyclohexane-1-carboxamido)hexyl 2,2,2-trifluoroacetate (7-2) (100 mg, 42.2%) as a yellow oil. The product was used in the next step directly without further purification.

1H NMR (400 MHz, DMSO, 23° C.) δ 1.23 (d, J=7.5 Hz, 2H), 1.41 (d, J=7.2 Hz, 2H), 1.52 (d, J=9.5 Hz, 4H), 1.62-1.74 (m, 10H), 2.31 (dq, J=12.0, 6.4 Hz, 7H), 2.75-2.79 (m, 6H), 2.84-2.9 (m, 3H), 3.09-3.15 (m, 6H), 3.21-3.29 (m, 2H), 3.39 (ddd, J=17.9, 9.4, 4.6 Hz, 2H), 3.55-3.68 (m, 7H), 4.38 (td, J=6.6, 3.6 Hz, 2H), 8.08 (t, J=5.4 Hz, 3H). LCMS m/z Expected 769.4, Observed 770.4 ([M+H]⁺).

DIEA (0.681 mL, 3.90 mmol) was added to 6-((1S,3R,4S,5R)-3,4,5-tris(3-((3-aminopropyl)amino)-3-oxopropoxy)-N-methylcyclohexane-1-carboxamido)hexyl 2,2,2-trifluoroacetate (7-2) (300 mg, 0.39 mmol) and (2R,3R,4R,5R,6R)-5-acetamido-2-(acetoxymethyl)-6-((5-oxo-5-(perfluorophenoxy)pentyl)oxy)tetrahydro-2H-pyran-3,4-diyl diacetate (6-12) (1434 mg, 2.34 mmol) in DMF (5 mL). The resulting mixture was stirred at rt for 2 hours. The solvent was removed under reduced pressure to afford the crude product. The crude product was purified by flash C18-flash chromatography, elution gradient 0 to 100% MeCN in water (0.1% FA). Pure fractions were evaporated to dryness to afford [(2R,3R,4R,5R,6R)-5-acetamido-3,4-diacetoxy-6-[5-[3-[3-[(1R,3R)-2,3-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-5-[6-hydroxyhexyl(methyl)carbamoyl]cyclohexoxy]propanoylamino]propylamino]-5-oxo-pentoxy]tetrahydropyran-2-yl]methyl acetate (7-3) (300 mg, 39.2%) as a colorless oil.

1H NMR (400 MHz, DMSO, 20° C.) δ 1.24 (s, 2H), 1.78 (s, 18H), 1.95 (d, J=42.6 Hz, 46H), 2.28 (p, J=5.6, 5.0 Hz, 13H), 2.95 (s, 3H), 3.02 (s, 2H), 3.54-3.7 (m, 20H), 3.7-3.78 (m, 9H), 4.48 (d, J=8.4 Hz, 9H), 4.97 (dd, J=11.3, 3.4 Hz, 6H), 7.83 (q, J=8.5, 7.0 Hz, 12H), 8.14 (s, 3H). LCMS m/z Expected 1960.9, Observed 1962.3 ([M+H]⁺).

Chromosulfuric acid (0.071 mL, 0.14 mmol) was added to [(2R,3R,4R,5R,6R)-5-acetamido-3,4-diacetoxy-6-[5-[3-[3-[(1R,3R)-2,3-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-5-[6-hydroxyhexyl(methyl)carbamoyl]cyclohexoxy]propanoylamino]propylamino]-5-oxo-pentoxy]tetrahydropyran-2-yl]methyl acetate (7-3) (140 mg, 0.07 mmol) in acetone (5 mL) at 0° C. The resulting mixture was stirred at rt for 2 hours. The solvent was removed under reduced pressure to afford the crude product. The crude product was purified by flash C18-flash chromatography, elution gradient 0 to 100% MeCN in water (0.05% FA). Pure fractions were evaporated to dryness to afford 6-((3R,5R)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)-N-methylcyclohexane-1-carboxamido)hexanoic acid (7-4) (64.0 mg, 45.4%) as a white solid.

1H NMR (500 MHz, DMSO, 24° C.) δ 1.20 (d, J=7.2 Hz, 1H), 1.24 (s, 1H), 1.4-1.59 (m, 27H), 1.78 (s, 9H), 1.90 (s, 9H), 2.00 (s, 9H), 2.05 (t, J=7.0 Hz, 7H), 2.11 (s, 10H), 2.28 (dq, J=13.2, 6.8 Hz, 6H), 2.71 (s, 1H), 2.95 (s, 3H), 2.99-3.08 (m, 12H), 3.40 (t, J=4.4 Hz, 3H), 3.42 (t, J=4.8 Hz, 3H), 3.60 (dt, J=16.0, 5.2 Hz, 5H), 3.71 (tq, J=15.5, 9.7, 7.4 Hz, 6H), 3.84-3.92 (m, 3H), 4.03 (h, J=4.1 Hz, 9H), 4.46-4.55 (m, 3H), 4.98 (dd, J=11.2, 3.5 Hz, 3H), 5.22 (d, J=3.4 Hz, 3H), 7.68-8.05 (m, 8H). LCMS m/z Expected 1974.9, Observed 1976.70 ([M+H]⁺).

Perfluorophenyl 2,2,2-trifluoroacetate (0.026 mL, 0.15 mmol) was added to (7-4) (60 mg, 0.03 mmol) and DIEA (0.053 mL, 0.3 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness. The residue was diluted with MeCN/water (1:1) 5 mL and dried by lyophilization to give (7) (24.0 mg, 37%) as light brown solid. The product was used in the next reaction without any further purification. LCMS m/z Expected 2140.9, Observed 1072.5 [M+H](z=2).

### Synthesis of Precursor (8)

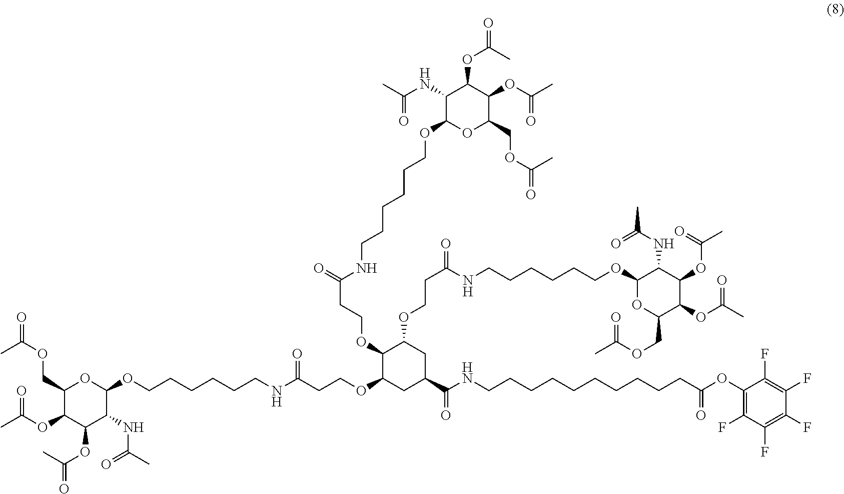

Synthetic scheme:

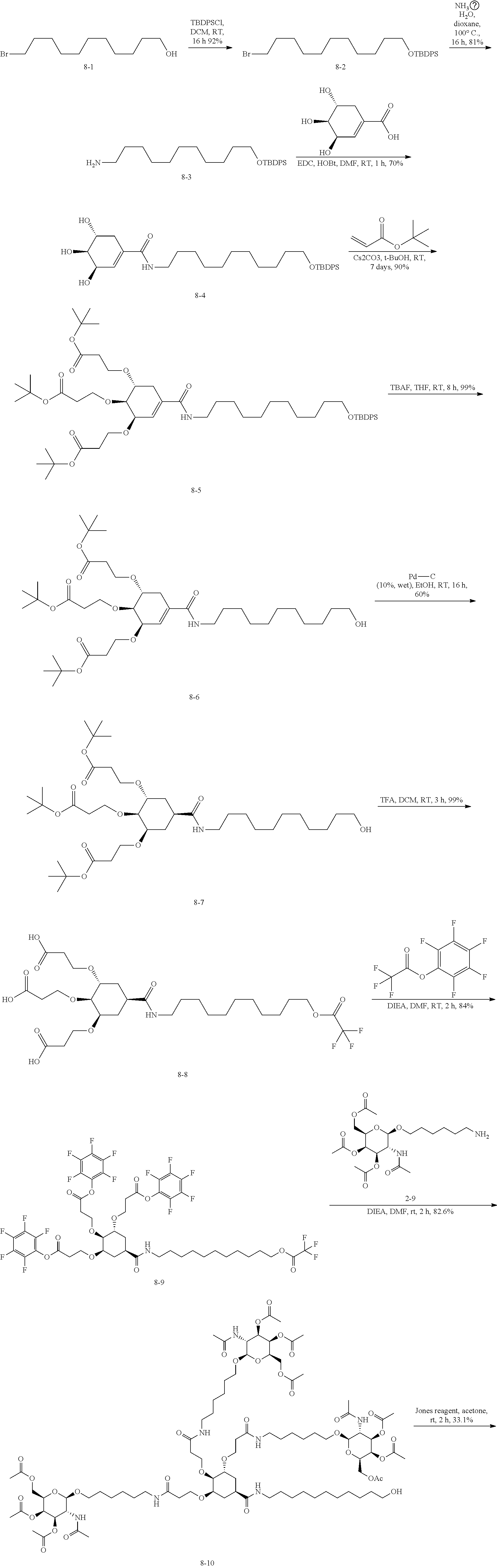

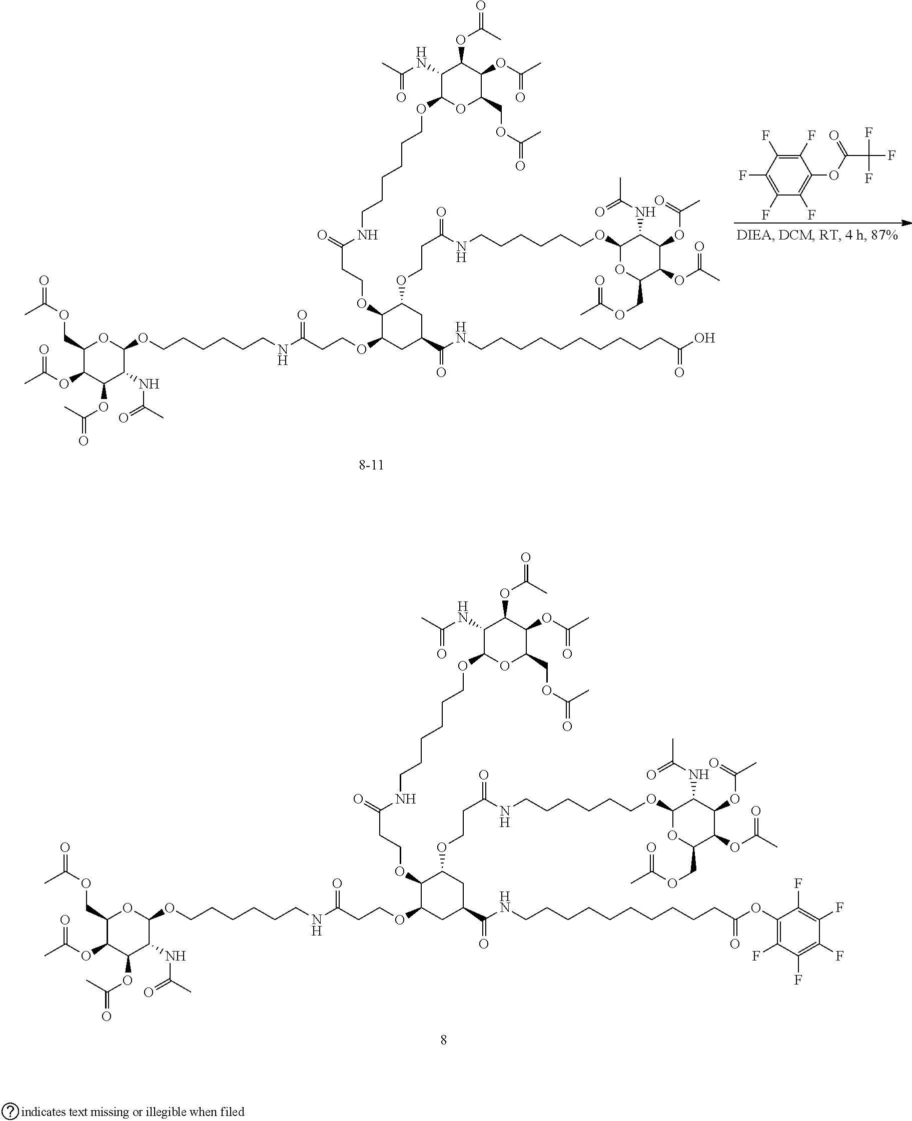

TBDPS-Cl (153 mL, 597.11 mmol) was added slowly to 11-bromoundecan-1-ol (100 g, 398.08 mmol) (8-1) and imidazole (81 g, 1194.23 mmol) in DCM (1500 mL) at 0° C. under nitrogen. The resulting mixture was stirred at rt for 16 hours. The reaction mixture was poured into water (1000 mL), the organic layer was washed sequentially with saturated NaHCO₃ (2×1000 ml), and washed with saturated NaCl (2×1000 ml), the organic layer was dried over Na2SO4, filtered and evaporated to afford ((11-bromoundecyl)oxy)(tert-butyl)diphenylsilane (8-2) (180 g, 92%) as a yellow oil. The product was used in the next step directly without further purification.

1H NMR (300 MHz, DMSO, 22° C.) δ 0.99 (s, 9H), 1.23 (d, J=4.6 Hz, 10H), 1.35 (q, J=6.9, 6.5 Hz, 4H), 1.51 (p, J=6.4 Hz, 2H), 1.77 (p, J=6.8 Hz, 2H), 3.50 (d, J=13.4 Hz, 2H), 3.63 (d, J=12.6 Hz, 2H), 7.38-7.54 (m, 6H), 7.58-7.64 (m, 4H). LCMS m/z Expected 488.2, No mass signal observed.

((11-bromoundecyl)oxy)(tert-butyl)diphenylsilane (8-2) (170 g, 347.21 mmol) in NH3·H2O/dioxane=1:1 (2500 mL) was stirred in high pressure reactor at 100° C. for 16 hours. The solvent was removed under reduced pressure to afford 11-((tert-butyldiphenylsilyl)oxy)undecan-1-amine (8-3) (120 g, 81%) as a yellow oil. The product was used in the next step directly without further purification.

1H NMR (300 MHz, DMSO, 23° C.) δ 0.99 (s, 9H), 1.17-1.37 (m, 14H), 1.49 (dp, J=14.5, 6.8 Hz, 4H), 2.66 (t, J=7.3 Hz, 2H), 3.64 (d, J=12.6 Hz, 2H), 7.4-7.51 (m, 6H), 7.56-7.65 (m, 4H). Two protons exchanged. LCMS m/z Expected 425.3, Observed 426.2. ([M+H]⁺).

11-((tert-butyldiphenylsilyl)oxy)undecan-1-amine (8-3) (55.0 g, 129.20 mmol) was added to (3R,4S,5R)-3,4,5-trihydroxycyclohex-1-ene-1-carboxylic acid (22.5 g, 129.20 mmol), EDC (52.0 g, 271.31 mmol), HOBt (39.6 g, 258.39 mmol) and DIEA (67.7 mL, 387.59 mmol) in DMF (750 mL). The resulting mixture was stirred at RT for 1 hour. The reaction mixture was diluted with H2O (1 L) and evaporated to afford the crude product. The crude product was purified by flash silica chromatography, elution gradient 0 to 10% MeOH in DCM. Pure fractions were evaporated to dryness to afford (3R,4S,5R)—N-(11-((tert-butyldiphenylsilyl)oxy)undecyl)-3,4,5-trihydroxycyclohex-1-ene-1-carboxamide (8-4) (53 g, 70%) as a yellow oil.

1H NMR (300 MHz, DMSO, 20° C.) δ 0.99 (s, 9H), 1.32 (s, 8H), 1.37-1.64 (m, 6H), 1.98 (dd, J=17.7, 4.7 Hz, 1H), 2.46 (s, 1H), 2.78-2.87 (m, 1H), 2.97-3.14 (m, 2H), 3.50 (d, J=5.9 Hz, 2H), 3.64 (t, J=6.3 Hz, 3H), 3.82 (d, J=5.9 Hz, 1H), 4.17 (s, 1H), 4.51 (s, 1H), 4.70 (d, J=27.6 Hz, 2H), 6.56 (s, 1H), 7.43-7.46 (m, 5H), 7.59-7.63 (m, 5H), 7.74-7.84 (m, 1H). LCMS m/z Expected 581.3, Observed 582.4 ([M+H]⁺).

Cs₂CO₃ (98 g, 300.75 mmol) was added to (3R,4S,5R)—N-(11-((tert-butyldiphenylsilyl)oxy)undecyl)-3,4,5-trihydroxycyclohex-1-ene-1-carboxamide (50 g, 85.93 mmol) and tert-butyl acrylate (8-4) (755 mL, 5155.80 mmol) in t-BuOH (1000 mL). The resulting mixture was stirred at rt for 7 days. The reaction mixture was diluted with EtOAc (2500 ml). The solution was filtered through celite. The solvent was removed under reduced pressure to afford tri-tert-butyl 3,3′,3″-(((1R,2S,3R)-5-((11-((tert-butyldiphenylsilyl)oxy)undecyl)carbamoyl)cyclohex-4-ene-1,2,3-triyl)tris(oxy))tripropionate (8-5) (75 g, 90%) as a yellow oil. The product was used in the next step directly without further purification.

1H NMR (400 MHz, DMSO, 21° C.) δ 0.95 (s, 4H), 0.98 (d, J=3.4 Hz, 9H), 1.22 (s, 12H), 1.28-1.56 (m, 34H), 2.13-2.46 (m, 6H), 3.06 (d, J=6.4 Hz, 1H), 3.20 (s, 1H), 3.57-3.81 (m, 7H), 4.03 (q, J=7.1 Hz, 1H), 5.84 (dd, J=10.2, 1.8 Hz, 1H), 7.38-7.48 (m, 6H), 7.58-7.63 (m, 4H), 7.85 (q, J=7.2, 6.4 Hz, 1H). LCMS m/z Expected 965.6, Observed 988.8 ([M+Na]⁺).

TBAF in THE (76 mL, 76.06 mmol) was added to tri-tert-butyl 3,3′,3″-(((1R,2S,3R)-5-((11-((tert-butyldiphenylsilyl)oxy)undecyl)carbamoyl)cyclohex-4-ene-1,2,3-triyl)tris(oxy))tripropionate (8-5) (70 g, 72.44 mmol) in THE (1500 mL). The resulting mixture was stirred at rt for 6 hours. The solvent was removed under reduced pressure to afford tri-tert-butyl 3,3′,3″-(((1R,2S,3R)-5-((11-hydroxyundecyl)carbamoyl)cyclohex-4-ene-1,2,3-triyl)tris(oxy))tripropionate (8-6) (53 g, 99%) as a yellow oil. The product was used in the next step directly without further purification.

1H NMR (400 MHz, DMSO, 19° C.) δ 0.95 (d, J=6.3 Hz, 18H), 1.18-1.35 (m, 18H), 1.36-1.42 (m, 14H), 1.56 (ddt, J=13.4, 9.4, 4.7 Hz, 5H), 2.36-2.46 (m, 2H), 3.11-3.23 (m, 5H), 3.56-3.63 (m, 2H), 3.63-3.81 (m, 3H), 6.16-6.35 (m, 1H), 7.71 (d, J=2.1 Hz, 1H). LCMS m/z Expected 729.5, Observed 728.5 ([M−H]⁻).

Pd—C(10%, wet) (5.85 g, 5.49 mmol) was added to tri-tert-butyl 3,3′,3″-(((1R,2S,3R)-5-((11-hydroxyundecyl)carbamoyl)cyclohex-4-ene-1,2,3-triyl)tris(oxy))tripropionate (8-6) (50 g, 68.68 mmol) in EtOH (1500 mL) at rt. The resulting mixture was stirred at rt under hydrogen for 16 hours. The mixture was filtered through a celite pad. The solvent was removed under reduced pressure to afford tri-tert-butyl 3,3′,3″-(((1R,2S,3R,5S)-5-((11-hydroxyundecyl)carbamoyl)cyclohexane-1,2,3-triyl)tris(oxy))tripropionate (100 g, crude) as a yellow oil. The crude product was purified by flash silica chromatography, elution gradient 0 to 30% THE in petroleum ether. Pure fractions were evaporated to dryness to afford tri-tert-butyl 3,3′,3″-(((1R,2S,3R,5S)-5-((11-hydroxyundecyl)carbamoyl)cyclohexane-1,2,3-triyl)tris(oxy))tripropionate (8-7) (60.0 g, 60%) as a colorless oil.

1H NMR (400 MHz, DMSO, 18° C.) δ 1.1-1.3 (m, 17H), 1.3-1.47 (m, 29H), 1.49-1.58 (m, 2H), 1.59-1.69 (m, 1H), 1.76 (m, 1H), 2.2-2.33 (m, 1H), 2.34-2.48 (m, 5H), 2.85-3.06 (m, 2H), 3.37 (d, J=5.6 Hz, 4H), 3.64 (m, 6H), 3.78 (dt, J=9.6, 5.6 Hz, 1H), 4.33 (m, 1H), 7.64-7.73 (m, 1H). LCMS m/z Expected 729.5, Observed 730.7 ([M+H]⁺).

TFA (10.55 mL, 136.99 mmol) was added to tri-tert-butyl 3,3′,3″-(((1R,3R)-5-((11-hydroxyundecyl)carbamoyl)cyclohexane-1,2,3-triyl)tris(oxy))tripropionate (8-7) (2 g, 2.74 mmol) in DCM (20 mL) at rt. The resulting mixture was stirred at rt for 3 hours. The solvent was removed under reduced pressure to afford 3,3′,3″-(((1R,2S,3R,5S)-5-((11-(2,2,2-trifluoroacetoxy)undecyl)carbamoyl)cyclohexane-1,2,3-triyl)tris(oxy))tripropionic acid (8-8) (1.780 g, 99%) as a yellow oil. The product was used to the next step directly without further purification.

1H NMR (300 MHz, DMSO, 24° C.) δ 1.02-1.46 (m, 18H), 1.54 (q, J=7.8 Hz, 3H), 1.63-1.8 (m, 2H), 1.99 (s, 2H), 2.2-2.35 (m, 1H), 2.36-2.47 (m, 4H), 2.98 (q, J=6.6 Hz, 2H), 3.39 (ddd, J=12.6, 9.2, 5.2 Hz, 1H), 3.49-3.85 (m, 6H), 4.03 (q, J=7.1 Hz, 1H), 4.37 (t, J=6.6 Hz, 2H), 7.63-7.73 (m, 1H). Three protons were exchanged. LCMS m/z Expected 657.3, Observed 658.1 ([M+H]⁺).

Perfluorophenyl 2,2,2-trifluoroacetate (1.999 mL, 11.63 mmol) was added to 3,3′,3″-(((1R,2S,3R,5S)-5-((11-(2,2,2-trifluoroacetoxy)undecyl)carbamoyl)cyclohexane-1,2,3-triyl)tris(oxy))tripropionic acid (8-8) (1.7 g, 2.58 mmol) and DIEA (2.93 mL, 16.80 mmol) in DMF (25 mL) at rt. The resulting mixture was stirred at rt for 2 hours. The reaction mixture was diluted with EtOAc (20 ml), and washed sequentially with 1 M NaHSO4 (2×25 ml), saturated NaHCO₃ (2×15 ml), and saturated brine (10 ml). The organic layer was dried over Na2SO4, filtered and evaporated to afford tris(perfluorophenyl) 3,3′,3″-(((1R,2S,3R,5S)-5-((11-(2,2,2-trifluoroacetoxy)undecyl)carbamoyl)cyclohexane-1,2,3-triyl)tris(oxy))tripropionate (8-9) (2.500 g, 84%) as a yellow oil. The product was used to the next step directly without further purification.

1H NMR (300 MHz, DMSO, 24° C.) δ 0.81-1 (m, 10H), 1.04 (d, J=1.1 Hz, 2H), 1.09-1.43 (m, 15H), 1.60 (td, J=18.0, 16.2, 10.2 Hz, 5H), 2.92-3.07 (m, 2H), 3.1-3.29 (m, 3H), 3.34-3.91 (m, 3H), 3.92-4.18 (m, 1H), 4.37 (td, J=6.7, 2.1 Hz, 1H), 7.64-7.69 (m, 1H). LCMS m/z Expected 1155.2, Observed 1156.4 ([M+H]⁺).

tris(perfluorophenyl) 3,3′,3″-(((1R,2S,3R,5S)-5-((11-(2,2,2-trifluoroacetoxy)undecyl)carbamoyl)cyclohexane-1,2,3-triyl)tris(oxy))tripropionate (8-9) (1.5 g, 1.30 mmol) in DMF (20.0 mL) was added to (2R,3R,4R,5R,6R)-5-acetamido-2-(acetoxymethyl)-6-((6-aminohexyl)oxy)tetrahydro-2H-pyran-3,4-diyl diacetate (2.086 g, 4.67 mmol) (2-9) and DIEA (2.267 mL, 12.98 mmol) in DMF (50 mL) at rt. The resulting mixture was stirred at rt for 2 hours. The reaction mixture was diluted with water (200 mL), and extracted with DCM (3×200 ml). The organic layer was dried over Na2SO4, filtered and evaporated to afford crude product. The crude product was purified by flash C18-flash chromatography, elution gradient 0 to 50% MeCN in water (0.1% FA). Pure fractions were evaporated to dryness to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(11-hydroxyundecylcarbamoyl)cyclohexoxy]propanoylamino]hexoxy]tetrahydropyran-2-yl]methyl acetate (8-10) (1.500 g, 62.6%) as a white solid.

1H NMR (300 MHz, DMSO, 23° C.) δ 1.24 (s, 27H), 1.42 (d, J=24.4 Hz, 15H), 1.55 (s, 3H), 1.77 (s, 9H), 1.90 (s, 9H), 2.00 (s, 9H), 2.11 (s, 9H), 2.29 (q, J=7.1 Hz, 7H), 3.02 (q, J=7.0 Hz, 8H), 3.28-3.49 (m, 7H), 3.49-3.78 (m, 11H), 3.87 (q, J=9.7, 9.3 Hz, 3H), 3.94-4.1 (m, 9H), 4.32 (t, J=5.1 Hz, 1H), 4.49 (d, J=8.4 Hz, 3H), 4.97 (dd, J=11.3, 3.4 Hz, 3H), 5.22 (d, J=3.4 Hz, 3H), 7.55-7.93 (m, 7H). LCMS m/z Expected 1845.9, Observed 1847.7.

Chromosulfuric acid (0.650 mL, 1.30 mmol) was added to [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(11-hydroxyundecylcarbamoyl)cyclohexoxy]propanoylamino]hexoxy]tetrahydropyran-2-yl]methyl acetate (8-10) (1.2 g, 0.65 mmol) in acetone (30 mL) at 0° C. The resulting mixture was stirred at rt for 2 hours. The reaction mixture was quenched with i-PrOH (25 ml), diluted by DCM (100 ml), and washed sequentially with saturated NaHCO₃ (100 mL), saturated brine (100 mL). The organic layer was dried over Na2SO4, filtered, and evaporated to afford the crude product. The crude product was purified by flash C18-flash chromatography, elution gradient 0 to 30% MeCN in water (0.1% FA). Pure fractions were evaporated to dryness to afford 11-((3R,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohexane-1-carboxamido)undecanoic acid (8-11) (0.400 g, 33.1%) as a white solid.

1H NMR (500 MHz, DMSO, 25° C.) δ 1.24 (d, J=5.1 Hz, 25H), 1.32-1.4 (m, 8H), 1.41-1.62 (m, 12H), 1.77 (s, 9H), 1.90 (s, 9H), 2.00 (s, 9H), 2.11 (s, 9H), 2.19 (t, J=7.4 Hz, 2H), 2.22-2.33 (m, 6H), 2.86-3.1 (m, 8H), 3.41 (dt, J=9.7, 6.5 Hz, 4H), 3.5-3.79 (m, 11H), 3.87 (m, 3H), 3.94-4.09 (m, 9H), 4.49 (dd, J=8.4, 1.8 Hz, 3H), 4.97 (dd, J=11.2, 3.4 Hz, 3H), 5.22 (d, J=3.4 Hz, 3H), 7.69 (dt, J=57.9, 5.6 Hz, 2H), 7.77-7.93 (m, 5H), 11.95 (s, 1H). LCMS m/z Expected 1859.9, Observed 1861.2.

Perfluorophenyl 2,2,2-trifluoroacetate (0.028 mL, 0.16 mmol) was added to (8-11) (100 mg, 0.05 mmol) and DIEA (0.047 mL, 0.27 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness. The residue was diluted with MeCN/water (1:1) 5 mL and dried by lyophilization to give (7) (95.0 mg, 87%) as light brown solid. The product was used in the next reaction without any further purification. LCMS m/z Expected 2025.9, Observed 1014.6 [M+H](z=2).

### Synthesis of Precursor (9)

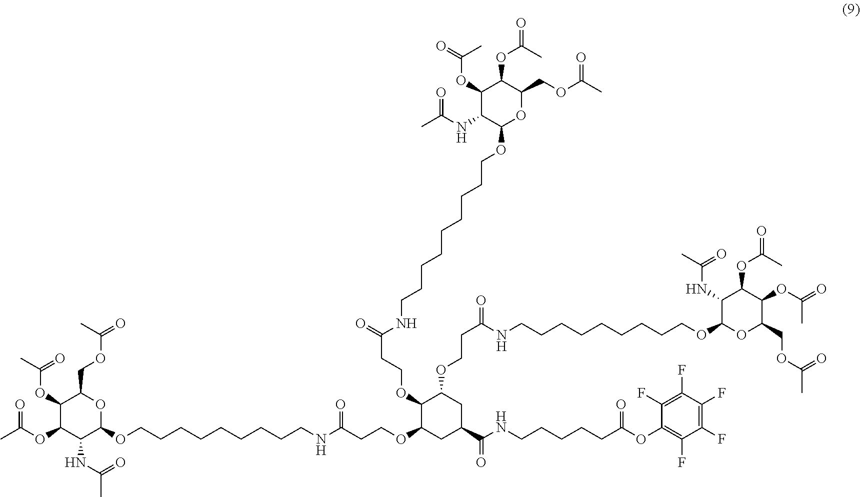 [SMILES: CC(=O)N[C@H]1[C@H](OCCCCCCCCCNC(=O)CCO[C@@H]2C[C@H](C(=O)NCCCCCC(=O)Oc3c(F)c(F)c(F)c(F)c3F)C[C@@H](OCCC(=O)NCCCCCCCCCO[C@@H]3O[C@H](COC(C)=O)[C@H](OC(C)=O)[C@H](OC(C)=O)[C@H]3NC(C)=O)[C@@H]2OCCC(=O)NCCCCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

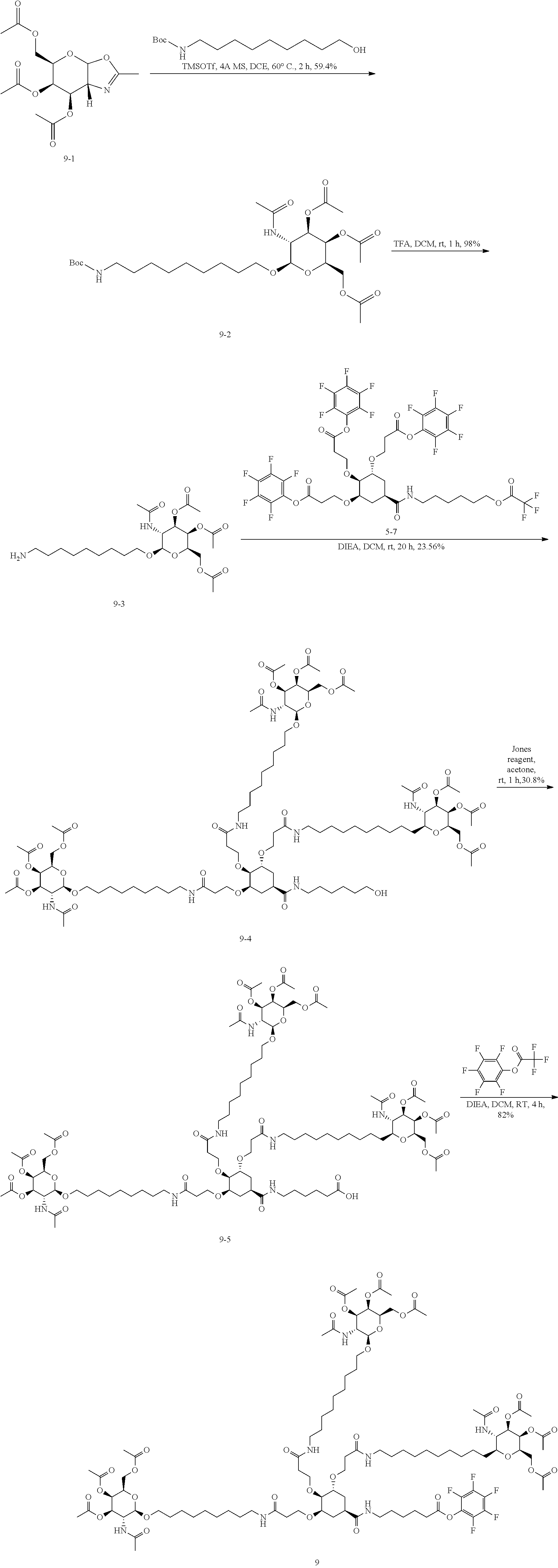

tert-butyl (9-hydroxynonyl)carbamate (6.93 g, 26.72 mmol) was added to (5R,6R,7R,7aR)-5-(acetoxymethyl)-2-methyl-3a,6,7,7a-tetrahydro-5H-pyrano[3,2-d]oxazole-6,7-diyl diacetate (8 g, 24.29 mmol) (9-1) and 4A MS (8 g, 0.00 mmol) in DCE (80 mL) at RT. TMS-OTf (4.39 mL, 24.29 mmol) was added after the reaction had been stirred for 30 minutes at 60° C. The resulting mixture was stirred at 60° C. for 2 hours. The reaction mixture was poured into saturated NaHCO₃ (200 mL), extracted with DCM (2×250 mL), the organic layer was dried over Na2SO4, filtered and evaporated to afford (2R,3R,4R,5R,6R)-5-acetamido-2-(acetoxymethyl)-6-((9-((tert-butoxycarbonyl)amino)nonyl)oxy)tetrahydro-2H-pyran-3,4-diyl diacetate (9-2) (8.50 g, 59.4%) as a brown oil. The product was used in the next step directly without further purification.

1H1 NMR (300 MHz, DMSO, 23° C.) δ 1.21-1.27 (m, 14H), 1.37 (s, 9H), 1.43-1.48 (m, 1H), 1.77 (s, 3H), 1.90 (s, 3H), 2.00 (s, 3H), 2.11 (s, 3H), 2.65 (t, J=7.2 Hz, 1H), 2.88 (q, J=6.5 Hz, 2H), 3.36-3.46 (m, 2H), 3.65-3.77 (m, 1H), 3.81-3.91 (m, 1H), 4.49 (d, J=8.5 Hz, 1H), 4.97 (dd, J=11.2, 3.4 Hz, 1H), 5.22 (d, J=3.4 Hz, 1H), 6.76 (t, J=5.7 Hz, 1H), 7.79-7.88 (in, 1H). LCMS m/z Expected 588.3, Observed 589.3.

TFA (26 mL, 337.48 mmol) was added to (2R,3R,4R,5R,6R)-5-acetamido-2-(acetoxymethyl)-6-((9-((tert-butoxycarbonyl)amino)nonyl)oxy)tetrahydro-2H-pyran-3,4-diyl diacetate (9-3) (6.5 g, 11.04 mmol) in DCM (130 mL) at RT. The resulting mixture was stirred at RT for 1 hour. The solvent was removed under reduced pressure, added DCM (3×50 mL) and evaporated to afford (2R,3R,4R,5R,6R)-5-acetamido-2-(acetoxymethyl)-6-((9-aminononyl)oxy)tetrahydro-2H-pyran-3,4-diyl diacetate (9-3) (5.30 g, 98%) as a brown oil. The product was used in the next step directly without further purification.

1H NMR (300 MHz, DMSO, 23° C.) δ 1.18-1.37 (m, 16H), 1.77 (s, 3H), 1.89 (s, 3H), 2.00 (s, 3H), 2.11 (s, 3H), 3.34-3.47 (m, 1H), 3.66-3.75 (m, 1H), 3.82-3.93 (m, 1H), 4.38 (t, J=6.6 Hz, 1H), 4.49 (d, J=8.5 Hz, 1H), 4.97 (dd, J=11.2, 3.4 Hz, 1H), 5.22 (d, J=3.4 Hz, 1H), 7.73 (s, 4H), 13.78-14.01 (m, 1H). LCMS m/z Expected 488.2, Observed 489.2.

tris(perfluorophenyl) 3,3′,3″-(((1R,2S,3R,5S)-5-((6-(2,2,2-trifluoroacetoxy)hexyl)carbamoyl)cyclohexane-1,2,3-triyl)tris(oxy))tripropionate (2.3 g, 2.12 mmol) (5-7) in DMF (30 mL) was added to (2R,3R,4R,5R,6R)-5-acetamido-2-(acetoxymethyl)-6-((9-aminononyl)oxy)tetrahydro-2H-pyran-3,4-diyl diacetate (9-3) (4.14 g, 8.47 mmol) and DIEA (2.405 mL, 13.77 mmol) in DMF (80 mL) at 0° C. The resulting mixture was stirred at rt for 16 hours. The reaction mixture was diluted with water (200 mL) and extracted with DCM (3×200 ml). The organic layer was dried over Na2SO4, filtered, and evaporated to afford crude product. The crude product was purified by flash C18-flash chromatography, elution gradient 0 to 50% MeCN in water (0.1% FA). Pure fractions were evaporated to dryness to afford [(2R,3R,4R,5R,6R)-5-acetamido-3,4-diacetoxy-6-[9-[3-[(1R,3R)-2,3-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-5-(6-hydroxyhexylcarbamoyl)cyclohexoxy]propanoylamino]nonoxy]tetrahydropyran-2-yl]methyl acetate (9-4) (0.950 g, 23.56%) as a white solid.

1H NMR (300 MHz, DMSO, 23° C.) δ 1.24 (s, 48H), 1.34-1.42 (m, 12H), 1.46 (s, 2H), 1.55 (s, 1H), 1.77 (s, 9H), 1.90 (s, 9H), 2.00 (s, 9H), 2.11 (s, 9H), 2.23-2.32 (m, 7H), 2.94-3.1 (m, 9H), 3.35-3.44 (m, 6H), 3.55-3.63 (m, 1H), 3.67-3.74 (m, 4H), 3.82-3.92 (m, 3H), 4.48 (d, J=8.5 Hz, 3H), 4.93-5.02 (m, 3H), 5.22 (d, J=3.4 Hz, 3H), 7.75 (t, J=5.6 Hz, 1H), 7.78-7.88 (m, 5H). LCMS m/z Expected 1902.0, Observed 1903.3.

Chromosulfuric acid (0.499 mL, 1.00 mmol) was added to [(2R,3R,4R,5R,6R)-5-acetamido-3,4-diacetoxy-6-[9-[3-[(1R,3R)-2,3-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-5-(6-hydroxyhexylcarbamoyl)cyclohexoxy]propanoylamino]nonoxy]tetrahydropyran-2-yl]methyl acetate (9-4) (950 mg, 0.50 mmol) in acetone (15 mL) at 0° C. The resulting mixture was stirred at rt for 1 hour. The reaction mixture was quenched with i-PrOH (15 ml), diluted with DCM (60 ml), and washed sequentially with saturated NaHCO₃ (60 mL), and saturated brine (60 mL). The organic layer was dried over Na2SO4, filtered, and evaporated to afford the crude product. The crude product was purified by flash C18-flash chromatography, elution gradient 0 to 50% MeCN in water (0.1% FA). Pure fractions were evaporated to dryness to afford 6-((3R,5R)-3,4,5-tris(3-((9-(((2R,3R,4R,5R,6R)-3-acetamido-4,5-diacetoxy-6-(acetoxymethyl)tetrahydro-2H-pyran-2-yl)oxy)nonyl)amino)-3-oxopropoxy)cyclohexane-1-carboxamido)hexanoic acid (9-5) (294 mg, 30.8%) as a white solid.

1H NMR (400 MHz, DMSO, 20° C.) δ 1.24 (s, 31H), 1.33-1.62 (m, 22H), 1.77 (s, 9H), 1.90 (s, 9H), 2.00 (s, 9H), 2.11 (s, 9H), 2.16-2.33 (m, 9H), 2.95-3.09 (m, 8H), 3.37-3.44 (m, 3H), 3.54-3.78 (m, 11H), 3.87 (q, J=11.1, 8.8 Hz, 3H), 4.02 (s, 9H), 4.48 (d, J=8.5 Hz, 3H), 4.93-5.01 (m, 3H), 5.22 (d, J=3.4 Hz, 3H), 7.6-7.91 (m, 7H), 12.00 (s, 1H). LCMS m/z Expected 1916.0, Observed 1917.9.

Perfluorophenyl 2,2,2-trifluoroacetate (0.027 mL, 0.16 mmol) was added to (9-5) (100 mg, 0.05 mmol) and DIEA (0.045 mL, 0.26 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness.

The residue was diluted with MeCN/water (1:1) 6 mL and dried by lyophilization to give (9) (89.0 mg, 82%) as light brown solid. The product was used in the next reaction without any further purification. LCMS m/z Expected 2081.9, Observed 1042.4 [M+H]+(z=2).

### Synthesis of Precursor (10)

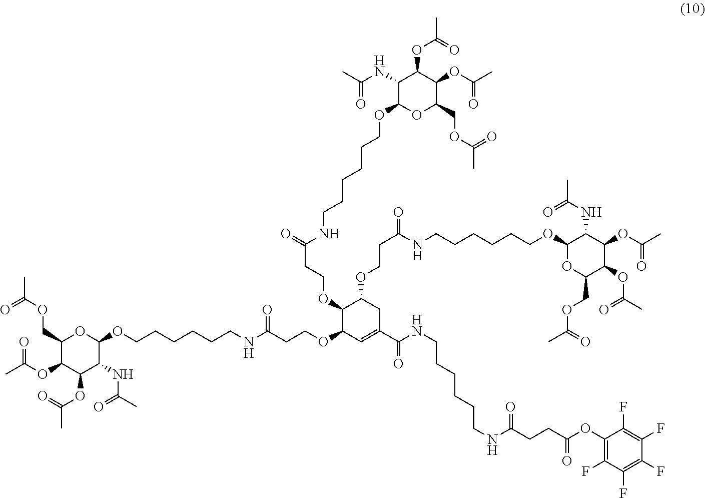 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)NCCCCCCNC(=O)CCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

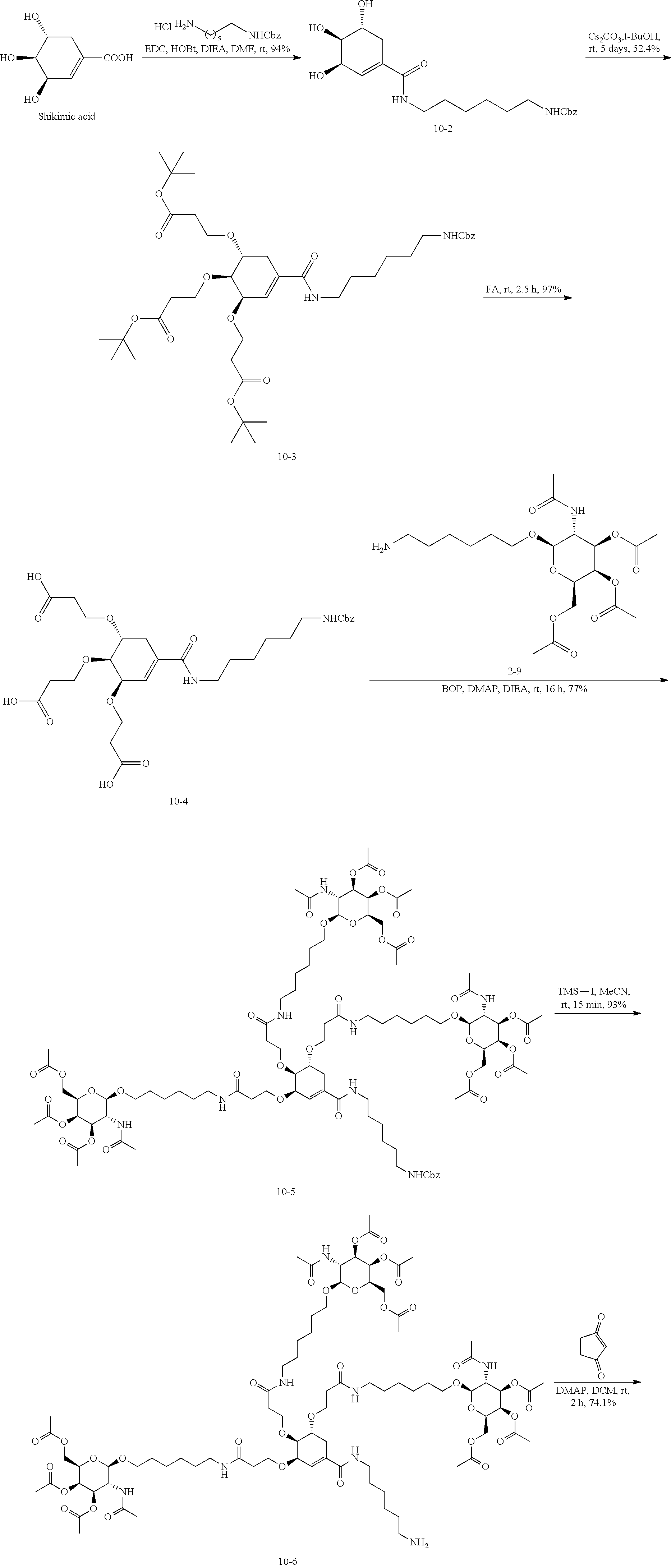

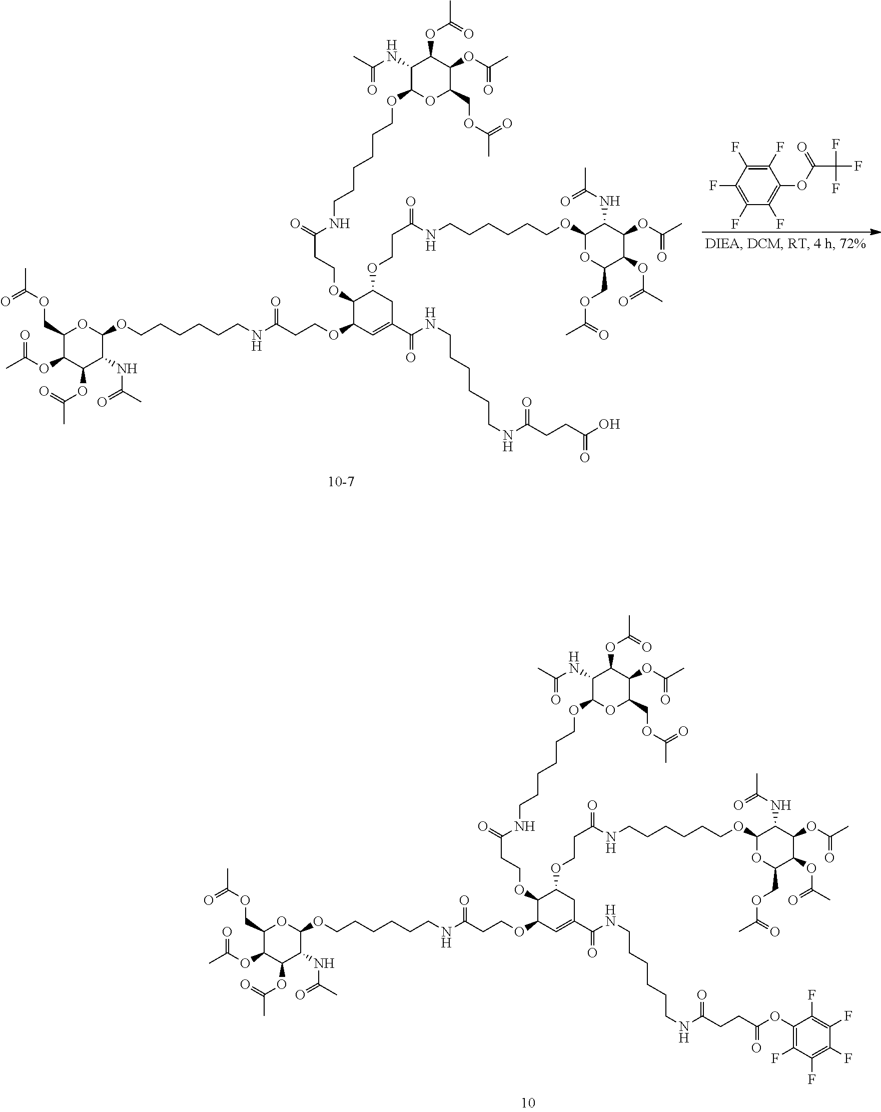

(3R,4S,5R)-3,4,5-trihydroxycyclohex-1-ene-1-carboxylic acid (10.02 g, 57.53 mmol) was added to N-1-Z-1,6-diaminohexane·HCl (15 g, 52.30 mmol), EDC (21.06 g, 109.83 mmol), HOBt (16.02 g, 104.60 mmol) and DIEA (27.4 mL, 156.90 mmol) in DMF (100 mL). The resulting mixture was stirred at rt for 1 hour. The solvent was removed under reduced pressure to afford the crude product. The crude product was purified by flash silica chromatography, elution gradient 0 to 30% MeOH in DCM. Pure fractions were evaporated to dryness to afford benzyl (6-((3R,4S,5R)-3,4,5-trihydroxycyclohex-1-ene-1-carboxamido)hexyl)carbamate (10-2) (20.00 g, 94%) as a white gum.

1H-NMR (500 MHz, DMSO, 25° C.) δ 1.25 (s, 4H), 1.39 (p, J=7.1 Hz, 4H), 1.91-2.04 (m, 1H), 2.98 (q, J=6.6 Hz, 2H), 3.07 (m, 3H), 3.49 (t, J=5.4 Hz, 2H), 3.81 (q, J=5.4 Hz, 1H), 4.17 (s, 1H), 4.51 (s, 1H), 4.66 (s, 1H), 4.75 (s, 1H), 5.01 (s, 2H), 6.27 (dt, J=3.5, 1.8 Hz, 1H), 7.28-7.46 (m, 5H), 7.79 (t, J=5.7 Hz, 1H). LCMS m/z Expected 406.2, Observed 407.2.

Cs₂CO₃ (26.5 g, 81.19 mmol) was added to benzyl (6-((3R,4S,5R)-3,4,5-trihydroxycyclohex-1-ene-1-carboxamido)hexyl)carbamate (10-2) (10 g, 24.60 mmol) and tert-butyl acrylate (216 mL, 1476.09 mmol) in t-BuOH (750 mL) at rt. The resulting mixture was stirred at 40° C. for 5 days. The reaction mixture was filtered through celite. The solvent was removed under reduced pressure to afford the crude product. The crude product was purified by flash silica chromatography, elution gradient 0 to 60% EtOAc in petroleum ether. Pure fractions were evaporated to dryness to afford tri-tert-butyl 3,3′,3″-(((1R,2S,3R)-5-((6-(((benzyloxy)carbonyl)amino)hexyl)carbamoyl)cyclohex-4-ene-1,2,3-triyl)tris(oxy))tripropionate (10-3) (10.20 g, 52.4%) as a colorless oil.

1H-NMR (300 MHz, CDCl3, 22° C.) δ 1.35 (d, J=3.9 Hz, 4H), 1.42-1.58 (m, 31H), 2.33-2.62 (m, 8H), 3.25 (dt, J=21.5, 6.5 Hz, 4H), 3.68-3.97 (m, 8H), 5.11 (s, 2H), 5.91 (s, 1H), 6.30 (s, 1H), 7.31-7.48 (m, 5H). Two protons were exchanged. LCMS m/z Expected 790.4, Observed 791.4.

A solution of tri-tert-butyl 3,3′,3″-(((1R,2S,3R)-5-((6-(((benzyloxy)carbonyl)amino)hexyl)carbamoyl)cyclohex-4-ene-1,2,3-triyl)tris(oxy))tripropionate (10-3) (5 g, 6.32 mmol) in formic acid (75 mL) was stirred at rt for 2.5 hours. The solvent was removed under reduced pressure to afford 3,3′,3″-(((1R,2S,3R)-5-((6-(((benzyloxy)carbonyl)amino)hexyl)carbamoyl)cyclohex-4-ene-1,2,3-triyl)tris(oxy))tripropionic acid (10-4) (3.80 g, 97%) as a colourless oil. The product was used in the next step directly without further purification.

1H-NMR (300 MHz, CDCl3, 22° C.) δ 1.33 (dd, J=20.0, 11.4 Hz, 8H), 2.37 (d, J=17.9 Hz, 1H), 2.5-2.92 (m, 6H), 3.24 (d, J=29.1 Hz, 3H), 3.57-4.31 (m, 8H), 5.13 (d, J=7.6 Hz, 2H), 6.50 (d, J=70.0 Hz, 2H), 7.36 (s, 4H), 8.07 (s, 5H). Three protons were exchanged. LCMS m/z Expected 622.2, Observed 623.2.

(2R,3R,4R,5R)-5-acetamido-2-(acetoxymethyl)-6-((6-aminohexyl)oxy)tetrahydro-2H-pyran-3,4-diyl diacetate (2-9) (10.90 g, 24.41 mmol) in DMF (40 mL) was added to N-ethyl-N-isopropylpropan-2-amine (14.92 mL, 85.44 mmol), ((1H-benzo[d][1,2,3]triazol-1-yl)oxy)tris(dimethylamino)phosphonium hexafluorophosphate(V) (11.88 g, 26.85 mmol),3,3′,3″-(((1R,2S,3R)-5-((6-(((benzyloxy)carbonyl)amino)hexyl)carbamoyl)cyclohex-4-ene-1,2,3-triyl)tris(oxy))tripropionic acid (10-4) (3.8 g, 6.10 mmol) and DMAP (2.237 g, 18.31 mmol) in DMF (60 mL) at rt. The resulting mixture was stirred at rt for 16 hours. The reaction mixture was poured into ice-water (150 mL), extracted with DCM/MeOH=10:1 (5×125 mL), the organic layer was dried over Na2SO4, filtered, and evaporated to afford the crude product. The crude product was purified by flash C18-flash chromatography, elution gradient 0 to 58% MeCN in water (0.1% FA). Pure fractions were evaporated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[6-(benzyloxycarbonylamino)hexylcarbamoyl]cyclohex-3-en-1-yl]oxypropanoylamino]hexoxy]tetrahydropyran-2-yl]methyl acetate (10-5) (9.00 g, 77%) as a white solid.

1H-NMR (300 MHz, CDCl3, 22° C.) δ 1.3-1.61 (m, 32H), 1.93-2.21 (m, 40H), 2.38-2.69 (m, 7H), 3.16-3.25 (m, 7H), 3.58 (d, J=47.8 Hz, 5H), 3.77-3.97 (m, 13H), 3.98-4.3 (m, 11H), 4.68 (d, J=8.4 Hz, 3H), 5.12 (d, J=10.9 Hz, 3H), 5.37 (d, J=3.4 Hz, 3H), 6.38 (s, 1H), 6.76-7.05 (m, 5H), 7.36 (d, J=3.9 Hz, 6H), 7.46-7.56 (m, 1H), 7.77-7.9 (m, 1H). LCMS m/z Expected 1906.9, Observed 1908.1.

TMS-I (2.140 mL, 15.72 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[6-(benzyloxycarbonylamino)hexylcarbamoyl]cyclohex-3-en-1-yl]oxypropanoylamino]hexoxy]tetrahydropyran-2-yl]methyl acetate (10-5) (3 g, 1.57 mmol) in MeCN (30 mL) at. The resulting mixture was stirred at rt for 15 minutes. TEA (2.191 mL, 15.72 mmol) was added to the reaction mixture at 0° C. The mixture was stirred at rt for 15 minutes. The solvent was removed under reduced pressure to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(6-aminohexylcarbamoyl)cyclohex-3-en-1-yl]oxypropanoylamino]hexoxy]tetrahydropyran-2-yl]methyl acetate (10-6) (2.60 g, 93%) as a yellow solid. The product was used in the next step directly without further purification.

1H NMR (400 MHz, CDCl3, 21° C.) δ 1.33 (d, J=13.4 Hz, 10H), 1.49 (d, J=7.3 Hz, 57H), 1.91-2.08 (m, 23H), 2.54 (d, J=31.4 Hz, 3H), 3.35-3.61 (m, 4H), 3.61-4 (m, 7H), 4.0-4.3 (m, 5H), 4.83 (dd, J=12.1, 9.1 Hz, 1H), 5.32 (s, 14H), 6.47-6.68 (m, 1H), 6.93 (dd, J=27.6, 6.9 Hz, 1H), 7.22-7.29 (m, 1H), 7.33-7.48 (m, 2H), 7.48-7.68 (m, 1H), 7.86-8.31 (m, 2H). LCMS m/z Expected 1772.8, Observed 1773.9.

Dihydrofuran-2,5-dione (0.259 g, 2.59 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(6-aminohexylcarbamoyl)cyclohex-3-en-1-yl]oxypropanoylamino]hexoxy]tetrahydropyran-2-yl]methyl acetate (10-6) (2.3 g, 1.30 mmol), TEA (0.904 mL, 6.48 mmol) and DMAP (0.016 g, 0.13 mmol) in EA (40 mL) at. The resulting mixture was stirred at rt for 2 hours. The solvent was removed under reduced pressure to afford the crude product. The crude product was purified by flash C18-flash chromatography, elution gradient 0 to 30% MeCN in water (0.1% FA). Pure fractions were evaporated to dryness to afford 4-oxo-4-((6-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-ene-1-carboxamido)hexyl)amino)butanoic acid (10-7) (1.799 g, 74.1%) as a white solid.

1H-NMR (500 MHz, DMSO, 24° C.) δ 1.03-1.3 (m, 19H), 1.37 (h, J=6.8 Hz, 10H), 1.45 (q, J=6.7 Hz, 6H), 1.78 (s, 9H), 1.90 (s, 9H), 2.00 (s, 10H), 2.11 (s, 10H), 2.25-2.37 (m, 7H), 2.41 (t, J=7.0 Hz, 2H), 3.03 (tt, J=12.2, 6.9 Hz, 9H), 3.41 (dt, J=10.0, 6.6 Hz, 3H), 3.59-3.8 (m, 10H), 3.87 (dt, J=11.1, 8.8 Hz, 3H), 4.03 (h, J=4.2 Hz, 10H), 4.49 (d, J=8.4 Hz, 3H), 4.97 (dd, J=11.2, 3.4 Hz, 3H), 5.22 (d, J=3.4 Hz, 3H), 6.28 (s, 1H), 7.66-7.94 (m, 8H), 11.98 (brs, 1H). LCMS m/z Expected 1872.9, Observed 1874.1.

Perfluorophenyl 2,2,2-trifluoroacetate (0.028 mL, 0.16 mmol) was added to (10-7) (100 mg, 0.05 mmol) and DIEA (0.046 mL, 0.27 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness. The residue was diluted with MeCN/water (1:1) 6 mL and dried by lyophilization to give (10) (78.0 mg, 72%) as a light brown solid. The product was used in the next reaction without any further purification. LCMS m/z Expected 2038.9, Observed 1020.8 [M+H]+(z=2).

### Synthesis of Precursor (11)

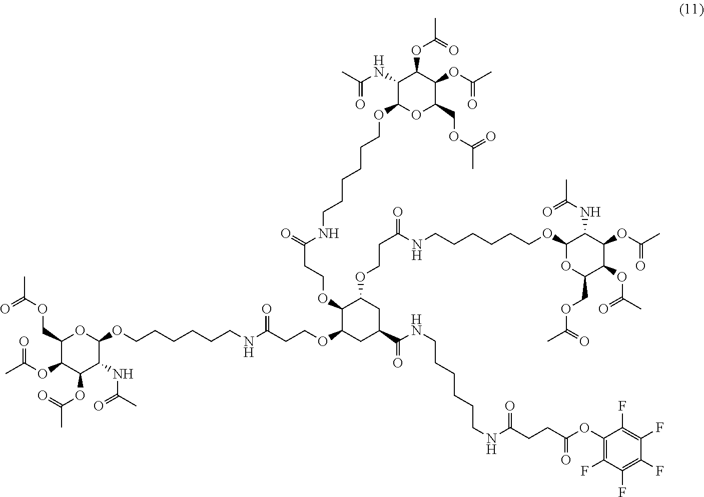 [SMILES: CC(=O)N[C@H]1[C@H](OCCCCCCNC(=O)CCO[C@@H]2C[C@H](C(=O)NCCCCCCNC(=O)CCC(=O)Oc3c(F)c(F)c(F)c(F)c3F)C[C@@H](OCCC(=O)NCCCCCCO[C@@H]3O[C@H](COC(C)=O)[C@H](OC(C)=O)[C@H](OC(C)=O)[C@H]3NC(C)=O)[C@@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Synthetic Scheme:

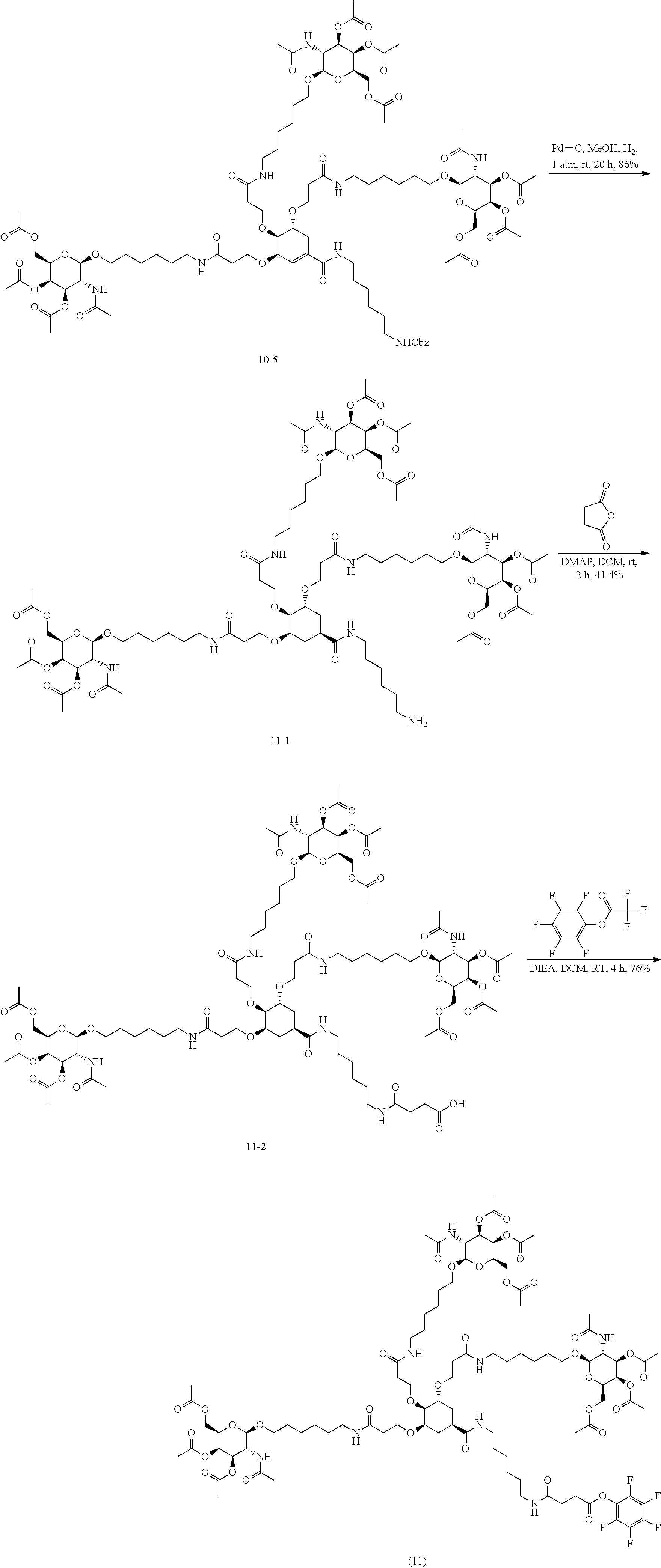

[(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[6-(benzyloxycarbonylamino)hexylcarbamoyl]cyclohex-3-en-1-yl]oxypropanoylamino]hexoxy]tetrahydropyran-2-yl]methyl acetate (10-5) (1.0 g, 0.52 mmol) was dissolved in MeOH (20 mL) and Pd—C(10% wet) (0.056 g, 0.05 mmol) was added, then replaced three times with hydrogen. The reaction mixture was stirred under an atmosphere of hydrogen at 1 atm and rt for 20 hours. The mixture was filtered through a celite pad. The solvent was removed under reduced pressure to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(6-aminohexylcarbamoyl)cyclohexoxy]propanoylamino]hexoxy]tetrahydropyran-2-yl]methyl acetate (11-1) (0.800 g, 86%) as a white solid. The product was used in the next step directly without further purification.

1H NMR (300 MHz, DMSO, 23° C.) δ 1.22 (d, J=20.4 Hz, 16H), 1.41 (d, J=28.1 Hz, 19H), 1.78 (s, 9H), 1.90 (s, 9H), 2.00 (s, 10H), 2.11 (s, 9H), 2.29 (d, J=8.8 Hz, 7H), 2.69-2.83 (m, 2H), 3.02 (d, J=6.0 Hz, 7H), 3.22-3.5 (m, 6H), 3.55-3.78 (m, 10H), 3.8-3.95 (m, 3H), 4.03 (s, 9H), 4.49 (d, J=8.4 Hz, 3H), 4.97 (dd, J=11.2, 3.4 Hz, 3H), 5.22 (d, J=3.4 Hz, 3H), 7.69-7.97 (m, 7H). LCMS m/z Expected 1775.9, Observed 1776.7.

DMAP (0.144 g, 1.18 mmol) was added to [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-(6-aminohexylcarbamoyl)cyclohexoxy]propanoylamino]hexoxy]tetrahydropyran-2-yl]methyl acetate (11-1) (0.7 g, 0.39 mmol) and dihydrofuran-2,5-dione (0.079 g, 0.79 mmol) in DCM (20 mL) at. The resulting mixture was stirred at rt for 2 hours. The solvent was removed under reduced pressure to afford the crude product. The crude product was purified by flash C18-flash chromatography, elution gradient 0 to 30% MeCN in water (0.1% FA). Pure fractions were evaporated to dryness to afford 4-oxo-4-((6-((3R,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohexane-1-carboxamido)hexyl)amino)butanoic acid (11-2) (0.306 g, 41.4%) as a white solid.

1H-NMR (300 MHz, DMSO, 23° C.) δ 1.25 (s, 18H), 1.41 (d, J=28.5 Hz, 20H), 1.78 (s, 9H), 1.90 (s, 9H), 2.00 (s, 9H), 2.11 (s, 8H), 2.27 (q, J=5.5, 3.9 Hz, 10H), 2.88-3.14 (m, 10H), 3.42 (dd, J=10.2, 6.2 Hz, 5H), 3.66 (m, 11H), 3.88 (dt, J=11.1, 8.8 Hz, 3H), 4.03 (d, J=3.7 Hz, 8H), 4.51 (dd, J=8.4, 2.3 Hz, 3H), 4.98 (dt, J=11.2, 2.5 Hz, 3H), 5.22 (d, J=3.4 Hz, 3H), 7.49-8.04 (m, 7H), 8.19 (s, 1H). One proton was exchanged. LCMS m/z Expected 1874.9, Observed 1876.2.

Perfluorophenyl 2,2,2-trifluoroacetate (0.037 mL, 0.21 mmol) was added to (11-2) (100 mg, 0.05 mmol) and DIEA (0.056 mL, 0.32 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (50 mL). The organic layer was dried over Na₂SO₄, filtered and evaporated to dryness. The residue was diluted with MeCN/water (1:1) 6 mL and dried by lyophilization to give (11) (83.0 mg, 76%) as a light brown solid. The product was used in the next reaction without any further purification. LCMS m/z Expected 2040.9, Observed 1022.0 [M+H](z=2).

### Synthesis of Precursor (12)

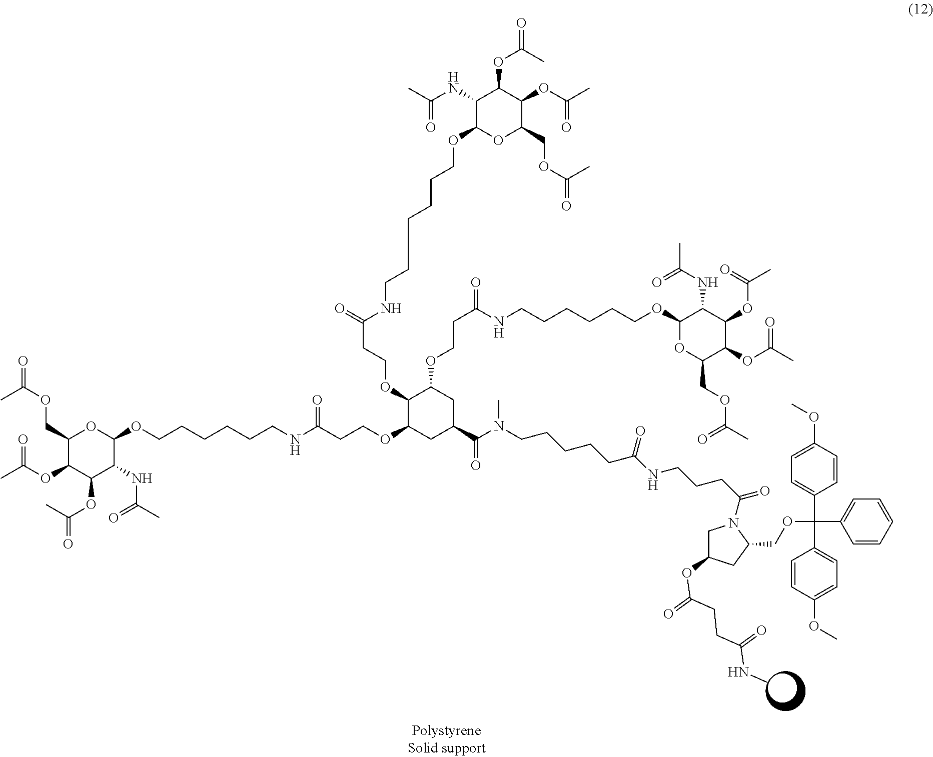 [SMILES: CNC(=O)CCC(=O)O[C@@H]1C[C@@H](COC(c2ccccc2)(c2ccc(OC)cc2)c2ccc(OC)cc2)N(C(=O)CCCNC(=O)CCCCCN(C)C(=O)[C@H]2C[C@@H](OCCC(=O)NCCCCCCO[C@@H]3O[C@H](COC(C)=O)[C@H](OC(C)=O)[C@H](OC(C)=O)[C@H]3NC(C)=O)[C@@H](OCCC(=O)NCCCCCCO[C@@H]3O[C@H](COC(C)=O)[C@H](OC(C)=O)[C@H](OC(C)=O)[C@H]3NC(C)=O)[C@H](OCCC(=O)NCCCCCCO[C@@H]3O[C@H](COC(C)=O)[C@H](OC(C)=O)[C@H](OC(C)=O)[C@H]3NC(C)=O)C2)C1]

### Synthetic Scheme:

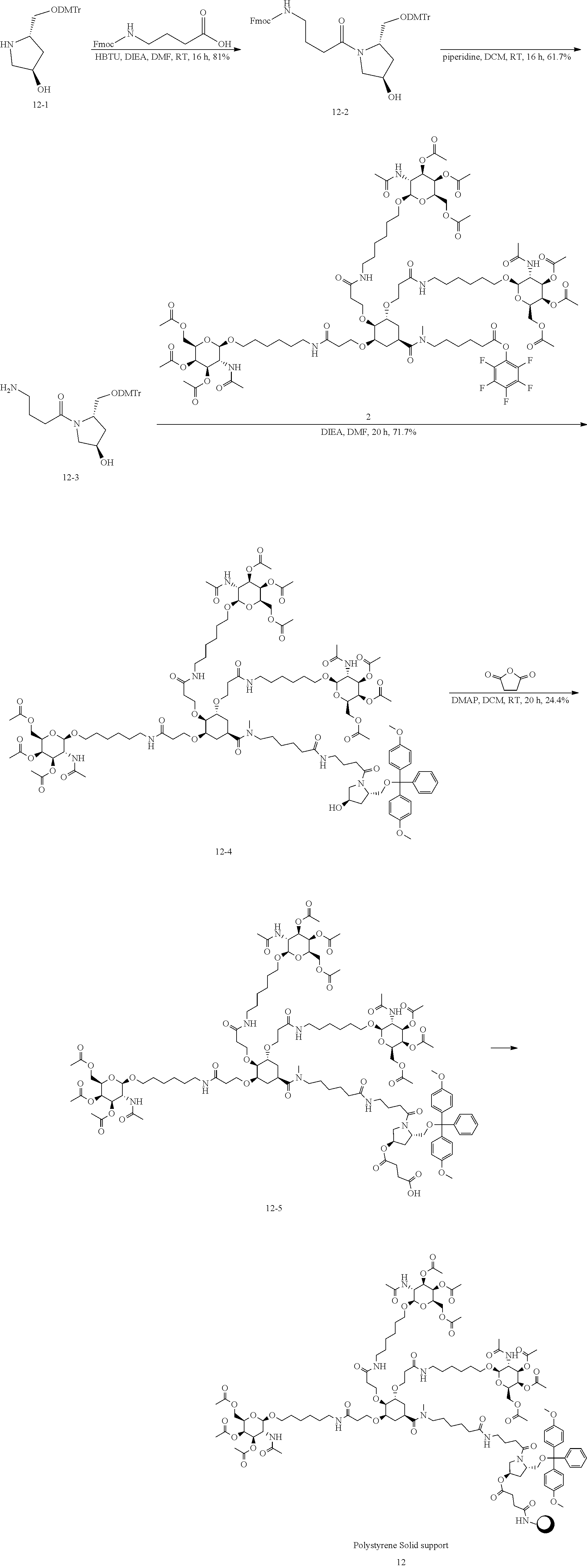

DIEA (9.37 mL, 53.63 mmol) was added to (3R,5S)-5-((bis(4-methoxyphenyl)(phenyl)methoxy)methyl)pyrrolidin-3-ol (5 g, 11.92 mmol), 4-((((9H-fluoren-9-yl) methoxy)carbonyl)amino)butanoic acid (4.65 g, 14.30 mmol) and O-(Benzotriazol-1-yl)-N—N—N′,N′-tetramethyluronium hexafluorophosphate (5.42 g, 14.30 mmol) in DCM (100 mL) at rt under nitrogen. The resulting mixture was stirred at rt for 16 hours. The reaction mixture was poured into water (200 mL), extracted with DCM (3×150 ml), the organic layer was dried over Na2SO4, filtered and evaporated to afford (9H-fluoren-9-yl)methyl (4-((2S,4R)-2-((bis(4-methoxyphenyl)(phenyl)methoxy)methyl)-4-hydroxypyrrolidin-1-yl)-4-oxobutyl) carbamate (12-2) (7.00 g, 81%) as a yellow solid. The product was used in the next step directly without further purification.

1H NMR (400 MHz, DMSO, 22° C.) δ 1.63 (s, 2H), 1.79-2.11 (m, 2H), 2.25 (hept, J=7.1 Hz, 2H), 3.1-3.38 (m, 3H), 3.72 (t, J=2.1 Hz, 6H), 4.07-4.24 (m, 2H), 4.25-4.43 (m, 3H), 6.79-6.92 (m, 4H), 7.13-7.26 (m, 7H), 7.26-7.35 (m, 7H), 7.40 (t, J=7.5 Hz, 2H), 7.44-7.52 (m, 1H), 7.68 (d, J=7.3 Hz, 2H), 7.76 (dt, J=7.8, 1.0 Hz, 1H), 7.88 (dd, J=7.6, 4.6 Hz, 2H). Two protons were exchanged. LCMS m/z Expected 726.3, Observed 727.5 ([M+H]⁺).

Piperidine (49.6 mL, 500.78 mmol) was added to (9H-fluoren-9-yl)methyl (4-((2S,4R)-2-((bis(4-methoxyphenyl)(phenyl)methoxy)methyl)-4-hydroxypyrrolidin-1-yl)-4-oxobutyl)carbamate (12-2) (7 g, 9.63 mmol) in DCM (50 mL) at rt under nitrogen. The resulting mixture was stirred at rt for 16 hours. The solvent was removed under reduced pressure to afford the crude product. The crude product was purified by flash silica chromatography, elution gradient 0 to 30% MeOH in DCM (0.1% TEA). The product was then re-purified by flash C18-flash chromatography, elution gradient 0 to 56% MeCN in water (0.1% NH4HCO3). Pure fractions were evaporated to dryness to afford 4-amino-1-((2S,4R)-2-((bis(4-methoxyphenyl)(phenyl)methoxy)methyl)-4-hydroxypyrrolidin-1-yl)butan-1-one (12-3) (3.00 g, 61.7%) as a white solid.

1H NMR (300 MHz, DMSO, 23° C.) δ 1.63 (dp, J=14.7, 7.8 Hz, 2H), 1.85 (ddd, J=12.9, 8.3, 4.7 Hz, 1H), 1.95-2.12 (m, 1H), 2.19-2.39 (m, 2H), 2.55 (d, J=6.4 Hz, 1H), 2.66 (t, J=7.0 Hz, 1H), 2.99 (dd, J=8.9, 3.2 Hz, 1H), 3.11-3.29 (m, 1H), 3.42 (ddd, J=39.4, 11.3, 4.3 Hz, 1H), 3.59 (dd, J=10.7, 5.1 Hz, 1H), 3.74 (s, 6H), 4.08-4.23 (m, 1H), 4.24-4.48 (m, 2H), 6.89 (ddd, J=9.0, 3.6, 1.1 Hz, 4H), 7.21 (ddd, J=8.8, 6.3, 2.1 Hz, 5H), 7.27-7.39 (m, 4H). Two protons were exchanged. LCMS m/z Expected 504.2, Observed 505.4 ([M+H]⁺).

4-amino-1-((2S,4R)-2-((bis(4-methoxyphenyl)(phenyl)methoxy)methyl)-4-hydroxypyrrolidin-1-yl)butan-1-one (12-3) (4.61 g, 9.13 mmol) was added to (2,3,4,5,6-pentafluorophenyl) 6-[methyl-[(3R,5R)-3,4,5-tris[3-[6-[(2R,3R,4R,5R,6R)-3-acetamido-4,5-diacetoxy-6-(acetoxymethyl)tetrahydropyran-2-yl]oxyhexanoylamino]propoxy]cyclohexanecarbonyl]amino]hexanoate (2) (12 g, 6.09 mmol) and DIEA (5.32 mL, 30.44 mmol) in DMF (240 mL) at rt. The resulting mixture was stirred at rt for 20 hours. The reaction mixture was diluted with H₂O (300 ml). The mixture was extracted with IPA/CHCl3=3:1 (5×500 mL). The organic layer was washed with saturated brine (750 mL). The organic layer was dried over Na2SO4, filtered, and evaporated to afford crude product. The crude product was purified by flash silica chromatography, elution gradient 0 to 20% MeOH in DCM (0.1% TEA), then re-purified by flash C18-flash chromatography, elution gradient 0 to 60% MeCN in water (0.1% NH₄HCO₃). Pure fractions were evaporated to dryness to afford [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-[[6-[[4-[(2S,4R)-2-[[bis(4-methoxyphenyl)-phenyl-methoxy]methyl]-4-hydroxy-pyrrolidin-1-yl]-4-oxo-butyl]amino]-6-oxo-hexyl]-methyl-carbamoyl]cyclohexoxy]propanoylamino]hexoxy]tetrahydropyran-2-yl]methyl acetate (12-4) (10.00 g, 71.7%) as a yellow gum.

1H NMR (300 MHz, DMSO, 24° C.) δ 1.25 (s, 15H), 1.37 (s, 7H), 1.46 (s, 10H), 1.77 (s, 9H), 1.90 (s, 8H), 2.00 (s, 10H), 2.09 (d, J=7.6 Hz, 14H), 2.27 (s, 8H), 2.98 (dd, J=23.1, 6.5 Hz, 13H), 3.59 (s, 8H), 3.74 (s, 18H), 3.88 (q, J=9.5 Hz, 4H), 4.03 (s, 8H), 4.15 (s, 3H), 4.25-4.56 (m, 6H), 4.97 (dd, J=11.3, 3.4 Hz, 4H), 5.22 (d, J=3.4 Hz, 3H), 6.88-6.91 (m, 4H), 7.20 (t, J=2.1 Hz, 5H), 7.3-7.32 (m, 4H), 7.83 (d, J=9.0 Hz, 6H). LCMS m/z Expected 2290.1, Observed 1145.1 ([M−H]-) (z=2).

DMAP (0.376 g, 3.08 mmol) was added to succinic anhydride (0.205 g, 2.05 mmol) and [(2R,3R,4R,5R,6R)-5-acetamido-3,4-diacetoxy-6-[6-[3-[(1R,3R)-2,3-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-5-[[6-[[4-[(2S,4R)-2-[[bis(4-methoxyphenyl)-phenyl-methoxy]methyl]-4-hydroxy-pyrrolidin-1-yl]-4-oxo-butyl]amino]-6-oxo-hexyl]-methyl-carbamoyl]cyclohexoxy]propanoylamino]hexoxy]tetrahydropyran-2-yl]methyl acetate (12-4) (2.35 g, 1.03 mmol) in DCM (30 mL) at 0° C. The resulting mixture was stirred at rt for 20 hours. The solvent was removed under reduced pressure to afford the crude product. The crude product was purified by flash silica chromatography, elution gradient 0 to 25% MeOH in DCM (0.1% TEA), then re-purified by flash C18-flash chromatography, elution gradient 0 to 45% MeCN (0.1% TEA) in water (0.2% TEA). Pure fractions were evaporated to dryness to afford the product. The product was dissolved in MeCN/water (v/v=1:10, 20 ml) and dried by lyophilization to afford 4-(((3R,5S)-5-((bis(4-methoxyphenyl)(phenyl)methoxy)methyl)-1-(4-(6-((3R,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)-N-methylcyclohexane-1-carboxamido)hexanamido)butanoyl)pyrrolidin-3-yl)oxy)-4-oxobutanoic acid (12-5) (0.598 g, 24.40%) as a white solid.

1H NMR (300 MHz, DMSO, 23° C.) δ 0.87-1.29 (m, 15H), 1.31-1.68 (m, 22H), 1.77 (s, 9H), 1.90 (s, 9H), 2.00 (s, 10H), 2.11 (s, 10H), 2.19-2.35 (m, 8H), 2.48 (d, J=6.3 Hz, 4H), 2.69-2.81 (m, 2H), 2.93 (d, J=3.8 Hz, 2H), 3.02 (p, J=6.4, 6.0 Hz, 9H), 3.22 (d, J=5.5 Hz, 3H), 3.40 (dt, J=9.5, 6.1 Hz, 5H), 3.58 (q, J=12.1, 9.2 Hz, 5H), 3.63-3.72 (m, 6H), 3.74 (s, 8H), 3.87 (dt, J=11.3, 8.9 Hz, 3H), 4.03 (s, 9H), 4.20 (s, 1H), 4.49 (d, J=8.4 Hz, 3H), 4.97 (dd, J=11.2, 3.4 Hz, 3H), 5.22 (d, J=3.4 Hz, 3H), 5.38 (s, 1H), 6.88 (dt, J=8.9, 2.3 Hz, 4H), 7.21 (ddt, J=8.9, 4.6, 2.3 Hz, 5H), 7.25-7.39 (m, 4H), 7.66-7.97 (m, 7H), 12.25 (brs, 1H). LCMS m/z Expected 2390.1, Observed 1194.8 ([M−H]-) (z=2).

Synthesis of 12 on polystyrene solid support:

200 mg of amino derivatised polystyrene solid support was added to a 4 mL glass vial. 1.2 mL of acetonitrile was added. The vial was shaken to make a homogenous suspension. HBTU (112 mg, 4 equiv.) and succinate substrate 12-5 (177 mg, 1 equiv.) were added and the vial was shaken for 10 minutes. N-ethyl diisopropylamine (0.08 mL, 6 equiv.) was added to reaction mixture and the shaking was continued overnight. The support was filtered and washed with 2×5 mL acetonitrile. The support was dried under vacuum and transferred to another 4 mL glass vial. The solid support was further treated with capping reagent (2.4 mL) to cap unreacted amine and shaken for 3 hours (capping reagent was prepared by mixing 1.2 mL Capping reagent A with 0.6 mL Capping reagent B1 and 0.6 mL Capping reagent B2—all from Sigma-Aldrich, Novabiochem®). The support was filtered and washed with 5 mL acetonitrile, 5 mL ethanol and again with 5 mL of acetonitrile. The support was dried under vacuum overnight at RT (290 mg, 109.5 micromoles/g loading).

### Synthesis of Precursor (13)

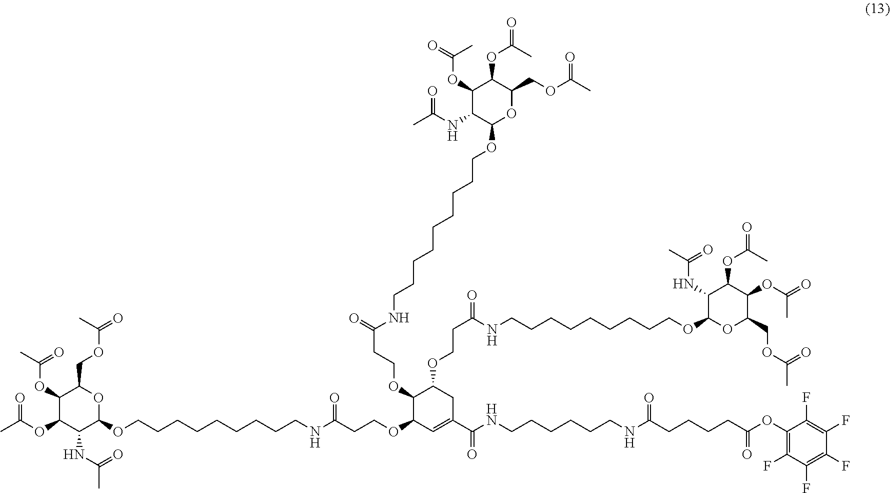 [SMILES: CC(=O)N[C@H]1[C@H](OCCCCCCCCCNC(=O)CCO[C@@H]2[C@H](OCCC(=O)NCCCCCCCCCO[C@@H]3O[C@H](COC(C)=O)[C@H](OC(C)=O)[C@H](OC(C)=O)[C@H]3NC(C)=O)C=C(C(=O)NCCCCCCNC(=O)CCCCC(=O)Oc3c(F)c(F)c(F)c(F)c3F)C[C@H]2OCCC(=O)NCCCCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Synthetic Scheme:

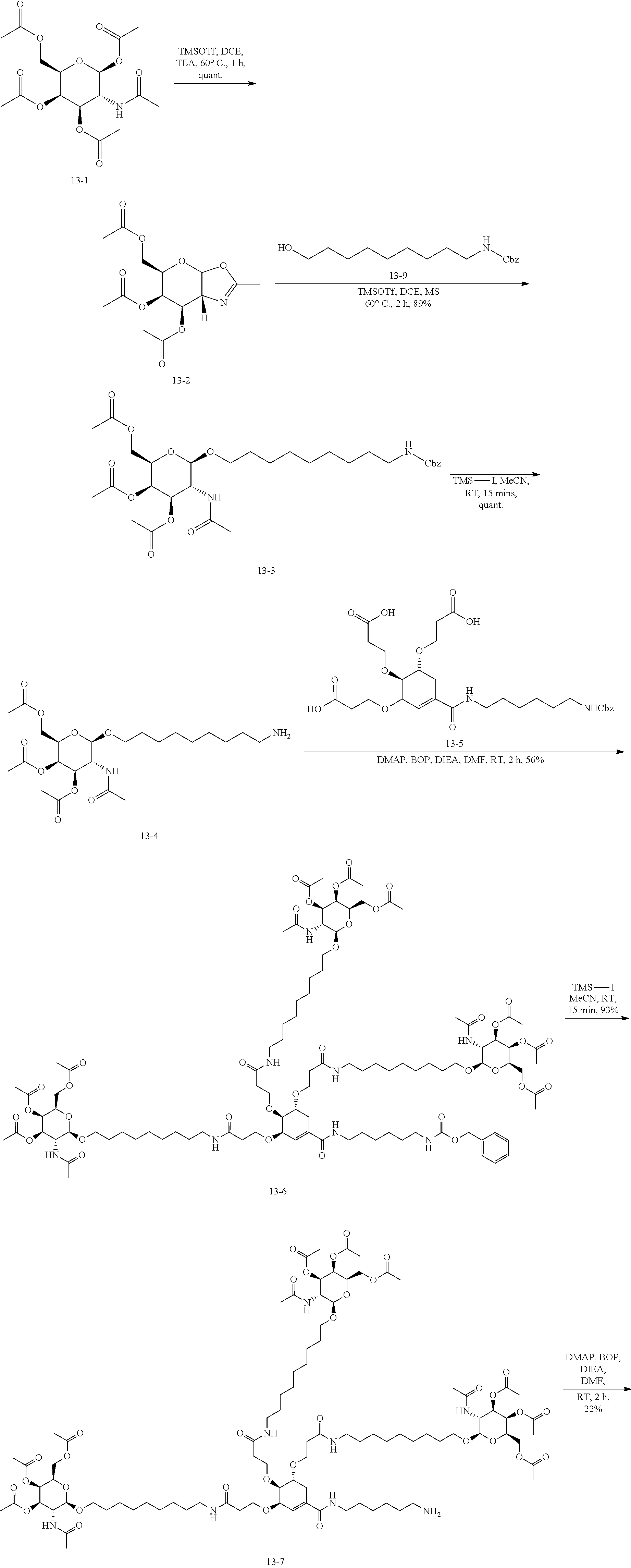

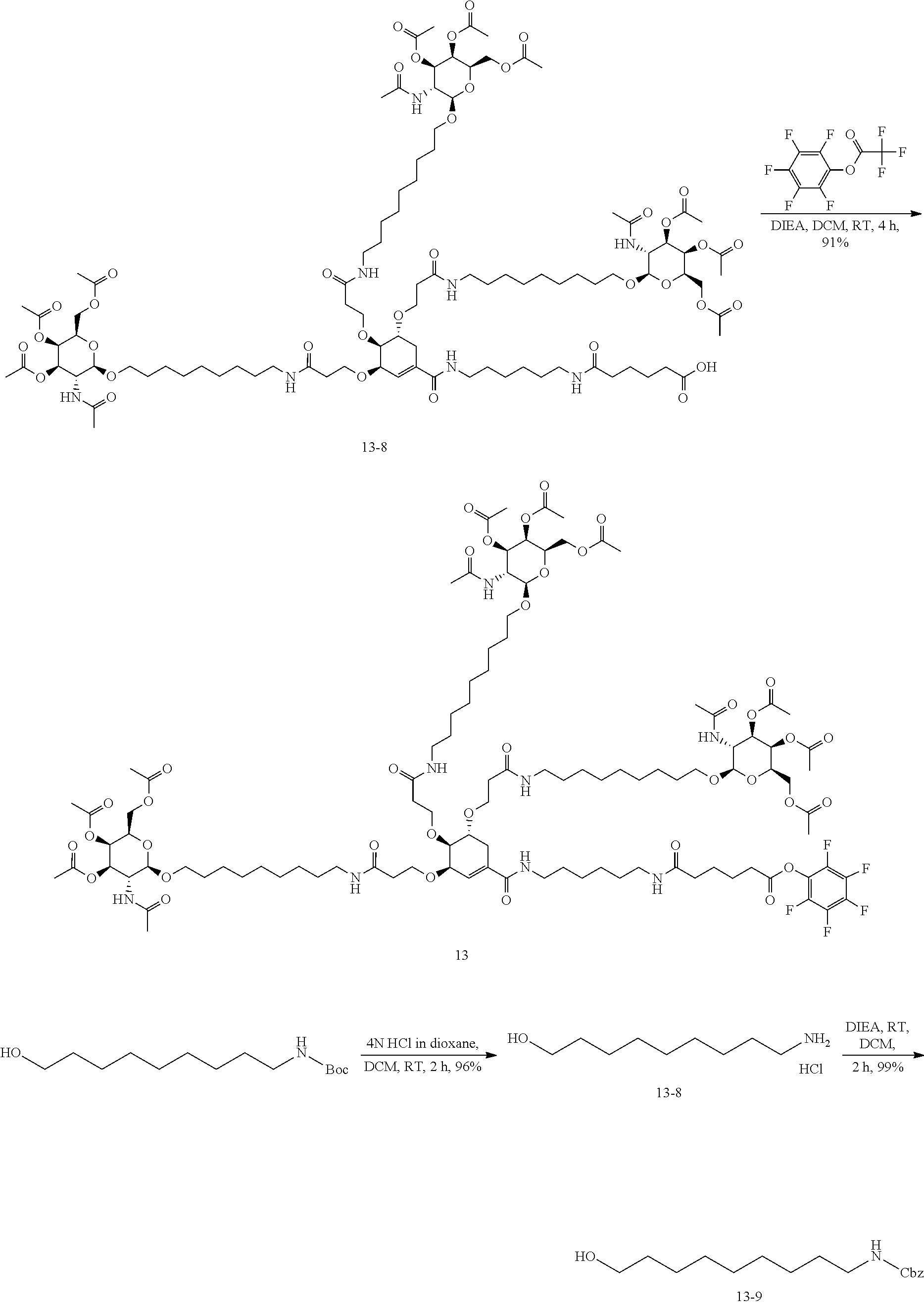

TMS-OTf (25.5 mL, 141.26 mmol) was added to (2S,3R,4R,5R,6R)-3-acetamido-6-(acetoxymethyl)tetrahydro-2H-pyran-2,4,5-triyl triacetate (50 g, 128.42 mmol) in DCE (500 mL) at RT. The resulting mixture was stirred at 60° C. for 1 hour. After cooling to RT, TEA (26.8 mL, 192.63 mmol) was added dropwise to the mixture and stirred for 10 minutes. The reaction mixture was diluted with DCM (250 mL) and washed sequentially with saturated NaHCO₃ (500 mL) and saturated brine (500 mL). The organic layer was dried over Na₂SO₄, filtered and evaporated to afford (5R,6R,7R,7aR)-5-(acetoxymethyl)-2-methyl-3a,6,7,7a-tetrahydro-5H-pyrano[3,2-d]oxazole-6,7-diyl diacetate (13-2) (50.0 g, 118%) as a brown oil. The product was used in the next step without further purification.

1H-NMR (500 MHz, DMSO, 25° C.) δ 1.95 (d, J=1.4 Hz, 3H), 2.01 (d, J=3.4 Hz, 6H), 2.07 (s, 3H), 3.95 (m, 1H), 4.02-4.17 (m, 2H), 4.26 (m, 1H), 4.89 (m, 1H), 5.24 (m, 1H), 6.05 (d, J=7.0 Hz, 1H). LCMS m/z Expected 329.1, Observed 330.1.

HCl (4 N in dioxane) (90 mL, 360.00 mmol) was added to tert-butyl (9-hydroxynonyl)carbamate (9 g, 34.70 mmol) in DCM (90 mL). The resulting solution was stirred at RT for 2 hours. The solvent was removed under reduced pressure to afford 9-aminononan-1-ol hydrochloride (13-8) (7.70 g, 96%) as a white solid. The product was used in the next step without further purification.

1H-NMR (300 MHz, CDCl₃, 22° C.) δ 1.26 (s, 10H), 1.35-1.45 (m, 2H), 1.55 (m, 2H), 2.67-2.79 (m, 2H), 3.37 (t, J=6.5 Hz, 2H), 4.07-4.54 (m, 1H). LCMS m/z Expected 159.2, Observed 160.1.

DIEA (24.82 mL, 142.13 mmol) was added to 9-aminononan-1-ol hydrochloride (11 g, 47.38 mmol) in DCM (200 mL) over a period of 10 minutes. Benzyl (2,5-dioxopyrrolidin-1-yl) carbonate (14.76 g, 59.22 mmol) was added to the mixture. The resulting solution was stirred at RT for 2 hours. The solvent was removed under reduced pressure. The crude product was purified by flash silica chromatography, elution gradient 0 to 5% MeOH in DCM. Pure fractions were evaporated to dryness to afford benzyl (9-hydroxynonyl)carbamate (13-9) (13.75 g, 99%) as a white solid.

1H-NMR (300 MHz, CDCl3, 22° C.) δ 1.23 (s, 10H), 1.38 (m, 4H), 2.96 (q, J=6.6 Hz, 2H), 3.33-3.4 (m, 2H), 4.32 m, J=5.2, 1.7 Hz, 1H), 4.99 (s, 2H), 7.18-7.25 (m, 1H), 7.28-7.36 (m, 5H). LCMS m/z Expected 293.2, Observed 294.1.

Benzyl (9-hydroxynonyl)carbamate (13-9, 14.34 g, 48.89 mmol) was added to (5R,6R,7R,7aR)-5-(acetoxymethyl)-2-methyl-3a,6,7,7a-tetrahydro-5H-pyrano[3,2-d]oxazole-6,7-diyl diacetate (13-2, 23 g, 48.89 mmol) and molecular sieves (2.3 g) in DCE (250 mL) at RT. TMS-OTf (8.83 mL, 48.89 mmol) was added after the reaction had been stirred for 30 minutes at RT. The resulting mixture was stirred at 60° C. for 2 hours. The reaction mixture was quenched with saturated NaHCO₃ (300 mL), extracted with DCM (3×250 mL), and washed with saturated brine (2×300 mL). The organic layer was dried over Na₂SO₄, filtered and evaporated to afford dark brown oil. The crude product was purified by flash silica chromatography, elution gradient 0 to 80% EtOAc in petroleum ether. Pure fractions were evaporated to dryness to afford (2R,3R,4R,5R,6R)-5-acetamido-2-(acetoxymethyl)-6-((9-(((benzyloxy)carbonyl)amino)nonyl)oxy)tetrahydro-2H-pyran-3,4-diyl diacetate (13-3) (27.0 g, 89%) as a yellow liquid.

1H-NMR (300 MHz, CDCl₃, 22° C.) δ 1.24 (s, 10H), 1.34-1.5 (m, 4H), 1.77 (s, 3H), 1.90 (s, 3H), 2.00 (d, J=1.5 Hz, 3H), 2.11 (s, 3H), 2.97 (q, J=6.6 Hz, 2H), 3.36-3.46 (m, 1H), 3.70 (m, J=9.8, 6.1 Hz, 1H), 3.8-4.02 (m, 2H), 4.06-4.16 (m, 1H), 4.48 (d, J=8.5 Hz, 1H), 4.98 (d, J=14.4 Hz, 3H), 5.23 (m, J=9.9, 3.6 Hz, 2H), 7.19-7.29 (m, 1H), 7.31-7.37 (m, 5H), 7.83 (d, J=9.2 Hz, 1H). LCMS m/z Expected 622.3, Observed 623.3.

Iodotrimethylsilane (17.14 mL, 120.44 mmol) was added to (2R,3R,4R,5R,6R)-5-acetamido-2-(acetoxymethyl)-6-((9-(((benzyloxy)carbonyl)amino)nonyl)oxy)tetrahydro-2H-pyran-3,4-diyl diacetate (13-3, 15 g, 24.09 mmol) in MeCN (150 mL) at RT. The resulting mixture was stirred at RT for 15 minutes. The solvent was removed under reduced pressure. The crude product was purified by flash C18-flash chromatography, elution gradient 0 to 35% MeCN in water (0.1% TFA). Pure fractions were evaporated to dryness to afford (2R,3R,4R,5R,6R)-5-acetamido-2-(acetoxymethyl)-6-((9-aminononyl)oxy)tetrahydro-2H-pyran-3,4-diyl diacetate (13-4) (14.00 g, 119%) as a yellow gum.

1H-NMR (300 MHz, CDCl₃, 22° C.) δ 1.26 (d, J=6.6 Hz, 10H), 1.50 (m, J=16.1, 8.2 Hz, 4H), 1.77 (s, 3H), 1.89 (s, 3H), 2.00 (s, 3H), 2.10 (s, 3H), 2.69-2.84 (m, 2H), 3.41 (m, J=9.8, 6.4 Hz, 1H), 3.70 (m, J=9.7, 6.1 Hz, 1H), 3.87 (m, J=11.2, 8.8 Hz, 1H), 3.97-4.06 (m, 3H), 4.48 (d, J=8.4 Hz, 1H), 4.96 (m, J=11.2, 3.4 Hz, 1H), 5.22 (d, J=3.4 Hz, 1H), 7.84 (d, J=9.3 Hz, 1H). LCMS m/z Expected 488.3, Observed 489.3.

DIEA (3.65 mL, 20.88 mmol) was added to (2R,3R,4R,5R,6R)-5-acetamido-2-(acetoxymethyl)-6-((9-aminononyl)oxy)tetrahydro-2H-pyran-3,4-diyl diacetate (13-4, 6.12 g, 12.53 mmol), 3,3′,3″-(((1R,2S,3R)-5-((6-(((benzyloxy)carbonyl)amino)hexyl)carbamoyl) cyclohex-4-ene-1,2,3-triyl)tris(oxy))tripropionic acid (13-5, 1.3 g, 2.09 mmol), BOP (3.69 g, 8.35 mmol) and DMAP (0.765 g, 6.26 mmol) in DMF (70 mL) under nitrogen. The resulting mixture was stirred at RT for 16 hours. The solvent was removed under reduced pressure. The crude product was purified by flash C18-flash chromatography, elution gradient 0 to 63% MeCN in water (0.1% TFA). Pure fractions were evaporated to dryness to afford [(2R,3R,4R,5R,6R)-5-acetamido-3,4-diacetoxy-6-[9-[3-[(1R,5R,6S)-5,6-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-3-[6-(benzyloxycarbonylamino)hexylcarbamoyl]cyclohex-3-en-1-yl]oxypropanoylamino]nonoxy]tetrahydropyran-2-yl]methyl acetate (13-6) (2.400 g, 56%) as a yellow solid.

1H-NMR (300 MHz, CDCl3, 22° C.) δ 1.23 (s, 34H), 1.41 (d, J=24.1 Hz, 16H), 1.76 (s, 9H), 1.89 (s, 9H), 1.99 (s, 9H), 2.10 (s, 9H), 2.12-2.17 (m, 1H), 2.2-2.35 (m, 7H), 2.99 (d, J=14.4 Hz, 10H), 3.40 (m, J=9.4, 6.3 Hz, 4H), 3.63-3.76 (m, 11H), 4.08 (d, J=19.3 Hz, 12H), 4.48 (d, J=8.5 Hz, 3H), 4.92-5.02 (m, 5H), 5.21 (d, J=3.4 Hz, 3H), 6.27 (s, 1H), 7.23 (t, J=5.6 Hz, 1H), 7.28-7.37 (m, 5H), 7.76 (t, J=5.4 Hz, 1H), 7.82 (m, J=7.9, 4.9 Hz, 6H). LCMS m/z Expected 2033.1, Observed 1018.1 (M+1, z=2).

TMS-I (1.004 mL, 7.37 mmol) was added to [(2R,3R,4R,5R,6R)-5-acetamido-3,4-diacetoxy-6-[9-[3-[(1R,5R,6S)-5,6-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-3-[6-(benzyloxycarbonylamino)hexylcarbamoyl]cyclohex-3-en-1-yl]oxypropanoylamino]nonoxy]tetrahydropyran-2-yl]methyl acetate (13-6, 1.5 g, 0.74 mmol) in MeCN (20 mL) at RT. The reaction mixture was stirred for 15 minutes at RT. The solvent was removed under reduced pressure. DCM (50 ml×4) was added to the residue and concentrated to dryness to afford [(2R,3R,4R,5R,6R)-5-acetamido-3,4-diacetoxy-6-[9-[3-[(1R,5R,6S)-5,6-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-3-(6-aminohexylcarbamoyl)cyclohex-3-en-1-yl]oxypropanoylamino]nonoxy]tetrahydropyran-2-yl]methyl acetate (13-7) (1.3 g, 0.684 mmol, 93%) as a brown solid. The product was used in the next step without further purification.

1H-NMR (500 MHz, DMSO, 25° C.) δ 1.21-1.28 (m, 30H), 1.29-1.41 (m, 15H), 1.48-1.57 (m, 7H), 1.76 (s, 3H), 1.80 (s, 6H), 1.89 (d, J=2.0 Hz, 9H), 1.99 (d, J=1.9 Hz, 10H), 2.10 (s, 10H), 2.25 (q, J=6.8 Hz, 4H), 2.32 (t, J=6.6 Hz, 2H), 2.71-2.81 (m, 2H), 2.98-3.03 (m, 6H), 3.07 (t, J=6.8 Hz, 2H), 3.36-3.42 (m, 3H), 3.54-3.62 (m, 3H), 3.66 (d, J=3.5 Hz, 1H), 3.68-3.76 (m, 6H), 3.99-4.04 (m, 6H), 4.13 (t, J=6.5 Hz, 2H), 4.16-4.22 (m, 2H), 4.48 (d, J=8.1 Hz, 1H), 4.64 (d, J=16.3 Hz, 1H), 4.71-4.85 (m, 3H), 4.94-5.03 (m, 3H), 5.2-5.32 (m, 3H), 6.27 (s, 1H), 7.28-7.33 (m, 1H), 7.4-7.57 (m, 3H), 7.76-7.83 (m, 3H), 7.98 (d, J=8.1 Hz, 2H). LCMS m/z Expected 1899.0, Observed 951.1 (M+1, z=2).

DIEA (0.956 mL, 5.47 mmol) was added to [(2R,3R,4R,5R,6R)-5-acetamido-3,4-diacetoxy-6-[9-[3-[(1R,5R,6S)-5,6-bis[3-[9-[(2R,3R,4R,5R,6R)-3-acetamido-4,5-diacetoxy-6-(acetoxymethyl)tetrahydropyran-2-yl]oxynonylamino]-3-oxo-propoxy]-3-(6-aminohexylcarbamoyl)cyclohex-3-en-1-yl]oxypropanoylamino]nonoxy]tetrahydropyran-2-yl]methyl acetate (13-7, 1.3 g, 0.68 mmol), O-(Benzotriazol-1-yl)-N—N—N′,N′-tetramethyluronium hexafluorophosphate (1.557 g, 4.10 mmol) and adipic acid (1.000 g, 6.84 mmol) in DMF (20 mL) at RT under nitrogen. The resulting mixture was stirred at RT for 16 hours. The solvent was removed under reduced pressure to afford the crude product. The crude product was purified by flash C18-flash chromatography, elution gradient 0 to 50% MeCN in water (0.1% TFA). The product was not clean enough, it was purified by flash C18-flash chromatography, elution gradient 0 to 43% MeCN in water (0.1% TFA). Pure fractions were evaporated to dryness to afford 6-oxo-6-((6-((3R,4S,5R)-3,4,5-tris(3-((9-(((2R,3R,4R,5R,6R)-3-acetamido-4,5-diacetoxy-6-(acetoxymethyl)tetrahydro-2H-pyran-2-yl)oxy)nonyl)amino)-3-oxopropoxy)cyclohex-1-ene-1-carboxamido)hexyl)amino)hexanoic acid (13-8) (0.303 g, 22%) as a white solid.

1H-NMR (500 MHz, DMSO, 24° C.) δ 1.24 (t, J=5.6 Hz, 29H), 1.34 (d, J=21.3 Hz, 15H), 1.43-1.49 (m, 5H), 1.53 (p, J=6.6 Hz, 5H), 1.78 (d, J=19.9 Hz, 9H), 1.89 (d, J=1.7 Hz, 9H), 1.99 (s, 9H), 2.04 (t, J=6.8 Hz, 2H), 2.10 (s, 10H), 2.19 (t, J=6.9 Hz, 2H), 2.22-2.29 (m, 4H), 2.31 (t, J=6.6 Hz, 2H), 2.34-2.41 (m, 1H), 2.97-3.07 (m, 10H), 3.35-3.43 (m, 3H), 3.56-3.6 (m, 2H), 3.63-3.76 (m, 8H), 3.98-4.05 (m, 7H), 4.13 (t, J=6.4 Hz, 3H), 4.15-4.24 (m, 3H), 4.48 (d, J=8.5 Hz, 1H), 4.83 (d, J=3.5 Hz, 3H), 4.93-5.03 (m, 3H), 5.21-5.32 (m, 3H), 6.27 (s, 1H), 7.71-7.84 (m, 5H), 7.98 (d, J=8.1 Hz, 3H). LCMS m/z Expected 2027.1, Observed 1014.8 (M+1, z=2).

Perfluorophenyl 2,2,2-trifluoroacetate (0.065 mL, 0.38 mmol) was added to (13-8) (110 mg, 0.05 mmol) and DIEA (0.095 mL, 0.54 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (13) (108 mg, 91%) as off white solid. The product was used in the next reaction without any further purification. LCMS m/z Expected 2193.05, Observed 1098.1 [M+H]+(z=2).

### Synthesis of Precursor (14)

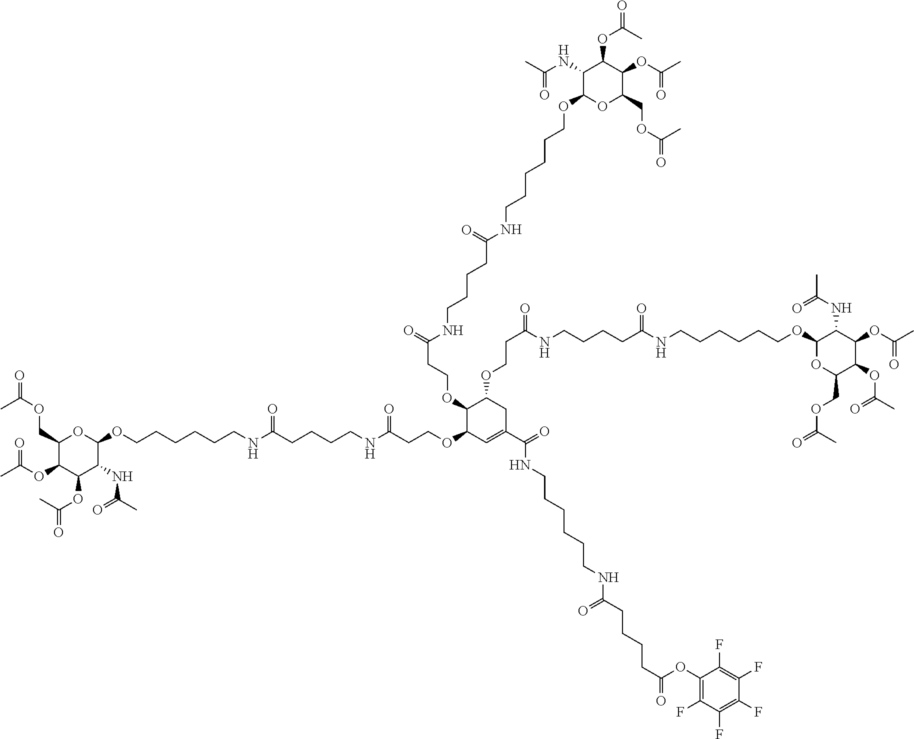

### Synthetic Scheme:

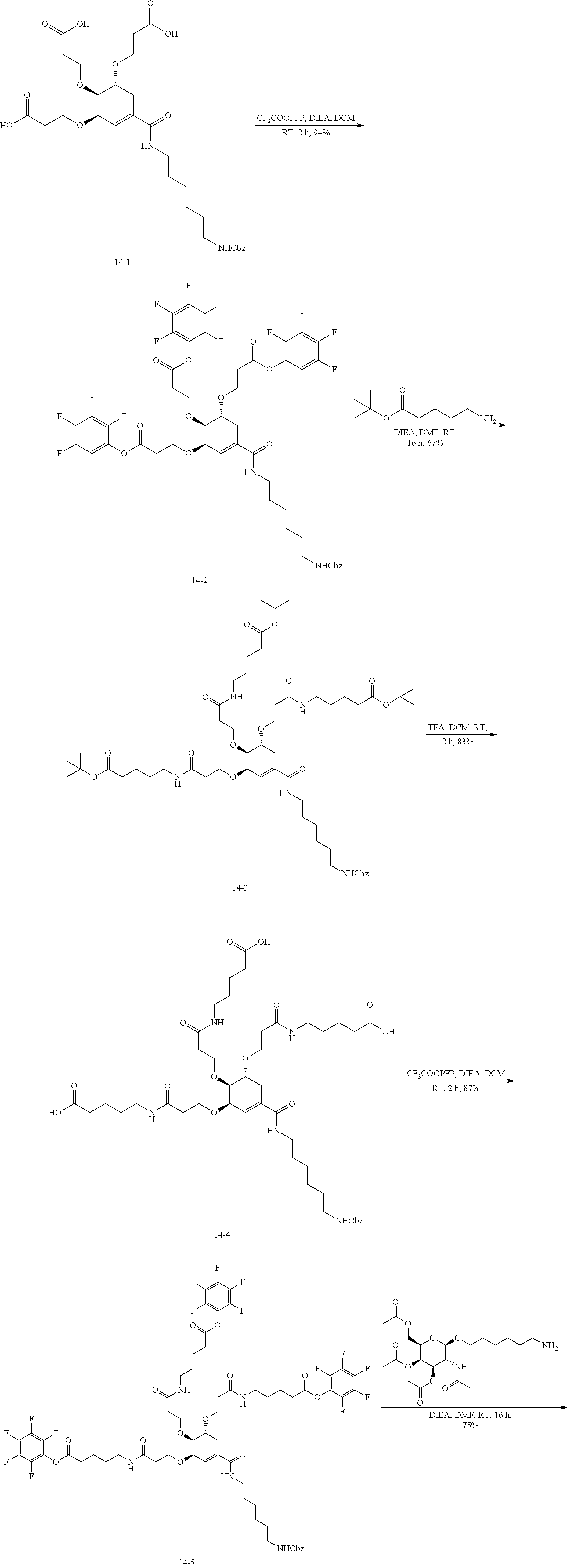

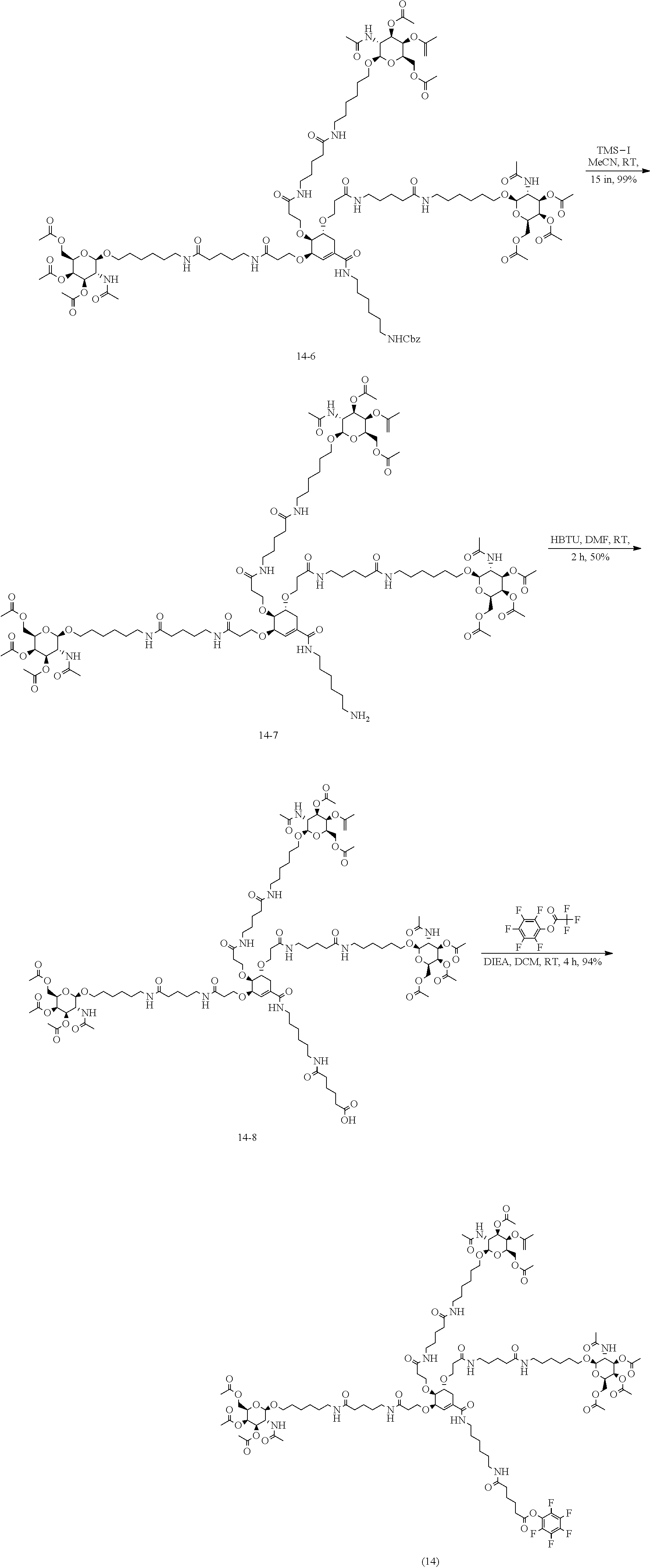

Perfluorophenyl 2,2,2-trifluoroacetate (1.738 mL, 10.12 mmol) was added to 3,3,3″-(((1R,2S,3R)-5-((6-(((benzyloxy)carbonyl)amino)hexyl)carbamoyl)cyclohex-4-ene-1,2,3-triyl)tris(oxy))tripropionic acid (14-1) (1.4 g, 2.25 mmol) and DIEA (2.55 mL, 14.61 mmol) in DCM (30 mL) at 0° C. The resulting mixture was stirred at RT for 2 hours. The reaction mixture was diluted with DCM (150 ml) and washed sequentially with 1 M NaHSO₄ (2×100 ml), saturated NaHCO₃ (2×100 ml), and saturated brine (100 ml). The organic layer was dried over Na2SO4, filtered and evaporated to afford tris(perfluorophenyl) 3,3′,3″-(((1R,2S,3R)-5-((6-(((benzyloxy)carbonyl)amino)hexyl)carbamoyl)cyclohex-4-ene-1,2,3-triyl)tris(oxy))tripropionate (14-2) (2.400 g, 95%) as a yellow oil. The product was used in the next step directly without further purification.

1H-NMR (500 MHz, DMSO, 25° C.) δ 1.25 (d, J=6.6 Hz, 8H), 2.15-2.25 (m, 1H), 2.37-2.48 (m, 2H), 2.96-3.06 (m, 8H), 3.71-3.99 (m, 9H), 4.12-4.2 (m, 1H), 4.99 (s, 2H), 6.3-6.37 (m, 1H), 7.21 (t, J=5.7 Hz, 1H), 7.3-7.36 (m, 5H), 7.83-7.91 (m, 1H). LCMS m/z, Expected 1120.2, Observed 1143.3.

DIEA (2.69 mL, 15.39 mmol) was added to tris(perfluorophenyl) 3,3′,3″-(((1R,2S,3R)-5-((6-(((benzyloxy)carbonyl)amino)hexyl)carbamoyl)cyclohex-4-ene-1,2,3-triyl)tris(oxy))tripropionate (2.3 g, 2.05 mmol) and tert-butyl 5-aminopentanoate (1.600 g, 9.23 mmol) at RT in DMF (30 mL). The resulting mixture was stirred at RT for 16 hours. The solvent was removed under reduced pressure to afford crude product. The crude product was purified by flash C18-flash chromatography, elution gradient 0 to 90% MeCN in water (0.1% NH₄HCO₃). Pure fractions were evaporated to dryness to afford tri-tert-butyl 5,5′,5″-((3,3′,3″-(((1R,2S,3R)-5-((6-(((benzyloxy)carbonyl)amino)hexyl)carbamoyl)cyclohex-4-ene-1,2,3-triyl)tris(oxy))tris(propanoyl))tris(azanediyl))tripentanoate (14-3) (1.500 g, 67%) as a yellow oil.

1H-NMR (300 MHz, DMSO, 23° C.) δ 1.3-1.47 (m, 48H), 2.15-2.28 (m, 10H), 2.87-3.09 (m, 12H), 3.56-3.8 (m, 9H), 4.00 (s, 1H), 4.99 (s, 2H), 6.26 (s, 1H), 7.27-7.35 (m, 5H), 7.69-7.9 (m, 5H). LCMS m/z Expected 1087.7, Observed 1110.7.

TFA (10 mL) was added to tri-tert-butyl 5,5′,5″-((3,3′,3″-(((1R,2S,3R)-5-((6-(((benzyloxy)carbonyl)amino)hexyl)carbamoyl)cyclohex-4-ene-1,2,3-triyl)tris(oxy))tris(propanoyl))tris(azanediyl))tripentanoate (14-3) (1.4 g, 1.29 mmol) in DCM (30 mL) at RT. The resulting mixture was stirred at RT for 2 hours. The solvent was removed under reduced pressure to afford 5,5′,5″-((3,3′,3″-(((1R,2S,3R)-5-((6-(((benzyloxy)carbonyl) amino)hexyl)carbamoyl)cyclohex-4-ene-1,2,3-triyl)tris(oxy))tris(propanoyl))tris(azanediyl)) tripentanoic acid (14-4) (1.100 g, 83%) as a yellow oil. The product was used in the next step directly without further purification.

1H-NMR (500 MHz, DMSO, 25° C.) δ 1.2-1.27 (m, 5H), 1.35-1.42 (m, 10H), 1.44-1.51 (m, 6H), 2.17-2.22 (m, 6H), 2.23-2.29 (m, 4H), 2.32 (t, J=6.5 Hz, 2H), 2.94-3.09 (m, 11H), 3.63-3.78 (m, 8H), 3.99-4.04 (m, 1H), 5.00 (s, 2H), 6.27 (d, J=2.9 Hz, 1H), 7.21 (t, J=5.7 Hz, 1H), 7.27-7.38 (m, 5H), 7.74-7.87 (m, 4H), 10.59 (brs, 3H). LCMS m/z Expected 919.5, Observed 920.2.

Perfluorophenyl 2,2,2-trifluoroacetate (0.748 mL, 4.35 mmol) was added to 5,5′,5″-((3,3′,3″-(((1R,2S,3R)-5-((6-(((benzyloxy)carbonyl)amino)hexyl)carbamoyl)cyclohex-4-ene-1,2,3-triyl)tris(oxy))tris(propanoyl))tris(azanediyl))tripentanoic acid (14-4) (1 g, 0.97 mmol) and DIEA (1.098 mL, 6.29 mmol) in DCM (30 mL) at 0° C. The resulting mixture was stirred at RT for 2 hours. The reaction mixture was diluted with DCM (80 ml) and washed sequentially with 1 M NaHSO₄ (2×70 ml), saturated NaHCO₃ (2×70 ml), and saturated brine (100 ml).

The organic layer was dried over Na₂SO₄, filtered and evaporated to afford tris(perfluorophenyl) 5,5′,5″-((3,3′,3″-(((1R,2S,3R)-5-((6-(((benzyloxy)carbonyl)amino)hexyl) carbamoyl)cyclohex-4-ene-1,2,3-triyl)tris(oxy))tris(propanoyl))tris(azanediyl))tripentanoate (14-5) (1.200 g, 87%) as a yellow oil. The product was used in the next step directly without further purification.

1H-NMR (500 MHz, DMSO, 25° C.) δ 1.17-1.28 (m, 6H), 1.38 (q, J=7.3, 6.8 Hz, 5H), 1.43-1.52 (m, 6H), 1.6-1.69 (m, 5H), 2.1-2.41 (m, 9H), 2.71-2.82 (m, 5H), 2.96 (q, J=6.6 Hz, 2H), 3.02-3.11 (m, 7H), 3.62-3.81 (m, 8H), 4.99 (d, J=1.8 Hz, 2H), 6.28 (s, 1H), 7.21 (t, J=5.7 Hz, 1H), 7.26-7.39 (m, 5H), 7.75-7.92 (m, 4H). LCMS m/z Expected 1417.4, Observed 1418.4.

DIEA (1.108 mL, 6.35 mmol) was added to tris(perfluorophenyl) 5,5′,5″-((3,3′,3″-(((1R,2S,3R)-5-((6-(((benzyloxy)carbonyl)amino)hexyl)carbamoyl)cyclohex-4-ene-1,2,3-triyl)tris(oxy))tris(propanoyl))tris(azanediyl))tripentanoate (14-5) (1.2 g, 0.85 mmol) and (2R,3R,4R,5R,6R)-5-acetamido-2-(acetoxymethyl)-6-((6-aminohexyl)oxy)tetrahydro-2H-pyran-3,4-diyl diacetate (2.187 g, 3.81 mmol) in DMF (20 mL) at RT. The resulting mixture was stirred at RT for 16 hours. The solvent was removed under reduced pressure to afford crude product. The crude product was purified by flash C18-flash chromatography, elution gradient 0 to 60% MeCN in water (0.1% TFA). Pure fractions were evaporated to dryness to afford [(2R,3R,4R,5R,6R)-5-acetamido-3,4-diacetoxy-6-[6-[5-[3-[(1R,5R,6S)-5,6-bis[3-[[5-[6-[(2R,3R,4R,5R,6R)-3-acetamido-4,5-diacetoxy-6-(acetoxymethyl)tetrahydropyran-2-yl]oxyhexylamino]-5-oxo-pentyl]amino]-3-oxo-propoxy]-3-[6-(benzyloxycarbonylamino) hexylcarbamoyl]cyclohex-3-en-1-yl]oxypropanoylamino]pentanoylamino]hexoxy]tetrahydropyran-2-yl]methyl acetate (14-6) (1.400 g, 75%) as a grey solid.

1H-NMR (300 MHz, DMSO, 22° C.) δ 1.06-1.28 (m, 18H), 1.29-1.47 (m, 27H), 1.76 (s, 9H), 1.88 (s, 9H), 1.98 (s, 9H), 2.09 (s, 9H), 2.18-2.36 (m, 8H), 2.99 (d, J=7.8 Hz, 17H), 3.32-3.46 (m, 4H), 3.59-3.75 (m, 11H), 3.8-3.91 (m, 4H), 4.01 (s, 11H), 4.47 (d, J=8.5 Hz, 3H), 4.93 (d, J=3.4 Hz, 2H), 4.95-5.01 (m, 4H), 5.20 (d, J=3.4 Hz, 3H), 6.26 (s, 1H), 7.21 (d, J=5.6 Hz, 1H), 7.28-7.36 (m, 5H), 7.73 (t, J=5.6 Hz, 3H), 7.83 (t, J=8.6 Hz, 7H). LCMS m/z Expected 2204.1, Observed 1103.3.

TMS-I (0.432 mL, 3.17 mmol) was added to [(2R,3R,4R,5R,6R)-5-acetamido-3,4-diacetoxy-6-[6-[5-[3-[(1R,5R,6S)-5,6-bis[3-[[5-[6-[(2R,3R,4R,5R,6R)-3-acetamido-4,5-diacetoxy-6-(acetoxymethyl)tetrahydropyran-2-yl]oxyhexylamino]-5-oxo-pentyl]amino]-3-oxo-propoxy]-3-[6-(benzyloxycarbonylamino)hexylcarbamoyl]cyclohex-3-en-1-yl]oxypropanoylamino]pentanoylamino]hexoxy]tetrahydropyran-2-yl]methyl acetate (14-6) (700 mg, 0.32 mmol) in MeCN (20 mL) at RT. The resulting mixture was stirred at RT for 15 minutes. The solvent was removed under reduced pressure. DCM (50 ml×4) was added to the residue and concentrated to dryness to afford [(2R,3R,4R,5R,6R)-5-acetamido-3,4-diacetoxy-6-[6-[5-[3-[(1R,5R,6S)-5,6-bis[3-[[5-[6-[(2R,3R,4R,5R,6R)-3-acetamido-4,5-diacetoxy-6-(acetoxymethyl)tetrahydropyran-2-yl]oxyhexylamino]-5-oxo-pentyl]amino]-3-oxo-propoxy]-3-(6-aminohexylcarbamoyl)cyclohex-3-en-1-yl]oxypropanoylamino]pentanoylamino]hexoxy]tetrahydropyran-2-yl]methyl acetate (14-7) (650 mg, 99%) as a yellow solid. The product was used in the next step directly without further purification.

1H-NMR (500 MHz, DMSO, 25° C.) δ 1.11-1.56 (m, 51H), 1.78 (d, J=19.1 Hz, 9H), 1.88 (d, J=1.9 Hz, 9H), 1.98 (s, 9H), 2.01-2.05 (m, 8H), 2.09 (s, 9H), 2.25 (q, J=6.5 Hz, 4H), 2.76 (q, J=6.8 Hz, 2H), 2.91-3.11 (m, 15H), 3.3-3.46 (m, 3H), 3.65-3.74 (m, 9H), 3.82-3.89 (m, 2H), 3.98-4.04 (m, 10H), 4.43-4.49 (m, 2H), 4.95 (dd, J=11.2, 3.5 Hz, 2H), 5.20 (d, J=3.5 Hz, 2H), 6.26 (s, 1H), 7.72-7.87 (m, 9H), 7.98 (d, J=8.0 Hz, 1H). Two protons have been exchanged. LCMS m/z Expected 2070.1, Observed 1036.3 (M+1, z=2).

DIEA (0.548 mL, 3.14 mmol) was added to [(2R,3R,4R,5R,6R)-5-acetamido-3,4-diacetoxy-6-[6-[5-[3-[(1R,5R,6S)-5,6-bis[3-[[5-[6-[(2R,3R,4R,5R,6R)-3-acetamido-4,5-diacetoxy-6-(acetoxymethyl)tetrahydropyran-2-yl]oxyhexylamino]-5-oxo-pentyl]amino]-3-oxo-propoxy]-3-(6-aminohexylcarbamoyl)cyclohex-3-en-1-yl]oxypropanoylamino]pentanoylamino]hexoxy]tetrahydropyran-2-yl]methyl acetate (14-7) (650 mg, 0.31 mmol), adipic acid (229 mg, 1.57 mmol) and O-(Benzotriazol-1-yl)-N—N—N′,N′-tetramethyluronium hexafluorophosphate (357 mg, 0.94 mmol) in DMF (15 mL) at RT under nitrogen. The resulting mixture was stirred at RT for 2 hours. The solvent was removed under reduced pressure to afford crude product. The crude product was purified by flash C18-flash chromatography, elution gradient 0 to 50% MeCN in water (0.1% TFA). Pure fractions were evaporated to dryness to afford 6-oxo-6-((6-((3R,4S,5R)-3,4,5-tris(3-((5-((6-(((2R,3R,4R,5R,6R)-3-acetamido-4,5-diacetoxy-6-(acetoxymethyl)tetrahydro-2H-pyran-2-yl)oxy)hexyl)amino)-5-oxopentyl)amino)-3-oxopropoxy)cyclohex-1-ene-1-carboxamido)hexyl)amino)hexanoic acid (14-8) (344 mg, 50%) as a yellow solid.

1H-NMR (500 MHz, DMSO, 25° C.) δ 1.13-1.28 (m, 15H), 1.3-1.55 (m, 32H), 1.79 (d, J=19.4 Hz, 9H), 1.89 (s, 9H), 1.99 (s, 9H), 2.01-2.05 (m, 8H), 2.10 (s, 9H), 2.12-2.14 (m, 1H), 2.19 (t, J=6.9 Hz, 2H), 2.22-2.28 (m, 4H), 2.3-2.41 (m, 3H), 2.91-3.09 (m, 17H), 3.3-3.44 (m, 3H), 3.54-3.64 (m, 1H), 3.66-3.8 (m, 9H), 3.84-3.89 (m, 2H), 3.92-4.07 (m, 10H), 4.08-4.24 (m, 2H), 4.48 (d, J=8.4 Hz, 2H), 4.83 (d, J=3.5 Hz, 1H), 4.92-5.03 (m, 3H), 5.21 (d, J=3.4 Hz, 2H), 5.32 (d, J=3.2 Hz, 1H), 6.27 (s, 1H), 7.58-7.91 (m, 10H), 7.99 (d, J=8.1 Hz, 1H). One proton have been exchanged. LCMS m/z Expected 2198.1, Observed 1100.6 (M+1, z=2).

Perfluorophenyl 2,2,2-trifluoroacetate (0.109 mL, 0.64 mmol) was added to (14-8) (200 mg, 0.09 mmol) and DIEA (0.159 mL, 0.91 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (14) (202.0 mg, 89%) as white solid. The product was used in the next reaction without any further purification. LCMS m/z Expected 2364.1, Observed 1183.6 [M+H]+(z=2).

### Synthesis of Precursor (15)

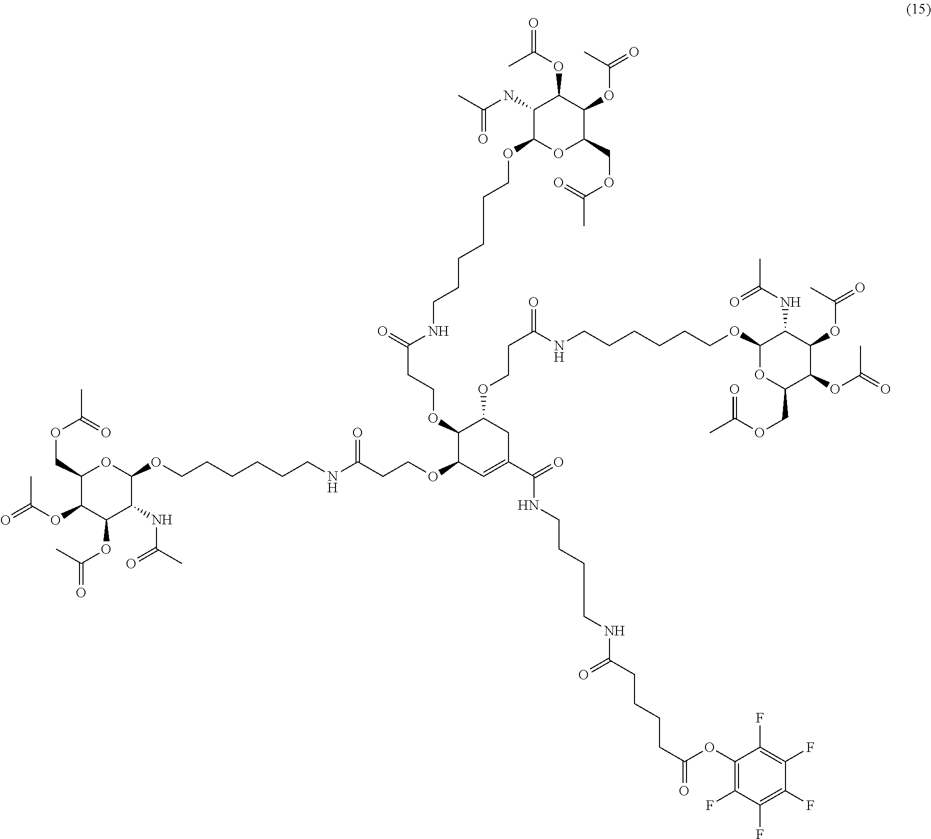 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)NCCCCNC(=O)CCCCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Synthetic Scheme:

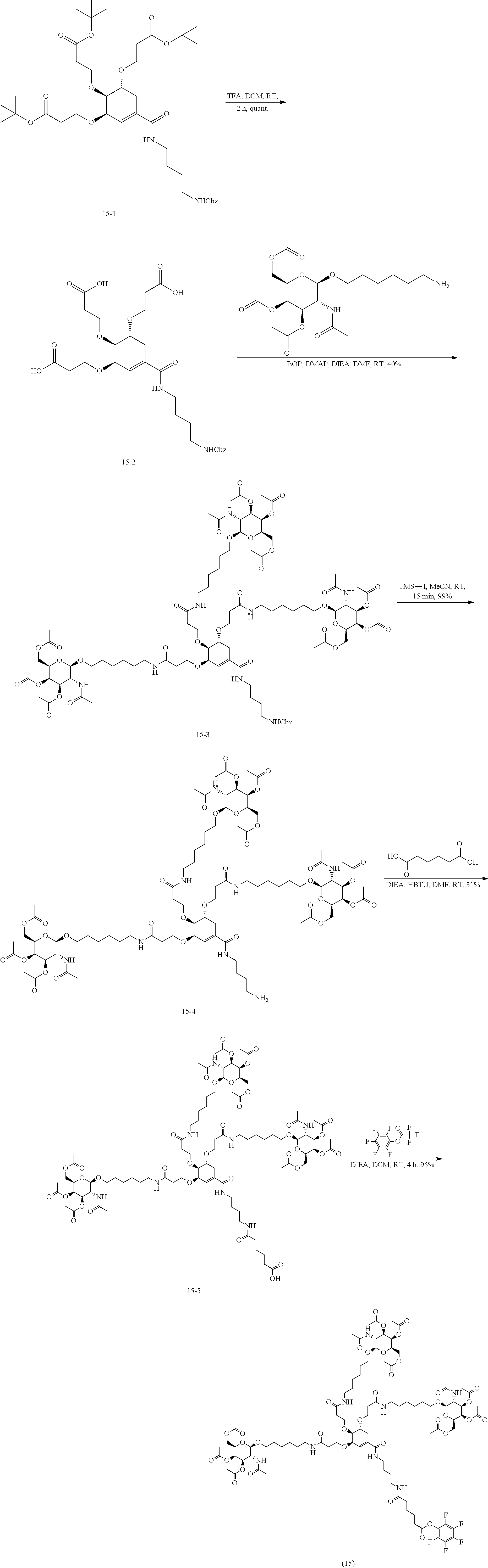

TFA (20 mL) was added to tri-tert-butyl 3,3′,3″-(((1R,2S,3R)-5-((4-(((benzyloxy)carbonyl)amino)butyl)carbamoyl)cyclohex-4-ene-1,2,3-triyl)tris(oxy))tripropionate (15-1) (2 g, 2.62 mmol) in DCM (20 mL) at RT. The resulting mixture was stirred at RT for 2 hours. The solvent was removed under reduced pressure to afford 3,3′,3″-(((1R,2S,3R)-5-((4-(((benzyloxy)carbonyl)amino)butyl)carbamoyl)cyclohex-4-ene-1,2,3-triyl)tris(oxy))tripropionic acid (15-2) (2 g, 3.36 mmol, 99%) as a yellow oil. The product was used in the next step directly without further purification.

1H-NMR (300 MHz, DMSO, 23° C.) δ 1.37-1.44 (m, 4H), 2.08-2.33 (m, 2H), 2.43 (m, J=6.7, 5.2, 2.6 Hz, 6H), 3.03 (m, J=24.2, 5.7 Hz, 4H), 3.71-3.79 (m, 8H), 4.08 (s, 1H), 5.01 (s, 2H), 6.21-6.32 (m, 1H), 7.23 (d, J=5.8 Hz, 1H), 7.32-7.36 (m, 5H), 7.91 (t, J=5.6 Hz, 1H), 12.10 (s, 3H). LCMS m/z Expected 594.2, Observed 595.3.

DIEA (5.87 mL, 33.64 mmol) was added to 3,3′,3″-(((1R,2S,3R)-5-((4-(((benzyloxy)carbonyl)amino)butyl)carbamoyl)cyclohex-4-ene-1,2,3-triyl)tris(oxy))tripropionic acid (15-2) (2 g, 3.36 mmol), (2R,3R,4R,5R,6R)-5-acetamido-2-(acetoxymethyl)-6-((6-aminohexyl)oxy)tetrahydro-2H-pyran-3,4-diyl diacetate (6.61 g, 14.80 mmol), BOP (5.95 g, 13.45 mmol) and DMAP (1.233 g, 10.09 mmol) in DMF (30 mL) at RT under nitrogen. The resulting mixture was stirred at RT for 16 hours. The solvent was removed under reduced pressure to afford the crude product. The crude product was purified by flash C18-flash chromatography, elution gradient 0 to 60% MeCN in water (0.1% TFA). Pure fractions were evaporated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[4-(benzyloxycarbonylamino)butylcarbamoyl]cyclohex-3-en-1-yl]oxypropanoylamino]hexoxy]tetrahydropyran-2-yl]methyl acetate (15-3) (2.500 g, 40%) as a yellow solid.

1H-NMR (500 MHz, DMSO, 25° C.) δ 1.24 (m, J=14.4, 9.8, 7.4 Hz, 12H), 1.38 (m, J=22.0, 14.0, 6.9 Hz, 13H), 1.46 (d, J=6.7 Hz, 4H), 1.77 (s, 9H), 1.89 (s, 9H), 1.99 (s, 9H), 2.07 (s, 2H), 2.10 (s, 9H), 2.23-2.35 (m, 6H), 3.03 (m, J=18.4, 11.9, 5.4 Hz, 10H), 3.40 (m, J=10.0, 6.6 Hz, 3H), 3.70 (m, J=17.6, 11.5, 4.4 Hz, 10H), 3.87 (m, J=11.3, 8.9 Hz, 3H), 4.02 (q, J=3.9 Hz, 10H), 4.48 (d, J=8.5 Hz, 3H), 4.94-5.02 (m, 5H), 5.21 (d, J=3.4 Hz, 3H), 6.28 (s, 1H), 7.23 (t, J=5.6 Hz, 1H), 7.28-7.4 (m, 5H), 7.73-7.87 (m, 7H). LCMS m/z Expected 1878.9, Observed 940.7 (M+1, z=2).

TMS-I (0.724 mL, 5.32 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[4-(benzyloxycarbonylamino)butylcarbamoyl]cyclohex-3-en-1-yl]oxypropanoylamino]hexoxy]tetrahydropyran-2-yl]methyl acetate (15-3) (1 g, 0.53 mmol) in MeCN (15 mL) at RT under nitrogen. The resulting mixture was stirred at RT for 15 minutes. The solvent was removed under reduced pressure. DCM (50 ml×4) was added to the residue and concentrated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(4-aminobutylcarbamoyl)cyclohex-3-en-1-yl]oxypropanoylamino]hexoxy]tetrahydropyran-2-yl]methyl acetate (15-4) (1.000 g, 108%) as a yellow solid.

1H-NMR (500 MHz, DMSO, 25° C.) δ 1.19-1.28 (m, 12H), 1.29-1.44 (m, 12H), 1.46-1.54 (m, 6H), 1.77 (d, J=1.7 Hz, 5H), 1.80 (s, 4H), 1.89 (d, J=1.9 Hz, 9H), 1.99 (d, J=1.2 Hz, 9H), 2.10 (s, 9H), 2.22-2.4 (m, 8H), 2.78 (m, J=5.9 Hz, 2H), 3.02 (m, J=6.4 Hz, 6H), 3.37-3.41 (m, 2H), 3.56-3.89 (m, 13H), 3.98-4.01 (m, 2H), 4.01-4.06 (m, 6H), 4.11-4.23 (m, 3H), 4.98 (m, J=21.2, 11.6, 3.4 Hz, 3H), 5.19-5.32 (m, 3H), 5.93 (s, 6H), 6.28 (s, 1H), 7.74-7.95 (m, 6H), 7.99 (d, J=8.1 Hz, 1H). LCMS m/z Expected 1744.9, Observed 873.8 (m+1, z=2).

DIEA (0.500 mL, 2.86 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(4-aminobutylcarbamoyl)cyclohex-3-en-1-yl]oxypropanoylamino]hexoxy]tetrahydropyran-2-yl]methyl acetate (15-4) (1 g, 0.57 mmol), O-(Benzotriazol-1-yl)-N—N—N′,N′-tetramethyluronium hexafluorophosphate (0.652 g, 1.72 mmol) and adipic acid (0.419 g, 2.86 mmol) in DMF (20 mL) at RT under nitrogen. The resulting mixture was stirred at RT for 2 hours. The solvent was removed under reduced pressure to afford the crude product. The crude product was purified by flash C18-flash chromatography (130 g), elution gradient 0 to 40% MeCN in water (0.1% TFA). Pure fractions were evaporated to dryness to afford 6-oxo-6-((4-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-ene-1-carboxamido)butyl)amino)hexanoic acid (15-5) (0.333 g, 31%) as a white solid.

1H-NMR (500 MHz, DMSO, 25° C.) δ 1.2-1.29 (m, 10H), 1.37 (m, J=12.8, 10.8, 4.3 Hz, 12H), 1.42-1.49 (m, 8H), 1.53 (m, J=11.8, 9.7, 4.9 Hz, 3H), 1.77 (s, 5H), 1.81 (s, 4H), 1.89 (d, J=1.5 Hz, 9H), 1.99 (d, J=1.2 Hz, 9H), 2.04 (t, J=6.9 Hz, 2H), 2.10 (s, 10H), 2.17-2.4 (m, 10H), 3.03 (m, J=11.3, 5.7, 5.2 Hz, 10H), 3.36-3.44 (m, 3H), 3.55-3.6 (m, 1H), 3.72 (m, J=12.6, 4.7 Hz, 9H), 3.87 (m, J=11.3, 8.8 Hz, 2H), 3.99-4.05 (m, 8H), 4.14 (t, J=6.7 Hz, 1H), 4.20 (m, J=11.7, 8.2, 3.5 Hz, 1H), 4.48 (d, J=8.5 Hz, 2H), 4.83 (d, J=3.5 Hz, 1H), 4.99 (m, J=21.7, 11.5, 3.4 Hz, 3H), 5.21 (d, J=3.4 Hz, 2H), 5.32 (m, J=3.3, 1.2 Hz, 1H), 6.28 (s, 1H), 7.72-7.78 (m, 2H), 7.8-7.86 (m, 4H), 7.99 (m, J=8.7, 3.4 Hz, 2H). LCMS m/z Expected 1872.9, Observed 937.6 (M+1, z=2).

Perfluorophenyl 2,2,2-trifluoroacetate (0.083 mL, 0.48 mmol) was added to (15-5) (150 mg, 0.08 mmol) and DIEA (0.14 mL, 0.8 mmol) in DCM (2 mL) at RT. The resulting mixture was stirred at RT for 4 h. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (15) (155 mg, 95%) as white solid. The product was used in the next reaction without any further purification. LCMS m/z Expected 2038.9, Observed 1020.9 [M+H]+(z=2).

### Synthesis of Precursor (16)

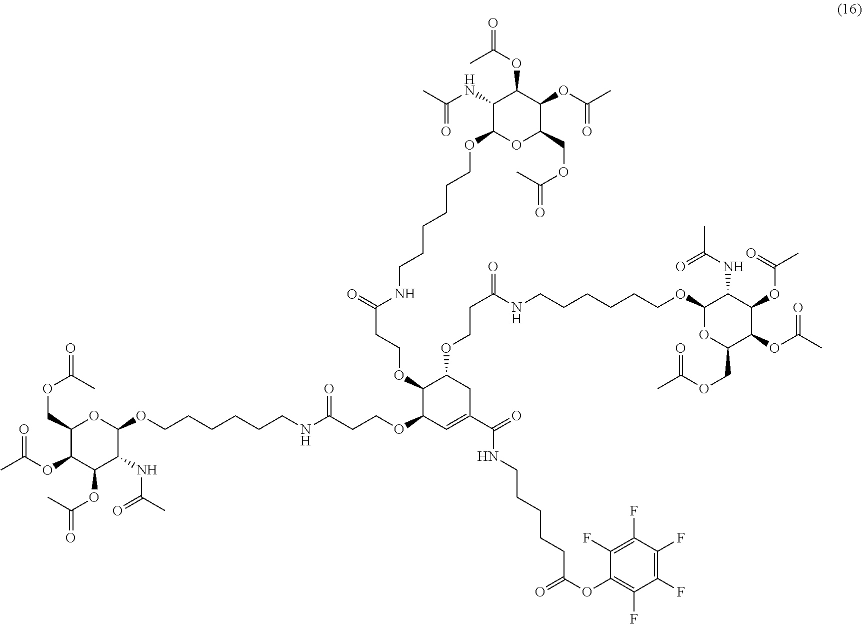 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)NCCCCCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

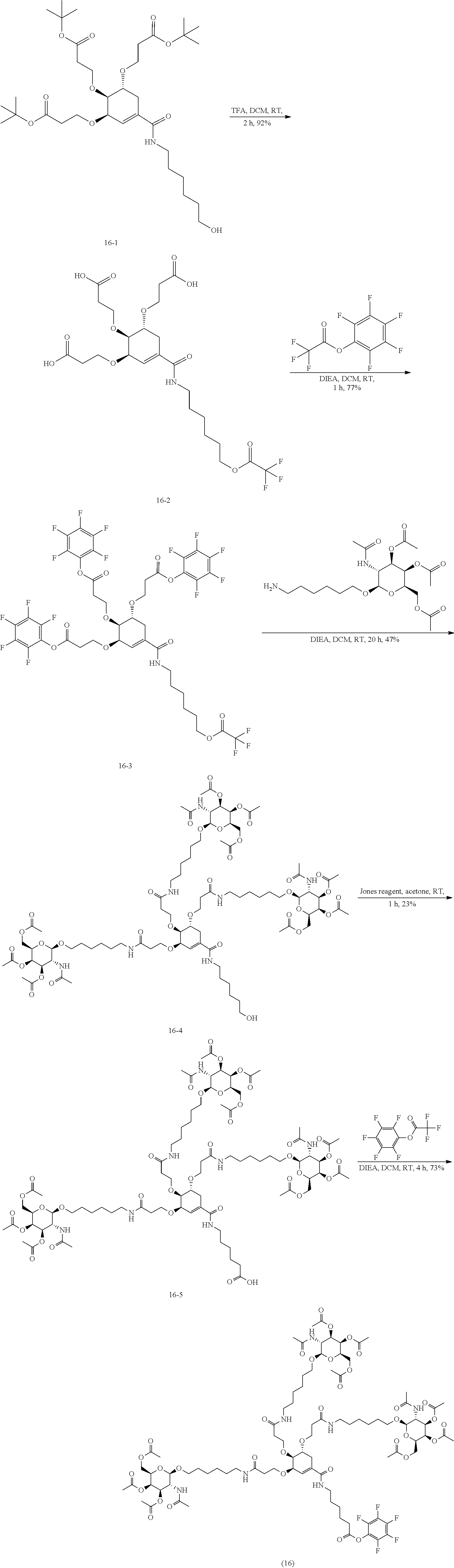

TFA (9.22 mL, 119.71 mmol) was added to tri-tert-butyl 3,3′,3″-(((1R,2S,3R)-5-((6-hydroxyhexyl)carbamoyl)cyclohex-4-ene-1,2,3-triyl)tris(oxy))tripropionate (16-1) (3.15 g, 4.79 mmol) in DCM (20 mL) at 0° C. The resulting mixture was stirred at RT for 2 hours. The solvent was removed under reduced pressure. DCM (3×50 mL) was added to the residue and concentrated to dryness to afford 3,3′,3″-(((1R,2S,3R)-5-((6-(2,2,2-trifluoroacetoxy)hexyl) carbamoyl)cyclohex-4-ene-1,2,3-triyl)tris(oxy))tripropionic acid (16-2) (2.58 g, 92%) as a colorless oil. The product was used in the next step directly without further purification.

1H-NMR (500 MHz, DMSO, 25° C.) δ 1.27-1.30 (m, 6H), 1.40-1.43 (m, 2H), 1.51-1.62 (m, 1H), 1.62-1.75 (m, 2H), 2.14 (dd, J=18.3, 3.7 Hz, 1H), 2.31-2.49 (m, 6H), 2.98-3.26 (m, 2H), 3.57-3.85 (m, 7H), 4.37 (t, J=6.6 Hz, 2H), 6.26 (d, J=2.7 Hz, 1H), 7.88 (q, J=5.7 Hz, 1H). Three protons have been exchanged. LCMS m/z Expected 585.2, Observed 586.2.

Perfluorophenyl 2,2,2-trifluoroacetate (3.37 mL, 19.60 mmol) was added to 3,3′,3″-(((1R,2S,3R)-5-((6-(2,2,2-trifluoroacetoxy)hexyl)carbamoyl)cyclohex-4-ene-1,2,3-triyl)tris(oxy))tripropionic acid (16-2) (2.55 g, 4.36 mmol) and DIEA (5.70 mL, 32.66 mmol) in DCM (50 mL) at RT. The resulting mixture was stirred at RT for 1 hour. The reaction mixture was diluted with DCM (40 ml) and washed sequentially with 1M NaHSO4 (40 ml), saturated NaHCO₃ (50 ml), and saturated brine (50 ml). The organic layer was dried over Na2SO4, filtered and evaporated to afford tris(perfluorophenyl) 3,3′,3″-(((1R,2S,3R)-5-((6-(2,2,2-trifluoroacetoxy)hexyl)carbamoyl)cyclohex-4-ene-1,2,3-triyl)tris(oxy))tripropionate (16-3) (3.65 g, 77%) as a yellow oil. The product was used in the next step directly without further purification.

1H-NMR (500 MHz, DMSO, 25° C.) δ 1.32-1.35 (m, 4H), 1.42-1.45 (m, 3H), 1.59-1.73 (m, 3H), 2.14-2.27 (m, 1H), 2.38-2.47 (m, 1H), 3.05-3.21 (m, 4H), 3.67-4.03 (m, 10H), 4.36 (t, J=6.6 Hz, 3H), 6.34 (s, 1H), 7.88 (t, J=5.8 Hz, 1H). LCMS m/z Expected 1083.1, Observed 1084.2.

DIEA (5.63 mL, 32.24 mmol) was added to (2R,3R,4R,5R,6R)-5-acetamido-2-(acetoxymethyl)-6-((6-aminohexyl)oxy)tetrahydro-2H-pyran-3,4-diyl diacetate (5.76 g, 12.89 mmol) and tris(perfluorophenyl) 3,3′,3″-(((1R,2S,3R,5S)-5-((6-(2,2,2-trifluoroacetoxy)hexyl) carbamoyl)cyclohexane-1,2,3-triyl)tris(oxy))tripropionate (16-3) (3.5 g, 3.22 mmol) in DMF (100 mL) at 0° C. The resulting mixture was stirred at RT for 2 hours. The solvent was removed under reduced pressure to afford the crude product. The crude product was purified by flash C18-flash chromatography, elution gradient 0 to 50% MeCN in water (0.05% TFA). Pure fractions were evaporated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(6-hydroxyhexylcarbamoyl)cyclohex-3-en-1-yl]oxypropanoylamino]hexoxy]tetrahydropyran-2-yl]methyl acetate (16-4) (2.70 g, 47%) as a white solid.

1H-NMR (500 MHz, DMSO, 25° C.) δ 1.14-1.31 (m, 17H), 1.31-1.52 (m, 17H), 1.77 (s, 9H), 1.89 (s, 9H), 1.99 (s, 9H), 2.10 (s, 10H), 2.18-2.43 (m, 7H), 3.03-3.22 (m, 8H), 3.29-3.45 (m, 6H), 3.74 (d, J=6.9 Hz, 8H), 3.87 (dt, J=11.3, 8.8 Hz, 4H), 4.02 (q, J=3.9 Hz, 10H), 4.48 (d, J=8.5 Hz, 3H), 4.97 (dd, J=11.3, 3.5 Hz, 3H), 5.21 (d, J=3.4 Hz, 3H), 6.27 (s, 1H), 7.67-7.98 (m, 7H). LCMS m/z Expected 1773.8, Observed 888.3 (z=2).

Chromosulfuric acid (0.732 mL, 1.46 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-(6-hydroxyhexylcarbamoyl)cyclohex-3-en-1-yl]oxypropanoylamino]hexoxy]tetrahydropyran-2-yl]methyl acetate (16-4) (1.3 g, 0.73 mmol) in acetone (35 mL) at 0° C. The resulting mixture was stirred at RT for 1 hour. The reaction mixture was quenched with i-PrOH (20 ml). The mixture was diluted with DCM (30 ml), after 10 min, saturated NaHCO₃ (50 ml) was added. The solution was evaporated to dryness to afford the crude product. The crude product was purified by flash C18-flash chromatography, elution gradient 0 to 30% MeCN in water (0.05% TFA). Pure fractions were evaporated to dryness to afford 6-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-ene-1-carboxamido)hexanoic acid (16-5) (0.308 g, 23%) as a yellow solid.

1H-NMR (500 MHz, DMSO, 25° C.) δ 1.25-1.27 (m, 17H), 1.28-1.32 (m, 6H), 1.38-1.52 (m, 9H), 1.77 (s, 9H), 1.89 (s, 9H), 1.99 (s, 9H), 2.10 (s, 9H), 2.18-2.42 (m, 8H), 3.02-3.12 (m, 8H), 3.28-3.42 (m, 5H), 3.70-3.82 (m, 10H), 3.86 (q, J=9.6 Hz, 3H), 4.02 (q, J=6.3, 5.3 Hz, 9H), 4.48 (d, J=8.4 Hz, 3H), 4.96 (dd, J=11.2, 3.4 Hz, 3H), 5.21 (d, J=3.4 Hz, 3H), 6.27 (s, 1H), 7.68-7.82 (mz, 6H), 11.95 (brs, 1H). One proton has been exchanged. LCMS m/z Expected 1787.8, Observed 895.1 (z=2).

Perfluorophenyl 2,2,2-trifluoroacetate (0.12 mL, 0.7 mmol) was added to (16-5) (178 mg, 0.1 mmol) and DIEA (0.174 mL, 0.99 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (16) (142 mg, 73%) as beige solid. The product was used in the next reaction without any further purification. LCMS m/z Expected 1953.8, Observed 978.3 [M+H]+(z=2).

### Synthesis of Precursor (17)

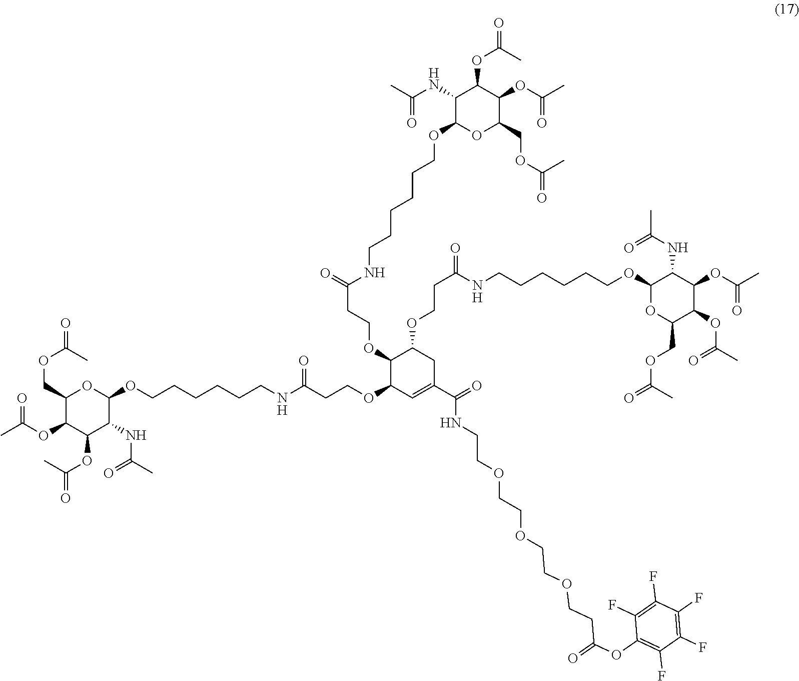 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)NCCOCCOCCOCCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

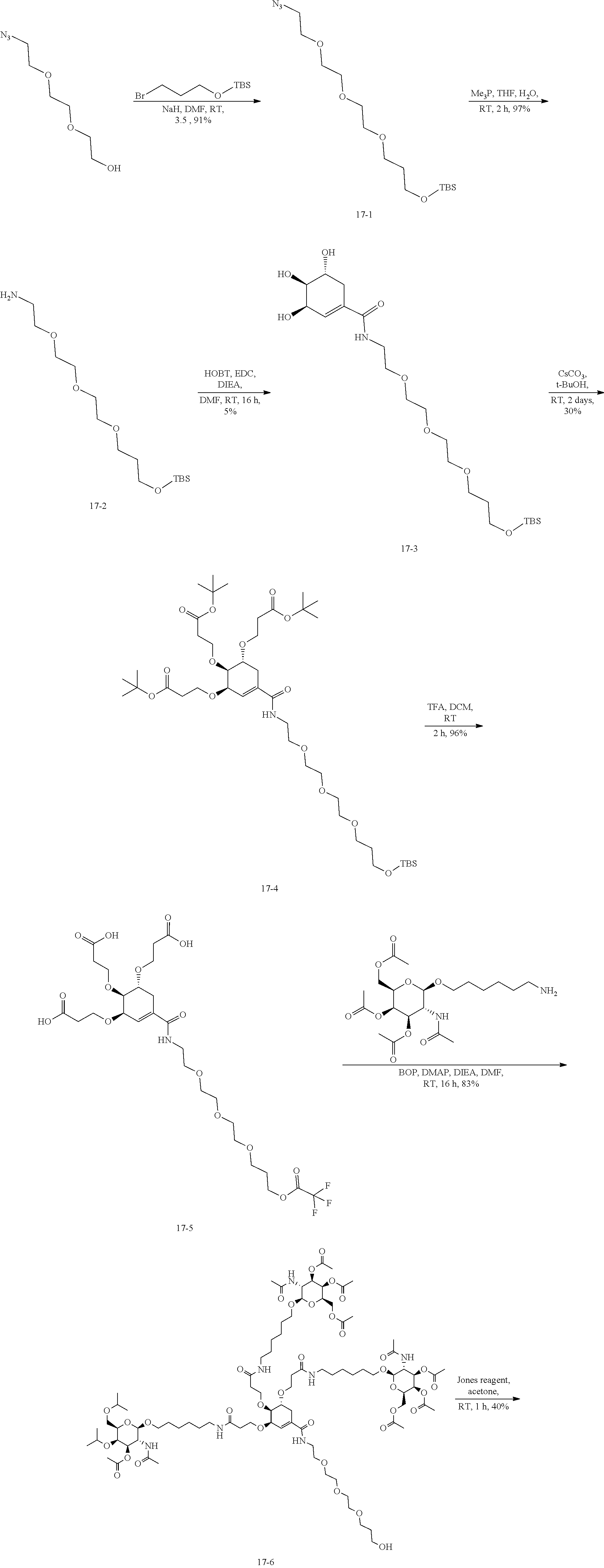

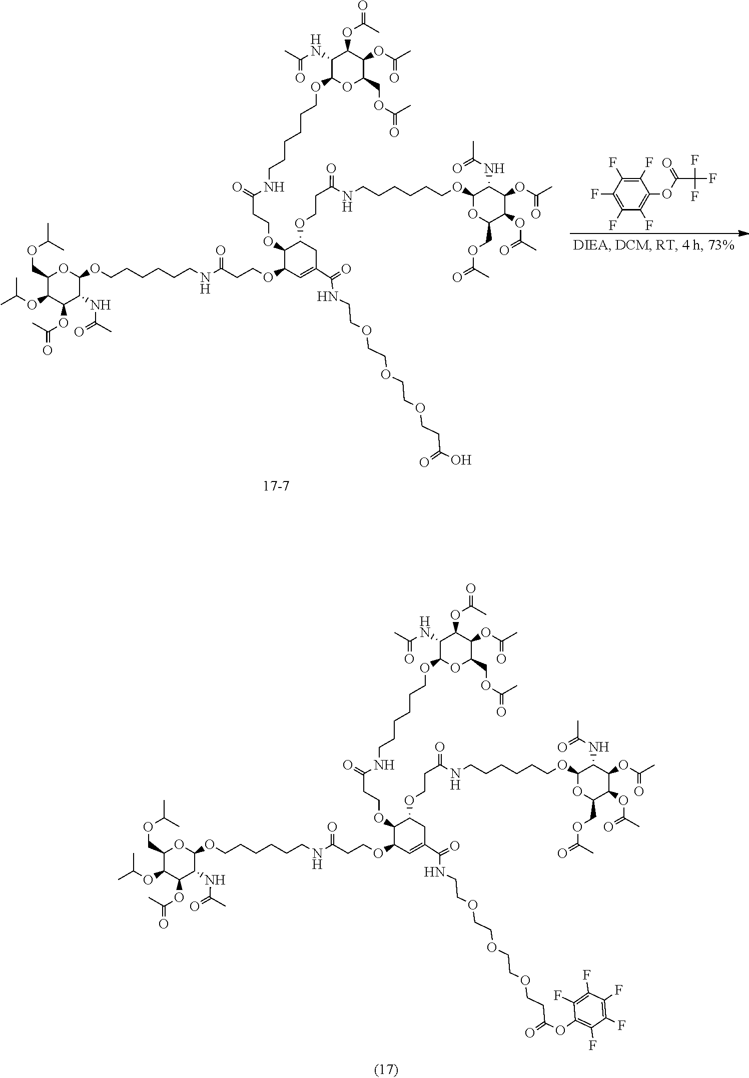

Sodium hydride (60%) (5.71 g, 142.70 mmol) was added slowly to 2-(2-(2-azidoethoxy)ethoxy)ethan-1-ol (10 g, 57.08 mmol) in DMF (200 mL) at 0° C. under nitrogen. After stirring for 1.5 h, (3-bromopropoxy)(tert-butyl)dimethylsilane (28.9 g, 114.16 mmol) was added to the mixture. The resulting mixture was stirred at RT for 2 hours. The reaction mixture was quenched with cold water (50 mL), extracted with EtOAc (2×150 mL). The organic layer was washed with saturated brine (2×250 mL), dried over Na2SO4, filtered and evaporated to afford 16-azido-2,2,3,3-tetramethyl-4,8,11,14-tetraoxa-3-silahexadecane (17-1) (18.00 g, 91%) as a yellow oil. The product was used in the next step directly without further purification.

1H NMR (300 MHz, DMSO, 24° C.) δ 0.03 (d, J=3.0 Hz, 6H), 0.86 (d, J=3.1 Hz, 10H), 1.1-1.27 (m, 1H), 1.67 (p, J=6.3 Hz, 2H), 3.35-3.46 (m, 4H), 3.5-3.56 (m, 6H), 3.57-3.67 (m, 4H). LCMS m/z Expected 347.2, Observed 348.2.

Trimethylphosphine (86 mL, 86.32 mmol) was added to 16-azido-2,2,3,3-tetramethyl-4,8,11,14-tetraoxa-3-silahexadecane (17-1) (10 g, 28.77 mmol) and H₂O (10.37 mL, 575.49 mmol) in THE (200 mL) at RT. The resulting mixture was stirred at RT for 2 hours. The solvent was removed under reduced pressure to afford 2,2,3,3-tetramethyl-4,8,11,14-tetraoxa-3-silahexadecan-16-amine (17-2) (9.00 g, 97%) as a colorless oil. The product was used in the next step directly without further purification.

1H NMR (300 MHz, DMSO, 22° C.) δ 0.01 (s, 6H), 0.85 (s, 9H), 1.34 (s, 1H), 1.38 (s, 1H), 1.65 (t, J=6.3 Hz, 2H), 2.62 (t, J=5.8 Hz, 2H), 3.33 (t, J=5.8 Hz, 2H), 3.39-3.55 (m, 10H), 3.62 (t, J=6.2 Hz, 2H). LCMS m/z Expected 321.2, Observed 322.2.

DIEA (15.04 mL, 86.13 mmol) was added to 2,2,3,3-tetramethyl-4,8,11,14-tetraoxa-3-silahexadecan-16-amine (17-2) (8.77 g, 27.28 mmol), (3R,4S,5R)-3,4,5-trihydroxycyclohex-1-ene-1-carboxylic acid (5 g, 28.71 mmol), EDC (11.01 g, 57.42 mmol) and HOBt (8.79 g, 57.42 mmol) in DMF (100 mL) at RT under nitrogen. The resulting mixture was stirred at RT for 3 hours. The solvent was removed under reduced pressure to afford crude product. The crude product was purified by flash C18-flash chromatography, elution gradient 0 to 60% MeCN in water (0.01% FA). The product was repurified by flash silica chromatography, elution gradient 0 to 20% MeOH in DCM. Pure fractions were evaporated to dryness to afford (3R,4S,5R)-3,4,5-trihydroxy-N-(2,2,3,3-tetramethyl-4,8,11,14-tetraoxa-3-silahexadecan-16-yl)cyclohex-1-ene-1-carboxamide (17-3) (0.700 g, 5%) as a colorless oil.

1H NMR (300 MHz, DMSO, 22° C.) δ 0.01 (s, 6H), 0.84 (s, 9H), 1.6-1.7 (m, 2H), 1.88-2.02 (m, 1H), 3.19-3.27 (m, 2H), 3.37-3.52 (m, 14H), 3.61 (t, J=6.2 Hz, 2H), 3.75-3.84 (m, 1H), 4.1-4.22 (m, 1H), 4.51 (d, J=4.4 Hz, 1H), 4.66 (d, J=6.9 Hz, 1H), 4.75 (d, J=3.8 Hz, 1H), 6.27 (d, J=3.2, 1.7 Hz, 1H), 7.81 (t, J=5.6 Hz, 1H). LCMS m/z Expected 477.3, Observed 478.3.

Tert-Butyl acrylate (23.92 mL, 163.29 mmol) was added to (3R,4S,5R)-3,4,5-trihydroxy-N-(2,2,3,3-tetramethyl-4,8,11,14-tetraoxa-3-silahexadecan-16-yl)cyclohex-1-ene-1-carboxamide (17-3) (1.3 g, 2.72 mmol) and Cs₂CO₃ (2.93 g, 8.98 mmol) in t-BuOH (100 mL) at RT. The resulting mixture was stirred at RT for 2 days. The reaction mixture was filtered through celite, washed with EtOAc (500 mL) and dried under vacuum to afford crude product. The crude product was purified by flash silica chromatography, elution gradient 0 to 70% EtOAc in petroleum ether (product runs at 70%). Pure fractions were evaporated to dryness to afford tri-tert-butyl 3,3′,3″-(((1R,2S,3R)-5-((2,2,3,3-tetramethyl-4,8,11,14-tetraoxa-3-silahexadecan-16-yl)carbamoyl)cyclohex-4-ene-1,2,3-triyl)tris(oxy))tripropionate (17-4) (0.700 g, 30%) as a colorless oil.

1H NMR (300 MHz, DMSO, 22° C.) δ 0.01 (s, 6H), 0.84 (s, 9H), 1.34-1.41 (m, 27H), 1.54-1.71 (m, 2H), 2.28-2.45 (m, 7H), 3.17-3.27 (m, 2H), 3.32-3.55 (m, 14H), 3.57-3.77 (m, 9H), 4.03 (s, 1H), 6.30 (s, 1H), 7.90 (t, J=5.6 Hz, 1H). LCMS m/z Expected 861.5, Observed 862.5.

TFA (10.00 mL) was added to tri-tert-butyl 3,3′,3″-(((1R,2S,3R)-5-((2,2,3,3-tetramethyl-4,8,11,14-tetraoxa-3-silahexadecan-16-yl)carbamoyl)cyclohex-4-ene-1,2,3-triyl)tris(oxy))tripropionate (17-4) (1 g, 1.16 mmol) in DCM (20.000 mL) at RT. The resulting mixture was stirred at RT for 2 hours. The solvent was removed under reduced pressure to afford tri-tert-butyl 3,3′,3″-(((1R,2S,3R)-5-((2,2,3,3-tetramethyl-4,8,11,14-tetraoxa-3-silahexadecan-16-yl)carbamoyl)cyclohex-4-ene-1,2,3-triyl)tris(oxy))tripropionate (17-5) (1 g, 1.16 mmol) as a yellow oil. The product was used in the next step directly without further purification.

1H NMR (300 MHz, DMSO, 24° C.) δ 1.87-1.97 (m, 2H), 2.35-2.48 (m, 7H), 3.18-3.28 (m, 2H), 3.43-3.52 (m, 13H), 3.68-3.8 (m, 8H), 4.08 (s, 1H), 4.44 (t, J=6.5 Hz, 2H), 6.29 (d, J=2.4 Hz, 1H), 7.94 (t, J=5.6 Hz, 1H). LCMS m/z Expected 675.2, Observed 676.1.

DIEA (1.810 mL, 10.36 mmol) was added to 3,3′,3″-(((1R,2S,3R)-5-((15,15,15-trifluoro-14-oxo-3,6,9,13-tetraoxapentadecyl)carbamoyl)cyclohex-4-ene-1,2,3-triyl)tris(oxy))tripropionic acid (17-5) (700 mg, 1.04 mmol), (2R,3R,4R,5R,6R)-5-acetamido-2-(acetoxymethyl)-6-((6-aminohexyl)oxy)tetrahydro-2H-pyran-3,4-diyl diacetate (2036 mg, 4.56 mmol), BOP (1.84 g, 4.14 mmol) and DMAP (380 mg, 3.11 mmol) in DMF (15 mL) at RT under nitrogen. The resulting mixture was stirred at RT for 2 hours. The solvent was removed under reduced pressure to afford crude product. The crude product was purified by flash C18-flash chromatography, elution gradient 0 to 60% MeCN in water (0.1% TFA). Pure fractions were evaporated to dryness to afford [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[2-[2-[2-(3-hydroxypropoxy)ethoxy]ethoxy]ethylcarbamoyl]cyclohex-3-en-1-yl]oxypropanoylamino]hexoxy]tetrahydropyran-2-yl]methyl acetate (17-6) (1.6 g, 83%) as a yellow solid

1H NMR (300 MHz, DMSO, 23° C.) δ 1.25 (s, 18H), 1.36 (s, 1H), 1.46 (s, 7H), 1.57-1.68 (m, 2H), 1.77 (s, 9H), 1.90 (s, 9H), 2.00 (s, 9H), 2.09 (d, J=8.1 Hz, 14H), 2.2-2.37 (m, 7H), 3.02 (s, 6H), 3.11-3.29 (m, 3H), 3.34-3.55 (m, 19H), 3.59-3.79 (m, 11H), 3.8-3.94 (m, 4H), 4.49 (d, J=8.4 Hz, 3H), 4.97 (dd, J=11.2, 3.4 Hz, 3H), 5.22 (d, J=3.4 Hz, 3H), 6.31 (s, 1H), 7.7-7.94 (m, 7H). One proton has been exchanged. LCMS m/z Expected 1863.4, Observed 1865.1.

Chromosulfuric acid (0.483 mL, 0.97 mmol) was added to [(2R,3R,4R,5R,6R)-5-acetamido-3,4-diacetoxy-6-[6-[3-[(1R,5R,6S)-5,6-bis[3-[6-[(2R,3R,4R,5R,6R)-3-acetamido-4,5-diacetoxy-6-(acetoxymethyl)tetrahydropyran-2-yl]oxyhexylamino]-3-oxo-propoxy]-3-[2-[2-[2-(3-hydroxypropoxy)ethoxy]ethoxy]ethylcarbamoyl]cyclohex-3-en-1-yl]oxypropanoylamino]hexoxy]tetrahydropyran-2-yl]methyl acetate (17-6) (0.9 g, 0.48 mmol) in acetone (15 mL) at 0° C. The resulting mixture was stirred at RT for 1 hour. The reaction mixture was quenched with i-PrOH (10 ml), diluted with DCM (10 ml). After stirring for 10 min, saturated NaHCO₃ (20 ml) was added. The solution was evaporated to dryness to afford crude product. The crude product was purified by flash C18-flash chromatography, elution gradient 0 to 40% MeCN in water (0.1% TFA). Pure fractions were evaporated to dryness to afford 1-oxo-1-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-en-1-yl)-5,8,11-trioxa-2-azatetradecan-14-oic acid (17-7) (370 mg, 40%) as a white solid.

1H NMR (500 MHz, DMSO, 25° C.) δ 1.25 (d, J=10.0 Hz, 12H), 1.31-1.4 (m, 6H), 1.41-1.49 (m, 6H), 1.77 (s, 9H), 1.89 (s, 9H), 1.99 (s, 9H), 2.10 (s, 9H), 2.12-2.41 (m, 10H), 2.98-3.04 (m, 5H), 3.24 (q, J=5.8 Hz, 2H), 3.37-3.46 (m, 8H), 3.49 (d, J=10.1 Hz, 6H), 3.53-3.6 (m, 3H), 3.63-3.78 (m, 10H), 3.83-3.91 (m, 3H), 3.97-4.05 (m, 10H), 4.51 (d, J=8.5 Hz, 3H), 4.98 (dd, J=11.2, 3.4 Hz, 3H), 5.22 (d, J=3.0 Hz, 3H), 6.33 (s, 1H), 7.72-8.11 (m, 7H). LCMS m/z Expected 1877.9, Observed 940.4 (z=2).

Perfluorophenyl 2,2,2-trifluoroacetate (0.064 mL, 0.37 mmol) was added to (17-7) (100 mg, 0.05 mmol) and DIEA (0.093 mL, 0.53 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (17) (79 mg, 73%) as white solid. The product was used in the next reaction without any further purification. LCMS m/z Expected 2043.8, Observed 1023.4 [M+H]+(z=2).

### Synthesis of Precursor (18)

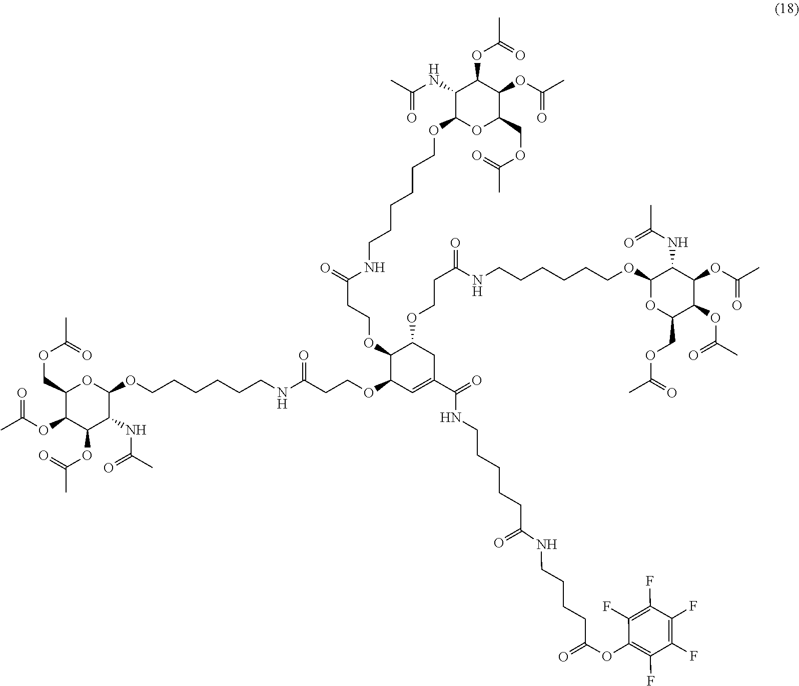 [SMILES: CC(=O)N[C@H]1[C@H](OCCCCCCNC(=O)CCO[C@@H]2[C@H](OCCC(=O)NCCCCCCO[C@@H]3O[C@H](COC(C)=O)[C@H](OC(C)=O)[C@H](OC(C)=O)[C@H]3NC(C)=O)C=C(C(=O)NCCCCCC(=O)NCCCCC(=O)Oc3c(F)c(F)c(F)c(F)c3F)C[C@H]2OCCC(=O)NCCCCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

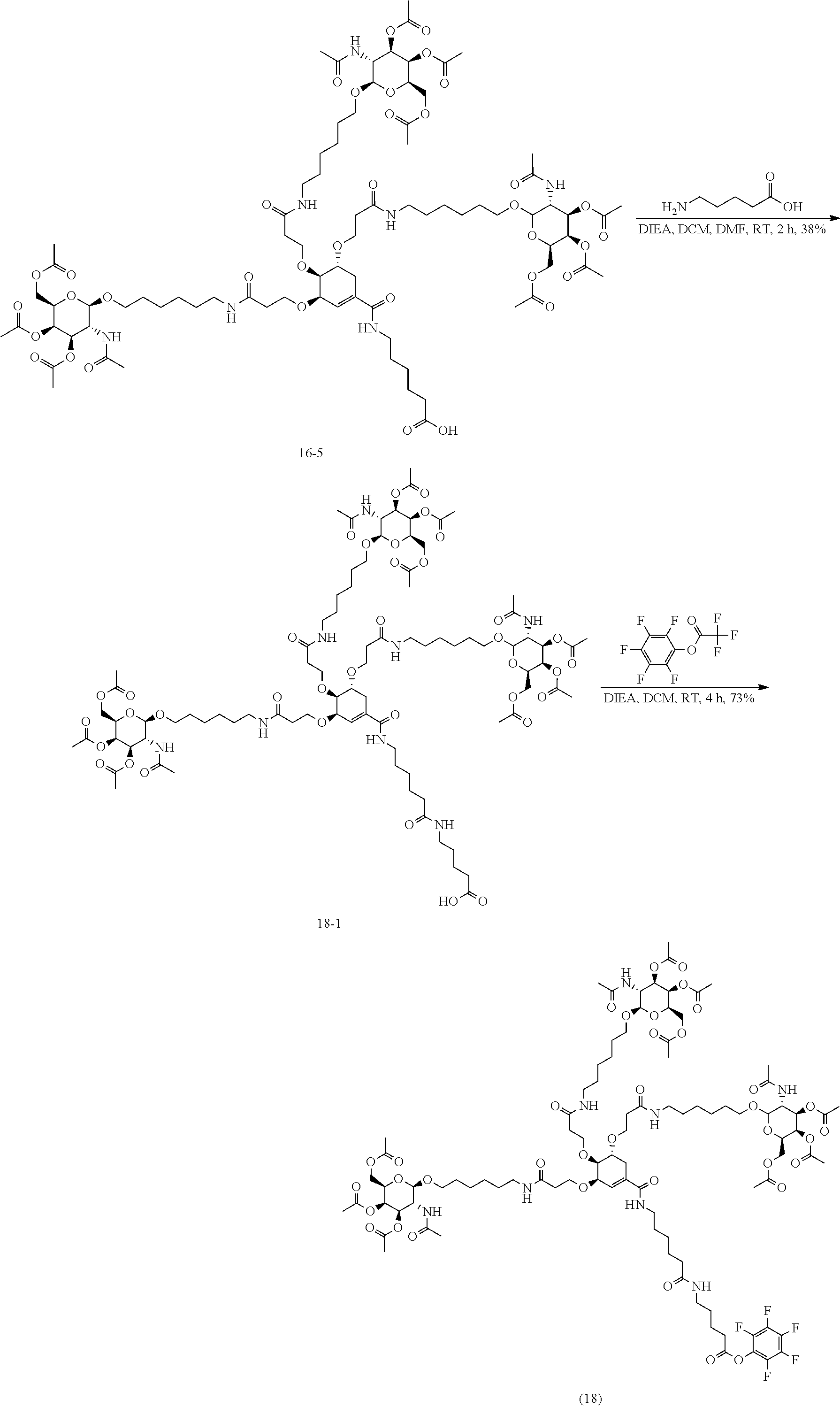

Pentafluorophenyl trifluoroacetate (0.069 mL, 0.40 mmol) was added to 6-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-ene-1-carboxamido)hexanoic acid (16-5) (480 mg, 0.27 mmol) and DIEA (0.351 mL, 2.01 mmol) in DCM (8 mL). The resulting mixture was stirred at RT for 1 hour. A solution of 5-aminovaleric acid (62.9 mg, 0.54 mmol) in DMF (8.00 mL) was added to the mixture at RT, then the reaction mixture was stirred at RT for 2 hours. The reaction was quenched with water (2 ml). The solvent was removed under reduced pressure to afford the crude product. The crude product was purified by C18-flash chromatography, elution gradient 0 to 30% MeCN in water (0.05% TFA). Pure fractions were evaporated to dryness to afford 5-(6-((3R,4S,5R)-3,4,5-tris(3-((6-(((2R,3R,4R,5R,6R)-3-acetamido-4,5-diacetoxy-6-(acetoxymethyl)tetrahydro-2H-pyran-2-yl)oxy)hexyl)amino)-3-oxopropoxy)cyclohex-1-ene-1-carboxamido)hexanamido)pentanoic acid (18-1) (193 mg, 38%) as a white solid.

1H NMR (300 MHz, DMSO, 24° C.) δ 1.24-1.29 (m, 15H), 1.42-1.50 (m, 20H), 1.77 (s, 9H), 1.90 (s, 9H), 2.00 (s, 10H), 2.11 (s, 10H), 2.16-2.4 (m, 9H), 3.04 (h, J=6.5, 6.1 Hz, 10H), 3.41 (dt, J=9.7, 6.4 Hz, 3H), 3.55-3.79 (m, 11H), 3.87 (dt, J=11.2, 8.8 Hz, 3H), 4.03 (d, J=12.7 Hz, 10H), 4.49 (d, J=8.4 Hz, 3H), 4.97 (dd, J=11.2, 3.4 Hz, 3H), 5.22 (d, J=3.4 Hz, 3H), 6.28 (s, 1H), 7.79 (dd, J=21.9, 8.7 Hz, 8H). LCMS m/z Expected 1886.9, Observed 944.8 (z=2).

Perfluorophenyl 2,2,2-trifluoroacetate (0.064 mL, 0.37 mmol) was added to (18-1) (100 mg, 0.05 mmol) and DIEA (0.093 mL, 0.53 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (18) (103 mg, 95%) as white solid. The product was used in the next reaction without any further purification. LCMS m/z Expected 2052.9, Observed 1027.8 [M+H]+(z=2).

### Synthesis of Precursor (19)

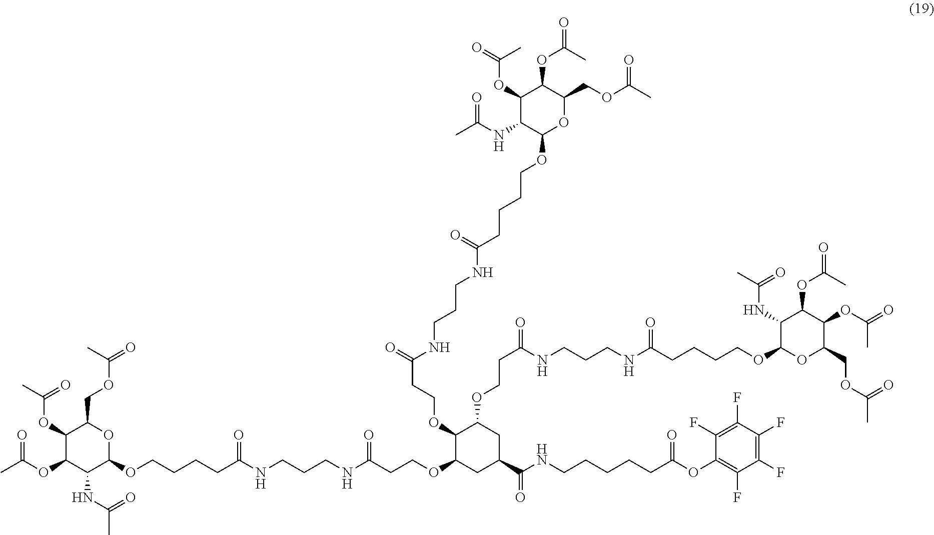 [SMILES: CC(=O)N[C@H]1[C@H](OCCCCC(=O)NCCCNC(=O)CCO[C@@H]2C[C@H](C(=O)NCCCCCC(=O)Oc3c(F)c(F)c(F)c(F)c3F)C[C@@H](OCCC(=O)NCCCNC(=O)CCCCO[C@@H]3O[C@H](COC(C)=O)[C@H](OC(C)=O)[C@H](OC(C)=O)[C@H]3NC(C)=O)[C@@H]2OCCC(=O)NCCCNC(=O)CCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

### Synthetic Scheme:

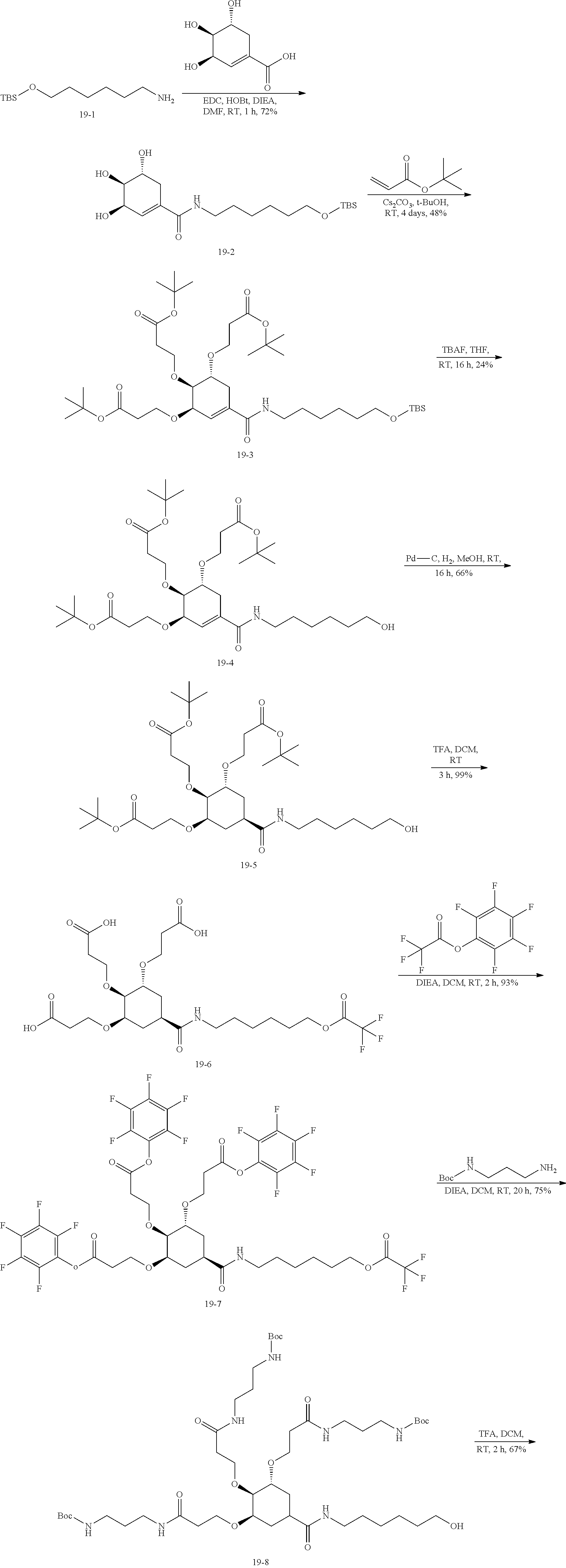

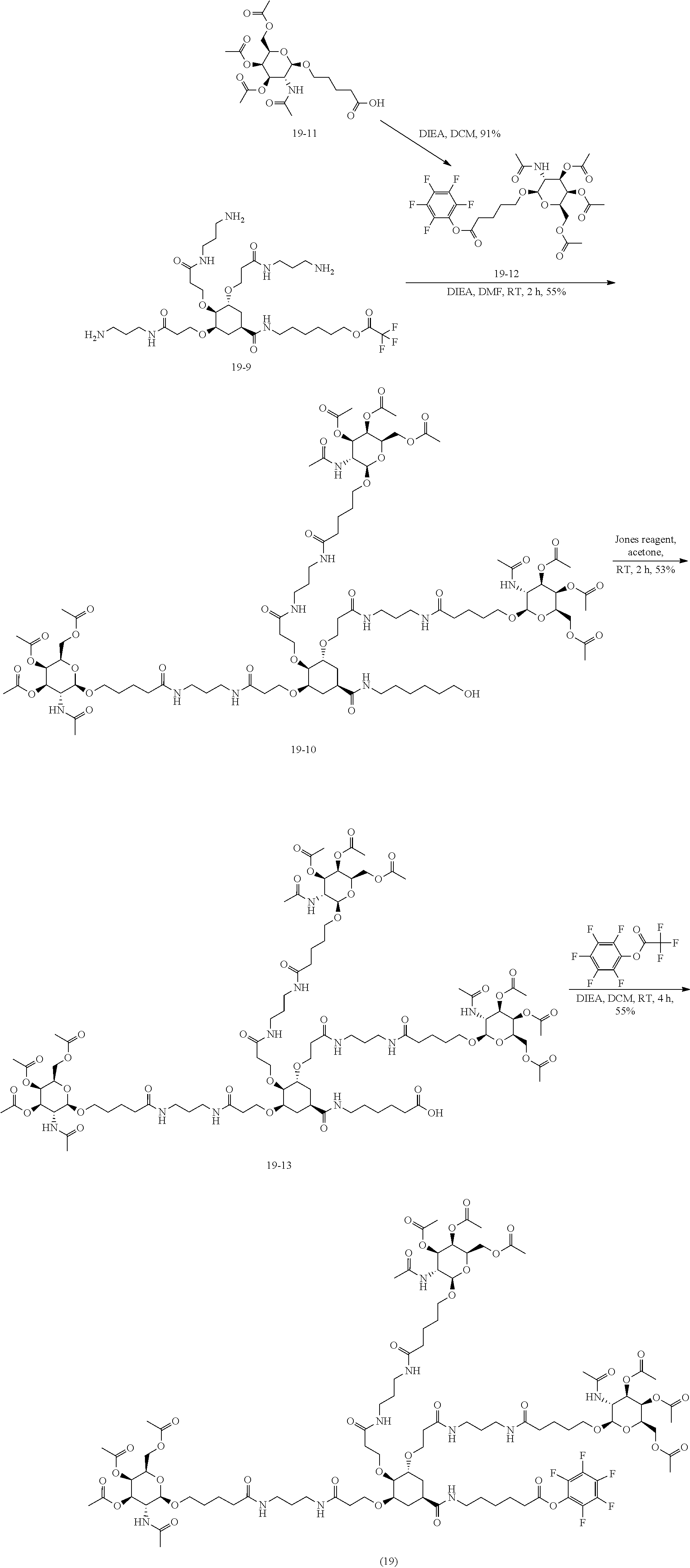

6-((tert-butyldimethylsilyl)oxy)hexan-1-amine (5 g, 21.60 mmol) was added to (3R,4S,5R)-3,4,5-trihydroxycyclohex-1-ene-1-carboxylic acid (4.14 g, 23.76 mmol), EDC (8.70 g, 45.37 mmol), HOBt (6.62 g, 43.20 mmol) and DIEA (11.32 mL, 64.81 mmol) in DMF (50 mL). The resulting mixture was stirred at rt for 1 hour. The solvent was removed under reduced pressure to afford the crude product. The crude product was purified by flash silica chromatography, elution gradient 0 to 5% McOH in DCM. Pure fractions were evaporated to dryness to afford (3R,4S,5R)—N-(6-((tert-butyldimethylsilyl)oxy)hexyl)-3,4,5-trihydroxycyclohex-1-ene-1-carboxamide (19-2) (4.40 g, 72%) as a yellow liquid.

1H NMR (400 MHz, DMSO, 24° C.) δ 0.10 (d, J=1.7 Hz, 6H), 0.85 (d, J=6.3 Hz, 9H), 1.24 (s, 2H), 1.33-1.49 (m, 6H), 1.94-2.02 (m, 2H), 3.07-3.09 (m, 2H), 3.56 (m, 4H), 4.03 (q, J=7.1 Hz, 1H), 4.17 (d, J=3.8 Hz, 1H), 6.27 (dd, J=3.4, 1.7 Hz, 1H), 7.23-7.32 (m, 2H), 7.80 (q, J=1.8, 1.4 Hz, 1H). LCMS m/z Expected 387.2, Observed 388.4.

Cs₂CO₃ (24.13 g, 74.07 mmol) was added to tert-Butyl acrylate (197 mL, 1346.78 mmol) and (3R,4S,5R)—N-(6-((tert-butyldimethylsilyl)oxy)hexyl)-3,4,5-trihydroxycyclohex-1-ene-1-carboxamide (19-2) (8.7 g, 22.45 mmol) in t-BuOH (800 mL). The resulting mixture was stirred at RT for 3 days. The reaction mixture was diluted with EtOAc (1 L), the mixture was filtered through a Celite pad. The solvent was removed under reduced pressure to afford the crude product. The crude product was purified by flash silica chromatography, elution gradient 0 to 50% EtOAc in petroleum ether. Pure fractions were evaporated to dryness to afford tri-tert-butyl 3,3′,3″-(((1R,2S,3R)-5-((6-((tert-butyldimethylsilyl)oxy)hexyl)carbamoyl)cyclohex-4-ene-1,2,3-triyl)tris(oxy))tripropionate (19-3) (8.30 g, 48%) as a yellow oil. The crude product was used in the next step directly without further purification.

1H NMR (400 MHz, DMSO, 24° C.) δ 0.86 (s, 9H), 1.12 (s, 2H), 1.40 (dd, J=4.3, 1.3 Hz, 31H), 1.47-1.58 (m, 2H), 2.36-2.43 (m, 8H), 3.06 (d, J=6.6 Hz, 2H), 3.49 (t, J=6.2 Hz, 1H), 3.65-3.77 (m, 9H), 3.92 (dt, J=21.1, 4.6 Hz, 1H), 6.26-6.31 (m, 1H), 7.87 (tdd, J=8.2, 5.6, 2.8 Hz, 1H). Three protons were exchanged. LCMS m/z Expected 771.5, Observed 772.4.

TBAF (1M in THF) (9.45 mL, 9.45 mmol) was added to tri-tert-butyl 3,3′,3″-(((1R,2S,3R)-5-((6-((tert-butyldimethylsilyl)oxy)hexyl)carbamoyl)cyclohex-4-ene-1,2,3-triyl)tris(oxy))tripropionate (19-3) (7.3 g, 9.45 mmol) in THE (80 mL). The resulting mixture was stirred at RT for 16 hours. The solvent was removed under reduced pressure to afford the crude product. The crude product was purified by flash silica chromatography, elution gradient 0 to 80% EtOAc in petroleum ether. Pure fractions were evaporated to dryness to afford tri-tert-butyl 3,3′,3″-(((1R,2S,3R)-5-((6-hydroxyhexyl)carbamoyl)cyclohex-4-ene-1,2,3-triyl) tris(oxy))tripropionate (19-4) (1.500 g, 24%) as a colorless oil.

1H NMR (400 MHz, DMSO, 23° C.) δ 1.18-1.31 (m, 4H), 1.37-1.45 (m, 29H), 2.16 (dd, J=18.2, 3.6 Hz, 1H), 2.32-2.47 (m, 7H), 3.06 (q, J=6.6 Hz, 2H), 3.17 (d, J=5.2 Hz, 3H), 3.37 (m, 2H), 3.73 (m, 6H), 4.10 (q, J=5.2 Hz, 1H), 4.33 (t, J=5.1 Hz, 1H), 6.29 (d, J=2.5 Hz, 1H), 7.87 (q, J=5.8, 4.5 Hz, 1H). One proton was exchanged. LCMS m/z Expected 657.4, Observed 658.5.

Tri-tert-butyl 3,3′,3″-(((1R,2S,3R)-5-((6-hydroxyhexyl)carbamoyl)cyclohex-4-ene-1,2,3-triyl)tris(oxy))tripropionate (19-4) (1.5 g, 2.28 mmol) and Pd—C(10%) (0.485 g, 0.46 mmol) in MeOH (30 mL) was stirred under an atmosphere of hydrogen at 1 atm and rt for 16 hours. The reaction mixture was filtered through celite. The solvent was removed under reduced pressure to afford crude product. The crude product was purified by flash silica chromatography, elution gradient 10 to 70% EtOAc in petroleum ether. Pure fractions were evaporated to dryness to afford tri-tert-butyl 3,3′,3″-(((1R,2S,3R,5S)-5-((6-hydroxyhexyl)carbamoyl)cyclohexane-1,2,3-triyl)tris(oxy))tripropionate (19-5) (1.000 g, 66%) as a colorless oil.

1H NMR (400 MHz, DMSO, 21° C.) δ 1.05-1.3 (m, 5H), 1.35-1.45 (m, 30H), 1.47-1.67 (m, 4H), 2.18-2.47 (m, 7H), 2.99 (q, J=6.5 Hz, 2H), 3.35-3.45 (m, 3H), 3.47-3.72 (m, 7H), 3.78 (m, 1H), 4.33 (t, J=5.2 Hz, 1H), 7.69 (q, J=6.5, 5.6 Hz, 1H). LCMS m/z Expected 659.4, Observed 660.2.

TFA (3.15 mL, 40.92 mmol) was added to tri-tert-butyl 3,3′,3″-(((1R,2S,3R,5S)-5-((6-hydroxyhexyl)carbamoyl)cyclohexane-1,2,3-triyl)tris(oxy))tripropionate (19-5) (0.9 g, 1.36 mmol) in DCM (10 mL) at rt. The resulting mixture was stirred at rt for 3 hours. The solvent was removed under reduced pressure to afford 3,3′,3″-(((1R,2S,3R,5S)-5-((6-(2,2,2-trifluoroacetoxy)hexyl)carbamoyl)cyclohexane-1,2,3-triyl)tris(oxy))tripropionic acid (19-6) (0.790 g, 99%) as a yellow oil. The product was used in the next step without further purification.

1H NMR (300 MHz, DMSO, 23° C.) δ 1.26-1.4 (m, 6H), 1.46-1.63 (m, 4H), 1.64-1.72 (m, 2H), 2.29 (p, J=6.1 Hz, 1H), 2.35-2.49 (m, 7H), 2.99 (q, J=6.4 Hz, 2H), 3.41 (d, J=8.3 Hz, 1H), 3.59-3.67 (m, 5H), 3.72-3.83 (m, 2H), 4.37 (t, J=6.6 Hz, 2H), 7.70 (t, J=5.5 Hz, 1H), 12.61 (s, 3H). LCMS m/z Expected 587.2, Observed 588.2.

Perfluorophenyl 2,2,2-trifluoroacetate (0.921 mL, 5.36 mmol) was added to 3,3′,3″-(((1R,2S,3R,5S)-5-((6-(2,2,2-trifluoroacetoxy)hexyl)carbamoyl)cyclohexane-1,2,3-triyl)tris(oxy))tripropionic acid (19-6) (0.7 g, 1.19 mmol) and DIEA (1.353 mL, 7.74 mmol) in DCM (20 mL) at rt. The resulting mixture was stirred at rt for 2 hours. The reaction mixture was diluted with DCM (20 ml) and washed sequentially with 1 M NaHSO₄ (2×25 ml), saturated NaHCO₃ (2×15 ml), and saturated brine (30 ml). The organic layer was dried over Na2SO4, filtered and evaporated to afford tris(perfluorophenyl) 3,3′,3″-(((1R,2S,3R,5S)-5-((6-(2,2,2-trifluoroacetoxy)hexyl)carbamoyl)cyclohexane-1,2,3-triyl)tris(oxy))tripropionate (19-7) (1.2 g, 93%) as a yellow oil. The product was used in the next step directly without further purification.

1H NMR (300 MHz, DMSO, 23° C.) δ 1.27-1.43 (m, 6H), 1.53-1.72 (m, 6H), 2.32 (d, J=10.1 Hz, 1H), 2.87-3.11 (m, 11H), 3.74-3.92 (m, 5H), 3.98 (m, 1H), 4.36 (t, J=6.6 Hz, 2H), 7.71 (t, J=5.6 Hz, 1H). LCMS m/z Expected 1085.1, Observed 1086.0.

DIEA (1.448 mL, 8.29 mmol) was added to tris(perfluorophenyl) 3,3′,3″-(((1R,2S,3R,5S)-5-((6-(2,2,2-trifluoroacetoxy)hexyl)carbamoyl)cyclohexane-1,2,3-triyl)tris(oxy))tripropionate (19-7) (1.2 g, 1.11 mmol) and tert-butyl (3-aminopropyl)carbamate (0.867 g, 4.97 mmol) in DCM (30 mL) at rt. The resulting mixture was stirred at rt for 20 hours. The reaction mixture was diluted with DCM (100 ml). The mixture was washed sequentially with saturated brine (2×100 ml). The organic layer was dried over Na₂SO₄, filtered, and evaporated to afford crude product. The crude product was purified by flash silica chromatography, elution gradient 0 to 30% MeOH in DCM. Pure fractions were evaporated to dryness to afford tert-butyl (3-(3-(((1S,2R,4S,6R)-2,6-bis(3-((3-((tert-butoxycarbonyl)amino)propyl)amino)-3-oxopropoxy)-4-((6-hydroxyhexyl)carbamoyl)cyclohexyl)oxy)propanamido)propyl)carbamate (19-8) (0.8 g, 75%) as a yellow oil.

1H NMR (300 MHz, DMSO, 23° C.) δ 1.24 (d, J=6.6 Hz, 9H), 1.38 (d, J=1.4 Hz, 33H), 1.44-1.71 (m, 10H), 2.13-2.42 (m, 6H), 3.00 (m, 10H), 3.12-3.24 (m, 2H), 3.37 (t, J=6.4 Hz, 2H), 3.51-3.74 (m, 6H), 6.66-7.02 (m, 3H), 7.66 (t, J=5.6 Hz, 1H), 7.82 (m, 3H). LCMS m/z Expected 959.6, Observed 960.5.

TFA (3.37 mL, 43.74 mmol) was added to tert-butyl (3-(3-(((1S,2R,4S,6R)-2,6-bis(3-((3-((tert-butoxycarbonyl)amino)propyl)amino)-3-oxopropoxy)-4-((6-hydroxyhexyl)carbamoyl) cyclohexyl)oxy)propanamido)propyl)carbamate (19-8) (700 mg, 0.73 mmol) in DCM (15 mL) at rt. The resulting mixture was stirred at rt for 2 hours. The solvent was removed under reduced pressure to afford 6-((1S,3R,4S,5R)-3,4,5-tris(3-((3-aminopropyl)amino)-3-oxopropoxy) cyclohexane-1-carboxamido)hexyl 2,2,2-trifluoroacetate (19-9) (700 mg, 67%) as a yellow oil. The product was used in the next step without further purification.

1H NMR (300 MHz, DMSO, 23° C.) δ 1.56 (d, J=9.9 Hz, 4H), 1.70 (d, J=9.9 Hz, 9H), 2.33 (q, J=6.4 Hz, 8H), 2.79 (q, J=6.7, 6.1 Hz, 11H), 3.00 (q, J=6.3 Hz, 3H), 3.19-3.41 (m, 3H), 3.49-3.83 (m, 10H), 4.37 (t, J=6.6 Hz, 2H), 7.81 (s, 12H), 8.07 (m, 4H). LCMS m/z Expected 755.4, Observed 756.3.

Perfluorophenyl 2,2,2-trifluoroacetate (2.304 mL, 13.41 mmol) was added to 5-(((2R,3R,4R,5R,6R)-3-acetamido-4,5-diacetoxy-6-(acetoxymethyl)tetrahydro-2H-pyran-2-yl)oxy)pentanoic acid (19-11) (4 g, 8.94 mmol) and DIEA (3.12 mL, 17.88 mmol) in DCM (60 mL) at rt. The resulting mixture was stirred at rt for 2 hours. The reaction mixture was diluted with EtOAc (200 ml), and washed sequentially with 1 M NaHSO₄ (2×250 ml), saturated NaHCO₃ (2×150 ml), and saturated brine (100 ml). The organic layer was dried over Na2SO4, filtered and evaporated to afford (2R,3R,4R,5R,6R)-5-acetamido-2-(acetoxymethyl)-6-((5-oxo-5-(perfluorophenoxy)pentyl)oxy)tetrahydro-2H-pyran-3,4-diyl diacetate (19-12) (5.0 g, 91%) as a yellow oil. The product was used in the next step without further purification.

1H NMR (300 MHz, DMSO, 21° C.) δ 1.45-1.74 (m, 4H), 1.77 (d, J=2.2 Hz, 3H), 1.90 (s, 3H), 2.00 (s, 3H), 2.11 (s, 3H), 2.80 (t, J=7.3 Hz, 2H), 3.48 (dt, J=9.9, 6.1 Hz, 1H), 3.77 (dt, J=10.9, 5.6 Hz, 1H), 3.91 (dt, J=11.2, 8.8 Hz, 1H), 3.98-4.12 (m, 3H), 4.51 (d, J=8.5 Hz, 1H), 4.97 (dd, J=11.3, 3.4 Hz, 1H), 5.23 (d, J=3.4 Hz, 1H), 7.85 (d, J=9.2 Hz, 1H). LCMS m/z Expected 613.1, Observed 614.3.

DIEA (1.941 mL, 11.11 mmol) was added to 6-((1S,3R,4S,5R)-3,4,5-tris(3-((3-aminopropyl)amino)-3-oxopropoxy)cyclohexane-1-carboxamido)hexyl 2,2,2-trifluoroacetate (19-9) (0.7 g, 0.93 mmol) and (2R,3R,4R,5R,6R)-5-acetamido-2-(acetoxymethyl)-6-((5-oxo-5-(perfluorophenoxy)pentyl)oxy)tetrahydro-2H-pyran-3,4-diyl diacetate (19-12) (3.41 g, 5.56 mmol) in DMF (14 mL) at rt. The resulting mixture was stirred at rt for 2 hours. The solvent was removed under reduced pressure to afford the crude product. The crude product was purified by flash C18-flash chromatography, elution gradient 0 to 50% MeCN in water (0.1% FA). Pure fractions were evaporated to dryness to afford [(2R,3R,4R,5R,6R)-5-acetamido-3,4-diacetoxy-6-[5-[3-[3-[(1R,3R)-2,3-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-5-(6-hydroxyhexylcarbamoyl)cyclohexoxy]propanoylamino]propylamino]-5-oxo-pentoxy]tetrahydropyran-2-yl]methyl acetate (19-10) (1 g, 55%) as a yellow solid.

1H NMR (300 MHz, DMSO, 23° C.) δ 1.2-1.63 (m, 33H), 1.78 (s, 9H), 1.90 (s, 9H), 2.03 (d, J=14.4 Hz, 16H), 2.11 (s, 9H), 2.29 (d, J=7.3 Hz, 7H), 2.94-3.12 (m, 14H), 3.14-3.55 (m, 12H), 3.66 (m, 10H), 3.88 (q, J=9.5 Hz, 4H), 4.49 (d, J=8.5 Hz, 3H), 4.97 (dd, J=11.2, 3.4 Hz, 3H), 5.22 (d, J=3.4 Hz, 3H), 7.58-7.99 (m, 10H). LCMS m/z Expected 1946.9, Observed 1948.5.

Chromosulfuric acid (0.436 mL, 0.87 mmol) was added to [(2R,3R,4R,5R,6R)-5-acetamido-3,4-diacetoxy-6-[5-[3-[3-[(1R,3R)-2,3-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-5-(6-hydroxyhexylcarbamoyl)cyclohexoxy]propanoylamino]propylamino]-5-oxo-pentoxy]tetrahydropyran-2-yl]methyl acetate (19-10) (850 mg, 0.44 mmol) in acetone (20 mL) at rt. The resulting mixture was stirred at rt for 2 hours. The reaction mixture was quenched with i-PrOH (20 ml), diluted with DCM (50 ml), and washed sequentially with saturated NaHCO₃ (2×100 ml) and saturated brine (2×100 ml). The organic layer was dried over Na2SO4, filtered, and evaporated to afford crude product. The crude product was purified by flash C18-flash chromatography, elution gradient 0 to 30% MeCN in water (0.1% FA). Pure fractions were evaporated to dryness to afford 6-((3R,5R)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)cyclohexane-1-carboxamido)hexanoic acid (19-13) (458 mg, 53%) as a grey solid.

1H NMR (300 MHz, DMSO, 24° C.) δ 1.25 (s, 3H), 1.35-1.64 (m, 24H), 1.78 (s, 9H), 1.90 (s, 10H), 1.95-2.19 (m, 24H), 2.23 (d, J=29.2 Hz, 9H), 2.81-3.25 (m, 15H), 3.41 (d, J=10.6 Hz, 3H), 3.59-3.78 (m, 11H), 3.89 (t, J=9.8 Hz, 3H), 4.03 (s, 9H), 4.49 (d, J=8.3 Hz, 3H), 4.97 (d, J=11.2 Hz, 3H), 5.22 (s, 3H), 7.64-8.1 (m, 9H), 11.90 (brs, 1H). One proton was exchanged. LCMS m/z Expected 1960.9, Observed 1962.8.

Perfluorophenyl 2,2,2-trifluoroacetate (0.123 mL, 0.71 mmol) was added to (19-13) (200 mg, 0.1 mmol) and DIEA (1.02 mL, 0.74 mmol) in DCM (2 mL) at RT. The resulting mixture was stirred at RT for 4 hours. The reaction mixture was diluted with EtOAc (50 mL), washed sequentially with 1M NaHSO₄ (4×20 mL), saturated NaHCO₃ (2×20 mL), and saturated brine (20 mL). The organic layer was dried over Na₂SO₄, filtered and evaporated to dryness. The residue was diluted with MeCN/water (1:1) 5 mL and dried by lyophilization to give (19) (120 mg, 55%) as off white solid. The product was used in the next reaction without any further purification. LCMS m/z Expected 2126.9, Observed 1065 [M+H]+(z=2).

### Synthesis of Precursor (20)

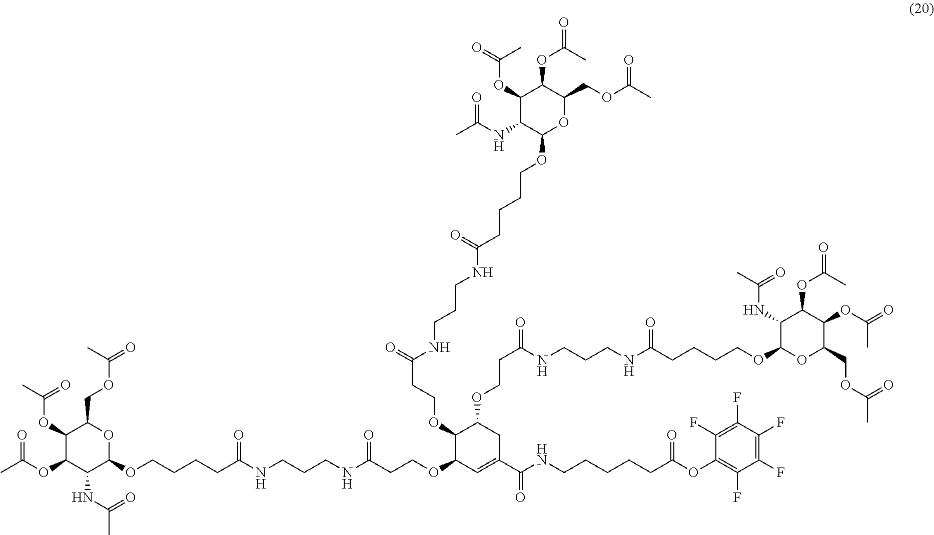 [SMILES: CC(=O)N[C@H]1[C@H](OCCCCC(=O)NCCCNC(=O)CCO[C@@H]2[C@H](OCCC(=O)NCCCNC(=O)CCCCO[C@@H]3O[C@H](COC(C)=O)[C@H](OC(C)=O)[C@H](OC(C)=O)[C@H]3NC(C)=O)C=C(C(=O)NCCCCCC(=O)Oc3c(F)c(F)c(F)c(F)c3F)C[C@H]2OCCC(=O)NCCCNC(=O)CCCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

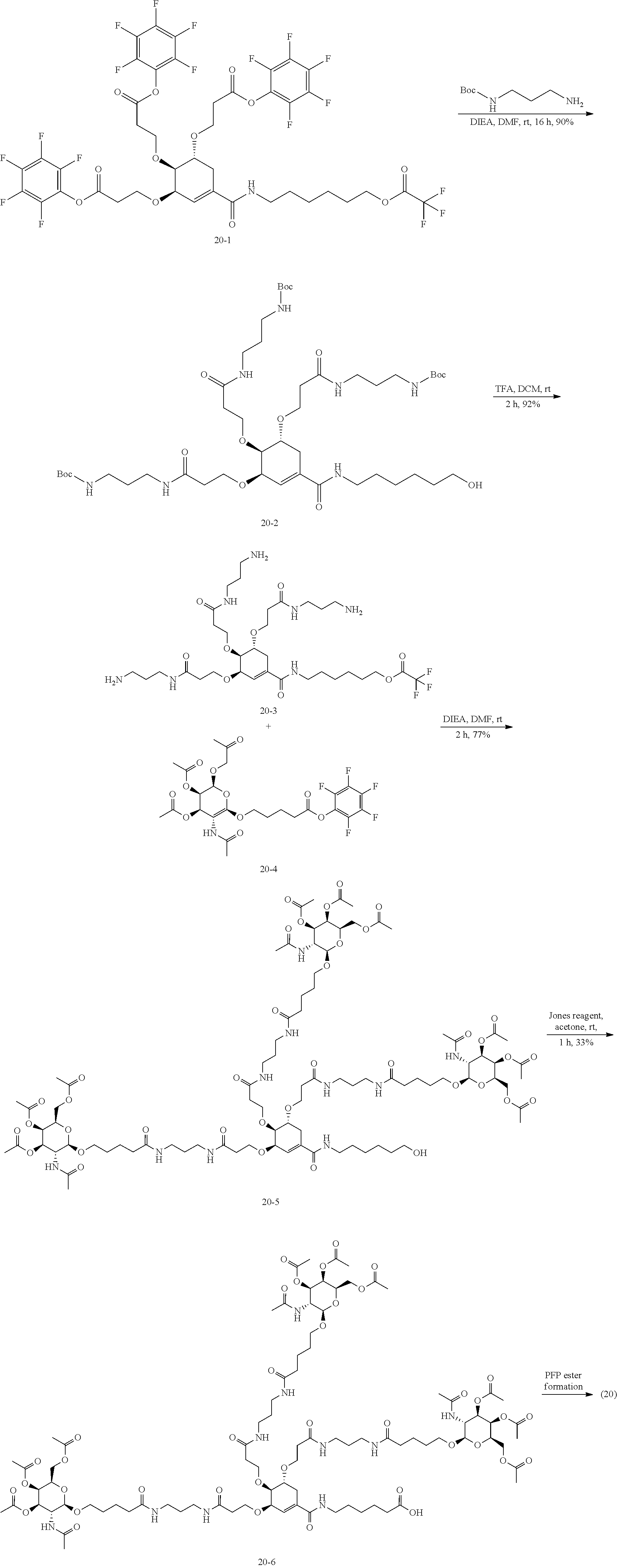

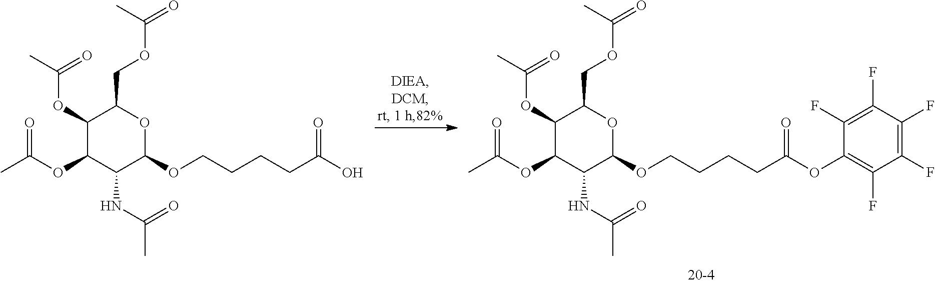 [SMILES: CC(=O)N[C@H]1[C@H](OCCCCC(=O)O)O[C@H](COC(C)=O)[C@H](OC(C)=O)[C@@H]1OC(C)=O ; CC(=O)N[C@H]1[C@H](OCCCCC(=O)Oc2c(F)c(F)c(F)c(F)c2F)O[C@H](COC(C)=O)[C@H](OC(C)=O)[C@@H]1OC(C)=O]

DIEA (3.02 mL, 17.30 mmol) was added to tris(perfluorophenyl) 3,3′,3″-(((1R,2S,3R)-5-((6-(2,2,2-trifluoroacetoxy)hexyl)carbamoyl)cyclohex-4-ene-1,2,3-triyl)tris(oxy))tripropionate (20-1) (2.5 g, 2.31 mmol) and tert-butyl (3-aminopropyl)carbamate (1.8 g, 10.4 mmol) in DMF (30 mL) at RT. The resulting mixture was stirred at RT for 16 hours. The solvent was removed under reduced pressure to afford crude product. The crude product was purified by flash C18-flash chromatography, elution gradient 0 to 80% MeCN in water (0.1% NH₄HCO₃). Pure fractions were evaporated to dryness to afford tert-butyl (3-(3-(((1S,2R,6R)-2,6-bis(3-((3-((tert-butoxycarbonyl)amino)propyl)amino)-3-oxopropoxy)-4-((6-hydroxyhexyl)carbamoyl)cyclohex-3-en-1-yl)oxy)propanamido)propyl)carbamate (20-2) (2.000 g, 90%) as a yellow solid.

1H-NMR (400 MHz, DMSO, 21° C.) δ 1.18-1.29 (m, 5H), 1.37 (s, 27H), 1.42-1.54 (m, 7H), 2.11 (d, J=17.0 Hz, 1H), 2.2-2.3 (m, 4H), 2.85-2.97 (m, 7H), 2.96-3.11 (m, 8H), 3.12-3.21 (m, 1H), 3.33-3.41 (m, 6H), 3.64-3.78 (m, 7H), 4.01 (s, 1H), 4.33 (t, J=5.2 Hz, 1H), 6.26 (s, 1H), 6.75 (t, J=5.7 Hz, 3H), 7.72-7.87 (m, 4H). LCMS m/z Expected 957.6, Observed 958.6.

TFA (20 mL) was added to tert-butyl (3-(3-(((1S,2R,6R)-2,6-bis(3-((3-((tert-butoxycarbonyl)amino)propyl)amino)-3-oxopropoxy)-4-((6-hydroxyhexyl)carbamoyl) cyclohex-3-en-1-yl)oxy)propanamido)propyl)carbamate (20-2) (1.9 g, 1.98 mmol) in DCM (20 mL). The resulting mixture was stirred at RT for 2 hours. The solvent was removed under reduced pressure to afford 6-((3R,4S,5R)-3,4,5-tris(3-((3-aminopropyl)amino)-3-oxopropoxy)cyclohex-1-ene-1-carboxamido)hexyl 2,2,2-trifluoroacetate (20-3) (2.000 g, 92%) as a yellow oil. The product was used in the next step directly without further purification.

1H-NMR (400 MHz, DMSO, 21° C.) δ 1.24-1.35 (m, 5H), 1.37-1.46 (m, 2H), 1.56-1.74 (m, 8H), 2.22-2.44 (m, 7H), 2.69-2.88 (m, 8H), 3.04-3.17 (m, 8H), 3.23-3.28 (m, 1H), 3.6-3.79 (m, 8H), 4.01 (s, 1H), 4.37 (t, J=6.6 Hz, 2H), 6.28 (s, 1H), 7.93 (t, J=5.7 Hz, 1H), 7.99-8.15 (m, 3H). LCMS m/z Expected 753.4, Observed 754.4.

Perfluorophenyl 2,2,2-trifluoroacetate (7.51 g, 26.82 mmol) was added to 5-(((2R,3R,4R,5R,6R)-3-acetamido-4,5-diacetoxy-6-(acetoxymethyl)tetrahydro-2H-pyran-2-yl)oxy)pentanoic acid (8 g, 17.88 mmol) and DIEA (6.25 mL, 35.76 mmol) in DCM (100 mL) at 0° C. under nitrogen. The resulting mixture was stirred at RT for 2 hours. The reaction mixture was diluted with DCM (350 ml), and washed sequentially with 1 M NaHSO₄ (2×300 ml), saturated NaHCO₃ (2×300 ml), and saturated brine (300 ml). The organic layer was dried over Na2SO4, filtered and evaporated to afford (2R,3R,4R,5R,6R)-5-acetamido-2-(acetoxymethyl)-6-((5-oxo-5-(perfluorophenoxy)pentyl)oxy)tetrahydro-2H-pyran-3,4-diyl diacetate (20-4) (9.00 g, 82%) as a yellow oil. The product was used in the next step directly without further purification.

1H NMR (400 MHz, DMSO, 21° C.) δ 1.57-1.61 (m, 2H), 1.63-1.72 (m, 2H), 1.76 (s, 3H), 1.89 (s, 3H), 1.99 (s, 3H), 2.10 (s, 3H), 2.79 (t, J=7.4 Hz, 2H), 3.43-3.51 (m, 1H), 3.71-3.8 (m, 1H), 3.84-3.95 (m, 1H), 4.03 (d, J=17.0 Hz, 3H), 4.50 (d, J=8.5 Hz, 1H), 4.96 (dd, J=11.3, 3.4 Hz, 1H), 5.22 (d, J=3.4 Hz, 1H), 7.85 (d, J=9.3 Hz, 1H). LCMS m/z Expected 613.2, Observed 614.1.

DIEA (1.968 mL, 11.27 mmol) was added to 6-((3R,4S,5R)-3,4,5-tris(3-((3-aminopropyl)amino)-3-oxopropoxy)cyclohex-1-ene-1-carboxamido)hexyl 2,2,2-trifluoroacetate (20-3) (1.9 g, 1.73 mmol) and (2R,3R,4R,5R,6R)-5-acetamido-2-(acetoxymethyl)-6-((5-oxo-5-(perfluorophenoxy)pentyl)oxy)tetrahydro-2H-pyran-3,4-diyl diacetate (20-4) (4.79 g, 7.80 mmol) in DMF (30 mL) at RT. The resulting mixture was stirred at RT for 2 hours. The solvent was removed under reduced pressure to afford crude product. The crude product was purified by flash C18-flash chromatography, elution gradient 0 to 50% MeCN in water (0.1% TFA). Pure fractions were evaporated to dryness to afford [(2R,3R,4R,5R,6R)-5-acetamido-3,4-diacetoxy-6-[5-[3-[3-[(1R,5R,6S)-5,6-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-3-(6-hydroxyhexylcarbamoyl)cyclohex-3-en-1-yl]oxypropanoylamino]propylamino]-5-oxo-pentoxy]tetrahydropyran-2-yl]methyl acetate (20-5) (2.60 g, 77%) as a yellow solid.

1H-NMR (500 MHz, DMSO, 25° C.) δ 1.17-1.29 (m, 4H), 1.36-1.53 (m, 22H), 1.77 (s, 9H), 1.89 (s, 9H), 2.00 (s, 9H), 2.04 (t, J=7.2 Hz, 7H), 2.10 (s, 9H), 2.21-2.41 (m, 7H), 2.99-3.07 (m, 12H), 3.34-3.43 (m, 5H), 3.61-3.77 (m, 14H), 3.83-3.9 (m, 4H), 3.99-4.06 (m, 10H), 4.48 (d, J=8.4 Hz, 3H), 4.96 (dd, J=11.3, 3.4 Hz, 3H), 5.21 (d, J=3.4 Hz, 3H), 6.27 (s, 1H), 7.7-7.76 (m, 3H), 7.77-7.86 (m, 6H). LCMS m/z Expected 1944.9, Observed 973.9 (M+1, z=2).

Chromosulfuric acid (0.771 mL, 1.54 mmol) was added to [(2R,3R,4R,5R,6R)-5-acetamido-3,4-diacetoxy-6-[5-[3-[3-[(1R,5R,6S)-5,6-bis[3-[3-[5-[(2R,3R,4R,5R,6R)-3-acetamido-4,5-diacetoxy-6-(acetoxymethyl)tetrahydropyran-2-yl]oxypentanoylamino]propylamino]-3-oxo-propoxy]-3-(6-hydroxyhexylcarbamoyl)cyclohex-3-en-1-yl]oxypropanoylamino]propylamino]-5-oxo-pentoxy]tetrahydropyran-2-yl]methyl acetate (20-5) (1.5 g, 0.77 mmol) in acetone (20 mL) at 0° C. The resulting mixture was stirred at RT for 1 hour. The reaction mixture was quenched with i-PrOH (10 ml), the mixture was diluted with DCM (10 ml), after 10 min, saturated NaHCO₃ (20 ml) was added, at last, the solution was evaporated to afford crude product. The crude product was purified by flash C18-flash chromatography, elution gradient 0 to 40% MeCN in water (0.1% TFA). Pure fractions were evaporated to dryness to afford 6-((3R,4S,5R)-3,4,5-tris(3-((3-(5-(((2R,3R,4R,5R,6R)-3-acetamido-4,5-diacetoxy-6-(acetoxymethyl)tetrahydro-2H-pyran-2-yl)oxy)pentanamido)propyl)amino)-3-oxopropoxy)cyclohex-1-ene-1-carboxamido)hexanoic acid (20-6) (0.504 g, 33%) as a white solid.

1H-NMR (500 MHz, DMSO, 25° C.) δ 1.18-1.29 (m, 2H), 1.34-1.58 (m, 23H), 1.77 (s, 9H), 1.88 (s, 9H), 1.9-1.94 (m, 2H), 2.00 (s, 9H), 2.02-2.07 (m, 7H), 2.10 (s, 9H), 2.21-2.29 (m, 4H), 2.33 (q, J=6.5, 4.6 Hz, 2H), 2.92-3.1 (m, 14H), 3.40 (dt, J=9.7, 6.2 Hz, 3H), 3.62-3.81 (m, 11H), 3.83-3.95 (m, 3H), 3.96-4.08 (m, 10H), 4.54 (d, J=8.5 Hz, 3H), 4.99 (dd, J=11.2, 3.5 Hz, 3H), 5.21 (d, J=3.4 Hz, 3H), 6.29 (s, 1H), 7.84-7.93 (m, 2H), 7.96-8.05 (m, 4H), 8.12 (s, 4H). LCMS m/z Expected 1958.9, Observed 980.9 (M+1, z=2).

Precursor (20) is prepared analogously to the PFP esters described above. For example, perfluorophenyl 2,2,2-trifluoroacetate is added to (20-6) and DIEA in DCM at RT. The resulting mixture is stirred at RT. The reaction mixture is diluted with EtOAc, washed with aqueous salt solutions (e.g., with 1M NaHSO₄, saturated NaHCO₃, and saturated brine), and the organic layer dried over Na₂SO₄, filtered and evaporated to dryness. The residue is diluted with solvent, e.g. MeCN/water (1:1), and dried by lyophilization to give (20). The product is used in the next reaction without any further purification.

### Synthesis of Precursor (21)

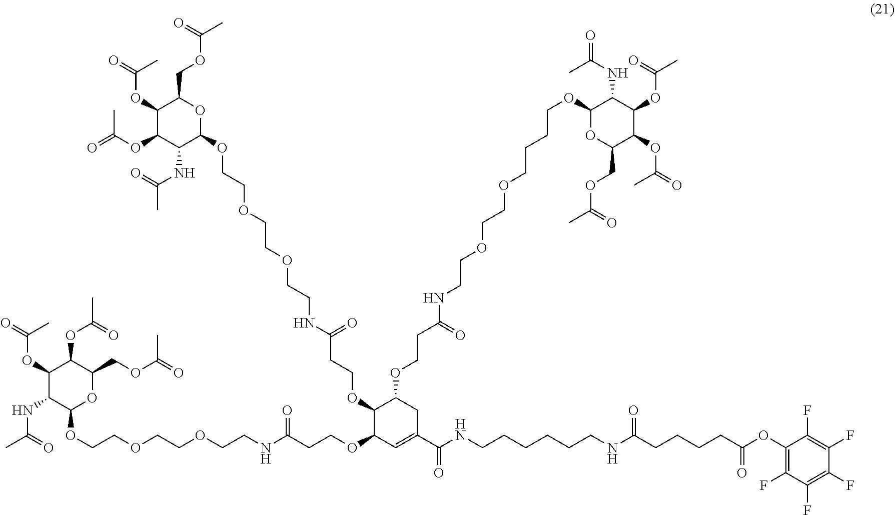 [SMILES: CC(=O)N[C@H]1[C@H](OCCCCOCCOCCNC(=O)CCO[C@@H]2CC(C(=O)NCCCCCCNC(=O)CCCCC(=O)Oc3c(F)c(F)c(F)c(F)c3F)=C[C@@H](OCCC(=O)NCCOCCOCCO[C@@H]3O[C@H](COC(C)=O)[C@H](OC(C)=O)[C@H](OC(C)=O)[C@H]3NC(C)=O)[C@H]2OCCC(=O)NCCOCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Synthetic scheme:

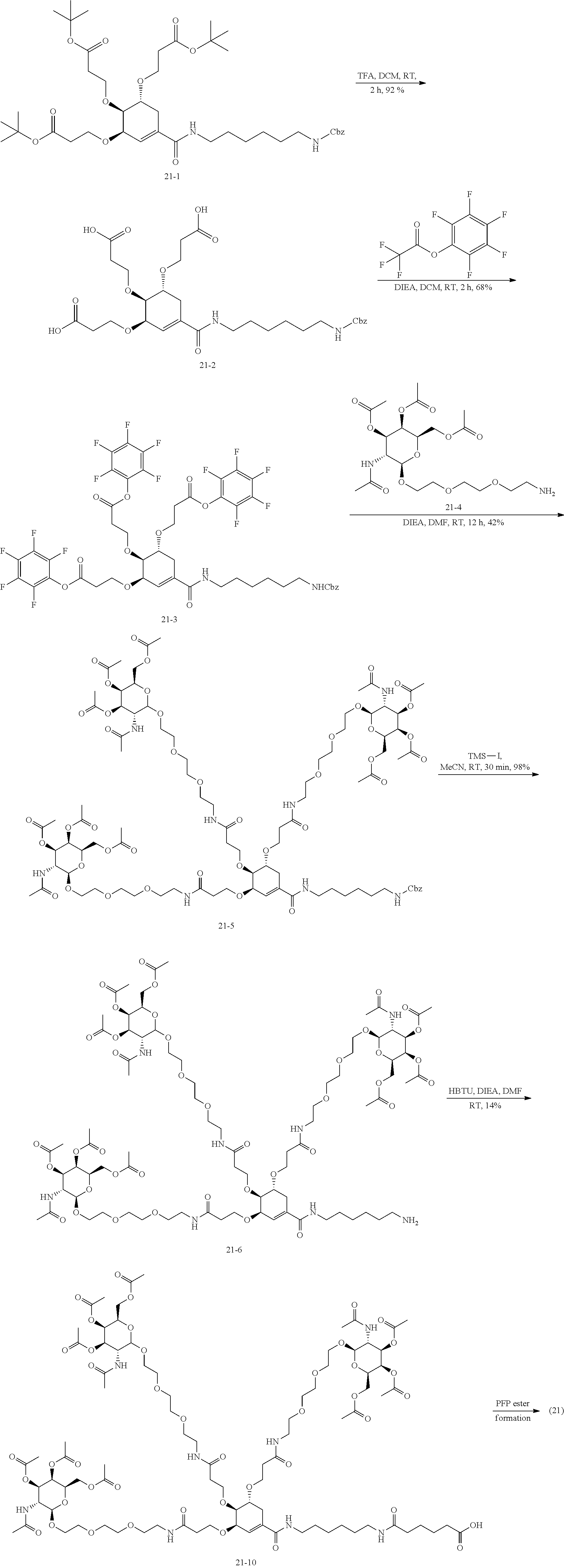

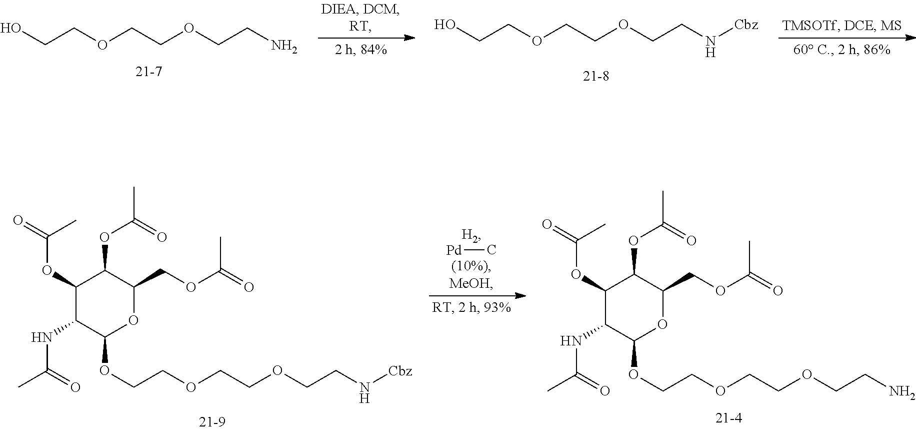

Tri-tert-butyl 3,3′,3″-(((1R,2S,3R)-5-((6-(((benzyloxy)carbonyl)amino)hexyl) cyclohex-4-ene-1,2,3-triyl)tris(oxy))tripropionate (21-1) (1.8 g, 2.28 mmol) was added in TFA (10 mL) and DCM (10.00 mL). The resulting mixture was stirred at RT for 2 hours. The reaction mixture was evaporated to afford 3,3′,3″-(((1R,2S,3R)-5-((6-(((benzyloxy)carbonyl)amino)hexyl) carbamoyl)cyclohex-4-ene-1,2,3-triyl)tris(oxy))tripropionic acid (21-2) (1.300 g, 92%) as a yellow oil. The product was used in the next step directly without further purification.

1H-NMR (500 MHz, DMSO, 25° C.) δ 1.24 (s, 5H), 1.36-1.42 (m, 5H), 2.4-2.47 (mn, 6H), 2.97 (d, J=6.3 Hz, 2H), 3.04-3.08 (mn, 2H), 3.21 (q, J=8.5, 7.5 Hz, 1H), 3.66-3.68 (mn, 1H), 3.69-3.84 (m, 6H), 4.06-4.08 (m, 1H), 5.00 (s, 2H), 6.26 (d, J=3.2 Hz, 1H), 7.18-7.25 (m, 1H), 7.31-7.37 (m, 5H), 7.81-7.92 (m, 1H). LCMS m/z Expected 622.3, Observed 623.4.

Perfluorophenyl 2,2,2-trifluoroacetate (1.614 mL, 9.40 mmol) was added to 3,3′,3″-(((1R,2S,3R)-5-((6-(((benzyloxy)carbonyl)amino)hexyl)carbamoyl)cyclohex-4-ene-1,2,3-triyl)tris(oxy))tripropionic acid (21-2) (1.3 g, 2.09 mmol) and DIEA (2.55 mL, 14.61 mmol) in DCM (30 mL) at 0° C. The resulting mixture was stirred at RT for 2 hours. The reaction mixture was evaporated to afford tris(perfluorophenyl) 3,3′,3″-(((1R,2S,3R)-5-((6-(((benzyloxy) carbonyl)amino)hexyl)carbamoyl)cyclohex-4-ene-1,2,3-triyl)tris(oxy))tripropionate (21-3) (1.600 g, 68%) as a yellow oil. The product was used in the next step directly without further purification.

1H-NMR (500 MHz, DMSO, 25° C.) δ 1.2-1.28 (m, 10H), 2.16-2.3 (m, 1H), 2.39-2.49 (m, 1H), 2.91-3.12 (m, 8H), 3.18-3.33 (m, 1H), 3.53-3.63 (m, 1H), 3.76-3.98 (m, 6H), 4.16 (s, 1H), 4.99 (s, 2H), 6.35 (d, J=3.0 Hz, 1H), 7.21 (q, J=6.1, 4.6 Hz, 1H), 7.33 (q, J=6.6, 6.2 Hz, 5H), 7.87 (t, J=5.6 Hz, 1H). LCMS m/z Expected 1120.8, Observed 1143.4.

Benzyl (2,5-dioxopyrrolidin-1-yl) carbonate (20.88 g, 83.79 mmol) was added slowly to 2-(2-(2-aminoethoxy)ethoxy)ethan-1-ol (10 g, 67.03 mmol) and DIEA (35.1 mL, 201.09 mmol) in DCM (100 mL). The resulting mixture was stirred at RT for 2 hours. The reaction mixture was evaporated to afford crude product. The crude product was purified by flash silica chromatography, elution gradient 0 to 20% MeOH in DCM. (product runs at 10%). Pure fractions were evaporated to dryness to afford benzyl (2-(2-(2-hydroxyethoxy)ethoxy)ethyl)carbamate (21-8) (16.00 g, 84%) as a colorless liquid.

1H NMR (300 MHz, DMSO, 23° C.) δ 3.16 (q, J=5.9 Hz, 2H), 3.42 (td, J=5.3, 4.4, 2.8 Hz, 4H), 3.50 (d, J=6.0 Hz, 6H), 4.60 (t, J=5.4 Hz, 1H), 5.03 (s, 2H), 7.28-7.31 (m, 1H), 7.32-7.41 (m, 5H). LCMS m/z Expected 283.1, Observed 284.1.

Benzyl (2-(2-(2-hydroxyethoxy)ethoxy)ethyl)carbamate (9.68 g, 34.16 mmol) was added to (5R,6R,7R,7aR)-5-(acetoxymethyl)-2-methyl-3a,6,7,7a-tetrahydro-5H-pyrano[3,2-d]oxazole-6,7-diyl diacetate (15 g, 34.16 mmol) and Molecular sieves (1.5 g, 0.00 mmol) in DCE (100 mL) at RT. TMS-OTf (6.17 mL, 34.16 mmol) was added after the reaction had been stirred for 30 minutes at RT. The resulting mixture was stirred at 60° C. for 2 hours. The reaction mixture was quenched with saturated NaHCO₃ (300 mL), extracted with DCM (3×250 mL). The organic layer was washed with saturated brine (2×300 mL), dried over Na₂SO₄, filtered and evaporated to dryness to afford dark brown oil. The crude product was purified by flash silica chromatography, elution gradient 0 to 100% EtOAc in petroleum ether. Pure fractions were evaporated to dryness to afford (2R,3R,4R,5R,6R)-5-acetamido-2-(acetoxymethyl)-6-((3-oxo-1-phenyl-2,7,10-trioxa-4-azadodecan-12-yl)oxy)tetrahydro-2H-pyran-3,4-diyl diacetate (21-9) (18.00 g, 86%) as a yellow oil.

1H NMR (500 MHz, DMSO, 25° C.) δ 1.77 (s, 3H), 1.89 (s, 3H), 2.00 (s, 3H), 2.10 (s, 3H), 3.15 (d, J=5.9 Hz, 2H), 3.42 (d, J=6.0 Hz, 2H), 3.50 (q, J=4.4, 2.6 Hz, 8H), 3.59 (dt, J=8.5, 4.0 Hz, 1H), 3.78 (ddd, J=11.1, 5.6, 3.8 Hz, 1H), 3.88 (dt, J=11.3, 8.8 Hz, 1H), 4.03 (d, J=2.1 Hz, 2H), 4.55 (d, J=8.5 Hz, 1H), 5.01 (s, 2H), 5.22 (d, J=3.5 Hz, 1H), 7.26-7.34 (m, 1H), 7.31-7.4 (m, 5H), 7.80 (d, J=9.2 Hz, 1H). LCMS m/z Expected 612.3, Observed 613.4.

Pd—C(10%) (1.911 g, 1.80 mmol) was added to (2R,3R,4R,5R,6R)-5-acetamido-2-(acetoxymethyl)-6-((3-oxo-1-phenyl-2,7,10-trioxa-4-azadodecan-12-yl)oxy)tetrahydro-2H-pyran-3,4-diyl diacetate (5.5 g, 8.98 mmol) in MeOH (60 mL) under hydrogen. The resulting mixture was stirred at RT for 2 hours. The reaction mixture was filtered through celite and evaporated to dryness to afford (2R,3R,4R,5R,6R)-5-acetamido-2-(acetoxymethyl)-6-(2-(2-(2-aminoethoxy)ethoxy)ethoxy)tetrahydro-2H-pyran-3,4-diyl diacetate (21-4) (4.00 g, 93%) as a yellow solid. The product was used in the next step directly without further purification.

1H NMR (500 MHz, DMSO, 25° C.) δ 1.78 (d, J=3.7 Hz, 3H), 1.89 (s, 3H), 2.00 (s, 3H), 2.10 (s, 3H), 2.89 (t, J=5.4 Hz, 2H), 3.51-3.61 (m, 10H), 3.73-3.93 (m, 2H), 3.97-4.2 (m, 4H), 4.55 (d, J=8.4 Hz, 1H), 4.96-4.99 (m, 1H), 5.22 (d, J=3.3 Hz, 1H), 7.90 (d, J=9.2 Hz, 1H). LCMS m/z Expected 478.2, Observed 479.3.

DIEA (1.995 mL, 11.42 mmol) was added to tris(perfluorophenyl) 3,3′,3″-(((1R,2S,3R)-5-((6-(((benzyloxy)carbonyl)amino)hexyl)carbamoyl)cyclohex-4-ene-1,2,3-triyl)tris(oxy))tripropionate (21-3) (1.6 g, 1.43 mmol) and (2R,3R,4R,5R,6R)-5-acetamido-2-(acetoxymethyl)-6-(2-(2-(2-aminoethoxy)ethoxy)ethoxy)tetrahydro-2H-pyran-3,4-diyl diacetate (21-4) (3.07 g, 6.42 mmol) in DMF (60 mL). The resulting mixture was stirred at RT for 12 hours. The reaction mixture was evaporated to afford crude product. The crude product was purified by flash C18-flash chromatography (220 g), elution gradient 20 to 80% MeCN in water (0.1% FA). Pure fractions were evaporated to dryness to afford [(2R,3R,4R,5R,6R)-5-acetamido-3,4-diacetoxy-6-[2-[2-[2-[3-[(1R,5R,6S)-5,6-bis[3-[2-[2-[2- [(2R,3R,4R,5R,6R)-3-acetamido-4,5-diacetoxy-6-(acetoxymethyl)tetrahydropyran-2-yl]oxyethoxy]ethoxy]ethylamino]-3-oxo-propoxy]-3-[6-(benzyloxycarbonylamino)hexylcarbamoyl]cyclohex-3-en-1-yl]oxypropanoylamino]ethoxy]ethoxy]ethoxy]tetrahydropyran-2-yl]methyl acetate (21-5) (1.200 g, 42%) as a yellow solid.

1H NMR (500 MHz, DMSO, 25° C.) δ 1.2-1.27 (m, 6H), 1.38 (d, J=6.8 Hz, 4H), 1.77 (s, 9H), 1.81 (s, 2H), 1.89 (s, 10H), 2.00 (s, 9H), 2.10 (s, 9H), 2.27-2.39 (m, 6H), 2.97 (q, J=6.6 Hz, 3H), 3.05 (q, J=6.6 Hz, 2H), 3.14-3.24 (m, 8H), 3.35-3.43 (m, 8H), 3.49-3.52 (m, 16H), 3.56-3.61 (m, 4H), 3.63-3.84 (m, 10H), 3.85-3.91 (m, 3H), 4.03 (s, 5H), 4.55 (d, J=8.5 Hz, 3H), 4.96-5 (m, 4H), 5.22 (d, J=3.4 Hz, 3H), 6.26 (s, 1H), 7.22 (t, J=5.6 Hz, 1H), 7.28-7.32 (m, 1H), 7.32-7.37 (m, 5H), 7.77-8.01 (m, 6H). LCMS m/z Expected 2002.9, Observed 1003.0 (z=2).

TMS-I (0.815 mL, 5.99 mmol) was added to [(2R,3R,4R,5R,6R)-5-acetamido-3,4-diacetoxy-6-[2-[2-[2-[3-[(1R,5R,6S)-5,6-bis[3-[2-[2-[2-[(2R,3R,4R,5R,6R)-3-acetamido-4,5-diacetoxy-6-(acetoxymethyl)tetrahydropyran-2-yl]oxyethoxy]ethoxy]ethylamino]-3-oxo-propoxy]-3-[6-(benzyloxycarbonylamino)hexylcarbamoyl]cyclohex-3-en-1-yl]oxypropanoylamino]ethoxy]ethoxy]ethoxy]tetrahydropyran-2-yl]methyl acetate (21-5) (1.2 g, 0.60 mmol) in MeCN (10 mL). The resulting mixture was stirred at RT for 30 minutes. The reaction mixture was evaporated to afford [(2R,3R,4R,5R,6R)-5-acetamido-3,4-diacetoxy-6-[2-[2-[2-[3-[(1R,5R,6S)-5,6-bis[3-[2-[2-[2-[(2R,3R,4R,5R,6R)-3-acetamido-4,5-diacetoxy-6-(acetoxymethyl)tetrahydropyran-2-yl]oxyethoxy]ethoxy]ethylamino]-3-oxo-propoxy]-3-(6-aminohexylcarbamoyl)cyclohex-3-en-1-yl]oxypropanoylamino]ethoxy]ethoxy]ethoxy]tetrahydropyran-2-yl]methyl acetate (21-6) (1.100 g, 98%) as a yellow solid. The product was used in the next step directly without further purification.

1H NMR (500 MHz, DMSO, 25° C.) δ 1.2-1.58 (m, 10H), 1.77 (s, 6H), 1.81 (d, J=2.7 Hz, 3H), 1.89 (s, 9H), 1.99 (d, J=2.8 Hz, 9H), 2.10 (d, J=2.3 Hz, 9H), 2.28 (d, J=6.8 Hz, 4H), 2.33-2.37 (m, 2H), 2.74-2.78 (m, 2H), 3.07 (d, J=6.5 Hz, 2H), 3.15-3.27 (m, 9H), 3.39-3.43 (m, 9H), 3.48-3.53 (m, 21H), 3.56-3.61 (m, 4H), 3.69-3.75 (m, 3H), 3.86-3.9 (m, 2H), 4.03 (d, J=3.1 Hz, 6H), 4.17-4.25 (m, 2H), 4.55 (d, J=8.5 Hz, 2H), 4.90 (d, J=3.6 Hz, 1H), 4.98 (d, J=3.4 Hz, 1H), 5.21 (d, J=3.4 Hz, 2H), 6.26 (s, 1H), 7.31 (d, J=5.4 Hz, 1H), 7.44-7.52 (m, 1H), 7.51-7.65 (m, 5H), 7.81 (d, J=9.2 Hz, 2H), 7.84-7.95 (m, 3H), 7.99 (d, J=8.1 Hz, 1H). LCMS m/z Expected 1868.9, Observed 935.8 (z=2).

DIEA (1.027 mL, 5.88 mmol) was added to [(2R,3R,4R,5R,6R)-5-acetamido-3,4-diacetoxy-6-[2-[2-[2-[3-[(1R,5R,6S)-5,6-bis[3-[2-[2-[2-[(2R,3R,4R,5R,6R)-3-acetamido-4,5-diacetoxy-6-(acetoxymethyl)tetrahydropyran-2-yl]oxyethoxy]ethoxy]ethylamino]-3-oxo-propoxy]-3-(6-aminohexylcarbamoyl)cyclohex-3-en-1-yl]oxypropanoylamino]ethoxy]ethoxy]ethoxy]tetrahydropyran-2-yl]methyl acetate (21-6) (1.1 g, 0.59 mmol), adipic acid (0.430 g, 2.94 mmol) and O-(Benzotriazol-1-yl)-N—N—N′,N′-tetramethyluronium hexafluorophosphate (0.669 g, 1.76 mmol) in DMF (10 mL). The resulting mixture was stirred at RT for 2 hours.

The reaction mixture was evaporated to afford crude product. The crude product was purified by flash C18-flash chromatography, elution gradient 10 to 60% MeCN in water (0.1% TFA) (product runs at 30%). Pure fractions were evaporated to dryness to afford 6-oxo-6-((6-((3R,4S,5R)-3,4,5-tris(3-((2-(2-(2-(((2R,3R,4R,5R,6R)-3-acetamido-4,5-diacetoxy-6-(acetoxymethyl)tetrahydro-2H-pyran-2-yl)oxy)ethoxy)ethoxy)ethyl)amino)-3-oxopropoxy)cyclohex-1-ene-1-carboxamido)hexyl)amino)hexanoic acid (21-10) (0.164 g, 14%) as a pale yellow solid.

1H NMR (500 MHz, DMSO, 25° C.) δ 1.2-1.29 (m, 4H), 1.31-1.55 (m, 10H), 1.79 (d, J=18.1 Hz, 8H), 1.90 (d, J=8.1 Hz, 8H), 1.96-2.05 (m, 11H), 2.06-2.14 (m, 9H), 2.19 (t, J=6.9 Hz, 3H), 2.24-2.41 (m, 8H), 3.02 (dq, J=25.3, 6.6 Hz, 4H), 3.19 (q, J=6.7 Hz, 7H), 3.35-3.44 (m, 7H), 3.47-3.58 (m, 16H), 3.58 (q, J=7.4, 6.2 Hz, 5H), 3.73-3.78 (m, 10H), 3.88 (d, J=9.7 Hz, 3H), 3.96-4.08 (m, 7H), 4.1-4.12 (m, 1H), 4.21-4.25 (m, 1H), 4.55 (d, J=8.5 Hz, 1H), 4.90 (d, J=3.6 Hz, 1H), 4.97-5.03 (m, 3H), 5.21 (d, J=3.5 Hz, 2H), 5.31 (d, J=3.3 Hz, 1H), 6.26 (s, 1H), 7.67-8.02 (m, 8H). LCMS m/z Expected 1996.9, Observed 999.8 (z=2).

Precursor (21) is prepared analogously to the PFP esters described above. For example, perfluorophenyl 2,2,2-trifluoroacetate is added to (21-10) and DIEA in DCM at RT. The resulting mixture is stirred at RT. The reaction mixture is diluted with EtOAc, washed with aqueous salt solutions (e.g., with 1M NaHSO₄, saturated NaHCO₃, and saturated brine), and the organic layer dried over Na₂SO₄, filtered and evaporated to dryness. The residue is diluted with solvent, e.g. MeCN/water (1:1), and dried by lyophilization to give (21). The product is used in the next reaction without any further purification.

### Example 1—Synthesis of Test Compounds

### Compound 1: Pentanoyl Propanediamine/C₁ Spacer, BCN Tether

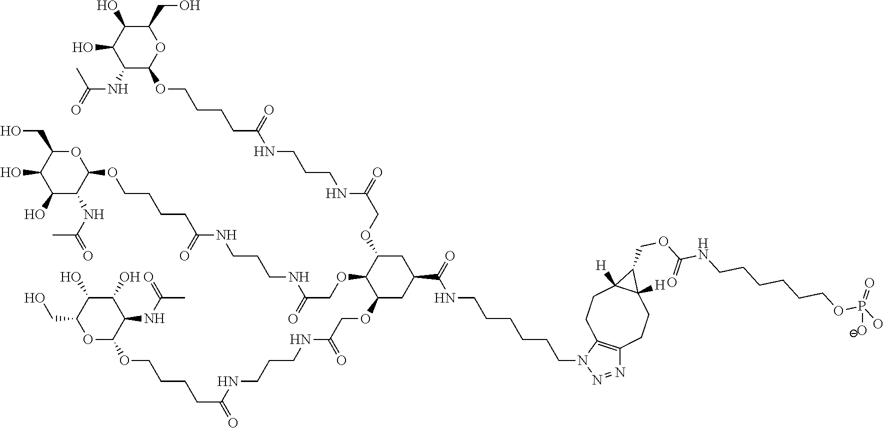 [SMILES: CC(=O)N[C@H]1[C@H](OCCCCC(=O)NCCCNC(=O)CO[C@@H]2C[C@H](C(=O)NCCCCCCn3nnc4c3CC[C@H]3[C@@H](CC4)[C@H]3COC(=O)NCCCCCCOP(=O)([O-])O)C[C@@H](OCC(=O)NCCCNC(=O)CCCCO[C@@H]3O[C@H](CO)[C@H](O)[C@H](O)[C@H]3NC(C)=O)[C@@H]2OCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

BCN modified HA MALAT1 ASO (25 mg, 1 equiv.) was dissolved in deionized water (0.5 mL). Precursor (1) (10.80 mg, 1.5 equiv.) was dissolved in 0.2 mL Acetonitrile. Added acetonitrile solution of precursor (1) to ASO solution under stirring. Clear solution was seen. Reaction mixture was stirred at RT for 20 h. LCMS after 20 h showed main peak of expected product (acetyl protected). The mixture was diluted with 2 mL of 25% aq. NH₃ and left under stirring at RT for 5h when acetyl groups got hydrolysed. Volatiles were removed under vacuum. The crude product was dissolved in Water:Acetonitrile (1:1, 2 mL) and freeze dried. Obtained white solid was purified by Prep HPLC. Pure fractions were freeze dried to obtain white solid (12 mg, 38% Yield). LCMS m/z ES⁻ Expected 6940.289, observed 1736.0813 (z=4), 1389.0670 (z=5).

### Compound 2a: Hexylamine Spacer/Tether

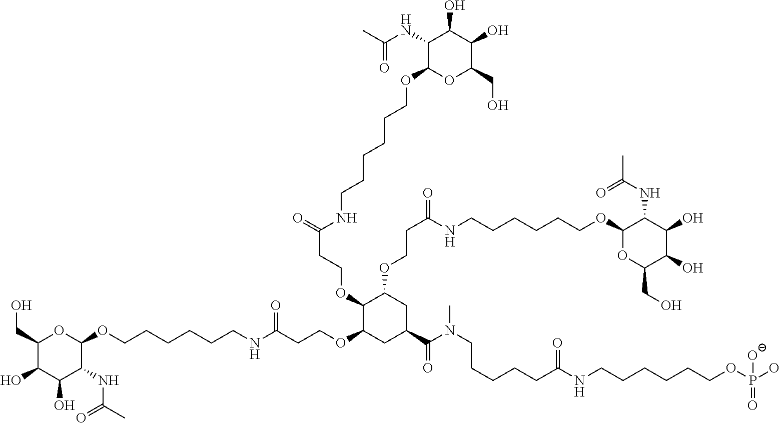 [SMILES: CC(=O)N[C@H]1[C@H](OCCCCCCNC(=O)CCO[C@@H]2C[C@H](C(=O)N(C)CCCCCC(=O)NCCCCCCOP(=O)([O-])O)C[C@@H](OCCC(=O)NCCCCCCO[C@@H]3O[C@H](CO)[C@H](O)[C@H](O)[C@H]3NC(C)=O)[C@@H]2OCCC(=O)NCCCCCCO[C@@H]2O[C@H](CO)[C@H](O)[C@H](O)[C@H]2NC(C)=O)O[C@H](CO)[C@H](O)[C@@H]1O]

Passenger and guide strands were synthesized as described above. Passenger strand was conjugated with GalNAc to form the GalNac-HA-invAb*gcucaacaUAUuugaucagua*invAb (PO to HA) complex.

Passenger strand (PO to HA) (60 mg, 1 equiv.) was dissolved in borate buffer (pH 9, 1 ml). GalNAc PFP ester, Precursor 2 (22 mg, 1.5 equiv.), was dissolved in ACN (0.2 mL). This ACN solution was added to borate buffer solution gradually and the reaction mixture was stirred. After 2 h, full conversion was seen by LCMS. Next, aq. ammonia (1 mL) was added to the reaction mixture and shaken overnight to hydrolyze acetyl groups. The solvent was evaporated and compound was purified by HPLC. Pure fractions were freeze dried. Obtained white solid was dissolved in 0.3 mL water and product was precipitated by using aq. NaOAc and ethanol. Centrifuged product was freeze dried to furnish sodium salt of passenger strand as a white solid. LCMS m/z ES⁻ Expected 8876.002, observed 985.678 (z=9)

Guide strand was synthesized following general synthetic protocol. Passenger strand and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. Product solution (Compound 2a) was adjusted to 1 mM in PBS buffer.

### Compound 2b:

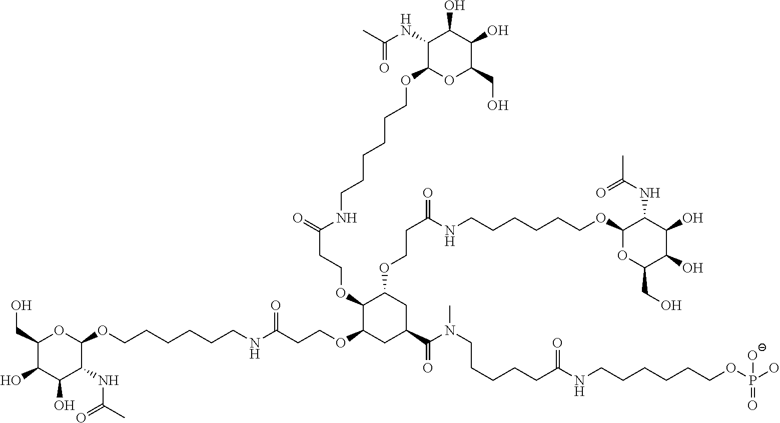 [SMILES: CC(=O)N[C@H]1[C@H](OCCCCCCNC(=O)CCO[C@@H]2C[C@H](C(=O)N(C)CCCCCC(=O)NCCCCCCOP(=O)([O-])O)C[C@@H](OCCC(=O)NCCCCCCO[C@@H]3O[C@H](CO)[C@H](O)[C@H](O)[C@H]3NC(C)=O)[C@@H]2OCCC(=O)NCCCCCCO[C@@H]2O[C@H](CO)[C@H](O)[C@H](O)[C@H]2NC(C)=O)O[C@H](CO)[C@H](O)[C@@H]1O]

Passenger strand (20-mer, SEQ ID NO: 5) and a guide strand (SEQ ID NO: 6) were synthesized as described above by following standard protocol. Passenger strand containing hexyl amine (HA) was conjugated with GalNAc PFP ester (Precursor 2) to form the GalNac-HA-AACAGCAAAUUCCAUCGUGA (PO to HA) conjugate.

Passenger strand (PO to HA) (70 mg, 1 equiv.) was dissolved in borate buffer (pH 8.5, 0.235 ml). GalNAc PFP ester, Precursor 2 (61 mg, 3 equiv.), was dissolved in ACN (0.2 mL). This ACN solution was added to borate buffer solution gradually and the reaction mixture was stirred. The pH of the reaction was adjusted by adding 1M aq. NaOH. After 24 h stirring, full conversion was seen by LCMS. Next, aq. ammonia (0.235 mL) was added to the reaction mixture and shaken for 6 h to hydrolyze acetyl groups. The solvent was evaporated and compound was purified by HPLC. Pure fractions were freeze dried. Obtained white solid was dissolved in 1.8 mL water and product was precipitated by using aq. NaOAc and ethanol. Centrifuged product was freeze dried to furnish sodium salt of passenger strand as a white solid. LCMS m/z ES⁻ Expected 8165.781, observed 1632.945 (z=5) Guide strand was synthesized following general synthetic protocol. Passenger strand and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. The product solution (Compound 2b) was adjusted to 1 mM in PBS buffer.

### Compound 2c:

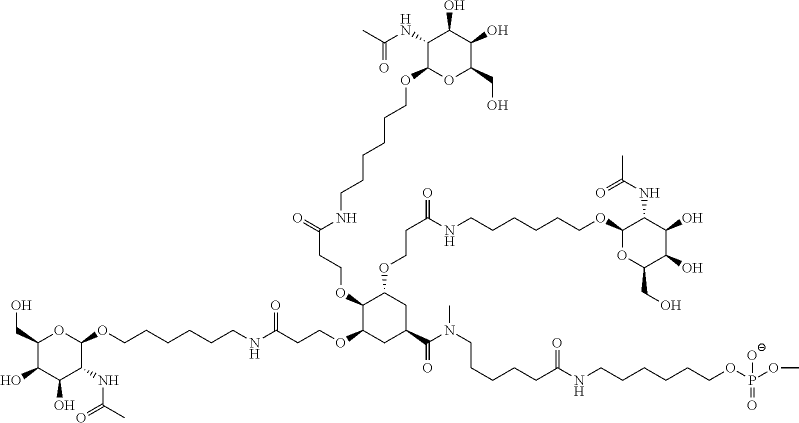 [SMILES: COP(=O)([O-])OCCCCCCNC(=O)CCCCCN(C)C(=O)[C@H]1C[C@@H](OCCC(=O)NCCCCCCO[C@@H]2O[C@H](CO)[C@H](O)[C@H](O)[C@H]2NC(C)=O)[C@@H](OCCC(=O)NCCCCCCO[C@@H]2O[C@H](CO)[C@H](O)[C@H](O)[C@H]2NC(C)=O)[C@H](OCCC(=O)NCCCCCCO[C@@H]2O[C@H](CO)[C@H](O)[C@H](O)[C@H]2NC(C)=O)C1]

MALAT1 ASO (PO to HA) (30 mg, 1 equiv.) was dissolved in borate buffer (pH 9, 0.5 mL). GalNAc PFP ester, Precursor 2 (35 mg, 4 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HIPLC. Pure fractions were freeze dried to furnish a white solid (10 mg, 25.5% , UV Purity=92.8 Area 0%). L CMS m/z ES⁻ Expected 6913.394, observed 1728.341 (z=4).

### Compound 3:

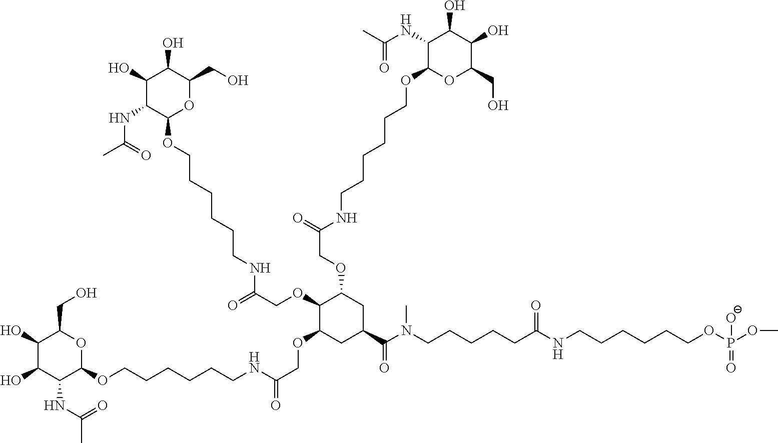 [SMILES: COP(=O)([O-])OCCCCCCNC(=O)CCCCCN(C)C(=O)[C@H]1C[C@@H](OCC(=O)NCCCCCCO[C@@H]2O[C@H](CO)[C@H](O)[C@H](O)[C@H]2NC(C)=O)[C@@H](OCC(=O)NCCCCCCO[C@@H]2O[C@H](CO)[C@H](O)[C@H](O)[C@H]2NC(C)=O)[C@H](OCC(=O)NCCCCCCO[C@@H]2O[C@H](CO)[C@H](O)[C@H](O)[C@H]2NC(C)=O)C1]

MALAT1 ASO (PO to HA) (40 mg, 1 equiv.) was dissolved in borate buffer (pH 9, 0.5 mL). GalNAc PFP ester, Precursor 3 (21 mg, 1.5 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HIPLC. Pure fractions were freeze dried to furnish a white solid (8 mg, 15.4% , UV Purity=88 Area 0%). LCMS m/z ES⁻ Expected 6871.347, observed 1717.830 (z=4).

### Compound 4:

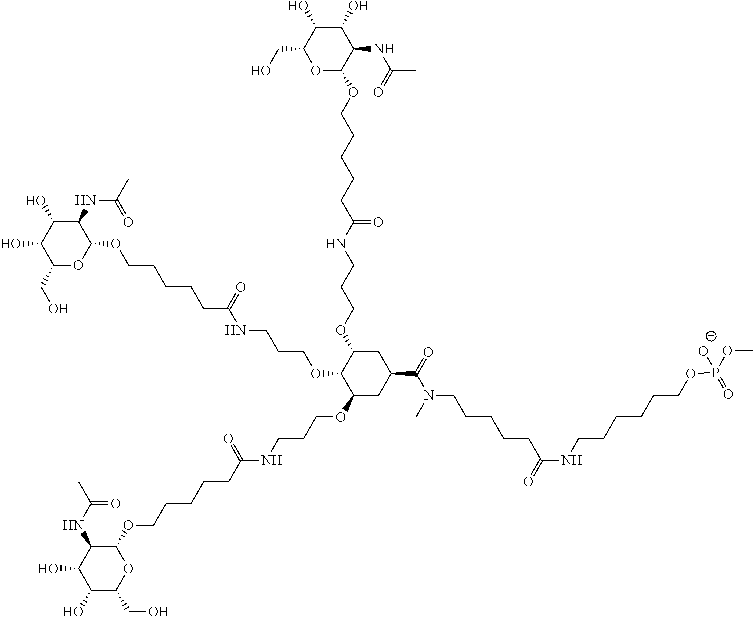 [SMILES: COP(=O)([O-])OCCCCCCNC(=O)CCCCCN(C)C(=O)[C@H]1C[C@@H](OCCCNC(=O)CCCCCO[C@@H]2O[C@H](CO)[C@H](O)[C@H](O)[C@H]2NC(C)=O)[C@H](OCCCNC(=O)CCCCCO[C@@H]2O[C@H](CO)[C@H](O)[C@H](O)[C@H]2NC(C)=O)[C@H](OCCCNC(=O)CCCCCO[C@@H]2O[C@H](CO)[C@H](O)[C@H](O)[C@H]2NC(C)=O)C1]

Compound 4 is synthesised from precursor 4 using conditions and reagents as described above.

### Compound 5:

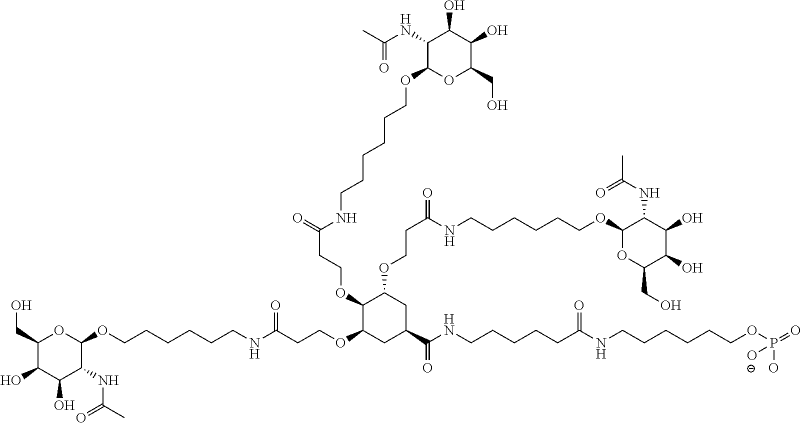 [SMILES: CC(=O)N[C@H]1[C@H](OCCCCCCNC(=O)CCO[C@@H]2C[C@H](C(=O)NCCCCCC(=O)NCCCCCCOP(=O)([O-])O)C[C@@H](OCCC(=O)NCCCCCCO[C@@H]3O[C@H](CO)[C@H](O)[C@H](O)[C@H]3NC(C)=O)[C@@H]2OCCC(=O)NCCCCCCO[C@@H]2O[C@H](CO)[C@H](O)[C@H](O)[C@H]2NC(C)=O)O[C@H](CO)[C@H](O)[C@@H]1O]

MALAT1 ASO (PO to HA) (30 mg, 1 equiv.) was dissolved in borate buffer (pH 9, 0.5 mL). GalNAc PFP ester, Precursor 5 (11 mg, 1.0 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (8 mg, 20.5%, UV Purity=88 Area %). LCMS m/z ES⁻ Expected 6899.378, observed 1724.834 (z=4).

### Compound 6:

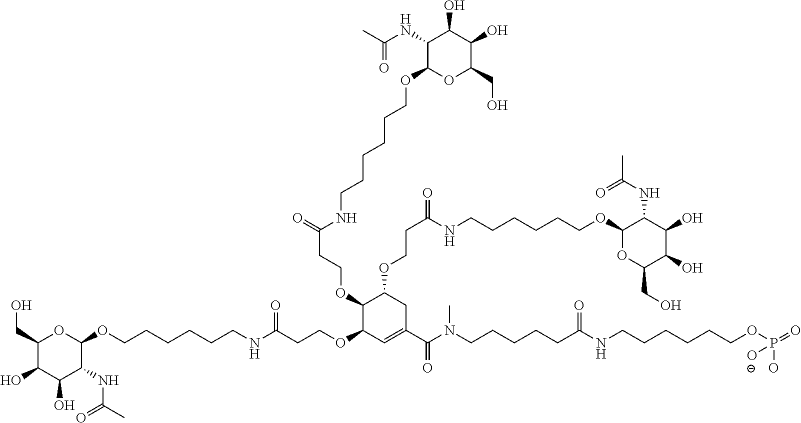 [SMILES: CC(=O)N[C@H]1[C@H](OCCCCCCNC(=O)CCO[C@@H]2[C@H](OCCC(=O)NCCCCCCO[C@@H]3O[C@H](CO)[C@H](O)[C@H](O)[C@H]3NC(C)=O)C=C(C(=O)N(C)CCCCCC(=O)NCCCCCCOP(=O)([O-])O)C[C@H]2OCCC(=O)NCCCCCCO[C@@H]2O[C@H](CO)[C@H](O)[C@H](O)[C@H]2NC(C)=O)O[C@H](CO)[C@H](O)[C@@H]1O]

MALAT1 ASO (PO to HA) (40 mg, 1 equiv.) was dissolved in borate buffer (pH 9, 1.0 mL). GalNAc PFP ester, Precursor 6 (21.5 mg, 1.5 equiv.), was dissolved in ACN (0.4 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (17 mg, 32.6%, UV Purity=96 Area %). LCMS m/z ES⁻ Expected 6911.378, observed 1727.840 (z=4).

### Compound 7:

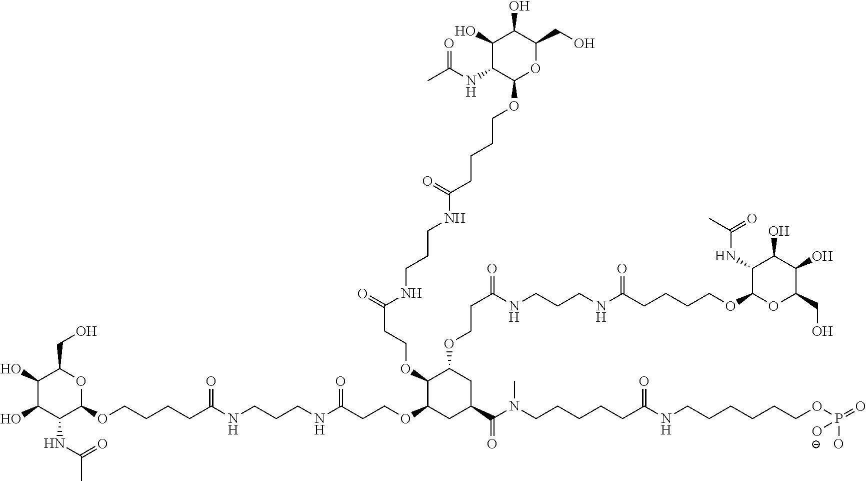 [SMILES: CC(=O)N[C@H]1[C@H](OCCCCC(=O)NCCCNC(=O)CCO[C@@H]2C[C@H](C(=O)N(C)CCCCCC(=O)NCCCCCCOP(=O)([O-])O)C[C@@H](OCCC(=O)NCCCNC(=O)CCCCO[C@@H]3O[C@H](CO)[C@H](O)[C@H](O)[C@H]3NC(C)=O)[C@@H]2OCCC(=O)NCCCNC(=O)CCCCO[C@@H]2O[C@H](CO)[C@H](O)[C@H](O)[C@H]2NC(C)=O)O[C@H](CO)[C@H](O)[C@@H]1O]

MALAT1 ASO (PO to HA) (40 mg, 1 equiv.) was dissolved in borate buffer (pH 9, 1.0 mL). GalNAc PFP ester, Precursor 7 (23.4 mg, 1.5 equiv.), was dissolved in ACN (0.4 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (13 mg, 24.3%, UV Purity=92 Area %). LCMS m/z ES⁻ Expected 7084.458, observed 1771.109 (z=4).

### Compound 8:

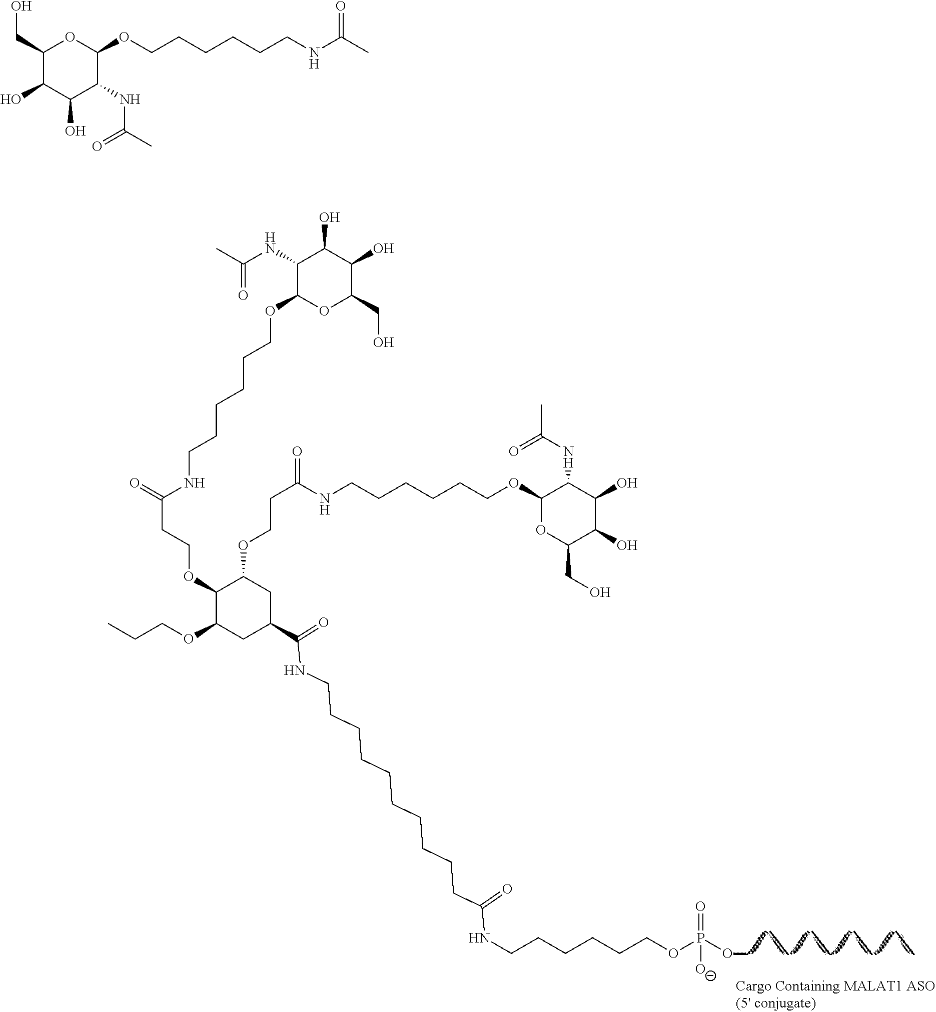 [SMILES: CC(=O)NCCCCCCO[C@@H]1O[C@H](CO)[C@H](O)[C@H](O)[C@H]1NC(C)=O ; CCCO[C@@H]1C[C@H](C(=O)NCCCCCCCCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 8 (17.7 mg, 1.2 equiv.), was dissolved in ACN (0.2 mL). This ACN solution was added to borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (12 mg, 22.8%, UV Purity=96 Area %). LCMS m/z ES⁻ Expected 6969.456, observed 1742.359 (z=4).

### Compound 9:

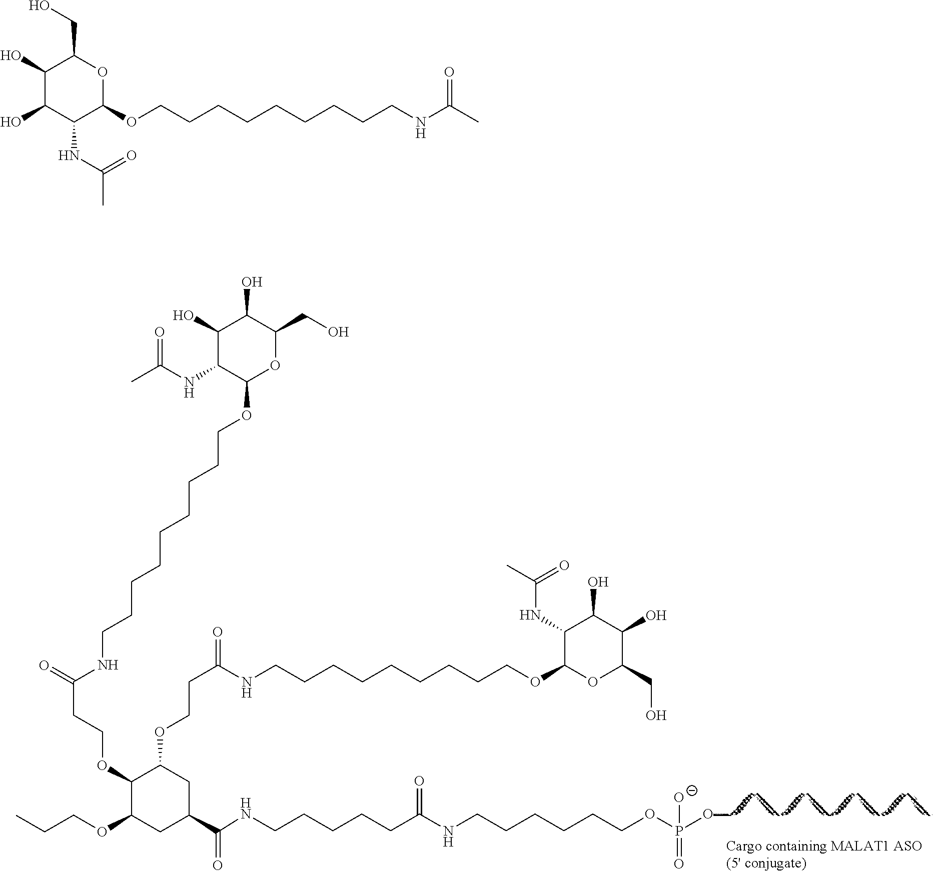

MALAT1 ASO (PO to HA) (40 mg, 1 equiv.) was dissolved in borate buffer (pH 9, 0.8 mL). GalNAc PFP ester, Precursor 9 (30 mg, 2 equiv.), was dissolved in ACN (0.4 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (15 mg, 28.3%, UV Purity=96 Area %). LCMS m/z ES⁻ Expected 7025.519, observed 1756.376 (z=4).

### Compound 10:

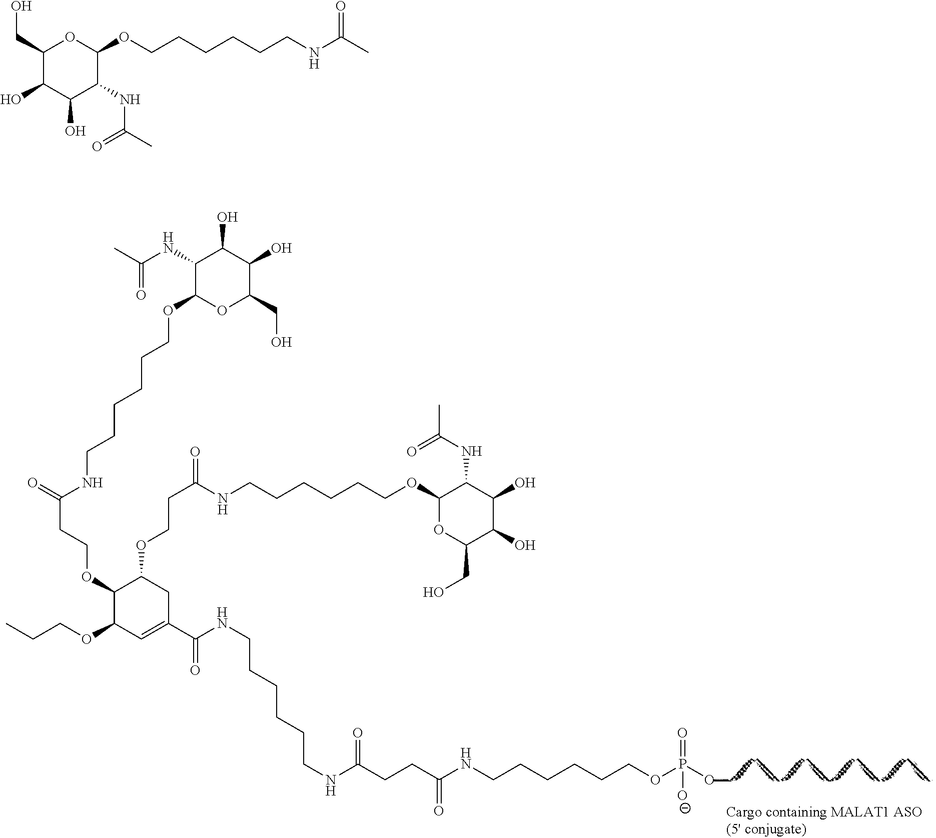 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCCCNC(=O)CCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 10 (22 mg, 1.5 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (15 mg, 28.3%, UV Purity=96 Area %). LCMS m/z ES⁻ Expected 7025.519, observed 1756.376 (z=4).

### Compound 11:

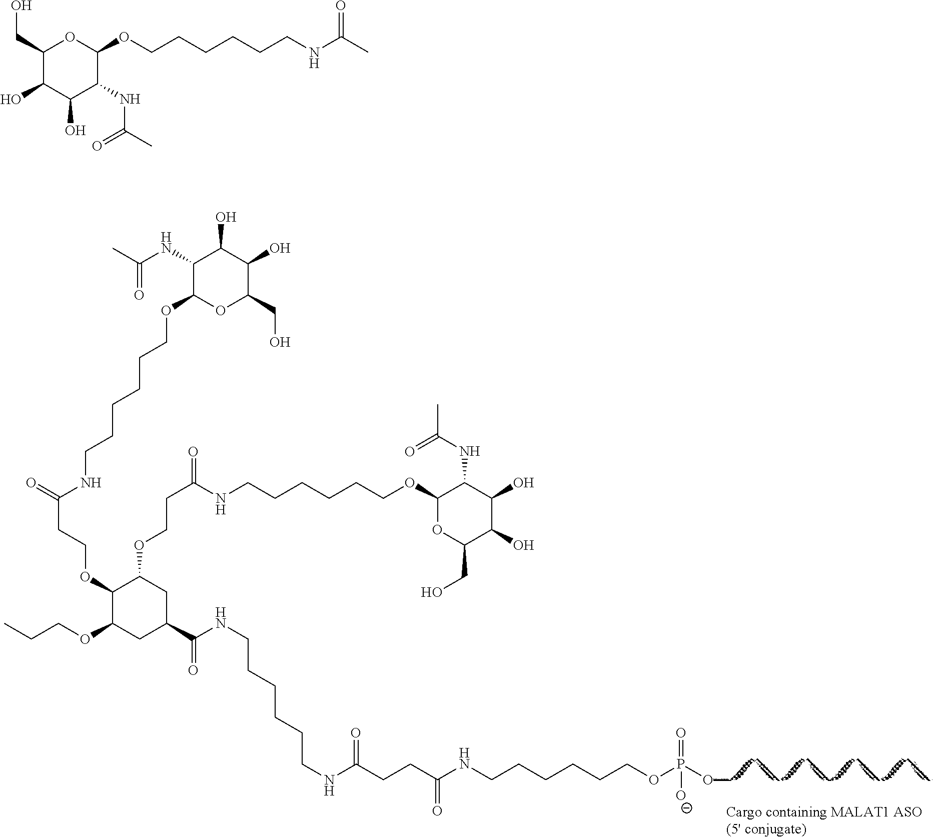 [SMILES: CC(=O)NCCCCCCO[C@@H]1O[C@H](CO)[C@H](O)[C@H](O)[C@H]1NC(C)=O ; CCCO[C@@H]1C[C@H](C(=O)NCCCCCCNC(=O)CCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 11 (17.8 mg, 1.2 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (8 mg, 15%, UV Purity=97 Area %). LCMS m/z ES⁻ Expected 6984.431, observed 1746.1030 (z=4).

### Compound 12:

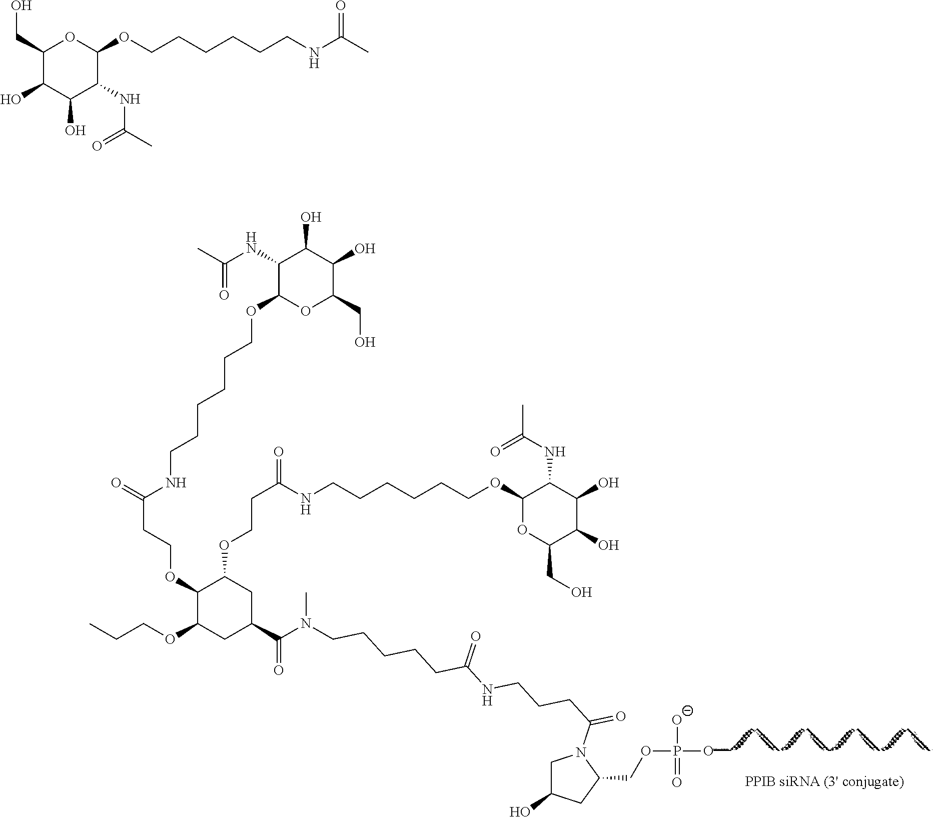 [SMILES: CC(=O)NCCCCCCO[C@@H]1O[C@H](CO)[C@H](O)[C@H](O)[C@H]1NC(C)=O ; CCCO[C@@H]1C[C@H](C(=O)N(C)CCCCCC(=O)NCCCC(=O)N2C[C@H](O)C[C@H]2COP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

PPIB passenger stand (18-mer, SEQ ID NO: 4) was synthesized on solid support derived from 12-5 using the general synthesis protocol described above. After completing the synthesis on oligo synthesizer, the solid support was treated with methyl amine gas to hydrolyze the passenger strand from the solid support. The solid support was washed with a mixture of NaOAc 0.1 M in EtOH (85%), followed by EtOH (85%) and finally eluted using water. The combined fractions were concentrated by Speedvac and the compound was purified by HPLC. Pure fractions were freeze dried to afford a white solid. The white solid was dissolved in 0.3 mL water and product was precipitated by using aq. NaOAc and ethanol. Centrifuged product was freeze dried to furnish the sodium salt of the passenger strand as a white solid. LCMS m/z ES⁻ Expected 7576.681, observed 1893.916 (z=4)

The guide strand (SEQ ID NO: 6) was synthesized following the general synthetic protocol. The passenger strand and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. The product solution (Compound 12) was adjusted to 1 mM in PBS buffer.

### Compound 13:

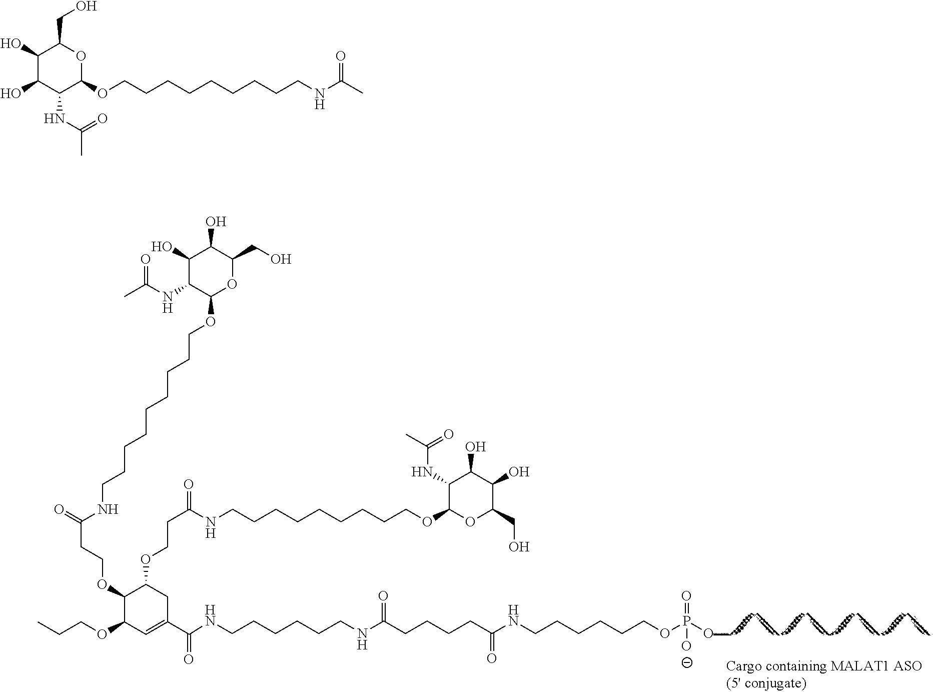 [SMILES: CC(=O)NCCCCCCCCCO[C@@H]1O[C@H](CO)[C@H](O)[C@H](O)[C@H]1NC(C)=O ; CCCO[C@@H]1C=C(C(=O)NCCCCCCNC(=O)CCCCC(=O)NCCCCCCOP(=O)([O-])O)C[C@@H](OCCC(=O)NCCCCCCCCCO[C@@H]2O[C@H](CO)[C@H](O)[C@H](O)[C@H]2NC(C)=O)[C@@H]1OCCC(=O)NCCCCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 13 (48 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (12 mg, 23% UV Purity=97.5 Area %). LCMS m/z ES⁻ Expected 7136.587, observed 1784.141 (z=4).

### Compound 14:

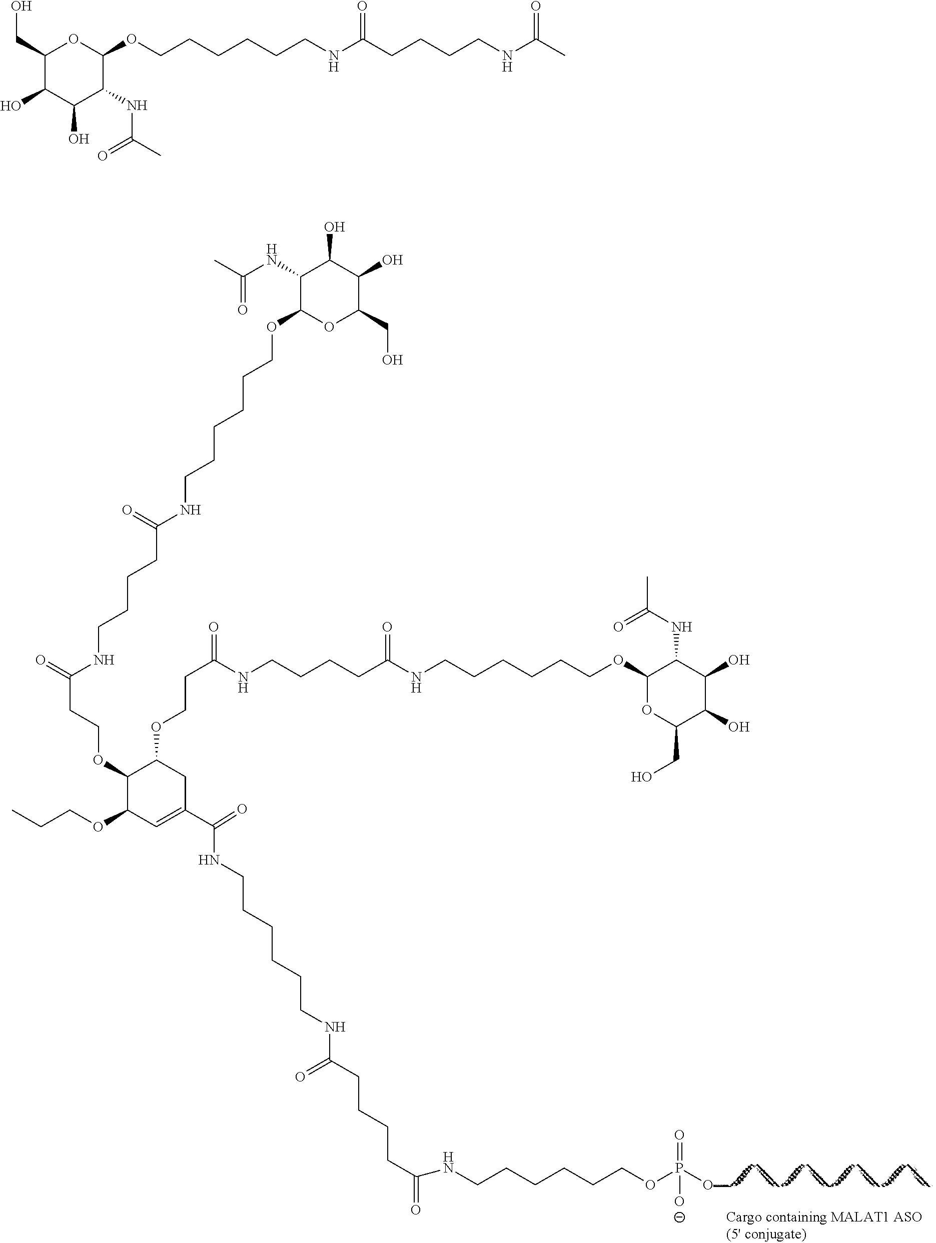

MALAT1 ASO (PO to HA) (40 mg, 1 equiv.) was dissolved in borate buffer (pH 9, 0.5 mL). GalNAc PFP ester, Precursor 14 (52 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (13 mg, 25%, UV Purity=98 Area %). LCMS m/z ES⁻ Expected 7307.652, observed 1826.903 (z=4).

### Compound 15a:

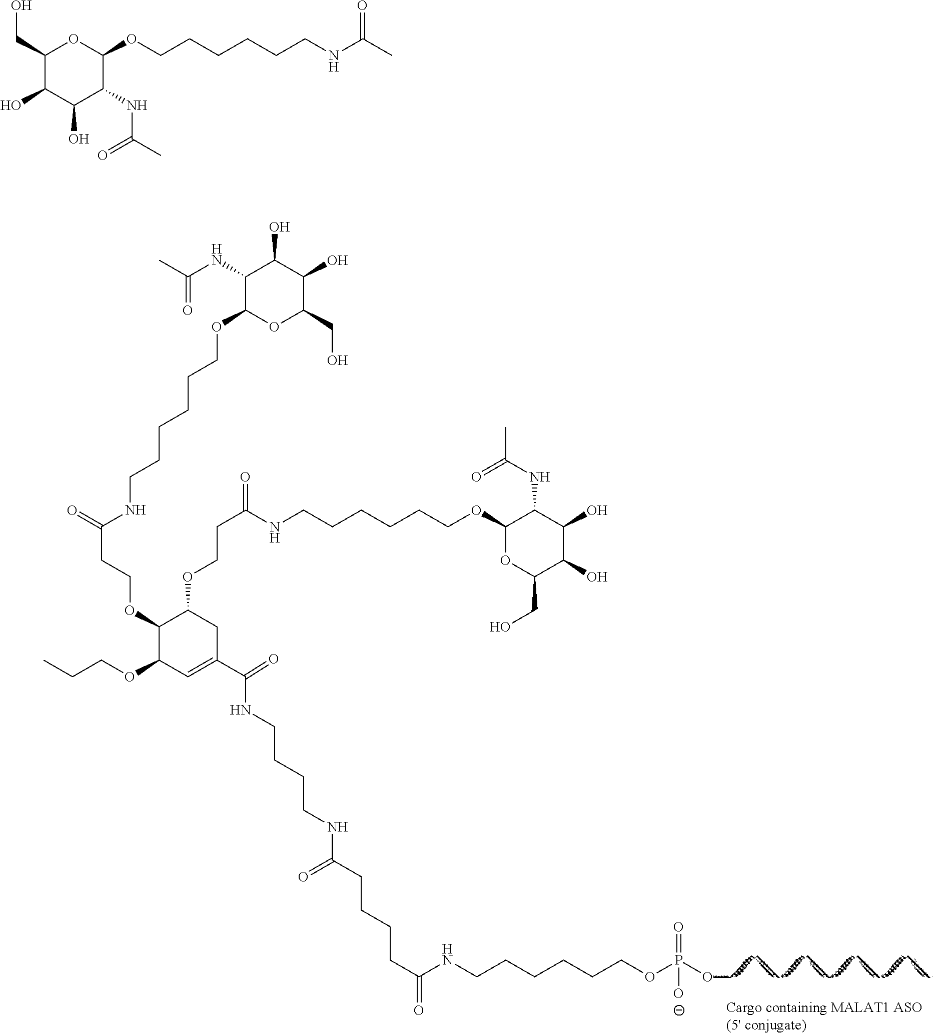 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCNC(=O)CCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 15 (45 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (8 mg, 15%, UV Purity=96 Area %). LCMS m/z ES⁻ Expected 6982.415, observed 1745.589 (z=4).

### Compound 15b:

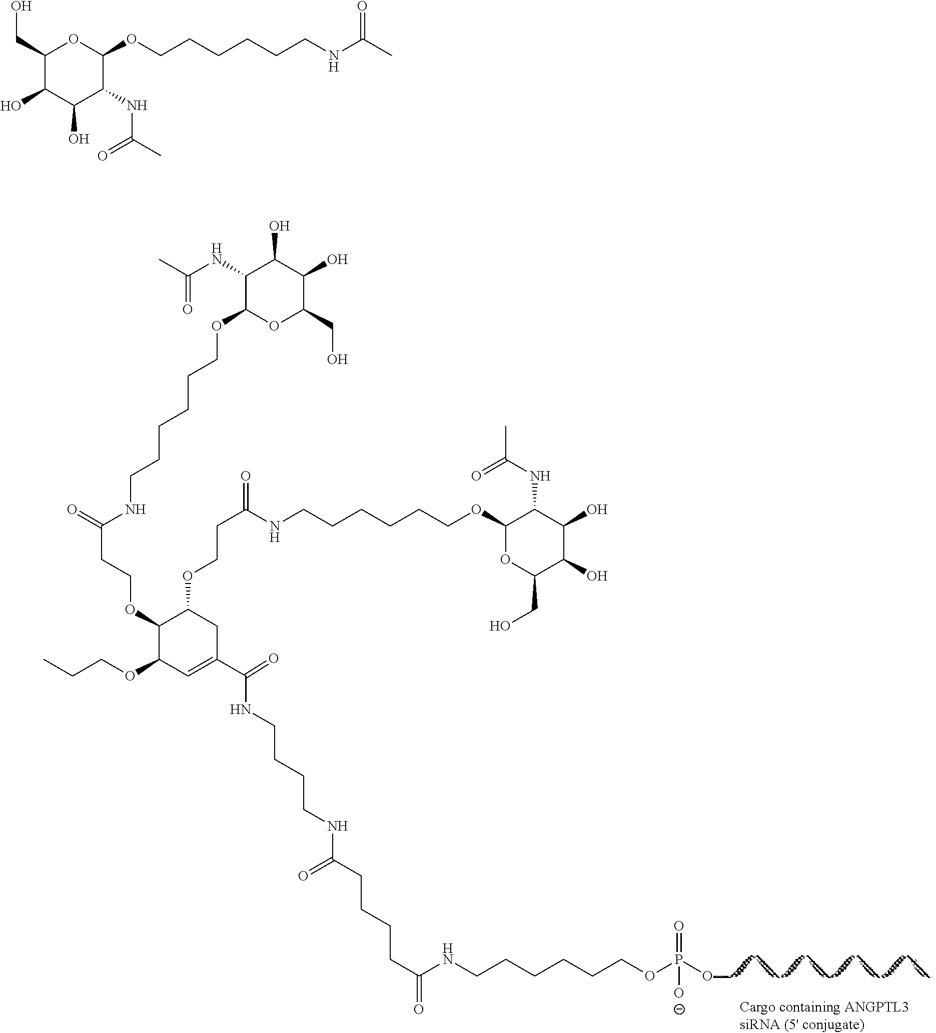 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCNC(=O)CCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

Passenger and guide strands were synthesized as described above. Passenger strand was conjugated with GalNAc to form the GalNac-HA-invAb*gcucaacaUAUuugaucagua*invAb (PO to HA) complex.

Passenger strand (PO to HA) (20 mg, 1 equiv.) was dissolved in borate buffer (pH 9, 0.25 ml). GalNAc PFP ester, Precursor 15 (17 mg, 3 equiv.), was dissolved in ACN (0.1 mL). This ACN solution was added to borate buffer solution gradually and the reaction mixture was stirred. After 2 h, full conversion was seen by LCMS. Next, aq. ammonia (1 mL) was added to the reaction mixture and shaken overnight to hydrolyze acetyl groups. The solvent was evaporated and compound was purified by HPLC. Pure fractions were freeze dried. Obtained white solid (8 mg, 33%) was dissolved in 0.3 mL water and product was precipitated by using aq. NaOAc and ethanol. Centrifuged product was freeze dried to furnish sodium salt of passenger strand as a white solid. LCMS m/z ES⁻ Expected 8945.023, observed 2235.216 (z=4).

Guide strand was synthesized following general synthetic protocol. Passenger strand and guide strands were dissolved in PBS buffer separately to make equimolar solutions of each strand. Both the solutions were mixed. Next, following standard conditions (5 min at 95° C., then slow cooling down to 20° C. for 1 h with gentle shaking) annealing was performed. QC gel was performed for analysis. Product solution (Compound 15b) was adjusted to 1 mM in PBS buffer.

### Compound 16a:

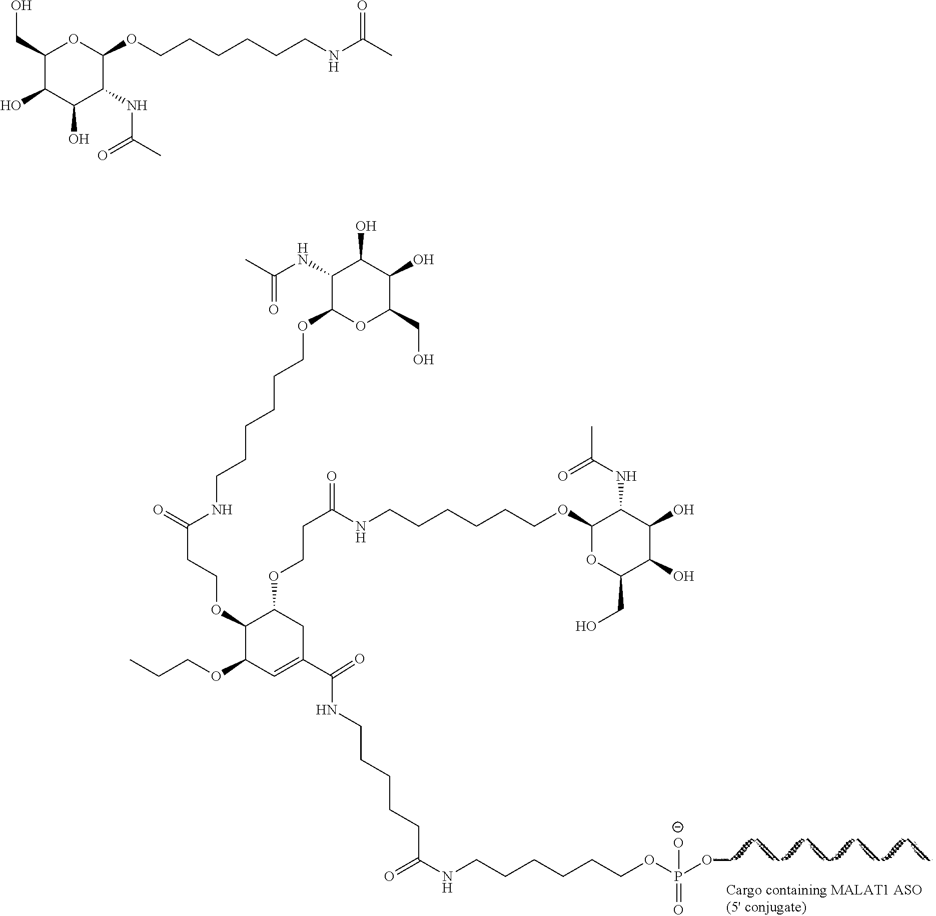 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 16 (43 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (9 mg, 17%, UV Purity=94 Area %). LCMS m/z ES⁻ Expected 6897.362, observed 1724.324 (z=4).

### Compound 16b:

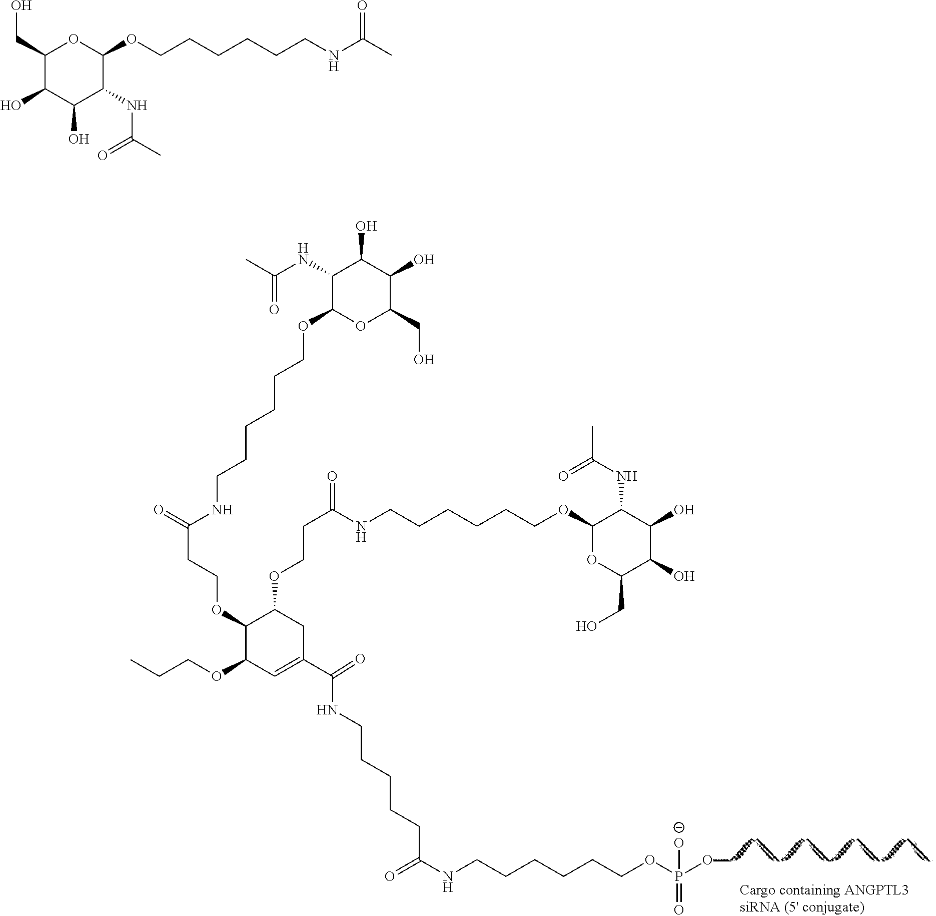 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

Passenger and guide strands were synthesized as described above. Passenger strand was conjugated with GalNAc to form the GalNac-HA-invAb*gcucaacaUAUuugaucagua*invAb (PO to HA) complex.

Passenger strand (PO to HA) (20 mg, 1 equiv.) was dissolved in borate buffer (pH 9, 0.25 ml). GalNAc PFP ester, Precursor 16 (17 mg, 3 equiv.), was dissolved in ACN (0.1 mL). This ACN solution was added to borate buffer solution gradually and the reaction mixture was stirred. After 2 h, full conversion was seen by LCMS. Next, aq. ammonia (1 mL) was added to the reaction mixture and shaken overnight to hydrolyze acetyl groups. The solvent was evaporated and compound was purified by HPLC. Pure fractions were freeze dried. Obtained white solid (9 mg, 37%) was dissolved in 0.3 mL water and product was precipitated by using aq. NaOAc and ethanol. Centrifuged product was freeze dried to furnish sodium salt of passenger strand as a white solid. LCMS m/z ES⁻ Expected 9037.720, observed 2258.108 (z=4).

Guide strand was synthesized following general synthetic protocol. Passenger strand and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. Product solution (Compound 16b) was adjusted to 1 mM in PBS buffer.

### Compound 17:

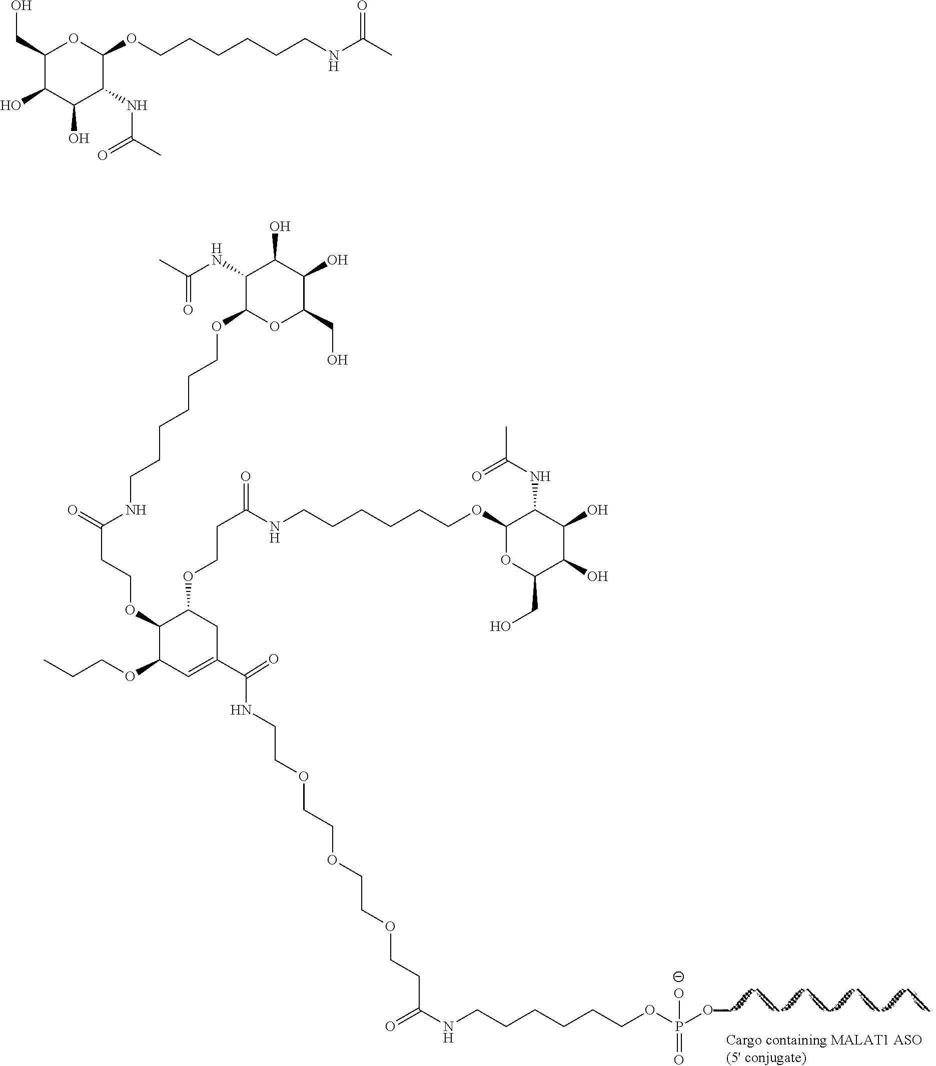 [SMILES: CC(=O)NCCCCCCO[C@@H]1O[C@H](CO)[C@H](O)[C@H](O)[C@H]1NC(C)=O ; CCCO[C@@H]1C=C(C(=O)NCCOCCOCCOCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (40 mg, 1 equiv.) was dissolved in borate buffer (pH 9, 0.5 mL). GalNAc PFP ester, Precursor 17 (45 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (6 mg, 12%, UV Purity=90 Area %). LCMS m/z ES⁻ Expected 6987.394, observed 1745.837 (z=4).

### Compound 18:

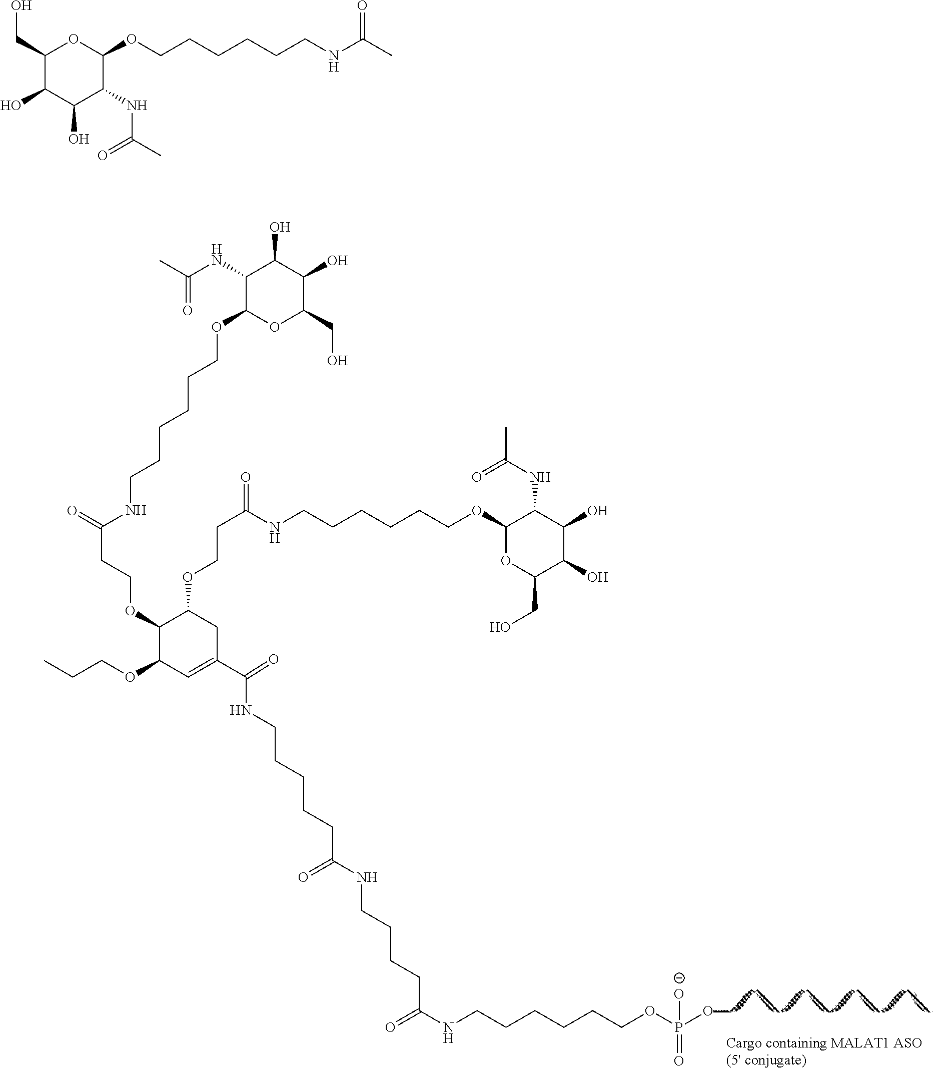 [SMILES: CC(=O)NCCCCCCO[C@@H]1O[C@H](CO)[C@H](O)[C@H](O)[C@H]1NC(C)=O ; CCCO[C@@H]1C=C(C(=O)NCCCCCC(=O)NCCCCC(=O)NCCCCCCOP(=O)([O-])O)C[C@@H](OCCC(=O)NCCCCCCO[C@@H]2O[C@H](CO)[C@H](O)[C@H](O)[C@H]2NC(C)=O)[C@@H]1OCCC(=O)NCCCCCCO[C@@H]1O[C@H](CO)[C@H](O)[C@H](O)[C@H]1NC(C)=O]

MALAT1 ASO (PO to HA) (45 mg, 1 equiv.) was dissolved in borate buffer (pH 9, 0.5 mL). GalNAc PFP ester, Precursor 18 (40 mg, 3 equiv.), was dissolved in ACN (0.2 mL). This ACN solution was added to the borate buffer solution gradually and the reaction mixture was stirred. After 20 h, full conversion was seen by LCMS. Next, aq. ammonia (1 mL) was added to the reaction mixture and shaken overnight to hydrolyze acetyl groups. The solvent was evaporated and the compound was purified by HPLC. Pure fractions were freeze dried to furnish a white solid (9 mg, 18%, UV Purity=94 Area %). LCMS m/z ES⁻ Expected 6996.431, observed 1748.096 (z=4).

### Compound 19:

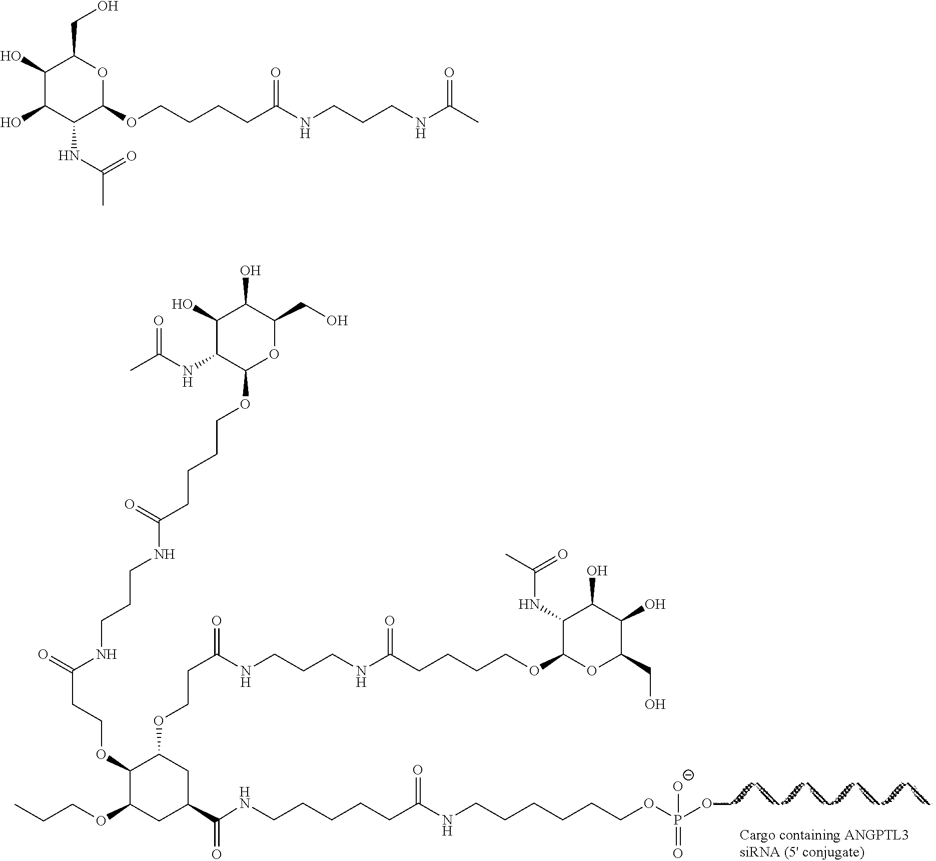

Passenger and guide strands were synthesized as described above. Passenger strand was conjugated with GalNAc to form the GalNac-HA-invAb*gcucaacaUAUuugaucagua*invAb (PO to HA) complex.

Passenger strand (PO to HA) (20 mg, 1 equiv.) was dissolved in borate buffer (pH 9, 0.25 ml). GalNAc PFP ester, Precursor 19 (16 mg, 3 equiv.), was dissolved in ACN (0.1 mL). This ACN solution was added to borate buffer solution gradually and the reaction mixture was stirred. After 2 h, full conversion was seen by LCMS. Next, aq. ammonia (1 mL) was added to the reaction mixture and shaken overnight to hydrolyze acetyl groups. The solvent was evaporated and compound was purified by HPLC. Pure fractions were freeze dried. Obtained white solid (10 mg, 42%) was dissolved in 0.3 mL water and product was precipitated by using aq. NaOAc and ethanol. Centrifuged product was freeze dried to furnish sodium salt of passenger strand as a white solid. LCMS m/z ES⁻ Expected 8864.548, observed 2215.002 (z=4).

Guide strand was synthesized following general synthetic protocol. Passenger strand and guide strands were dissolved in PBS buffer separately to make equimolar solutions of each strands. Both the solutions were mixed. Next, following standard conditions (5 min at 95° C., then slow cooling down to 20° C. for 1 h with gentle shaking) annealing was performed. QC gel was performed for analysis. Product solution (Compound 19) was adjusted to 1 mM in PBS buffer.

### Compound 20:

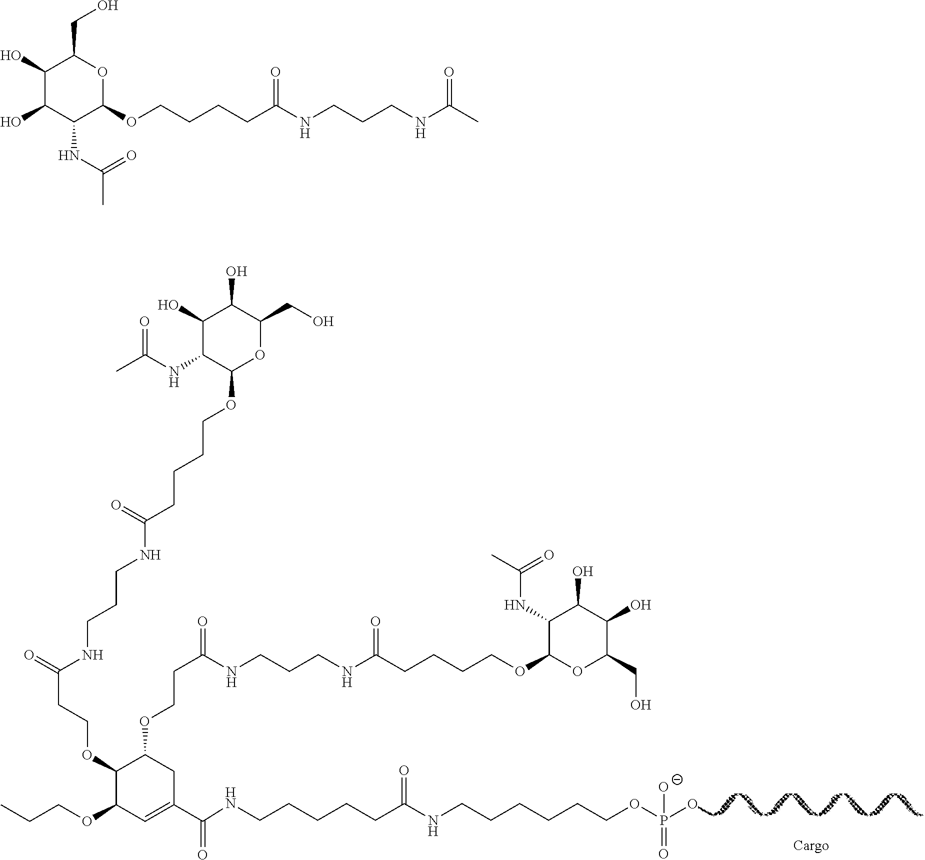

Compound 20 is synthesised from precursor 20 using conditions and reagents as described above.

### Compound 21:

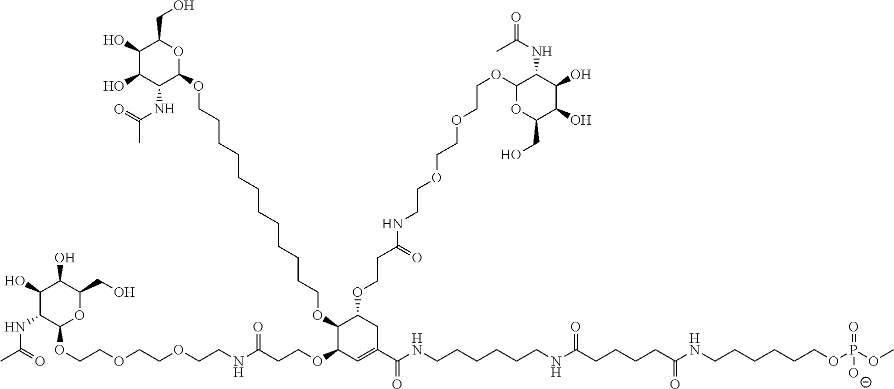 [SMILES: COP(=O)([O-])OCCCCCCNC(=O)CCCCC(=O)NCCCCCCNC(=O)C1=C[C@@H](OCCC(=O)NCCOCCOCCO[C@@H]2O[C@H](CO)[C@H](O)[C@H](O)[C@H]2NC(C)=O)[C@@H](OCCCCCCCCCCCCO[C@@H]2O[C@H](CO)[C@H](O)[C@H](O)[C@H]2NC(C)=O)[C@H](OCCC(=O)NCCOCCOCCOC2O[C@H](CO)[C@H](O)[C@H](O)[C@H]2NC(C)=O)C1]

Compound 21 is synthesised from precursor 21 using conditions and reagents as described above.

Control ASO/siRNA cluster: triantennary/amide spacers and prolinol linker

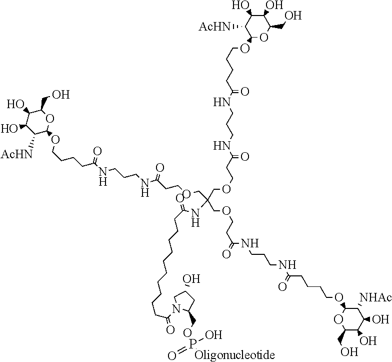

Reference compounds (control cluster coupled to MALAT1 ASO and ANGPTL3/PPIB siRNA) were synthesized by an analogous protocol to that described above.

### Example 2—In Vitro Biological Assays

Several assays were performed to assess the activity of compounds of the Examples in vitro as compared to control compounds (e.g., the naked MALAT1 ASO).

Human ASGPR1 HTRF binding assay (“FRET IC₅₀” assay)

A binding assay using HTRF was performed to determine the binding affinities of ASGPR ligands.

HTRF (Homogeneous Time Resolved Fluorescence) assay is a no-wash technology using standard FRET technology with time-resolved measurement of fluorescence. In the human ASGPR1 HTRF assay, HIS-tagged human trimeric ASGPR1 protein and a tri-antennary Alexa-647 labelled probe are used. Upon adding detection reagents containing Terbium-labelled anti-HIS-antibody, a TR-FRET signal can be generated and measured. An ASGPR1 ligand can compete with Alexa-647 labelled probe for binding to the ASGPR1 protein and thus lead to decreased TR-FRET signal ratio (Em665/Em620).

The human ASGPR1 HTRF binding assay was performed in 384-well microplates (Greiner, #784075). Recombinant human ASGPR1 protein with a HIS-tag was expressed in mammalian cells and purified via chromatography. Anti-HIS-antibody was coupled to a donor (Terbium in this case) and Alexa-647 fluorophore-labelled trivalent GalNAc—tool compound was used as an acceptor. In the human ASGPR1 HTRF binding experiments, 100 nL/well of compounds were dispended to 384 well assay plates, 5 μL of solution 1 containing 20 nM of HIS-tagged recombinant human trimeric ASGPR1 protein was added to wells and mixed well. After 15 min incubation at room temperature, 5 μL of solution 2 containing 10 nM of Alexa-647-labelled trivalent GalNAc—tool compound and 0.2 μg/mL of Terbium-labelled anti-HIS-antibody were added. After 60 min incubation at room temperature, the HTRF signals were measured in a PHERAstar FSX plate reader with a HTRF module. The optimal HTRF binding assay buffer was 50 mM Tris-C1 (pH7.5), 20 mM CaCl₂), 0.01% Pluronic F-127. IC₅₀(Half maximal inhibitory concentration) values were calculated based on 10 concentration response curves.

### Human ASGPR1 Surface Plasmon Resonance Binding Assay

A binding assay using SPR was performed to determine the binding affinities and kinetics of ASGPR ligands.

Surface plasmon resonance (SPR) binding assay use surface immobilized Asialoglycoprotein Receptor Protein, ASGPR1(148-291). Immobilization of the biotinylated 6His-Avi-TEV-ASGPR1(148-291) is performed by direct capture onto a streptavidin-derivatized dextran sensor chip. Typical immobilization level is 4000-8000 RU. Reference surface is left untreated. Test compounds are diluted in assay buffer (50 mM Tris pH 7.4, 150 mM NaCl, 50 mM CaCl₂, 0-1% DMSO) and injected over the sensor surface to generate concentration response curves. Sensorgrams are fitted to a 1:1 binding model to determine affinities and binding kinetics. All data was generated using an 8K or S200 Biacore (Cytiva).

### Mouse ASGPR Fluorescent Polarization Binding Assay (“Microsome FP IC₅₀” Assay)

The mouse ASGPR FP (Fluorescent polarization) assay was used to measure the binding affinities of ASGPR ligands to native ASGPR in mouse hepatocytes membrane. The mouse ASGPR FP assay was performed in 384-well microplates (Greiner, #781076). The membrane prepared from C57BL/6 mice livers was used as a source of ASGPR and Alexa-647 fluorophore-labelled trivalent GalNAc—tool compound was used as tracer.

In the FP binding experiments, 100 nL of compounds was mixed with 5 μL of solution A containing 4 μg of mouse liver membrane. After 15 min incubation at room temperature, 5 μL of solution B containing Alexa-647—labelled trivalent GalNAc—tool compound (final concentration 0.5 nM) were added. After 60 min incubation at room temperature, the FP signals were measured in a PHERAstar FSX plate reader with a FP optical module (FP 590/575/575). The optimal FP assay buffer was 50 mM Tris-C1 of pH7.5, 3 mM CaCl₂), 0.04% Triton X-100. IC₅₀ (Half maximal inhibitory concentration) values were calculated based on 10 concentration response curves.

Human ASGPR qPCR Assay

A qPCR (quantitative polymerase chain reaction) assay was applied to measure mRNA levels of the target sequence MALAT1, which is in conjugate with the ASGPR1 tri-antennary ligand. This is done in HEK293 cells overexpressing the human ASGPR1 subunit. The assay was performed in PDL-coated 384-well microplates, prepared with conjugates (10 μM final top concentration with a 1:3 dilution in 10-point dose response) using Echo dispensing. Cells in DMEM GlutMax media (#31966, supplemented with 10% FBS) were seeded ontop for 48h incubation. Medium was removed, cells lysed and cRNA was prepared from the resulting lysates by reverse transcription. cDNA is diluted 1:4 and finally quantitative PCR (qPCR) by Malat1 Taqman assay is run to analyse target expression. Rack1 Taqman assay is included as an internal control of housekeeping gene expression. Dose response is normalized to 100% inhibition control: naked Malat1 (10 μM) and 0% control: water.

Human hepatocytes were obtained from a commercial supplier (BioIVT, QNT lot) and cultured in rat tail collagen I coated plates. The hepatocytes were maintained in William's E Medium, supplemented with hepatocyte supplemented media and 5C supplements (DAPT, SB431542, Forskolin, IWP2 and LDN193189). The cells were cultured at 37° C. in a humidified incubator with 5% CO₂. Next after plating (Day 1), cells were treated with different siRNA conjugates concentrations ranging from 10 μM to 0.01 nM and PBS in maintenance media. After 24 h of treatment, media was changed and cells were harvested on Day 4 (72 h from start of the treatment) for qPCR. Total RNA was extracted according to standard protocol setup in the lab. cDNA synthesis and qPCR were also run according to standard validated protocol. ANGPLT3, PPIB and GAPDH TaqMan primers were purchased from Thermo Fischer. The half maximal inhibitory concentration (ICSO) for each siRNA was calculated using GraphPad Prism 8 software.

The results of the above assays for exemplary compounds are shown in Tables 1 and 2 below:

TABLE 1
Assay results for exemplary ASO compounds

| | | | | HEK | |
|---|---|---|---|---|---|
| | | | Microsome | ASGPR1 | |
| Compound | FRET IC₅₀ | SPR Kd | FP IC₅₀ | qPCR IC₅₀ | |
| Naked MALAT1 | — | Inactive | Inactive | — | |
| ASO | | | | | |
| Compound 1 | 3.4 nM | 1.1 nM | 2.0 nM | 339 | nM |
| Compound 2c | 1.8 nM | 2.5 nM | 2.0 nM | 47.1 | nM |
| Compound 3 | 1.3 nM | 5.0 nM | 2.6 nM | 42.3 | nM |
| Compound 5 | 1.2 nM | 6.6 nM | 1.0 nM | 9.6 | nM |
| Compound 6 | 2.4 nM | 3.9 nM | 2.6 nM | 27.2 | nM |
| Compound 7 | 1.4 nM | 4.3 nM | 1.2 nM | 17.5 | nM |
| Compound 8 | 2.1 nM | 2.5 nM | 2.5 nM | 84.6 | nM |
| Compound 9 | 0.8 nM | 2.5 nM | 0.4 nM | 56.5 | nM |
| Compound 10 | 2.7 nM | 6.5 nM | 2.8 nM | 25.7 | nM |
| Compound 11 | 1.1 nM | 2.2 nM | 2.2 nM | 34.8 | nM |
| Compound 15a | 1.6 nM | 3.2 nM | 2.5 nM | 14.4 | nM |
| Compound 16a | 0.6 nM | 7.7 nM | 1.0 nM | 8.0 | nM |

“—” denotes that the Compound was not tested

TABLE 2
Assay results for exemplary siRNA compounds

| | FRET | | | Microsome | | PHH ASGPR1 | |
|---|---|---|---|---|---|---|---|
| Compound | IC₅₀ | SPR Kd | | FP IC₅₀ | | qPCR IC₅₀ | |
| Control siRNA | 1.6 nM | 2.6 | nM | 6.1 | nM | 10 | nM |
| cluster (ANGPTL3) | | | | | | | |
| Control siRNA | 2.6 nM | 13.3 | nM | 3.4 | nM | 10 | nM |
| cluster (PPIB) | | | | | | | |
| Compound 2a | — | 1.7 | nM | 5.34 | nM | 8 | nM |
| (ANGPTL3) | | | | | | | |
| Compound 2b | 3.1 nM | 10.9 | nM | 6.6 | nM | 4 | nM |
| (PPIB) | | | | | | | |
| Compound 12 | 2.7 nM | 15.9 | nM | 5.1 | nM | 10.0 | nM |
| (PPIB) | | | | | | | |
| Compound 15b | 1.4 nM | 7.1 | nM | 1.7 | nM | 4 | nM |
| (ANGPTL3) | | | | | | | |
| Compound 16b | 2.4 nM | 15.3 | nM | 6.9 | nM | 10 | nM |
| (ANGPTL3) | | | | | | | |
| Compound 19 | 1.5 nM | 44.9 | nM | 2.7 | nM | 20 | nM |
| (ANGPTL3) | | | | | | | |

“—” denotes that the Compound was not tested
PHH denotes primary human hepatocytes

The data in Tables 1 and 2 indicate that the tested compounds are active, and improved vis-?i-vis control nucleic acid. Naked MALAT1 nucleic acid was inactive in SPR and FP assays while the compound of Example 1 (and the other ASO-containing compounds) showed potency at single digit nM levels in the FRET, SPR and Microsome assays. The siRNA-containing complexes were also active in the single digit nM range in the FRET, SPR and Microsome assays. Activity was observed for all tested compounds in the ASGPR1 qPCR assays.

### Example 3—In Vivo Biological Assay

ASO knockdown of MALAT1 in mice

Compounds were tested in vivo for knockdown of MALAT-1.

Animals were housed in microisolator cages on a constant 12-hour light/dark cycle with controlled temperature and humidity and with access to normal chow and water ad libitum. All in vivo experiments were performed in accordance with AstraZeneca Institutional Animal Care and Use Committee guidelines. C57BL/6 mice (male 25-27 g; Taconic or Charles River) were treated subcutaneously or intravenously with a single dose of 0.01 umol/kg, 0.05 umol/kg, or 0.25 umol/kg, ASO or PBS (mock control) in groups of n=3. Blood samples were drawn 72h post-dosing and animals terminated. The inhibition of ASO target MALAT1 gene expression was quantified by real-time PCR in liver and kidney tissue samples. In addition, plasma samples to measure ALT were performed by biochemical analyzer.

Total RNA was extracted from cells or tissue samples using the RNeasy 96 QIAcube HT Kit (Qiagen). Complementary DNA (cDNA) was generated using a High-Capacity cDNA Reverse Transcription kit (Applied Biosystems). Reverse transcription qPCR analysis was performed with a QuantStudio 7 Flex using MALAT1 TaqMan assays (Mouse Mm01227912, Applied Biosystems) following the manufacturer's instructions.

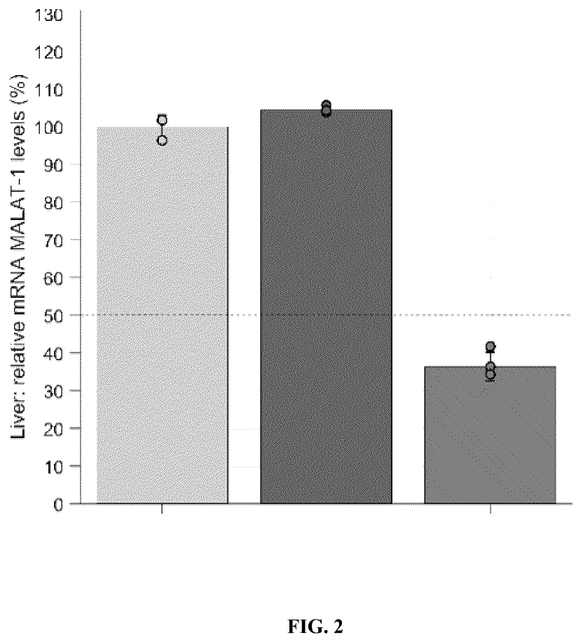

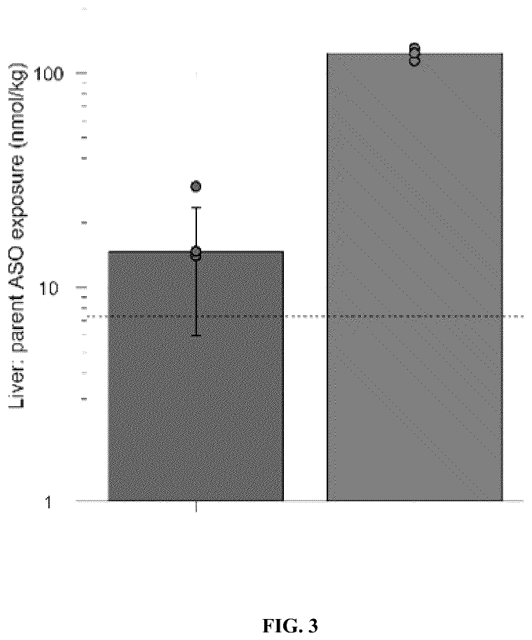

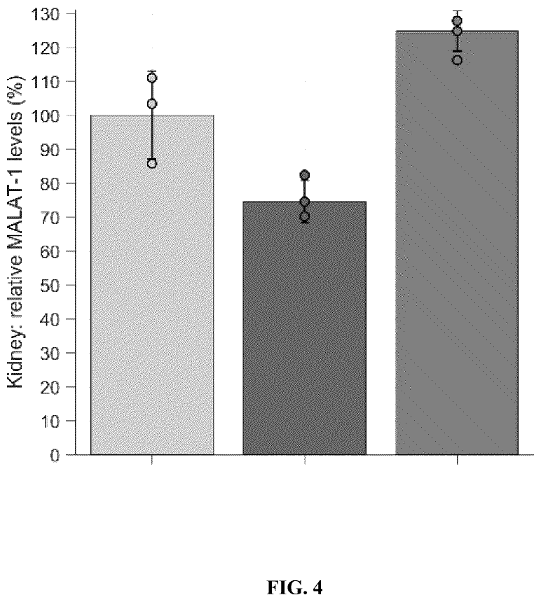

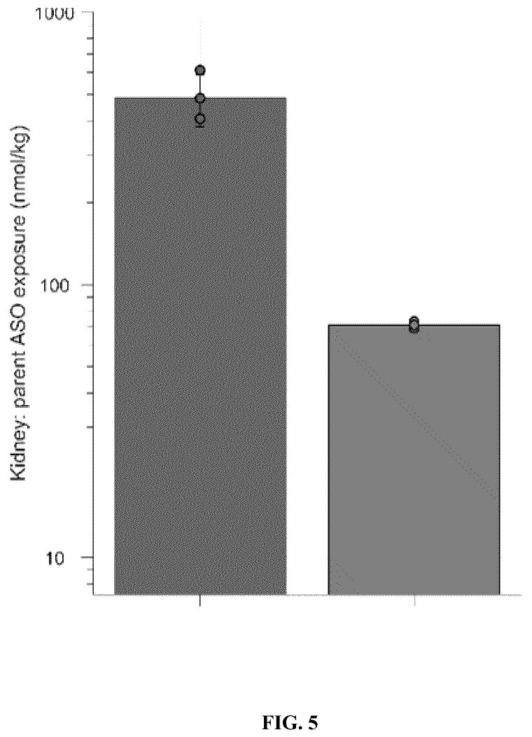

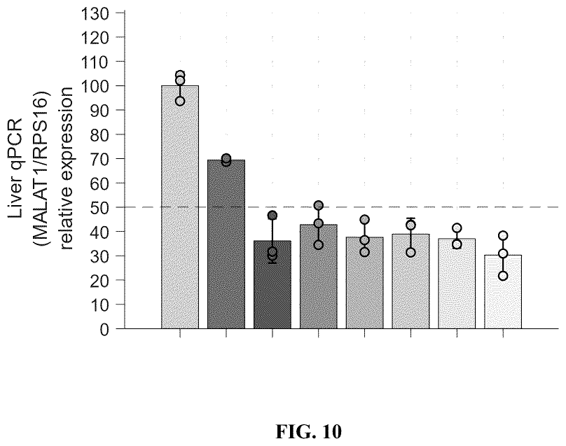

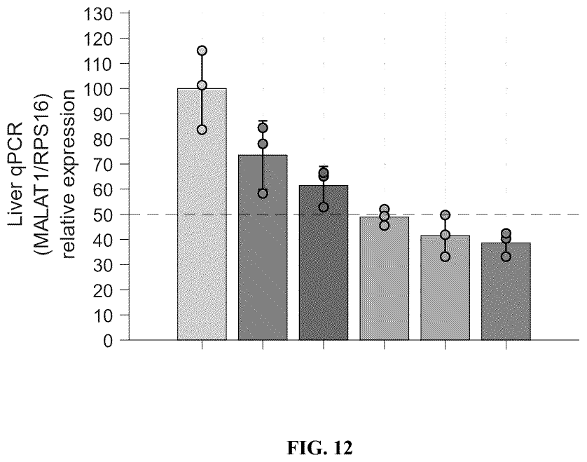

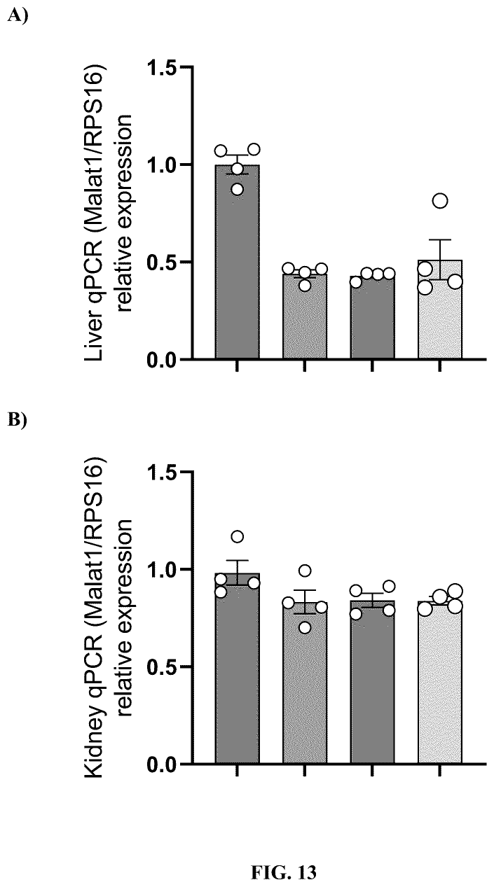

The relative mRNA levels in liver and kidney after treatment are shown in FIGS. 2 and 4 (normalized to the PBS control at a value of 1; column 1). The naked ASO (column 2) did not produce any significant change in mRNA levels, whereas Compound 1 yielded a significant knockdown in mRNA levels in liver (to less than 50%; column 3 of FIG. 2) but not in kidney (column 3 of FIG. 4). PK data also showed significant exposure of compounds in liver (FIG. 3) with minimum accumulation in kidney (FIG. 5). Gene expression data in liver also indicated a significant reduction in mRNA levels (around 40-70% reduction) in groups treated with Compounds 1, 2c, 3 and 5-11 as compared to the control groups (FIGS. 10 and 12). The reduction seen with the test compounds was better than the Control cluster in each case. Similar results were observed for mRNA knock-down in liver using Compounds 2c, 16a and 15a (FIG. 13A). Compounds 2c, 16a and 15a did not, however, result in an appreciable reduction in mRNA levels in kidney (FIG. 13B). The average bodyweight of mice administered Compounds 2c, 16a and 15a was not appreciably different from the control group (PBS only), and the average liver and spleen weights were, likewise, not appreciably different (data not shown). Plasma ALT levels were either unchanged or were slightly elevated in mice administered Compounds 2c, 16a and 15a, as compared with the control group (data not shown).

siRNA Knockdown of ANGPTL3 in Mice

Compound 2 and the Control siRNA cluster (Example 1), targeting Angptl3, were investigated in vivo in male C57BL6/J mice (10-11 weeks old with bodyweight 25-30 g) using a single subcutaneous dose of 0.5 mg/kg (n=5 per group). An additional control group dosed with phosphate-buffered saline (PBS) was also included in the study (n=5).

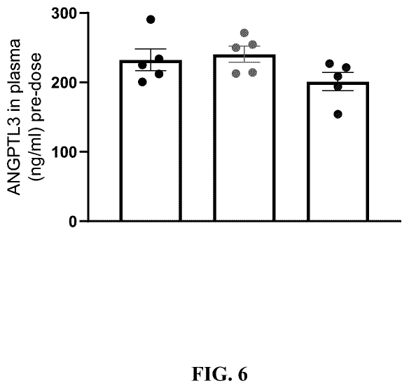

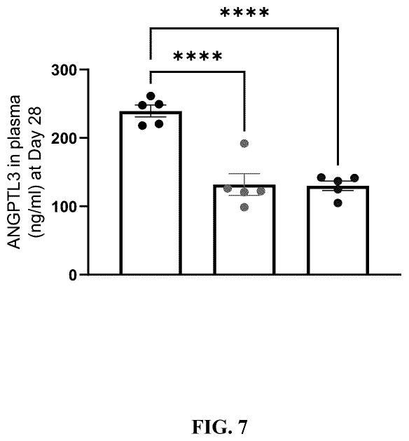

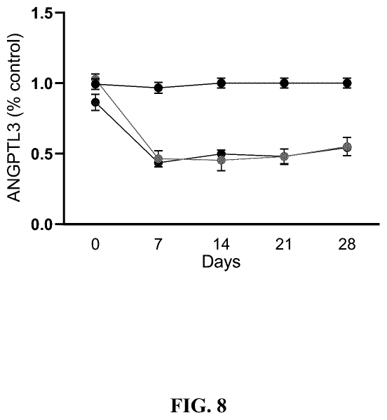

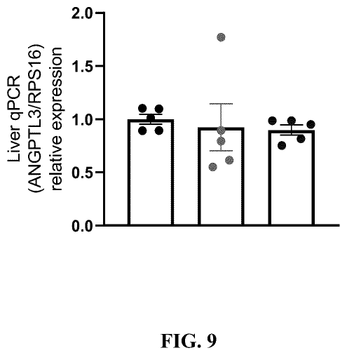

Blood samples were drawn pre-dose (FIG. 6) and 7, 14, and 21 days after dosing, as well as after animals were euthanized on day 28 after dosing (FIGS. 7 and 8). The ANGPTL3 protein concentration in circulation was measured by ELISA (Catalog Number MANL30; R&D Systems). Liver tissues were sampled on day 28 for measurement of Angptl3 mRNA levels by qPCR. Total RNA was extracted from cells or tissue samples using the RNeasy 96 QIAcube HT Kit (Qiagen). Complementary DNA (cDNA) was generated using a High-Capacity cDNA Reverse Transcription kit (Applied Biosystems). Reverse transcription qPCR analysis was performed with a QuantStudio 7 Flex using ANGPTL3 TaqMan assays (Mouse Mm00803820, Applied Biosystems) following the manufacturer's instructions. Hepatic Angptl3 mRNA levels were normalized over the mean of RPS16 mRNA levels and then normalized to PBS control (FIG. 9).

The primary readout in form of ANGPTL3 protein data indicated no statistical difference in baseline levels between groups, with baseline ANGPTL3 protein around 200 ng/mL corresponding to the typical level observed in historical data for this in vivo model. By Day 28, we observed a statistically significant reduction in ANGPTL3 protein concentrations with both the Control cluster and Compound 2 groups relative to PBS. The baseline and control corrected data indicate around a 50% reduction in ANGPTL3 protein after one week with this reduction sustained up to Day 21 with both Control cluster and Compound 2 groups. A 40% reduction in ANGPTL3 protein after Day 28 would indicate reduced potency of the compounds and potentially the slow return to baseline concentrations. For the secondary readout, expression level of Angptl3 mRNA in liver homogenate, data indicated no significant knockdown of the mRNA levels for the treated groups compared to the control group. While this observation was unexpected, this may indicate a recovery in hepatic mRNA production with a premature effect on protein levels (given the residual reduction of circulating ANGPTL3 protein at Day 28.)

siRNA Knockdown of PPIB in Mice

Compounds 2b & 12 and the control siRNA cluster (Example 1), targeting PPIB, were investigated in vivo in male C57BL6/J mice (10-11 weeks old with bodyweight 25-30 g) using a single subcutaneous dose of 3 mg/kg (n=4 per group). An additional control group dosed with phosphate-buffered saline (PBS) was also included in the study (n=4).

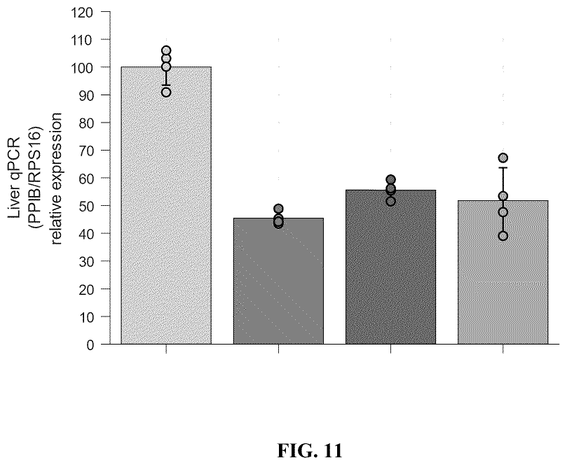

Blood samples were drawn at point of termination where animals were euthanized on Day 7 after dosing. Liver tissues were sampled on Day 7 for measurement of PPIB mRNA levels by qPCR. Total RNA was extracted from cells or tissue samples using the RNeasy 96 QIAcube HT Kit (Qiagen). Complementary DNA (cDNA) was generated using a High-Capacity cDNA Reverse Transcription kit (Applied Biosystems). Reverse transcription qPCR analysis was performed with a QuantStudio 7 Flex using PPIB TaqMan assays (Mouse Mm00478295, Applied Biosystems) following the manufacturer's instructions. Hepatic PPIB mRNA levels were normalized over the mean of RPS16 mRNA levels and then normalized to PBS control (FIG. 11).

The primary readout in the form of PPIB gene expression data in liver homogenate at Day 7 indicated a statistically significant reduction in PPIB mRNA levels (˜50%) in the treated groups compared to the control group (FIG. 11). The reduction seen with Compounds 2b & 12 is on a par with that of the Control cluster.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A compound having the structure of Formula (I): 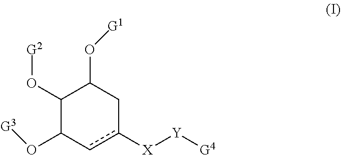
or a pharmaceutically acceptable salt thereof, wherein:
X is selected from a covalent bond, —CH₂—, and —C(O)—;
Y is —O— or —NR—,
wherein R is selected from —H and —(C₁-C₆)alkyl; [symbol]
represents a carbon-carbon single bond or double bond, with the proviso that when X is a covalent bond [symbol] represents a carbon-carbon single bond; and
G¹, G², G³, and G⁴ each independently represents a -[spacer]-[ligand] moiety or a -[tether]-[linker]-[cargo] moiety, wherein each ligand is independently a monosaccharide and each cargo is independently a nucleic acid, with the proviso that at least one of G¹, G², G³, and G⁴ represents a -[spacer]-[ligand] moiety and at least one of G¹, G², G³, and G⁴ represents a -[tether]-[linker]-[cargo] moiety.

2. The compound, or the pharmaceutically acceptable salt thereof, of claim 1, wherein X is —C(O)—.

3. The compound, or the pharmaceutically acceptable salt thereof, of claim 1, wherein Y is —NR—.

4. (canceled)

5. The compound, or the pharmaceutically acceptable salt thereof, of claim 1, wherein each ligand is N-acetyl galactosamine (GalNAc).

6. The compound, or the pharmaceutically acceptable salt thereof, of claim 1, wherein each cargo is independently selected from an antisense oligonucleotide, an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide, and a G-quadruplex.

7. The compound, or the pharmaceutically acceptable salt thereof, of claim 1, wherein each spacer independently comprises a chain of 2 to 20 atoms selected from C, N, O, S, and P.

8. The compound, or the pharmaceutically acceptable salt thereof, of claim 1, wherein linker and tether taken together in each case independently represents a moiety comprising a linear chain of 8 to 30 atoms.

9. The compound, or the pharmaceutically acceptable salt thereof, of claim 1, wherein the compound has the structure of Formula (Ia) or Formula (Ib): 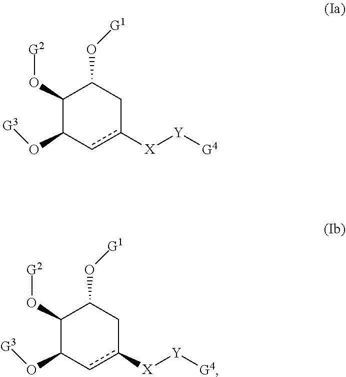
wherein X, Y, G¹, G², G³, and G⁴ are as defined in claim 1, and wherein [symbol] in Formula (Ib) represents a double bond.

10. The compound, or the pharmaceutically acceptable salt thereof, of claim 1, wherein the compound has the structure of Formula (Ia) or Formula (Id): 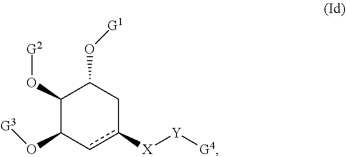
wherein X, Y, G¹, G², G³, and G⁴ are as defined in claim 1, and [symbol] represents a double bond.

11. A compound having the structure of Formula (II): 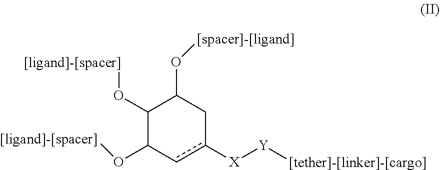
or a pharmaceutically acceptable salt thereof, wherein:
X is selected from a covalent bond, —CH₂—, and —C(O)—;
Y is —O— or —NR—,
wherein R is selected from —H and —(C₁-C₆)alkyl; [symbol]
represents a carbon-carbon single bond or double bond, with the proviso that when X is a covalent bond [symbol] represents a carbon-carbon single bond;
spacer in each case independently represents a moiety comprising a linear chain of 6 to 20 atoms which attaches the ligand to the rest of the molecule;
ligand in each case is independently a monosaccharide;
linker and tether taken together represent a moiety which attaches the cargo to the rest of the molecule; and
cargo is a nucleic acid.

12. The compound, or the pharmaceutically acceptable salt thereof, of claim 11, wherein X is —C(O)— and Y is —NR—.

13. The compound, or the pharmaceutically acceptable salt thereof, of claim 11, wherein [symbol] represents a single bond.

14. The compound, or the pharmaceutically acceptable salt thereof, of claim 11, wherein the compound has the structure of Formula (IIa) or Formula (IIb): 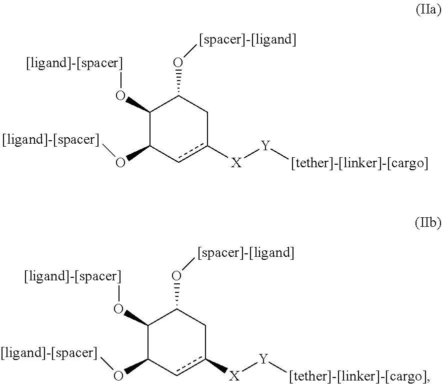
wherein X, Y, ligand, spacer, tether, linker, and cargo are as defined in claim 11, and wherein [symbol] in Formula (IIb) represents a carbon-carbon single bond.

15. The compound, or the pharmaceutically acceptable salt thereof, of claim 11, wherein the compound has the structure of Formula (IId): 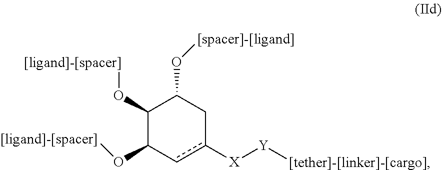
wherein X, Y, ligand, spacer, tether, linker, and cargo are as defined in claim 11, and [symbol] represents a double bond.

16. A compound having the structure of Formula (III): 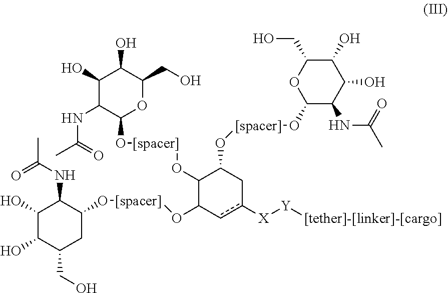
or a pharmaceutically acceptable salt thereof, wherein:
X is selected from a covalent bond, —CH₂—, and —C(O)—;
Y is —O— or —NR—,
wherein R is selected from —H and —(C₁-C₆)alkyl; [symbol]
represents a carbon-carbon single bond or double bond, with the proviso that when X is a covalent bond [symbol] represents a carbon-carbon single bond;
spacer in each case independently represents a moiety comprising a linear chain of 6 to 20 atoms;
linker and tether taken together represent a moiety which attaches the cargo to the rest of the molecule; and
cargo is a nucleic acid.

17. The compound, or the pharmaceutically acceptable salt thereof, of claim 16, wherein:
X is —C(O)—;
Y is —O— or —NR—,
wherein R is selected from —H and —(C₁₋₆)alkyl; [symbol]
represents a single bond;
spacer in each case independently represents a moiety comprising a linear chain of 6 to 20 atoms selected from C, N, O, S, and P;
linker and tether taken together represent a moiety comprising a linear chain of 8 to 30 atoms; and
cargo is a nucleic acid.

18. The compound, or the pharmaceutically acceptable salt thereof, of claim 16, wherein the compound has the structure of Formula (IIIa) or Formula (IIIb): 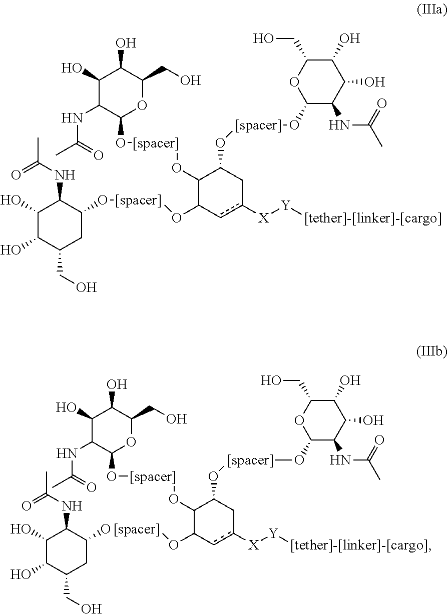
wherein X, Y, spacer, tether, linker, and cargo are as defined in claim 16, and wherein [symbol] in Formula (IIIb) represents a carbon-carbon single bond.

19. The compound, or the pharmaceutically acceptable salt thereof, of claim 16, wherein the compound has the structure of Formula (IIId): 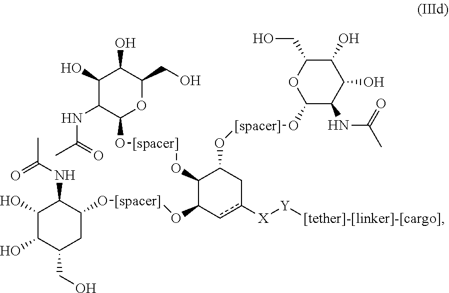
wherein X, Y, spacer, tether, linker, and cargo are as defined in claim 16, and [symbol] represents a double bond.

20.-28. (canceled)

29. A compound having the structure of Formula (IX): 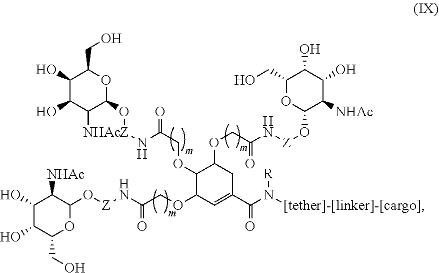
or a pharmaceutically acceptable salt thereof, wherein:
Z in each case independently represents a moiety comprising a linear chain of 1 to 16 atoms which attaches the GalNAc ligand to the rest of the molecule;
m in each case is independently selected from the integers from 1 to 6;
R is selected from —H and —(C₁-C₆)alkyl;
linker and tether taken together represent a moiety which attaches the cargo to the rest of the molecule; and
cargo is a nucleic acid.

30. A compound having the structure of Formula (XI): 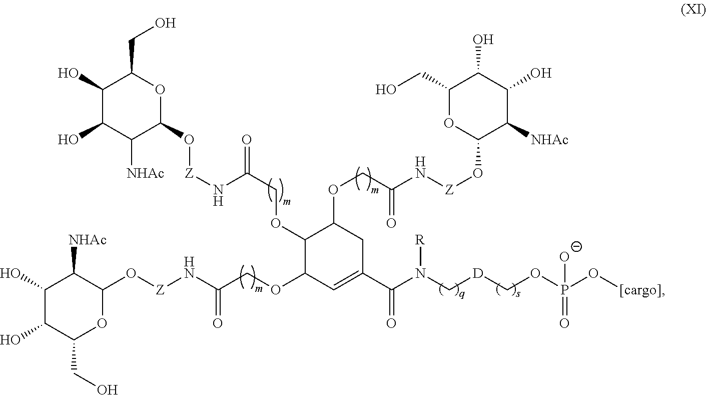
or a pharmaceutically acceptable salt thereof, wherein:
Z in each case is independently selected from *—(C₁-C₁₂)alkylene- and *—(C₁-C₆)alkylene-C(O)NR′—(C₁-C₄)alkylene— wherein R′ in each case is independently selected from —H and —(C₁-C₆)alkyl, and * in each case denotes the point of attachment to the GalNac oxygen atom;
m in each case is independently selected from the integers from 1 to 6;
R is selected from —H and —(C₁-C₆)alkyl;
q is an integer from 1 to 12;
D is selected from (i) —C(O)NH—, and (ii) —NHC(O)—(CH₂)ₜ—C(O)NH— wherein t is an integer from 1 to 6;
s is an integer from 0 to 12; and
cargo is a nucleic acid.

31. A compound selected from: 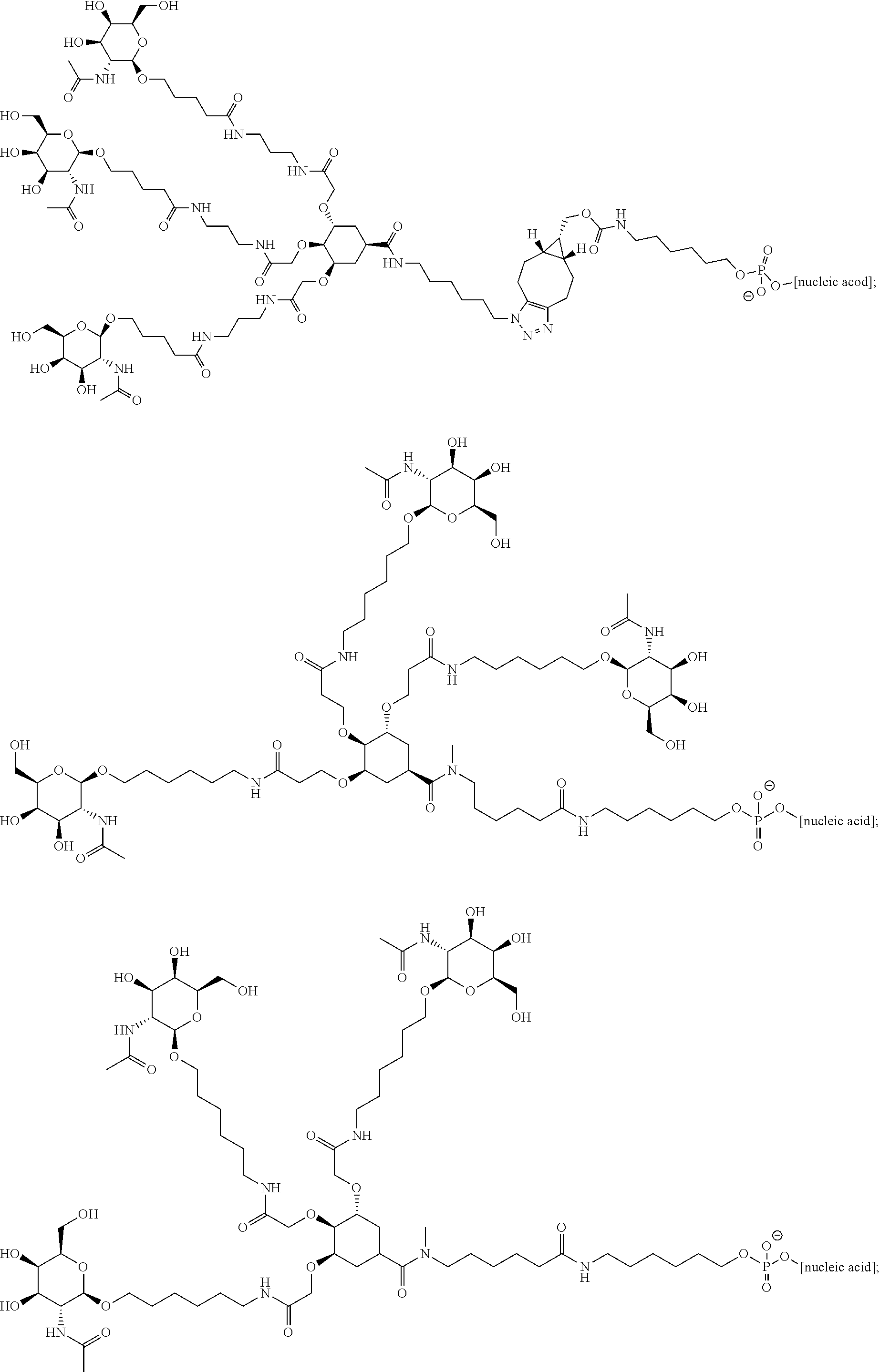 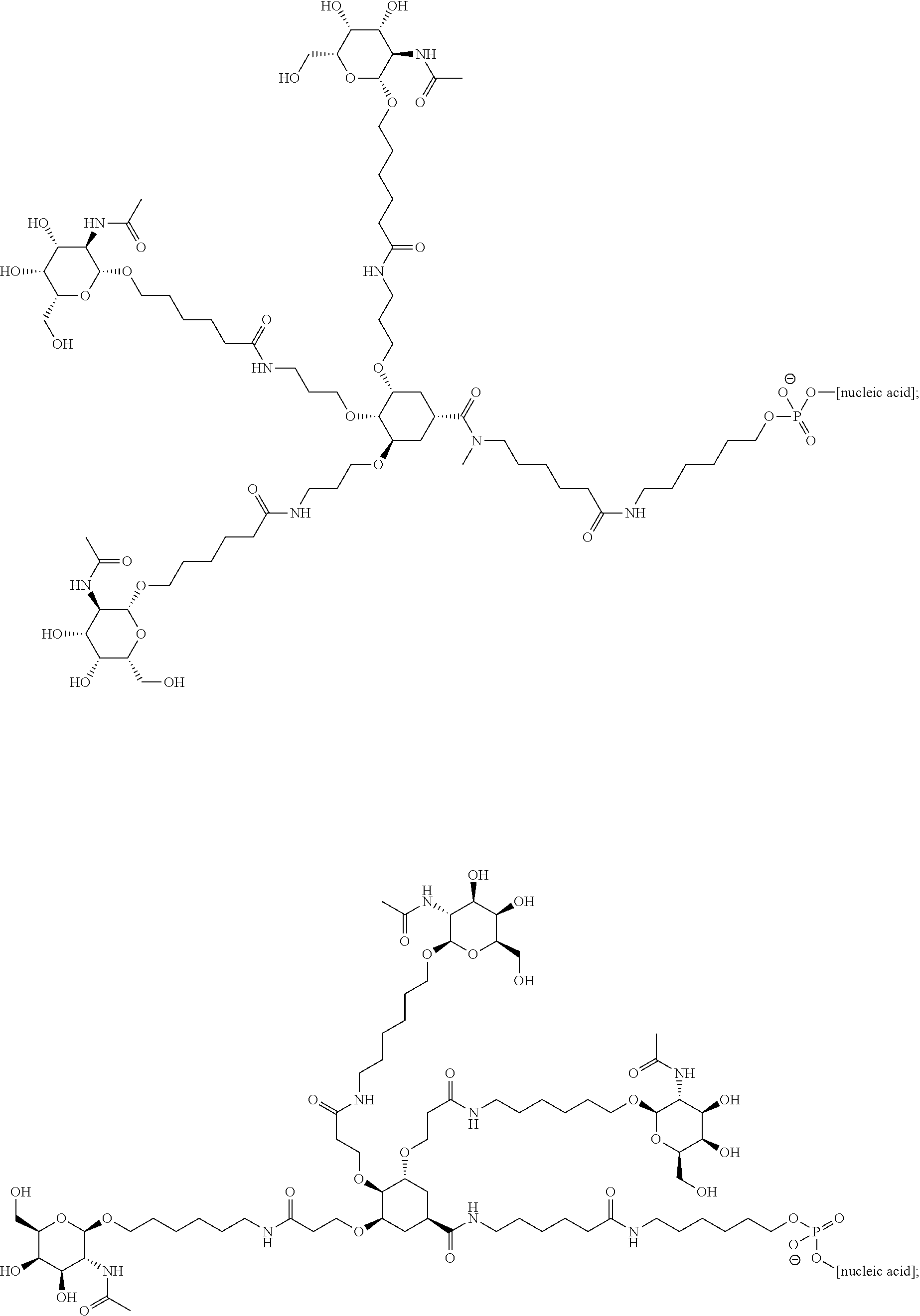 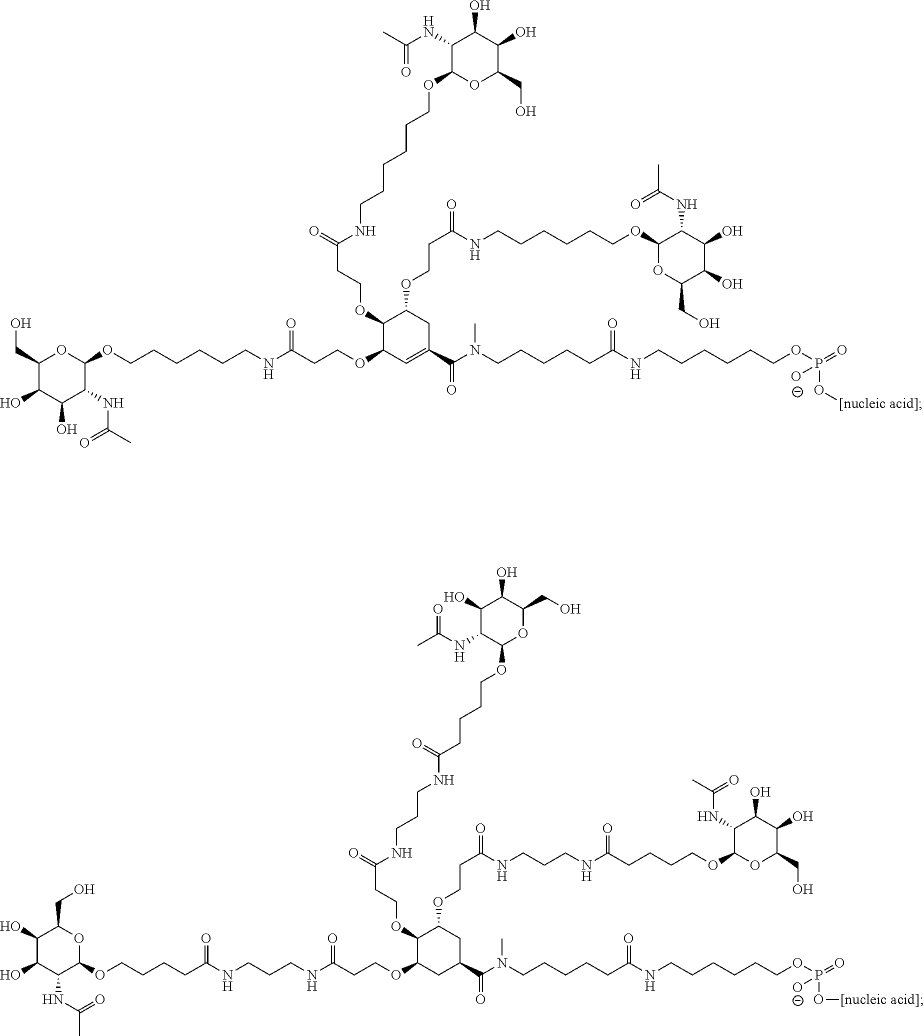 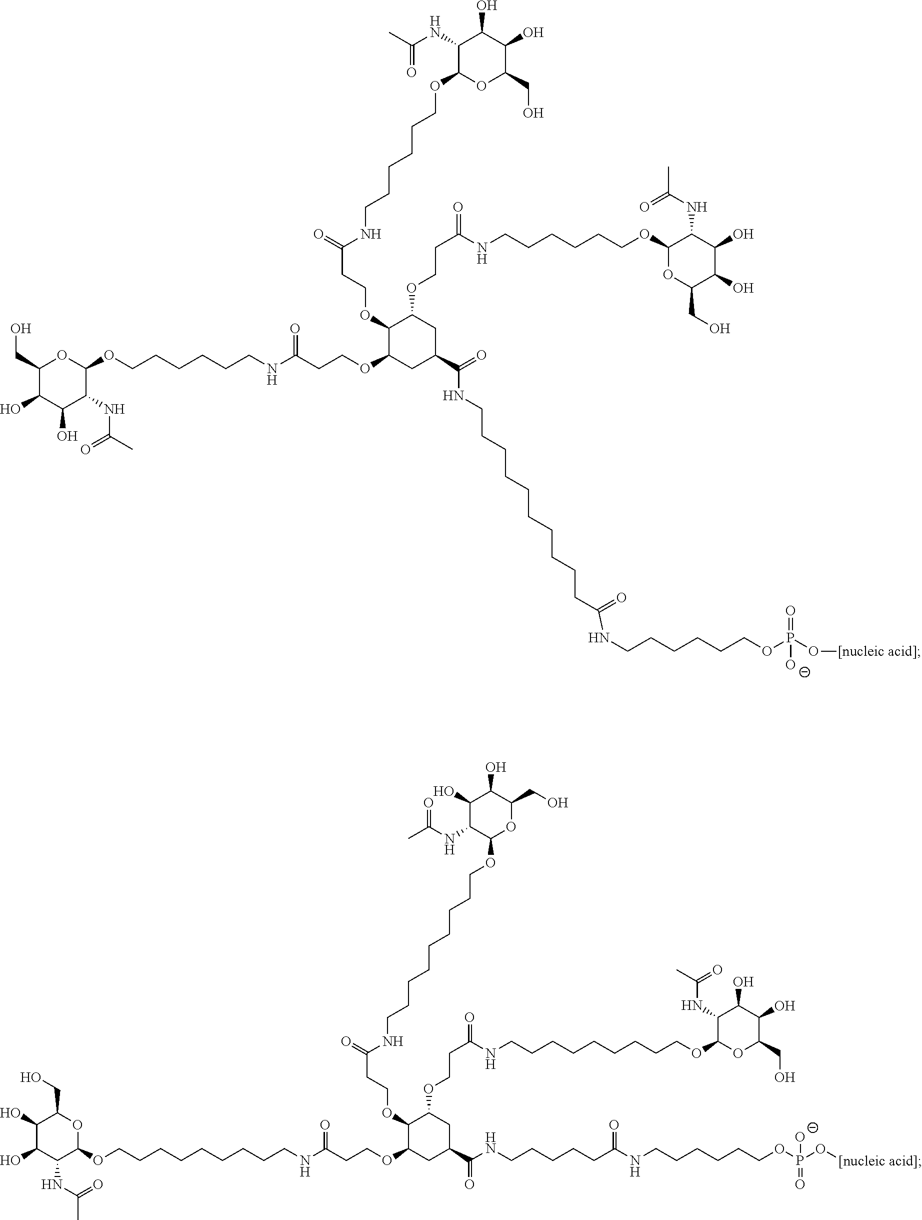 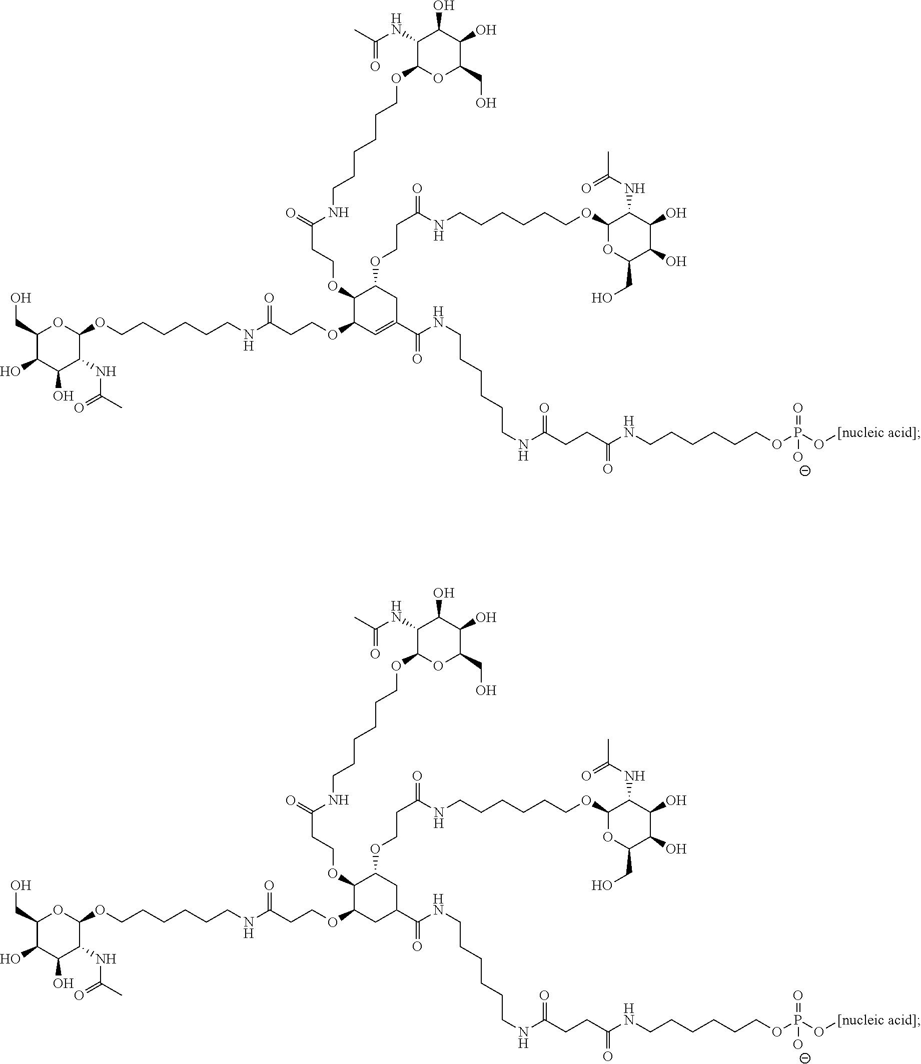 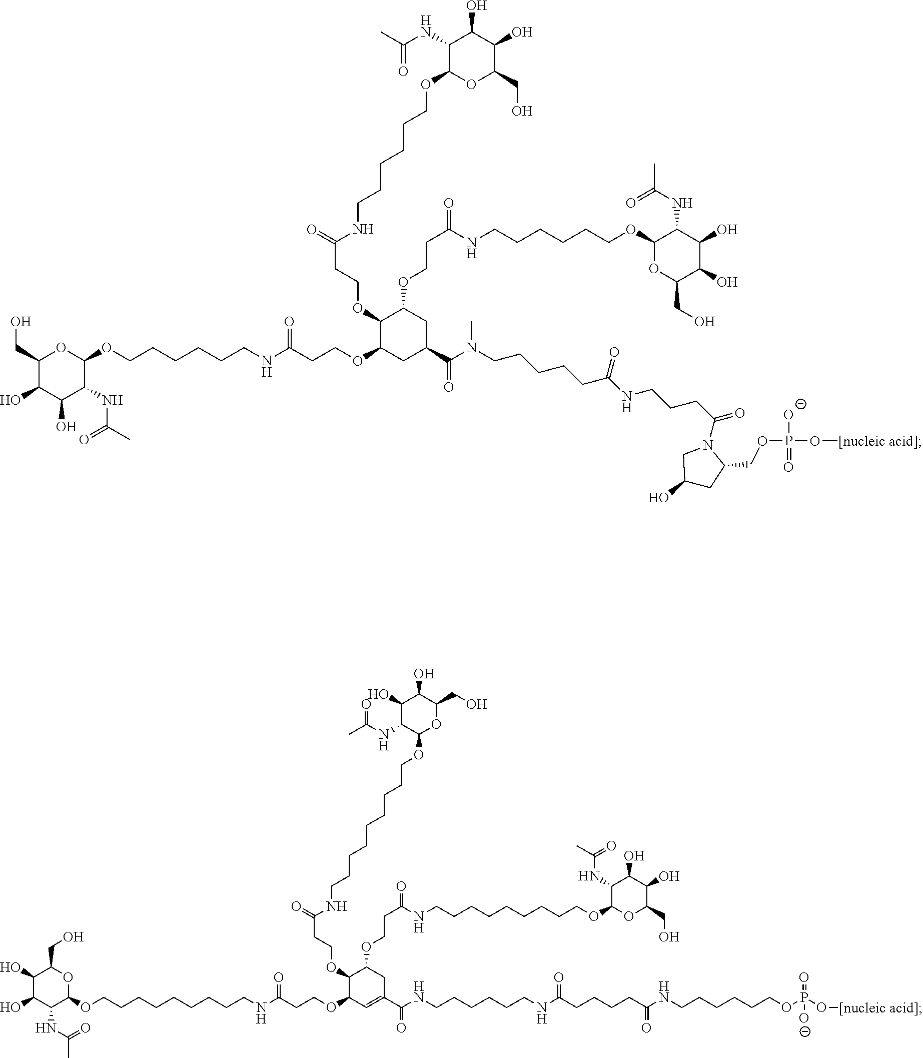 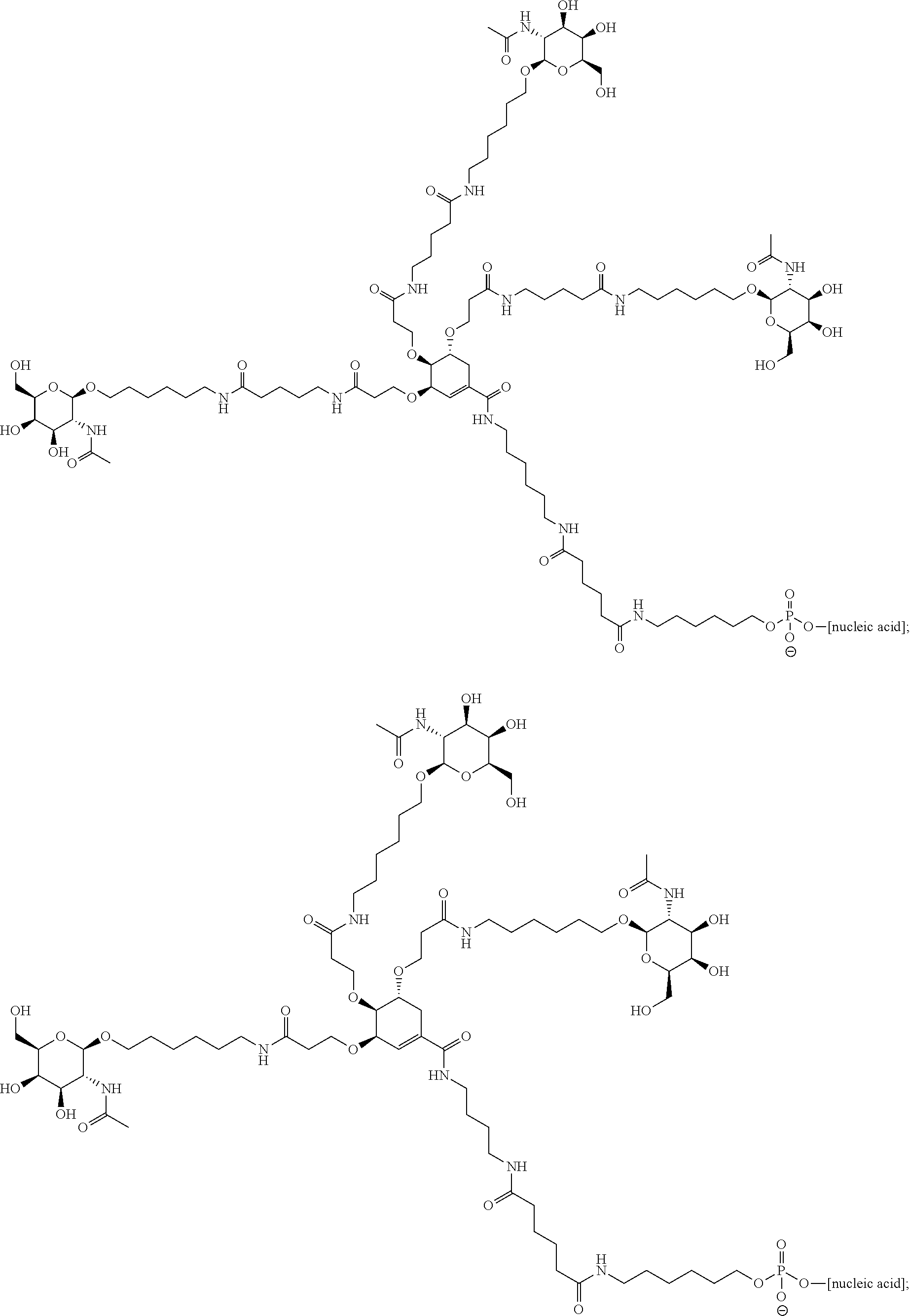 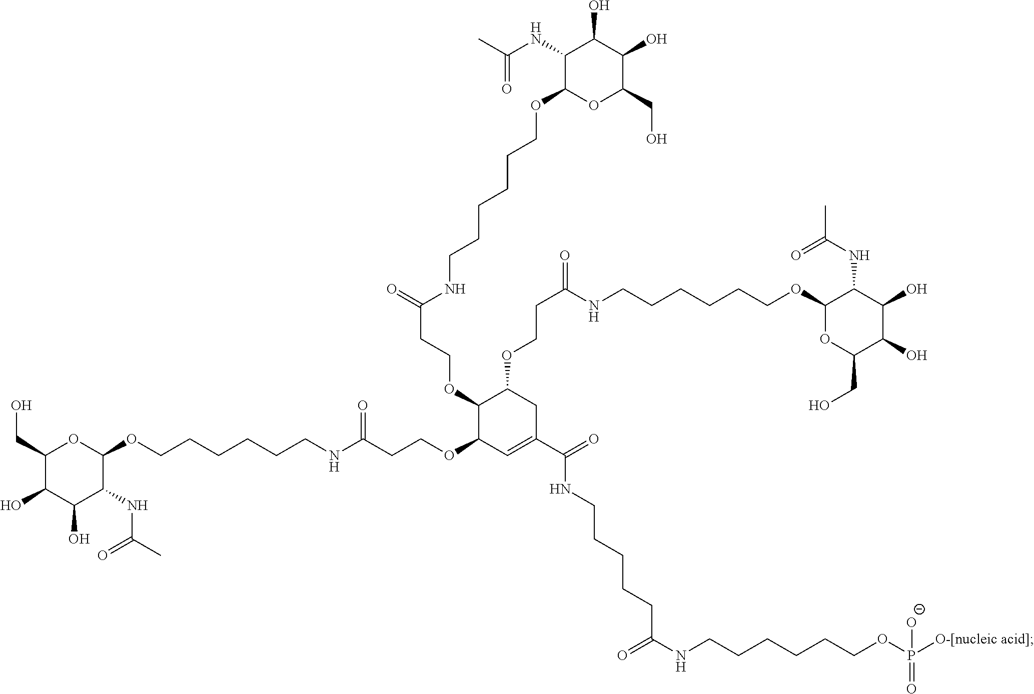 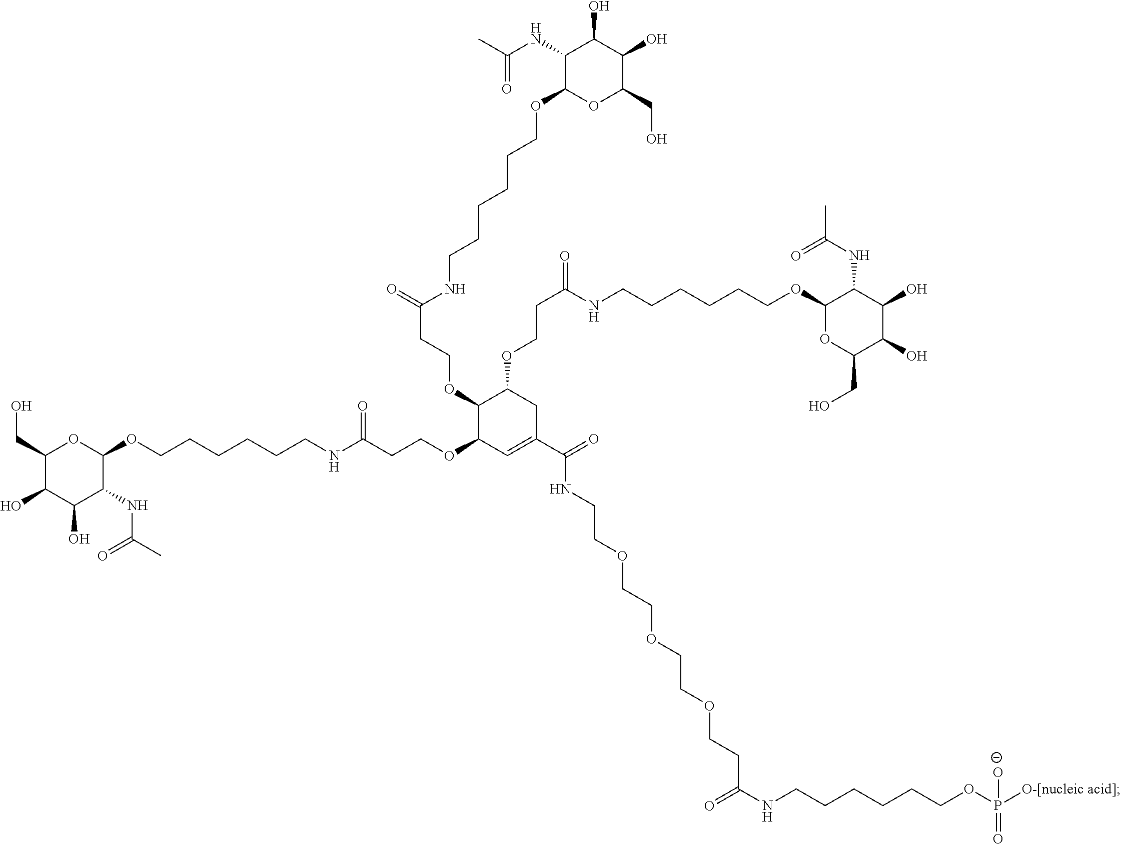 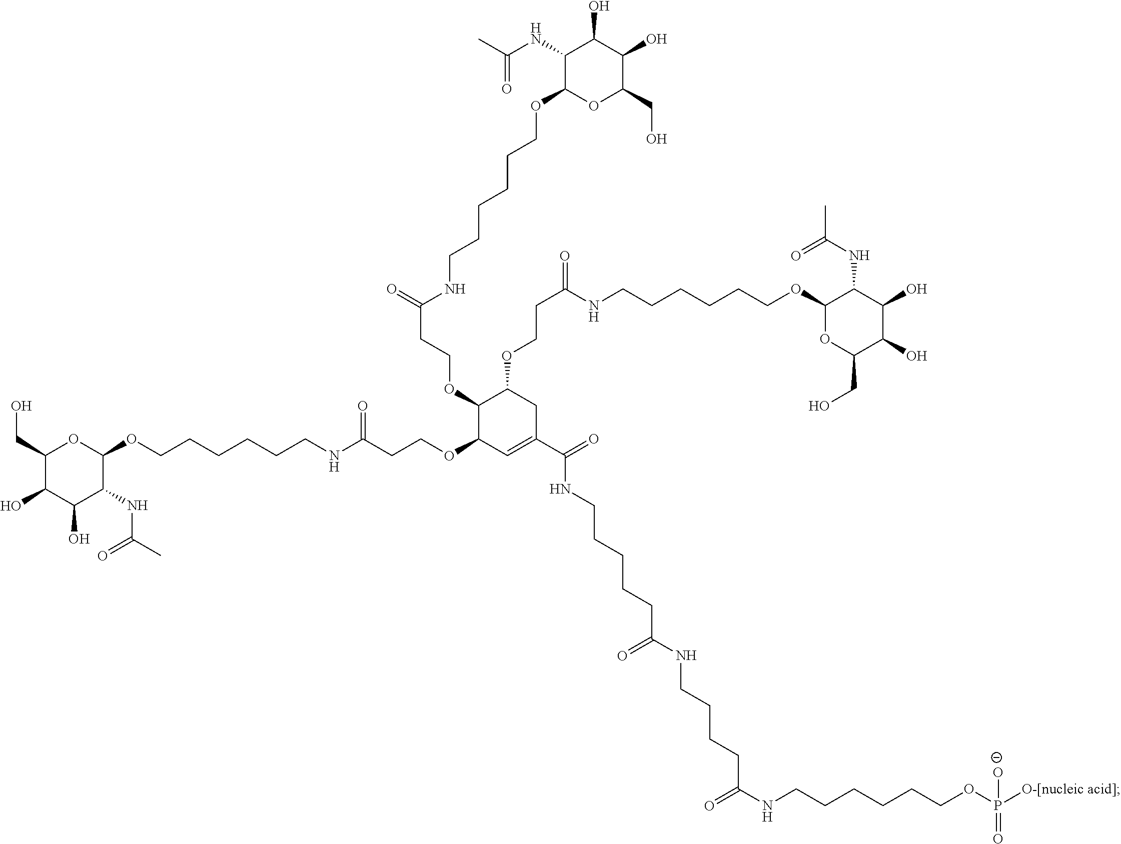 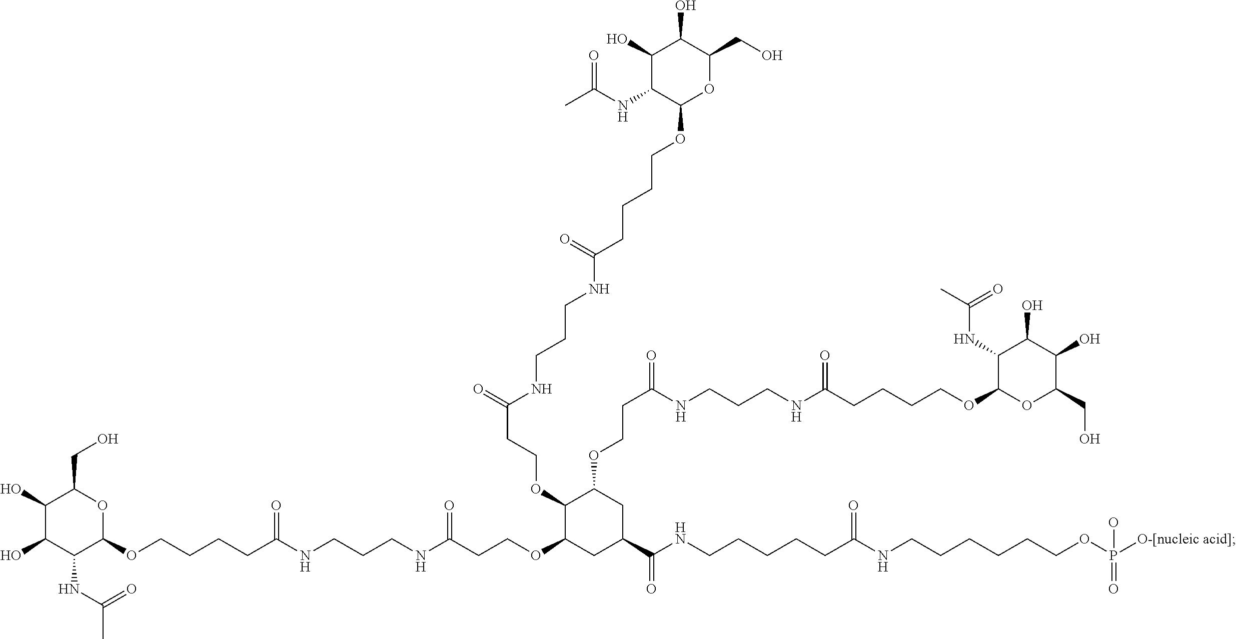 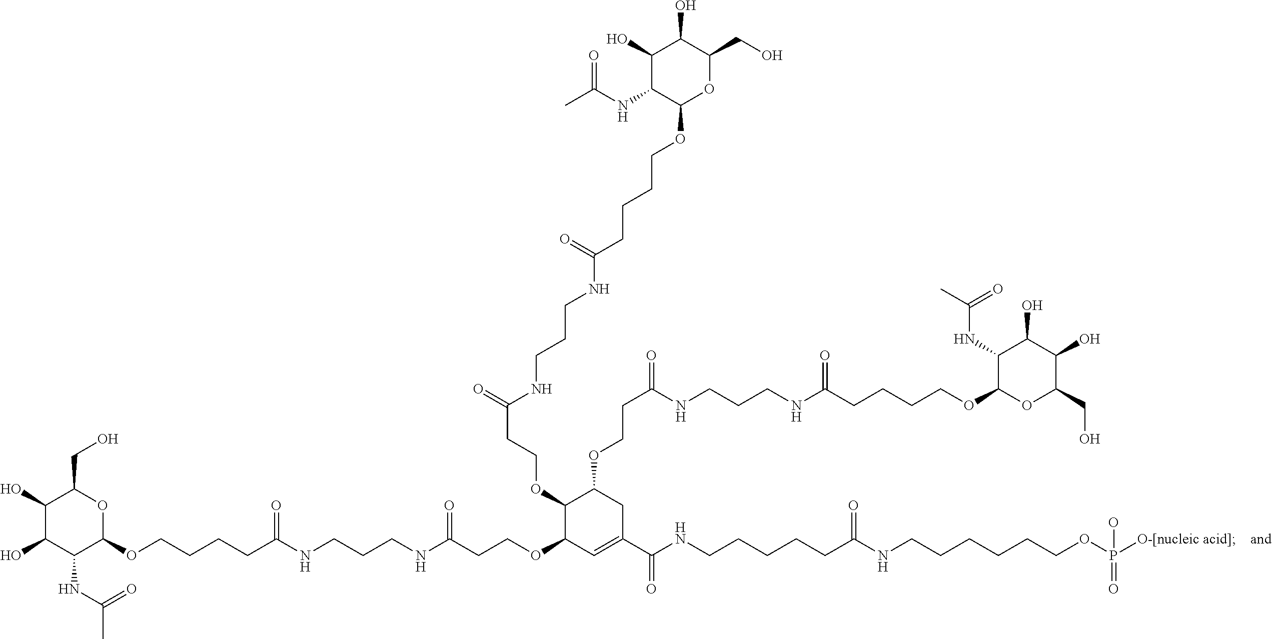 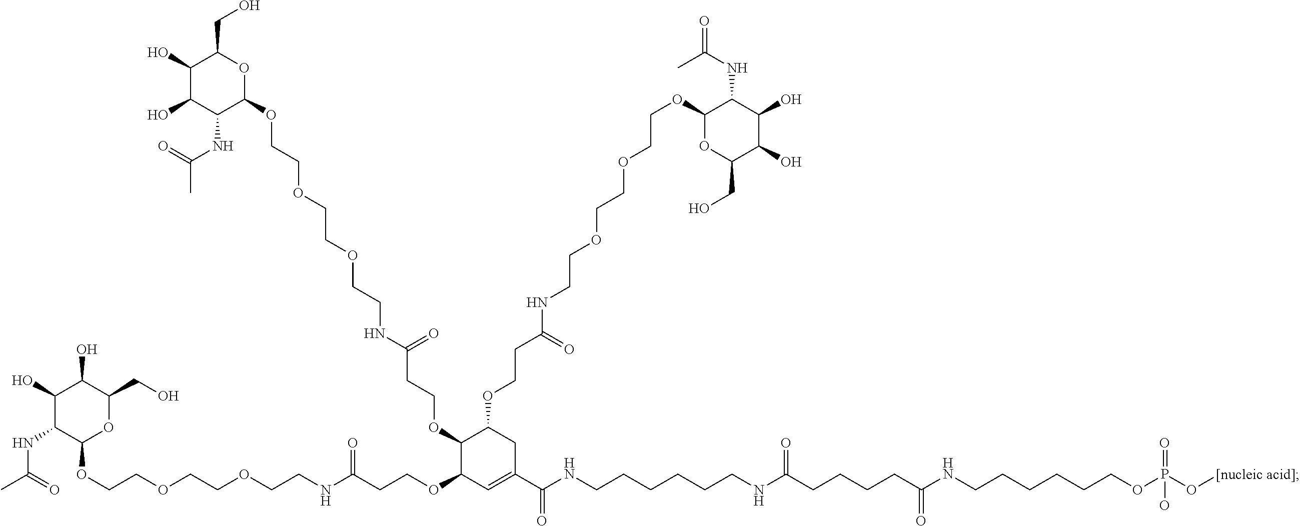
or a pharmaceutically acceptable salt thereof.

32. The compound, or the pharmaceutically acceptable salt thereof, of claim 31, wherein nucleic acid denotes an antisense oligonucleotide or siRNA.

33.-36. (canceled)

37. A pharmaceutical composition comprising a compound, or a pharmaceutically acceptable salt thereof, of claim 1, and at least one pharmaceutically acceptable excipient or carrier.

38. (canceled)

39. A method of treating a condition in a subject in need thereof, the method comprising administering an effective amount of a compound, or a pharmaceutically acceptable salt thereof, of claim 1, or a pharmaceutical composition of claim 37, to the subject, wherein the condition is selected from liver disease, genetic disease, haemophilia and bleeding disorders, liver fibrosis, non-alcoholic steatohepatitis, non-alcoholic fatty liver disease, viral hepatitis, rare diseases, metabolic disease, cardiovascular disease, obesity, thalassemia, liver injury, hemochromatosis, alcoholic liver disease, alcohol dependence, anaemia, and anaemia of chronic disease.

40. The method of claim 39, wherein the condition is selected from non-alcoholic steatohepatitis, non-alcoholic fatty liver disease, a metabolic disease, and a cardiovascular disease.

41. The method of treating of claim 39, wherein the condition is non-alcoholic steatohepatitis.

42. The method of claim 39, wherein the condition is a metabolic disease selected from hypercholesterolemia, dyslipidaemia, and hypertriglyceridemia.

43. A compound having the structure of Formula (VI): 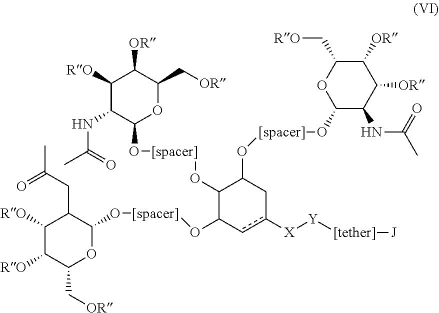
or a pharmaceutically acceptable salt thereof, wherein:
X is selected from a covalent bond, —CH₂—, and —C(O)—;
Y is —O— or —NR—,
wherein R is selected from —H and —(C₁-C₆)alkyl;
spacer in each case independently represents a moiety comprising a linear chain of 6 to 20 atoms selected from C, N, O, S, and P;
tether represents a moiety which attaches the “J” group to the “Y” group; [symbol]
represents a carbon-carbon single bond or double bond, with the proviso that when X is a covalent bond [symbol] represents a carbon-carbon single bond;
R″ is acyl, —C(O)aryl, or —H; and
J is —CO₂H, —OH, —C(O)O-(pentafluorophenyl), —N₃, or a phosphoramidite.

44.-47. (canceled)

48. A compound having the structure of Formula (XII): 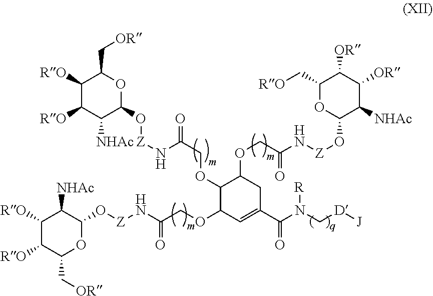
or a pharmaceutically acceptable salt thereof, wherein:
Z in each case is independently selected from *(C₁-C₁₂)alkylene- and *—(C₁-C₆)alkylene-C(O)NR′—(C₁-C₄)alkylene-; wherein R′ in each case is independently selected from —H and —(C₁-C₆)alkyl, and * in each case denotes the point of attachment to the adjacent oxygen atom;
R is selected from —H and —(C₁-C₆)alkyl;
m in each case is independently selected from the integers from 1 to 6;
q is an integer from 1 to 12;
D′ is selected from (i) a bond, and (ii) —NHC(O)—(CH₂)ₜ— wherein t is an integer from 1 to 6;
R″ is acyl, —C(O)aryl, or —H; and
J is —CO₂H, —OH, —C(O)O-(pentafluorophenyl), —N₃, or a phosphoramidite.

49.-50. (canceled)
